(19) United States
(12) Patent Application Publication
(10) Pub. No.: US 2024/0360427 A1
(43) Pub. Date: Oct. 31, 2024

(54) TARGETING ONCOGENIC MUTATIONS WITH DUAL-CLEAVING ENDONUCLEASE

(71) Applicant: SPECIFIC BIOLOGICS INC., Toronto (CA)

(72) Inventor: Brent E. STEAD, Toronto (CA)

(21) Appl. No.: 18/473,042

(22) Filed: Sep. 22, 2023

Related U.S. Application Data
(63) Continuation of application No. PCT/IB2022/000155, filed on Mar. 25, 2022.
(60) Provisional application No. 63/166,763, filed on Mar. 26, 2021.

(51) Int. Cl. C12N 9/22; A61K 48/00; A61P 35/00; C12N 15/10; C12N 15/11

(52) U.S. Cl. CPC C12N 9/22; A61K 48/0066; A61P 35/00; C12N 15/102; C12N 15/111; C12N 2310/20

(57) ABSTRACT

Provided herein are compositions and methods of using chimeric nucleases comprising an I-TevI nuclease domain and a Cas domain for the targeting of oncogenes.

### CROSS-REFERENCE TO RELATED APPLICATIONS

The present application is a Continuation Application based on International Application No. PCT/IB2022/000155, filed on Mar. 25, 2022, which claims priority to U.S. Provisional Patent Application No. 63/166,763, filed on Mar. 26, 2021, the disclosures of which are incorporated by reference herein in their entirety, including any drawings.

### INCORPORATION BY REFERENCE OF SEQUENCE LISTING

This application contains a Sequence Listing, which is incorporated by reference in its entirety. The accompanying Sequence Listing text file, name, “2023-12-28 Sequence_Listing_ST26 062709-503C01US.xml”, was created on Dec. 28, 2023 and is 1,636 KB.

### BACKGROUND OF THE INVENTION

Cancer is among the leading causes of death worldwide. In 2018, there were 18.1 million new cases and 9.5 million cancer-related deaths worldwide. By 2040, the number of new cancer cases per year is expected to rise to 29.5 million and the number of cancer-related deaths to 16.4 million. A proto-oncogene is a gene that has the potential to cause cancer. Once mutated, a proto-oncogene becomes an oncogene. In tumor cells, proto-oncogenes are often mutated, or expressed at high levels and can contribute to uncontrolled cell growth which is a hallmark of cancer. Many current therapeutics target the mutated protein expressed from an oncogene but there are no therapeutics that target the oncogene itself. It is therefore important to develop new technologies to disrupt an oncogene.

### SUMMARY OF THE INVENTION

Described herein are chimeric nucleases comprising an I-TevI domain (1), a Cas domain, and a guide RNA targeting the chimeric nuclease to an oncogenic mutation. Such chimeric nucleases advantageously allow for precise targeting and editing of the genome of a cell to restore a non-oncogenic function of an oncogene. Compared to use of Cas enzymes alone the inclusion of the I-TevI domain allows for more precise editing and replacement of oncogenic sequences in cancer cells.

In an aspect, the present disclosure provides a composition comprising: a chimeric nuclease, wherein the chimeric nuclease comprises an I-TEVI nuclease domain, an RNA-guided nuclease Cas domain, and a guide RNA, wherein the guide RNA comprises a nucleic acid sequence that targets an oncogenic mutation that is not a deletion in exon 19 of EGFR.

In some embodiments, the oncogenic mutation is a single nucleotide polymorphism. In some embodiments, a sequence comprising the oncogenic mutation is selected from a mutation set forth in any one of SEQ ID NOs: 1-683, or a combination thereof. In some embodiments, a sequence comprising the oncogenic mutation is at least 85%, 90%, 95%, 97%, 98%, or 99% identical to a mutation set forth in any one of SEQ ID NOs: 1-683, or a combination thereof. In some embodiments, the oncogenic mutation comprises a mutation corresponding an EGFR L858R mutation or an EGFR V769_D770insASV mutation. In some embodiments, the oncogenic mutation comprises a mutation corresponding to an EGFR L858R mutation. In some embodiments, the guide RNA hybridizes to a target nucleotide sequence set forth in SEQ ID NO: 45, 130, or 141, or comprises a nucleotide sequence as set forth in SEQ ID NO: 1045, I130, 1141, or 1686. In some embodiments, the guide RNA comprises a nucleic acid sequence that hybridizes to a target nucleic acid sequence at least 85%, 90%, 95%, 97%, 98%, or 99% identical to any one of SEQ ID NO: 45, 130, 141, or comprises a nucleotide sequence at least 85%, 90%, 95%, 97%, 98%, or 99% identical to any one of SEQ ID NO: 1045, I130, I141, or 1686. In some embodiments, the oncogenic mutation comprises a mutation corresponding to an EGFR V769_D770insASV mutation. In some embodiments, the guide RNA hybridizes to a target nucleotide sequence set forth in SEQ ID NO: 683, or comprises a nucleotide sequence as set forth in SEQ ID NO: 1683 or 1684. In some embodiments, the guide RNA comprises a nucleic acid sequence that hybridizes to a target nucleic acid sequence at least 85%, 90%, 95%, 97%, 98%, or 99% identical to any one of SEQ ID NO: 683, or comprises a nucleotide sequence at least 85%, 90%, 95%, 97%, 98%, or 99% identical to any one of SEQ ID NO: 1683 or 1684. In some embodiments, the oncogenic mutation is an oncogenic mutation to a gene selected from any of Muc4, PIK3CA, KRAS, or a combination there. In some embodiments, the oncogenic mutation comprises a Muc4 mutation. In some embodiments, the Muc4 mutation is an in-frame deletion of exon 2 or an in-frame deletion of exon 3. In some embodiments, the Muc4 mutation comprises a mutation corresponding to any one of positions P1542, P1680, T1711, V1721, P1826, A1830, S3560, A1833, D2253, V2281, P3088, T3119, T3183, V3817, A3902 of human Muc4 protein, or a combination thereof. In some embodiments, the Muc4 mutation is selected from a mutation corresponding to any one of P1542L, P1680S, T17111, V1721A, P1826H, A1830T, S3560S, A1833V, D2253H, V2281AM, P3088L, T3119T, T3183M, V3817A, A3902V of human Muc4 protein, or a combination thereof. In some embodiments, the guide RNA hybridizes to a target nucleotide sequence set forth in SEQ ID NO: 676, 677, 678, 679 or 682, or comprises a nucleotide sequence as set forth in SEQ ID NO: 1676, I677, I678, I679, I682, or 1685. In some embodiments, the guide RNA comprises a nucleic acid sequence that hybridizes to a target nucleic acid sequence at least 85%, 90%, 95%, 97%, 98%, or 99% identical to any one of SEQ ID NO: 676, 677, 678, 679 or 682, or comprises a nucleotide sequence at least 85%, 90%, 95%, 97%, 98%, or 99% identical to any one of SEQ ID NO: 1676, I677, I678, I679, 1682, or 1685. In some embodiments, the oncogenic mutation comprises a PIK3CA mutation. In some embodiments, the PIK3CA mutation comprises a mutation corresponding to any one of positions H1047, E542, E545, N345, C1636, G1624, G1633, A3140, C3075, A1634, A1173 of human PIK3A protein, or a combination thereof. In some embodiments, the PIK3CA mutation is selected from a mutation corresponding to any one of H1047R, H1047L, E542K, E545K, N345K, C1636A, G1624A, G1633A, A3140T, A3140G, C3075T, A1634C, A1173G of human PIK3A protein, or a combination thereof. In some embodiments, the guide RNA hybridizes to a target nucleotide sequence set forth in SEQ ID NO: 5, 6, 7, 8, 33, 202, 204, 209 or 210, or comprises a nucleotide sequence as set forth in SEQ ID NO: 1005, 1006, 1007, 1008, 1033, 1202, 1204, 1209, or 1210. In some embodiments, the guide RNA comprises a nucleic acid sequence that hybridizes to a target nucleic acid sequence at least 85%, 90%, 95%, 97%, 98%, or 99% identical to any one of SEQ ID NO: 5, 6, 7, 8, 33, 202, 204, 209 or 210, or comprises a nucleotide sequence at least 85%, 90%, 95%, 97%, 98%, or 99% identical to any one of SEQ ID NO: 1005, 1006, 1007, 1008, 1033, 1202, 1204, 1209, or 1210. In some embodiments, the oncogenic mutation comprises a KRAS mutation. In some embodiments, the KRAS mutation comprises a mutation selected from a mutation corresponding to any one of positions A59, D119, D33, G21, G12, G13, Q61, A146, K117 of human KRAS protein, or a combination thereof. In some embodiments, the KRAS mutation is selected from a mutation corresponding to any one of A59T, A59E, A59T, D119N, D33E, G21C, G12C, G12D, G12V, G12R, G12A, G12S, G13D, G13C, G13V, G13R, Q61R, Q61V, Q61L, Q61K, Q61H, Q61A, Q61P, Q61E, A146T, A146V, K117N, K117R of human KRAS protein, or a combination thereof. In some embodiments, the guide RNA hybridizes to a target nucleotide sequence set forth in SEQ ID NO: 37, 42, 51, 52, 62, 63, or 77, or comprises a nucleotide sequence as set forth in SEQ ID NO: 1037, 1042, 1051, 1052, 1062, 1063, or 1077. In some embodiments, the guide RNA comprises a nucleic acid sequence that hybridizes to a target nucleic acid sequence at least 85%, 90%, 95%, 97%, 98%, or 99% identical to any one of SEQ ID NO: 37, 42, 51, 52, 62, 63, or 77, or comprises a nucleotide sequence at least 85%, 90%, 95%, 97%, 98%, or 99% identical to any one of SEQ ID NO: 1037, 1042, 1051, 1052, 1062, 1063, or 1077. In some embodiments, the guide RNA comprises one or more of: a non-natural internucleoside linkage, a nucleic acid mimetic, a modified sugar moiety, or a modified nucleobase. In some embodiments, the non-natural internucleoside linkage comprises one or more of: a phosphorothioate, a phosphoramidate, a non-phosphodiester, a heteroatom, a chiral phosphorothioate, a phosphorodithioate, a phosphotriester, an aminoalkylphosphotriester, a 3′-alkylene phosphonates, a 5′-alkylene phosphonate, a chiral phosphonate, a phosphinate, a 3′-amino phosphoramidate, an aminoalkylphosphoramidate, a phosphorodiamidate, a thionophosphoramidate, a thionoalkylphosphonate, a thionoalkylphosphotriester, a selenophosphate, or a boranophosphate. In some embodiments, the nucleic acid mimetic comprises one or more of a peptide nucleic acid (PNA), morpholino nucleic acid, cyclohexenyl nucleic acid (CeNAs), or a locked nucleic acid (LNA). In some embodiments, the modified sugar moiety comprises one or more of 2′-O-(2-methoxyethyl), 2′-dimethylaminooxyethoxy, 2′-dimethylaminoethoxyethoxy, 2′-O-methyl, or 2′-fluoro. In some embodiments, the modified nucleobase comprises one or more of: a 5-methylcytosine; a 5-hydroxymethyl cytosine; a xanthine; a hypoxanthine; a 2-aminoadenine; a 6-methyl derivative of adenine; a 6-methyl derivative of guanine; a 2-propyl derivative of adenine; a 2-propyl derivative of guanine; a 2-thiouracil; a 2-thiothymine; a 2-thiocytosine; a 5-halouracil; a 5-halocytosine; a 5-propynyl uracil; a 5-propynyl cytosine; a 6-azo uracil; a 6-azo cytosine; a 6-azo thymine; a pseudouracil; a 4-thiouracil; an 8-halo; an 8-amino; an 8-thiol; an 8-thioalkyl; an 8-hydroxyl; a 5-halo; a 5-bromo; a 5-trifluoromethyl; a 5-substituted uracil; a 5-substituted cytosine; a 7-methylguanine; a 7-methyladenine; a 2-Fadenine; a 2-amino-adenine; an 8-azaguanine; an 8-azaadenine; a 7-deazaguanine; a 7-deazaadenine; a 3-deazaguanine; a 3-deazaadenine; a tricyclic pyrimidine; a phenoxazine cytidine; a phenothiazine cytidine; a substituted phenoxazine cytidine; a carbazole cytidine; a pyridoindole cytidine; a 7-deaza-adenine; a 7-deazaguanosine; a 2-aminopyridine; a 2-pyridone; a 5-substituted pyrimidine; a 6-azapyrimidine; an N-2, N-6 or 0-6 substituted purine; a 2-aminopropyladenine; a 5-propynyluracil; or a 5-propynylcytosine. In some embodiments, the composition further comprises a linker that is operably linked to the I-TEVI nuclease domain and the RNA-guided nuclease Cas domain. In some embodiments, the linker comprises an amino acid sequence as set forth in SEQ ID NO: 701, 702, 703, or 704. In some embodiments, the linker comprises a mutation corresponding to any one of positions T95, 5101, A119, K120, K135, P126, D127, N140, T147, Q158, A161, V117, 5165, or a combination thereof. In some embodiments, the linker comprises a mutation selected from a mutation corresponding to any one of T95S, S101Y, A119D, K120N, K135N, K135R, P126S, D127K, N140S, T1471, Q158R, A161V, V117F, S165G, or a combination thereof. In some embodiments, the linker comprises an amino acid sequence that is at least 85%, 90%, 95%, 97%, 98%, or 99% identical to SEQ ID NO: 701, 702, 703, or 704. In some embodiments, the linker comprises a mutation corresponding to any one of positions T95, S101, A119, K120, K135, P126, D127, N140, T147, Q158, A161, V117, S165, or a combination thereof. In some embodiments, the linker comprises a mutation selected from a mutation corresponding to any one of T95S, S101Y, A119D, K120N, K135N, K135R, P126S, D127K, N140S, T147I, Q158R, A161V, V117F, S165G, or a combination thereof. In some embodiments, the RNA-guided nuclease Cas domain is a RNA-guided nuclease Cas9 domain. In some embodiments, the RNA-guided nuclease Cas9 domain is any one of an RNA-guided nuclease Staphylococcus aureus Cas9 domain, an RNA-guided nuclease Streptococcus pyogenes Cas9 domain, an RNA-guided nuclease Neisseria meningitidis Cas9 domain, an RNA-guided nuclease Campylobacter jejuni Cas9 domain, an RNA-guided nuclease Streptococcus pasteurianus Cas9 domain, an RNA-guided nuclease Streptococcus pasteurianus Cas9 domain, an RNA-guided nuclease Clostridium cellulolyticum Cas9 domain, an RNA-guided nuclease Geobacillus thermodenitrificans T1 Cas9 domain, or combination thereof. In some embodiments, the RNA-guided nuclease Cas9 domain is an RNA-guided nuclease Staphylococcus aureus Cas9 domain. In some embodiments, the RNA-guided nuclease Staphylococcus aureus Cas9 domain comprises an amino acid sequence as set forth in SEQ ID NO: 710. In some embodiments, the RNA-guided nuclease Staphylococcus aureus Cas9 domain comprises a mutation corresponding to any one of positions D10, H557, N580, H840, D1135, R1335, T1337, T267, L325, V327, D333, A336, 1341, E345, D348, K352, S360, T368, N369, N371, S372, E373, K386, N393, H408, N410, 1414, A415, T438, Y467, N471, D485, M489, E506, R409, T510, N515, Y518, A539, F550, N551, S596, T602, A611, I617, T620, G654, N667, R685, K695, 1706, K722, A723, K724, M731, F732, K735, S739, P741, E742, E746, Q747, 1754, T755, H757, K760, H761, P778, E781, 1783, N784, D785, T786L, L787, Y788, K792, D794, T798, L799, V801, N803, L804, N805, G806, D813, K814, L818, 1819, S822, E824, L841, G847, D848, Y857, V875, 1876, N884, A888, L890, D894, D895, P897, V903, G920, F924, N929, E936, N937, V941, N942, S943, C945, E947, K951, L952, S956, N957, Q958, A959, N974, G975, V983, N984, N985, D986, I991, V993, M995, I996, T999, Y1000, R1001, E1002, L1004, E1005, N1006, M1007, D1009, K1010, R1011, P1012, P1013, I1015, 11016, A1020, S1021, Q1024, K1027, E1039, H1045, 10148, K1050 or a combination thereof. In some embodiments, the RNA-guided nuclease Staphylococcus aureus Cas9 domain comprises a mutation selected from a mutation corresponding to any one of D10A, D10E, H557A, N580A, H840A, D1135E, R1335Q, T1337R, T267A, L325F, V327I, D333G, A336S, I341L, E345D, D348N, K352E, S360A, T368A, N369E, N371E, S372P, E373K, K386T, N393R, H408N, N410S, I414M, A415T, T438S, Y467F, N471K, D485E, M489F, E506K, R409K, T510E, N515K, Y518F, A539P, F550Y, N551H, S596A, T602I, A611S, I617V, T620K, G654E, N667D, R685K, K695Q, I706V, K722T, A723T, K724N, M73IT, F732V, K735Q, S739N, P741L, E742G, E746D, Q747D, I754D, T755I, H757R, K760Q, H761S, P778I, E781K, I783V, N784D, D785E, T786L, L787V, Y788H, K792E, D794T, T798R, L799I, V801I, N803S, L804I, N805K, G806N, D813G, K814E, L8181, 1819F, S822P, E824G, L841T, G847S, D848N, Y857H, V8751, 1876V, N884K, A888V, L890R, D894G, D895H, P897L, V903I, G920D, F924L, N929Y, E936D, N937G, V941I, N942D, S943L, C945A, E947K, K951R, L952Q, S956N, N957E, Q958K, A959S, N974D, G975K, V983A, N984S, N985D, D986G, I991V, V993L, M995F, I996V, T999N, Y1000K, R1001E, E1002D, L1004I, E1005K, N1006M, M1007N, D1009L, K1010S, R1011T, P1012S, P1013F, I1015L, I1016R, A1020G, 51021K, Q1024K, K1027S, E1039K, H1045K, I0148M, K1050M or a combination thereof. In some embodiments, the RNA-guided nuclease Staphylococcus aureus Cas9 domain comprises an amino acid sequence that is at least 85%, 90%, 95%, 97%, 98%, or 99% identical to SEQ ID NO: 710. In some embodiments, the RNA-guided nuclease Staphylococcus aureus Cas9 domain comprises a mutation corresponding to any one of positions D10, H557, N580, H840, D1135, R1335, T1337, T267, L325, V327, D333, A336, 1341, E345, D348, K352, S360, T368, N369, N371, S372, E373, K386, N393, H408, N410, 1414, A415, T438, Y467, N471, D485, M489, E506, R409, T510, N515, Y518, A539, F550, N551, S596, T602, A611, I617, T620, G654, N667, R685, K695, 1706, K722, A723, K724, M731, F732, K735, S739, P741, E742, E746, Q747, 1754, T755, H757, K760, H761, P778, E781, 1783, N784, D785, T786L, L787, Y788, K792, D794, T798, L799, V801, N803, L804, N805, G806, D813, K814, L818, 1819, S822, E824, L841, G847, D848, Y857, V875, 1876, N884, A888, L890, D894, D895, P897, V903, G920, F924, N929, E936, N937, V941, N942, S943, C945, E947, K951, L952, S956, N957, Q958, A959, N974, G975, V983, N984, N985, D986, I991, V993, M995, I996, T999, Y1000, R1001, E1002, L1004, E1005, N1006, M1007, D1009, K1010, R1011, P1012, P1013, I1015, 11016, A1020, S1021, Q1024, K1027, E1039, H1045, 10148, K1050 or a combination thereof. In some embodiments, the RNA-guided nuclease Staphylococcus aureus Cas9 domain comprises a mutation selected from a mutation corresponding to any one of D10A, D10E, H557A, N580A, H840A, D1135E, R1335Q, T1337R, T267A, L325F, V327I, D333G, A336S, I341L, E345D, D348N, K352E, S360A, T368A, N369E, N371E, S372P, E373K, K386T, N393R, H408N, N410S, I414M, A415T, T438S, Y467F, N471K, D485E, M489F, E506K, R409K, T510E, N515K, Y518F, A539P, F550Y, N551H, S596A, T602I, A611S, I617V, T620K, G654E, N667D, R685K, K695Q, I706V, K722T, A723T, K724N, M73IT, F732V, K735Q, S739N, P741L, E742G, E746D, Q747D, I754D, T755I, H757R, K760Q, H761S, P778I, E781K, I783V, N784D, D785E, T786L, L787V, Y788H, K792E, D794T, T798R, L799I, V801I, N803S, L804I, N805K, G806N, D813G, K814E, L8181, 1819F, S822P, E824G, L841T, G847S, D848N, Y857H, V8751, 1876V, N884K, A888V, L890R, D894G, D895H, P897L, V903I, G920D, F924L, N929Y, E936D, N937G, V941I, N942D, S943L, C945A, E947K, K951R, L952Q, S956N, N957E, Q958K, A959S, N974D, G975K, V983A, N984S, N985D, D986G, I991V, V993L, M995F, I996V, T999N, Y1000K, R1001E, E1002D, L1004I, E1005K, N1006M, M1007N, D1009L, K1010S, R1011T, P1012S, P1013F, I1015L, I1016R, A1020G, 51021K, Q1024K, K1027S, E1039K, H1045K, I0148M, K1050M or a combination thereof. In some embodiments, the RNA-guided nuclease Staphylococcus aureus Cas9 domain comprises a mutation corresponding to the D10E mutation. In some embodiments, the RNA-guide nuclease Cas9 domain is an RNA-guided nuclease Streptococcus pyogenes Cas9 domain. In some embodiments, the RNA-guided nuclease Streptococcus pyogenes Cas9 domain comprises an amino acid sequence as set forth in SEQ ID NO: 711. In some embodiments, the RNA-guided nuclease Streptococcus pyogenes Cas9 domain comprises a mutation corresponding to any one of positions D10, S29, F32, D39, R40, H41, S42, I48, C80, S87, K112, H113, K132, K141, D147, L158, E171, P176, I186, V189, Q190, Q194, N199, 1201, N202, A203, S204, R205, A210, Q228, L229, G231, S245, T249, S254, D261, T270, N295, T300, D304, V308, N309, I312, T333, A337, E345, F352, Q354, S355, K356, G366, A367, E396, L398, 1414, D428, F429, D435, K468, S469, E470, T472, E480, A486, S490, F498, K500, N501, N504, K528, V530, E532, G533, A538, T555, K570, F575, D605, E611, R629, E634, T638, R655, R664, R671, K705, E706, Q709, K710, S714, G7115, G717, H721, H723, A725, N726, V743, L747, V748, K772, K775, N776, 1788, G792, K797, Y799, T804, N808, L811, R820, N831, R832, V842, L847, N869, E874, N881, Q885, N888, T893, L911, Y945, D946, L949, E952, A1023, Y1036, G1067, G1077, R1078, N1093, R1114, N1115, D1117, A1121, D1125, P1128, K1129, V1146, S1154, S1159, L1164, S1172, N1177, P1178, I1179, D1180, K1211, M1213, G1218, N1234, E1243, K1244, E1253, E1260, K1263, H1264, E1271, Q1272, E1275, V1290, L1291, S1292, A1293, N1295, H1297, R1298, D1299, K1300, R1303, E1307, N1308, I1309, I1310, H1311, L1312, L1315, T1316, N1317, Y1326, D1328, V1342, A1345, I1360, S1363, or a combination thereof. In some embodiments, the RNA-guided nuclease Streptococcus pyogenes Cas9 domain comprises a mutation selected from a mutation corresponding to any one of D10E, D10A, S29T, F32M, D39N, R40K, H41Q, S42T, I48L, C80R, S87A, K112D, H113N, K132N, K141E, D147E, L158V, E171Q, P176S, I186K, V189L, Q190H, Q194E, N199R, I201L, N202E, A203E, S204I, R205K, A210G, Q228A, L229F, G23I N, S245A, T249M, S254A, D261N, T270S, N295K, T300I, D304G, V308A, N309D, I312V, T333A, A337V, E345K, F352S, Q354K, S355T, K356T, G366K, A367T, E396D, L398F, I414V, D428A, F429Y, D435E, K468Q, S469R, E470N, T472A, E480D, A486T, S490L, F498V, K500E, N501H, N504T, K528R, V530I, E532D, G533E, A538E, T555A, K570Q, F575C, D605E, E611D, R629K, E634K, T638K, R655H, R664K, R671K, K705V, E706D, Q709K, K710A, S714F, G7115E, G717K, H721K, H723Q, A725S, N726A, V743I, L747I, V748I, K772Q, K775R, N776R, I788M, G792R, K797E, Y799H, T804A, N808D, L811R, R820K, N83I D, R832H, V842I, L847I, N869D, E874A, N881S, Q885R, N888K, T893S, L91I A, Y945H, D946G, L949P, E952A, A1023G, Y1036R, G1067E, G1077E, R1078K, N1093T, R1114G, N1115E, D1117A, A1121P, D1125G, P1128T, K1129T, V11461, S1154T, S1159P, L1164V, S1172N, N1177D, P1178S, 11179V, D1180S, K1211R, M1213L, G1218T, N1234H, E1243D, K1244T, E1253K, E1260D, K1263Q, H1264Y, E1271D, Q1272W, E1275H, V1290L, L1291R, S1292A, A1293T, N1295E, H1297N, R1298T, D1299H, K1300L, R1303S, E1307D, N1308S, I1309M, I1310L, H1311N, L1312A, L1315F, T1316S, N1317R, Y1326F, D1328N, V1342I, A1345S, I1360L, S1363N, or a combination thereof. In some embodiments, the RNA-guided nuclease Streptococcus pyogenes Cas9 domain comprises an amino acid sequence that is at least 85%, 90%, 95%, 97%, 98%, or 99% identical to SEQ ID NO: 711. In some embodiments, the RNA-guided nuclease Staphylococcus pyogenes Cas9 domain comprises a mutation corresponding to any one of positions D10, S29, F32, D39, R40, H41, S42, I48, C80, S87, K112, H113, K132, K141, D147, L158, E171, P176, I186, V189, Q190, Q194, N199, 1201, N202, A203, S204, R205, A210, Q228, L229, G231, S245, T249, S254, D261, T270, N295, T300, D304, V308, N309, I312, T333, A337, E345, F352, Q354, S355, K356, G366, A367, E396, L398, 1414, D428, F429, D435, K468, 5469, E470, T472, E480, A486, S490, F498, K500, N501, N504, K528, V530, E532, G533, A538, T555, K570, F575, D605, E611, R629, E634, T638, R655, R664, R671, K705, E706, Q709, K710, S714, G7115, G717, H721, H723, A725, N726, V743, L747, V748, K772, K775, N776, 1788, G792, K797, Y799, T804, N808, L811, R820, N831, R832, V842, L847, N869, E874, N881, Q885, N888, T893, L911, Y945, D946, L949, E952, A1023, Y1036, G1067, G1077, R1078, N1093, R1114, N1115, D1117, A1121, D1125, P1128, K1129, V1146, S1154, 51159, L1164, 51172, N1177, P1178, I1179, D1180, K1211, M1213, G1218, N1234, E1243, K1244, E1253, E1260, K1263, H1264, E1271, Q1272, E1275, V1290, L1291, S1292, A1293, N1295, H1297, R1298, D1299, K1300, R1303, E1307, N1308, I1309, I1310, H1311, L1312, L1315, T1316, N1317, Y1326, D1328, V1342, A1345, I1360, S1363, or a combination thereof. In some embodiments, the RNA-guided nuclease Streptococcus pyogenes Cas9 domain comprises a mutation selected from a mutation corresponding to any one of D10E, D10A, S29T, F32M, D39N, R40K, H41Q, S42T, I48L, C80R, S87A, K112D, Hi 13N, K132N, K141E, D147E, L158V, E171Q, P176S, I186K, V189L, Q190H, Q194E, N199R, I201L, N202E, A203E, S204I, R205K, A210G, Q228A, L229F, G23I N, S245A, T249M, S254A, D261N, T270S, N295K, T300I, D304G, V308A, N309D, I312V, T333A, A337V, E345K, F352S, Q354K, S355T, K356T, G366K, A367T, E396D, L398F, I414V, D428A, F429Y, D435E, K468Q, S469R, E470N, T472A, E480D, A486T, S490L, F498V, K500E, N501H, N504T, K528R, V530I, E532D, G533E, A538E, T555A, K570Q, F575C, D605E, E611D, R629K, E634K, T638K, R655H, R664K, R671K, K705V, E706D, Q709K, K710A, S714F, G7115E, G717K, H721K, H723Q, A725S, N726A, V743I, L747I, V748I, K772Q, K775R, N776R, I788M, G792R, K797E, Y799H, T804A, N808D, L811R, R820K, N83I D, R832H, V842I, L847I, N869D, E874A, N881S, Q885R, N888K, T893S, L911A, Y945H, D946G, L949P, E952A, A1023G, Y1036R, G1067E, G1077E, R1078K, N1093T, R1114G, N1115E, D1117A, A1121P, D1125G, P1128T, K1129T, V11461, S1154T, S1159P, L1164V, S1172N, N1177D, P1178S, 11179V, D1180S, K1211R, M1213L, G1218T, N1234H, E1243D, K1244T, E1253K, E1260D, K1263Q, H1264Y, E1271D, Q1272W, E1275H, V1290L, L1291R, S1292A, A1293T, N1295E, H1297N, R1298T, D1299H, K1300L, R1303S, E1307D, N1308S, I1309M, I1310L, H1311N, L1312A, L1315F, T1316S, N1317R, Y1326F, D1328N, V1342I, A1345S, 11360L, S1363N, or a combination thereof. In some embodiments, the RNA-guided nuclease Cas9 domain is an RNA-guided nuclease Neisseria meningitidis Cas9 domain. In some embodiments, the RNA-guided nuclease Neisseria meningitidis Cas9 domain comprises an amino acid sequence as set forth in SEQ ID NO: 712. In some embodiments, the RNA-guided nuclease Neisseria meningitidis Cas9 domain comprises a mutation corresponding to any one of positions I9, D16, D30, E31, A94, I103, P124, N164, I213, G229, T241, 5376, E393, G454, K471, G490, D660, C665, K764, T770, P803, A841, H842, K843, D844, L846, R847, K854, H855, N856, K858, K862, W865, E868, 1869, A872, D873, N876, Y880, G883, 1886, E887, E890, R895, A898, Y899, G900, G901, N902, A903, K904, Q905, D908, N912, K917, G919, L921, V927, K929, T930, E932, S933, L936, L937, N938, K939, K940, Y943, T944, G949, D950, C958, K965, N966, Q967, F969, A975, E980, N981, I986, D987, C988, K989, G990, Y991, R992, I993, D994, Y997, T998, C1000, S1002, H1004, K1005, Y1006, A1010, F1011, Q1012, K1013, D1014, E1015, K1018, V1019, E1020, F1021, A1022, Y1024, I1025, N1026, C1027, D1028, S1029, S1030, N1031, R1033, F1034, Y1035, L1036, A1037, W1038, K1041, G1042, K1044, E1045, Q1046, Q1047, F1048, R1049, I1050, S1051, T1052, Q1053, N1054, L1055, V1056, L1057, I1058, Y1061, V1063, N1064, or a combination thereof. In some embodiments, the RNA-guided nuclease Neisseria meningitidis Cas9 domain comprises a mutation selected from a mutation corresponding to any one of I9M, D16E, D30E, E31K, A94D, I103V, P124C, N164D, I213N, G229D, T241A, S376T, E393K, G454C, K471E, G490C, D660E, C665R, K764E, T770A, P803S, A841Q, H842G, K843H, D844E, L846V, R847K, K854R, H855L, N856D, K858G, K862L, W865P, E868Q, I869L, A872K, D873G, N876K, Y880R, G883E, I886P, E887K, E890E, R895Q, A898T, Y899H, G900K, G901D, N902D, A903P, K904T, Q905K, D908A, N912E, K917Y, G919T, L921Q, V927I, K929Q, T930V, E932K, S933T, L936W, L937V, N938R, K939N, K940H, Y943N, T944G, G949A, D950T, C958E, K965G, N966G, Q967K, F969Y, A975S, E980K, N981G, I986R, D987A, C988V, K989V, G990A, Y991F, R992K, I993D, D994E, Y997F, T998E, C1000R, 51002I, H1004Y, K1005A, Y1006N, A1010K, F1011L, Q1012T, K1013A, D1014K, E1015K, K1018N, V1019E, E1020F, F1021L, A1022G, Y1024F, I1025V, N1026S, C1027L, D1028N, S1029R, S1030A, N103IT, R1033A, F1034I, Y1035D, L1036I, A1037R, W1038T, K1041T, G1042D, K1044T, E1045K, Q1046G, Q1047E, F1048Q, R1049S, I1050V, S1051G, T1052V, Q1053K, N1054T, L1055A, V1056L, L1057S, I1058F, Y1061N, V1063I, N1064D, or a combination thereof. In some embodiments, the RNA-guided nuclease Neisseria meningitidis Cas9 domain comprises an amino acid sequence that is at least 85%, 90%, 95%, 97%, 98%, or 99% identical to SEQ ID NO: 712. In some embodiments, the RNA-guided nuclease Neisseria meningitidis Cas9 domain comprises a mutation corresponding to any one of positions 19, D16, D30, E31, A94, I103, P124, N164, I213, G229, T241, S376, E393, G454, K471, G490, D660, C665, K764, T770, P803, A841, H842, K843, D844, L846, R847, K854, H855, N856, K858, K862, W865, E868, 1869, A872, D873, N876, Y880, G883, 1886, E887, E890, R895, A898, Y899, G900, G901, N902, A903, K904, Q905, D908, N912, K917, G919, L921, V927, K929, T930, E932, S933, L936, L937, N938, K939, K940, Y943, T944, G949, D950, C958, K965, N966, Q967, F969, A975, E980, N981, I986, D987, C988, K989, G990, Y991, R992, I993, D994, Y997, T998, C1000, S1002, H1004, K1005, Y1006, A1010, F1011, Q1012, K1013, D1014, E1015, K1018, V1019, E1020, F1021, A1022, Y1024, I1025, N1026, C1027, D1028, S1029, S1030, N1031, R1033, F1034, Y1035, L1036, A1037, W1038, K1041, G1042, K1044, E1045, Q1046, Q1047, F1048, R1049, I1050, S1051, T1052, Q1053, N1054, L1055, V1056, L1057, I1058, Y1061, V1063, N1064, or a combination thereof. In some embodiments, the RNA-guided nuclease Neisseria meningitidis Cas9 domain comprises a mutation selected from a mutation corresponding to any one of I9M, D16E, D30E, E31K, A94D, I103V, P124C, N164D, I213N, G229D, T241A, S376T, E393K, G454C, K471E, G490C, D660E, C665R, K764E, T770A, P803S, A841Q, H842G, K843H, D844E, L846V, R847K, K854R, H855L, N856D, K858G, K862L, W865P, E868Q, I869L, A872K, D873G, N876K, Y880R, G883E, I886P, E887K, E890E, R895Q, A898T, Y899H, G900K, G901D, N902D, A903P, K904T, Q905K, D908A, N912E, K917Y, G919T, L921Q, V927I, K929Q, T930V, E932K, S933T, L936W, L937V, N938R, K939N, K940H, Y943N, T944G, G949A, D950T, C958E, K965G, N966G, Q967K, F969Y, A975S, E980K, N981G, I986R, D987A, C988V, K989V, G990A, Y991F, R992K, I993D, D994E, Y997F, T998E, C1000R, S1002I, H1004Y, K1005A, Y1006N, A1010K, F1011L, Q1012T, K1013A, D1014K, E1015K, K1018N, V1019E, E1020F, F1021L, A1022G, Y1024F, I1025V, N1026S, C1027L, D1028N, S1029R, S1030A, N1031T, R1033A, F1034I, Y1035D, L10361, A1037R, W1038T, K1041T, G1042D, K1044T, E1045K, Q1046G, Q1047E, F1048Q, R1049S, I1050V, S1051G, T1052V, Q1053K, N1054T, L1055A, V1056L, L1057S, I1058F, Y1061N, V1063I, N1064D, or a combination thereof. In some embodiments, the RNA-guide nuclease Cas9 domain is an RNA-guided nuclease Campylobacter jejuni Cas9 domain. In some embodiments, the RNA-guided nuclease Campylobacterjejuni Cas9 domain comprises an amino acid sequence as set forth in SEQ ID NO: 713. In some embodiments, the RNA-guided nuclease Campylobacter jejuni Cas9 domain comprises a mutation corresponding to any one of positions L5, A6, D8, I9, S12, S13, F18, S19, L24, K25, 131, T40, E42, L50, L58, A59, R61, L58, L65, H67AN74, K77, L98, I99, P101, N110, L113, A119, A126, R128, I134, K140, A144, K147, Q151, L156, V184, S190, F199, D202, G203, R212, F214, K221, E223, Y232, A235, V243, 5247, D251, P256, L261, T269, N276, N277, L285, T287, L291, K300, T305, Q308, L312, G314, Y335, K336, I339, H345, D351, N353, E354, 1362, K370, D383E, S384, K391, 1396, L403, T405, K413, N419, L421, D430, K432, A437, L453, K457, V462, A465, K472, N477, A492, E495, L525, K526, L527, K531, E532, E542, Q550, E556, H559, Y561, 5564, M572, V577, Q581, N587, N596, K600, Q602, K603, Q616, K617, N623, Y624, K633, D634, Y642, N649, D656, L660, D662, K667, V677, E680, K682, L686, H692, T693, V712, I714, V722, K723, 5736, L739, K742, L747, N751, F756, R763, Q764, E772, K777, A786, E790, F792, Q800, S801, G804, L812, E813, V833, 1835, T841, Y845, A855, L856, A863, V864, D879, E883, D900, Q902, K927, F928, V971, T972, or a combination thereof. In some embodiments, the RNA-guided nuclease Campylobacter jejuni Cas9 domain comprises a mutation selected from a mutation corresponding to any one of L5I, A6G, D8N, D8E, I9L, S12A, S13N, F18L, S19R, L24I, K25I, 131V, T40N, E42N, L50E, L58V, A59K, R61K, L58V, L65M, H67A, N74K, K77N, L98T, I99Q, P101I, N110S, L113I, A119S, A126V, R128H, I134S, K140N, A144T, K147E, Q151K, L156M, V184I, S190D, F199L, D202Q, G203E, R212K, F214L, K221K, E223K, Y232F, A235P, V243I, S247I, D251N, P256A, L261S, T269G, N276K, N277S, L285V, T287E, L2911, K300D, T305S, Q308K, L312I, G314N, Y335L, K336N, I339K, H345T, D351I, N353D, E354S, I362T, K370E, D383E, S384K, K391N, I396L, L403Q, T405I, K413R, N419E, L421C, D430E, K432S, A437L, L453I, K457C, V462L, A465D, K472S, N477H, A492K, E495I, L525Q, K526I, L527V, K531E, E532D, E542L, Q550D, E556V, H559Y, Y561R, S564N, M572S, V577T, Q581L, N587G, N596E, K600L, Q602A, K603E, Q616R, K617F, N623F, Y624F, K633T, D634E, Y642W, N649S, D656S, L660I, D662E, K667A, V677Q, E680V, K682S, L686I, H692N, T693F, V7121, I714V, V722I, K723F, S736K, L739F, K742N, L747S, N751L, F756L, R763K, Q764E, E772N, K777H, A786T, E790L, F792P, Q800N, S801T, G804D, L812V, E813K, V833S, I835L, T841K, Y845H, A855S, L856T, A863T, V864P, D879N, E883N, D900G, Q902K, K927N, F928Y, V971L, T972S, or a combination thereof. In some embodiments, the RNA-guided nuclease Campylobacter jejuni Cas9 domain comprises an amino acid sequence that is at least 85%, 90%, 95%, 97%, 98%, or 99% identical to SEQ ID NO: 713. In some embodiments, the RNA-guided nuclease Campylobacter jejuni Cas9 domain comprises a mutation corresponding to any one of positions L5, A6, D8, I9, S12, S13, F18, S19, L24, K25, 131, T40, E42, L50, L58, A59, R61, L58, L65, H67A N74, K77, L98, I99, P101, N110, L113, A119, A126, R128, I134, K140, A144, K147, Q151, L156, V184, S190, F199, D202, G203, R212, F214, K221, E223, Y232, A235, V243, 5247, D251, P256, L261, T269, N276, N277, L285, T287, L291, K300, T305, Q308, L312, G314, Y335, K336, 1339, H345, D351, N353, E354, 1362, K370, D383E, 5384, K391, 1396, L403, T405, K413, N419, L421, D430, K432, A437, L453, K457, V462, A465, K472, N477, A492, E495, L525, K526, L527, K531, E532, E542, Q550, E556, H559, Y561, 5564, M572, V577, Q581, N587, N596, K600, Q602, K603, Q616, K617, N623, Y624, K633, D634, Y642, N649, D656, L660, D662, K667, V677, E680, K682, L686, H692, T693, V712, I714, V722, K723, 5736, L739, K742, L747, N751, F756, R763, Q764, E772, K777, A786, E790, F792, Q800, S801, G804, L812, E813, V833, 1835, T841, Y845, A855, L856, A863, V864, D879, E883, D900, Q902, K927, F928, V971, T972, or a combination thereof. In some embodiments, the RNA-guided nuclease Campylobacter jejuni Cas9 domain comprises a mutation selected from a mutation corresponding to any one of L5I, A6G, D8N, D8E, I9L, S12A, S13N, F18L, S19R, L24I, K25I, 131V, T40N, E42N, L50E, L58V, A59K, R61K, L58V, L65M, H67A, N74K, K77N, L98T, I99Q, P101I, N110S, L113I, A119S, A126V, R128H, I134S, K140N, A144T, K147E, Q151K, L156M, V184I, S190D, F199L, D202Q, G203E, R212K, F214L, K221K, E223K, Y232F, A235P, V243I, S247I, D251N, P256A, L261S, T269G, N276K, N277S, L285V, T287E, L291I, K300D, T305S, Q308K, L312I, G314N, Y335L, K336N, I339K, H345T, D351I, N353D, E354S, I362T, K370E, D383E, S384K, K391N, I396L, L403Q, T405I, K413R, N419E, L421C, D430E, K432S, A437L, L453I, K457C, V462L, A465D, K472S, N477H, A492K, E495I, L525Q, K526I, L527V, K531E, E532D, E542L, Q550D, E556V, H559Y, Y561R, S564N, M572S, V577T, Q581L, N587G, N596E, K600L, Q602A, K603E, Q616R, K617F, N623F, Y624F, K633T, D634E, Y642W, N649S, D656S, L660I, D662E, K667A, V677Q, E680V, K682S, L686I, H692N, T693F, V7121, I714V, V722I, K723F, S736K, L739F, K742N, L747S, N751L, F756L, R763K, Q764E, E772N, K777H, A786T, E790L, F792P, Q800N, S801T, G804D, L812V, E813K, V833S, I835L, T841K, Y845H, A855S, L856T, A863T, V864P, D879N, E883N, D900G, Q902K, K927N, F928Y, V971L, T972S, or a combination thereof. In some embodiments, the RNA-guide nuclease Cas9 domain is an RNA-guided nuclease Streptococcus pasteurianus Cas9 domain. In some embodiments, the RNA-guided nuclease Streptococcus pasteurianus Cas9 domain comprises an amino acid sequence as set forth in SEQ ID NO: 714. In some embodiments, the RNA-guided nuclease Streptococcus pasteurianus Cas9 domain comprises a mutation corresponding to any one of positions D11, E85, A88, T92, E96, Y100, T109, D110, D113, E115, R116, D125, I127, K128, E132, S147, I185, A187, K228, Y229, T232, M255, S271, N273, A294, A327, E355, K357, N379, T380, S382, A385, D439, R440, S464, H469, Y519, I528, N569, I581, A607, K632, D633, H635, E636, A647, D648, T703, P705, K712, S713, A724, V750, D882, S951, D977, E979, S1014, H1027, I1030, E1081, D1082, D1086, K1088, S1089, N1090, R1092, T1093, I1094, C1095, A1138, Y1139, D1141, T1142, F1158, A1168, E1190, E1198, H1202, I1204, R1205, I1210, K1224, S1232, M1240, V1241, I1242, P1243, G1424, K1248, Q1254, N1257, S1258, T1262, K1263, Y1264, D1266, A1270, K1277, D1284, L1288, V1302, N1316, T1346, I1374, or a combination thereof. In some embodiments, the RNA-guided nuclease Streptococcus pasteurianus Cas9 domain comprises a mutation selected from a mutation corresponding to any one of D11E, D11A, E85D, A88T, T92A, E96D, Y100Q, T109D, D110N, D113N, E115D, R116S, D125E, I127D, K128A, E132K, S147T, I185L, A187T, K228N, Y229N, T232K, M255T, S271T, N273E, A294S, A327V, E355K, K357Q, N379G, T380I, S382T, A385N, D439E, R440E, S464A, H469R, Y519F, I528V, N569D, I581V, A607S, K632R, D633E, H635Q, E636Q, A647K, D648Q, T703A, P705S, K712E, S713A, A724T, V750I, D882G, S951R, D977E, E979K, S1014P, H1027R, I1030V, E1081G, D1082E, D1086N, K1088R, S1089T, N1090D, R1092E, T1093K, I1094V, C1095R, A1138V, Y1139L, D1141E, T1142P, F1158L, A1168T, E1190K, E1198K, H1202Q, I1204V, R1205Q, I1210M, K1224R, S1232T, M1240I, V1241M, I1242L, P1243S, G1424A, K1248A, Q1254H, N1257G, S1258N, T1262A, K1263E, Y1264H, D1266K, A1270E, K1277E, D1284N, L1288V, V1302A, N1316D, T1346N, I1374L, or a combination thereof. In some embodiments, the RNA-guided nuclease Streptococcus pasteurianus Cas9 domain comprises an amino acid sequence that is at least 85%, 90%, 95%, 97%, 98%, or 99% identical to SEQ ID NO: 714. In some embodiments, the RNA-guided nuclease Streptococcus pasteurianus Cas9 domain comprises a mutation corresponding to any one of positions D11, E85, A88, T92, E96, Y100, T109, D110, D113, E115, R116, D125, I127, K128, E132, S147, I185, A187, K228, Y229, T232, M255, S271, N273, A294, A327, E355, K357, N379, T380, S382, A385, D439, R440, S464, H469, Y519, I528, N569, I581, A607, K632, D633, H635, E636, A647, D648, T703, P705, K712, S713, A724, V750, D882, S951, D977, E979, S1014, H1027, I1030, E1081, D1082, D1086, K1088, S1089, N1090, R1092, T1093, I1094, C1095, A1138, Y1139, D1141, T1142, F1158, A1168, E1190, E1198, H1202, I1204, R1205, I1210, K1224, S1232, M1240, V1241, I1242, P1243, G1424, K1248, Q1254, N1257, S1258, T1262, K1263, Y1264, D1266, A1270, K1277, D1284, L1288, V1302, N1316, T1346, I1374, or a combination thereof. In some embodiments, the RNA-guided nuclease Streptococcus pasteurianus Cas9 domain comprises a mutation selected from a mutation corresponding to any one of D11E, D11A, E85D, A88T, T92A, E96D, Y100Q, T109D, D110N, D113N, E115D, R116S, D125E, I127D, K128A, E132K, S147T, I185L, A187T, K228N, Y229N, T232K, M255T, S271T, N273E, A294S, A327V, E355K, K357Q, N379G, T380I, S382T, A385N, D439E, R440E, S464A, H469R, Y519F, I528V, N569D, I581V, A607S, K632R, D633E, H635Q, E636Q, A647K, D648Q, T703A, P705S, K712E, S713A, A724T, V750I, D882G, S951R, D977E, E979K, S1014P, H1027R, I1030V, E1081G, D1082E, D1086N, K1088R, S1089T, N1090D, R1092E, T1093K, I1094V, C1095R, A1138V, Y1139L, D1141E, T1142P, F1158L, A1168T, E1190K, E1198K, H1202Q, I1204V, R1205Q, I1210M, K1224R, S1232T, M1240I, V1241M, I1242L, P1243S, G1424A, K1248A, Q1254H, N1257G, S1258N, T1262A, K1263E, Y1264H, D1266K, A1270E, K1277E, D1284N, L1288V, V1302A, N1316D, T1346N, I1374L, or a combination thereof. In some embodiments, the RNA-guide nuclease Cas9 domain is an RNA-guided nuclease Clostridium cellulolyticum Cas9 domain. In some embodiments, the RNA-guided nuclease Clostridium cellulolyticum Cas9 domain comprises an amino acid sequence as set forth in SEQ ID NO: 715. In some embodiments, the RNA-guided nuclease Clostridium cellulolyticum Cas9 domain comprises a mutation corresponding to any one of positions T4, D10, V9, D20, K21, 127, C33, K36, A47, A49, S64, Q65, E102, L103, T122, I1124, K131, D137, R163, G166, I1169, F170, V183, D184, I187, E193, K200, K208, L209, D221, N224, E227, F228, 5234, V242, K244, L252, T256, C258, 5261, V413, M415, K416, R417, K424, Y426, K427, S429, D430, A468, T470, A472, A478, Q481, K482, L485, A497, L535, W540, R541, E544, G554, P556, I1570, Y574, M580, Y584, M585, T592, D593, V606, W607, I647, N650, S693, L697, E702, S704, A713, V714, I1715, D776, L847, G850, G853, A854, R860, I900, H904, M905, I906, E921, Q923, S929, T930, H931, Q939, N994, I997, N1000, K1001, S1002, I1003, K1005, P1008, or a combination thereof. In some embodiments, the RNA-guided nuclease Clostridium cellulolyticum Cas9 domain comprises a mutation selected from any one of T4S, D10E, V9I, D20N, K21E, 127E, C33I, K36V, A47S, A49P, S64R, Q65H, E102L, L103V, T122V, I124F, K131Q, D137E, R163Q, G166S, I169L, F170L, V183G, D184G, I187T, E193S, K200Q, K208A, L209Y, D221K, N224Q, E227S, F228S, S234T, V242I, K244N, L252K, T256K, C258T, S261F, V413K, M415L, K416R, R417N, K424Q, Y426I, K427P, S429H, D430Q, A468S, T470S, A472V, A478G, Q481K, K482R, L485S, A497M, L535H, W540Y, R541K, E544Q, G554F, P556S, I570V, Y574I, M580F, Y584N, M585N, T592A, D593A, V606W, W607F, I647R, N650H, S693K, L697F, E702Q, S704N, A713V, V7141, I1715V, D776E, L847A, G850P, G853A, A854P, R860K, I900V, H904D, M905V, I906L, E921Y, Q923E, S929D, T930E, H931Y, Q939P, N994Q, I997P, N1000R, K1001M, S1002N, I1003K, K1005H, P1008K or a combination thereof. In some embodiments, the RNA-guided nuclease Clostridium cellulolyticum Cas9 domain comprises an amino acid sequence that is at least 85%, 90%, 95%, 97%, 98%, or 99% identical to SEQ ID NO: 715. In some embodiments, the RNA-guided nuclease Clostridium cellulolyticum Cas9 domain comprises a mutation corresponding to any one of positions T4, D10, V9, D20, K21, 127, C33, K36, A47, A49, S64, Q65, E102, L103, T122, I124, K131, D137, R163, G166, I1169, F170, V183, D184, I187, E193, K200, K208, L209, D221, N224, E227, F228, S234, V242, K244, L252, T256, C258, S261, V413, M415, K416, R417, K424, Y426, K427, S429, D430, A468, T470, A472, A478, Q481, K482, L485, A497, L535, W540, R541, E544, G554, P556, I1570, Y574, M580, Y584, M585, T592, D593, V606, W607, I647, N650, S693, L697, E702, S704, A713, V714, I1715, D776, L847, G850, G853, A854, R860, I900, H904, M905, I906, E921, Q923, S929, T930, H931, Q939, N994, I997, N1000, K1001, S1002, 11003, K1005, P1008, or a combination thereof. In some embodiments, the RNA-guided nuclease Clostridium cellulolyticum Cas9 domain comprises a mutation selected from a mutation corresponding to any one of T4S, D10E, V9I, D20N, K21E, 127E, C33I, K36V, A47S, A49P, S64R, Q65H, E102L, L103V, T122V, I124F, K131Q, D137E, R163Q, G166S, I169L, F170L, V183G, D184G, I187T, E193S, K200Q, K208A, L209Y, D221K, N224Q, E227S, F228S, S234T, V242I, K244N, L252K, T256K, C258T, S261F, V413K, M415L, K416R, R417N, K424Q, Y426I, K427P, S429H, D430Q, A468S, T470S, A472V, A478G, Q481K, K482R, L485S, A497M, L535H, W540Y, R541K, E544Q, G554F, P556S, I570V, Y574I, M580F, Y584N, M585N, T592A, D593A, V606W, W607F, I647R, N650H, S693K, L697F, E702Q, S704N, A713V, V7141, I1715V, D776E, L847A, G850P, G853A, A854P, R860K, I900V, H904D, M905V, I906L, E921Y, Q923E, S929D, T930E, H931Y, Q939P, N994Q, I997P, N1000R, K1001M, S1002N, I1003K, K1005H, P1008K or a combination thereof. In some embodiments, the RNA-guide nuclease Cas9 domain is an RNA-guided nuclease Geobacillus thermodenitrificans T1 Cas9 domain. In some embodiments, the RNA-guided nuclease Geobacillus thermodenitrificans T1 Cas9 domain comprises an amino acid sequence as set forth in SEQ ID NO: 716. In some embodiments, the RNA-guided nuclease Geobacillus thermodenitrificans T1 Cas9 domain comprises a mutation corresponding to any one of positions K2, D8, I14, D35, K41, F74, V75, K91, I117, R128, T136, Q151, S152, S156, A161, V164, S171, E178, D179, V185, R192, K195, A199, Y204, 1207, V208, A212, H215, S219, F227, T260, V261, V271, G274, I276, A278, L279, D282, I287, K289, H293, F299, V302, N307, R313, L317, L318, V331, G337, K341, 5348, A354, A355, K356, R359, M372, T377, R380, E395, D399, E404, S416, T441, R445, N464, E504, S508, M515, Q516, E520, G521, V534, L545, K559, T578, K603, T612, L619, S621, N656, N660, L673, D685, I699, N708, N717, R737, V738, 5752, D756, Q771, N777, N792, E793, 1811, 1824, K839, Q845, K848, T849, L895, I902, T908, V929, I943, I946, M948, F990, T995, V1000, Q1014, D1017, S1019, N1020, G1021, S1024, N1030, N1031, R1035, S1036, I1037, V1067, S1071, A1075, 11079, or a combination thereof. In some embodiments, the RNA-guided nuclease Geobacillus thermodenitrificans T1 Cas9 domain comprises a mutation selected from a mutation corresponding to any one of K2R, D8E, D8A, 114V, D35E, K41Q, F74V, V75I, K91E, I117V, R128K, T136S, Q151R, S152A, S156G, A161G, V164I, S171A, E178G, D179E, V185I, R192H, K195R, A199S, Y204F, I207M, V208S, A212K, H215N, S219T, F227V, T260I, V261A, V271I, G274S, I276A, A278G, L279P, D282E, I287L, K289E, H293Q, F299Y, V302I, N307R, R313Y, L317I, L318V, V331I, G337D, K341Q, S348K, A354K, A355S, K356S, R359L, M372L, T377A, R380H, E395P, D399N, E404N, S416T, T441S, R445K, N464T, E504D, S508T, M515T, Q516K, E520D, G521E, V534M, L545H, K559R, T578V, K603R, T612I, L619V, S621T, N656M, N660S, L673F, D685E, I699V, N708E, N717D, R737K, V738I, S752A, D756E, Q771R, N777H, N792D, E793Q, 1811V, I824V, K839T, Q845K, K848A, T849S, L895P, I902V, T908K, V929V, I943V, I946M, M948I, F990L, T995I, V1000G, Q1014K, D1017H, S1019G, N1020T, G1021A, S1024E, N1030C, N1031S, R1035S, S1036G, I1037V, V1067L, 51071A, A1075T, I1079V, or a combination thereof. In some embodiments, the RNA-guided nuclease Geobacillus thermodenitrificans T1 Cas9 domain comprises an amino acid sequence that is at least 85%, 90%, 95%, 97%, 98%, or 99% identical to SEQ ID NO: 716. In some embodiments, the RNA-guided nuclease Geobacillus thermodenitrifcans T1 Cas9 domain comprises a mutation corresponding to any one of positions K2, D8, I14, D35, K41, F74, V75, K91, I117, R128, T136, Q151, S152, S156, A161, V164, S171, E178, D179, V185, R192, K195, A199, Y204, 1207, V208, A212, H215, S219, F227, T260, V261, V271, G274, 1276, A278, L279, D282, 1287, K289, H293, F299, V302, N307, R313, L317, L318, V331, G337, K341, S348, A354, A355, K356, R359, M372, T377, R380, E395, D399, E404, S416, T441, R445, N464, E504, S508, M515, Q516, E520, G521, V534, L545, K559, T578, K603, T612, L619, S621, N656, N660, L673, D685, I699, N708, N717, R737, V738, S752, D756, Q771, N777, N792, E793, 1811, 1824, K839, Q845, K848, T849, L895, I902, T908, V929, I943, I946, M948, F990, T995, V1000, Q1014, D1017, S1019, N1020, G1021, S1024, N1030, N1031, R1035, S1036, I1037, V1067, S1071, A1075, I1079, or a combination thereof. In some embodiments, the RNA-guided nuclease Geobacillus thermodenitrificans T1 Cas9 domain comprises a mutation selected from a mutation corresponding to any one of K2R, D8E, D8A, I14V, D35E, K41Q, F74V, V75I, K91E, I117V, R128K, T136S, Q151R, S152A, S156G, A161G, V164I, S171A, E178G, D179E, V185I, R192H, K195R, A199S, Y204F, I207M, V208S, A212K, H215N, S219T, F227V, T260I, V261A, V271I, G274S, I276A, A278G, L279P, D282E, I287L, K289E, H293Q, F299Y, V302I, N307R, R313Y, L3171, L318V, V331I, G337D, K341Q, S348K, A354K, A355S, K356S, R359L, M372L, T377A, R380H, E395P, D399N, E404N, S416T, T441S, R445K, N464T, E504D, S508T, M515T, Q516K, E520D, G521E, V534M, L545H, K559R, T578V, K603R, T612I, L619V, S621T, N656M, N660S, L673F, D685E, I699V, N708E, N717D, R737K, V738I, S752A, D756E, Q771R, N777H, N792D, E793Q, 1811V, I824V, K839T, Q845K, K848A, T849S, L895P, I902V, T908K, V929V, I943V, I946M, M948I, F990L, T995I, V1000G, Q1014K, D1017H, S1019G, N1020T, G1021A, S1024E, N1030C, N1031S, R1035S, S1036G, I1037V, V1067L, S1071A, A1075T, 11079V, or a combination thereof. In some embodiments, the RNA-guided nuclease Cas domain is a RNA-guided nuclease Cas12 domain. In some embodiments, the RNA-guided nuclease Cas domain is a RNA-guided nuclease CasX domain. In some embodiments, the I-TEVI nuclease domain comprises an amino acid sequence that is at least 85%, 90%, 95%, 97%, 98%, or 99% identical to SEQ ID NO: 700. In some embodiments, the I-TEVI nuclease domain comprises a mutation at any one of positions corresponding to T11, V16, N14, E25, K26, R27, E36, K37, G38, C39, S41, L45, F49, I60, and E81, or a combination thereof. In some embodiments, the I-TEVI nuclease domain comprises a mutation selected from any one of corresponding to T11V, V161, N14G, E25D, K26R, R27A, E36S, K37N, G38N, C39V, S41H, L45F, F49Y, 160V, E811, or a combination thereof. In some embodiments, the I-TEVI nuclease domain comprises a mutation corresponding to a K26R mutation. In some embodiments, the I-TEVI nuclease domain comprises an amino acid sequence as set forth in SEQ ID NO: 700. In some embodiments, the I-TEVI nuclease domain comprises a mutation corresponding to any one of positions T11, V16, N14, E25, K26, R27, E36, K37, G38, C39, S41, L45, F49, I60, and E81, or a combination thereof. In some embodiments, the I-TEVI nuclease domain comprises a mutation selected from a mutation corresponding to any one of TiiV, V16I, N14G, E25D, K26R, R27A, E36S, K37N, G38N, C39V, S41H, L45F, F49Y, I60V, E811, or a combination thereof. In some embodiments, the I-TEVI nuclease domain comprises a mutation corresponding to a K26R mutation. In some embodiments, the chimeric nuclease further comprises a nuclear localization signal. In some embodiments, the nuclear localization signal comprises an SV40 nuclear localization signal. In some embodiments, the nuclear localization signal comprises a Nucleoplasmin nuclear localization signal. In some embodiments, the composition further comprises a donor nucleic acid. In some embodiments, the donor nucleic acid restores a non-oncogenic function of a gene comprising the oncogenic mutation. In some embodiments, the donor nucleic acid comprises a non-oncogenic version of the oncogenic mutation. In some embodiments, the donor nucleic acid is DNA. In some embodiments, the donor nucleic acid comprises a blunt end and at least two nucleotide 3′ overhang end. In some embodiments, the donor nucleic acid comprises a 5′ and a 3′ homology flanking the non-oncogenic version of the oncogenic mutation. In some embodiments, the composition does not comprise a donor nucleic acid. In some embodiments, the composition further comprises a pharmaceutically acceptable excipient, diluent or carrier. In some embodiments, the composition is encapsulated in a lipid nanoparticle. In some embodiments, the lipid nanoparticle comprises cationic or neutral lipids.

In another aspect, the present disclosure provides a nucleic acid or plurality of nucleic acids encoding the chimeric nuclease or the guide RNA of the present disclosure. In some embodiments, the chimeric nuclease or the guide RNA is operably coupled to a eukaryotic promoter, an enhancer, a polyadenylation site, or a combination thereof. In some embodiments, the nucleic acid is an expression vector selected from a plasmid, a lentivirus vector, an adeno associated virus vector, or an adenovirus vector. In some embodiments, the nucleic acid or plurality of nucleic acids further comprise the donor nucleic acid portion.

In another aspect, the present disclosure provides a method of targeting the oncogenic mutation in a cell comprising contacting the composition of the present disclosure to the cell. In some embodiments, the cell is a cell in an individual afflicted with cancer.

In another aspect, the present disclosure provides a use of the composition of the present disclosure to the cell for targeting the oncogenic mutation in a cell. In some embodiments, the cell is a cell in an individual afflicted with cancer

In another aspect, the present disclosure provides a method of editing a genome in a cell comprising contacting the composition of the present disclosure to the cell. In some embodiments, the cell is a cell in an individual afflicted with cancer.

In another aspect, the present disclosure provides a use of the composition of the present disclosure for editing a genome in a cell. In some embodiments, the cell is a cell in an individual afflicted with cancer.

In another aspect, the present disclosure provides a method of deleting at least a portion of the oncogenic mutation in a cell comprising contacting the composition of the present disclosure to the cell. In some embodiments, the cell is a cell in an individual afflicted with cancer.

In another aspect, the present disclosure provides a use of the composition of the present disclosure for deleting at least a portion of the oncogenic mutation in a cell. In some embodiments, the cell is a cell in an individual afflicted with cancer.

In another aspect, the present disclosure provides a method of silencing or disrupting at least a portion of the oncogenic mutation in a cell comprising contacting the composition of the present disclosure to the cell. In some embodiments, the cell is a cell in an individual afflicted with cancer.

In another aspect, the present disclosure provides a use of the composition of the present disclosure for silencing or disrupting at least a portion of the oncogenic mutation in a cell. In some embodiments, the cell is a cell in an individual afflicted with cancer.

In another aspect, the present disclosure provides a method of replacing at least a portion of the oncogenic mutation in a cell comprising contacting the composition of the present disclosure to the cell. In some embodiments, the cell is a cell in an individual afflicted with cancer.

In another aspect, the present disclosure provides a use of the composition of the present disclosure for replacing at least a portion of the oncogenic mutation in a cell. In some embodiments, the cell is a cell in an individual afflicted with cancer.

In another aspect, the present disclosure provides a method of restoring a non-oncogenic function in a cell comprising contacting the composition of the present disclosure to the cell. In some embodiments, the cell is a cell in an individual afflicted with cancer.

In another aspect, the present disclosure provides a use of the composition of the present disclosure for restoring a non-oncogenic function in a cell. In some embodiments, the cell is a cell in an individual afflicted with cancer.

In another aspect, the present disclosure provides a method of treating cancer in an individual, comprising administering the composition of the present disclosure to the individual with cancer, thereby treating the cancer in the individual.

In another aspect, the present disclosure provides a use of the composition of the present disclosure for treatment of cancer in an individual.

In another aspect, the present disclosure provides a composition, comprising: a chimeric nuclease, wherein the chimeric nuclease comprises an I-TEVI nuclease domain, an RNA-guided nuclease Cas domain, and a guide RNA, wherein the guide RNA comprises a nucleic acid sequence that targets an oncogenic mutation, wherein the oncogenic mutation is (i) an insertion of one or more nucleotides, or (ii) a substitution or deletion of 10 or less nucleotides.

In some embodiments, the oncogenic mutation is a single nucleotide polymorphism. In some embodiments, a sequence comprising the oncogenic mutation is selected from a mutation set forth in any one of SEQ ID NOs: 1-683, or a combination thereof. In some embodiments, a sequence comprising the oncogenic mutation is at least 85%, 90%, 95%, 97%, 98%, or 99% identical to a mutation set forth in any one of SEQ ID NOs: 1-683, or a combination thereof. In some embodiments, the oncogenic mutation comprises a mutation corresponding an EGFR L858R mutation or an EGFR V769_D770insASV mutation. In some embodiments, the oncogenic mutation comprises a mutation corresponding to an EGFR L858R mutation. In some embodiments, the guide RNA hybridizes to a target nucleotide sequence set forth in SEQ ID NO: 45, 130, or 141, or comprises a nucleotide sequence as set forth in SEQ ID NO: 1045, I130, 1141, or 1686. In some embodiments, the guide RNA comprises a nucleic acid sequence that hybridizes to a target nucleic acid sequence at least 85%, 90%, 95%, 97%, 98%, or 99% identical to any one of SEQ ID NO: 45, 130, 141, or comprises a nucleotide sequence at least 85%, 90%, 95%, 97%, 98%, or 99% identical to any one of SEQ ID NO: 1045, I130, I141, or 1686. In some embodiments, the oncogenic mutation comprises a mutation corresponding to an EGFR V769_D770insASV mutation. In some embodiments, the guide RNA hybridizes to a target nucleotide sequence set forth in SEQ ID NO: 683, or comprises a nucleotide sequence as set forth in SEQ ID NO: 1683 or 1684. In some embodiments, the guide RNA comprises a nucleic acid sequence that hybridizes to a target nucleic acid sequence at least 85%, 90%, 95%, 97%, 98%, or 99% identical to any one of SEQ ID NO: 683, or comprises a nucleotide sequence at least 85%, 90%, 95%, 97%, 98%, or 99% identical to any one of SEQ ID NO: 1683 or 1684. In some embodiments, the oncogenic mutation is an oncogenic mutation to a gene selected from any one of Muc4, PIK3CA, KRAS, or a combination thereof. In some embodiments, the oncogenic mutation comprises a Muc4 mutation. In some embodiments, the Muc4 mutation is an in-frame deletion of exon 2 or an in-frame deletion of exon 3. In some embodiments, the Muc4 mutation comprises a mutation corresponding to any one of positions P1542, P1680, T1711, V1721, P1826, A1830, S3560, A1833, D2253, V2281, P3088, T3119, T3183, V3817, A3902 of human Muc4 protein, or a combination thereof. In some embodiments, the Muc4 mutation is selected from a mutation corresponding to any one of P1542L, P1680S, T17111, V1721A, P1826H, A1830T, S3560S, A1833V, D2253H, V2281AM, P3088L, T3119T, T3183M, V3817A, A3902V of human Muc4 protein, or a combination thereof. In some embodiments, the guide RNA hybridizes to a target nucleotide sequence set forth in SEQ ID NO: 676, 677, 678, 679 or 682, or comprises a nucleotide sequence as set forth in SEQ ID NO: 1676, I677, I678, 1679, I682, or 1685. In some embodiments, the guide RNA comprises a nucleic acid sequence that hybridizes to a target nucleic acid sequence at least 85%, 90%, 95%, 97%, 98%, or 99% identical to any one of SEQ ID NO: 676, 677, 678, 679 or 682, or comprises a nucleotide sequence at least 85%, 90%, 95%, 97%, 98%, or 99% identical to any one of SEQ ID NO: 1676, 1677, I678, I679, I682, or 1685. In some embodiments, the oncogenic mutation comprises a PIK3CA mutation. In some embodiments, the PIK3CA mutation comprises a mutation corresponding to any one of positions H1047, E542, E545, N345, C1636, G1624, G1633, A3140, C3075, A1634, A1173 of human PIK3A protein, or a combination thereof. In some embodiments, the PIK3CA mutation is selected from a mutation corresponding to any one of H1047R, H1047L, E542K, E545K, N345K, C1636A, G1624A, G1633A, A3140T, A3140G, C3075T, A1634C, A1173G of human PIK3A protein, or a combination thereof. In some embodiments, the guide RNA hybridizes to a target nucleotide sequence set forth in SEQ ID NO: 5, 6, 7, 8, 33, 202, 204, 209 or 210, or comprises a nucleotide sequence as set forth in SEQ ID NO: 1005, 1006, 1007, 1008, 1033, 1202, 1204, 1209, or 1210. In some embodiments, the guide RNA comprises a nucleic acid sequence that hybridizes to a target nucleic acid sequence at least 85%, 90%, 95%, 97%, 98%, or 99% identical to any one of SEQ ID NO: 5, 6, 7, 8, 33, 202, 204, 209 or 210, or comprises a nucleotide sequence at least 85%, 90%, 95%, 97%, 98%, or 99% identical to any one of SEQ ID NO: 1005, 1006, 1007, 1008, 1033, 1202, 1204, 1209, or 1210. In some embodiments, the oncogenic mutation comprises a KRAS mutation. In some embodiments, the KRAS mutation comprises a mutation selected from a mutation corresponding to any one of positions A59, D119, D33, G21, G12, G13, Q61, A146, K117 of human KRAS protein, or a combination thereof. In some embodiments, the KRAS mutation is selected from a mutation corresponding to any one of A59T, A59E, A59T, D119N, D33E, G21C, G12C, G12D, G12V, G12R, G12A, G12S, G13D, G13C, G13V, G13R, Q61R, Q61V, Q61L, Q61K, Q61H, Q61A, Q61P, Q61E, A146T, A146V, K117N, K117R of human KRAS protein, or a combination thereof. In some embodiments, the guide RNA hybridizes to a target nucleotide sequence set forth in SEQ ID NO: 37, 42, 51, 52, 62, 63, or 77, or comprises a nucleotide sequence as set forth in SEQ ID NO: 1037, 1042, 1051, 1052, 1062, 1063, or 1077. In some embodiments, the guide RNA comprises a nucleic acid sequence that hybridizes to a target nucleic acid sequence at least 85%, 90%, 95%, 97%, 98%, or 99% identical to any one of SEQ ID NO: 37, 42, 51, 52, 62, 63, or 77, or comprises a nucleotide sequence at least 85%, 90%, 95%, 97%, 98%, or 99% identical to any one of SEQ ID NO: 1037, 1042, 1051, 1052, 1062, 1063, or 1077. In some embodiments, the guide RNA comprises one or more of: a non-natural internucleoside linkage, a nucleic acid mimetic, a modified sugar moiety, or a modified nucleobase. In some embodiments, the non-natural internucleoside linkage comprises one or more of: a phosphorothioate, a phosphoramidate, a non-phosphodiester, a heteroatom, a chiral phosphorothioate, a phosphorodithioate, a phosphotriester, an aminoalkylphosphotriester, a 3′-alkylene phosphonates, a 5′-alkylene phosphonate, a chiral phosphonate, a phosphinate, a 3′-amino phosphoramidate, an aminoalkylphosphoramidate, a phosphorodiamidate, a thionophosphoramidate, a thionoalkylphosphonate, a thionoalkylphosphotriester, a selenophosphate, or a boranophosphate. In some embodiments, the nucleic acid mimetic comprises one or more of a peptide nucleic acid (PNA), morpholino nucleic acid, cyclohexenyl nucleic acid (CeNAs), or a locked nucleic acid (LNA). In some embodiments, the modified sugar moiety comprises one or more of 2′-O-(2-methoxyethyl), 2′-dimethylaminooxyethoxy, 2′-dimethylaminoethoxyethoxy, 2′-O-methyl, or 2′-fluoro. In some embodiments, the modified nucleobase comprises one or more of: a 5-methylcytosine; a 5-hydroxymethyl cytosine; a xanthine; a hypoxanthine; a 2-aminoadenine; a 6-methyl derivative of adenine; a 6-methyl derivative of guanine; a 2-propyl derivative of adenine; a 2-propyl derivative of guanine; a 2-thiouracil; a 2-thiothymine; a 2-thiocytosine; a 5-halouracil; a 5-halocytosine; a 5-propynyl uracil; a 5-propynyl cytosine; a 6-azo uracil; a 6-azo cytosine; a 6-azo thymine; a pseudouracil; a 4-thiouracil; an 8-halo; an 8-amino; an 8-thiol; an 8-thioalkyl; an 8-hydroxyl; a 5-halo; a 5-bromo; a 5-trifluoromethyl; a 5-substituted uracil; a 5-substituted cytosine; a 7-methylguanine; a 7-methyladenine; a 2-Fadenine; a 2-amino-adenine; an 8-azaguanine; an 8-azaadenine; a 7-deazaguanine; a 7-deazaadenine; a 3-deazaguanine; a 3-deazaadenine; a tricyclic pyrimidine; a phenoxazine cytidine; a phenothiazine cytidine; a substituted phenoxazine cytidine; a carbazole cytidine; a pyridoindole cytidine; a 7-deaza-adenine; a 7-deazaguanosine; a 2-aminopyridine; a 2-pyridone; a 5-substituted pyrimidine; a 6-azapyrimidine; an N-2, N-6 or 0-6 substituted purine; a 2-aminopropyladenine; a 5-propynyluracil; or a 5-propynylcytosine. In some embodiments, the composition further comprises a linker that is operably linked to the I-TEVI nuclease domain and the RNA-guided nuclease Cas domain. In some embodiments, the linker comprises an amino acid sequence as set forth in SEQ ID NO: 701, 702, 703, or 704. In some embodiments, the linker comprises a mutation corresponding to any one of positions T95, S101, A119, K120, K135, P126, D127, N140, T147, Q158, A161, V117, S165, or a combination thereof. In some embodiments, the linker comprises a mutation selected from a mutation corresponding to any one of T95S, S101Y, A119D, K120N, K135N, K135R, P126S, D127K, N140S, T147I, Q158R, A161V, V117F, S165G, or a combination thereof. In some embodiments, the linker comprises an amino acid sequence that is at least 85%, 90%, 95%, 97%, 98%, or 99% identical to SEQ ID NO: 701, 702, 703, or 704. In some embodiments, the linker comprises a mutation corresponding to any one of positions T95, S101, A119, K120, K135, P126, D127, N140, T147, Q158, A161, V117, S165, or a combination thereof. In some embodiments, the linker comprises a mutation selected from a mutation corresponding to any one of T95S, S101Y, A119D, K120N, K135N, K135R, P126S, D127K, N140S, T147I, Q158R, A161V, V117F, S165G, or a combination thereof. In some embodiments, the RNA-guided nuclease Cas domain is a RNA-guided nuclease Cas9 domain. In some embodiments, the RNA-guided nuclease Cas9 domain is any one of an RNA-guided nuclease Staphylococcus aureus Cas9 domain, an RNA-guided nuclease Streptococcus pyogenes Cas9 domain, an RNA-guided nuclease Neisseria meningitidis Cas9 domain, an RNA-guided nuclease Campylobacter jejuni Cas9 domain, an RNA-guided nuclease Streptococcus pasteurianus Cas9 domain, an RNA-guided nuclease Streptococcus pasteurianus Cas9 domain, an RNA-guided nuclease Clostridium cellulolyticum Cas9 domain, an RNA-guided nuclease Geobacillus thermodenitrificans T1 Cas9 domain, or combination thereof. In some embodiments, the RNA-guided nuclease Cas9 domain is an RNA-guided nuclease Staphylococcus aureus Cas9 domain. In some embodiments, the RNA-guided nuclease Staphylococcus aureus Cas9 domain comprises an amino acid sequence as set forth in SEQ ID NO: 710. In some embodiments, the RNA-guided nuclease Staphylococcus aureus Cas9 domain comprises a mutation corresponding to any one of positions D10, H557, N580, H840, D1135, R1335, T1337, T267, L325, V327, D333, A336, 1341, E345, D348, K352, S360, T368, N369, N371, S372, E373, K386, N393, H408, N410, 1414, A415, T438, Y467, N471, D485, M489, E506, R409, T510, N515, Y518, A539, F550, N551, S596, T602, A611, 1617, T620, G654, N667, R685, K695, 1706, K722, A723, K724, M731, F732, K735, S739, P741, E742, E746, Q747, 1754, T755, H757, K760, H761, P778, E781, 1783, N784, D785, T786L, L787, Y788, K792, D794, T798, L799, V801, N803, L804, N805, G806, D813, K814, L818, 1819, S822, E824, L841, G847, D848, Y857, V875, 1876, N884, A888, L890, D894, D895, P897, V903, G920, F924, N929, E936, N937, V941, N942, 5943, C945, E947, K951, L952, S956, N957, Q958, A959, N974, G975, V983, N984, N985, D986, I991, V993, M995, I996, T999, Y1000, R1001, E1002, L1004, E1005, N1006, M1007, D1009, K1010, R1011, P1012, P1013, I1015, I1016, A1020, 51021, Q1024, K1027, E1039, H1045, 10148, K1050 or a combination thereof. In some embodiments, the RNA-guided nuclease Staphylococcus aureus Cas9 domain comprises a mutation selected from a mutation corresponding to any one of D10A, D10E, H557A, N580A, H840A, D1135E, R1335Q, T1337R, T267A, L325F, V327I, D333G, A336S, I341L, E345D, D348N, K352E, S360A, T368A, N369E, N371E, S372P, E373K, K386T, N393R, H408N, N410S, I414M, A415T, T438S, Y467F, N471K, D485E, M489F, E506K, R409K, T510E, N515K, Y518F, A539P, F550Y, N551H, S596A, T602I, A611S, I617V, T620K, G654E, N667D, R685K, K695Q, I706V, K722T, A723T, K724N, M73I T, F732V, K735Q, S739N, P741L, E742G, E746D, Q747D, I754D, T755I, H757R, K760Q, H761S, P778I, E781K, I783V, N784D, D785E, T786L, L787V, Y788H, K792E, D794T, T798R, L799I, V801I, N803S, L804I, N805K, G806N, D813G, K814E, L8181, 1819F, S822P, E824G, L841T, G847S, D848N, Y857H, V8751, 1876V, N884K, A888V, L890R, D894G, D895H, P897L, V903I, G920D, F924L, N929Y, E936D, N937G, V941I, N942D, S943L, C945A, E947K, K951R, L952Q, S956N, N957E, Q958K, A959S, N974D, G975K, V983A, N984S, N985D, D986G, I991V, V993L, M995F, I996V, T999N, Y1000K, R1001E, E1002D, L1004I, E1005K, N1006M, M1007N, D1009L, K1010S, R1011T, P1012S, P1013F, I1015L, I1016R, A1020G, 51021K, Q1024K, K1027S, E1039K, H1045K, I0148M, K1050M or a combination thereof. In some embodiments, the RNA-guided nuclease Staphylococcus aureus Cas9 domain comprises an amino acid sequence that is at least 85%, 90%, 95%, 97%, 98%, or 99% identical to SEQ ID NO: 710. In some embodiments, the RNA-guided nuclease Staphylococcus aureus Cas9 domain comprises a mutation corresponding to any one of positions D10, H557, N580, H840, D1135, R1335, T1337, T267, L325, V327, D333, A336, 1341, E345, D348, K352, S360, T368, N369, N371, S372, E373, K386, N393, H408, N410, 1414, A415, T438, Y467, N471, D485, M489, E506, R409, T510, N515, Y518, A539, F550, N551, S596, T602, A611, I617, T620, G654, N667, R685, K695, 1706, K722, A723, K724, M731, F732, K735, S739, P741, E742, E746, Q747, 1754, T755, H757, K760, H761, P778, E781, 1783, N784, D785, T786L, L787, Y788, K792, D794, T798, L799, V801, N803, L804, N805, G806, D813, K814, L818, 1819, S822, E824, L841, G847, D848, Y857, V875, 1876, N884, A888, L890, D894, D895, P897, V903, G920, F924, N929, E936, N937, V941, N942, 5943, C945, E947, K951, L952, S956, N957, Q958, A959, N974, G975, V983, N984, N985, D986, I991, V993, M995, I996, T999, Y1000, R1001, E1002, L1004, E1005, N1006, M1007, D1009, K1010, R1011, P1012, P1013, I1015, 11016, A1020, S1021, Q1024, K1027, E1039, H1045, 10148, K1050 or a combination thereof. In some embodiments, the RNA-guided nuclease Staphylococcus aureus Cas9 domain comprises a mutation selected from a mutation corresponding to any one of D10A, D10E, H557A, N580A, H840A, D1135E, R1335Q, T1337R, T267A, L325F, V327I, D333G, A336S, I341L, E345D, D348N, K352E, S360A, T368A, N369E, N371E, S372P, E373K, K386T, N393R, H408N, N410S, I414M, A415T, T438S, Y467F, N471K, D485E, M489F, E506K, R409K, T510E, N515K, Y518F, A539P, F550Y, N551H, S596A, T602I, A611S, I617V, T620K, G654E, N667D, R685K, K695Q, I706V, K722T, A723T, K724N, M73I T, F732V, K735Q, S739N, P741L, E742G, E746D, Q747D, I754D, T755I, H757R, K760Q, H761S, P778I, E781K, I783V, N784D, D785E, T786L, L787V, Y788H, K792E, D794T, T798R, L799I, V801I, N803S, L804I, N805K, G806N, D813G, K814E, L8181, 1819F, S822P, E824G, L841T, G847S, D848N, Y857H, V8751, 1876V, N884K, A888V, L890R, D894G, D895H, P897L, V903I, G920D, F924L, N929Y, E936D, N937G, V941I, N942D, S943L, C945A, E947K, K951R, L952Q, S956N, N957E, Q958K, A959S, N974D, G975K, V983A, N984S, N985D, D986G, I991V, V993L, M995F, I996V, T999N, Y1000K, R1001E, E1002D, L1004I, E1005K, N1006M, M1007N, D1009L, K1010S, R1011T, P1012S, P1013F, I1015L, I1016R, A1020G, 51021K, Q1024K, K1027S, E1039K, H1045K, I0148M, K1050M or a combination thereof. In some embodiments, the RNA-guided nuclease Staphylococcus aureus Cas9 domain comprises a mutation corresponding to the D10E mutation. In some embodiments, the RNA-guide nuclease Cas9 domain is an RNA-guided nuclease Streptococcus pyogenes Cas9 domain. In some embodiments, the RNA-guided nuclease Streptococcus pyogenes Cas9 domain comprises an amino acid sequence as set forth in SEQ ID NO: 711. In some embodiments, the RNA-guided nuclease Streptococcus pyogenes Cas9 domain comprises a mutation corresponding to any one of positions D10, S29, F32, D39, R40, H41, S42,148, C80, S87, K112, H113, K132, K141, D147, L158, E171, P176, I186, V189, Q190, Q194, N199, 1201, N202, A203, S204, R205, A210, Q228, L229, G231, S245, T249, S254, D261, T270, N295, T300, D304, V308, N309, I312, T333, A337, E345, F352, Q354, S355, K356, G366, A367, E396, L398, 1414, D428, F429, D435, K468, S469, E470, T472, E480, A486, S490, F498, K500, N501, N504, K528, V530, E532, G533, A538, T555, K570, F575, D605, E611, R629, E634, T638, R655, R664, R671, K705, E706, Q709, K710, S714, G7115, G717, H721, H723, A725, N726, V743, L747, V748, K772, K775, N776, 1788, G792, K797, Y799, T804, N808, L811, R820, N831, R832, V842, L847, N869, E874, N881, Q885, N888, T893, L911, Y945, D946, L949, E952, A1023, Y1036, G1067, G1077, R1078, N1093, R1114, N1115, D1117, A1121, D1125, P1128, K1129, V1146, S1154, S1159, L1164, S1172, N1177, P1178, I1179, D1180, K1211, M1213, G1218, N1234, E1243, K1244, E1253, E1260, K1263, H1264, E1271, Q1272, E1275, V1290, L1291, S1292, A1293, N1295, H1297, R1298, D1299, K1300, R1303, E1307, N1308, I1309, I1310, H1311, L1312, L1315, T1316, N1317, Y1326, D1328, V1342, A1345, I1360, S1363, or a combination thereof. In some embodiments, the RNA-guided nuclease Streptococcus pyogenes Cas9 domain comprises a mutation selected from a mutation corresponding to any one of D10E, D10A, S29T, F32M, D39N, R40K, H41Q, S42T, I48L, C80R, S87A, K112D, H113N, K132N, K141E, D147E, L158V, E171Q, P176S, I186K, V189L, Q190H, Q194E, N199R, I201L, N202E, A203E, S204I, R205K, A210G, Q228A, L229F, G23I N, S245A, T249M, S254A, D261N, T270S, N295K, T300I, D304G, V308A, N309D, I312V, T333A, A337V, E345K, F352S, Q354K, S355T, K356T, G366K, A367T, E396D, L398F, I414V, D428A, F429Y, D435E, K468Q, S469R, E470N, T472A, E480D, A486T, S490L, F498V, K500E, N501H, N504T, K528R, V530I, E532D, G533E, A538E, T555A, K570Q, F575C, D605E, E611D, R629K, E634K, T638K, R655H, R664K, R671K, K705V, E706D, Q709K, K710A, S714F, G7115E, G717K, H721K, H723Q, A725S, N726A, V743I, L747I, V748I, K772Q, K775R, N776R, I788M, G792R, K797E, Y799H, T804A, N808D, L811R, R820K, N83I D, R832H, V842I, L847I, N869D, E874A, N881S, Q885R, N888K, T893S, L911A, Y945H, D946G, L949P, E952A, A1023G, Y1036R, G1067E, G1077E, R1078K, N1093T, R1114G, N1115E, D1117A, A1121P, D1125G, P1128T, K1129T, V11461, S1154T, S1159P, L1164V, S1172N, N1177D, P1178S, 11179V, D1180S, K1211R, M1213L, G1218T, N1234H, E1243D, K1244T, E1253K, E1260D, K1263Q, H1264Y, E1271D, Q1272W, E1275H, V1290L, L1291R, S1292A, A1293T, N1295E, H1297N, R1298T, D1299H, K1300L, R1303S, E1307D, N1308S, I1309M, I1310L, H131I N, L1312A, L1315F, T1316S, N1317R, Y1326F, D1328N, V1342I, A1345S, I1360L, S1363N, or a combination thereof. In some embodiments, the RNA-guided nuclease Streptococcus pyogenes Cas9 domain comprises an amino acid sequence that is at least 85%, 90%, 95%, 97%, 98%, or 99% identical to SEQ ID NO: 711. In some embodiments, the RNA-guided nuclease Staphylococcus pyogenes Cas9 domain comprises a mutation corresponding to any one of positions D10, S29, F32, D39, R40, H41, S42, I48, C80, S87, K112, H113, K132, K141, D147, L158, E171, P176, I186, V189, Q190, Q194, N199, 1201, N202, A203, S204, R205, A210, Q228, L229, G231, S245, T249, S254, D261, T270, N295, T300, D304, V308, N309, I312, T333, A337, E345, F352, Q354, S355, K356, G366, A367, E396, L398, 1414, D428, F429, D435, K468, S469, E470, T472, E480, A486, S490, F498, K500, N501, N504, K528, V530, E532, G533, A538, T555, K570, F575, D605, E611, R629, E634, T638, R655, R664, R671, K705, E706, Q709, K710, S714, G7115, G717, H721, H723, A725, N726, V743, L747, V748, K772, K775, N776, 1788, G792, K797, Y799, T804, N808, L811, R820, N831, R832, V842, L847, N869, E874, N881, Q885, N888, T893, L911, Y945, D946, L949, E952, A1023, Y1036, G1067, G1077, R1078, N1093, R1114, N1115, D1117, A1121, D1125, P1128, K1129, V1146, S1154, S1159, L1164, S1172, N1177, P1178, I1179, D1180, K1211, M1213, G1218, N1234, E1243, K1244, E1253, E1260, K1263, H1264, E1271, Q1272, E1275, V1290, L1291, S1292, A1293, N1295, H1297, R1298, D1299, K1300, R1303, E1307, N1308, I1309, I1310, H1311, L1312, L1315, T1316, N1317, Y1326, D1328, V1342, A1345, I1360, S1363, or a combination thereof. In some embodiments, the RNA-guided nuclease Streptococcus pyogenes Cas9 domain comprises a mutation selected from a mutation corresponding to any one of D10E, D10A, S29T, F32M, D39N, R40K, H41Q, S42T, I48L, C80R, S87A, K112D, Hi 13N, K132N, K141E, D147E, L158V, E171Q, P176S, I186K, V189L, Q190H, Q194E, N199R, I201L, N202E, A203E, S204I, R205K, A210G, Q228A, L229F, G23I N, S245A, T249M, S254A, D261N, T270S, N295K, T300I, D304G, V308A, N309D, I312V, T333A, A337V, E345K, F352S, Q354K, S355T, K356T, G366K, A367T, E396D, L398F, I414V, D428A, F429Y, D435E, K468Q, S469R, E470N, T472A, E480D, A486T, S490L, F498V, K500E, N501H, N504T, K528R, V530I, E532D, G533E, A538E, T555A, K570Q, F575C, D605E, E61 ID, R629K, E634K, T638K, R655H, R664K, R671K, K705V, E706D, Q709K, K710A, S714F, G7115E, G717K, H721K, H723Q, A725S, N726A, V743I, L747I, V748I, K772Q, K775R, N776R, I788M, G792R, K797E, Y799H, T804A, N808D, L811R, R820K, N83ID, R832H, V842I, L847I, N869D, E874A, N881S, Q885R, N888K, T893S, L91IA, Y945H, D946G, L949P, E952A, A1023G, Y1036R, G1067E, G1077E, R1078K, N1093T, R1114G, N1115E, D1117A, A1121P, D1125G, P1128T, K1129T, V11461, 51154T, 51159P, L1164V, 51172N, N1177D, P1178S, 11179V, D1180S, K1211R, M1213L, G1218T, N1234H, E1243D, K1244T, E1253K, E1260D, K1263Q, H1264Y, E1271D, Q1272W, E1275H, V1290L, L1291R, S1292A, A1293T, N1295E, H1297N, R1298T, D1299H, K1300L, R1303S, E1307D, N1308S, I1309M, I1310L, H1311N, L1312A, L1315F, T1316S, N1317R, Y1326F, D1328N, V1342I, A1345S, 11360L, S1363N, or a combination thereof. In some embodiments, the RNA-guided nuclease Cas9 domain is an RNA-guided nuclease Neisseria meningitidis Cas9 domain. In some embodiments, the RNA-guided nuclease Neisseria meningitidis Cas9 domain comprises an amino acid sequence as set forth in SEQ ID NO: 712. In some embodiments, the RNA-guided nuclease Neisseria meningitidis Cas9 domain comprises a mutation corresponding to any one of positions I9, D16, D30, E31, A94, I103, P124, N164, I213, G229, T241, 5376, E393, G454, K471, G490, D660, C665, K764, T770, P803, A841, H842, K843, D844, L846, R847, K854, H855, N856, K858, K862, W865, E868, 1869, A872, D873, N876, Y880, G883, 1886, E887, E890, R895, A898, Y899, G900, G901, N902, A903, K904, Q905, D908, N912, K917, G919, L921, V927, K929, T930, E932, S933, L936, L937, N938, K939, K940, Y943, T944, G949, D950, C958, K965, N966, Q967, F969, A975, E980, N981, I986, D987, C988, K989, G990, Y991, R992, I993, D994, Y997, T998, C1000, S1002, H1004, K1005, Y1006, A1010, F1011, Q1012, K1013, D1014, E1015, K1018, V1019, E1020, F1021, A1022, Y1024, I1025, N1026, C1027, D1028, S1029, S1030, N1031, R1033, F1034, Y1035, L1036, A1037, W1038, K1041, G1042, K1044, E1045, Q1046, Q1047, F1048, R1049, I1050, S1051, T1052, Q1053, N1054, L1055, V1056, L1057, I1058, Y1061, V1063, N1064, or a combination thereof. In some embodiments, the RNA-guided nuclease Neisseria meningitidis Cas9 domain comprises a mutation selected from a mutation corresponding to any one of I9M, D16E, D30E, E31K, A94D, I103V, P124C, N164D, I213N, G229D, T241A, S376T, E393K, G454C, K471E, G490C, D660E, C665R, K764E, T770A, P803S, A841Q, H842G, K843H, D844E, L846V, R847K, K854R, H855L, N856D, K858G, K862L, W865P, E868Q, I869L, A872K, D873G, N876K, Y880R, G883E, I886P, E887K, E890E, R895Q, A898T, Y899H, G900K, G901D, N902D, A903P, K904T, Q905K, D908A, N912E, K917Y, G919T, L921Q, V927I, K929Q, T930V, E932K, S933T, L936W, L937V, N938R, K939N, K940H, Y943N, T944G, G949A, D950T, C958E, K965G, N966G, Q967K, F969Y, A975S, E980K, N981G, I986R, D987A, C988V, K989V, G990A, Y991F, R992K, I993D, D994E, Y997F, T998E, C1000R, S1002I, H1004Y, K1005A, Y1006N, A1010K, F1011L, Q1012T, K1013A, D1014K, E1015K, K1018N, V1019E, E1020F, F1021L, A1022G, Y1024F, I1025V, N1026S, C1027L, D1028N, S1029R, S1030A, N103IT, R1033A, F1034I, Y1035D, L1036I, A1037R, W1038T, K1041T, G1042D, K1044T, E1045K, Q1046G, Q1047E, F1048Q, R1049S, I1050V, S1051G, T1052V, Q1053K, N1054T, L1055A, V1056L, L1057S, I1058F, Y1061N, V1063I, N1064D, or a combination thereof. In some embodiments, the RNA-guided nuclease Neisseria meningitidis Cas9 domain comprises an amino acid sequence that is at least 85%, 90%, 95%, 97%, 98%, or 99% identical to SEQ ID NO: 712. In some embodiments, the RNA-guided nuclease Neisseria meningitidis Cas9 domain comprises a mutation corresponding to any one of positions 19, D16, D30, E31, A94, I103, P124, N164, I213, G229, T241, S376, E393, G454, K471, G490, D660, C665, K764, T770, P803, A841, H842, K843, D844, L846, R847, K854, H855, N856, K858, K862, W865, E868, 1869, A872, D873, N876, Y880, G883, 1886, E887, E890, R895, A898, Y899, G900, G901, N902, A903, K904, Q905, D908, N912, K917, G919, L921, V927, K929, T930, E932, S933, L936, L937, N938, K939, K940, Y943, T944, G949, D950, C958, K965, N966, Q967, F969, A975, E980, N981, I986, D987, C988, K989, G990, Y991, R992, I993, D994, Y997, T998, C1000, S1002, H1004, K1005, Y1006, A1010, F1011, Q1012, K1013, D1014, E1015, K1018, V1019, E1020, F1021, A1022, Y1024, I1025, N1026, C1027, D1028, S1029, S1030, N1031, R1033, F1034, Y1035, L1036, A1037, W1038, K1041, G1042, K1044, E1045, Q1046, Q1047, F1048, R1049, I1050, S1051, T1052, Q1053, N1054, L1055, V1056, L1057, I1058, Y1061, V1063, N1064, or a combination thereof. In some embodiments, the RNA-guided nuclease Neisseria meningitidis Cas9 domain comprises a mutation selected from a mutation corresponding to any one of I9M, D16E, D30E, E31K, A94D, I103V, P124C, N164D, I213N, G229D, T241A, S376T, E393K, G454C, K471E, G490C, D660E, C665R, K764E, T770A, P803S, A841Q, H842G, K843H, D844E, L846V, R847K, K854R, H855L, N856D, K858G, K862L, W865P, E868Q, I869L, A872K, D873G, N876K, Y880R, G883E, I886P, E887K, E890E, R895Q, A898T, Y899H, G900K, G901D, N902D, A903P, K904T, Q905K, D908A, N912E, K917Y, G919T, L921Q, V927I, K929Q, T930V, E932K, S933T, L936W, L937V, N938R, K939N, K940H, Y943N, T944G, G949A, D950T, C958E, K965G, N966G, Q967K, F969Y, A975S, E980K, N981G, I986R, D987A, C988V, K989V, G990A, Y991F, R992K, I993D, D994E, Y997F, T998E, C1000R, S1002I, H1004Y, K1005A, Y1006N, A1010K, F1011L, Q1012T, K1013A, D1014K, E1015K, K1018N, V1019E, E1020F, F1021L, A1022G, Y1024F, I1025V, N1026S, C1027L, D1028N, S1029R, S1030A, N1031T, R1033A, F1034I, Y1035D, L1036I, A1037R, W1038T, K1041T, G1042D, K1044T, E1045K, Q1046G, Q1047E, F1048Q, R1049S, I1050V, S1051G, T1052V, Q1053K, N1054T, L1055A, V1056L, L1057S, I1058F, Y1061N, V1063I, N1064D, or a combination thereof. In some embodiments, the RNA-guide nuclease Cas9 domain is an RNA-guided nuclease Campylobacter jejuni Cas9 domain. In some embodiments, the RNA-guided nuclease Campylobacterjejuni Cas9 domain comprises an amino acid sequence as set forth in SEQ ID NO: 713. In some embodiments, the RNA-guided nuclease Campylobacter jejuni Cas9 domain comprises a mutation corresponding to any one of positions L5, A6, D8, I9, S12, S13, F18, S19, L24, K25, 131, T40, E42, L50, L58, A59, R61, L58, L65, H67AN74, K77, L98, I99, P101, N110, L113, A119, A126, R128, I134, K140, A144, K147, Q151, L156, V184, S190, F199, D202, G203, R212, F214, K221, E223, Y232, A235, V243, 5247, D251, P256, L261, T269, N276, N277, L285, T287, L291, K300, T305, Q308, L312, G314, Y335, K336, I339, H345, D351, N353, E354, 1362, K370, D383E, S384, K391, 1396, L403, T405, K413, N419, L421, D430, K432, A437, L453, K457, V462, A465, K472, N477, A492, E495, L525, K526, L527, K531, E532, E542, Q550, E556, H559, Y561, 5564, M572, V577, Q581, N587, N596, K600, Q602, K603, Q616, K617, N623, Y624, K633, D634, Y642, N649, D656, L660, D662, K667, V677, E680, K682, L686, H692, T693, V712, I714, V722, K723, 5736, L739, K742, L747, N751, F756, R763, Q764, E772, K777, A786, E790, F792, Q800, S801, G804, L812, E813, V833, 1835, T841, Y845, A855, L856, A863, V864, D879, E883, D900, Q902, K927, F928, V971, T972, or a combination thereof. In some embodiments, the RNA-guided nuclease Campylobacter jejuni Cas9 domain comprises a mutation selected from a mutation corresponding to any one of L5I, A6G, D8N, D8E, I9L, S12A, S13N, F18L, S19R, L24I, K25I, 131V, T40N, E42N, L50E, L58V, A59K, R61K, L58V, L65M, H67A, N74K, K77N, L98T, I99Q, P101I, N110S, L113I, A119S, A126V, R128H, I134S, K140N, A144T, K147E, Q151K, L156M, V184I, S190D, F199L, D202Q, G203E, R212K, F214L, K221K, E223K, Y232F, A235P, V243I, S247I, D251N, P256A, L261S, T269G, N276K, N277S, L285V, T287E, L291I, K300D, T305S, Q308K, L312I, G314N, Y335L, K336N, I339K, H345T, D351I, N353D, E354S, I362T, K370E, D383E, S384K, K391N, I396L, L403Q, T405I, K413R, N419E, L421C, D430E, K432S, A437L, L453I, K457C, V462L, A465D, K472S, N477H, A492K, E495I, L525Q, K526I, L527V, K531E, E532D, E542L, Q550D, E556V, H559Y, Y561R, S564N, M572S, V577T, Q581L, N587G, N596E, K600L, Q602A, K603E, Q616R, K617F, N623F, Y624F, K633T, D634E, Y642W, N649S, D656S, L660I, D662E, K667A, V677Q, E680V, K682S, L686I, H692N, T693F, V7121, I714V, V722I, K723F, S736K, L739F, K742N, L747S, N751L, F756L, R763K, Q764E, E772N, K777H, A786T, E790L, F792P, Q800N, S801T, G804D, L812V, E813K, V833S, I835L, T841K, Y845H, A855S, L856T, A863T, V864P, D879N, E883N, D900G, Q902K, K927N, F928Y, V971L, T972S, or a combination thereof. In some embodiments, the RNA-guided nuclease Campylobacter jejuni Cas9 domain comprises an amino acid sequence that is at least 85%, 90%, 95%, 97%, 98%, or 99% identical to SEQ ID NO: 713. In some embodiments, the RNA-guided nuclease Campylobacter jejuni Cas9 domain comprises a mutation corresponding to any one of positions L5, A6, D8, I9, S12, S13, F18, S19, L24, K25, 131, T40, E42, L50, L58, A59, R61, L58, L65, H67A N74, K77, L98, I99, P101, N110, L113, A119, A126, R128, I134, K140, A144, K147, Q151, L156, V184, S190, F199, D202, G203, R212, F214, K221, E223, Y232, A235, V243, 5247, D251, P256, L261, T269, N276, N277, L285, T287, L291, K300, T305, Q308, L312, G314, Y335, K336, 1339, H345, D351, N353, E354, 1362, K370, D383E, 5384, K391, 1396, L403, T405, K413, N419, L421, D430, K432, A437, L453, K457, V462, A465, K472, N477, A492, E495, L525, K526, L527, K531, E532, E542, Q550, E556, H559, Y561, 5564, M572, V577, Q581, N587, N596, K600, Q602, K603, Q616, K617, N623, Y624, K633, D634, Y642, N649, D656, L660, D662, K667, V677, E680, K682, L686, H692, T693, V712, I714, V722, K723, 5736, L739, K742, L747, N751, F756, R763, Q764, E772, K777, A786, E790, F792, Q800, S801, G804, L812, E813, V833, 1835, T841, Y845, A855, L856, A863, V864, D879, E883, D900, Q902, K927, F928, V971, T972, or a combination thereof. In some embodiments, the RNA-guided nuclease Campylobacter jejuni Cas9 domain comprises a mutation selected from a mutation corresponding to any one of L5I, A6G, D8N, D8E, I9L, S12A, S13N, F18L, S19R, L24I, K25I, 131V, T40N, E42N, L50E, L58V, A59K, R61K, L58V, L65M, H67A, N74K, K77N, L98T, I99Q, P101I, N110S, L113I, A119S, A126V, R128H, I134S, K140N, A144T, K147E, Q151K, L156M, V184I, S190D, F199L, D202Q, G203E, R212K, F214L, K221K, E223K, Y232F, A235P, V243I, S247I, D251N, P256A, L261S, T269G, N276K, N277S, L285V, T287E, L291I, K300D, T305S, Q308K, L312I, G314N, Y335L, K336N, I339K, H345T, D351I, N353D, E354S, I362T, K370E, D383E, S384K, K391N, I396L, L403Q, T405I, K413R, N419E, L421C, D430E, K432S, A437L, L453I, K457C, V462L, A465D, K472S, N477H, A492K, E495I, L525Q, K526I, L527V, K531E, E532D, E542L, Q550D, E556V, H559Y, Y561R, S564N, M572S, V577T, Q581L, N587G, N596E, K600L, Q602A, K603E, Q616R, K617F, N623F, Y624F, K633T, D634E, Y642W, N649S, D656S, L660I, D662E, K667A, V677Q, E680V, K682S, L686I, H692N, T693F, V7121, I714V, V722I, K723F, S736K, L739F, K742N, L747S, N751L, F756L, R763K, Q764E, E772N, K777H, A786T, E790L, F792P, Q800N, S801T, G804D, L812V, E813K, V833S, I835L, T841K, Y845H, A855S, L856T, A863T, V864P, D879N, E883N, D900G, Q902K, K927N, F928Y, V971L, T972S, or a combination thereof. In some embodiments, the RNA-guide nuclease Cas9 domain is an RNA-guided nuclease Streptococcus pasteurianus Cas9 domain. In some embodiments, the RNA-guided nuclease Streptococcus pasteurianus Cas9 domain comprises an amino acid sequence as set forth in SEQ ID NO: 714. In some embodiments, the RNA-guided nuclease Streptococcus pasteurianus Cas9 domain comprises a mutation corresponding to any one of positions D11, E85, A88, T92, E96, Y100, T109, D110, D113, E115, R116, D125, I127, K128, E132, S147, I185, A187, K228, Y229, T232, M255, S271, N273, A294, A327, E355, K357, N379, T380, S382, A385, D439, R440, S464, H469, Y519, I528, N569, I581, A607, K632, D633, H635, E636, A647, D648, T703, P705, K712, S713, A724, V750, D882, S951, D977, E979, S1014, H1027, I1030, E1081, D1082, D1086, K1088, S1089, N1090, R1092, T1093, I1094, C1095, A1138, Y1139, D1141, T1142, F1158, A1168, E1190, E1198, H1202, I1204, R1205, I1210, K1224, S1232, M1240, V1241, I1242, P1243, G1424, K1248, Q1254, N1257, S1258, T1262, K1263, Y1264, D1266, A1270, K1277, D1284, L1288, V1302, N1316, T1346, I1374, or a combination thereof. In some embodiments, the RNA-guided nuclease Streptococcus pasteurianus Cas9 domain comprises a mutation selected from a mutation corresponding to any one of D11E, D11A, E85D, A88T, T92A, E96D, Y100Q, T109D, D110N, D113N, E115D, R116S, D125E, I127D, K128A, E132K, S147T, I185L, A187T, K228N, Y229N, T232K, M255T, S271T, N273E, A294S, A327V, E355K, K357Q, N379G, T380I, S382T, A385N, D439E, R440E, S464A, H469R, Y519F, I528V, N569D, I581V, A607S, K632R, D633E, H635Q, E636Q, A647K, D648Q, T703A, P705S, K712E, S713A, A724T, V750I, D882G, S951R, D977E, E979K, S1014P, H1027R, I1030V, E1081G, D1082E, D1086N, K1088R, S1089T, N1090D, R1092E, T1093K, I1094V, C1095R, A1138V, Y1139L, D1141E, T1142P, F1158L, A1168T, E1190K, E1198K, H1202Q, I1204V, R1205Q, I1210M, K1224R, S1232T, M1240I, V1241M, I1242L, P1243S, G1424A, K1248A, Q1254H, N1257G, S1258N, T1262A, K1263E, Y1264H, D1266K, A1270E, K1277E, D1284N, L1288V, V1302A, N1316D, T1346N, I1374L, or a combination thereof. In some embodiments, the RNA-guided nuclease Streptococcus pasteurianus Cas9 domain comprises an amino acid sequence that is at least 85%, 90%, 95%, 97%, 98%, or 99% identical to SEQ ID NO: 714. In some embodiments, the RNA-guided nuclease Streptococcus pasteurianus Cas9 domain comprises a mutation corresponding to any one of positions D11, E85, A88, T92, E96, Y100, T109, D110, D113, E115, R116, D125, I127, K128, E132, S147, I185, A187, K228, Y229, T232, M255, S271, N273, A294, A327, E355, K357, N379, T380, S382, A385, D439, R440, S464, H469, Y519, I528, N569, I581, A607, K632, D633, H635, E636, A647, D648, T703, P705, K712, S713, A724, V750, D882, S951, D977, E979, S1014, H1027, I1030, E1081, D1082, D1086, K1088, S1089, N1090, R1092, T1093, I1094, C1095, A1138, Y1139, D1141, T1142, F1158, A1168, E1190, E1198, H1202, I1204, R1205, I1210, K1224, S1232, M1240, V1241, I1242, P1243, G1424, K1248, Q1254, N1257, S1258, T1262, K1263, Y1264, D1266, A1270, K1277, D1284, L1288, V1302, N1316, T1346, I1374, or a combination thereof. In some embodiments, the RNA-guided nuclease Streptococcus pasteurianus Cas9 domain comprises a mutation selected from a mutation corresponding to any one of D11E, D11A, E85D, A88T, T92A, E96D, Y100Q, T109D, D110N, D113N, E115D, R116S, D125E, I127D, K128A, E132K, S147T, I185L, A187T, K228N, Y229N, T232K, M255T, S271T, N273E, A294S, A327V, E355K, K357Q, N379G, T380I, S382T, A385N, D439E, R440E, S464A, H469R, Y519F, I528V, N569D, I581V, A607S, K632R, D633E, H635Q, E636Q, A647K, D648Q, T703A, P705S, K712E, S713A, A724T, V750I, D882G, S951R, D977E, E979K, S1014P, H1027R, I1030V, E1081G, D1082E, D1086N, K1088R, S1089T, N1090D, R1092E, T1093K, I1094V, C1095R, A1138V, Y1139L, D1141E, T1142P, F1158L, A1168T, E1190K, E1198K, H1202Q, I1204V, R1205Q, I1210M, K1224R, S1232T, M1240I, V1241M, I1242L, P1243S, G1424A, K1248A, Q1254H, N1257G, S1258N, T1262A, K1263E, Y1264H, D1266K, A1270E, K1277E, D1284N, L1288V, V1302A, N1316D, T1346N, I1374L, or a combination thereof. In some embodiments, the RNA-guide nuclease Cas9 domain is an RNA-guided nuclease Clostridium cellulolyticum Cas9 domain. In some embodiments, the RNA-guided nuclease Clostridium cellulolyticum Cas9 domain comprises an amino acid sequence as set forth in SEQ ID NO: 715. In some embodiments, the RNA-guided nuclease Clostridium cellulolyticum Cas9 domain comprises a mutation corresponding to any one of positions T4, D10, V9, D20, K21, 127, C33, K36, A47, A49, S64, Q65, E102, L103, T122, I1124, K131, D137, R163, G166, I1169, F170, V183, D184, I187, E193, K200, K208, L209, D221, N224, E227, F228, S234, V242, K244, L252, T256, C258, S261, V413, M415, K416, R417, K424, Y426, K427, S429, D430, A468, T470, A472, A478, Q481, K482, L485, A497, L535, W540, R541, E544, G554, P556, I1570, Y574, M580, Y584, M585, T592, D593, V606, W607, I647, N650, S693, L697, E702, S704, A713, V714, I1715, D776, L847, G850, G853, A854, R860, I900, H904, M905, I906, E921, Q923, S929, T930, H931, Q939, N994, I997, N1000, K1001, S1002, I1003, K1005, P1008, or a combination thereof. In some embodiments, the RNA-guided nuclease Clostridium cellulolyticum Cas9 domain comprises a mutation selected from any one of T4S, D10E, V9I, D20N, K21E, 127E, C33I, K36V, A47S, A49P, S64R, Q65H, E102L, L103V, T122V, I124F, K131Q, D137E, R163Q, G166S, I169L, F170L, V183G, D184G, I187T, E193S, K200Q, K208A, L209Y, D221K, N224Q, E227S, F228S, S234T, V242I, K244N, L252K, T256K, C258T, S261F, V413K, M415L, K416R, R417N, K424Q, Y426I, K427P, S429H, D430Q, A468S, T470S, A472V, A478G, Q481K, K482R, L485S, A497M, L535H, W540Y, R541K, E544Q, G554F, P556S, I570V, Y574I, M580F, Y584N, M585N, T592A, D593A, V606W, W607F, I647R, N650H, S693K, L697F, E702Q, S704N, A713V, V7141, I1715V, D776E, L847A, G850P, G853A, A854P, R860K, I900V, H904D, M905V, I906L, E921Y, Q923E, S929D, T930E, H931Y, Q939P, N994Q, I997P, N1000R, K1001M, S1002N, I1003K, K1005H, P1008K or a combination thereof. In some embodiments, the RNA-guided nuclease Clostridium cellulolyticum Cas9 domain comprises an amino acid sequence that is at least 85%, 90%, 95%, 97%, 98%, or 99% identical to SEQ ID NO: 715. In some embodiments, the RNA-guided nuclease Clostridium cellulolyticum Cas9 domain comprises a mutation corresponding to any one of positions T4, D10, V9, D20, K21, 127, C33, K36, A47, A49, S64, Q65, E102, L103, T122, I124, K131, D137, R163, G166, I1169, F170, V183, D184, I187, E193, K200, K208, L209, D221, N224, E227, F228, 5234, V242, K244, L252, T256, C258, S261, V413, M415, K416, R417, K424, Y426, K427, 5429, D430, A468, T470, A472, A478, Q481, K482, L485, A497, L535, W540, R541, E544, G554, P556, I1570, Y574, M580, Y584, M585, T592, D593, V606, W607, I647, N650, S693, L697, E702, S704, A713, V714, I1715, D776, L847, G850, G853, A854, R860, I900, H904, M905, I906, E921, Q923, S929, T930, H931, Q939, N994, I997, N1000, K1001, S1002, 11003, K1005, P1008, or a combination thereof. In some embodiments, the RNA-guided nuclease Clostridium cellulolyticum Cas9 domain comprises a mutation selected from a mutation corresponding to any one of T4S, D10E, V9I, D20N, K21E, 127E, C33I, K36V, A47S, A49P, S64R, Q65H, E102L, L103V, T122V, I124F, K131Q, D137E, R163Q, G166S, I169L, F170L, V183G, D184G, I187T, E193S, K200Q, K208A, L209Y, D221K, N224Q, E227S, F228S, S234T, V242I, K244N, L252K, T256K, C258T, S261F, V413K, M415L, K416R, R417N, K424Q, Y426I, K427P, S429H, D430Q, A468S, T470S, A472V, A478G, Q481K, K482R, L485S, A497M, L535H, W540Y, R541K, E544Q, G554F, P556S, I570V, Y574I, M580F, Y584N, M585N, T592A, D593A, V606W, W607F, I647R, N650H, S693K, L697F, E702Q, S704N, A713V, V7141, I1715V, D776E, L847A, G850P, G853A, A854P, R860K, I900V, H904D, M905V, I906L, E921Y, Q923E, S929D, T930E, H931Y, Q939P, N994Q, I997P, N1000R, K1001M, S1002N, I1003K, K1005H, P1008K or a combination thereof. In some embodiments, the RNA-guide nuclease Cas9 domain is an RNA-guided nuclease Geobacillus thermodenitrificans T1 Cas9 domain. In some embodiments, the RNA-guided nuclease Geobacillus thermodenitrificans T1 Cas9 domain comprises an amino acid sequence as set forth in SEQ ID NO: 716. In some embodiments, the RNA-guided nuclease Geobacillus thermodenitrificans T1 Cas9 domain comprises a mutation corresponding to any one of positions K2, D8, I14, D35, K41, F74, V75, K91, I117, R128, T136, Q151, S152, S156, A161, V164, S171, E178, D179, V185, R192, K195, A199, Y204, 1207, V208, A212, H215, S219, F227, T260, V261, V271, G274, I276, A278, L279, D282, I287, K289, H293, F299, V302, N307, R313, L317, L318, V331, G337, K341, 5348, A354, A355, K356, R359, M372, T377, R380, E395, D399, E404, S416, T441, R445, N464, E504, S508, M515, Q516, E520, G521, V534, L545, K559, T578, K603, T612, L619, S621, N656, N660, L673, D685, I699, N708, N717, R737, V738, 5752, D756, Q771, N777, N792, E793, 1811, 1824, K839, Q845, K848, T849, L895, I902, T908, V929, I943, I946, M948, F990, T995, V1000, Q1014, D1017, S1019, N1020, G1021, S1024, N1030, N1031, R1035, S1036, I1037, V1067, S1071, A1075, 11079, or a combination thereof. In some embodiments, the RNA-guided nuclease Geobacillus thermodenitrificans T1 Cas9 domain comprises a mutation selected from a mutation corresponding to any one of K2R, D8E, D8A, 114V, D35E, K41Q, F74V, V75I, K91E, I117V, R128K, T136S, Q151R, S152A, S156G, A161G, V164I, S171A, E178G, D179E, V185I, R192H, K195R, A199S, Y204F, I207M, V208S, A212K, H215N, S219T, F227V, T260I, V261A, V271I, G274S, I276A, A278G, L279P, D282E, I287L, K289E, H293Q, F299Y, V302I, N307R, R313Y, L317I, L318V, V331I, G337D, K341Q, S348K, A354K, A355S, K356S, R359L, M372L, T377A, R380H, E395P, D399N, E404N, S416T, T441S, R445K, N464T, E504D, S508T, M515T, Q516K, E520D, G521E, V534M, L545H, K559R, T578V, K603R, T612I, L619V, S621T, N656M, N660S, L673F, D685E, I699V, N708E, N717D, R737K, V738I, S752A, D756E, Q771R, N777H, N792D, E793Q, 1811V, I824V, K839T, Q845K, K848A, T849S, L895P, I902V, T908K, V929V, I943V, I946M, M948I, F990L, T995I, V1000G, Q1014K, D1017H, S1019G, N1020T, G1021A, S1024E, N1030C, N1031S, R1035S, S1036G, I1037V, V1067L, S1071A, A1075T, I1079V, or a combination thereof. In some embodiments, the RNA-guided nuclease Geobacillus thermodenitrificans T1 Cas9 domain comprises an amino acid sequence that is at least 85%, 90%, 95%, 97%, 98%, or 99% identical to SEQ ID NO: 716. In some embodiments, the RNA-guided nuclease Geobacillus thermodenitrificans T1 Cas9 domain comprises a mutation corresponding to any one of positions K2, D8, I14, D35, K41, F74, V75, K91, I117, R128, T136, Q151, S152, S156, A161, V164, S171, E178, D179, V185, R192, K195, A199, Y204, 1207, V208, A212, H215, S219, F227, T260, V261, V271, G274, 1276, A278, L279, D282, 1287, K289, H293, F299, V302, N307, R313, L317, L318, V331, G337, K341, S348, A354, A355, K356, R359, M372, T377, R380, E395, D399, E404, S416, T441, R445, N464, E504, S508, M515, Q516, E520, G521, V534, L545, K559, T578, K603, T612, L619, S621, N656, N660, L673, D685, I699, N708, N717, R737, V738, S752, D756, Q771, N777, N792, E793, 1811, 1824, K839, Q845, K848, T849, L895, I902, T908, V929, I943, I946, M948, F990, T995, V1000, Q1014, D1017, S1019, N1020, G1021, S1024, N1030, N1031, R1035, S1036, I1037, V1067, S1071, A1075, I1079, or a combination thereof. In some embodiments, the RNA-guided nuclease Geobacillus thermodenitrificans T1 Cas9 domain comprises a mutation selected from a mutation corresponding to any one of K2R, D8E, D8A, I14V, D35E, K41Q, F74V, V75I, K91E, I117V, R128K, T136S, Q151R, S152A, S156G, A161G, V164I, S171A, E178G, D179E, V185I, R192H, K195R, A199S, Y204F, I207M, V208S, A212K, H215N, S219T, F227V, T260I, V261A, V271I, G274S, I276A, A278G, L279P, D282E, I287L, K289E, H293Q, F299Y, V302I, N307R, R313Y, L3171, L318V, V331I, G337D, K341Q, S348K, A354K, A355S, K356S, R359L, M372L, T377A, R380H, E395P, D399N, E404N, S416T, T441S, R445K, N464T, E504D, S508T, M515T, Q516K, E520D, G521E, V534M, L545H, K559R, T578V, K603R, T612I, L619V, S621T, N656M, N660S, L673F, D685E, I699V, N708E, N717D, R737K, V738I, S752A, D756E, Q771R, N777H, N792D, E793Q, 181 IV, I824V, K839T, Q845K, K848A, T849S, L895P, I902V, T908K, V929V, I943V, I946M, M948I, F990L, T995I, V1000G, Q1014K, D1017H, S1019G, N1020T, G1021A, S1024E, N1030C, N1031S, R1035S, S1036G, I1037V, V1067L, S1071A, A1075T, 11079V, or a combination thereof. In some embodiments, the RNA-guided nuclease Cas domain is a RNA-guided nuclease Cas12 domain. In some embodiments, the RNA-guided nuclease Cas domain is a RNA-guided nuclease CasX domain. In some embodiments, the I-TEVI nuclease domain comprises an amino acid sequence that is at least 85%, 90%, 95%, 97%, 98%, or 99% identical to SEQ ID NO: 700. In some embodiments, the I-TEVI nuclease domain comprises a mutation at any one of positions corresponding to T11, V16, N14, E25, K26, R27, E36, K37, G38, C39, S41, L45, F49, I60, and E81, or a combination thereof. In some embodiments, the I-TEVI nuclease domain comprises a mutation selected from any one of corresponding to T11V, V16I, N14G, E25D, K26R, R27A, E36S, K37N, G38N, C39V, S41H, L45F, F49Y, I60V, E81I, or a combination thereof. In some embodiments, the I-TEVI nuclease domain comprises a mutation corresponding to a K26R mutation. In some embodiments, the I-TEVI nuclease domain comprises an amino acid sequence as set forth in SEQ ID NO: 700. In some embodiments, the I-TEVI nuclease domain comprises a mutation corresponding to any one of positions T11, V16, N14, E25, K26, R27, E36, K37, G38, C39, 541, L45, F49, I60, and E81, or a combination thereof. In some embodiments, the I-TEVI nuclease domain comprises a mutation selected from a mutation corresponding to any one of TiiV, V16I, N14G, E25D, K26R, R27A, E36S, K37N, G38N, C39V, S41H, L45F, F49Y, I60V, E811, or a combination thereof. In some embodiments, the I-TEVI nuclease domain comprises a mutation corresponding to a K26R mutation. In some embodiments, the chimeric nuclease further comprises a nuclear localization signal. In some embodiments, the nuclear localization signal comprises an SV40 nuclear localization signal. In some embodiments, the nuclear localization signal comprises a Nucleoplasmin nuclear localization signal. In some embodiments, the composition further comprises a donor nucleic acid. In some embodiments, the donor nucleic acid restores a non-oncogenic function of a gene comprising the oncogenic mutation. In some embodiments, the donor nucleic acid comprises a non-oncogenic version of the oncogenic mutation. In some embodiments, the donor nucleic acid is DNA. In some embodiments, the donor nucleic acid comprises a blunt end and at least two nucleotide 3′ overhang end. In some embodiments, the donor nucleic acid comprises a 5′ and a 3′ homology flanking the non-oncogenic version of the oncogenic mutation. In some embodiments, the composition does not comprise a donor nucleic acid. In some embodiments, the composition further comprises a pharmaceutically acceptable excipient, diluent or carrier. In some embodiments, the composition is encapsulated in a lipid nanoparticle. In some embodiments, the lipid nanoparticle comprises cationic or neutral lipids.

In another aspect, the present disclosure provides a nucleic acid or plurality of nucleic acids encoding the chimeric nuclease or the guide RNA of the present disclosure. In some embodiments, the chimeric nuclease or the guide RNA is operably coupled to a eukaryotic promoter, an enhancer, a polyadenylation site, or a combination thereof. In some embodiments, the nucleic acid is an expression vector selected from a plasmid, a lentivirus vector, an adeno associated virus vector, or an adenovirus vector. In some embodiments, the nucleic acid or plurality of nucleic acids further comprise the donor nucleic acid portion.

In another aspect, the present disclosure provides a method of targeting the oncogenic mutation in a cell comprising contacting the composition of the present disclosure to the cell. In some embodiments, the cell is a cell in an individual afflicted with cancer.

In another aspect, the present disclosure provides a use of the composition of the present disclosure to the cell for targeting the oncogenic mutation in a cell. In some embodiments, the cell is a cell in an individual afflicted with cancer

In another aspect, the present disclosure provides a method of editing a genome in a cell comprising contacting the composition of the present disclosure to the cell. In some embodiments, the cell is a cell in an individual afflicted with cancer.

In another aspect, the present disclosure provides a use of the composition of the present disclosure for editing a genome in a cell. In some embodiments, the cell is a cell in an individual afflicted with cancer.

In another aspect, the present disclosure provides a method of deleting at least a portion of the oncogenic mutation in a cell comprising contacting the composition of the present disclosure to the cell. In some embodiments, the cell is a cell in an individual afflicted with cancer.

In another aspect, the present disclosure provides a use of the composition of the present disclosure for deleting at least a portion of the oncogenic mutation in a cell. In some embodiments, the cell is a cell in an individual afflicted with cancer.

In another aspect, the present disclosure provides a method of silencing or disrupting at least a portion of the oncogenic mutation in a cell comprising contacting the composition of the present disclosure to the cell. In some embodiments, the cell is a cell in an individual afflicted with cancer.

In another aspect, the present disclosure provides a use of the composition of the present disclosure for silencing or disrupting at least a portion of the oncogenic mutation in a cell. In some embodiments, the cell is a cell in an individual afflicted with cancer.

In another aspect, the present disclosure provides a method of replacing at least a portion of the oncogenic mutation in a cell comprising contacting the composition of the present disclosure to the cell. In some embodiments, the cell is a cell in an individual afflicted with cancer.

In another aspect, the present disclosure provides a use of the composition of the present disclosure for replacing at least a portion of the oncogenic mutation in a cell. In some embodiments, the cell is a cell in an individual afflicted with cancer.

In another aspect, the present disclosure provides a method of restoring a non-oncogenic function in a cell comprising contacting the composition of the present disclosure to the cell. In some embodiments, the cell is a cell in an individual afflicted with cancer.

In another aspect, the present disclosure provides a use of the composition of the present disclosure for restoring a non-oncogenic function in a cell. In some embodiments, the cell is a cell in an individual afflicted with cancer.

In another aspect, the present disclosure provides a method of treating cancer in an individual, comprising administering the composition of the present disclosure to the individual with cancer, thereby treating the cancer in the individual.

In another aspect, the present disclosure provides a use of the composition of the present disclosure for treatment of cancer in an individual.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and/or advantages of the present disclosure will be obtained by reference to the following detailed description that sets forth illustrative embodiments, in which the principles of the disclosure are utilized, and the accompanying drawings (also “Figure” and “Fig.” herein), of which:

FIG. 1A is a schematic representation of the I-TevI domain (1), linker domain (2), and the Cas domain (3) within the chimeric nuclease structure.

FIG. 1B is a flow chart graphically depicting the steps undertaken by existing gene editors to produce a single cleavage that would be repaired through the error-prone non-homologous end-joining (NHEJ) pathway which may generate random indels.

FIG. 1C is a flow chart graphically depicting the steps as to how the chimeric nuclease cuts DNA at two sites using its I-TevI and Cas domains generating defined-length deletions in cells. Defined-length deletions can be predicted based on the distance between the I-TevI and Cas domains.

FIG. 2A shows the workflow of the algorithm where a dataset comprising pairs of wild type and mutant target sites are generated from databases of known disease-causing mutations.

FIG. 2B includes sample output data from the wild type Muc4 gene.

FIG. 2C includes sample output data from a mutant Muc4 gene. The bolded row is found in both the wild type and mutant copies of the Muc4 gene sequence. The remaining cells in FIG. 2C constitute putative allele-specific TevSaCas9 targets in the Muc4 gene.

FIG. 3A is a diagram depicting TevSaCas9's target site in an oncogene (in bold) that spans a large insertion (in light gray).

FIG. 3B is an agarose gel electrophoresis result showing TevSaCas9's cleavage products from an in vitro cleavage assay of the wild type (WT) and mutant (MUT) copies of the Muc4 gene. As shown, TevSaCas9 targeted with the guide RNA of SEQ ID NO: 1685 has successfully cut the MUT substrate, but not the WT Muc4.

FIG. 4A, in particular, is an illustration of the EGFR L858R target site. The position of the mutation is denoted by an asterisk (“*”).

FIG. 4B is an agarose gel electrophoresis result showing the TevSaCas9 cleavage products from an in vitro cleavage assay of the wild type (WT) and mutant (MUT) copies of the EGFR gene. As shown, TevSaCas9 targeted with the guide RNA of SEQ ID NO: 1686 has preferentially cut the MUT EGFR L858R substrate over the WT EGFR substrate.

FIG. 4C is the result of a viability assay of treating the CLR-5908 cell line which contains mutant EGFR L858R and the NuLi-1 cell line which contains wild type EGFR with TevSaCas9 ribonucleoprotein complex targeted to EGFR L858R. As shown, TevSaCas9 target to EGFR L858R reduces the viability of the CRL-5908 cell line but not the NuLi-1 cell line demonstrating the allele-specific activity of TevSaCas9 In cells.

FIG. 5 depict a diagram of the mechanism by which TevCas is complexed with multiple guide RNAs and electroporated into cells to disrupt genes encoding the oncogene and insert a sequence coding for modified version of the oncogene, thereby restoring wild-type function.

### DETAILED DESCRIPTION OF THE DISCLOSURE

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed. As used herein, the singular forms “a,” “an,” and “the” include plural references unless the context clearly dictates otherwise. Any reference to “or” herein is intended to encompass “and/or” unless otherwise stated.

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference; thus, the inclusion of such definitions herein should not be construed to represent a substantial difference over what is generally understood in the art.

Within the framework of the present description and in the subsequent claims, except where otherwise indicated, all numbers expressing amounts, quantities, percentages, and so forth, are to be understood as being preceded in all instances by the term “about”. As used herein, the term “about” is defined as ±5%. Also, all ranges of numerical entities include all the possible combinations of the maximum and minimum numerical values and all the possible intermediate ranges therein, in addition to those specifically indicated hereafter.

The term “and/or” as used herein is defined as the possibility of having one or the other or both. For example, “A and/or B” provides for the scenarios of having just A or just B or a combination of A and B. If the claim reads A and/or B and/or C, the composition may include A alone, B alone, C alone, A and B but not C, B and C but not A, A and C but not B or all three A, B and C as components.

For convenience, certain terms employed in the specification, examples and appended claims are collected here. These definitions should be read in light of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e., to at least one) of the grammatical object of the article. The term “and/or” as used herein is defined as the possibility of having one or the other or both. For example, “A and/or B” provides for the scenarios of having just A or just B or a combination of A and B. If the claim reads A and/or B and/or C, the composition may include A alone, B alone, C alone, A and B but not C, B and C but not A, A and C but not B or all three A, B and C as components.

The term “donor DNA”, as used herein, refers to a DNA that, in whole or in part, differs from the original target DNA sequence, and can be incorporated into an oncogene to restore wild type or non-oncogenic function to the oncogene.

The term “flexible linker”, as used herein, refers to a situation when the RNA-guided Cas nuclease domain binds to the target DNA sequence, the amino acid linker domain ensures mobility of the I-TevI domain to allow for recognition, binding and cleaving of its target sequence under cell physiological conditions (typically: pH ˜7.2, temperature ˜37° C., [K+] ˜140 mM, [Na+] ˜5-15 mM, [Cl−] ˜4 mM, [Ca++] ˜0.0001 mM). The length of the amino acid linker can influence how many nucleotides are preferred between the Cas target site and the I-TevI target site. Certain amino acids in the linker may also make specific contacts with the DNA sequence targeted by TevCas. These linker-DNA contacts can affect the flexibility of the I-TevI domain. Substituting amino acids in the linker domain may affect the ability of the linker domain to make contact with DNA.

The term “including”, as used herein, is used to mean “including but not limited to”. “Including” and “including but not limited to” are used interchangeably.

The term “patient,” “individual,” “subject,” or “host” to be treated by the subject method may mean either a human or non-human animal. Non-human animals include companion animals (e.g. cats, dogs) and animals raised for consumption (i.e. food animals), such as cows, pigs, and chickens.

The term “pharmaceutically acceptable carrier” refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier can be “acceptable” in the sense of being compatible with the subject composition and its components and not injurious to the patient. Some examples of materials which may serve as pharmaceutically acceptable carriers include: (1) sugars, such as dextrose, lactose, glucose and sucrose; (2) starches, such as corn starch and potato starch; (3) cellulose, and its derivatives, such as microcrystalline cellulose, sodium carboxymethyl cellulose, methyl cellulose, ethyl cellulose, hydroxypropylmethyl cellulose (HPMC), and cellulose acetate; (4) glycols, such as propylene glycol; (5) polyols, such as glycerin, sorbitol, mannitol and polyethylene glycol; (6) esters, such as ethyl oleate, glyceryl behenate and ethyl laurate; (7) buffering agents, such as monobasic and dibasic phosphates, Tris/Borate/EDTA and Tris/Acetate/EDTA (8) pyrogen-free water; (9) isotonic saline; (10) Ringer's solution; (11) ethyl alcohol; (12) phosphate buffer solutions; (13) polysorbates; (14) polyphosphates; and (15) other non-toxic compatible substances employed in pharmaceutical formulations. The disclosed excipients may serve more than one function. For example, a solubilizing agent may also be a suspension aid, an emulsifier, a preservative, and the like.

In certain preferred embodiments, the pharmaceutically acceptable excipient is a crystalline bulking excipient. The terms “crystalline bulking excipient” or “crystalline bulking agent” as used herein means an excipient which provides bulk and structure to the lyophilization cake. These crystalline bulking agents are inert and do not react with the protein or nucleic acid. In addition, the crystalline bulking agents are capable of crystallizing under lyophilization conditions. Examples of suitable crystalline bulking agents include hydrophilic excipients, such as, water soluble polymers; sugars, such as mannitol, sorbitol, xylitol, glucitol, ducitol, inositiol, arabinitol, arabitol, galactitol, iditol, allitol, maltitol, fructose, sorbose, glucose, xylose, trehalose, allose, dextrose, altrose, lactose, glucose, fructose, gulose, idose, galactose, talose, ribose, arabinose, xylose, lyxose, sucrose, maltose, lactose, lactulose, fucose, rhamnose, melezitose, maltotriose, raffinose, altritol, their optically active forms (D- or L-forms) as well as the corresponding racemates; inorganic salts, both mineral and mineral organic, such as, calcium salts, such as the lactate, gluconate, glycerylphosphate, citrate, phosphate monobasic and dibasic, succinate, sulfate and tartrate, as well as the same salts of aluminum and magnesium; carbohydrates, such as, the conventional mono- and di-saccharides as well as the corresponding polyhydric alcohols; proteins, such as, albumin; amino acids, such as glycine; emulsifiable fats and polyvinylpyrrolidone. Crystalline bulking agents may be selected from any one of glycine, mannitol, dextran, dextrose, lactose, sucrose, polyvinylpyrrolidone, trehalose, glucose, or combination thereof. Particularly useful bulking agents include dextran.

The term “pharmaceutically-acceptable salts”, as used herein, is art-recognized and refers to the relatively non-toxic, inorganic and organic acid addition salts, or inorganic or organic base addition salts of compounds, including, for example, those contained in compositions of the present invention. Some examples of pharmaceutically-acceptable salts include: (1) calcium chlorides; (2) sodium chlorides; (3) sodium citrates; (4) sodium hydroxide; (5) sodium phosphates; (6) sodium ethylenediaminetetraacetic acid; (7) potassium chloride; (8) potassium phosphate; and (9) other non-toxic compatible substances employed in pharmaceutical formulations.

The term “substitution”, as used herein, refers to the replacement of an amino acid in a sequence with a different amino acid. As used herein, the shorthand X10Y indicates that amino acid Y has been “substituted” for amino acid X found in the 10^{th} position of the sequence. As an example, W26C denotes that amino acid Tryptophan-26 (Trp, W) is changed to a Cysteine (Cys). Similarly, the notation AA^{X} indicates that AA is an amino acid that replaced the amino acid found in the X position. As an example, Lys²⁶ denotes the replacement of the amino acid in the 26^{th} position in a sequence with Lysine. Use of either shorthand is interchangeable. In addition, use of the one- or three-letter abbreviations for an amino acid is also interchangeable.

The term “therapeutic agent”, as used herein refers to any chemical or biochemical moiety that is a biologically, physiologically, or pharmacologically active substance that acts locally or systemically in a subject. Examples of therapeutic agents, also referred to as “drugs,” are described in well-known literature references such as the Merck Index, the Physician's Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

The term, “hybridization,” as used herein, generally refers to and includes the capacity and/or ability of a first nucleic acid molecule to non-covalently bind (e.g., form Watson-Crick-base pairs and/or G/U base pairs), anneal, and/or hybridize to a second nucleic acid molecule under the appropriate or certain in vitro and/or in vivo conditions of temperature, pH, and/or solution ionic strength. Generally, standard Watson-Crick base pairing includes: adenine (A) pairing with thymidine (T); adenine (A) pairing with uracil (U); and guanine (G) pairing with cytosine (C). In some embodiments, hybridization comprises at least two nucleic acids comprising complementary sequences (e.g., fully complementary, substantially complementary, or partially complementary). In certain embodiments, hybridization comprises at least two nucleic acids comprising fully complementary sequences. In certain embodiments, hybridization comprises at least two nucleic acids comprising substantially complementary sequences (e.g., greater than about 75%, greater than about 80%, greater than about 85%, greater than about 90%, or greater than about 95% complementary). In certain embodiments, hybridization comprises at least two nucleic acids comprising partially complementary sequences (e.g., greater than about 40%, greater than about 50%, greater than about 60%, or greater than about 70% complementary). In certain embodiments, partially complementary sequences comprises one or more regions of fully or substantially complementary sequences. In certain embodiments, partially complementary sequences comprises one or more regions of fully or substantially complementary sequences, even if an overall complementarity is low (e.g., a total complementarity lower than about 50%, lower than about 40%, lower than about 30%, or lower than about 20%). The conditions appropriate for hybridization between two nucleic acids depend on the length of the nucleic acids and the degree of complementation, variables well known in the art. For example, the greater the degree of complementation between two nucleotide sequences, the greater the value of the melting temperature (Tm) for hybrids of nucleic acids having those sequences. For hybridizations between nucleic acids with short stretches of complementarity (e.g., complementarity over 35 or less, 30 or less, 25 or less, 22 or less, 20 or less, or 18 or less nucleotides) the position of mismatches becomes important (see Sambrook et al., supra, 11.7-11.8).

The term “complementary” or “complementarity,” as used herein, generally refers to a polynucleotide that includes a nucleotide sequence capable of selectively annealing to an identifying region of a target polynucleotide under certain conditions. As used herein, the term substantially complementary and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain embodiments, base-stacking and hydrophobic interactions can also contribute to duplex stability. Conditions under which a polynucleotide anneals to complementary or substantially complementary regions of target nucleic acids are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968). Annealing conditions will depend upon the particular application and can be routinely determined by persons skilled in the art, without undue experimentation. Hybridization generally refers to process in which two single-stranded polynucleotides bind non-covalently to form a stable double-stranded polynucleotide.

The temperature and solution salt concentration are generally recognized as factors facilitating hybridization, and may be adjusted as necessary according to factors such as length of the region of complementation and the degree of complementarity. Hybridization and washing conditions are well known and exemplified in Sambrook, J., Fritsch, E: F. and Maniatis, T. Molecular Cloning: A Laboratory Manual-Second Edition. Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the stringency of the hybridization. In some embodiments, hybridization is measured a under physiological temperature (e.g., 37 degrees Celsius) and salt concentrations (e.g., 0.15 molar or 0.9% salt in solution).

The term “treating”, as used herein, includes any effect, e.g., lessening, reducing, modulating, or eliminating, that results in the improvement of the condition, disease, disorder, and the like. As used herein, “treating” can include both prophylactic, and therapeutic treatment.

A “buffer” as used herein is any acid or salt combination which is pharmaceutically acceptable and capable of maintaining the composition of the present invention within a desired pH range. Buffers in the disclosed compositions maintain the pH in a range of about 2 to about 8.5, about 5.0 to about 8.0, about 6.0 to about 7.5, about 6.5 to about 7.5, or about 6.5. Suitable buffers include, any pharmaceutical acceptable buffer capable of maintaining the above pH ranges, such as, for example, acetate, tartrate phosphate or citrate buffers. In one embodiment, the buffer is a phosphate buffer. In another embodiment the buffer is an acetate buffer. In one embodiment the buffer is disodium hydrogen phosphate, sodium chloride, potassium chloride and potassium phosphate monobasic.

In the disclosed compositions the concentration of buffer is typically in the range of about 0.1 mM to about 1000 mM, about 0.2 mM to about 200 mM, about 0.5 mM to about 50 mM, about 1 mM to about 10 mM or about 6.0 mM.

As used herein, a stabilizer is a composition which maintains the chemical, biological or stability of the chimeric nuclease. Examples of stabilizing agent include polyols, which includes a saccharide, preferably a monosaccharide or disaccharide, e.g., glucose, trehalose, raffinose, or sucrose; a sugar alcohol such as, for example, mannitol, sorbitol or inositol, a polyhydric alcohol such as glycerin or propylene glycol or mixtures thereof and albumin.

A pharmaceutically acceptable salt is a salt which is suitable for administration to a subject, such as, a human. The chimeric nuclease of the present invention can have one or more sufficiently acidic proton that can react with a suitable organic or inorganic base to form a base addition salt. Base addition salts include those derived from inorganic bases, such as ammonium or alkali or alkaline earth metal hydroxides, carbonates, bicarbonates, and the like, and organic bases such as alkoxides, alkyl amides, alkyl and aryl amines, and the like. Such bases useful in preparing the salts of this invention thus include sodium hydroxide, potassium hydroxide, ammonium hydroxide, potassium carbonate, and the like. The chimeric nuclease of the present invention having a sufficiently basic group, such as an amine can react with an organic or inorganic acid to form an acid addition salt. Acids commonly employed to form acid addition salts from compounds with basic groups are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), et cetera. Sometimes a cell may not originate from a natural organism (e.g., a cell can be synthetically made, sometimes termed an artificial cell). In certain embodiments cells refers to human cells.

The terms “protein” and “polypeptide” can be used interchangeably to refer to a polymer of two or more amino acids joined by covalent bonds (e.g., an amide bond) that can adopt a three-dimensional conformation. In some embodiments, a protein or polypeptide comprises at least 10 amino acids, 15 amino acids, 20 amino acids, 30 amino acids or 50 amino acids joined by covalent bonds (e.g., amide bonds). In some embodiments, a protein comprises at least two amide bonds. In some embodiments, a protein comprises multiple amide bonds. In some embodiments, a protein comprises an enzyme, enzyme precursor proteins, regulatory protein, structural protein, receptor, nucleic acid binding protein, a biomarker, a member of a specific binding pair (e.g., a ligand or aptamer), or an antibody. In some embodiments, a protein can be a full-length protein (e.g., a fully processed protein having certain biological function). In some embodiments, a protein can be a variant or a fragment of a full-length protein. For example, in some embodiments, a Cas9 protein domain comprises an H840A amino acid substitution compared to a naturally occurring S. pyogenes Cas9 protein. A variant of a protein or enzyme, for example a variant reverse transcriptase, comprises a polypeptide having an amino acid sequence that is about 60% identical, about 70% identical, about 80% identical, about 90% identical, about 95% identical, about 96% identical, about 97% identical, about 98% identical, about 99% identical, about 99.5% identical, or about 99.9% identical to the amino acid sequence of a reference protein.

In some embodiments, a protein comprises a functional variant or functional fragment of a full-length wild type protein. A “functional fragment” or “functional portion”, as used herein, refers to any portion of a reference protein (e.g., a wild type protein) that encompasses less than the entire amino acid sequence of the reference protein while retaining one or more of the functions, e.g., catalytic or binding functions. For example, a functional fragment of a Cas or I-TevI protein can encompass less than the entire amino acid sequence of a wild type Cas or I-TevI protein, but retains the ability to catalyze the cleavage of a polynucleotide sequence. When the reference protein is a fusion of multiple functional domains, a functional fragment thereof can retain one or more of the functions of at least one of the functional domains. For example, a functional fragment of a Cas can encompass less than the entire amino acid sequence of a wild type Cas, but retains its DNA binding ability and lacks its nuclease activity partially or completely. In certain embodiments, functional fragments comprise one or more deletions from the N- or C-terminus of a protein, polypeptide or domain described herein. In certain embodiments, functional fragments comprise a deletion of 1, 2, 3, 4, 5, 6, 7, 8, 9, 10, 15, 20, Or 25 amino acid deletions from the N- or C-terminus of a protein, polypeptide or domain described herein.

A “functional variant” or “functional mutant”, as used herein, refers to any variant or mutant of a reference protein (e.g., a wild type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a Cas or I-TevI protein can comprise one or more amino acid substitutions compared to the amino acid sequence of a wild type Cas or I-TevI protein, but retains the ability to catalyze the cleavage of a polynucleotide sequence. When the reference protein is a fusion of multiple functional domains, a functional variant thereof can retain one or more of the functions of at least one of the functional domains. For example, in some embodiments, a functional fragment of a Cas9 can comprise one or more amino acid substitutions in a nuclease domain, e.g., a H840A amino acid substitution, compared to the amino acid sequence of a wild type Cas9, but retains the DNA binding ability and lacks the nuclease activity partially or completely.

The terms “homologous,” “homology,” or “percent homology” as used herein refer to the degree of sequence identity between an amino acid and a corresponding reference amino acid sequence, or a polynucleotide sequence and a corresponding reference polynucleotide sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences can exhibit 93%, 95% or 98% sequence identity to the reference nucleic acid sequence. For example, a “region of homology to a genomic region” can be a region of DNA that has a similar sequence to a given genomic region in the genome. A region of homology can be of any length that is sufficient to promote binding of a spacer, a primer binding site, or a protospacer sequence to the genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that the region of homology has sufficient homology to undergo binding with the corresponding genomic region.

The term “identity,” or “homology” as used interchangeable herein, may be to calculations of “identity,” “homology,” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences may be a function of the number of identical positions shared by the sequences (i.e., % homology=# of identical positions/total # of positions x 100). For example, a position in the first sequence may be occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine homology between two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. A non-limiting example of such a mathematical algorithm may be described in Karlin, S. and Altschul, S., Proc. Natl. Acad. Sci. USA, 90-5873-5877 (1993). Such an algorithm may be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. In another embodiment, the percent identity between two amino acid sequences can be accomplished using, for example, the GAP program in the GCG software package (Accelrys, Cambridge, UK).

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide or portion thereof. In some embodiments, the homology or identity is assessed over a functional portion or specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, I990. A publicly available, internet interface, for performing BLAST analyses is accessible through the National Center for Biotechnology Information. Additional known algorithms include those published in:

Smith & Waterman, “Comparison of Biosequences”, Adv. Appl. Math. 2:482, I981; Needleman & Wunsch, “A general method applicable to the search for similarities in the amino acid sequence of two proteins” J. Mol. Biol. 48:443, I970; Pearson & Lipman “Improved tools for biological sequence comparison”, Proc. Natl. Acad. Sci. USA 85:2444, I988; or by automated implementation of these or similar algorithms. Global alignment programs can also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16: 276-277), and the GGSEARCH program https://fasta.bioch.virginia.edu/fasta_www2/, which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, I998).

A skilled person understands that amino acid (or nucleotide) positions can be determined in homologous sequences based on alignment, for example, “H840” in a reference Cas9 sequence can correspond to H839, or another position in a Cas9 homolog.

The term “polynucleotide” or “nucleic acid molecule” can be any polymeric form of nucleotides, including DNA, RNA, a hybridization thereof, or RNA-DNA chimeric molecules. In some embodiments, a polynucleotide comprises cDNA, genomic DNA, mRNA, tRNA, rRNA, or microRNA. In some embodiments, a polynucleotide is double-stranded, e.g., a double-stranded DNA in a gene. In some embodiments, a polynucleotide is single-stranded or substantially single-stranded, e.g., single-stranded DNA or an mRNA. In some embodiments, a polynucleotide is a cell-free nucleic acid molecule. In some embodiments, a polynucleotide circulates in blood. In some embodiments, a polynucleotide is a cellular nucleic acid molecule. In some embodiments, a polynucleotide is a cellular nucleic acid molecule in a cell circulating in blood.

Polynucleotides can have any three-dimensional structure. The following are nonlimiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA, isolated RNA, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA).

In some embodiments, a polynucleotide comprises deoxyribonucleotides, ribonucleotides or analogs thereof. In some embodiments, a polynucleotide comprises modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

In some embodiments, a polynucleotide is composed of a specific sequence of four nucleotide bases: adenine (A); cytosine (C); guanine (G); thymine (T); and uracil (U) for thymine when the polynucleotide is RNA. In some embodiments, the polynucleotide can comprise one or more other nucleotide bases, such as inosine (I), which is read by the translation machinery as guanine (G).

In some embodiments, a polynucleotide can be modified. As used herein, the terms “modified” or “modification” refers to chemical modification with respect to the A, C, G, T and U nucleotides. In some embodiments, modifications can be on the nucleoside base and/or sugar portion of the nucleosides that comprise the polynucleotide. In some embodiments, the modification can be on the internucleoside linkage (e.g., phosphate backbone). In some embodiments, multiple modifications are included in the modified nucleic acid molecule. In some embodiments, a single modification is included in the modified nucleic acid molecule.

The term “mutation” as used herein refers to a change and/or alteration in an amino acid sequence of a protein or a nucleic acid sequence of a polynucleotide. Such changes and/or alterations can comprise the substitution, insertion, deletion and/or truncation of one or more amino acids, in the case of an amino acid sequence, and/or nucleotides, in the case of nucleic acid sequence, compared to a reference amino acid or a reference nucleic acid sequence. In some embodiments, the reference sequence is a wild-type sequence. In some embodiments, a mutation in a nucleic acid sequence of a polynucleotide encodes a mutation in the amino acid sequence of a polypeptide. In some embodiments, the mutation in the amino acid sequence of the polypeptide or the mutation in the nucleic acid sequence of the polynucleotide is a mutation associated with a disease state.

The term “subject” or “individual” can be used interchangeably. Its grammatical equivalents as used herein can refer to a human or a non-human. An individual can be a mammal. A human individual can be male or female. A human individual can be of any age. A individual can be a human embryo. A human individual can be a newborn, an infant, a child, an adolescent, or an adult. A human individual can be in need of treatment for a genetic disease or disorder. In some embodiments, a individual is suffering from, susceptible to, or at a risk of developing cancer.

The term “oncogene” refers to a gene that upon mutation, disruption, or overexpression can lead to uncontrolled cell division leading to the formation of a tumor or cancer. Before mutation the oncogene is known as a proto-oncogene. An “oncogenic mutation” is any mutation that leads to the transformation of a proto-oncogene to an oncogene. Oncogenes can have a non-oncogenic function restored to by editing and reverting the function of the gene to a wild-type or non-pathogenic state. Such editing may restore a wild-type nucleotide sequence or amino acid sequence, or a sequence (amino acid or nucleotide) that differs from wild-type but restores a wild-type or non-pathogenic function.

While specific embodiments of the disclosure's embodiments have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

The term “chimeric nuclease” refers to fusion protein comprising an I-TevI nuclease domain and a Cas nuclease domain. In some cases, the I-TevI nuclease domain and Cas nuclease domain are operably linked. A target gene of the chimeric nuclease can comprise a double stranded DNA molecule having two complementary strands: a first strand that can be referred to as a “coding strand”, and a second strand that can be referred to as a “non-coding strand.” In some embodiments, in a chimeric nuclease uses a guide RNA sequence that is complementary, substantially complementary to a specific sequence on the target strand. In some cases. The guide RNA hybridizes or substantially hybridizes to a specific sequence on the target strand. In some embodiments, the guide RNA sequence anneals with the target strand at the search target sequence.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

The present disclosure describes chimeric nucleases and method of targeting oncogenes with the chimeric nucleases. For example, if the chimeric nuclease cleaves at two sites, it cleaves out precise lengths of DNA (for example, approximately 30-38 bases depending on the sites targeted by I-TevI domain and Cas domain). Thus, a target site can be selected in a gene to generate a precise-length deletion that will knock the gene out-of-frame in a cell. In one embodiment of the described nucleases, a nuclease targets and cleaves a target oncogene.

The present disclosure describes methods of using the described nucleases to genetically engineer a cell population in order to target, contact, edit, silence, disrupt, restore, insert, modify, delete, or replace a nucleotides sequence at a genomic location. The disclosure describes forming a chimeric nuclease guide RNA complexes and administering the chimeric nuclease guide RNA complexes to cells. This administration can occur using one or more methods of electroporation or lipid mediated transfection (e.g., cationic lipids). Alternatively, a nucleic acid or plurality of nucleic acids encoding the guide RNA and/or the chimeric nuclease can be transferred into the cell using a method selected from electroporation, viral transduction, and or lipid mediated transfection can be utilized. In embodiments, where a genome sequence is to be added to alter the genome a donor DNA can also be administered to affect the insertion or alteration. The donor DNA can be suitably provided as a linearized DNA, plasmid DNA or a viral vector.

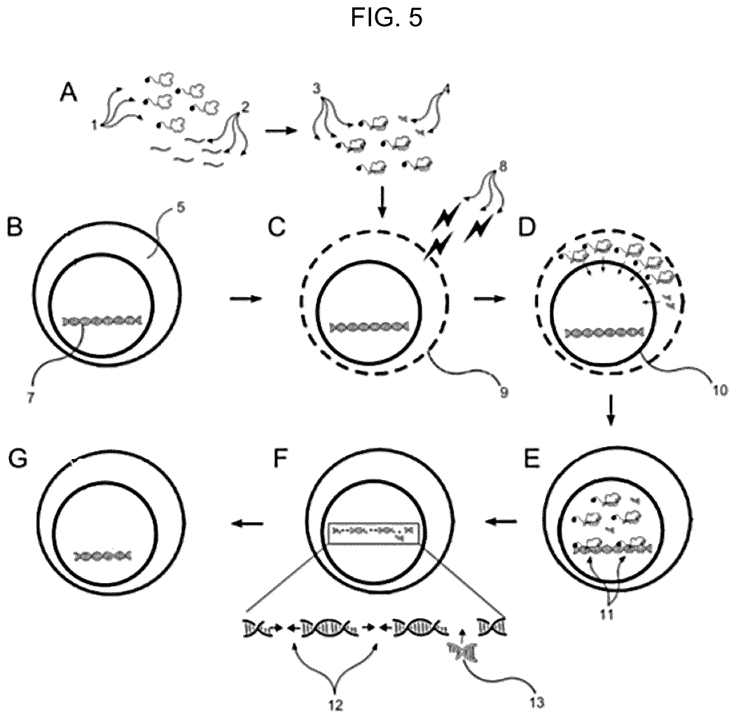

The methods described herein target oncogenes in a cell or an individual using a chimeric nuclease comprising an I-TevI nuclease domain and a Cas nuclease domain. As illustrated in FIG. 5A, purified chimeric nuclease (1) can be mixed complexed with multiple guide RNAs 2 to form ribonucleoprotein complex (3). Donor DNA (4) encoding an exogenous donor DNA containing complimentary ends to one or more of the sites targeted by the chimeric nuclease can be also mixed with the ribonucleoprotein complex (3). As shown in FIG. 5B, a population of cells (5) which encode for an oncogene expressed from the genomic DNA (7) can be exposed to the mixture of ribonucleoprotein complex (3) and donor DNA (4). An electrical pulse (8) can be applied to the mixture to permeabilize the cell membrane (9) (FIG. 5C). As depicted in FIG. 5D, the ribonucleoprotein complex (3) and donor DNA (4) can enter the cell through the permeabilized cell membrane. The ribonucleoprotein complex (3) can be targeted to the nucleus (10) through one or more nuclear localization sequences (“NLS”). As shown in FIG. 5E, the ribonucleoprotein complex (3) can bind to its target site on the genomic DNA (7). As depicted in FIG. 5F, the ribonucleoprotein complex (3) can cleave the genomic DNA (7) to create defined length deletions (12), or if compatible donor DNA (13) is present, can insert the donor DNA (13) into the cleaved site. The regions of the genomic DNA (7) deleted disrupt genes the oncogene (6) and the donor DNA (13) encodes for an modified version of the oncogene, thereby restoring wild-type function.

In addition, this disclosure is directed to a method of targeted gene disruption (e.g., insertion, edit, delete, modification or replacement) of all or a portion of a DNA sequence in the genome of human cells to knock genes out-of-frame, comprising: (a) exposing cells to the nuclease ex vivo; (b) applying an electric current of between 1000-2500V to the cell population to permeabilize the membrane to allow for the passage of the claimed nuclease into the cells. Other ranges of electric currents between 1000-1500V, 1501-1700V, 1701-1900V, 1901-2100V or 2101-2500V may also be applied. The nuclease may also be delivered to the cell using lipofection or polymer-based transfection or the use of a viral vector such as adeno-associated virus or lentivirus. The nuclease may further be delivered as a ribonucleoprotein complex, a DNA encoding the nuclease or as messenger RNA encoding the nuclease. In eukaryotic cells, the chimeric nucleases of this disclosure can target the nuclei of the cells through one or more nuclear-localization sequences (“NLS”). For the application of generating knockouts of oncogenes in cells, a mixture of nucleases can be applied to target one or more oncogenes in the population of cells. Specific guide RNAs to target the chimeric nuclease to a precise genomic location can be included with the nuclease, encoded by a nucleic acid, or a messenger RNA. For applications that target the replacement, repair, or insertion of a DNA into a genomic location, a donor nucleic acid may also be included either as an isolated and purified nucleic acid, by linear double stranded nucleic acid, by a plasmid or viral vector. A donor nucleic acid may be provided along with the nuclease and guide RNA or separately in separate formulation or delivered by a different method compared to the delivery of the nuclease and guide RNA. In the presence of a donor nucleic acid, the cell can insert the donor nucleic acid sequence (in whole or in part) between the two cleaved sites in the target genomic DNA using directed-ligation through non-homologous end joining.

The present disclosure is directed to chimeric nucleases comprising different combinations of an I-TevI domain and a Cas domain. In some embodiments, the chimeric nuclease further comprises a linker domain. In some embodiments, the chimeric nuclease further comprises a guide RNA.

Chimeric nucleases which target an oncogene can comprise (a) the I-TevI domain and the Cas domain; and (b) a guide RNA. In some embodiments, the guide RNA comprises an RNA sequence that hybridizes or is sufficiently complementary to at least a portion of a sequence selected from any one of SEQ ID NOs 1-683 or a combination thereof, or comprises a nucleotide sequence as set forth in SEQ ID NO: 1001-1686 or a combination thereof. In some embodiments, chimeric nucleases which target KRAS comprise (a) a chimeric nuclease as described above; and (b) a guide RNA that comprises the nucleic acid sequence that hybridizes to a target nucleic acid sequence at least 85%, 90%, 95%, 97%, 98%, or 99% identical to any one of SEQ ID NOs 37, 42, 51, 52, 62, 63, or 77. In some embodiments, chimeric nucleases which target PI3KCA comprise (a) a chimeric nuclease as described above; and (b) a guide RNA that comprises the nucleic acid sequence that hybridizes to a target nucleic acid sequence at least 85%, 90%, 95%, 97%, 98%, or 99% identical to any one of SEQ ID NOs 5, 6, 7, 8, 33, 202, 204, 209 or 210. In some embodiments, chimeric nucleases which target MUC-4 comprise (a) a chimeric nuclease as described above; and (b) a guide RNA that comprises the nucleic acid sequence that hybridizes to a target nucleic acid sequence at least 85%, 90%, 95%, 97%, 98%, or 99% identical to any one of SEQ ID NOs 676, 677, 678, 679 or 682. In some embodiments, chimeric nucleases which target EGFR comprise (a) a chimeric nuclease as described above; and (b) a guide RNA that comprises the nucleic acid sequence that hybridizes to a target nucleic acid sequence at least 85%, 90%, 95%, 97%, 98%, or 99% identical to any one of SEQ ID NOs 45, 130, 141, or 683.

Chimeric nucleases which target an oncogene comprise (a) the I-TevI domain and the Cas domain; and (b) a guide RNA. In some embodiments, the guide RNA comprises an RNA sequence with at least about 90%, 95%, 97%, 98%, or 99% identity or is identical to a sequence selected from any one of SEQ ID NOs 1001-1686, or a combination thereof. In some embodiments, chimeric nucleases which target KRAS comprise (a) a chimeric nuclease as described above; and (b) a guide RNA comprising an RNA sequence with at least about 90%, 95%, 97%, 98%, or 99% identity or is identical to a sequence selected from any one of SEQ ID NOs 1037, 1042, 1051, 1052, 1062, 1063, 1077, or a combination thereof. In some embodiments, chimeric nucleases which target PI3KCA comprise (a) a chimeric nuclease as described above; and (b) a guide RNA comprising an RNA sequence with at least about 90%, 95%, 97%, 98%, or 99% identity or is identical to a sequence selected from any one of SEQ ID NOs 1005, 1006, 1007, 1008, 1033, 1202, 1204, 1209, 1210, or a combination thereof. In some embodiments, chimeric nucleases which target MUC-4 comprise (a) a chimeric nuclease as described above; and (b) a guide RNA comprising an RNA sequence with at least about 90%, 95%, 97%, 98%, or 99% identity or is identical to a sequence selected from any one of SEQ ID NOs 1676, 1677, 1678, 1679, 1682, or a combination thereof. Chimeric nucleases which target EGFR comprise (a) a chimeric nuclease as described above; and (b) a guide RNA comprising an RNA sequence with at least about 90%, 95%, 97%, 98%, or 99% identity or is identical to a sequence selected from any one of SEQ ID NOs 1683, 1684, or a combination thereof.

In some embodiments, the chimeric nucleases are used to edit multiple genes simultaneously to generate multiple oncogene knockouts in a population of cells. For example, multiple chimeric nucleases can be used to target different oncogenes in an individual. In some embodiments, at least 1 chimeric nuclease is used to target an oncogenic mutation. In some embodiments, at least 2 chimeric nucleases is used to target an oncogenic mutation. In some embodiments, at least 3 chimeric nucleases is used to target an oncogenic mutation. In some embodiments, at least 4 chimeric nucleases is used to target an oncogenic mutation. In some embodiments, at least 5 chimeric nucleases is used to target an oncogenic mutation. In particular, the composition is directed to a mixture of the chimeric nucleases discussed above in the preceding paragraph in combination with a mixture of guide RNAs according to sequences SEQ ID NOs: 1001-1686 In an equimolar ratio to the chimeric nuclease. In another embodiment, the composition is directed to a mixture of the chimeric nucleases discussed above in the preceding paragraph in combination with a mixture of guide RNAs according to sequences SEQ ID NOs: 1001-1686 In an equimolar ration to the chimeric nuclease.

In some embodiments, a composition of other chimeric nucleases containing different combinations of an I-TevI domain and an RNA-guided nuclease domain. In particular, the composition is directed to chimeric nucleases of SEQ ID NOs: 730-736, 740-755, or 756, wherein the chimeric nuclease comprises a wildtype I-TevI domain or variant thereof, and a wildtype Cas domain or variant thereof.

The chimeric nucleases described herein can be formed from two different nucleases. The chimeric nucleases are useful for the ex vivo gene editing applications described herein and for in vivo applications.

### Chimeric Nucleases

The chimeric nuclease of the present disclosure may contain different combinations of an I-TevI domain and a Cas domain. In some embodiments, the Cas domain can be a Cas9 domain. In some embodiments, the Cas9 domain is derived from Staphylococcus aureus, Streptococcus pyogenes, Neisseria meningitidis, Campylobacter jejuni, Streptococcus pasteurianus, Clostridium cellulolyticum, or Geobacillus thermodenitrificans TI.

In some embodiments, the chimeric nuclease further comprises a linker domain. In some embodiments, the chimeric nuclease further comprises a guide RNA, wherein the guide RNA targets an oncogenic mutation. In some embodiments, the chimeric nuclease can be used to target the oncogenic mutation. In some embodiments, the oncogenic mutation is a single polynucleotide polymorphism or SNP. In some embodiments, the oncogenic mutation is an insertion of one or more nucleotides. In some embodiments, the oncogenic mutation is a substitution or deletion of 10 or less nucleotides. In some embodiments, the oncogenic mutation comprises a deletion of 10, 9, 8, 7, 6, 5, 4, 3, 2, or 1 nucleotides. In some embodiments, the oncogenic mutation is at least 85%, 90%, 95%, 97%, 98%, or 99% identical to a sequence set forth in any one of SEQ ID NOs: 1-628, or a combination thereof. In some embodiments, the oncogenic mutation occurs in at least one of ABL1, AFF4/MLLT11, AKT2, ALK, ALK/NPM, RUNX1 (AML1), RUNX1/MTG8 (ETO), AXL, BCL-2, BCL-3, BCL-6, BCR/ABL, MYC (c-MYC), MCF2 (DBL), DEK/NUP214, TCF3/PBX1, EGFR, MLLT11, ERG/FUS, ERBB2, ETS1, EWSR1/FLI1, CSF1R, FOS, FES, GLIl, GNAS (GSP), HER2/neu, TLX1, FGF4, IL3, FGF3 (INT-2), JUN, KIT, FGF4 (KS3), K-SAM, AKAP13, LCK, LMO1, LMO2, MYCL, LYL1, NFKB2, NFKB2/Cal, MAS1, MDM2, MLLT11, MOS, MUC4, RUNX1T1, MYB, MYH11/CBFB, NEU, MYCN, MCF2L (OST), PAX-5, PBX1/E2A, PIM1, PIK3CA, CCND1, RAFI, RARA/PML, HRAS, KRAS, NRAS, REL/NRG, RET, RHOM1, RHOM2, ROS1, SKI, SIS (aka PDGFB), SET/CAN, SRC, TAL1, TAL2, NOTCHI (TAN1), TIAM1, TSC2, or NTRK1. In some embodiments, the oncogenic mutation occurs in at least one of Muc4, PIK3CA, EGFR, or KRAS.

In some embodiments, the oncogenic mutation occurs in EGFR (e.g., UniProt accession number P00533). In some embodiments, the oncogenic mutation in EGFR is not a deletion in exon 19 of EGFR. In some embodiments, the oncogenic mutation in EGFR is at least one mutation corresponding to any one of P3L, S4A, GSA, T6A, A7P, A7D, L858R, V769_D770insASV, G8R, A10G, A13V, A16S, L18F, P20L, P20Q, A21S, A21T, A24T, K29T,T34M, T39M, D46E, R53K, E59K, E6K, Q18R, Q71R, T28A, T81A, Q83E, Q30E, E31Q, E84Q, E84D, E31D, A33V, A86V, L37F, L90F, N41S, N94S, V43M, V96M, R98Q, R45Q, Q52H, Q105H, R108G, R55G, R108K, R55K, G109A, G56A, M1 I1T, M58T, or a combination thereof. In some embodiments, the oncogenic mutation comprises a mutation in EGFR corresponding to L858R. In some embodiments, the oncogenic mutation in EGFR is a mutation corresponding to V769_D770insASV.

In some embodiments, the oncogenic mutation occurs in Muc4 (e.g., UniProt accession number Q99102). In some embodiments, Muc4 mutation is an in-frame deletion of exon 2 or an in-frame deletion of exon 3. In some embodiments, the Muc4 mutation is a mutation corresponding to any one of positions P1542, P1680, T1711, V1721, P1826, A1830, S3560, A1833, D2253, V2281, P3088, T3119, T3183, V3817, A3902, or any combination thereof. In some embodiments, the Muc4 mutation is selected from a mutation corresponding to P1542L, P1680S, T17111, V1721A, P1826H, A1830T, S3560S, A1833V, D2253H, V2281AM, P3088L, T3119T, T3183M, V3817A, A3902V, or a combination thereof.

In some embodiments, the oncogenic mutation occurs in KRAS (e.g., UniProt accession number P01116). In some embodiments, the KRAS mutation comprises a mutation corresponding to any one of positions A59, D119, D33, G21, G12, G13, Q61, A146, K117, or any combination thereof. In some embodiments, the KRAS mutation a mutation corresponding to any one of A59T, A59E, A59T, D119N, D33E, G21C, G12C, G12D, G12V, G12R, G12A, G12S, G13D, G13C, G13V, G13R, Q61R, Q61V, Q61L, Q61K, Q61H, Q61A, Q61P, Q61E, A146T, A146V, K117N, K117R, or a combination thereof.

In some embodiments, the oncogenic mutation occurs in PIK3CA (e.g., UniProt accession number P42336). In some embodiments, the PIK3CA mutation is a mutation corresponding to positions H1047, E542, E545, N345, C1636, G1624, G1633, A3140, C3075, A1634, A1173, or a combination thereof. In some embodiments, the PIK3CA mutation is a mutation corresponding to any one of H1047R, H1047L, E542K, E545K, N345K, C1636A, G1624A, G1633A, A3140T, A3140G, C3075T, A1634C, A1173G, or a combination thereof.

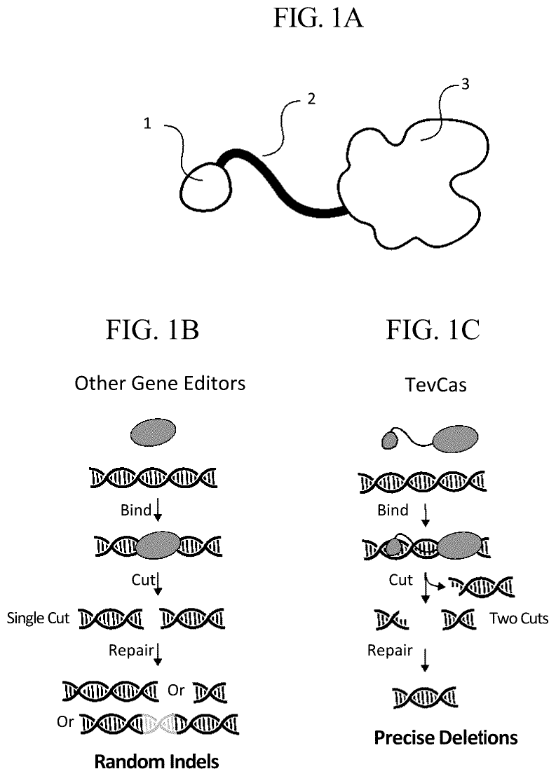

The present disclosure describes chimeric nucleases and methods of using the chimeric nucleases to target oncogenic mutations. Cleavage with existing single-cut endonucleases can leave compatible DNA ends in the target site (FIG. 1B), whereas cleavage by a chimeric nuclease can leave a blunt end at the Cas site and a 3′-overhang at the I-TevI site (FIG. 1C). When the chimeric nuclease cleaves at two sites, it cleaves out precise lengths of DNA (for example, approximately 30-40 bases depending on the sites targeted by I-TevI and SaCas9). The methods described herein may generate precise deletions of at least about 30, 31, 32, 33, 34, 35, 36, 37, 38, 39, or at most about 40 nucleotides from a genome. The chimeric nucleases described herein may generate precise deletions of at least about 30, 31, 32, 33, 34, 35, 36, 37, 38, 39, or at most about 40 nucleotides from a genome. Thus, a target site can be selected in a gene to generate a precise-length deletion that will knock an oncogene out-of-frame in a cell or organism. In some embodiments, a chimeric nuclease of the present disclosure may require four elements in the target site to bind and cut: 1) an I-TevI site; 2) about 14-19 nucleotides of spacer sequence; 3) about 19- to 21-nucloetides of protospacer sequence and 4) a protospacer adjacent motif (PAM), where N is any nucleotide, R is the nucleotide A or G and K is the nucleotide T or G. Exemplary I-TevI motifs and PAMs are given in Table 1 and Table 2, respectively. Given the unique target site requirement of the chimeric nuclease, target sites can be selected where an oncogenic mutation changes one of these elements to preferentially or selectively target the mutation. In one embodiment of the described chimeric nucleases generates a precise-length deletion in an oncogene but not in the wild type gene.

Table 1 describes different I-TevI variants. Different mutations to the I-TevI can alter the specificity of the binding site and changing the consensus sequence.

### Oncogenic Mutations

Oncogenic mutations other than those described can also be allele-specific targets of chimeric nucleases. Other allele-specific oncogenic mutations can be targeted as a result of a change in the binding site for the I-TevI domain, a change in the spacer DNA sequence between the I-TevI site and Cas site, a change in the Cas protospacer sequence or a change in the Cas protospacer adjacent motif sequence.

### Epidermal Growth Factor-EGFR

In some embodiments, the oncogenic mutation occurs in EGFR. In some embodiments, the oncogenic mutation in EGFR is not a deletion in exon 19 of EGFR. In some embodiments, the oncogenic mutation in EGFR is a mutation corresponding to L858R. In some embodiments, the guide RNA hybridizes to a target nucleotide sequence set forth in SEQ ID NO: 45, 130, or 141, or comprises a nucleotide sequence as set forth in SEQ ID NO: 1045, I130, I141, or 1686. In some embodiments, the guide RNA comprises the nucleic acid sequence that hybridizes to a target nucleic acid sequence at least 85%, 90%, 95%, 97%, 98%, or 99% identical to any one of SEQ ID NO: 45, 130, or 141, or comprises a nucleotide sequence at least 85%, 90%, 95%, 97%, 98%, or 99% identical to any one of SEQ ID NO: 1045, I130, I141, or 1686. In some embodiments, the oncogenic mutation in EGFR is a mutation corresponding to V769_D770insASV. In some embodiments, the guide RNA hybridizes to a target nucleotide sequence set forth in SEQ ID NO: 683, or comprises a nucleotide sequence as set forth in SEQ ID NO: 1683 or 1684. In some embodiments, the guide RNA comprises the nucleic acid sequence that hybridizes to a target nucleic acid sequence at least 85%, 90%, 95%, 97%, 98%, or 99% identical to any one of SEQ ID NO: 683, or comprises a nucleotide sequence at least 85%, 90%, 95%, 97%, 98%, or 99% identical to any one of SEQ ID NO: 1683 or 1684.

### Mucin 4—Muc 4

Mucin 4 (MUC-4) is a mucin protein that in humans is encoded by the MUC4 gene. Like other mucins, MUC-4 is a high-molecular weight glycoprotein. MUC-4 belongs to the human mucin family that is membrane-anchored and can range in molecular weight from 550 to 930 kDa for the actual protein, and up to 4,650 kDa with glycosylation. MUC4 can also be referred to as ASGP, HSA276359, MUC-4, or mucin 4.

MUC4 is an O-glycoprotein that can reach up to 2 micrometers outside the cell. MUC4 mucin consists of a large extracellular alpha subunit that is heavily glycosylated and a beta subunit that is anchored in the cell membrane and extends into the cytosol. The beta subunit is considered an oncogene, whose role in cancer is increasingly being recognized particularly due to its involvement in signaling pathways, particularly with ErbB2 (Her2).

The two subunits of MUC4 are transcribed from a single gene made of 25 exons and with its exon/intron structure identical to that of the mouse gene. Over 24 splice variants have been found for MUC4 using commercial mRNAs or total RNAs extracted from cancer cell lines.

MUC-4 is thought to play a role in cancer progression by repressing apoptosis and consequently increasing tumor cell proliferation. The molecular mechanism is thought to be through a MUC-4 complex with ERBB2 receptors, which alters downstream signaling and down regulates CDKN1B. The beta subunit of MUC-4 appears to serve as a ligand that causes the phosphorylation of ErbB2, but does not activate the MAPK or AKT pathways. MUC-4 may also affect HER2 signaling, and result in its stabilization. As a mucin, MUC-4 also alters adhesive properties of the cell. When overexpressed, the disorganization of mucins may reduce adhesion to other cells as well as the extracellular matrix, promoting cancer cell migration and metastasis.

The chimeric nuclease of the present disclosure can target an oncogenic mutation. Such as Muc-4. In some embodiments, the oncogenic mutation occurs in Muc4. In some embodiments, Muc4 mutation is an in-frame deletion of exon 2 or an in-frame deletion of exon 3. In some embodiments, the Muc4 mutation is a mutation corresponding to any one of positions P1542, P1680, T1711, V1721, P1826, A1830, S3560, A1833, D2253, V2281, P3088, T3119, T3183, V3817, A3902, or any combination thereof. In some embodiments, the Muc4 mutation is a mutation corresponding to P1542L, P1680S, T1711I, V1721A, P1826H, A1830T, 535605, A1833V, D2253H, V2281AM, P3088L, T3119T, T3183M, V3817A, A3902V, or a combination thereof. In some embodiments, the guide RNA hybridizes to a target nucleotide sequence set forth in SEQ ID NO: 676, 677, 678, 679 or 682, or comprises a nucleotide sequence as set forth in SEQ ID NO: 1676, I677, I678, I679, I682, or 1685. In some embodiments, the guide RNA comprises the nucleic acid sequence that hybridizes to a target nucleic acid sequence at least 85%, 90%, 95%, 97%, 98%, or 99% identical to any one of SEQ ID NO: 676, 677, 678, 679 or 682, or comprises a nucleotide sequence at least 85%, 90%, 95%, 97%, 98%, or 99% identical to any one of SEQ ID NO: 1676, I677, I678, I679, I682, or 1685.

### K-Ras—KRAS

K-Ras is a part of the RAS/MAPK pathway. KRAS is also known as KRAS, C—K—RAS, CFC2, K-RAS2A, K-RAS2B, K-RAS4A, K-RAS4B, KI-RAS, KRAS1, KRAS2, NS, NS3, RALD, RASK2, K-ras, KRAS proto-oncogene, GTPase, c-Ki-ras2, OES, c-Ki-ras, K-Ras 2, ‘C—K-RAS, K-Ras, Kirsten Rat Sarcoma virus, or Kirsten Rat Sarcoma virus. The protein relays signals from outside the cell to the cell's nucleus. These signals instruct the cell to grow and divide (proliferate) or to mature and take on specialized functions (differentiate). It is called KRAS because it was first identified as a viral oncogene in the Kirsten RAt Sarcoma virus. The oncogene identified was derived from a cellular genome, so KRAS, when found in a cellular genome, is called a proto-oncogene.

KRAS acts as a molecular on/off switch. Once it is allosterically activated, it recruits and activates proteins necessary for the propagation of growth factors, as well as other cell signaling receptors like c-Raf and PI 3-kinase. KRAS upregulates the GLUT1 glucose transporter, thereby contributing to the Warburg effect in cancer cells. KRAS binds to GTP in its active state. It also possesses an intrinsic enzymatic activity which cleaves the terminal phosphate of the nucleotide, converting it to GDP. Upon conversion of GTP to GDP, KRAS is deactivated. The rate of conversion is usually slow but can be increased dramatically by an accessory protein of the GTPase-activating protein (GAP) class, for example RasGAP. In turn, KRAS can bind to proteins of the Guanine Nucleotide Exchange Factor (GEF) class (such as SOS1), which forces the release of bound nucleotide (GDP). Subsequently, KRAS binds GTP present in the cytosol and the GEF is released from ras-GTP.

In some embodiments, the oncogenic mutation occurs in KRAS. In some embodiments, the KRAS mutation comprises a mutation corresponding to any one of positions A59, D119, D33, G21, G12, G13, Q61, A146, K117, or any combination thereof. In some embodiments, the KRAS mutation is a mutation corresponding to any one of A59T, A59E, A59T, D119N, D33E, G21C, G12C, G12D, G12V, G12R, G12A, G12S, G13D, G13C, G13V, G13R, Q61R, Q61V, Q61L, Q61K, Q61H, Q61A, Q61P, Q61E, A146T, A146V, K117N, K117R, or a combination thereof. In some embodiments, the guide RNA hybridizes to a target nucleotide sequence set forth in SEQ ID NO: 37, 42, 51, 52, 62, 63, or 77, or comprises a nucleotide sequence as set forth in SEQ ID NO: 1042, 1051, 1052, 1062, 1063, or 1077. In some embodiments, the guide RNA comprises the nucleic acid sequence that hybridizes to a target nucleic acid sequence at least 85%, 90%, 95%, 97%, 98%, or 99% identical to any one of SEQ ID NO: 37, 42, 51, 52, 62, 63, or 77, or comprises a nucleotide sequence at least 85%, 90%, 95%, 97%, 98%, or 99% identical to any one of SEQ ID NO: 1037, 1042, 1051, 1052, 1062, 1063, or 1077. Phosphatidylinositol 3-kinase catalytic subunit PIK3CA

Phosphatidylinositol 3-kinase catalytic subunit (PIK3CA) is one of the most common mutated genes in breast cancer and has been found to important in a number of cancer types. An integral part of the PI3K pathway, PIK3CA has long been described as an oncogene, with two main hotspots for activating mutations, the 542/545 region of the helical domain, and the 1047 region of the kinase domain.

Phosphatidylinositol-4,5-bisphosphate 3-kinase (also called phosphatidylinositol 3-kinase (PI3K)) is composed of an 85 kDa regulatory subunit and a 110 kDa catalytic subunit (PIK3CA). The protein encoded by this gene represents the catalytic subunit, which uses ATP to phosphorylate phosphatidylinositols (PtdIns), PtdIns4P and Ptdlns (4, 5) P2. PIK3CA can also be referred to as CLOVE, CWS5, MCAP, MCM, MCMTC, PI3K, p¹10-alpha, PI3K-alpha, phosphatidylinositol-4,5-bisphosphate 3-kinase catalytic subunit alpha, CLAPO, CCM4.

In some embodiments, the oncogenic mutation occurs in PIK3CA. In some embodiments, the PIK3CA mutation is a mutation corresponding to any one of positions H1047, E542, E545, N345, C1636, G1624, G1633, A3140, C3075, A1634, A1173, or a combination thereof. In some embodiments, the PIK3CA mutation is a mutation corresponding to any one of positions H1047R, H1047L, E542K, E545K, N345K, C1636A, G1624A, G1633A, A3140T, A3140G, C3075T, A1634C, A1173G, or a combination thereof. In some embodiments, the guide RNA hybridizes to a target nucleotide sequence set forth in SEQ ID NO: 5, 6, 7, 8, 33, 202, 204, 209 or 210, or comprises a nucleotide sequence as set forth in SEQ ID NO: 1005, 1006, 1007, 1008, 1033, 1202, 1204, 1209, or 1210. In some embodiments, the guide RNA comprises the nucleic acid sequence that hybridizes to a target nucleic acid sequence at least 85%, 90%, 95%, 97%, 98%, or 99% identical to any one of SEQ ID NO: 5, 6, 7, 8, 33, 202, 204, 209 or 210, or comprises a nucleotide sequence at least 85%, 90%, 95%, 97%, 98%, or 99% identical to any one of SEQ ID NO: 1005, 1006, 1007, 1008, 1033, 1202, 1204, 1209, or 1210.

### I-TevI Nuclease

The chimeric nuclease of the present disclosure may comprise an I-TevI nuclease domain. An unmodified full-length I-TevI protein comprises the sequence according SEQ ID NO: 702.

(SEQ ID NO: 702)
MGKSGIYQIKNTLNNKVYVGSAKDFEKRWKRHFKDLEKGCHSSIKLQRS
FNKHGNVFECSILEEIPYEKDLIIERENFWIKELNSKINGYNIADATFG
DTCSTHPLKEEIIKKRSETVKAKMLKLGPDGRKALYSKPGSKNGRWNPE
THKFCKCGVRIQTSAYTCSKCRN.

The sequence provided by SEQ ID NO: 700, 702, or 704 is a wild-type version of I-TevI except for a glycine insertion at position 2 that increases protein stability and prevents N-terminal degradation. With respect to specific substitutions referred to herein, the numbering corresponds to the wild-type version of the protein lacking the glycine stabilization. Thus, in the stabilized version of I-TevI the lysine at position 27 of SEQ ID NO: 700, 702, or 704 is referred to as K26 corresponding to the wild-type position without the glycine at position 2. There are several I-TevI substitutions to the I-TevI domain known to have little effect on I-TevI nuclease activity. Nuclease activity of I-TevI can be assayed for by mixing a chimeric nuclease containing the I-TevI domain with linear DNA containing a known I-TevI target and resolving the products of the cleavage reaction on an agarose gel. Products of the predicted size will be present if the I-TevI nuclease is active.

The chimeric nuclease of the present disclosure can comprise an I-TevI nuclease domain. In some embodiments, the I-TevI nuclease domain is derived from Enterobacteria Phage T4. The I-TevI domain can comprise a 93-amino acid I-TevI domain of the Enterobacteria Phage T4 according to the following sequence:

(SEQ ID NO: 700)
MGKSGIYQIKNTLNNKVYVGSAKDFEKRWKRHFKDLEKGCHSSIKLQRS
FNKHGNVFECSILEEIPYEKDLIIERENFWIKELNSKINGYNIA.

In some embodiments, exemplary I-TevI nuclease domain are shown in SEQ ID NO: 700. In some embodiments, the mutation can correspond to any one of SEQ ID NO: 700.

In some embodiments, the I-TevI nuclease domain can comprise at least one mutation as compared to SEQ ID NO: 700, 702, or 704. In some embodiments, the I-TevI nuclease domain comprises a mutation corresponding to any one of the positions selected from any one of T11, V16, N14, E25, K26, R27, E36, K37, G38, C39, S41, L45, F49, I60, E81, or a combination thereof. In some embodiments, the I-TevI nuclease domain comprises a mutation corresponding to any one of T11V, V16I, N14G, E25D, K26R, R27A, E36S, K37N, G38N, C39V, S41H, L45F, F49Y, I60V, E81I, or a combination thereof. In some embodiments, the I-TevI nuclease domain comprises at least a K26R mutation as compared to a wild-type sequence. In some embodiments, the I-TevI nuclease domain comprises an amino acid sequence having at least 75%, 80%, 85%, 90%, 95%, 96%, 97%, 98%, 99%, or 100% sequence identity to SEQ ID NO: 700, 702, or 704.

Other versions of the I-TevI nuclease domain might contain different combinations of mutations to alter the site targeted by the I-TevI domain or the activity of the I-TevI domain, including mutations that alter the sequence recognized by I-TevI, such as K26 and/or C39. Other versions of the nuclease might substitute the I-TevI domain with other GIY-YIG nuclease domains, such as I-BmoI, Eco29kI, etc. Some versions of I-TevI do not contain Metl as a result of processing when expressed in E. coli.

The unmodified full-length I-TevI nuclease comprises a nuclease domain, comprising position 1-93 and a linker domain comprising position 94-169. The positions of the mutations correspond to the positions according to the unmodified full-length I-TevI nuclease, or SEQ ID NO: 700, 702, or 704.

Table 1 Summarizes a List of Exemplary Wildtype I-TevI and its Variants. In Addition. The Table Notes the Different Cleavage Motifs that are Used by the Different I-TevI Proteins.

TABLE 1
Exemplary I-TevI Variants and Cleavage motifs

| Protein Name | Variant | Motif |
|---|---|---|
| I-TevI | Wild-type | 5′-CAACG-3′ (SEQ ID NO: 800); |
| | | 5′-CGAAG-3′ (SEQ ID NO: 801) |
| I-TevI | K26R | 5′-CAACG-3′ (SEQ ID NO: 802); |
| | | 5′-CAAGG-3′ (SEQ ID NO: 803); |
| | | 5′-CGAAG-3′ (SEQ ID NO: 804) |
| I-TevI | T95S | 5′-CAACG-3′ (SEQ ID NO: 805); |
| | | 5′-CAAGG-3′ (SEQ ID NO: 806); |
| | | 5′-CCCCG-3′ (SEQ ID NO: 807); |
| | | 5′-CGAAG-3′ (SEQ ID NO: 808); |
| | | 5′-CGCCG-3′ (SEQ ID NO: 809); |
| | | 5′-CGGAG-3′ (SEQ ID NO: 810); |
| | | 5′-CTGGG-3′ (SEQ ID NO: 811) |
| I-TevI | Q158R | 5′-CAACG-3′ (SEQ ID NO: 812); |
| | | 5′-CAAGG-3′ (SEQ ID NO: 813); |
| | | 5′-CCCCG-3′ (SEQ ID NO: 814); |
| | | 5′-CGAAG-3′ (SEQ ID NO: 815); |
| | | 5′-CGGAG-3′ (SEQ ID NO: 816); |
| | | 5′-TAACG-3′ (SEQ ID NO: 817) |
| I-TevI | K26R/T95S | 5′-CAACG-3′ (SEQ ID NO: 818); |
| | | 5′-CAAGG-3′ (SEQ ID NO: 819); |
| | | 5′-CCCCG-3′ (SEQ ID NO: 820); |
| | | 5′-CGAAG-3′ (SEQ ID NO: 821); |
| | | 5′-CGCCG-3′ (SEQ ID NO: 822); |
| | | 5′-CGGAG-3′ (SEQ ID NO: 823); |
| | | 5′-CTGGG-3′ (SEQ ID NO: 824) |
| I-TevI | K26R/Q158R | 5′-CAACG-3′ (SEQ ID NO: 825); |
| | | 5′-CAAGG-3′ (SEQ ID NO: 826); |
| | | 5′-CCAGG-3′ (SEQ ID NO: 827); |
| | | 5′-CCCCG-3′ (SEQ ID NO: 828); |
| | | 5′-CGAAG-3′ (SEQ ID NO: 829); |
| | | 5′-CGGAG-3′ (SEQ ID NO: 830); |
| | | 5′-CTGGG-3′ (SEQ ID NO: 831); |
| | | 5′-TAACG-3′ (SEQ ID NO: 832) |
| I-TevI | T95S/Q158R | 5′-CAACG-3′ (SEQ ID NO: 833); |
| | | 5′-CAAGG-3′ (SEQ ID NO: 834); |
| | | 5′-CACGG-3′ (SEQ ID NO: 835); |
| | | 5′-CCCAG-3′ (SEQ ID NO: 836); |
| | | 5′-CCCCG-3′ (SEQ ID NO: 837); |
| | | 5′-CGAAG-3′ (SEQ ID NO: 838); |
| | | 5′-CGCAG-3′ (SEQ ID NO: 839); |
| | | 5′-CGCCG-3′ (SEQ ID NO: 840); |
| | | 5′-CGCTG-3′ (SEQ ID NO: 841); |
| | | 5′-CGGAG-3′ (SEQ ID NO: 842); |
| | | 5′-CTCGG-3′ (SEQ ID NO: 843); |
| | | 5′-CTGGG-3′ (SEQ ID NO: 844); |
| | | 5′-AAACG-3′ (SEQ ID NO: 845); |
| | | 5′-GAACG-3′ (SEQ ID NO: 846) |
| I-TevI | K26R/T95S/ | 5′-CAACG-3′ (SEQ ID NO: 847); |
| | Q158R | 5′-CAAGG-3′ (SEQ ID NO: 848); |
| | | 5′-CACGG-3′ (SEQ ID NO: 849); |
| | | 5′-CCCAG-3′ (SEQ ID NO: 850); |
| | | 5′-CCCCG-3′ (SEQ ID NO: 851); |
| | | 5′-CGAAG-3′ (SEQ ID NO: 852); |
| | | 5′-CGCAG-3′ (SEQ ID NO: 853); |
| | | 5′-CGCCG-3′ (SEQ ID NO: 854); |
| | | 5′-CGCTG-3 (SEQ ID NO: 855)′; |
| | | 5′-CGGAG-3′ (SEQ ID NO: 856); |
| | | 5′-CTGGG-3′ (SEQ ID NO: 857); |
| | | 5′-TAACG-3′ (SEQ ID NO: 858) |
| I-TevI | V117F | Improved binding to cleavage site |
| I-TevI | V117F/K135R/ | Relaxes spacer sequence contact |
| | N140S | requirement |

### Linker Domain

The chimeric nucleases of the present disclosure may further comprise a linker domain. The linker may comprise a flexible amino acid linker comprising from at least about 10, 20, 30, 40, 50, 60, 70, 80, 90, or 100 amino acids. The linker may comprise a flexible amino acid linker comprising from no more than 10, 20, 30, 40, 50, 60, 70, 80, 90, or 100 amino acids. In some embodiments, the linker domains can be unstructured or comprise a Gly-Ser linker. Longer linkers generally can relax the 14-19 base pair I-TevI spacing requirement in the target site, whereas shorter linkers generally restrict it. Useful linkers include, but are not limited to, glycine-serine polymers, including for example (GS) n (SEQ ID NO: 900), (GSGGS) n (SEQ ID NO: 901), (GGGGS)n (SEQ ID NO: 902), and (GGGS)n (SEQ ID NO: 903), where n is an integer of at least one, glycine-alanine polymers, alanine-serine polymers, and other flexible linkers. Exemplary, linkers for linking antibody fragments or single chain variable fragments can include AAEPKSS (SEQ ID NO: 904), AAEPKSSDKTHTCPPCP (SEQ ID NO: 904), GGGG (SEQ ID NO: 905), or GGGGDKTHTCPPCP (SEQ ID NO: 906). Alternatively, a variety of non-proteinaceous polymers, including but not limited to polyethylene glycol (PEG), polypropylene glycol, polyoxyalkylenes, or copolymers of polyethylene glycol and polypropylene glycol, may find use as linkers, that is may find use as linkers.In some embodiments, the I-TevI nuclease domain is joined to a Cas domain by a linker domain. The linker domain may comprise the I-TevI linker (amino acids 93-169 of SEQ ID NO: 701). In some embodiments, the linker comprises an amino acid sequence set forth

(SEQ ID NO: 703)
DATFGDTCSTHPLKEEIIKKRSETVKAKMLKLGPDGRKALYSKPGSKNGR
WNPETHKFCKCGVRIQTSAYTCSKCRNGGSGGS.

In some embodiments, exemplary linkers are shown in SEQ ID NO: 702 or 704. In some embodiments, the mutation can correspond to any one of SEQ ID NO: 702 or 704.

In some embodiments, the linker comprises a mutation corresponding to a position selected from any one of T95, S101, A119, K120, K135, P126, D127, N140, T147, Q158, A161, V117, S165, or a combination thereof. In some embodiments, the linker comprises a mutation corresponding to any one of T95S, S101Y, A119D, K120N, K135N, K135R, P126S, D127K, N140S, T147I, Q158R, A161V, V117F, S165G, or a combination thereof. In some embodiments, the linker comprises an amino acid sequence having at least 75%, 80%, 85%, 90%, 95%, 96%, 97%, 98%, 99%, or 100% sequence identity to SEQ ID NO: 701, 702, 703, or 704. In some embodiments, the linker comprises a mutation corresponding to K135R and/or N140S. In some embodiments, the linker comprises a mutation corresponding to V117F. In some embodiments, the linker comprises a mutation corresponding to V117F, K135R, and/or N140S.

### Cas Protein Domains

CRISPR systems generally contain two components: a guide RNA (gRNA or sgRNA) and a CRISPR-associated endonuclease (Cas protein). In nature, CRISPR/CRISPR-associated (Cas) systems provide bacteria and archaea with adaptive immunity against viruses and plasmids by using CRISPR RNAs (crRNAs) to guide the silencing of invading nucleic acids. The CRISPR-Cas is an RNA-mediated adaptive defense system that relies on small RNA molecules for sequence-specific detection and silencing of foreign nucleic acids. CRISPR-Cas systems are composed of cas genes organized in operon(s) and CRISPR array(s) consisting of genome-targeting sequences (termed spacers).

CRISPR-Cas systems can generally refer to and include an enzyme system that includes a guide RNA sequence that contains a nucleotide sequence complementary or substantially complementary to a region of a target polynucleotide (e.g., a template nucleic acid such a HSV genomic DNA), and a protein with nuclease activity. CRISPR-Cas systems can include Type I CRISPR-Cas system, Type II CRISPR-Cas system, Type III CRISPR-Cas system, and derivatives thereof. CRISPR-Cas systems include engineered and/or programmed nuclease systems derived from naturally accruing CRISPR-Cas systems. CRISPR-Cas systems may contain engineered and/or mutated Cas proteins. In certain embodiments, nucleases generally refer to enzymes capable of cleaving the phosphodiester bonds between the nucleotide subunits of nucleic acids. In some embodiments, endonucleases are generally capable of cleaving the phosphodiester bond within a polynucleotide chain.

In some embodiments, the CRISPR-Cas system used herein can be a type I, a type II, or a type III system. Non-limiting examples of suitable CRISPR-Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, Cas12, CasF, CasG, CasH, CasX, CasΦ, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CasX, Csx3, Csz1, Csx15, Csf1, Csf2, Csf3, Csf4, and Cu1966. In some embodiments, the CRISPR-Cas protein or endonuclease is Cas9. In some embodiments, the CRISPR-Cas protein or endonuclease is Cas12. In some embodiments, the CRISPR-Cas protein or endonuclease is CasX.

In some embodiments, the Cas9 protein can be from or derived from: Staphylococcus aureus, Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonfex degensii, Caldicelulosiruptor becscii, Candidatus desulforudis, Clostridium botulinum, Clostridium difficile, Fine goldia magna, Natranaerobius thermophilus, Pelotomaculum the rmopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In some embodiments, the CRISPR-Cas-like protein can be a wild type CRISPR-Cas protein, a modified CRISPR-Cas protein. In some embodiments, the CRISPR-Cas-like protein can be modified to increase nucleic acid binding affinity and/or specificity, alter an enzymatic activity, and/or change another property of the protein. For example, nuclease (i.e., DNase, RNase) domains of the CRISPR-Cas-like protein can be modified, deleted, or inactivated. Alternatively, in some embodiments, the CRISPR-Cas-like protein can be truncated to remove domains that are not essential for the function of the Cas protein. In some embodiments, the CRISPR-Cas-like protein can also be truncated or modified to optimize the activity of the effector domain of the Cas protein.

In some embodiments, the CRISPR-Cas-like protein can be derived from a wild type Cas protein or fragment thereof. In certain embodiments, the CRISPR-Cas-like protein is a modified Cas9 protein. For example, the amino acid sequence of the Cas9 protein can be modified to alter one or more properties (e.g., nuclease activity, affinity, stability, etc.) of the protein relative to wild-type or another Cas protein. Alternatively, in some embodiments, domains of the Cas9 protein not involved in RNA-guided cleavage can be eliminated from the protein such that the modified Cas9 protein is smaller than the wild-type Cas9 protein.

The chimeric nuclease of the present disclosure can comprise a Cas domain. In some embodiments, the Cas domain is a Cas9 domain. In some embodiments, the Cas domain is a Cas12 domain.

In some embodiments, the Cas9 domain is derived from a bacterial organism such as, Staphylococcus aureus, Streptococcus pyogenes, Neisseria meningitidis, Campylobacter jejuni, Streptococcus pasteurianus, Clostridium cellulolyticum, or Geobacillus thermodenitrificans T1. In some embodiments, the Cas9 domain is derived from Staphylocuccus aureus (SaCas9). In some embodiments, the Cas9 domain is derived from Streptococcus pyogenes (spCas9). In some embodiments, the Cas9 domain is derived from Neisseria meningitidis (NmCas9). In some embodiments, the Cas9 domain is derived from Campylobacter jejuni (CjCas9). In some embodiments, the Cas9 domain is derived from Streptococcus pasteurianus (SpCas9), In some embodiments, the Cas9 domain is derived from Clostridium cellulolyticum(CcCas9). In some embodiments, the Cas9 domain is derived from Geobacillus thermodenitrificans T1 (GtCas9).

In some embodiments, exemplary RNA-guided nuclease Cas9 domains is shown in SEQ ID NO: 710, 711, 712, 713, 714, 715, or 716. In some embodiments, the mutation or amino acid substitution can correspond to any one of SEQ ID NO: 710, 711, 712, 713, 714, 715, or 716.

In some embodiments, the RNA-guided nuclease Staphylococcus aureus Cas9 domain comprises an amino acid sequence that is at least 85%, 90%, 95%, 97%, 98%, or 99% identical, or is identical to any one of SEQ ID NO: 710. In some embodiments, the RNA-guided nuclease Staphylococcus aureus Cas9 domain comprises a mutation or amino acid substitution corresponding to a position selected from any one of D10, H557, N580, H840, D1135, R1335, T1337, T267, L325, V327, D333, A336, 1341, E345, D348, K352, S360, T368, N369, N371, 5372, E373, K386, N393, H408, N410, 1414, A415, T438, Y467, N471, D485, M489, E506, R409, T510, N515, Y518, A539, F550, N551, S596, T602, A611, I617, T620, G654, N667, R685, K695, 1706, K722, A723, K724, M731, F732, K735, S739, P741, E742, E746, Q747, I754, T755, H757, K760, H761, P778, E781, 1783, N784, D785, T786L, L787, Y788, K792, D794, T798, L799, V801, N803, L804, N805, G806, D813, K814, L818, 1819, 5822, E824, L841, G847, D848, Y857, V875, 1876, N884, A888, L890, D894, D895, P897, V903, G920, F924, N929, E936, N937, V941, N942, 5943, C945, E947, K951, L952, 5956, N957, Q958, A959, N974, G975, V983, N984, N985, D986, I991, V993, M995, I996, T999, Y1000, R1001, E1002, L1004, E1005, N1006, M1007, D1009, K1010, R1011, P1012, P1013, I1015, I1016, A1020, S1021, Q1024, K1027, E1039, H1045, 10148, K1050 or a combination thereof. In some embodiments, the RNA-guided nuclease Staphylococcus aureus Cas9 domain comprises a mutation or substitution corresponding to any one or more of D10A, D10E, H557A, N580A, H840A, D1135E, R1335Q, T1337R, T267A, L325F, V327I, D333G, A336S, I341L, E345D, D348N, K352E, S360A, T368A, N369E, N371E, S372P, E373K, K386T, N393R, H408N, N410S, I414M, A415T, T438S, Y467F, N471K, D485E, M489F, E506K, R409K, T510E, N515K, Y518F, A539P, F550Y, N551H, S596A, T602I, A611S, I617V, T620K, G654E, N667D, R685K, K695Q, I706V, K722T, A723T, K724N, M73IT, F732V, K735Q, S739N, P741L, E742G, E746D, Q747D, I754D, T755I, H757R, K760Q, H761S, P778I, E781K, I783V, N784D, D785E, T786L, L787V, Y788H, K792E, D794T, T798R, L799I, V801I, N803S, L804I, N805K, G806N, D813G, K814E, L8181, 1819F, S822P, E824G, L841T, G847S, D848N, Y857H, V8751, 1876V, N884K, A888V, L890R, D894G, D895H, P897L, V903I, G920D, F924L, N929Y, E936D, N937G, V941I, N942D, S943L, C945A, E947K, K951R, L952Q, S956N, N957E, Q958K, A959S, N974D, G975K, V983A, N984S, N985D, D986G, I991V, V993L, M995F, I996V, T999N, Y1000K, R1001E, E1002D, L1004I, E1005K, N1006M, M1007N, D1009L, K1010S, R1011T, P1012S, P1013F, I1015L, I1016R, A1020G, 51021K, Q1024K, K1027S, E1039K, H1045K, I0148M, K1050M or a combination thereof. In certain embodiments, the RNA-guided nuclease Staphylococcus aureus Cas9 domain comprise a mutation or substitution corresponding to D10E substitution. In certain embodiments, the modified I-TevI nuclease domain comprises SEQ ID NO: 700, the linker comprises any one of SEQ ID NOs: 701 or 703 and the RNA-guided nuclease Staphylococcus aureus Cas9 domain comprises any one of SEQ ID NO: 710-715, or 716. In certain embodiments, the modified I-TevI nuclease domain comprises an amino acid sequence that is at least 85%, 90%, 95%, 97%, 98%, or 99% identical, or is identical to any one of SEQ ID NO: 700, the linker domain comprises an amino acid sequence that is at least 85%, 90%, 95%, 97%, 98%, or 99% identical, or is identical to any one SEQ ID NOs: 701 or 703 and the RNA-guided nuclease Staphylococcus aureus Cas9 domain comprises an amino acid sequence that is at least 85%, 90%, 95%, 97%, 98%, or 99% identical to any one SEQ ID NOs: 710-715, or 716.

In some embodiments, the RNA-guided nuclease Staphylococcus aureus Cas9 domain comprises an amino acid sequence that is at least 85%, 90%, 95%, 97%, 98%, or 99% identical to SEQ ID NO: 710. In some embodiments, the RNA-guided nuclease comprises an amino acid sequence having between 85-90%, 90-95%, 95-97%, 97-98%, or 98-99% sequence identity to SEQ ID NO: 710. In some embodiments, the RNA-guided nuclease Staphylococcus aureus Cas9 domain comprises a mutation corresponding to any one of positions D10, H557, N580, H840, D1135, R1335, T1337, or a combination thereof. In some embodiments, the RNA-guided nuclease Staphylococcus aureus Cas9 domain comprises a mutation corresponding to position D10, H557, N580, H840, D1135, R1335, T1337, or a combination thereof. In some embodiments, the RNA-guided nuclease Staphylococcus aureus Cas9 domain comprises a mutation corresponding to any one of D10A, D10E, H557A, N580A, H840A, D1135E, R1335Q, T1337R, or a combination thereof. In some embodiments, the RNA-guided nuclease Staphylococcus aureus Cas9 domain (saCas9) comprises a mutation corresponding to D10E mutation. In some embodiments, the saCas9 comprises a mutation corresponding to a D10E and/or N580A mutation. In some embodiments, the saCas9 comprises a mutation corresponding to a D10A and/or N580A mutation. In some embodiments, the saCas9 comprises a mutation corresponding to a D10E D1135E, R1335Q, and/or T1337R mutation. In some embodiments, the saCas9 comprises a mutation corresponding to D10E, D1135E, R1335Q, T1337R, and/or H840A mutation. In some embodiments, the saCas9 comprises a mutation corresponding to D10E and/or H557A mutation. In some embodiments, the saCas9 comprises a mutation corresponding to a D10E, H840A, D1135E, R1335Q, and/or T1337R mutation.

In some embodiments, the RNA-guide nuclease Cas9 domain is an RNA-guided nuclease Streptococcus pyogenes Cas9 domain. In some embodiments, the RNA-guided nuclease Streptococcus pyogenes Cas9 domain comprises an amino acid sequence as set forth in SEQ ID NO: 711.

In some embodiments, the RNA-guided nuclease Staphylococcus pyogenes Cas9 domain comprises a mutation corresponding to any one of positions D10, S29, F32, D39, R40, H41, S42, I48, C80, S87, K112, H113, K132, K141, D147, L158, E171, P176, I186, V189, Q190, Q194, N199, I201, N202, A203, S204, R205, A210, Q228, L229, G231, S245, T249, S254, D261, T270, N295, T300, D304, V308, N309, I312, T333, A337, E345, F352, Q354, S355, K356, G366, A367, E396, L398, 1414, D428, F429, D435, K468, S469, E470, T472, E480, A486, S490, F498, K500, N501, N504, K528, V530, E532, G533, A538, T555, K570, F575, D605, E611, R629, E634, T638, R655, R664, R671, K705, E706, Q709, K710, S714, G7115, G717, H721, H723, A725, N726, V743, L747, V748, K772, K775, N776, 1788, G792, K797, Y799, T804, N808, L811, R820, N831, R832, V842, L847, N869, E874, N881, Q885, N888, T893, L911, Y945, D946, L949, E952, A1023, Y1036, G1067, G1077, R1078, N1093, R1114, N1115, D1117, A1121, D1125, P1128, K1129, V1146, S1154, S1159, L1164, S1172, N1177, P1178, I1179, D1180, K1211, M1213, G1218, N1234, E1243, K1244, E1253, E1260, K1263, H1264, E1271, Q1272, E1275, V1290, L1291, S1292, A1293, N1295, H1297, R1298, D1299, K1300, R1303, E1307, N1308, I1309, I1310, H1311, L1312, L1315, T1316, N1317, Y1326, D1328, V1342, A1345, I1360, S1363, or a combination thereof. In some embodiments, the RNA-guided nuclease Streptococcus pyogenes Cas9 domain comprises a mutation corresponding to any one of D10E, D10A, S29T, F32M, D39N, R40K, H41Q, S42T, I48L, C80R, S87A, K112D, Hi 13N, K132N, K141E, D147E, L158V, E171Q, P176S, I186K, V189L, Q190H, Q194E, N199R, 1201L, N202E, A203E, S204I, R205K, A210G, Q228A, L229F, G23IN, S245A, T249M, S254A, D261N, T270S, N295K, T300I, D304G, V308A, N309D, I312V, T333A, A337V, E345K, F352S, Q354K, S355T, K356T, G366K, A367T, E396D, L398F, I414V, D428A, F429Y, D435E, K468Q, S469R, E470N, T472A, E480D, A486T, S490L, F498V, K500E, N501H, N504T, K528R, V530I, E532D, G533E, A538E, T555A, K570Q, F575C, D605E, E61 ID, R629K, E634K, T638K, R655H, R664K, R671K, K705V, E706D, Q709K, K710A, S714F, G7115E, G717K, H721K, H723Q, A725S, N726A, V743I, L747I, V748I, K772Q, K775R, N776R, I788M, G792R, K797E, Y799H, T804A, N808D, L811R, R820K, N83ID, R832H, V842I, L847I, N869D, E874A, N881S, Q885R, N888K, T893S, L911A, Y945H, D946G, L949P, E952A, A1023G, Y1036R, G1067E, G1077E, R1078K, N1093T, R1114G, N1115E, D1117A, A1121P, D1125G, P1128T, K1129T, V11461, S1154T, S1159P, L1164V, S1172N, N1177D, P1178S, 11179V, D1180S, K1211R, M1213L, G1218T, N1234H, E1243D, K1244T, E1253K, E1260D, K1263Q, H1264Y, E1271D, Q1272W, E1275H, V1290L, L1291R, S1292A, A1293T, N1295E, H1297N, R1298T, D1299H, K1300L, R1303S, E1307D, N1308S, I1309M, I1310L, H1311N, L1312A, L1315F, T1316S, N1317R, Y1326F, D1328N, V1342I, A1345S, I1360L, S1363N, or a combination thereof. In some embodiments, the RNA-guided nuclease Streptococcus pyogenes Cas9 domain comprises an amino acid sequence having at least 85%, 90%, 95%, 97%, 98%, or 99% sequence identity to SEQ ID NO: 711. In some embodiments, the RNA-guided nuclease Streptococcus pyogenes Cas9 domain comprises an amino acid sequence having between 85-90%, 90-95%, 95-97%, 97-98%, or 98-99% sequence identity to SEQ ID NO: 711.

In some embodiments, the RNA-guide nuclease Cas9 domain is an RNA-guided nuclease Neisseria meningitidis Cas9 domain. In some embodiments, the RNA-guided nuclease Neisseria meningitidis Cas9 domain comprises an amino acid sequence as set forth in SEQ ID NO: 712. Other Neisseria meningitidis Cas9 can be found at www.uniprot.org/uniprot/with accession numbers C9X1G5, A1IQ68, EONB23, A9M1K5, or C6S593. In some embodiments, the RNA-guided nuclease Neisseria meningitidis Cas9 domain comprises a mutation corresponding to any one of positions 19, D16, D30, E31, A94, I103, P124, N164, I213, G229, T241, S376, E393, G454, K471, G490, D660, C665, K764, T770, P803, A841, H842, K843, D844, L846, R847, K854, H855, N856, K858, K862, W865, E868, 1869, A872, D873, N876, Y880, G883, 1886, E887, E890, R895, A898, Y899, G900, G901, N902, A903, K904, Q905, D908, N912, K917, G919, L921, V927, K929, T930, E932, S933, L936, L937, N938, K939, K940, Y943, T944, G949, D950, C958, K965, N966, Q967, F969, A975, E980, N981, I986, D987, C988, K989, G990, Y991, R992, I993, D994, Y997, T998, C1000, S1002, H1004, K1005, Y1006, A1010, F1011, Q1012, K1013, D1014, E1015, K1018, V1019, E1020, F1021, A1022, Y1024, I1025, N1026, C1027, D1028, 51029, 51030, N1031, R1033, F1034, Y1035, L1036, A1037, W1038, K1041, G1042, K1044, E1045, Q1046, Q1047, F1048, R1049, I1050, S1051, T1052, Q1053, N1054, L1055, V1056, L1057, I1058, Y1061, V1063, N1064, or a combination thereof. In some embodiments, the RNA-guided nuclease Neisseria meningitidis Cas9 domain comprises a mutation corresponding to any one of I9M, D16E, D30E, E31K, A94D, I103V, P124C, N164D, I213N, G229D, T241A, S376T, E393K, G454C, K471E, G490C, D660E, C665R, K764E, T770A, P803S, A841Q, H842G, K843H, D844E, L846V, R847K, K854R, H855L, N856D, K858G, K862L, W865P, E868Q, I869L, A872K, D873G, N876K, Y880R, G883E, I886P, E887K, E890E, R895Q, A898T, Y899H, G900K, G901D, N902D, A903P, K904T, Q905K, D908A, N912E, K917Y, G919T, L921Q, V927I, K929Q, T930V, E932K, S933T, L936W, L937V, N938R, K939N, K940H, Y943N, T944G, G949A, D950T, C958E, K965G, N966G, Q967K, F969Y, A975S, E980K, N981G, I986R, D987A, C988V, K989V, G990A, Y991F, R992K, I993D, D994E, Y997F, T998E, C1000R, S1002I, H1004Y, K1005A, Y1006N, A1010K, F1011L, Q1012T, K1013A, D1014K, E1015K, K1018N, V1019E, E1020F, F1021L, A1022G, Y1024F, I1025V, N1026S, C1027L, D1028N, S1029R, S1030A, N1031T, R1033A, F1034I, Y1035D, L1036I, A1037R, W1038T, K1041T, G1042D, K1044T, E1045K, Q1046G, Q1047E, F1048Q, R1049S, I1050V, S1051G, T1052V, Q1053K, N1054T, L1055A, V1056L, L1057S, I1058F, Y1061N, V1063I, N1064D, or a combination thereof. In some embodiments, the RNA-guided nuclease Neisseria meningitidis Cas9 domain comprises an amino acid sequence having at least 85%, 90%, 95%, 97%, 98%, or 99% sequence identity to SEQ ID NO: 712. In some embodiments, the RNA-guided nuclease Neisseria meningitidis Cas9 domain comprises an amino acid sequence having between 85-90%, 90-95%, 95-97%, 97-98%, or 98-99% sequence identity to SEQ ID NO: 712.

In some embodiments, the RNA-guide nuclease Cas9 domain is an RNA-guided nuclease Campylobacter jejuni Cas9 domain. In some embodiments, the RNA-guided nuclease Campylobacterjejuni Cas9 domain comprises an amino acid sequence as set forth in SEQ ID NO: 713. Other Campylobacter jejuni Cas9 can be found at www.uniprot.org/uniprot/with accession numbers Q0P897, A7H5P1, AOA2UOQR81, AOA5Y4VLH1, or AOA381CRM8. In some embodiments, the RNA-guided nuclease Campylobacter jejuni Cas9 domain comprises a mutation corresponding to any one of positions L5, A6, D8, I9, S12, S13, F18, S19, L24, K25, I31, T40, E42, L50, L58, A59, R61, L58, L65, H67AN74, K77, L98, I99, P101, N110, L113, A119, A126, R128, I134, K140, A144, K147, Q151, L156, V184, 5190, F199, D202, G203, R212, F214, K221, E223, Y232, A235, V243, S247, D251, P256, L261, T269, N276, N277, L285, T287, L291, K300, T305, Q308, L312, G314, Y335, K336, 1339, H345, D351, N353, E354, 1362, K370, D383E, 5384, K391, 1396, L403, T405, K413, N419, L421, D430, K432, A437, L453, K457, V462, A465, K472, N477, A492, E495, L525, K526, L527, K531, E532, E542, Q550, E556, H559, Y561, S564, M572, V577, Q581, N587, N596, K600, Q602, K603, Q616, K617, N623, Y624, K633, D634, Y642, N649, D656, L660, D662, K667, V677, E680, K682, L686, H692, T693, V712, I714, V722, K723, S736, L739, K742, L747, N751, F756, R763, Q764, E772, K777, A786, E790, F792, Q800, 5801, G804, L812, E813, V833, 1835, T841, Y845, A855, L856, A863, V864, D879, E883, D900, Q902, K927, F928, V971, T972, or a combination thereof. In some embodiments, the RNA-guided nuclease Campylobacter jejuni Cas9 domain comprises a mutation corresponding to any one of L51, A6G, D8N, D8E, I9L, S12A, S13N, F18L, S19R, L24I, K251, 131V, T40N, E42N, L50E, L58V, A59K, R61K, L58V, L65M, H67A, N74K, K77N, L98T, I99Q, P101I, Ni 10S, L113I, A119S, A126V, R128H, I134S, K140N, A144T, K147E, Q151K, L156M, V184I, S190D, F199L, D202Q, G203E, R212K, F214L, K221K, E223K, Y232F, A235P, V243I, S247I, D251N, P256A, L261S, T269G, N276K, N277S, L285V, T287E, L291I, K300D, T305S, Q308K, L312I, G314N, Y335L, K336N, I339K, H345T, D351I, N353D, E354S, I362T, K370E, D383E, S384K, K391N, I396L, L403Q, T405I, K413R, N419E, L421C, D430E, K432S, A437L, L453I, K457C, V462L, A465D, K472S, N477H, A492K, E495I, L525Q, K526I, L527V, K531E, E532D, E542L, Q550D, E556V, H559Y, Y561R, S564N, M572S, V577T, Q581L, N587G, N596E, K600L, Q602A, K603E, Q616R, K617F, N623F, Y624F, K633T, D634E, Y642W, N649S, D656S, L660I, D662E, K667A, V677Q, E680V, K682S, L686I, H692N, T693F, V712I, I714V, V722I, K723F, S736K, L739F, K742N, L747S, N751L, F756L, R763K, Q764E, E772N, K777H, A786T, E790L, F792P, Q800N, S801T, G804D, L812V, E813K, V833S, I835L, T841K, Y845H, A855S, L856T, A863T, V864P, D879N, E883N, D900G, Q902K, K927N, F928Y, V971L, T972S, or a combination thereof. In some embodiments, the RNA-guided nuclease Campylobacter jejuni Cas9 domain comprises an amino acid sequence having at least 85%, 90%, 95%, 97%, 98%, or 99% sequence identity to SEQ ID NO: 713. In some embodiments, the RNA-guided nuclease Campylobacter jejuni Cas9 domain comprises an amino acid sequence having between 85-90%, 90-95%, 95-97%, 97-98%, or 98-99% sequence identity to SEQ ID NO: 713.

In some embodiments, the RNA-guide nuclease Cas9 domain is an RNA-guided nuclease Streptococcus pasteurianus Cas9 domain. In some embodiments, the RNA-guided nuclease Streptococcus pasteurianus Cas9 domain comprises an amino acid sequence as set forth in SEQ ID NO: 714. Other Streptococcus pasteurianus Cas9 can be found at www.uniprot.org/uniprot/with accession number F5X275.

In some embodiments, the RNA-guided nuclease Streptococcus pasteurianus Cas9 domain comprises a mutation corresponding to any one of positions D11, E85, A88, T92, E96, Y100, T109, D110, D113, E115, R116, D125, I127, K128, E132, S147, I185, A187, K228, Y229, T232, M255, S271, N273, A294, A327, E355, K357, N379, T380, S382, A385, D439, R440, S464, H469, Y519, I528, N569, I581, A607, K632, D633, H635, E636, A647, D648, T703, P705, K712, S713, A724, V750, D882, S951, D977, E979, S1014, H1027, I1030, E1081, D1082, D1086, K1088, S1089, N1090, R1092, T1093, I1094, C1095, A1138, Y1139, D1141, T1142, F1158, A1168, E1190, E1198, H1202, I1204, R1205, I1210, K1224, S1232, M1240, V1241, I1242, P1243, G1424, K1248, Q1254, N1257, S1258, T1262, K1263, Y1264, D1266, A1270, K1277, D1284, L1288, V1302, N1316, T1346, I1374,or a combination thereof. In some embodiments, the RNA-guided nuclease Streptococcus pasteurianus Cas9 domain comprises a mutation corresponding to any one of D11E, D11A, E85D, A88T, T92A, E96D, Y100Q, T109D, D110N, D113N, E115D, R116S, D125E, I127D, K128A, E132K, S147T, I185L, A187T, K228N, Y229N, T232K, M255T, S271T, N273E, A294S, A327V, E355K, K357Q, N379G, T380I, S382T, A385N, D439E, R440E, S464A, H469R, Y519F, I528V, N569D, I581V, A607S, K632R, D633E, H635Q, E636Q, A647K, D648Q, T703A, P705S, K712E, S713A, A724T, V750I, D882G, S951R, D977E, E979K, S1014P, H1027R, I1030V, E1081G, D1082E, D1086N, K1088R, S1089T, N1090D, R1092E, T1093K, I1094V, C1095R, A1138V, Y1139L, D1141E, T1142P, F1158L, A1168T, E1190K, E1198K, H1202Q, I1204V, R1205Q, I1210M, K1224R, S1232T, M1240I, V1241M, I1242L, P1243S, G1424A, K1248A, Q1254H, N1257G, S1258N, T1262A, K1263E, Y1264H, D1266K, A1270E, K1277E, D1284N, L1288V, V1302A, N1316D, T1346N, I1374L,or a combination thereof. In some embodiments, the RNA-guided nuclease Streptococcus pasteurianus Cas9 domain comprises an amino acid sequence having at least 85%, 90%, 95%, 97%, 98%, or 99% sequence identity to SEQ ID NO: 714. In some embodiments, the RNA-guided nuclease Streptococcus pasteurianus Cas9 domain comprises an amino acid sequence having between 85-90%, 90-95%, 95-97%, 97-98%, or 98-99% sequence identity to SEQ ID NO: 714.

In some embodiments, the RNA-guide nuclease Cas9 domain is an RNA-guided nuclease Clostridium cellulolyticum Cas9 domain. In some embodiments, the RNA-guided nuclease Clostridium cellulolyticum Cas9 domain comprises an amino acid sequence as set forth in SEQ ID NO: 715. Other Clostridium cellulolyticum Cas9 can be found at www.uniprot.org/uniprot/with accession number B8I085.

In some embodiments, the RNA-guided nuclease Clostridium cellulolyticum Cas9 domain comprises a mutation corresponding to any one of positions T4, D10, V9, D20, K21, 127, C33, K36, A47, A49, S64, Q65, E102, L103, T122, I1124, K131, D137, R163, G166, I1169, F170, V183, D184, I187, E193, K200, K208, L209, D221, N224, E227, F228, S234, V242, K244, L252, T256, C258, S261, V413, M415, K416, R417, K424, Y426, K427, S429, D430, A468, T470, A472, A478, Q481, K482, L485, A497, L535, W540, R541, E544, G554, P556, I1570, Y574, M580, Y584, M585, T592, D593, V606, W607, I647, N650, S693, L697, E702, S704, A713, V714, I1715, D776, L847, G850, G853, A854, R860, I900, H904, M905, I906, E921, Q923, S929, T930, H931, Q939, N994, I997, N1000, K1001, S1002, I1003, K1005, P1008, or a combination thereof. In some embodiments, the RNA-guided nuclease Clostridium cellulolyticum Cas9 domain comprises a mutation corresponding to any one of T4S, D10E, V9I, D20N, K21E, I27E, C33I, K36V, A47S, A49P, S64R, Q65H, E102L, L103V, T122V, I124F, K131Q, D137E, R163Q, G166S, I169L, F170L, V183G, D184G, I187T, E193S, K200Q, K208A, L209Y, D221K, N224Q, E227S, F228S, S234T, V242I, K244N, L252K, T256K, C258T, S261F, V413K, M415L, K416R, R417N, K424Q, Y426I, K427P, S429H, D430Q, A468S, T470S, A472V, A478G, Q481K, K482R, L485S, A497M, L535H, W540Y, R541K, E544Q, G554F, P556S, I570V, Y574I, M580F, Y584N, M585N, T592A, D593A, V606W, W607F, I647R, N650H, S693K, L697F, E702Q, S704N, A713V, V7141, I1715V, D776E, L847A, G850P, G853A, A854P, R860K, I900V, H904D, M905V, I906L, E921Y, Q923E, S929D, T930E, H931Y, Q939P, N994Q, I997P, N1000R, K1001M, S1002N, I1003K, K1005H, P1008K, or a combination thereof. In some embodiments, the RNA-guided nuclease Clostridium cellulolyticum Cas9 domain comprises an amino acid sequence having at least 85%, 90%, 95%, 97%, 98%, or 99% sequence identity to SEQ ID NO: 715. In some embodiments, the RNA-guided nuclease Clostridium cellulolyticum Cas9 domain comprises an amino acid sequence having between 85-90%, 90-95%, 95-97%, 97-98%, or 98-99% sequence identity to SEQ ID NO: 715.

In some embodiments, the RNA-guide nuclease Cas9 domain is an RNA-guided nuclease Geobacillus thermodenitrificans T1 Cas9 domain. In some embodiments, the RNA-guided nuclease Geobacillus thermodenitrificans T1 Cas9 domain comprises an amino acid sequence as set forth in SEQ ID NO: 716. Other Geobacillus thermodenitrificans T1 Cas9 can be found at www.uniprot.org/uniprot/ with accession number A0A1W6VMQ3.

In some embodiments, the RNA-guided nuclease Geobacillus thermodenitrificans T1 Cas9 domain comprises a mutation corresponding to any one of positions K2, D8, I14, D35, K41, F74, V75, K91, I117, R128, T136, Q151, S152, S156, A161, V164, S171, E178, D179, V185, R192, K195, A199, Y204, 1207, V208, A212, H215, S219, F227, T260, V261, V271, G274, 1276, A278, L279, D282, 1287, K289, H293, F299, V302, N307, R313, L317, L318, V331, G337, K341, S348, A354, A355, K356, R359, M372, T377, R380, E395, D399, E404, S416, T441, R445, N464, E504, S508, M515, Q516, E520, G521, V534, L545, K559, T578, K603, T612, L619, S621, N656, N660, L673, D685, I699, N708, N717, R737, V738, S752, D756, Q771, N777, N792, E793, 1811, 1824, K839, Q845, K848, T849, L895, I902, T908, V929, I943, I946, M948, F990, T995, V1000, Q1014, D1017, S1019, N1020, G1021, S1024, N1030, N1031, R1035, S1036, I1037, V1067, S1071, A1075, I1079, or a combination thereof. In some embodiments, the RNA-guided nuclease Geobacillus thermodenitrificans T1 Cas9 domain comprises a mutation corresponding to any one of positions D8, D179, D282, D399, D685, D756, D1071. In some embodiments, the RNA-guided nuclease Geobacillus thermodenitrificans T1 Cas9 domain comprises a mutation corresponding to any one of K2R, D8E, D8A, I14V, D35E, K41Q, F74V, V75I, K91E, 1117V, R128K, T136S, Q151R, S152A, S156G, A161G, V164I, S171A, E178G, D179E, V185I, R192H, K195R, A199S, Y204F, I207M, V208S, A212K, H215N, S219T, F227V, T260I, V261A, V271I, G274S, I276A, A278G, L279P, D282E, I287L, K289E, H293Q, F299Y, V302I, N307R, R313Y, L317I, L318V, V3311, G337D, K341Q, S348K, A354K, A355S, K356S, R359L, M372L, T377A, R380H, E395P, D399N, E404N, S416T, T441S, R445K, N464T, E504D, S508T, M515T, Q516K, E520D, G521E, V534M, L545H, K559R, T578V, K603R, T612I, L619V, S621T, N656M, N660S, L673F, D685E, I699V, N708E, N717D, R737K, V738I, S752A, D756E, Q771R, N777H, N792D, E793Q, 1811V, I824V, K839T, Q845K, K848A, T849S, L895P, I902V, T908K, V929V, I943V, I946M, M948I, F990L, T995I, V1000G, Q1014K, D1017H, 51019G, N1020T, G1021A, S1024E, N1030C, N1031S, R1035S, S1036G, I1037V, V1067L, S1071A, A1075T, I1079V, or combination thereof. In some embodiments, the RNA-guided nuclease Geobacillus thermodenitrificans T1 Cas9 domain comprises a mutation corresponding to any one of positions D8E, D179E, D282E, D399N, D685E, D756E, D1071H. In some embodiments, the RNA-guided nuclease Geobacillus thermodenitrificans T1 Cas9 domain comprises an amino acid sequence having at least 85%, 90%, 95%, 97%, 98%, or 99% sequence identity to SEQ ID NO: 716. In some embodiments, the RNA-guided nuclease Geobacillus thermodenitrificans T1 Cas9 domain comprises an amino acid sequence having between 85-90%, 90-95%, 95-97%, 97-98%, or 98-99% sequence identity to SEQ ID NO: 716.

TABLE 2
Exemplary Cas9 domains and PAM Motifs

| Cas Domain | PAM Motif |
|---|---|
| NmCas9 from | 5′-NNNNGMTT-3′ |
| Neisseria | (M = A or C) |
| meningitidis | (SEQ ID NO: 870) |
| SpCas9 from | 5′-NRG-3′ |
| Streptococcus | (R = A or G) |
| pyogenes | (SEQ ID NO: 871) |
| StCas9 from | 5′-NNAGAAW-3′ |
| Streptococcus | (W = A or T) |
| thermophilus | (SEQ ID NO: 872) |
| CjCas9 from | 5′-NNNNRYAC-3′ |
| Campylobacter | (R = A or G, Y = C or T) |
| jejuni | (SEQ ID NO: 873) |
| SpCas9 from | 5′-NNGTGA-3′ |
| Streptococcus | (SEQ ID NO: 874) |
| pasteurianus | |
| Nme2Cas9 from | 5′-NNNNCC-3′ |
| Neisseria | (SEQ ID NO: 875) |
| meningitidis | |
| CcCas9 from | 5′-NNNNGNA-3′ |
| Clostridium | (SEQ ID NO: 876) |
| cellulolyticum | |
| ThermoCas9 from | 5′-NNNNCNR-3′ |
| Geobacillus | (SEQ ID NO: 877) |
| thermodenitrificansT1 | |

In some embodiments, the Cas domain is CasX domain. In some embodiments, the CasX domain is derived from Planctomycetes bacterium. In some embodiments, the CasX domain is from Deltaproteobacteria. In some embodiments, the CasX domain comprises an amino acid sequence at least about 85%, 90%, 95%, 97%, 98%, or 99% identical, or is identical to SEQ ID NO: 721. In some embodiments, the CasX domain comprises a mutation corresponding to any one of positions R11, R12, V14, K15, S17, N18, A22, G23, T25, P38, K41, E42, N46, L47, N53, I54, P57, T61, S62, R63, A64, E75, H82, Q89, P104, N106, I113, N199, S124, S125, C133, Y137, N145, D146, H151, 5161, R165, N177, L180, R202, N205, G215, C219, V236, T241, L248, 1254, S269, 1290, E291, V297, Q299, 1314, E318, Q323, L333, E359, D360, K362, Q366, N367, L368, A369, G370, Y371, H404, H409, G410, E411, Y417, V428, E429, S432, K433, L437, S443, A451, 1464, A470, 1502, L503, 1531, G537, L540, N553, 1559, S563, V571, N579, H589, S607, L608, L620, R623, R624, L644, S646, M652, I657, R679, L684, N686, H689, S696, T702, T737, L742, Y744, Q748, M751, 1753, A771, R777, P792, 5818, R823, V824, E826, K827, A832, T833, M836, 1839, G841, V846, N860, V862, D864, V867, V877, S883, S889, G890, S894, K908, N913, F916, T918, R936, Q938, Y940, K942, S963, R966, K967, K968, or any combination thereof. In some embodiments, the CasX domain comprises a mutation or substitution corresponding to any one or more of R11K, R12K, V14S, K15A, S17N, N18A, A22V, G23S, T25S, P38D, K41K, E42K, N46K, L47R, N53V, I54M, P57V, T61N, S62A, R63A, A64N, E75K, H82Q, Q89K, P104S, N106K, 1113K, N199K, S124T, S125A, C133G, Y137F, N145S, D146E, H151Y, S161A, R165K, N177S, L180A, R202K, N205T, G215A, C219Y, V236I, T241S, L248I, I254V, S269G, I290V, E291D, V297I, Q299R, I314L, E318D, Q323L, L333V, E359D, D360M, K362R, Q366S, N367G, L368V, A369T, G370A, Y371E, H404Y, H409Y, G410A, E411G, Y417F, V428I, E429A, S432T, K433S, L437R, S443A, A451V, I464L, A470M, I502V, L503V, I531L, G537K, L540I, N553S, I559L, S563G, V571L, N579Q, H589T, S607L, L608I, L620I, R623K, R624K, L644V, S646P, M652V, I657V, R679E, L684S, N686G, H689D, S696G, T702A, T737S, L742F, Y744H, Q748H, M751V, I753V, A771T, R777K, P792T, S818T, R823G, V824M, E826V, K827R, A832S, T833D, M836A, I839L, G841N, V846A, N860T, V862E, D864E, V867A, V877G, S883K, S889R, G890D, S894F, K908Q, N913D, F916H, T918V, R936N, Q938N, Y940F, K942S, S963A, R966K, K967R, K968R, or a combination thereof.

In some embodiments, the Cas12 domain is from Acidaminococcus sp. BV3L6. In some embodiments, the Cas12 domain comprises an amino acid sequence at least about 85%, 90%, 95%, 97%, 98%, or 99% identical, or is identical to SEQ ID NO: 720. In some embodiments, the Cas12 domain comprises a mutation corresponding to any one of positions T1, Q2, E4, G5, N8, L9, K28, H29, 130, Q31, E32, Q33, F35, 136, E37, E38, A41, N43, D44, H45, E48, 152, R55, T59, Y60, A61, D62, Q63, C64, Q66, L67, Q69, L70, N74, S76, A77, D80, S81, Y82, E85, E88, T90, R91, N92, A93, I95, E97, A99, T100, Y101, N103, A104, H106, D107, I110, R112, T113, D114, R159, S169, S185, A187, I192, D195, K201, T212, R218, N223, 1228, S233, 1236, E237, V239, F242, Q249, Y257, V279, 1284, F305, N313, S324, 1329, S331, T337, L338, L345, E349, S357, 1358, N386, 1393, L396, 1400, S403, V408, Q409, G427, K428, Q436, L442, S468, Q469, S472, L473, L479, E487, S488, A497, L510, A516, K522, Q535, M536, S541, V545, K549, N550, G552, V557, N559, S586, Y596, A601, I604, A613, S628, E637, A657, K660, G663, Q665, C673, L683, L697, A711, L717, Q723, A733, E735, Y740, K751, K756, G766, 1778, R793, L844, 1858, S865, 1874, H898, I903, I916, L931, K941, N945, V951, S958, V959, D965, I938, H984, A1009, C1024, G1037, T1049, G1055, T1056, Y1068, L1075, V1083, K1085, L1097, H1104, D1106, D1111, L1122, A1134, V1138, D1147, V1160, P1161, R1171, R1173, Y1176, N1205, D1207, S1220, V1221, A1230, N1237, L1243, M1259, Q1274, G1291, Q1295, A1299, or L1304. In some embodiments, the Cas12 domain comprises a mutation or substitution corresponding to any one or more of TlS, Q2N, E4S, G5E, N8H, L9K, K28E, H29N, I30L, Q31T, E32A, Q33Y, F35M, I36V, E37N, E38D, A41L, N43S, D44E, H45N, E48K, I52V, R55K, T59Y, Y60F, A61I, D62E, Q63E, C64T, Q66K, L67H, Q69A, L70I, N74P, S76Y, A77K, D80T, S81A, Y82F, E85D, E88L, T90N, R91N, N92T, A93N, I95R, E971, A99D, T100N, Y101C, N103K, A104S, H106A, D107G, I110E, R112K, T113V, D114P, R159K, S169V, S185A, A187S, I192L, D195E, K201I, T212K, R218N, N223T, I228T, S233G, I236L, E237D, V239I, F242V, Q249C, Y257F, V279T, I284V, F305Y, N313S, S324N, I329L, S331A, T337E, L338K, L345I, E349Q, S357L, I358A, N386D, I393V, L396A, I400L, S403N, V408I, Q409E, G427D, K428D, Q436A, L442I, S468V, Q469L, S472A, L473V, L479T, E487D, S488D, A497V, L510I, A516V, K522Q, Q535S, M536N, S541D, V545E, K549Q, N550Q, G552C, V557E, N559E, S586N, Y596Q, A601S, I604L, A613D, S628N, E637T, A657D, K660R, G663N, Q665K, C673H, L683V, L697V, A711G, L717F, Q723E, A733L, E735D, Y740F, K751E, K756A, G766A, I778V, R793P, L844F, I858V, S865T, I874L, H898N, I903V, I916A, L931F, K941N, N945Q, V951I, S958T, V959A, D965E, I938V, H984Q, A1009S, C1024Y, G1037S, T1049E, G1055R, T1056N, Y1068F, L1075A, V1083R, K1085G, L10971, H1104K, Di106N, D1111N, L1122K, A1134D, V11381, D1147A, V1160E, P1161F, R1171Q, R1173E, Y1176L, N1205T, D1207N, S1220L, V1221T, A1230E, N1237S, L1243I, M1259K, Q1274L, G1291A, Q1295N, A1299N, or L1304K.

In some embodiments, the chimeric nuclease comprises anon-naturally occurring Cas domain. In some embodiments, the chimeric nuclease may comprise a Cas domain from other Class 1 or Class 2 CRISPR-Cas proteins, CRISPR-Cas3, CRISPR-Cascade, or Cas13d.

### Nuclear Localization Signals

In some embodiments, a chimeric nuclease further comprises one or more nuclear localization sequences (NLS). In some embodiments, the NLS helps promote translocation of a protein into the cell nucleus. In some embodiments, a chimeric nuclease comprises one or more NLSs. In some embodiments, the chimeric nuclease is fused to or linked to one or more NLSs

In some embodiments, a chimeric nuclease comprises at least one NLS. In some embodiments, a chimeric nuclease comprises at least two NLSs. In embodiments with at least two NLSs, the NLSs can be the same NLS, or they can be different NLSs.

In some embodiments, a chimeric nuclease may further comprise at least one nuclear localization sequence (NLS). In some embodiments, a chimeric nuclease may further comprise 1 NLS. In some cases, a chimeric nuclease may further comprise 2 NLSs. In some embodiments, a chimeric nuclease may further comprise 3 NLSs. In some embodiments, a chimeric nuclease can further comprise at least 4 NLSs.In some embodiments, a chimeric nuclease can further comprise at least 5 NLSs In some embodiments, a chimeric nuclease can further comprise at least 6 NLSs In some embodiments, a chimeric nuclease can further comprise at least 7 NLSs In some embodiments, a chimeric nuclease can further comprise at least 8 NLSs In some embodiments, a chimeric nuclease can further comprise at least 9 NLSs In some embodiments, a chimeric nuclease can further comprise no more than 10 NLSs.

In addition, the NLSs can be expressed as part of a chimeric nuclease. In some embodiments, a NLS can be positioned almost anywhere in a protein's amino acid sequence, and generally comprises a short sequence of three or more or four or more amino acids. The location of the NLS fusion can be at the N-terminus, the C-terminus, or positioned anywhere within a sequence of a chimeric nuclease or a component thereof (e.g., inserted between the I-TevI domain and Cas domain of a chimeric nuclease, between the I-TevI domain and a linker domain, between a Cas domain polymerase and a linker domain or at the N-terminus or the C-terminus of the chimeric nuclease). In some embodiments, a chimeric nuclease comprises an NLS at the N terminus. In some embodiments, a chimeric nuclease comprises an NLS at the C terminus. In some embodiments, a chimeric nuclease comprises at least one NLS at both the N terminus and the C terminus. In some embodiments, a chimeric nuclease comprises two NLSs at the N terminus and/or the C terminus.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a chimeric nuclease comprise bipartite NLSs. In some embodiments, a nuclear localization signal (NLS) is predominantly basic. In some embodiments, the one or more NLSs of a chimeric nuclease are rich in lysine and arginine residues. In some embodiments, the one or more NLSs of a chimeric nuclease comprise proline residues. In some embodiments, a nuclear localization signal (NLS) comprises the sequence MDSLLMNRRKFLYQFKNVRWAKGRRETYLC (SEQ ID NO: 742), KRTADGSEFESPKKKRKV (SEQ ID NO: 743), KRTADGSEFEPKKKRKV (SEQ ID NO: 744), NLSKRPAAIKKAGQAKKKK (SEQ ID NO: 745), RQRRNELKRSF (SEQ ID NO: 746), or NQSSNFGPMKGGNFGGRSSGPYGGGGQYFAKPRNQGGY (SEQ ID NO: 747).

In some embodiments, a NLS is a monopartite NLS. For example, in some embodiments, a NLS is a SV40 large T antigen NLS PKKKRKV (SEQ ID NO: 740). In some embodiments, a NLS is a bipartite NLS. In some embodiments, a bipartite NLS comprises two basic domains separated by a spacer sequence comprising a variable number of amino acids. In some embodiments, a NLS is a bipartite NLS. In some embodiments, a bipartite NLS consists of two basic domains separated by a spacer sequence comprising a variable number of amino acids. In some embodiments, the spacer amino acid sequence comprises the sequence KRXXXXXXXXXXKKKL (Xenopus nucleoplasmin NLS) (SEQ ID NO: 748), wherein X is any amino acid. In some embodiments, the NLS comprises a nucleoplasmin NLS sequence KRPAATKKAGQAKKKK (SEQ ID NO: 741). In some embodiments, a NLS is a noncanonical sequences such as M9 of the hnRNP Al protein, the influenza virus nucleoprotein NLS, and the yeast Gal4 protein NLS. In some embodiments, a NLS is a noncanonical sequences such as M9 of the hnRNP Al protein, the influenza virus nucleoprotein NLS, and the yeast Gal4 protein NLS.

In certain embodiments, said chimeric nuclease comprises a nuclear localization signal. In certain embodiments, the nuclear localization signal comprises an SV40 nuclear localization signal comprising the amino acid sequence SEQ ID NO: 740 (PKKKRKV). In certain embodiments, the nuclear localization signal comprises a Nucleoplasmin nuclear localization signal comprising the amino acid sequence SEQ ID NO: 741 (KRPAATKKAGQAKKKK).

Non-limiting examples of NLS sequences are provided in Table 3 below.

TABLE 3
Exemplary nuclear localization sequences

| | | SEQ |
|---|---|---|
| | | ID |
| Description | Sequence | NO: |
| NLS of SV40 Large | PKKKRKV | 880 |
| T-AG | | |
| NLS | MKRTADGSEFESPKKKRKV | 881 |
| NLS | MDSLLMNRRKFLYQFKNVR | 882 |
| | WAKGRRETYLC | |
| NLS of Nucleoplasmin | AVKRPAATKKAGQAKKKKLD | 883 |
| NLS of EGL-13 | MSRRRKANPTKLSENAKKLA | 884 |
| | KEVEN | |
| NLS of C-Myc | PAAKRVKLD | 885 |
| NLS of Tus-protein | KLKIKRPVK | 886 |
| NLS of polyoma large | VSRKRPRP | 887 |
| T-AG | | |
| NLS of Hepatitis D | EGAPPAKRAR | 888 |
| virus antigen | | |
| NLS of murine p53 | PPQPKKKPLDGE | 889 |
| NLS of PE1 and PE2 | SGGSKRTADGSEFEPKKKRKV | 890 |

### Donor Polynucleotide

The chimeric nuclease of the present disclosure may be used or administered with a donor nucleic acid sequence. A nucleic acid sequence is a sequence that can be used as a template to replace a sequence excised by the chimeric nuclease. In some embodiments, the donor nucleic acid restores a non-oncogenic function of a gene comprising the oncogenic mutation (i.e., restoring wild-type function). In some embodiments, the donor nucleic acid is DNA.

The methods and techniques described herein are useful for the genetic modification of a cell having an oncogene of a population of cells. The donor nucleic acid may be configured for incorporation by homologous recombination. Such donor DNAs for incorporation by homologous recombination may comprise a first flanking homology region, an exogenous polynucleotide sequence of interest, and a second flanking homology region. Alternatively, the exogenous donor DNA may be inserted into a genomic location by incorporation into a genomic location at a single double strand break or a dual double stranded break with the aid of non-homologous end joining.

When a chimeric nuclease (e.g., TevSaCas9) cleaves a double stranded DNA, the Cas (e.g., Cas9) can leave a blunt end and the I-TevI can leave a 3′ overhang. Donor DNAs supplied may include a blunt end and a nucleotide 3′ overhang configured to bind the created 3′ overhang in the chimeric nuclease (TevCas9) cleaved site. In some embodiments, the donor nucleic acid comprises a blunt end and a 3′ overhang. In some embodiments, the 3′ overhang is at least 1, 2, 3, 4, or 5 nucleotides in length. It would be understood that the length of the nucleotide overhand may be altered to accommodate the use of a Cas domain that generates an overhand not equal to two nucleotides.

In some embodiments, the donor DNA comprises DNA sequences that are intended to be inserted into a site of a gene, such as an oncogene. In certain embodiments, the donor DNA comprises double-stranded DNA of the same length cleaved by the nuclease and also comprising complimentary DNA ends to those cleaved by the chimeric nuclease. In certain embodiments, the donor DNA comprises 5′ ends of the DNA that are phosphorylated. In certain embodiments, the donor DNA comprises circular double-strand DNA comprising an I-TevI target site and Cas target site where the product cleaved from the double-strand DNA contains complimentary ends to those cleaved by the chimeric nuclease.

The donor nucleic acid may comprise homology arms that flank either end of the DNA sequences to be inserted into a gene. In some embodiments, the homology arms comprise a 5′ and 3′ homology arm that flank both ends of the DNA sequences to be inserted. In some embodiments, the 5′ and 3′ homology arms are identical or different in length.

The double-stranded donor nucleic acid may contain different 5′-end chemical modifications such as biotin. Other versions of the donor DNA might include stability modifications to the 2′ position of the ribose, including but not limited to 2′-fluoro, 2′-amino, and 2′-O-methyl. In some embodiments, the donor nucleic acid may contain 3′-end modifications such as an inverted dT or biotin. Other versions of the donor nucleic acid might include locked nucleic acids (LNAs) in which the 2′-O and 4′-C atoms of the ribose sugar are joined through a methylene bridge. Other versions of the double-stranded donor nucleic acid might include circular plasmid DNA containing a chimeric nuclease target site in which cleavage with the chimeric nuclease creates complimentary DNA ends to those in the genome target. The double stranded donor nucleic acid may comprise a synthetic or amplified linear double stranded DNA. In certain embodiments the donor nucleic acid is supplied using a viral vector such as an adeno-associated virus or lentivirus.

### Guide RNA

The chimeric nuclease of the present disclosure can further comprise a guide RNA. A guide RNA might target the same region of DNA in the oncogenes but contain different sequences to account for genetic polymorphism in populations. Other versions of the guide RNA might target different oncogenes. Other versions might contain a mixture of guide RNAs to target multiple sequences within the same gene. Guide RNAs may comprise a single strand comprising all necessary elements for activity (e.g., target binding and nuclease binding). Alternatively guide RNAs may comprise two or more non-covalently bound nucleic acids that forma single moiety due to base paring between the two or more nucleic acids.

gRNAs are generally supported by a scaffold, wherein a scaffold refers to the portions of gRNA or crRNA molecules comprising sequences which are substantially identical or are highly conserved across natural biological species (e.g., not conferring target specificity). Scaffolds include the tracrRNA segment and the portion of the crRNA segment other than the polynucleotide-targeting guide sequence at or near the 5′ end of the crRNA segment, excluding any unnatural portions comprising sequences not conserved in native crRNAs and tracrRNAs. In some embodiments, the gRNA comprises a CRISPR RNA (crRNA):trans activating cRNA (tracrRNA) duplex. In some embodiments, the gRNA comprises a stem-loop that mimics the natural duplex between the crRNA and tracrRNA. In some embodiments, the stem-loop comprises a nucleotide sequence comprising non-naturally occurring sequence. For example, in some embodiments, the composition comprises a synthetic or chimeric guide RNA comprising a crRNA, stem, and tracrRNA.

Generally, a protospacer adjacent motif (PAM) is also an important sequence element mediating enzymatic activity of a Cas nuclease. A PAM sequence or element also refers to and includes an approximately 2-6 base pair DNA sequence that is an important targeting component of a Cas nuclease. The PAM sequence further comprises, in certain instances, a DNA sequence that may be required for a Cas/sgRNA to form an R-loop to interrogate a specific DNA sequence through Watson-Crick pairing of its guide RNA with the genome. In certain instances, the PAM specificity can be a function of the DNA-binding specificity of the Cas protein (e.g., a PAM recognition domain of a Cas), wherein, a protospacer adjacent motif recognition domain refers to a Cas amino acid sequence that comprises a binding site to a DNA target PAM sequence.

Typically, the PAM sequence is on either strand, and is downstream in the 5′ to 3′ direction of Cas9 cut site. The canonical PAM sequence (i.e., the PAM sequence that is associated with the Cas9 nuclease of Streptococcus pyogenes or SpCas9) is 5′-NGG-3′ (SEQ ID NO: 760) wherein “N” is any nucleobase followed by two guanine (“G”) nucleobases. Different PAM sequences can be associated with different Cas9 nucleases or equivalent proteins from different organisms. In addition, any given Cas9 nuclease, e.g., SpCas9, may be modified to alter the PAM specificity of the nuclease such that the nuclease recognizes alternative PAM sequence. In the CRISPR-Cas system derived from S. pyogenes (spCas9), the protospacer region DNA typically immediately precedes a 5′-NGG (SEQ ID NO: 761) or NAG (SEQ ID NO: 762) proto-spacer adjacent motif (PAM). Other Cas9 orthologs can have different PAM specificities. For example, Cas9 from S. thermophilus (stCas9) requires 5′-NNAGAA (SEQ ID NO: 763) for CRISPR 1 and 5′-NGGNG (SEQ ID NO: 764) for CRISPR3 and Neiseria menigiditis (nmCas9) requires 5′-NNNNGATT (SEQ ID NO: 765). Cas9 from Staphylococcus aureus subsp. aureus (saCas9) requires 5′-NNGRRT (SEQ ID NO: 766) (R=A or G). In some embodiments, Cas9 enzymes from different bacterial species (i.e., Cas9 orthologs) can have varying PAM specificities. For example, Cas9 from Staphylococcus aureus (SaCas9) recognizes NGRRT (SEQ ID NO: 767) or NGRRN (SEQ ID NO: 768). In addition, Cas9 from Neisseria meningitis (NmCas) recognizes NNNNGATT (SEQ ID NO: 769). In another example, Cas9 from Streptococcus thermophilis (StCas9) recognizes NNAGAAW (SEQ ID NO: 770). In still another example, Cas9 from Treponema denticola (TdCas) recognizes NAAAAC (SEQ ID NO: 771). These are example are not meant to be limiting. It will be further appreciated that non-SpCas9s bind a variety of PAM sequences, which makes them useful when no suitable SpCas9 PAM sequence is present at the desired target cut site. Furthermore, non-SpCas9s can have other characteristics that make them more useful than SpCas9.

In some embodiments, the gRNA spacer sequence comprises about 15 nucleotides to about 28 nucleotides. In some embodiments, the gRNA comprises at least about 15 nucleotides. In some embodiments, the gRNA spacer sequence comprises at most about 28 nucleotides. In some embodiments, the gRNA spacer sequence comprises about 15 nucleotides to about 16 nucleotides, about 15 nucleotides to about 17 nucleotides, about 15 nucleotides to about 18 nucleotides, about 15 nucleotides to about 19 nucleotides, about 15 nucleotides to about 20 nucleotides, about 15 nucleotides to about 21 nucleotides, about 15 nucleotides to about 22 nucleotides, about 15 nucleotides to about 23 nucleotides, about 15 nucleotides to about 24 nucleotides, about 15 nucleotides to about 25 nucleotides, about 15 nucleotides to about 28 nucleotides, about 16 nucleotides to about 17 nucleotides, about 16 nucleotides to about 18 nucleotides, about 16 nucleotides to about 19 nucleotides, about 16 nucleotides to about 20 nucleotides, about 16 nucleotides to about 21 nucleotides, about 16 nucleotides to about 22 nucleotides, about 16 nucleotides to about 23 nucleotides, about 16 nucleotides to about 24 nucleotides, about 16 nucleotides to about 25 nucleotides, about 16 nucleotides to about 28 nucleotides, about 17 nucleotides to about 18 nucleotides, about 17 nucleotides to about 19 nucleotides, about 17 nucleotides to about 20 nucleotides, about 17 nucleotides to about 21 nucleotides, about 17 nucleotides to about 22 nucleotides, about 17 nucleotides to about 23 nucleotides, about 17 nucleotides to about 24 nucleotides, about 17 nucleotides to about 25 nucleotides, about 17 nucleotides to about 28 nucleotides, about 18 nucleotides to about 19 nucleotides, about 18 nucleotides to about 20 nucleotides, about 18 nucleotides to about 21 nucleotides, about 18 nucleotides to about 22 nucleotides, about 18 nucleotides to about 23 nucleotides, about 18 nucleotides to about 24 nucleotides, about 18 nucleotides to about 25 nucleotides, about 18 nucleotides to about 28 nucleotides, about 19 nucleotides to about 20 nucleotides, about 19 nucleotides to about 21 nucleotides, about 19 nucleotides to about 22 nucleotides, about 19 nucleotides to about 23 nucleotides, about 19 nucleotides to about 24 nucleotides, about 19 nucleotides to about 25 nucleotides, about 19 nucleotides to about 28 nucleotides, about 20 nucleotides to about 21 nucleotides, about 20 nucleotides to about 22 nucleotides, about 20 nucleotides to about 23 nucleotides, about 20 nucleotides to about 24 nucleotides, about 20 nucleotides to about 25 nucleotides, about 20 nucleotides to about 28 nucleotides, about 21 nucleotides to about 22 nucleotides, about 21 nucleotides to about 23 nucleotides, about 21 nucleotides to about 24 nucleotides, about 21 nucleotides to about 25 nucleotides, about 21 nucleotides to about 28 nucleotides, about 22 nucleotides to about 23 nucleotides, about 22 nucleotides to about 24 nucleotides, about 22 nucleotides to about 25 nucleotides, about 22 nucleotides to about 28 nucleotides, about 23 nucleotides to about 24 nucleotides, about 23 nucleotides to about 25 nucleotides, about 23 nucleotides to about 28 nucleotides, about 24 nucleotides to about 25 nucleotides, about 24 nucleotides to about 28 nucleotides, or about 25 nucleotides to about 28 nucleotides. In some embodiments, the gRNA spacer sequence comprises about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, or about 28 nucleotides.

In some embodiments, the guide RNA comprises different nucleobases for stability including, but not limited to, a 5-methylcytosine; a 5-hydroxymethyl cytosine; a xanthine; a hypoxanthine; a 2-aminoadenine; a 6-methyl derivative of adenine; a 6-methyl derivative of guanine; a 2-propyl derivative of adenine; a 2-propyl derivative of guanine; a 2-thiouracil; a 2-thiothymine; a 2-thiocytosine; a 5-propynyl uracil; a 5-propynyl cytosine; a 6-azo uracil; a 6-azo cytosine; a 6-azo thymine; a pseudouracil; a 4-thiouracil; an 8-haloadenin; an 8-aminoadenin; an 8-thioladenin; an 8-thioalkyladenin; an 8-hydroxyladenin; an 8-haloguanin; an 8-aminoguanin; an 8-thiolguanin; an 8-thioalkylguanin; an 8-hydroxylguanin; a 5-halouracil; a 5-bromouracil; a 5-trifluoromethyluracil; a 5-halocytosine; a 5-bromocytosine; a 5-trifluoromethylcytosine; a 5-substituted uracil; a 5-substituted cytosine; a 7-methylguanine; a 7-methyladenine; a 2-F-adenine; a 2-amino-adenine; an 8-azaguanine; an 8-azaadenine; a 7-deazaguanine; a 7-deazaadenine; a 3-deazaguanine; a 3-deazaadenine; a tricyclic pyrimidine; a phenoxazine cytidine; a phenothiazine cytidine; a substituted phenoxazine cytidine; a carbazole cytidine; a pyridoindole cytidine; a 7-deazaguanosine; a 2-aminopyridine; a 2-pyridone; a 5-substituted pyrimidine; a 6-azapyrimidine; an N-2, N-6 or 0-6 substituted purine; a 2-aminopropyladenine; a 5-propynyluracil; and a 5-propynylcytosine. Other versions of guide RNA may include other nucleic acids such as bridged nucleic acids or locked nucleic acids.

The guide RNAs described herein can further comprise one or more of a non-natural internucleoside linkage, a nucleic acid mimetic, a modified sugar moiety, and a modified nucleobase. In certain embodiments, the non-natural internucleoside linkage comprises one or more of: a phosphorothioate, a phosphoramidate, a non-phosphodiester, a heteroatom, a chiral phosphorothioate, a phosphorodithioate, a phosphotriester, an aminoalkylphosphotriester, a 3′-alkylene phosphonates, a 5′-alkylene phosphonate, a chiral phosphonate, a phosphinate, a 3′-amino phosphoramidate, an aminoalkylphosphoramidate, a phosphorodiamidate, a thionophosphoramidate, a thionoalkylphosphonate, a thionoalkylphosphotriester, a selenophosphate, and a boranophosphate. In certain embodiments, the nucleic acid mimetic comprises one or more of a peptide nucleic acid (PNA), morpholino nucleic acid, cyclohexenyl nucleic acid (CeNAs), or a locked nucleic acid (LNA). IN certain embodiments, the modified sugar moiety comprises one or more of 2′-O-(2-methoxyethyl), 2′-dimethylaminooxyethoxy, 2′-dimethylaminoethoxyethoxy, 2′-O-methyl, and 2′-fluoro. In certain embodiments the modified nucleobase comprises one or more of a 5-methylcytosine; a 5-hydroxymethyl cytosine; a xanthine; a hypoxanthine; a 2-aminoadenine; a 6-methyl derivative of adenine; a 6-methyl derivative of guanine; a 2-propyl derivative of adenine; a 2-propyl derivative of guanine; a 2-thiouracil; a 2-thiothymine; a 2-thiocytosine; a 5-halouracil; a 5-halocytosine; a 5-propynyl uracil; a 5-propynyl cytosine; a 6-azo uracil; a 6-azo cytosine; a 6-azo thymine; a pseudouracil; a 4-thiouracil; an 8-halo; an 8-amino; an 8-thiol; an 8-thioalkyl; an 8-hydroxyl; a 5-halo; a 5-bromo; a 5-trifluoromethyl; a 5-substituted uracil; a 5-substituted cytosine; a 7-methylguanine; a 7-methyladenine; a 2-F-adenine; a 2-amino-adenine; an 8-azaguanine; an 8-azaadenine; a 7-deazaguanine; a 7-deazaadenine; a 3-deazaguanine; a 3-deazaadenine; a tricyclic pyrimidine; a phenoxazine cytidine; a phenothiazine cytidine; a substituted phenoxazine cytidine; a carbazole cytidine; a pyridoindole cytidine; a 7-deaza-adenine; a 7-deazaguanosine; a 2-aminopyridine; a 2-pyridone; a 5-substituted pyrimidine; a 6-azapyrimidine; an N-2, N-6 or 0-6 substituted purine; a 2-aminopropyladenine; a 5-propynyluracil; and a 5-propynylcytosine. Exemplary guide RNAS of the present disclosure can be found in Table 4.

### Production of Chimeric Nucleases

Chimeric nucleases described herein may be produced in many ways including using an E. coli expression system as described in WO2020225719A1. Alternatively, the chimeric nucleases may be produced by the target cell to be modified by supplying one or more genetic vectors that directs expression and production of the nucleases in the target cell. Additionally, the vector may provide sequences to direct expression of guide RNAs to target the chimeric nuclease to particular genomic region.

An exemplary method for producing a genetically engineered cell as described herein is described below.

A population of cells is grown in a T flask to 70-90% confluency. The cells are harvested by centrifugation and resuspended to 1.0×10⁷ cells per milliliter (practical range 0.2-2×10⁷ cells per milliliter) in Buffer T (Invitrogen, Carlsbad, California, US). Cells are electroporated with a chimeric nuclease described herein including those selected from SEQ ID NOs: 750-756 and formulated in a Tris(hydroxymethyl)aminomethane or phosphate buffered saline with a Neon Transfection System (Thermo Fisher Scientific, Waltham, Massachusetts, US) at 2000 volts (practical range 1100-2500), 20 milliseconds (practical range 10-30 milliseconds) and 1 pulse (practical range 1-4). Cells are recovered in RPMI 1640 with 0.3 g/L glutamine and 2 g/L glucose (Sigma-Aldrich, Irvine, UK), 10% fetal bovine serum (Sigma-Aldrich, Oakville, Ontario, CA), 2 mM L-glutamine, and 100 units penicillin and 0.1 mg streptomycin/mL (Sigma-Aldrich, St. Louis, Missouri, US) for 24 hours. Dead cells are removed using a Dead Cell Removal Kit (Miltenyi Biotec, Somerville, Massachusetts, US). Knockout efficiency is measured by amplifying the target genes by polymerase chain reaction and measuring the proportion of cells edited by targeted amplicon sequencing (GENEWIZ, South Plainfield, NJ, US). Amplicon sequencing is a method of targeted next generation sequencing that enables you to analyze genetic variation in specific genomic regions. This method uses PCR to create sequences of DNA called amplicons. Amplicons from different samples can be multiplexed, also called indexed or pooled, which involves adding a barcode (index) to samples so they can be identified. Before multiplexing, individual samples used for amplicon sequencing must be transformed into libraries by adding adapters and enriching target regions via PCR amplification. The adapters allows formation of indexed amplicons and allow the amplicons to adhere to the flow cell for sequencing. Amplicon sequencing is typically used for variant detection in a population of cells.

Other methods to deliver the nuclease to the cell may be used, such as a lipid nanoparticle, polymer, viral vector or cell penetrating peptides. The chimeric nuclease or guide RNA may be delivered separately or in combination as DNA or RNA in either single-stranded or double-stranded form. Further, the chimeric nuclease may be delivered as RNA containing one or more of the following elements: a 5′ cap, a 5′ untranslated region, a coding sequence, a 3′ untranslated region and a poly adenine (poly-A) tail. The RNA might include different nucleobases for stability including, but not limited to, a 5-methylcytosine; a 5-hydroxymethyl cytosine; a xanthine; a hypoxanthine; a 2-aminoadenine; a 6-methyl derivative of adenine; a 6-methyl derivative of guanine; a 2-propyl derivative of adenine; a 2-propyl derivative of guanine; a 2-thiouracil; a 2-thiothymine; a 2-thiocytosine; a 5-propynyl uracil; a 5-propynyl cytosine; a 6-azo uracil; a 6-azo cytosine; a 6-azo thymine; a pseudouracil; a 4-thiouracil; an 8-haloadenin; an 8-aminoadenin; an 8-thioladenin; an 8-thioalkyladenin; an 8-hydroxyladenin; an 8-haloguanin; an 8-aminoguanin; an 8-thiolguanin; an 8-thioalkylguanin; an 8-hydroxylguanin; a 5-halouracil; a 5-bromouracil; a 5-trifluoromethyluracil; a 5-halocytosine; a 5-bromocytosine; a 5-trifluoromethylcytosine; a 5-substituted uracil; a 5-substituted cytosine; a 7-methylguanine; a 7-methyladenine; a 2-F-adenine; a 2-amino-adenine; an 8-azaguanine; an 8-azaadenine; a 7-deazaguanine; a 7-deazaadenine; a 3-deazaguanine; a 3-deazaadenine; a tricyclic pyrimidine; a phenoxazine cytidine; a phenothiazine cytidine; a substituted phenoxazine cytidine; a carbazole cytidine; a pyridoindole cytidine; a 7-deazaguanosine; a 2-aminopyridine; a 2-pyridone; a 5-substituted pyrimidine; a 6-azapyrimidine; an N-2, N-6 or 0-6 substituted purine; a 2-aminopropyladenine; a 5-propynyluracil; and a 5-propynylcytosine.

In another embodiment, the chimeric nuclease may be delivered as an integrating vector including, but not limited to retrovirus vectors, lentivirus vectors, transposon vectors, and adeno-associated virus vectors. The chimeric nuclease may also be delivered by other electroporation systems, including but not limited to a Nucleofector™ (Lonza, Basel, Switzerland), MaxCyte (Gaithersburg, MD) or CliniMACS@ (Bergisch Gladbach, Germany).

The chimeric nucleases may further be included in a pharmaceutical composition comprising one or more of a pharmaceutically acceptable carrier, diluent, or excipient. The term “pharmaceutically acceptable excipient,” as used herein, refers to carriers and vehicles that are compatible with the active ingredient (for example, a compound of the invention) of a pharmaceutical composition of the invention (and preferably capable of stabilizing it) and not deleterious to the individual to be treated. For example, solubilizing agents that form specific, more soluble complexes with the compounds of the invention can be utilized as pharmaceutical excipients for delivery of the compounds. Suitable carriers and vehicles are known to those of extraordinary skill in the art. The term “excipient” as used herein will encompass all such carriers, adjuvants, diluents, solvents, or other inactive additives. Pharmaceutical formulations may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, glycerol, tetrahydrofuryl alcohol, and fatty acid esters of sorbitan, cyclodextrins, albumin, hyaluronic acid, chitosan and mixtures thereof. Polyethylene glycol (PEG) may be used to obtain desirable properties of solubility, stability, half-life and other pharmaceutically advantageous properties. Representative examples of stabilizing components include polysorbate 80, L-arginine, polyvinylpyrrolidone, trehalose, and combinations thereof. Other excipients that may be employed, such as solution binders or anti-oxidants include, but are not limited to, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C and xylitol.

The guide RNAs of the present disclosure hybridize to a target sequence. In some embodiments, the target sequence is an oncogenic mutation. In some embodiments, the oncogenic mutation is selected from any one of KRAS, PIK3CA, EGFR, Muc4, or a combination thereof.

### Method of Targeting an Oncogenic Mutation

In some embodiments, provided herein are methods for targeting an oncogenic mutation in a cell, wherein the method comprises contacting the chimeric nuclease of the present disclosure to the cell. In some embodiments, provided herein are methods for targeting an oncogenic mutation. In some embodiments, the method comprises administering the chimeric nuclease composition of the present disclosure to an individual with a disease or disorder, thereby treating the disease or disorder. In some embodiments, the disease or disorder is cancer.

In some embodiments, the method comprises administering the chimeric nuclease composition of the present disclosure to an individual with cancer, thereby treating the cancer. The method comprising administering to an individual a therapeutically effective amount of a chimeric nuclease composition, or a pharmaceutical composition comprising a chimeric nuclease composition as described herein. In some embodiments, administration of the chimeric nuclease composition results in incorporation of one or more intended nucleotide edits in the target gene in the individual or cell. In some embodiments, administration of the chimeric nuclease results in correction of one or more oncogenic mutations, e.g., point mutations, insertions, or deletions, associated with an oncogene in the individual or cell.

The present disclosure provides a method of targeting an oncogenic mutation in a cell. The present disclosure also provides a method of targeting an oncogenic mutation in a individual. In some embodiments. In some embodiments, the cell is a cell in an individual afflicted with cancer. In some embodiments, the individual has cancer.

The present disclosure also provide the use of the chimeric nuclease composition for targeting the oncogenic mutation in a cell. The present disclosure provides a method of editing a genome in a cell. The present disclosure also provides a method of editing a genome in an individual. The present disclosure also provide the use of the chimeric nuclease composition for targeting the oncogenic mutation in an individual. In some embodiments, the cell is a cell in an individual afflicted with cancer. In some embodiments, the individual is an individual who has cancer. In some embodiments, the individual is undergoing a treatment which may induce metastasis. In some embodiments, the treatment comprises surgery, radiation treatment and chemotherapy. In some embodiments, the individual is a human. In some embodiments, the cancer is a carcinoma or a sarcoma. In some embodiments, the carcinoma comprises breast cancer, lung cancer, colon cancer, or prostate cancer. In some embodiments, the sarcoma comprises an osteosarcoma or a soft tissue sarcoma. In some embodiments, the cancer is a glioblastoma.

The present disclosure also provide the use of the chimeric nuclease composition for editing a genome in a cell. The present disclosure also provide the use of the chimeric nuclease composition for editing a genome in a individual. In some embodiments, the cell is a cell in an individual afflicted with cancer. In some embodiments, the individual is an individual who has cancer.

The present disclosure provides a method of deleting at least a portion of an oncogene in a cell. The present disclosure also provides a method of deleting at least a portion of an oncogene in a individual. In some embodiments. In some embodiments, the cell is a cell in an individual afflicted with cancer. In some embodiments, the individual is an individual who has cancer.

The present disclosure also provide the use of the chimeric nuclease composition for deleting at least a portion of an oncogene in a cell. The present disclosure also provide the use of the chimeric nuclease composition for deleting at least a portion of an oncogene in an individual. In some embodiments, the cell is a cell in an individual afflicted with cancer. In some embodiments, the individual is an individual who has cancer.

The present disclosure provides a method of silencing or disrupting at least a portion of an oncogene in a cell. The present disclosure also provides a method of silencing or disrupting at least a portion of an oncogene in a individual. In some embodiments. In some embodiments, the cell is a cell in an individual afflicted with cancer. In some embodiments, the individual is an individual who has cancer.

The present disclosure also provide the use of the chimeric nuclease composition for silencing or disrupting at least a portion of an oncogene in a cell. The present disclosure also provide the use of the chimeric nuclease composition for silencing or disrupting at least a portion of an oncogene in a individual. In some embodiments, the cell is a cell in an individual afflicted with cancer. In some embodiments, the individual is an individual who has cancer.

The present disclosure provides a method of replacing at least a portion of an oncogene in a cell. The present disclosure also provides a method of replacing at least a portion of an oncogene in a individual. In some embodiments. In some embodiments, the cell is a cell in an individual afflicted with cancer. In some embodiments, the individual is an individual who has cancer.

The present disclosure also provide the use of the chimeric nuclease composition for replacing at least a portion of an oncogene in a cell. The present disclosure also provide the use of the chimeric nuclease composition for replacing at least a portion of an oncogene in a individual. In some embodiments, the cell is a cell in an individual afflicted with cancer. In some embodiments, the individual is an individual who has cancer.

The present disclosure provides a method of restoring a non-oncogenic function in a cell. The present disclosure also provides a method restoring a non-oncogenic function in an individual. In some embodiments. In some embodiments, the cell is a cell in an individual afflicted with cancer. In some embodiments, the individual is an individual who has cancer.

The present disclosure also provide the use of the chimeric nuclease composition for restoring a non-oncogenic function in a cell. The present disclosure also provide the use of the chimeric nuclease composition for restoring a non-oncogenic function in an individual. In some embodiments, the cell is a cell in an individual afflicted with cancer. In some embodiments, the individual is an individual who has cancer.

Chimeric nucleases of this disclosure may be formulated for treating an individual (e.g., a human) having a disorder associated with pathological angiogenesis (e.g., cancer, such as breast cancer, ovarian cancer, renal cancer, colorectal cancer, liver cancer, gastric cancer, and lung cancer; obesity; macular degeneration; diabetic retinopathy; psoriasis; rheumatoid arthritis; cellular immunity; and rosacea.

In some embodiments, the cancer treated with the chimeric nuclease is selected from any one of prostate cancer, liver cancer, colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung, non-small cell lung), sarcoma (soft tissue sarcoma and osteosarcoma), melanoma, non melanoma skin cancer (squamous and basal cell carcinoma), glioma, renal cancer, lymphoma (NHI or HL), Acute myeloid leukemia (AML), T cell Acute Lymphoblastic Leukemia (T-ALL), Diffuse Large B cell lymphoma, testicular germ cell tumors, mesothelioma, esophageal cancer, Merkel Cells cancer, MSI-bigh cancer, KRAS mutant tumors, adult T-cell leukemia/lymphoma, and Myelodysplastic syndromes (MDS). In some embodiments of the method, the cancer is selected from any one of cancer triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, Myelodysplastic syndromes (MDS), or a combination thereof. In some embodiments of the method, the cancer is selected horn the group consisting of cancer triple negative breast cancer, stomach (gastric) cancer, lung cancer (small cell lung, non-srnall cell lung), Merkel Cells cancer, MSI-high cancer, or a combination thereof. In certain embodiments, the cancer includes a BRAF mutation (e.g., a BRAF V600E mutation), a BRAF wildtype, a KRAS wildtype or an activating KRAS mutation. The cancer may be at an early, intermediate or late stage.

In some embodiments, the method provided herein comprises administering to an individual an effective amount of a chimeric nuclease composition. In some embodiments, the method comprises administering to the individual an effective amount of a chimeric nuclease composition described herein, for example, polynucleotides, vectors, or constructs that encode chimeric nuclease components, LNPs, and/or polypeptides comprising chimeric nuclease components. Chimeric nuclease compositions can be administered to target an oncogene in a individual. Identifying a individual in need of such treatment can be in the judgment of a individual or a health care professional and can be subjective (e.g. opinion) or objective (e.g. measurable by a test or diagnostic method).

In some embodiments, the method comprises directly administering chimeric nuclease compositions provided herein to a individual. The chimeric nuclease compositions described herein can be delivered with in any form as described herein, e.g., as LNPs, RNPs, polynucleotide vectors such as viral vectors, or mRNAs. The chimeric nuclease compositions can be formulated with any pharmaceutically acceptable carrier described herein or known in the art for administering directly to a individual. Components of a chimeric nuclease composition or a pharmaceutical composition thereof may be administered to the individual simultaneously or sequentially. For example, in some embodiments, the method comprises administering a chimeric nuclease composition, or pharmaceutical composition thereof, comprising a chimeric nuclease to a individual. In some embodiments, the method comprises administering a polynucleotide or vector encoding a chimeric nuclease to a individual with a donor nucleic acid.

Suitable routes of administrating the chimeric nuclease to an individual include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the compositions described are administered intraperitoneally, intravenously, or by direct injection or direct infusion. In some embodiments, the compositions described herein are administered to a individual by injection, by means of a catheter, by means of a suppository, or by means of an implant.

In some embodiments, the method comprises administering cells edited with a chimeric nuclease composition described herein to an individual.

The specific dose administered can be a uniform dose for each individual. Alternatively, a individual's dose can be tailored to the approximate body weight of the individual. Other factors in determining the appropriate dosage can include the disease or condition to be treated or prevented, the severity of the disease, the route of administration, and the age, sex and medical condition of the patient.

In embodiments wherein components of a chimeric nuclease composition are administered sequentially, the time between sequential administration can be at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days.

In some embodiments, a method of monitoring treatment progress is provided. In some embodiments, the method includes the step of determining a level of diagnostic marker, for example, correction of a mutation in proto-oncogene.

### EXAMPLES

### Example 1: Generation of Disease-Causing Mutation Datasets

Provided herein, is a study utilizing a method to systematically scan a large dataset of mutant genes to identify putative allele-specific targets of chimeric nucleases using a computer algorithm is described. To increase the accuracy of Cas9 endonucleases, computer algorithms were developed to optimize guide RNA design and to predict potential target sites for nucleases have incorporated machine learning models to determine thermodynamic properties of target sites to better predict editing results. None of these algorithms identified allele-specific targets of chimeric nucleases.

The present disclosure provides a method to systematically identify allele-specific targets by first generating a curated dataset of mutant and non-mutant alleles from databases of disease-causing mutations, for example the NCBI database, cancer genome atlas database. Here, the genomic location of the disease-causing mutations was pulled from the dataset using a custom Python script and was used as a query for the GRCh38.p13 human genome. The 50 nucleotides surrounding the mutation were called and printed into a table format and the location of the mutation annotated in the sequence. The result was a pair of sequences—one with the wild type sequence and a second with the mutation annotated. The process was repeated for the subsequent mutations in the dataset.

### Identification of Allele-Specific Targets Using a Predictive Algorithm

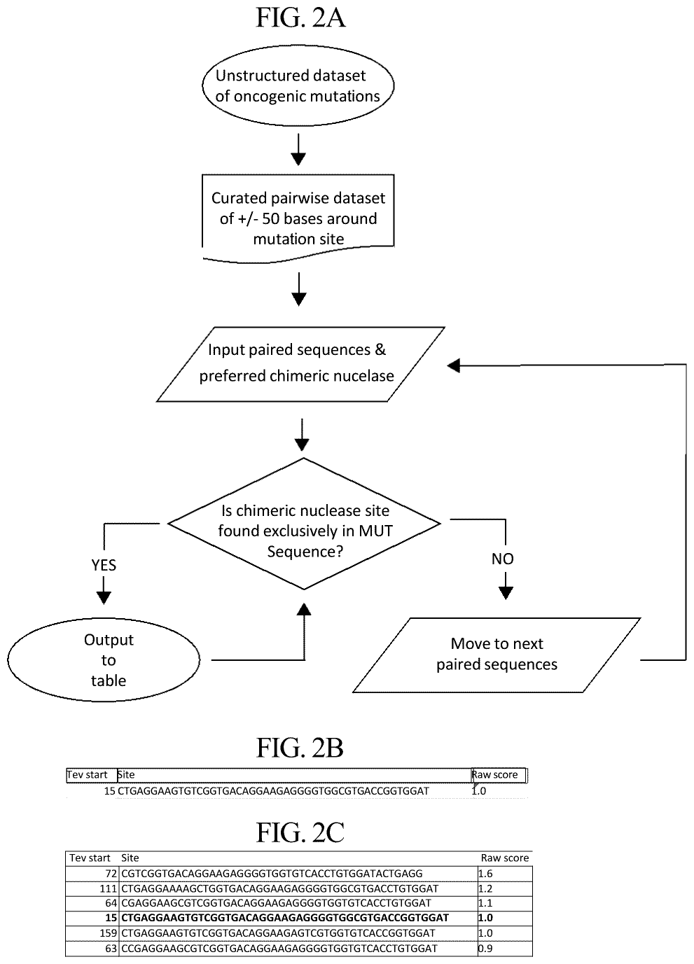

The method was used computationally to predict allele-specific chimeric nuclease sites. Specifically, and as illustrated in FIG. 2A, a custom coded Python script was created that uses the pairwise mutant and wild type sequences generated as described above as an input. A user selects from a set of chimeric nuclease parameters that have associated position-weighted matrices for the binding and cleavage preference of the I-TevI site, linker and Cas9 site. The algorithm then inputs a pair of wild type and mutant sequences from the dataset and identifies and scores chimeric nuclease sites. The sites found in the mutant sequence and compared to the wild type sequence and those sites that are exclusive to the mutant sequence, or are allele-specific, are printed to a line in a table. The script then proceeds to the next set of wild type and mutant sequences in the dataset until all paired sequences have been compared. The output consists of a table of predicted chimeric nuclease sites in which the nuclease targets a mutant sequence but not the wild type.

The development of the predictive algorithm allows for the systematic identification of allele-specific chimeric nuclease targets for potential therapeutic gene editing and replaces a previously manual process. In this example, 682 potential TevSaCas9 of SEQ ID Nos: 1-682 have been identified from the top 1000 most frequent cancer-causing mutations in the Genomic Data Commons (GDC) Data Portal where a single nucleotide polymorphism, a deletion or an insertion are the oncogenic mutation. Each of these mutants may be targets for therapeutic gene editing as a treatment for cancer. On such insertion mutant was computationally predicted to contain allele-specific chimeric nuclease sites in a large in-frame insertion mutation of the mucin-4 (Muc4) gene (FIG. 2B and FIG. 2C).

For the generation of FIG. 2B, a pair of wild type (WT) and mutant (MUT) sequences that includes 50 bases upstream and downstream of the mutation site are called from the GRCh38.p13 human genome sequence are input into the algorithm along with selected chimeric nuclease parameters, e.g., preferred I-TevI domain, linker and Cas9 domain. The sequence preferences consistent of a position-weighted matrix of the known binding and cleavage preferences of each domain relative to the native preference from in vitro data. The algorithm will identify chimeric nuclease sites that are present in the mutant sequence but not in the wild type sequence and each site found is output to a line in a table with the position-weighted matrix score. Once all sites are identified for the given pair of sequences, the algorithm will cycle to the next pair of mutant and wild type sequences until all pairs in the input dataset are analyzed.

### Example 2: Activity of Chimeric Nucleases to Selectively Target an Insertion Oncogene

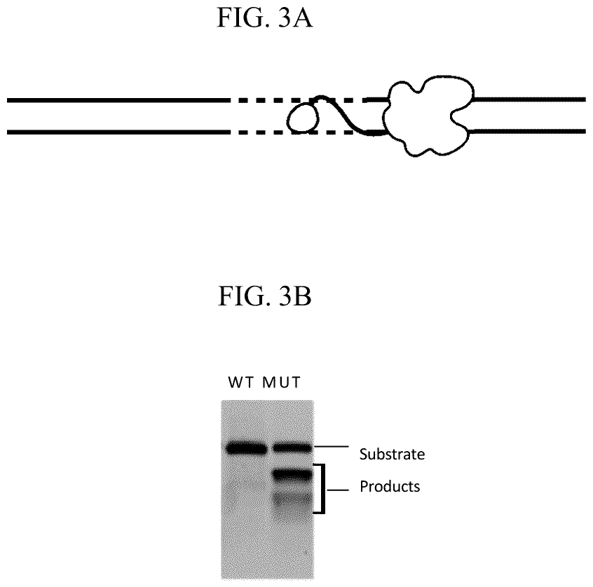

In this study, a chimeric nuclease was designed to target a large in-frame oncogenic insertion in the mucin-4 (Muc4) gene termed or the chr3:g.195781031_195781032insACCGGTGGATGCCGAGGAAGCGTCGGTGACAGGAAG AGGGGTGGTGTCACCTGTGGATACTGAGGAAAAGCTGGTGACAGGAAGAGGGGTGG CGTGACCTGTGGATACTGAGGAAGTGTCGGTGACAGGAAGAGTCGTGGTGTC (SEQ ID NO: 780) mutation. Mucin-4 is implicated in a variety of cancers and in particular colon cancer. Selective disruption of an insertion mutation in Muc4 using a chimeric nuclease to generate an out-of-frame deletion could provide a therapeutic benefit to patients. As illustrated in FIG. 3A the chimeric nuclease was targeted to Muc4 using the guide RNA of SEQ ID NO: 1685 which targets the TevSaCas9 chimeric nuclease to the insertion mutation but does not target wild type Muc4. The TevSaCas9 chimeric nuclease was purified from Escherichia coli and complexed with the guide RNA of SEQ ID NO: 1685. The complex was mixed with DNA substrate in an in vitro cleavage assay that contains theMuc4 insertion mutation in one reaction and wild type Muc4 sequence in a second reaction. FIG. 3B shows the results of an in vitro cleavage assay as visualized using agarose gel electrophoresis. It demonstrates TevSaCas9 that is programmed to Muc4 insertion preferentially cuts the oncogenic mutation sequence (presence of cleaved products), but not the wild type Muc4 sequence (no cleaved products).

### Example 3: Activity of Chimeric Nucleases to Selectively Eliminate Cancer Cells

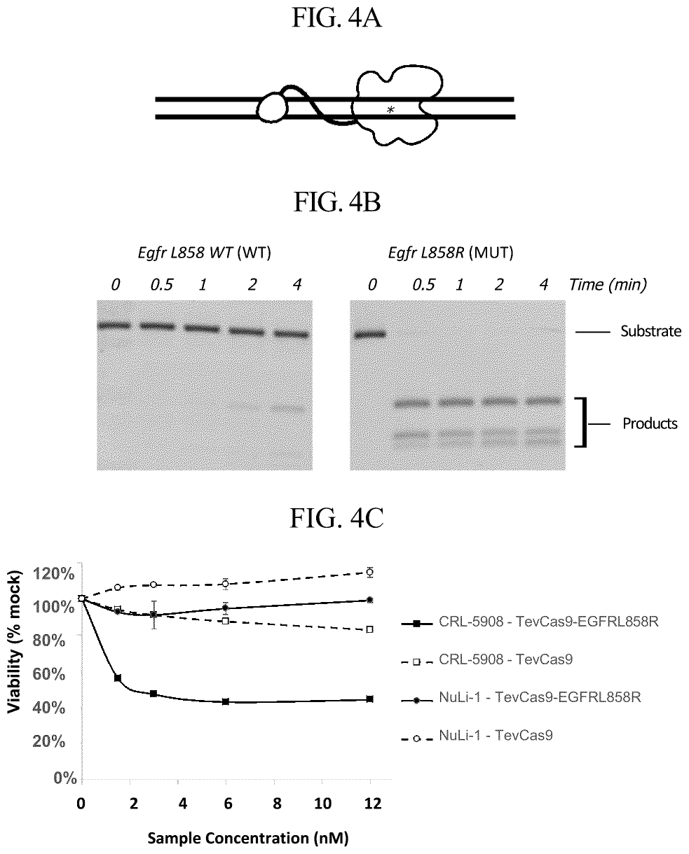

In another aspect of the invention, a chimeric nuclease is designed to target the Egfr L858R oncogenic activating mutation to selectively eliminate cancer cells. The Egfr L858R mutation is known to cause tumorigenesis and malignancy in non-small cell lung carcinoma (NSCLC). Targeting and eliminating the activating mutation could provide a therapeutic benefit for treating patients with cancer. As illustrated in FIG. 4A, the chimeric nuclease is targeted to the Egfr L858R mutation using the guide RNA of SEQ ID NO: 1686. The Egfr L858R mutation is located in the guide RNA binding site in the Egfr gene. Since precise binding of the guide RNA is required for TevSaCas9 activity, the mutant Egfr L858R gene can be discriminated from the wild type Egfr gene using the described nuclease. The TevSaCas9 chimeric nuclease is purified from Escherichia coli and complexed with the guide RNA of SEQ ID NO: 1686. The complex is mixed with DNA substrate in an in vitro cleavage assay amplified from cells containing the Egfr L858R mutation and in a separate reaction from cells that are wild type for Egfr. FIG. 4B shows the results of the in vitro cleavage assay over time as visualized using agarose gel electrophoresis. TevSaCas9 that is programmed to Egfr L858R preferentially cuts the Egfr L858R mutation over the wild type Egfr substrate. The described chimeric nuclease selectively eliminates cancer cells. CRL-5908 cells (American Type Culture Collection, Manassas, Virginia, United States of America) are an immortalized lung epithelial cell line that contains the Egfr L858R activating mutation. A control lung epithelial cell line NuLi-1 is wild type for the Egfr L858 amino acid. FIG. 4C demonstrates that CRL-5908 cells lipofected with TevSaCas9 targeted to the Egfr L858R mutation (“TevSaCas9-EGFRL858R”) with guide RNA of SEQ ID NO: 1686 have reduced viability of as measured by CellTiter-Blue viability dye (Promenga, Madison, Wisconsin, United State of America) compared to mock treated cells. NuLi-1 cell viability was not reduced by the same TevSaCas9-EGFRL858R complex nor was NuLi-1 cells or CRL-5908 cells treated with TevSaCas9 without guide RNA (“TevSaCas9”). Together, these data demonstrate allele-specific reduction in cell viability in human cells containing the Egfr L858R activating mutation by a chimeric nuclease targeted specifically to the mutation.

### Site Identification

Other embodiments of the method to identify sites include using a coding language other than Python to encode the algorithm to identify allele-specific targets. In a further embodiment, the chimeric nuclease parameters may include the binding and cleavage preference of orthologs of the I-TevI and/or the Cas9 domain.

Table 4 shows the output from the predictive algorithm of a set of putative TevSaCas9 sites in oncogenic mutations where nucleotide deletions are the driver mutation. Any of the sequences disclosed as a target site in Table 4 (SEQ ID NO: 1 to 683) can be targeted using the chimeric nucleases and methods described herein. Exemplary guide strands that can be used to target those target sites are also disclosed (SEQ ID NO: 1001 to 1686).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE 4
Sample output of Oncogenic mutations for use with the TevCas

| | | | | |
|---|---|---|---|---|
| Genome | | | Guide | |
| Location & | | SEQ ID | RNA | SEQ ID |
| Mutation | Target Site | NO: | sequence | NO: |

Single Nucleotide Polymorphisms

| | | | | |
|---|---|---|---|---|
| 18:51065549G > | CTGTGGACATTGGAGAGTTGAC | 1 | CCAAA | 1001 |
| A+ | CCAAACAAAAGtGATCTCCTCC | | CAAAA | |
| | AGAAG | | GtGATC | |
| | | | TCCTC | |
| 3:179199088G > | CAAAGTIGTCTTGTTTCATCAAA | 2 | AAATTC | 1002 |
| A+ | AAATTCTTCCCTTTCTGCTTCTT | | TTCCCT | |
| | GAGT | | TTCTGC | |
| | | | TTC | |
| 17:7674950A > C− | CGATGCTGAGGAGGGGCCAGA | 3 | TAAGA | 1003 |
| | CCTAAGAGCAATCAGTGAGGA | | GCAAT | |
| | ATCAGAGG | | CAGTG | |
| | | | AGGAA | |
| | | | T | |
| 10:46131074C > | CAGCGGTGGCCCaCAGCAGCTG | 4 | TGGCCC | 1004 |
| T+ | CTGGCCCAGGGACAGCTCAGT | | AGGGA | |
| | GCAGGGT | | CAGCTC | |
| | | | AGTG | |
| 3:179218294G > | CAAAGCAATTTCTACACGAGAT | 5 | CCTCTC | 1005 |
| A+ | CCTCTCTCTAAAATCACTGAGC | | TCTAAA | |
| | AGGAG | | ATCACT | |
| | | | GAG | |
| 3:179218307A > | CAAAGCAATTTCTACACGAGAT | 6 | CCTCTC | 1006 |
| G+ | CCTCTCTCTGAAATCACTGAGC | | TCTGAA | |
| | GGGAG | | ATCACT | |
| | | | GAG | |
| 3:179218303G > | CAAAGCAATTTCTACACGAGAT | 7 | CCTCTC | 1007 |
| A+ | CCTCTCTCTGAAATCACTAAGC | | TCTGAA | |
| | AGGAG | | ATCACT | |
| | | | AAG | |
| 3:179218304A > | CAAAGCAATTTCTACACGAGAT | 8 | CCTCTC | 1008 |
| C+ | CCTCTCTCTGAAATCACTGCGC | | TCTGAA | |
| | AGGAG | | ATCACT | |
| | | | GCG | |
| 17:4539347A > T− | CATGGTTTAATAAAAAAAAAA | 9 | AAAAT | 1009 |
| | AAAAATAGGCGTCTCAGGCAG | | AGGCG | |
| | ATGGAGG | | TCTCAG | |
| | | | GCAGA | |
| 3:195783009C > | CGTCGGTGACAGGAAGAGAGG | 10 | TGGCGT | 1010 |
| T− | TGGCGTGACCTGTGGATACTGA | | GACCT | |
| | GGAAG | | GTGGA | |
| | | | TACTG | |
| 3:195783008A > | CGTCGGTGACAGGAAGAGAGG | 11 | TGGCGT | 1011 |
| G− | TGGCGTGACCTGTGGGCACTGA | | GACCT | |
| | GGAAG | | GTGGG | |
| | | | CACTG | |
| 19:52212729C > | CTTGGCAAACTCCCCCAGCTTG | 12 | GAGGC | 1012 |
| T+ | GAGGCTGCGGCCCaCCGCACCA | | TGCGG | |
| | TGGGG | | CCCaCC | |
| | | | GCACC | |
| 17:4539347A > T− | CATGGTTTAATAAAAAAAAAA | 13 | AAAAA | 1013 |
| | AAAAATAGGCGTCTCAGGCAG | | TAGGC | |
| | ATGGAG | | GTCTCA | |
| | | | GGCAG | |
| 17:7674947A > G− | CTCGGGTAAGATGCTGAGGAG | 14 | GGCCA | 1014 |
| | GGGCCAGACCTAAGAGCAATC | | GACCT | |
| | AGTGAGG | | AAGAG | |
| | | | CAATC | |
| | | | A | |
| 17:7673704G > A− | CCAGGGAGCACTAAGtGAGGTA | 15 | AGCAA | 1015 |
| | AGCAAGCAGGACAAGAAGCGG | | GCAGG | |
| | TGGAGG | | ACAAG | |
| | | | AAGCG | |
| | | | G | |
| 19:52212729C > | CTTGGCAAACTCCCCCAGCTTG | 16 | AGGCT | 1016 |
| T+ | GAGGCTGCGGCCCaCCGCACCA | | GCGGC | |
| | TGGGGG | | CCaCCG | |
| | | | CACCA | |
| 7:56106490G> A− | CACCGAAGAGCTAAGCGACTT | 17 | GAGGA | 1017 |
| | CTGAGGAGACCGGAAAATGGG | | GACCG | |
| | AGGCGGGG | | GAAAA | |
| | | | TGGGA | |
| | | | G | |
| 12:56085070G > | CCTGGGTCCCTCGCACCAtGCG | 18 | AGGTT | 1018 |
| A+ | GAGGTTGGGCAATGGTAGAGT | | GGGCA | |
| | AGAGAAT | | ATGGT | |
| | | | AGAGT | |
| | | | A | |
| 10:121520163G > | CATCGCTCTGGTGGAGAGAGG | 19 | AGAAA | 1019 |
| C− | GAAGAAAGGAGGAGTGGGGAT | | GGAGG | |
| | GGGAGAAT | | AGTGG | |
| | | | GGATG | |
| | | | G | |
| 10:46131074C > | CTGAGCTGTCCCTGGGCCAGCA | 20 | AGCTG | 1020 |
| T+ | GCTGCTGTGGGCCACCGCTGAT | | CTGTGG | |
| | GAGG | | GCCAC | |
| | | | CGCTG | |
| 17:31350209C > | CACCGTTTTCCTTTTAGCTTTAC | 21 | ACTTAC | 1021 |
| T+ | TTACAGTGTCTGAAGAAGTTTG | | AGTGTC | |
| | AAG | | TGAAG | |
| | | | AAGT | |
| 10:121520163G > | CATCGCTCTGGTGGAGAGAGG | 22 | GGAAG | 1022 |
| C− | GAAGAAAGGAGGAGTGGGGAT | | AAAGG | |
| | GGGAG | | AGGAG | |
| | | | TGGGG | |
| | | | A | |
| 17:7674229C > A− | CATGGGCGtCATGAACCGGAGG | 23 | CCATCC | 1023 |
| | CCCATCCTCACCATCATCACAC | | TCACCA | |
| | TGGAAG | | TCATCA | |
| | | | CAC | |
| 6:26217357A > T+ | CATGGGCAAGTGAAATGATTA | 24 | TAGTCA | 1024 |
| | CTAGTCAAATCCGTCAGTGATC | | AATCC | |
| | CCGAGT | | GTCAGT | |
| | | | GATC | |
| 19:49665874C > | CTGGGCTGTTCCCGCCCCTATG | 25 | GCCCTT | 1025 |
| T+ | CCCTTTTTTGGGTTTTCGGCCA | | TTTTGG | |
| | GAGG | | GTTTTC | |
| | | | GGC | |
| 17:4539348A > T− | CATGGTTTAAATAAAAAAAAA | 26 | AAAAT | 1026 |
| | AAAAATAGGCGTCTCAGGCAG | | AGGCG | |
| | ATGGAGG | | TCTCAG | |
| | | | GCAGA | |
| 17:7674229C > T− | CATGGGCGaCATGAACCGGAGG | 27 | CCATCC | 1027 |
| | CCCATCCTCACCATCATCACAC | | TCACCA | |
| | TGGAAG | | TCATCA | |
| | | | CAC | |
| 17:7673704G > A− | CCAGGGAGCACTAAGtGAGGTA | 28 | AAGCA | 1028 |
| | AGCAAGCAGGACAAGAAGCGG | | AGCAG | |
| | TGGAG | | GACAA | |
| | | | GAAGC | |
| | | | G | |
| 3:179199690G > | CACTGGCATGCCGATAGCAAA | 29 | AAtCTT | 1029 |
| A+ | AtCTTTAATAAAATTACATAAA | | TAATA | |
| | GAAT | | AAATT | |
| | | | ACATA | |
| 3:41224633A > G+ | CTCTGGAATCCATTCTGGTGCC | 30 | GCCACT | 1030 |
| | ACTGCCACAGCTCCTTCTCTGA | | GCCAC | |
| | GT | | AGCTCC | |
| | | | TTCT | |
| 3:41224622C > T+ | CTCTGGAATCCATTTTGGTGCC | 31 | GCCACT | 1031 |
| | ACTACCACAGCTCCTTCTCTGA | | ACCAC | |
| | GT | | AGCTCC | |
| | | | TTCT | |
| 17:4539348A > T− | CATGGTTTAAATAAAAAAAAA | 32 | AAAAA | 1032 |
| | AAAAATAGGCGTCTCAGGCAG | | TAGGC | |
| | ATGGAG | | GTCTCA | |
| | | | GGCAG | |
| 3:179218304A > | CGCAGGAGAAAGATTTTCTATG | 33 | GAGTC | 1033 |
| C+ | GAGTCACAGGTAAGTGCTAAA | | ACAGG | |
| | ATGGAG | | TAAGT | |
| | | | GCTAA | |
| | | | A | |
| 3:41224645T > C+ | CTCTGGAATCCATTCTGGTGCC | 34 | GCCACT | 1034 |
| | ACTACCACAGCTCCTCCTCTGA | | ACCAC | |
| | GT | | AGCTCC | |
| | | | TCCT | |
| 17:7675190C > T− | CAGGGTAGGTCTTGGCCAGTTG | 35 | CAAAA | 1035 |
| | GCAAAACATCTTGTTGAGGGCA | | CATCTT | |
| | GGGGAG | | GTTGA | |
| | | | GGGCA | |
| 17:7675190C > T− | CGCGGGTGCCGGGCGGGGGTG | 36 | TGGAA | 1036 |
| | TGGAATCAACCCACAGCTGCAC | | TCAACC | |
| | AGGGT | | CACAG | |
| | | | CTGCA | |
| 12:25225628C > | CAGTGTTACTTACCTGTCTTGT | 37 | CTTTGT | 1037 |
| T− | CTTTGTTGATGTTTCAATAAAA | | TGATGT | |
| | GGAAT | | TTCAAT | |
| | | | AAA | |
| 6:26217357A > T+ | CCATGGGCAAGTGAAATGATT | 38 | TAGTCA | 1038 |
| | ACTAGTCAAATCCGTCAGTGAT | | AATCC | |
| | CCCGAGT | | GTCAGT | |
| | | | GATC | |
| 17:7674945G > A− | CTCAGATAAGATGCTGAGGAG | 39 | GGCCA | 1039 |
| | GGGCCAGACCTAAGAGCAATC | | GACCT | |
| | AGTGAGG | | AAGAG | |
| | | | CAATC | |
| | | | A | |
| 17:7675190C > T− | CAGGGTAGGTCTTGGCCAGTTG | 40 | GGCAA | 1040 |
| | GCAAAACATCTTGTTGAGGGCA | | AACAT | |
| | GGGG | | CTTGTT | |
| | | | GAGGG | |
| 10:45827364T > | CACAGGTTTCCACCGTGCACAT | 41 | TATGA | 1041 |
| G− | TATGAAGAACAGAAATGGAGG | | AGAACA | |
| | TGGGAG | | GAAAT | |
| | | | GGAGG | |
| 12:25227342T > | CTTGGATATTCTCGACACAGCA | 42 | GGTCgA | 1042 |
| C− | GGTCgAGAGGAGTACAGTGCA | | GAGGA | |
| | ATGAGG | | GTACA | |
| | | | GTGCA | |
| 17:7673704G > A− | CACCGCTTCTTGTCCTGCTTGCT | 43 | CTTACC | 1043 |
| | TACCTCACTTAGTGCTCCCTGG | | TCACTT | |
| | GGG | | AGTGCT | |
| | | | CCC | |
| 17:7674216C > A− | CATGGGCGGCATGAACCGGAGt | 44 | CCATCC | 1044 |
| | CCCATCCTCACCATCATCACAC | | TCACCA | |
| | TGGAAG | | TCATCA | |
| | | | CAC | |
| 7:55191822T > G | CACCGCAGCATGTCAAGATCAC | 45 | GATTTT | 1045 |
| + | AGATTTTGGGCGGGCCAAACTG | | GGGCG | |
| | CTGGGT | | GGCCA | |
| | | | AACTG | |
| 17:39711955C > | CGAGGGTGCAGaATCCCACGTC | 46 | TCCGTA | 1046 |
| T+ | CGTAGAAAGGTAGTTGTCTAAG | | GAAAG | |
| | GAG | | GTAGTT | |
| | | | GTCT | |
| 10:79514306C > | CTCTGCTTCTTTaCATCTCAGTT | 47 | GTTAGT | 1047 |
| T+ | AGTCCCTTCTGAAATTCAGTGA | | CCCTTC | |
| | GG | | TGAAA | |
| | | | TTCA | |
| 3:195778903G > | CTGAGGAAAGGCTGGTGACAT | 48 | TGAAG | 1048 |
| T− | GAAGAGGGGTGGCGTGACCTG | | AGGGG | |
| | TGGAT | | TGGCGT | |
| | | | GACCT | |
| 17:7674220C > T− | CATGGGCGGCATGAACCaGAGG | 49 | CCATCC | 1049 |
| | CCCATCCTCACCATCATCACAC | | TCACCA | |
| | TGGAAG | | TCATCA | |
| | | | CAC | |
| 17:7674221G > A− | CATGGGCGGCATGAACtGGAGG | 50 | CCATCC | 1050 |
| | CCCATCCTCACCATCATCACAC | | TCACCA | |
| | TGGAAG | | TCATCA | |
| | | | CAC | |
| 12:25227342T > | CTTGGATATTCTCGACACAGCA | 51 | GGTCtA | 1051 |
| A− | GGTCtAGAGGAGTACAGTGCAA | | GAGGA | |
| | TGAGG | | GTACA | |
| | | | GTGCA | |
| 12:25227341T > | CTTGGATATTCTCGACACAGCA | 52 | GGTCAc | 1052 |
| G− | GGTCACGAGGAGTACAGTGCAA | | GAGGA | |
| | TGAGG | | GTACA | |
| | | | GTGCA | |
| 10:45827364T > | CACAGGTTTCCACCGTGCACAT | 53 | ATGAA | 1053 |
| G− | TATGAAGAACAGAAATGGAGG | | GAAcAG | |
| | TGGGAGT | | AAATG | |
| | | | GAGGT | |
| 10:87957915C > | CAAAGTACATGAACTTGTCTTC | 54 | CCCGTC | 1054 |
| T+ | CCGTCaTGTGGGTCCTGAATTG | | aTGTGG | |
| | GAGG | | GTCCTG | |
| | | | AAT | |
| 17:7673579G > A− | CCTAGCACTGCCCAACAACACC | 55 | ACCAG | 1055 |
| | AGCTCCTCTCCCtAGCCAAAGA | | CTCCTC | |
| | AG | | TCCCtA | |
| | | | GCCA | |
| 17:7675994C > A− | CTCAGGGCAACTGACAGTGCA | 56 | AAGTC | 1056 |
| | AGTCACAGACTTGGCTGTCCCA | | ACAGA | |
| | GAAT | | CTTGGC | |
| | | | TGTCC | |
| 17:7673704G > A− | CACCGCTTCTTGTCCTGCTTGCT | 57 | GCTTAC | 1057 |
| | TACCTCACTTAGTGCTCCCTGG | | CTCACT | |
| | GG | | TAGTGC | |
| | | | TCC | |
| 17:7673767C > T− | CCTGGGAGAGACCGGCGCACAa | 58 | AGGAA | 1058 |
| | AGGAAGAGAATCTCCGCAAGA | | GAGAA | |
| | AAGGGG | | TCTCCG | |
| | | | CAAGA | |
| 17:7674953T > C− | CACTGATTGCTCTTAGGTCTGG | 59 | GGCCC | 1059 |
| | CCCCTCCTCAGCgTCTTATCCGA | | CTCCTC | |
| | GT | | AGCgTC | |
| | | | TTAT | |
| 17:7674950A > C− | CACTGATTGCTCTTAGGTCTGG | 60 | GGCCC | 1060 |
| | CCCCTCCTCAGCATCgTATCCG | | CTCCTC | |
| | AGT | | AGCAT | |
| | | | CgTAT | |
| 17:7673776G > A− | CCTGGGAGAGACIGGCGCACAG | 61 | AGGAA | 1061 |
| | AGGAAGAGAATCTCCGCAAGA | | GAGAA | |
| | AAGGGG | | TCTCCG | |
| | | | CAAGA | |
| 12:25227342T > | CACAGCAGGTCgAGAGGAGTA | 62 | AGTGC | 1062 |
| C− | CAGTGCAATGAGGGACCAGTA | | AATGA | |
| | CATGAGG | | GGGAC | |
| | | | CAGTA | |
| | | | C | |
| 12:25227341T > | CACAGCAGGTCAcGAGGAGTAC | 63 | AGTGC | 1063 |
| G− | AGTGCAATGAGGGACCAGTAC | | AATGA | |
| | ATGAGG | | GGGAC | |
| | | | CAGTA | |
| | | | C | |
| 17:7675085C > A− | CCCAGCTGCTCACCATCGCTAT | 64 | TCTGAG | 1064 |
| | CTGAGCAGCGCTCATGGTGGG | | CAGCG | |
| | GGAAG | | CTCATG | |
| | | | GTGG | |
| 17:7673781C > T− | CTGGGAaAGACCGGCGCACAG | 65 | GAAGA | 1065 |
| | AGGAAGAGAATCTCCGCAAGA | | GAATCT | |
| | AAGGGGAG | | CCGCA | |
| | | | AGAAA | |
| 17:7673781C > T− | CTGGGAaAGACCGGCGCACAG | 66 | AGGAA | 1066 |
| | AGGAAGAGAATCTCCGCAAGA | | GAGAA | |
| | AAGGGG | | TCTCCG | |
| | | | CAAGA | |
| 3:195780140G > | CTGAGGAAAAGCTGGTGACAG | 67 | GGAAG | 1067 |
| A− | GAAGAGGGGTGGCCTGACCTG | | AGGGG | |
| | TGGAT | | TGGCCT | |
| | | | GACCT | |
| 10:79514306C > | CTCAGTTTCCTCACTGAATTTC | 68 | GAAGG | 1068 |
| T+ | AGAAGGGACTAACTGAGATGT | | GACTA | |
| | AAAGAAG | | ACTGA | |
| | | | GATGT | |
| | | | A | |
| 17:7674947A > G− | CACTGATTGCTCTTAGGTCTGG | 69 | GGCCC | 1069 |
| | CCCCTCCTCAGCATCTTAcCCG | | CTCCTC | |
| | AGT | | AGCAT | |
| | | | CTTAc | |
| 12:16040G > A− | CCCAGGGAAGTGGTtGACCCCT | 70 | CCGGT | 1070 |
| | CCGGTGGCTGGGCCACTCTGCT | | GGCTG | |
| | AGAGT | | GGCCA | |
| | | | CTCTGC | |
| 17:7674957G > A− | CACTGATTGCTCTTAGGTCTGG | 71 | GGCCC | 1071 |
| | CCCCTCCTAGCATCTTATCCGA | | CTCCTt | |
| | GT | | AGCAT | |
| | | | CTTAT | |
| 17:7673781C > G− | CTGGGAcAGACCGGCGCACAG | 72 | AGGAA | 1072 |
| | AGGAAGAGAATCTCCGCAAGA | | GAGAA | |
| | AAGGGG | | TCTCCG | |
| | | | CAAGA | |
| 1:247738656A > | CACAGATGATGGAATTCTTGCT | 73 | GCTTGT | 1073 |
| C+ | TGTGAGCTTTACTGAGAATTGG | | GAGCTT | |
| | GT | | TACTGA | |
| | | | GAA | |
| 17:7673764C > T− | CCTGGGAGAGACCGGCGCACA | 74 | AGaAAG | 1074 |
| | GAGaAAGAGAATCTCCGCAAG | | AGAAT | |
| | AAAGGGG | | CTCCGC | |
| | | | AAGA | |
| 17:7673781C > G− | CTGGGAcAGACCGGCGCACAG | 75 | GAAGA | 1075 |
| | AGGAAGAGAATCTCCGCAAGA | | GAATCT | |
| | AAGGGGAG | | CCGCA | |
| | | | AGAAA | |
| 12:16040G > A− | CCAGGGAAGTGGTtGACCCCTC | 76 | CCGGT | 1076 |
| | CGGTGGCTGGGCCACTCTGCTA | | GGCTG | |
| | GAGT | | GGCCA | |
| | | | CTCTGC | |
| 12:25227342T > | CACAGCAGGTCtAGAGGAGTAC | 77 | AGTGC | 1077 |
| A− | AGTGCAATGAGGGACCAGTAC | | AATGA | |
| | ATGAGG | | GGGAC | |
| | | | CAGTA | |
| | | | C | |
| 17:7674230C > T− | CATGGGCaGCATGAACCGGAGG | 78 | CCATCC | 1078 |
| | CCCATCCTCACCATCATCACAC | | TCACCA | |
| | TGGAAG | | TCATCA | |
| | | | CAC | |
| 17:7674957G > A− | CTCGGATAAGATGCTAAGGAG | 79 | GGCCA | 1079 |
| | GGGCCAGACCTAAGAGCAATC | | GACCT | |
| | AGTGAGG | | AAGAG | |
| | | | CAATC | |
| | | | A | |
| 10:87957915C > | CAAAGTACATGAACTTGTCTTC | 80 | TCCCGT | 1080 |
| T+ | CCGTCaTGTGGGTCCTGAATTG | | CaTGTG | |
| | GAG | | GGTCCT | |
| | | | GAA | |
| 17:7673781C > G− | CCTGGGAcAGACCGGCGCACAG | 81 | AGGAA | 1081 |
| | AGGAAGAGAATCTCCGCAAGA | | GAGAA | |
| | AAGGGG | | TCTCCG | |
| | | | CAAGA | |
| 17:7675139C > T− | CTGTGACTGCTTGTAGATGGCC | 82 | TGGCGT | 1082 |
| | ATGGCGTGGACGCGGGTGCCG | | GGACG | |
| | GGCGGGG | | CGGGT | |
| | | | GCCGG | |
| 17:7674953T > C− | CTCGGATAAGACGCTGAGGAG | 83 | GGCCA | 1083 |
| | GGGCCAGACCTAAGAGCAATC | | GACCT | |
| | AGTGAGG | | AAGAG | |
| | | | CAATC | |
| | | | A | |
| 3:195779688A > | CTGAGGAACGGCTGGTGACAG | 84 | GGAAG | 1084 |
| G− | GAAGAGAGGTGGCGTGGCCTG | | AGAGG | |
| | TGGAT | | TGGCGT | |
| | | | GGCCT | |
| 17:7675139C > A− | CTGTGACTGCTTGTAGATGGCC | 85 | TGGCG | 1085 |
| | ATGGCGAGGACGCGGGTGCCG | | AGGAC | |
| | GGCGGGG | | GCGGG | |
| | | | TGCCG | |
| | | | G | |
| 4:152326214C > | CAGAGTTGTTAGCGGTTCTCaA | 86 | CaAGAT | 1086 |
| T− | GATGCCACTCTTAGGGTTTGGG | | GCCACT | |
| | AT | | CTTAGG | |
| | | | GTT | |
| 17:7675143C > A− | CTGTGACTGCTTGTAGATGGCC | 87 | TGGCG | 1087 |
| | ATGGCGCGGAAGCGGGTGCCG | | CGGAA | |
| | GGCGGGG | | GCGGG | |
| | | | TGCCG | |
| | | | G | |
| 19:52212718C > | CTGCGGCCCGCCGCACCATGcG | 88 | GTGTCA | 1088 |
| G+ | GGTGTCATCTGAGCACAGGTTC | | TCTGAG | |
| | CGGAAG | | CACAG | |
| | | | GTTC | |
| 17:7673781C > T− | CCTGGGAaAGACCGGCGCACAG | 89 | AGGAA | 1089 |
| | AGGAAGAGAATCTCCGCAAGA | | GAGAA | |
| | AAGGGG | | TCTCCG | |
| | | | CAAGA | |
| 10:94402611C > | CCGAGCAGGCTCCGGCATCCTC | 90 | CTCCGG | 1090 |
| T+ | CGGACCCACCATCTaGGGGTGG | | ACCCA | |
| | GT | | CCATCT | |
| | | | aGGG | |
| 10:94402613G > | CCGAGCAGGCTCCGGCATCCTC | 91 | CTCCGG | 1091 |
| A+ | CGGACCCACCATtTGGGGGTGG | | ACCCA | |
| | GT | | CCATtT | |
| | | | GGGG | |
| 3:195783009C > | CTGAGGAAGCGTCGGTGACAG | 92 | GGAAG | 1092 |
| T− | GAAGAGAGGTGGCGTGACCTG | | AGAGG | |
| | TGGAT | | TGGCGT | |
| | | | GACCT | |
| 10:79513976C > | CACTGGCTTCTCATTTGCaTTAA | 93 | TAAACT | 1093 |
| T+ | ACTGTGAACTCCTTTAGGGTGG | | GTGAA | |
| | GG | | CTCCTT | |
| | | | TAGG | |
| 19:52212729C > | CCTTGGCAAACTCCCCCAGCTT | 94 | GAGGC | 1094 |
| T+ | GGAGGCTGCGGCCCaCCGCACC | | TGCGG | |
| | ATGGGG | | CCCaCC | |
| | | | GCACC | |
| 17:7670685G > A− | CTCAGAACATCTCGAAGCGCTC | 95 | ACGCC | 1095 |
| | ACGCCCACGGATCTGCAGCAA | | CACGG | |
| | CAGAGG | | ATCTGC | |
| | | | AGCAA | |
| 17:7675124T > C− | CATGGCCATCTgCAAGCAGTCA | 96 | TCACA | 1096 |
| | CAGCACATGACGGAGGTTGTG | | GCACA | |
| | AGG | | TGACG | |
| | | | GAGGT | |
| | | | T | |
| 17:7674947A > G− | CTCAGCATCTTAcCCGAGTGGA | 97 | GGAAA | 1097 |
| | AGGAAATTTGCGTGTGGAGTAT | | TTTGCG | |
| | TTGGAT | | TGTGG | |
| | | | AGTAT | |
| 10:87957915C > | CTGAGGGAACTCAAAGTACAT | 98 | ATGAA | 1098 |
| T+ | GAACTTGTCTTCCCGTCaTGTGG | | CTTGTC | |
| | GT | | TTCCCG | |
| | | | TCaT | |
| 1:144436988C > | CTTGGAGGTCCTGCCCCTGGGA | 99 | CTTGTC | 1099 |
| A− | CTTGTCCGGCTCATACGGAGTG | | CGGCTC | |
| | AGGAG | | ATACG | |
| | | | GAGT | |
| 17:7675085C > T− | CCCAGCTGCTCACCATCGCTAT | 100 | TATCTG | 1100 |
| | CTGAGCAGCGCTCATGGTGGG | | AGCAG | |
| | GGT | | CGCTCA | |
| | | | TGGT | |
| 19:52212729C > | CTGCGGCCCaCCGCACCATGGG | 101 | GTGTCA | 1101 |
| T+ | GGTGTCATCTGAGCACAGGTTC | | TCTGAG | |
| | CGGAAG | | CACAG | |
| | | | GTTC | |
| 17:7675124T > C− | CCATGGCCATCTgCAAGCAGTC | 102 | TCACA | 1102 |
| | ACAGCACATGACGGAGGTTGT | | GCACA | |
| | GAGG | | TGACG | |
| | | | GAGGT | |
| | | | T | |
| 19:52212729C > | CTCAGATGACACCCCCATGGTG | 103 | CGGTG | 1103 |
| T+ | CGGTGGGCCGCAGCCTCCAAG | | GGCCG | |
| | CTGGGG | | CAGCCT | |
| | | | CCAAG | |
| 3:195780075T > | CTGAGGAAGGGATGGTGACAG | 104 | GGAAG | 1104 |
| G− | GAAGAGGGGTGGCGTGACCGG | | AGGGG | |
| | TGGAT | | TGGCGT | |
| | | | GACCG | |
| 3:41224645T > C+ | CACAGCTCCTCCTCTGAGTGGT | 105 | GGTAA | 1105 |
| | AAAGGCAATCCTGAGGAAGAG | | AGGCA | |
| | GAT | | ATCCTG | |
| | | | AGGAA | |
| 17:7674950A > C− | CTCAGCATCgTATCCGAGTGGA | 106 | GGAAA | 1106 |
| | AGGAAATTTGCGTGTGGAGTAT | | TTTGCG | |
| | TTGGAT | | TGTGG | |
| | | | AGTAT | |
| 17:7674917T > C− | CTCAGCATCTTATCCGAGTGGA | 107 | GGAAA | 1107 |
| | AGGAAATTTGCGTGTGGAGTgT | | TTTGCG | |
| | TTGGAT | | TGTGG | |
| | | | AGTgT | |
| 17:7674950A > C− | CTCGGATACGATGCTGAGGAG | 108 | GGCCA | 1108 |
| | GGGCCAGACCTAAGAGCAATC | | GACCT | |
| | AGTGAGG | | AAGAG | |
| | | | CAATC | |
| | | | A | |
| 10:94402613G > | CTTTGCCTCCTGGGGCGGCCGC | 109 | ACCCA | 1109 |
| A+ | CACCCACCCCCAAATGGTGGGT | | CCCCCA | |
| | CCGGAG | | AATGG | |
| | | | TGGGT | |
| 17:7674953T > C− | CTCAGCgTCTTATCCGAGTGGA | 110 | GGAAA | 1110 |
| | AGGAAATTTGCGTGTGGAGTAT | | TTTGCG | |
| | TTGGAT | | TGTGG | |
| | | | AGTAT | |
| 10:94402611C > | CTTTGCCTCCTGGGGCGGCCGC | 111 | ACCCA | 1111 |
| T+ | CACCCACCCCTAGATGGTGGGT | | CCCCTA | |
| | CCGGAG | | GATGG | |
| | | | TGGGT | |
| 2:113595432G > | CTGTGGCGGGGGCGTCTCTGCA | 112 | AGGCC | 1112 |
| A+ | GGCCAGGGTCCTGGGCGCtCGT | | AGGGT | |
| | GAAG | | CCTGG | |
| | | | GCGCtC | |
| 17:7674945G > A− | CTCAGCATCTTATCtGAGTGGA | 113 | GGAAA | 1113 |
| | AGGAAATTTGCGTGTGGAGTAT | | TTTGCG | |
| | TTGGAT | | TGTGG | |
| | | | AGTAT | |
| 2:113595432G > | CTGGGCGCtCGTGAAGATGGAG | 114 | AGCCA | 1114 |
| A+ | CCATATTCCTGCAGGCGCTCTG | | TATTCC | |
| | GAG | | TGCAG | |
| | | | GCGCT | |
| 17:7675139C > A− | CCGCGTCCtCGCCATGGCCATCT | 115 | TACAA | 1115 |
| | ACAAGCAGTCACAGCACATGA | | GCAGT | |
| | CGGAG | | CACAG | |
| | | | CACAT | |
| | | | G | |
| 19:3118944A > T+ | CTGTGTCCTTTCAGGATGGTGG | 116 | TGTGG | 1116 |
| | ATGTGGGGGGCCTGCGGTCGG | | GGGGC | |
| | AGCGGAG | | CTGCG | |
| | | | GTCGG | |
| | | | A | |
| 19:52212718C > | CTCAGATGACACCCGCATGGTG | 117 | CGGCG | 1117 |
| G+ | CGGCGGGCCGCAGCCTCCAAG | | GGCCG | |
| | CTGGGG | | CAGCCT | |
| | | | CCAAG | |
| 19:52212729C > | CTCAGATGACACCCCCATGGTG | 118 | GGTGG | 1118 |
| T+ | CGGTGGGCCGCAGCCTCCAAG | | GCCGC | |
| | CTGGGGG | | AGCCTC | |
| | | | CAAGC | |
| 12:56085070G > | CATTGCCCAACCTCCGCATGGT | 119 | CGAGG | 1119 |
| A+ | GCGAGGGACCCAGGTCTACGA | | GACCC | |
| | TGGGAAG | | AGGTCT | |
| | | | ACGAT | |
| 17:7675139C > T− | CCGCGTCCaCGCCATGGCCATC | 120 | TACAA | 1120 |
| | TACAAGCAGTCACAGCACATG | | GCAGT | |
| | ACGGAG | | CACAG | |
| | | | CACAT | |
| | | | G | |
| 3:195782159T > | CTGAGGAAGTGCCGGTGACAG | 121 | GGAAG | 1121 |
| C− | GAAGAGCGGTGGCCTGACCTG | | AGCGG | |
| | TGGAT | | TGGCCT | |
| | | | GACCT | |
| 17:7675124T > C− | CTGTGACTGCTTGCAGATGGCC | 122 | TGGCG | 1122 |
| | ATGGCGCGGACGCGGGTGCCG | | CGGAC | |
| | GGCGGGG | | GCGGG | |
| | | | TGCCG | |
| | | | G | |
| 12:16040G > A− | CAGGGAAGTGGTtGACCCCTCC | 123 | CCGGT | 1123 |
| | GGTGGCTGGGCCACTCTGCTAG | | GGCTG | |
| | AGT | | GGCCA | |
| | | | CTCTGC | |
| 10:47461391G > | CAGAGAAGCGGGCGCGCGGGG | 124 | GGGCG | 1124 |
| A− | GCGGGCGCGtGGGGCCTTGCCG | | GGCGC | |
| | GAG | | GtGGGG | |
| | | | CCTTG | |
| 3:195783009C > | CACTGAGGAAGCGTCGGTGAC | 125 | GGAAG | 1125 |
| T− | AGGAAGAGAGGTGGCGTGACC | | AGAGG | |
| | TGTGGAT | | TGGCGT | |
| | | | GACCT | |
| 9:21971121G > A− | CCAGGAAGCCCTCCCGGGCAG | 126 | AGCGT | 1126 |
| | CGTCGTGCACGGGTCaGGTGAG | | CGTGC | |
| | AGT | | ACGGG | |
| | | | TCaGGT | |
| 17:7675139C > A− | CCGCGTCCtCGCCATGGCCATCT | 127 | ACAAG | 1127 |
| | ACAAGCAGTCACAGCACATGA | | CAGTC | |
| | CGGAGG | | ACAGC | |
| | | | ACATG | |
| | | | A | |
| 1:144436988C > | CTTGGAGGTCCTGCCCCTGGGA | 128 | GACTTG | 1128 |
| A− | CTTGTCCGGCTCATACGGAGTG | | TCCGGC | |
| | AGG | | TCATAC | |
| | | | GGA | |
| 17:7675139C > T− | CCGCGTCCaCGCCATGGCCATC | 129 | ACAAG | 1129 |
| | TACAAGCAGTCACAGCACATG | | CAGTC | |
| | ACGGAGG | | ACAGC | |
| | | | ACATG | |
| | | | A | |
| 7:55191822T > G+ | CACAGATTTTGGGCGGGCCAA | 130 | AACTG | 1130 |
| | ACTGCTGGGTGCGGAAGAGAA | | CTGGGT | |
| | AGAAT | | GCGGA | |
| | | | AGAGA | |
| 11:1097397C > T+ | CGGTGGGTaTTGGGGTTGGGGT | 131 | GTCACC | 1131 |
| | CACCGTAGTGGTGGTGGTGATG | | GTAGT | |
| | GGT | | GGTGG | |
| | | | TGGTG | |
| 19:52212718C > | CTCAGATGACACCCGCATGGTG | 132 | GGCGG | 1132 |
| G+ | CGGCGGGCCGCAGCCTCCAAG | | GCCGC | |
| | CTGGGGG | | AGCCTC | |
| | | | CAAGC | |
| 1:144436988C > | CTTGGAGGTCCTGCCCCTGGGA | 133 | TTGTCC | 1133 |
| A− | CTTGTCCGGCTCATACGGAGTG | | GGCTC | |
| | AGGAGG | | ATACG | |
| | | | GAGTG | |
| 19:52212729C > | CGGTGGGCCGCAGCCTCCAAG | 134 | CTGGG | 1134 |
| T+ | CTGGGGGAGTTTGCCAAGGTGC | | GGAGT | |
| | TGGAG | | TTGCCA | |
| | | | AGGTG | |
| 6:73519043A > G− | CTGAGCCACCCTACAGCCAGA | 135 | AGAGA | 1135 |
| | AGAGATATGAGGAAATcGTTAA | | TATGA | |
| | GGAAG | | GGAAA | |
| | | | TcGTTA | |
| 10:45826548G > | CTGGGTCTCACAGTCCACACAG | 136 | GTGGG | 1136 |
| A− | TGGGCATTCCCACGCATGTTTT | | CATTCC | |
| | GGAT | | CACGC | |
| | | | ATGTT | |
| 10:46131074C > | CGGTGGCCCaCAGCAGCTGCTG | 137 | TGGCCC | 1137 |
| T+ | GCCCAGGGACAGCTCAGTGCA | | AGGGA | |
| | GGGT | | CAGCTC | |
| | | | AGTG | |
| 12:56085070G > | CTGGGTCCCTCGCACCAtGCGG | 138 | AGGTT | 1138 |
| A+ | AGGTTGGGCAATGGTAGAGTA | | GGGCA | |
| | GAGAAT | | ATGGT | |
| | | | AGAGT | |
| | | | A | |
| 17:7675143C > A− | CTCCGTCATGTGCTGTGACTGC | 139 | TGCTTG | 1139 |
| | TTGTAGATGGCCATGGCGCGGA | | TAGAT | |
| | AG | | GGCCA | |
| | | | TGGCG | |
| 2:113595432G > | CGGGGGCGTCTCTGCAGGCCA | 140 | GGGTC | 1140 |
| A+ | GGGTCCTGGGCGCtCGTGAAGA | | CTGGG | |
| | TGGAG | | CGCtCG | |
| | | | TGAAG | |
| 7:55191822T > G+ | CGCAGCATGTCAAGATCACAG | 141 | GATTTT | 1141 |
| | ATTTTGGGCGGGCCAAACTGCT | | GGGCG | |
| | GGGT | | GGCCA | |
| | | | AACTG | |
| 17:7675124T > C− | CCGCGTCCGCGCCATGGCCATC | 142 | TgCAAG | 1142 |
| | TgCAAGCAGTCACAGCACATGA | | CAGTC | |
| | CGGAG | | ACAGC | |
| | | | ACATG | |
| 19:49665874C > | CGCTGGGCTGTTCCCGCCCCTA | 143 | GCCCTT | 1143 |
| T+ | TGCCCTTTTTTGGGTTTTCGGCC | | TTTTGG | |
| | AGAGG | | GTTTTC | |
| | | | GGC | |
| 17:7673579G > A− | CTTTGGCTAGGGAGAGGAGCT | 144 | TGTTGT | 1144 |
| | GGTGTTGTTGGGCAGTGCTAGG | | TGGGC | |
| | AAAGAGG | | AGTGCT | |
| | | | AGGA | |
| 12:17513A > C− | CAGCGGGTGTCAgCTTGCCTGA | 145 | CCCCCA | 1145 |
| | CCCCCATGTCGCCTCTGTAGGT | | TGTCGC | |
| | AGAAG | | CTCTGT | |
| | | | AGG | |
| 3:185152807G > | CCGCGCGGAGGGAGAGGAAAT | 146 | AGTGC | 1146 |
| A− | GAGTGCGGCGGCCTtGCCGGCG | | GGCGG | |
| | TCGGAG | | CCTtGC | |
| | | | CGGCG | |
| 22:21772875C > | CCCTGAAGCAGCAGCCAGGAA | 147 | CATGA | 1147 |
| T− | CATGAGCTCTTACCTTGTCACT | | GCTCTT | |
| | CGGGT | | ACCTTG | |
| | | | TCAC | |
| 17:7673802C > A− | CTTTGAGGTGCtTGTTTGTGCCT | 148 | GTCCTG | 1148 |
| | GTCCTGGGAGAGACCGGCGCA | | GGAGA | |
| | CAGAGG | | GACCG | |
| | | | GCGCA | |
| 17:7670685G > A− | CTTCGAGATGTTCtGAGAGCTG | 149 | CTGAAT | 1149 |
| | AATGAGGCCTTGGAACTCAAG | | GAGGC | |
| | GAT | | CTTGGA | |
| | | | ACTC | |
| 17:7675124T > C− | CCGCGTCCGCGCCATGGCCATC | 150 | gCAAGC | 1150 |
| | TgCAAGCAGTCACAGCACATGA | | AGTCA | |
| | CGGAGG | | CAGCA | |
| | | | CATGA | |
| 17:7675190C > T− | CACAGGGTAGGTCTTGGCCAGT | 151 | GGCAA | 1151 |
| | TGGCAAAACATCTTGTTGAGGG | | AACAT | |
| | CAGGGG | | CTTGTT | |
| | | | GAGGG | |
| 17:7673796C > T− | CTTTGAGGTGCGTGTTTaTGCCT | 152 | GTCCTG | 1152 |
| | GTCCTGGGAGAGACCGGCGCA | | GGAGA | |
| | CAGAGG | | GACCG | |
| | | | GCGCA | |
| 7:55165350G > T+ | CATTGACGGCCCCCACTGCGTC | 153 | AGACC | 1153 |
| | AAGACCTGCCCGGCAGTAGTC | | TGCCCG | |
| | ATGGGAG | | GCAGT | |
| | | | AGTCA | |
| 17:7673781C > G− | CTTTGAGGTGCGTGTTTGTGCC | 154 | GTCCTG | 1154 |
| | TGTCCTGGGAcAGACCGGCGCA | | GGACAG | |
| | CAGAGG | | ACCGG | |
| | | | CGCA | |
| 17:7673802C > T− | CTTTGAGGTGCaTGTTTGTGCCT | 155 | GTCCTG | 1155 |
| | GTCCTGGGAGAGACCGGCGCA | | GGAGA | |
| | CAGAGG | | GACCG | |
| | | | GCGCA | |
| 17:7673776G > A− | CTTTGAGGTGCGTGTTTGTGCC | 156 | GTCCTG | 1156 |
| | TGTCCTGGGAGAGACtGGCGCA | | GGAGA | |
| | CAGAGG | | GACtGG | |
| | | | CGCA | |
| 3:41224633A > G+ | CACTGCCACAGCTCCTTCTCTG | 157 | GTGGT | 1157 |
| | AGTGGTAAAGGCAATCCTGAG | | AAAGG | |
| | GAAGAGG | | CAATCC | |
| | | | TGAGG | |
| 11:1097434C > T+ | CAGTGGGTGTTGGGGTTGGGGT | 158 | GTCACC | 1158 |
| | CACCGTGGTGGTGGTGGTaATG | | GTGGT | |
| | GGT | | GGTGG | |
| | | | TGGTa | |
| 11:1097377T > C+ | CGGTGGGTGTTGGGGTTGGGGT | 159 | GTCACC | 1159 |
| | CACCGTgGTGGTGGTGGTGATG | | GTgGTG | |
| | GGT | | GTGGT | |
| | | | GGTG | |
| 20:58909365C > | CCTGGAACTTGGTCTCAAAGAT | 160 | TCCAG | 1160 |
| T+ | TCCAGAAGTCAGGACACaGCAG | | AAGTC | |
| | CGAAG | | AGGAC | |
| | | | ACaGCA | |
| 17:7673781C > T− | CTTTGAGGTGCGTGTTTGTGCC | 161 | GTCCTG | 1161 |
| | TGTCCTGGGAaAGACCGGCGCA | | GGAaAG | |
| | CAGAGG | | ACCGG | |
| | | | CGCA | |
| 17:7673806C > T− | CTTTGAGaTGCGTGTTTGTGCCT | 162 | GTCCTG | 1162 |
| | GTCCTGGGAGAGACCGGCGCA | | GGAGA | |
| | CAGAGG | | GACCG | |
| | | | GCGCA | |
| 3:41224633A > G+ | CACTGCCACAGCTCCTTCTCTG | 163 | TGAGT | 1163 |
| | AGTGGTAAAGGCAATCCTGAG | | GGTAA | |
| | GAAG | | AGGCA | |
| | | | ATCCTG | |
| 3:185152807G > | CGACGCCGGCAAGGCCGCCGC | 164 | ACTCAT | 1164 |
| A− | ACTCATTTCCTCTCCCTCCGCG | | TTCCTC | |
| | CGGAG | | TCCCTC | |
| | | | CGC | |
| 2:113595432G > | CCTGGGCGCtCGTGAAGATGGA | 165 | AGCCA | 1165 |
| A+ | GCCATATTCCTGCAGGCGCTCT | | TATTCC | |
| | GGAG | | TGCAG | |
| | | | GCGCT | |
| 10:121520163G > | CTTGGAGGATGGGCCGGTGAG | 166 | GCCATC | 1166 |
| C− | GCCATCGCTCTGGTGGAGAGA | | GCTCTG | |
| | GGGAAG | | GTGGA | |
| | | | GAGA | |
| 19:52212718C > | CCATGcGGGTGTCATCTGAGCA | 167 | GCACA | 1167 |
| G+ | CAGGTTCCGGAAGTACCTGGG | | GGTTCC | |
| | AGT | | GGAAG | |
| | | | TACCT | |
| 3:195782483C > | CGGGGTGGCGTGACCGGTGGA | 168 | TGTTGA | 1168 |
| T− | TGTTGAGGAAGGGCTGGTGAC | | GGAAG | |
| | ATGAAG | | GGCTG | |
| | | | GTGAC | |
| 3:185152807G > | CGGAGGGAGAGGAAATGAGTG | 169 | GGCGG | 1169 |
| A− | CGGCGGCCTtGCCGGCGTCGGA | | CCTtGC | |
| | GCGGGG | | CGGCG | |
| | | | TCGGA | |
| 12:56085070G > | CGTAGACCTGGGTCCCTCGCAC | 170 | CAtGCG | 1170 |
| A+ | CAtGCGGAGGTTGGGCAATGGT | | GAGGT | |
| | AGAGT | | TGGGC | |
| | | | AATGG | |
| 17:7673767C > T− | CTGGGAGAGACCGGCGCACAa | 171 | AGGAA | 1171 |
| | AGGAAGAGAATCTCCGCAAGA | | GAGAA | |
| | AAGGGG | | TCTCCG | |
| | | | CAAGA | |
| 17:7673767C > T− | CTGGGAGAGACCGGCGCACAa | 172 | GAAGA | 1172 |
| | AGGAAGAGAATCTCCGCAAGA | | GAATCT | |
| | AAGGGGAG | | CCGCA | |
| | | | AGAAA | |
| 17:7674216C > A− | CGGAGtCCCATCCTCACCATCA | 173 | TCACAC | 1173 |
| | TCACACTGGAAGACTCCAGGTC | | TGGAA | |
| | AGGAG | | GACTCC | |
| | | | AGGT | |
| 17:7673764C > T− | CTGGGAGAGACCGGCGCACAG | 174 | AGaAAG | 1174 |
| | AGaAAGAGAATCTCCGCAAGA | | AGAAT | |
| | AAGGGG | | CTCCGC | |
| | | | AAGA | |
| 10:46131074C > | CACTGAGCTGTCCCTGGGCCAG | 175 | AGCTG | 1175 |
| T+ | CAGCTGCTGTGGGCCACCGCTG | | CTGTGG | |
| | ATGAGG | | GCCAC | |
| | | | CGCTG | |
| 12:17743A > G− | CCTCGGCCCTGCCTTCTGGCCA | 176 | CCATAC | 1176 |
| | TACAGGTTCTCGGTGGTGTTGA | | AGGTTC | |
| | AG | | TCGGTG | |
| | | | GTG | |
| 17:7673764C > T− | CTGGGAGAGACCGGCGCACAG | 177 | aAAGAG | 1177 |
| | AGaAAGAGAATCTCCGCAAGA | | AATCTC | |
| | AAGGGGAG | | CGCAA | |
| | | | GAAA | |
| 3:185152807G > | CGGAGGGAGAGGAAATGAGTG | 178 | GCGGC | 1178 |
| A− | CGGCGGCCTtGCCGGCGTCGGA | | CTtGCC | |
| | GCGGGGT | | GGCGT | |
| | | | CGGAG | |
| 17:7673776G > A− | CTGGGAGAGACtGGCGCACAGA | 179 | AGGAA | 1179 |
| | GGAAGAGAATCTCCGCAAGAA | | GAGAA | |
| | AGGGG | | TCTCCG | |
| | | | CAAGA | |
| 17:7673704G > A− | CTAAGtGAGGTAAGCAAGCAGG | 180 | CAAGA | 1180 |
| | ACAAGAAGCGGTGGAGGAGAC | | AGCGG | |
| | CAAGGGT | | TGGAG | |
| | | | GAGAC | |
| | | | C | |
| 17:7674953T > C− | CGCTGAGGAGGGGCCAGACCT | 181 | TAAGA | 1181 |
| | AAGAGCAATCAGTGAGGAATC | | GCAAT | |
| | AGAGG | | CAGTG | |
| | | | AGGAA | |
| | | | T | |
| 17:7673776G > A− | CTGGGAGAGACtGGCGCACAGA | 182 | GAAGA | 1182 |
| | GGAAGAGAATCTCCGCAAGAA | | GAATCT | |
| | AGGGGAG | | CCGCA | |
| | | | AGAAA | |
| 3:41224622C > T+ | CTCAGAGAAGGAGCTGTGGTA | 183 | TAGTG | 1183 |
| | GTGGCACCAaAATGGATTCCAG | | GCACC | |
| | AGT | | AaAATG | |
| | | | GATTC | |
| 3:41224645T > C+ | CTCAGAGgAGGAGCTGTGGTAG | 184 | TAGTG | 1184 |
| | TGGCACCAGAATGGATTCCAG | | GCACC | |
| | AGT | | AGAAT | |
| | | | GGATTC | |
| 17:39725079G > | CCCTGTGTAtGAGCCGCACATC | 185 | TCCTCC | 1185 |
| A+ | CTCCAGGTAGCTCATCCCCTGG | | AGGTA | |
| | AAT | | GCTCAT | |
| | | | CCCC | |
| 17:7673704G > A− | CCCAGGGAGCACTAAGtGAGGT | 186 | AAGCA | 1186 |
| | AAGCAAGCAGGACAAGAAGCG | | AGCAG | |
| | GTGGAG | | GACAA | |
| | | | GAAGC | |
| | | | G | |
| 3:41224633A > G+ | CTCAGAGAAGGAGCTGTGGcAG | 187 | CAGTGG | 1187 |
| | TGGCACCAGAATGGATTCCAG | | CACCA | |
| | AGT | | GAATG | |
| | | | GATTC | |
| 7:56106490G > A− | CGAAGAGCTAAGCGACTTCTG | 188 | GAGGA | 1188 |
| | AGGAGACCGGAAAATGGGAGG | | GACCG | |
| | CGGGG | | GAAAA | |
| | | | TGGGA | |
| | | | G | |
| 16:15104542T > | CGCTGAGGGTGGAGCTGAGGG | 189 | GAAGG | 1189 |
| A− | TGGAAGGGGAGTGAGCAGACA | | GGAGT | |
| | CACGGAGG | | GAGCA | |
| | | | GACAC | |
| | | | A | |
| 3:185152807G > | CGCGGAGGGAGAGGAAATGAG | 190 | AGTGC | 1190 |
| A− | TGCGGCGGCCTtGCCGGCGTCG | | GGCGG | |
| | GAG | | CCTtGC | |
| | | | CGGCG | |
| 5:112838934C > | CGGGGAGCCAATGGTTCAGAA | 191 | AAATT | 1191 |
| T+ | ACAAATTGAGTGGGTTCTAATC | | GAGTG | |
| | ATGGAAT | | GGTTCT | |
| | | | AATCA | |
| 16:15104542T > | CGCTGAGGGTGGAGCTGAGGG | 192 | GGAAG | 1192 |
| A− | TGGAAGGGGAGTGAGCAGACA | | GGGAG | |
| | CACGGAG | | TGAGC | |
| | | | AGACA | |
| | | | C | |
| 17:7673806C > T− | CTGGGACGGAACAGCTTTGAGa | 193 | GaTGCG | 1193 |
| | TGCGTGTTTGTGCCTGTCCTGG | | TGTTTG | |
| | GAG | | TGCCTG | |
| | | | TCC | |
| 17:7673803G > A− | CTGGGACGGAACAGCTTTGAG | 194 | GGTGtG | 1194 |
| | GTGtGTGTTTGTGCCTGTCCTGG | | TGTTTG | |
| | GAG | | TGCCTG | |
| | | | TCC | |
| 17:7673802C > A− | CTGGGACGGAACAGCTTTGAG | 195 | GGTGCt | 1195 |
| | GTGCtTGTTTGTGCCTGTCCTGG | | TGTTTG | |
| | GAG | | TGCCTG | |
| | | | TCC | |
| 17:7673802C > T− | CTGGGACGGAACAGCTTTGAG | 196 | GGTGCa | 1196 |
| | GTGCaTGTTTGTGCCTGTCCTGG | | TGTTTG | |
| | GAG | | TGCCTG | |
| | | | TCC | |
| 17:7673796C > T− | CTGGGACGGAACAGCTTTGAG | 197 | GGTGC | 1197 |
| | GTGCGTGTTTaTGCCTGTCCTGG | | GTGTTT | |
| | GAG | | aTGCCT | |
| | | | GTCC | |
| 3:195779688A > | CGTGGCCTGTGGATACTGAGGA | 198 | GGAAG | 1198 |
| G− | AGTGTCGGTGACAGGAAGAGG | | TGTCGG | |
| | GGT | | TGACA | |
| | | | GGAAG | |
| 10:121520163G > | CGGTGAGGCCATCGCTCTGGTG | 199 | GAGAG | 1199 |
| C− | GAGAGAGGGAAGAAAGGAGG | | AGGGA | |
| | AGTGGGG | | AGAAA | |
| | | | GGAGG | |
| | | | A | |
| 16:15104542T > | CTGAGGGTGGAGCTGAGGGTG | 200 | GGAAG | 1200 |
| A− | GAAGGGGAGTGAGCAGACACA | | GGGAG | |
| | CGGAG | | TGAGC | |
| | | | AGACA | |
| | | | C | |
| 16:15104542T > | CTGAGGGTGGAGCTGAGGGTG | 201 | GAAGG | 1201 |
| A− | GAAGGGGAGTGAGCAGACACA | | GGAGT | |
| | CGGAGG | | GAGCA | |
| | | | GACAC | |
| | | | A | |
| 3:179218304A > | CTGTGACTCCATAGAAAATCTT | 202 | CTCCTG | 1202 |
| C+ | TCTCCTGCgCAGTGATTTCAGA | | CgCAGT | |
| | GAGAGG | | GATTTC | |
| | | | AGA | |
| 17:7673704G > A− | CAGGGAGCACTAAGtGAGGTAA | 203 | AGCAA | 1203 |
| | GCAAGCAGGACAAGAAGCGGT | | GCAGG | |
| | GGAGG | | ACAAG | |
| | | | AAGCG | |
| | | | G | |
| 3:179218303G > | CTGTGACTCCATAGAAAATCTT | 204 | CTCCTG | 1204 |
| A+ | TCTCCTGCTtAGTGATTTCAGAG | | CTtAGT | |
| | AGAGG | | GATTTC | |
| | | | AGA | |
| 17:7673704G > A− | CAGGGAGCACTAAGGAGGTAA | 205 | CAAGC | 1205 |
| | GCAAGCAGGACAAGAAGCGGT | | AGGAC | |
| | GGAGGAG | | AAGAA | |
| | | | GCGGT | |
| | | | G | |
| 9:21971121G > A− | CGCCGACCCCGCCACTCTCACC | 206 | GACCC | 1206 |
| | tGACCCGTGCACGACGCTGCCC | | GTGCA | |
| | GGGAGG | | CGACG | |
| | | | CTGCCC | |
| 2:177234217G > | CTTGGAGTAAGTgGAGAAGTAT | 207 | TTGACT | 1207 |
| C− | TTGACTTCAGTCAGCGACGGAA | | TCAGTC | |
| | AGAGT | | AGCGA | |
| | | | CGGA | |
| 17:7673704G > A− | CAGGGAGCACTAAGtGAGGTAA | 208 | AAGCA | 1208 |
| | GCAAGCAGGACAAGAAGCGGT | | AGCAG | |
| | GGAG | | GACAA | |
| | | | GAAGC | |
| | | | G | |
| 3:179218307A > | CTGTGACTCCATAGAAAATCTT | 209 | CTCCcG | 1209 |
| G+ | TCTCCcGCTCAGTGATTTCAGA | | CTCAGT | |
| | GAGAGG | | GATTTC | |
| | | | AGA | |
| 3:179218294G > | CTGTGACTCCATAGAAAATCTT | 210 | CTCCTG | 1210 |
| A+ | TCTCCTGCTCAGTGATTTtAGAG | | CTCAGT | |
| | AGAGG | | GATTTt | |
| | | | AGA | |
| 17:7673803G > A− | CTTTGAGGTGtGTGTTTGTGCCT | 211 | GTCCTG | 1211 |
| | GTCCTGGGAGAGACCGGCGCA | | GGAGA | |
| | CAGAGG | | GACCG | |
| | | | GCGCA | |
| 17:7673704G > A− | CCCAGGGAGCACTAAGtGAGGT | 212 | AGCAA | 1212 |
| | AAGCAAGCAGGACAAGAAGCG | | GCAGG | |
| | GTGGAGG | | ACAAG | |
| | | | AAGCG | |
| | | | G | |
| 12:16040G > A− | CGGAGGGGTCAACCACTTCCCT | 213 | GGAGC | 1213 |
| | GGGAGCTCCCTGGACTGGAGC | | TCCCTG | |
| | CGGGAGG | | GACTG | |
| | | | GAGCC | |
| 19:3118944A > T+ | CGCTGTGTCCTTTCAGGATGGT | 214 | GTGGA | 1214 |
| | GGATGTGGGGGGCCTGCGGTC | | TGTGG | |
| | GGAG | | GGGGC | |
| | | | CTGCG | |
| | | | G | |
| 6:73519043A > G− | CACTGAGCCACCCTACAGCCAG | 215 | AGAGA | 1215 |
| | AAGAGATATGAGGAAATcGTTA | | TATGA | |
| | AGGAAG | | GGAAA | |
| | | | TcGTTA | |
| 17:7675994C > A− | CTGGGACAGCCAAGTCTGTGAC | 216 | ACTTGC | 1216 |
| | TTGCACtGTCAGTTGCCCTGAG | | ACtGTC | |
| | GGG | | AGTTGC | |
| | | | CCT | |
| 12:16040G > A− | CGGAGGGGTCAACCACTTCCCT | 217 | GGGAG | 1217 |
| | GGGAGCTCCCTGGACTGGAGC | | CTCCCT | |
| | CGGGAG | | GGACT | |
| | | | GGAGC | |
| 16:15104542T > | CACGGAGGTGTCTTGAGATTAT | 218 | TATCAT | 1218 |
| A− | CATCCGCTGAGGGTGGAAGGG | | CCGCTG | |
| | GAT | | AGGGT | |
| | | | GGAA | |
| 9:21971121 G >A− | CGCCGACCCCGCCACTCTCACC | 219 | tGACCC | 1219 |
| | tGACCCGTGCACGACGCTGCCC | | GTGCA | |
| | GGGAG | | CGACG | |
| | | | CTGCC | |

Deletions

| | | | | |
|---|---|---|---|---|
| 10:12829237del | CTATGGTGTGTTTTTTCATTAAT | 220 | GACAA | 1220 |
| A+ | GACAAAAAAAAAAGGTTTCAA | | AAAAA | |
| | CTGGAT | | AAAGG | |
| | | | TTTCAA | |
| 2:101891433del | CATTGCAGCCTTGATTTATTTT | 221 | GAGTA | 1221 |
| T+ | GGAGTAAGAAAAAAAAAAGAA | | AGAAA | |
| | TGGGGAT | | AAAAA | |
| | | | AAGAA | |
| | | | T | |
| 3:18348736delT− | CTTTGGAAACTTGCCCCTTATT | 222 | TTTAAA | 1222 |
| | TAAAAAAAAAAAGAAAAAAAA | | AAAAA | |
| | GAGT | | AAAGA | |
| | | | AAAAA | |
| 2:101891433del | CATTGCAGCCTTGATTTATTTT | 223 | TGGAG | 1223 |
| T+ | GGAGTAAGAAAAAAAAAAGAA | | TAAGA | |
| | TGGGG | | AAAAA | |
| | | | AAAAG | |
| | | | A | |
| 1:12725526delT+ | CACTGCTCACCTGGAGTTTCAT | 224 | CTGCTA | 1224 |
| | CTGCTACTTTTTTTTCAAAACCT | | CTTTTT | |
| | GGAT | | TTTCAA | |
| | | | AAC | |
| 11:64204409delT+ | CATAGTTGGTTTTTTTTTATTTG | 225 | TGGGG | 1225 |
| | GGGCAGTGGGCATGTTATGGG | | CAGTG | |
| | GAGG | | GGCAT | |
| | | | GTTATG | |
| 14:41904405del | CAATGTAGGCTTTTTTTCTTTTT | 226 | TAAAA | 1226 |
| T+ | TAAAAAAAAATACTTGTAGGC | | AAAAA | |
| | CAGAAG | | TACTTG | |
| | | | TAGGC | |
| 11:64204409del | CATAGTTGGTTTTTTTTTATTTG | 227 | GGGCA | 1227 |
| T+ | GGGCAGTGGGCATGTTATGGG | | GTGGG | |
| | GAGGGG | | CATGTT | |
| | | | ATGGG | |
| 14:55684263del | CAGAGTTGGCTTTTTTTTTCCTT | 228 | TCTTTA | 1228 |
| A+ | CTTTAAATCAGTAGTCTAACAA | | AATCA | |
| | GGAT | | GTAGTC | |
| | | | TAAC | |
| 14:20992723del | CAGCGGAGATCCTCTTTAAAAA | 229 | AAAAT | 1229 |
| T+ | AAAATGCATATGCAATGTCCCA | | GCATAT | |
| | AGAAT | | GCAAT | |
| | | | GTCCC | |
| 12:121238563del | CTTTGGTGTTTCTTCATTAAAA | 230 | AGAAA | 1230 |
| A− | GAAAAAAAAAAGTCATCAATG | | AAAAA | |
| | TGGGT | | AAGTC | |
| | | | ATCAAT | |
| 10:100827567del | CTGTGTTAACTTCCAGGTTCCC | 231 | CTTATT | 1231 |
| C+ | CTTATTATTATAGTGCCGCCCC | | ATTATA | |
| | CGGGG | | GTGCC | |
| | | | GCCC | |
| 11:64204409del | CATAGTTGGTTTTTTTTTATTTG | 232 | TTGGG | 1232 |
| T+ | GGGCAGTGGGCATGTTATGGG | | GCAGT | |
| | GAG | | GGGCA | |
| | | | TGTTAT | |
| 5:134727810del | CAATGGTCTGCATGGGTTACAA | 233 | ATGACT | 1233 |
| A+ | ATGACTTTTTTTTTTTTTAACAG | | TTTTTT | |
| | GAAT | | TTTTTT | |
| | | | AAC | |
| 6:17615262delT− | CAAAGTTGCCTAAATCCATTTG | 234 | GGAAA | 1234 |
| | GAAATCTTTAAAAAAAAATTG | | TCTTTA | |
| | GGGAT | | AAAAA | |
| | | | AAATT | |
| 8:115411283del | CAATGCTAGTCGTTACTACTAT | 235 | TCTGTC | 1235 |
| A− | GTCTGTCTGAGAAAAAAAAAA | | TGAGA | |
| | ATTGAAT | | AAAAA | |
| | | | AAAAA | |
| 4:157221000del | CAGAGGAAAACAGCCAAAGAA | 236 | AAGAG | 1236 |
| A+ | GGAAGAGGAGGAAAAGGAAA | | GAGGA | |
| | AAAAAGGGG | | AAAGG | |
| | | | AAAAA | |
| | | | A | |
| X:108439959del | CTTGGGTGAAGAGAAAGAAGC | 237 | TTTAAG | 1237 |
| T+ | TTTTTAAGAGTGGAAGAAAAA | | AGTGG | |
| | AAAAGAAG | | AAGAA | |
| | | | AAAAA | |
| 11:129882601del | CTTGGCTGTCCTTAAAAAAAGG | 238 | GTTAA | 1238 |
| T− | TTAAGGAAAAAGAGGAAAAGA | | GGAAA | |
| | AGAAG | | AAGAG | |
| | | | GAAAA | |
| | | | G | |
| 7:44080815delG− | CTCTGTTAACCAGCTTATGTCC | 239 | AGCAG | 1239 |
| | AGCAGAGCTGGGGGGTGCAAC | | AGCTG | |
| | CCGGGG | | GGGGG | |
| | | | TGCAA | |
| | | | C | |
| 1:26780313delC+ | CAATGTGGACCTGATTCTGGCC | 240 | CACAC | 1240 |
| | ACACCCCCTTCAGCCGCCTGGA | | CCCCTT | |
| | GAAG | | CAGCC | |
| | | | GCCTG | |
| 10:26173831del | CTGTGGAGAGTAACAACAGAG | 241 | AGTGT | 1241 |
| A+ | TGTATCAGACTCCAAAAAAATG | | ATCAG | |
| | AAT | | ACTCCA | |
| | | | AAAAA | |
| 10:46809223del | CATGGGTTGTTTATTCTCGCTCT | 242 | TCTCTC | 1242 |
| T− | CTCTCTTTTTTTTTTTTTGAGAG | | TCTTTT | |
| | G | | TTTTTT | |
| | | | TTT | |
| 10:46809223del | CATGGGTTGTTTATTCTCGCTCT | 243 | CTCTCT | 1243 |
| T− | CTCTCTTTTTTTTTTTTTGAGAG | | TTTTTT | |
| | GGAG | | TTTTTT | |
| | | | GAG | |
| 12:12121051del | CAACGTAAAAATGTAAATATA | 244 | ATTTGG | 1244 |
| C− | AATTTGGTTGAGATCTGGAGGG | | TTGAG | |
| | GGGAGG | | ATCTGG | |
| | | | AGGG | |
| 3:185644316del | CTTGGTAAATTTCTACTTTCCTC | 245 | CACATT | 1245 |
| T− | CACATTTTTTTTTTTAAAGAAA | | TTTTTT | |
| | GGAAT | | TTTAAA | |
| | | | GAA | |
| 13:26214104del | CCTTGCTCACTTTTTTTACTAAA | 246 | AAATG | 1246 |
| A− | TGAAAGTGATGATGATGATCG | | AAAGT | |
| | AAT | | GATGA | |
| | | | TGATG | |
| | | | A | |
| 5:159203632del | CTTTGCAGCCATGTTGTTTTTTT | 247 | TTTTTC | 1247 |
| TC− | TTTCTTTTTTTTTTTCTTGGAGA | | TTTTTT | |
| | AG | | TTTTTC | |
| | | | TTG | |
| 1:45014295delT+ | CTGAGGAAAGTAAATTTTTTTT | 248 | TTTTTT | 1248 |
| | TTTTAATTACTGGGTTTTTAGG | | TAATTA | |
| | GT | | CTGGGT | |
| | | | TTT | |
| 9:34088276delA− | CTCAGTTTCCTTGTTTCTTTTGA | 249 | TGATTT | 1249 |
| | TTTTTTTTTCCTAATTGTGTGAG | | TTTTTT | |
| | G | | CCTAAT | |
| | | | TGT | |
| 4:144738016del | CACAGGAGTGTGTAGCAGGAT | 250 | AACAG | 1250 |
| A+ | AACAGTCTTTTTTTTTAATGAC | | TCTTTT | |
| | AGGAT | | TTTTTA | |
| | | | ATGA | |
| 2:161992536del | CATTGTAAATGTGCTTTTAAAA | 251 | AAATA | 1251 |
| T− | AAAATACTGATGTTCCTAGTGA | | CTGATG | |
| | AAGAGG | | TTCCTA | |
| | | | GTGA | |
| X:47635578delA− | CACTGTTTGTTTTACTTCCCCAA | 252 | AAATG | 1252 |
| | AATGGACCTTTTTTTTTCTAAA | | GACCTT | |
| | GAGT | | TTTTTT | |
| | | | TCTA | |
| 6:17615262delT− | CAAAGTTGCCTAAATCCATTTG | 253 | TTGGA | 1253 |
| | GAAATCTTTAAAAAAAAATTG | | AATCTT | |
| | GGG | | TAAAA | |
| | | | AAAAA | |
| 7:44080815delG− | CTCTGTTAACCAGCTTATGTCC | 254 | GCAGA | 1254 |
| | AGCAGAGCTGGGGGGTGCAAC | | GCTGG | |
| | CCGGGGG | | GGGGT | |
| | | | GCAAC | |
| | | | C | |
| 10:11331605del | CTTTGTAACCTTTTTTTGTTTTG | 255 | TTTGTT | 1255 |
| A+ | TTTTTTTTTTAAATATTAGGGAT | | TTTTTT | |
| | | | TTAAAT | |
| | | | ATT | |
| 4:56395207delA− | CCATGCTTATGTTTATAAGTTTT | 256 | TGAGA | 1256 |
| | GAGATTTTTTTTTTTCTGAAAA | | TTTTTT | |
| | GGAT | | TTTTTC | |
| | | | TGAA | |
| 11:30012016del | CAAAGCTGGGGCGGTTCCTGTC | 257 | TCAAA | 1257 |
| A− | AAAAAATACTCATTGCGCAAA | | AAATA | |
| | GGGT | | CTCATT | |
| | | | GCGCA | |
| 10:79920900del | CGGGGGTGAACAGGAATGGAG | 258 | TTGAGC | 1258 |
| A+ | CATTGAGCTTTTGGGGAAAAAA | | TTTTGG | |
| | AAAGAGT | | GGAAA | |
| | | | AAAA | |
| 20:4727794delT+ | CAATGCTTTCTACTCATTTTTCT | 259 | TTCTAT | 1259 |
| | ATACTTTTTTTTTGAGGCAGAG | | ACTTTT | |
| | T | | TTTTTG | |
| | | | AGG | |
| 8:61499736delT+ | CAAAGTTGGACAAAGACTTGA | 260 | GATGCT | 1260 |
| | GAGATGCTTTTTTTTCCCCCAG | | TTTTTT | |
| | TGAGGGG | | TCCCCC | |
| | | | AGT | |
| 8:61499736delT+ | CAAAGTTGGACAAAGACTTGA | 261 | GAGAT | 1261 |
| | GAGATGCTTTTTTTTCCCCCAG | | GCTTTT | |
| | TGAGG | | TTTTCC | |
| | | | CCCA | |
| 5:95793485delA+ | CTACGTAATTTTTTTTTTTAATC | 262 | TCTAAG | 1262 |
| | TAAGCATTTCTTAACTGAGAGG | | CATTTC | |
| | GGT | | TTAACT | |
| | | | GAG | |
| 15:44711583del | CTGTGCTCGCGCTATCTCTCTTT | 263 | CTGGCC | 1263 |
| CT+ | CTGGCCTGGAGGCTATCCAGCG | | TGGAG | |
| | TGAGT | | GCTATC | |
| | | | CAGC | |
| 5:95793485delA+ | CTACGTAATTTTTTTTTTTAATC | 264 | ATCTAA | 1264 |
| | TAAGCATTTCTTAACTGAGAGG | | GCATTT | |
| | GG | | CTTAAC | |
| | | | TGA | |
| 11:47175302del | CGCTGCTCTCTTCTTTGCTCTCC | 265 | CAGAC | 1265 |
| A− | AGACGGCTTTTTTCGCCAACAT | | GGCTTT | |
| | GGAT | | TTTCGC | |
| | | | CAAC | |
| 19:35732101del | CAGGGGAGGGCAGGGGGCCAA | 266 | GGAGA | 1266 |
| G+ | AGGAGACACCCCCAAGGGCCT | | CACCCC | |
| | CCGGGAT | | CAAGG | |
| | | | GCCTC | |
| 1:66924743delT− | CGGAGGAGCTTGGGGGTACAG | 267 | GACTTC | 1267 |
| | GGACTTCAGAGGCGGCCAAAA | | AGAGG | |
| | AAGGGGT | | CGGCC | |
| | | | AAAAA | |
| 12:12121051del | CAACGTAAAAATGTAAATATA | 268 | AATTTG | 1268 |
| C− | AATTTGGTTGAGATCTGGAGGG | | GTTGA | |
| | GGGAG | | GATCTG | |
| | | | GAGG | |
| 9:32633586delT− | CTTTGCTAAAGCACATCAAAAA | 269 | AAGGC | 1269 |
| | AAGGCCAAGATGAGAGAACAA | | CAAGA | |
| | GAGAGG | | TGAGA | |
| | | | GAACA | |
| | | | A | |
| 1:147259627del | CAAGGGATTTTTTTTTTAACAA | 270 | ATGGG | 1270 |
| A+ | AATGGGAAAGACCTATGCAGA | | AAAGA | |
| | AAGGAAG | | CCTATG | |
| | | | CAGAA | |
| 7:20786861delT− | CAGAGGATCTTTTTTATATTGA | 271 | AAATC | 1271 |
| | TAAATCAGAGGCAGTGTTTTTT | | AGAGG | |
| | TAGAGG | | CAGTGT | |
| | | | TTTTT | |
| 1:16135704delG− | CTCGGCTCTGCTGCGGCGGGGG | 272 | GGATG | 1272 |
| | ATGCTCCAGGAGACGCTAAGC | | CTCCAG | |
| | GAGG | | GAGAC | |
| | | | GCTAA | |
| 7:55174772delG | CGGAGATGTTTTGATAGCGACG | 273 | AATTTT | 1273 |
| GAATTAAGAG | GGAATTTTAACTTTCTCACCTT | | AACTTT | |
| AAGC+ | CTGGGAT | | CTCACC | |
| | | | TTC | |
| 19:35719862del | CCTAGCAGATGTGGCTCCTACC | 274 | CCCAA | 1274 |
| C+ | CCCCAAAGACCCCTGCCCGGA | | AGACC | |
| | AACGGGG | | CCTGCC | |
| | | | CGGAA | |
| 5:53884973delA− | CTATGTATCCTTCCTGATTCAT | 275 | GACATT | 1275 |
| | GACATTAAAAAAAAAAGCTTA | | AAAAA | |
| | AAGAAG | | AAAAA | |
| | | | GCTTA | |
| 12:49040709del | CTGGGCAGGGGTGGCTCCTGG | 276 | CCTTAG | 1276 |
| G− | GGCCTTAGGCCCAAGCCCGGG | | GCCCA | |
| | CTCTGGGG | | AGCCC | |
| | | | GGGCT | |
| 1:66924743delT− | CGGAGGAGCTTGGGGGTACAG | 277 | GGACTT | 1277 |
| | GGACTTCAGAGGCGGCCAAAA | | CAGAG | |
| | AAGGGG | | GCGGC | |
| | | | CAAAA | |
| X:129805329del | CGAGGCTTAATGGAAGAACTG | 278 | TGGTTA | 1278 |
| T− | GTTAGCATTTTTTTTTTTTGAGG | | GCATTT | |
| | GT | | TTTTTT | |
| | | | TTT | |
| 19:35719862del | CAGGGGTCTTTGGGGGGTAGG | 279 | AGCCA | 1279 |
| C+ | AGCCACATCTGCTAGGCGAGG | | CATCTG | |
| | AGGAGG | | CTAGG | |
| | | | CGAGG | |
| 11:62882057del | CCAAGGAAGATTTTGACAGTCT | 280 | TTGCAA | 1280 |
| A+ | CTTGCAATCGGCTAAAAAAAG | | TCGGCT | |
| | AGTGGGT | | AAAAA | |
| | | | AAGA | |
| 2:147899472del | CTCTGCTTATTTATAGGACTGA | 281 | TGTGTA | 1281 |
| A+ | TTGTGTAGAAAAAAAGACAGC | | GAAAA | |
| | CCTGAAG | | AAAGA | |
| | | | CAGCC | |
| 17:50356606del | CGTAGGTGTTCTCCCAGTAGGC | 282 | GCCTTG | 1282 |
| C+ | CTTGAGGGCTGGCGTGCCGGG | | AGGGC | |
| | GGGT | | TGGCGT | |
| | | | GCCG | |
| 10:844999delT− | CCTAGATAAGCAGAATTCCAAT | 283 | AATGTT | 1283 |
| | GTTTTTTAAGTACTTCTCGGGG | | TTTTAA | |
| | GT | | GTACTT | |
| | | | CTC | |
| 5:135334913del | CCCAGTAGGAGTGAAGGGGAT | 284 | TTTTTT | 1284 |
| T− | TTTTTTTTCTTTTAAACTGAAGG | | TCTTTT | |
| | TGGGG | | AAACT | |
| | | | GAAG | |
| 11:18210242del | CTCAGCAATGTCTTTTTTTTTCG | 285 | TTCGGC | 1285 |
| T+ | GCCTTCGAGGTCACGTTAAGGA | | CTTCGA | |
| | G | | GGTCA | |
| | | | CGTT | |
| 2:1649188delG− | CCCTGCTTCTCTGTCATGATCC | 286 | TCCCCC | 1286 |
| | CCCCAATGACTCCCGGGCCAGG | | CAATG | |
| | AG | | ACTCCC | |
| | | | GGGC | |
| 1:157697170del | CTCTGTACACGTATCTGAGATC | 287 | TCAGG | 1287 |
| T− | TCAGGCTCCTTTTTTGATGCTGT | | CTCCTT | |
| | GAGT | | TTTTGA | |
| | | | TGCT | |
| 7:100345930del | CTGAGTTAAAATGTGAAGGGA | 288 | GATTTT | 1288 |
| T− | TTTTTTTTTTCAGATTACTGAGA | | TTTTTT | |
| | GT | | CAGATT | |
| | | | ACT | |
| 15:75207463del | CTGTGGAAGAGACTGGCACTCC | 289 | GGGCA | 1289 |
| C+ | CGGGCACAGGGGGGCAGTGCT | | CAGGG | |
| | GGGGGGT | | GGGCA | |
| | | | GTGCTG | |
| X:80444260delT+ | CCCTGGACTTTTCAAGCATTTT | 290 | TTTTTT | 1290 |
| | TTTTGACAATTAAATTGGGTTG | | GACAA | |
| | GAT | | TTAAAT | |
| | | | TGGG | |
| 22:16590897del | CAAAGAAACACCCACCTCCTGT | 291 | GGAAA | 1291 |
| T− | GGAAACAAAAAAATCCTTGGA | | CAAAA | |
| | TTGAAT | | AAATC | |
| | | | CTTGGA | |
| 19:35719862del | CAGGGGTCTTTGGGGGGTAGG | 292 | GAGCC | 1292 |
| C+ | AGCCACATCTGCTAGGCGAGG | | ACATCT | |
| | AGGAG | | GCTAG | |
| | | | GCGAG | |
| 11:105003284del | CTGTGTAAAAAAATCATGATGA | 293 | GTGCTG | 1293 |
| T− | GGTGCTGTGCTCTCTGTATGAA | | TGCTCT | |
| | ATGGGG | | CTGTAT | |
| | | | GAA | |
| 4:128948520del | CAAGGCCAAATTGAACGAGTC | 294 | ATTAA | 1294 |
| T− | ATTAAGGAAAAAAAGCAGTGG | | GGAAA | |
| | AAGAAG | | AAAAG | |
| | | | CAGTG | |
| | | | G | |
| 10:29471187del | CCAGGGAAAGGAGCCCCCCTG | 295 | GTTTCC | 1295 |
| C− | TTTCCTGCAGTGTTTCCAGGGG | | TGCAGT | |
| | GGAT | | GTTTCC | |
| | | | AGG | |
| 5:177248268del | CCCGGGATGCCTGCCTCTAAAA | 296 | AAATG | 1296 |
| A+ | AATGCAGGGTGAACGCGGTGG | | CAGGG | |
| | AGGAG | | TGAAC | |
| | | | GCGGT | |
| | | | G | |
| 10:8069470delC | CACAGGACGTCCCTGCTCTCCT | 297 | GGCTG | 1297 |
| A+ | GGCTGCAGACTAGAGTGGGGA | | CAGAC | |
| | GAGAGG | | TAGAG | |
| | | | TGGGG | |
| | | | A | |
| 12:48980565del | CAATGTTGTCGCTGCAGCCCCC | 298 | CCCCA | 1298 |
| G+ | CAGTGCCAGTCGGGGCCCCCG | | GTGCC | |
| | GGG | | AGTCG | |
| | | | GGGCC | |
| | | | C | |
| 8:10726136delG− | CAGGGGTGGCTACAGTGGAGA | 299 | GAGGG | 1299 |
| | GGGCTTGGGGCGTACTCCGGTG | | CTTGGG | |
| | AGT | | GCGTA | |
| | | | CTCCG | |
| 2:46187716delA+ | CAAAGGTGAAAAAACAATGCA | 300 | CATTCT | 1300 |
| | TTCTTGCTTTAAAAAAAAAAAG | | TGCTTT | |
| | AAG | | AAAAA | |
| | | | AAAA | |
| 10:8069470delC | CCCTGCTCTCCTGGCTGCAGAC | 301 | GACTA | 1301 |
| A+ | TAGAGTGGGGAGAGAGGAGAG | | GAGTG | |
| | GGT | | GGGAG | |
| | | | AGAGG | |
| | | | A | |
| 12:6602380delT− | CTCGGGACCCTAAAATCCCTAA | 302 | GAGCA | 1302 |
| | GAGCAAGCGCCAAAAAAGGAG | | AGCGC | |
| | GTGAGT | | CAAAA | |
| | | | AAGGA | |
| | | | G | |
| X:133027177del | CACGGCTACTTTTTTTAAAGAT | 303 | GATAC | 1303 |
| A− | ACCTATAATATAGAAATCAAG | | CTATAA | |
| | GAG | | TATAG | |
| | | | AAATC | |
| 1:155317840del | CAGAGCTGCTACTTTATATCTG | 304 | ATATA | 1304 |
| A+ | TATATAGTTTTGCTTTTTTTGGT | | GTTTTG | |
| | AGGGG | | CTTTTT | |
| | | | TTGG | |
| 5:14711100delA− | CATCGTATTTTGTTCCCTTTTTT | 305 | TTTGTT | 1305 |
| | TGTTTTGTTTTGGTAATGAAAG | | TTGTTT | |
| | AGG | | TGGTA | |
| | | | ATGA | |
| 16:88624733del | CTCGGGAAGCCAGGAGGAAGG | 306 | GCGGC | 1306 |
| C+ | AGCGGCCAGCCAGGACCCCCC | | CAGCC | |
| | CAGGAGG | | AGGAC | |
| | | | CCCCCC | |
| 10:21518000del | CAGTGTAATTTCATGGGGTTTC | 307 | CCCCCC | 1307 |
| G− | CCCCCCCCAATAATTTCGCCTA | | CCCAAT | |
| | GAGT | | AATTTC | |
| | | | GCC | |
| 18:36625553del | CACTGGAACACACGCTGGCCCT | 308 | CCTCCG | 1308 |
| C+ | CCTCCGGTCCCGGCACCCACTG | | GTCCCG | |
| | GGGGT | | GCACC | |
| | | | CACT | |
| 5:53884973delA− | CTTTGAAACATCAAACACTTCT | 309 | TAAGCT | 1309 |
| | TTAAGCTTTTTTTTTTAATGTCA | | TTTTTT | |
| | TGAAT | | TTTAAT | |
| | | | GTC | |
| 20:37999211del | CTAAGCACTGCCAGGGGGCGT | 310 | CCCGTG | 1310 |
| G− | CCCGTGTGAGTCGGTGAACGA | | TGAGTC | |
| | GCGAGG | | GGTGA | |
| | | | ACGA | |
| 1:16135704delG− | CCTCGCTTAGCGTCTCCTGGAG | 311 | GCATCC | 1311 |
| | CATCCCCCGCCGCAGCAGAGCC | | CCCGCC | |
| | GAGT | | GCAGC | |
| | | | AGAG | |
| 6:31958429delG− | CCTCGCTCAGTCCGGGGGTATC | 312 | CCAAC | 1312 |
| | ACCAACATGGTGGCTCCTAGTT | | ATGGT | |
| | CAGGGG | | GGCTCC | |
| | | | TAGTT | |
| 10:29471187del | CCCTGTTTCCTGCAGTGTTTCC | 313 | AGGGG | 1313 |
| C− | AGGGGGGATGGTGGTGCACTC | | GGATG | |
| | GGGGAG | | GTGGT | |
| | | | GCACTC | |
| 11:47175302del | CTGTGCATCCATGTTGGCGAAA | 314 | AAAAA | 1314 |
| A− | AAAGCCGTCTGGAGAGCAAAG | | AGCCG | |
| | AAG | | TCTGGA | |
| | | | GAGCA | |
| 4:7062246delA− | CCCTGCATTTTTTTTATGATGGC | 315 | GCTCA | 1315 |
| | TCAACAGCAAAGGTGTCTGGA | | ACAGC | |
| | GGAG | | AAAGG | |
| | | | TGTCTG | |
| 11:62882057del | CAAGGAAGATTTTGACAGTCTC | 316 | TTGCAA | 1316 |
| A+ | TTGCAATCGGCTAAAAAAAGA | | TCGGCT | |
| | GTGGGT | | AAAAA | |
| | | | AAGA | |
| 17:6787540delT+ | CTGAGCTGGTCATGTTGTCATG | 317 | GAGCT | 1317 |
| | GAGCTGACAAAAAAAAAAGTG | | GACAA | |
| | GAGGGG | | AAAAA | |
| | | | AAAGT | |
| | | | G | |
| 5:135334913del | CCCAGTAGGAGTGAAGGGGAT | 318 | TTTTTT | 1318 |
| T− | TTTTTTTTCTTTTAAACTGAAGG | | CTTTTA | |
| | TGGGGT | | AACTG | |
| | | | AAGG | |
| 1:52055208delT− | CTCAGCACAACACCTCTACTTC | 319 | CCAGA | 1319 |
| | CCAGATTTTTTTTTTCAAACTCT | | TTTTTT | |
| | GAAG | | TTTTCA | |
| | | | AACT | |
| 11:62882057del | CCAAGGAAGATTTTGACAGTCT | 320 | TCTCTT | 1320 |
| A+ | CTTGCAATCGGCTAAAAAAAG | | GCAAT | |
| | AGT | | CGGCT | |
| | | | AAAAA | |
| 1:40292750delT+ | CCTGGCAGCATGTTCCAGCTCT | 321 | TTGATG | 1321 |
| | TGATGTTTTTAAACTTTTTTTAG | | TTTTTA | |
| | AAG | | AACTTT | |
| | | | TTT | |
| 1:154584696del | CAAAGTTTTCAGTATCACCAAT | 322 | TTATGG | 1322 |
| A− | TATGGCTTAAAAAGAAAAAAA | | CTTAAA | |
| | AGGAG | | AAGAA | |
| | | | AAAA | |
| 5:37064968delA+ | CCAGGTTCCACTTACTCTTCAT | 323 | CATAA | 1323 |
| | AAAACTGATTTTTTTTGCCAGA | | AACTG | |
| | AT | | ATTTTT | |
| | | | TTTGC | |
| 1:10637121delT− | CAATGTAGCAAAATCAAACTTA | 324 | TAAAA | 1324 |
| | AAAAAAAAAAGAAGAAAAGA | | AAAAA | |
| | AGAAG | | AAGAA | |
| | | | GAAAA | |
| | | | G | |
| 2:186656358del | CAAGGGGGGACTGATGCAGTG | 325 | TGAGG | 1325 |
| G+ | TGAGGAATTGATAGCGTATCTG | | AATTG | |
| | CGGGT | | ATAGC | |
| | | | GTATCT | |
| 16:88624733del | CTCGGGAAGCCAGGAGGAAGG | 326 | AGCGG | 1326 |
| C+ | AGCGGCCAGCCAGGACCCCCC | | CCAGC | |
| | CAGGAG | | CAGGA | |
| | | | CCCCCC | |
| 7:100345930del | CTCAGTAATCTGAAAAAAAAA | 327 | AAATC | 1327 |
| T− | ATCCCTTCACATTTTAACTCAG | | CCTTCA | |
| | GAG | | CATTTT | |
| | | | AACT | |
| 8:94519335delA− | CTCGGATCAATAAATTTGTTGC | 328 | CTTCAT | 1328 |
| | TTCATATTCCGACATAAAAAAA | | ATTCCG | |
| | GAAG | | ACATA | |
| | | | AAAA | |
| 5:177248268del | CCCGGGATGCCTGCCTCTAAAA | 329 | AAAAA | 1329 |
| A+ | AATGCAGGGTGAACGCGGTGG | | TGCAG | |
| | AGG | | GGTGA | |
| | | | ACGCG | |
| | | | G | |
| 5:51393873delA+ | CAATGATCTTAGAAGTACTGAA | 330 | AAAAA | 1330 |
| | AAAAAAGACGTTTTTAAAACGT | | AGACG | |
| | AGAGG | | TTTTTA | |
| | | | AAACG | |
| 6:31536669delA− | CTCGGCAGGTTGCTGTTTTTTT | 331 | GTGGTC | 1331 |
| | GGTGGTCTGTCTATCAAGAAGG | | TGTCTA | |
| | ATGAAG | | TCAAG | |
| | | | AAGG | |
| 4:25847636delT− | CTGAGTTCAGAAGTAGCATTCC | 332 | TCCCGT | 1332 |
| | CGTGTACAAAAAAGGTGGAAG | | GTACA | |
| | AAT | | AAAAA | |
| | | | GGTGG | |
| 5:146511047del | CCGAGTTGGGGTTTGTTTTGTT | 333 | GTTTTG | 1333 |
| T+ | TTGATTTTTTTTTTTAAAGCGGG | | ATTTTT | |
| | T | | TTTTTT | |
| | | | AAA | |
| 19:19110944del | CATGGAACGGTGGGGGGTGCG | 334 | TGATTC | 1334 |
| G+ | CTTGATTCTACTTCAGGAGGCA | | TACTTC | |
| | CATGGGG | | AGGAG | |
| | | | GCAC | |
| 19:35719862del | CCGGGCAGGGGTCTTTGGGGG | 335 | TAGGA | 1335 |
| C+ | GTAGGAGCCACATCTGCTAGGC | | GCCAC | |
| | GAGGAG | | ATCTGC | |
| | | | TAGGC | |
| 17:30178149del | CTGTGTATGAATATGACAGTAT | 336 | TTATGA | 1336 |
| A+ | TTATGATGAAATGCAGAAAAA | | TGAAA | |
| | AAGGAG | | TGCAG | |
| | | | AAAAA | |
| 15:42450759del | CAAAGAATATCTCAAAAAACA | 337 | ACCAA | 1337 |
| A− | CCAAAACAAATTTTTTTCCCCT | | AACAA | |
| | GGAG | | ATTTTT | |
| | | | TTCCC | |
| 17:4972443delG− | CAGAGGCTGGCAGAGGTGCAG | 338 | GGGGA | 1338 |
| | GGGGGGACTGCCATCTGGGGC | | CTGCCA | |
| | ACTAGAAT | | TCTGGG | |
| | | | GCAC | |
| 3:171101725del | CACAGTTCTATATAGAGCTTTT | 339 | TTTTTT | 1339 |
| A− | TTTTTCTTGTTGCTTAAGCTGGA | | TTCTTG | |
| | G | | TTGCTT | |
| | | | AAG | |
| 2:238170406del | CATGGTTGTAAATAAAGGTTTC | 340 | TCTCTT | 1340 |
| A− | TCTTTTTTTTCCTAGTCTTTTGA | | TTTTTT | |
| | GT | | CCTAGT | |
| | | | CTT | |
| 14:93241685del | CCCAGCTCCTAGGACTTATTAA | 341 | AAAAA | 1341 |
| A− | AAAAAAATGACATTAGATTTAT | | AATGA | |
| | GGAGG | | CATTAG | |
| | | | ATTTA | |
| 19:35719862del | CTGTGCCTTCCTCACCCCGTTTC | 342 | CGGGC | 1342 |
| C+ | CGGGCAGGGGTCTTTGGGGGG | | AGGGG | |
| | TAGGAG | | TCTTTG | |
| | | | GGGGG | |
| 2:239052414del | CATCGGAAGATGCGAGTTTGTG | 343 | TGCCTT | 1343 |
| A− | CCTTTTTTTTATTGCTCTGGTGG | | TTTTTT | |
| | AT | | ATTGCT | |
| | | | CTG | |
| 12:107544001del | CAATGCCAAGCACCACGGCAA | 344 | GGCAC | 1344 |
| C+ | TGGCACCCCCTGCACCACAAGC | | CCCCTG | |
| | AGGGGG | | CACCA | |
| | | | CAAGC | |
| 3:14488912delT+ | CACAGTTGCTAGGGATTGGGA | 345 | ATTAAT | 1345 |
| | GATTAATTGGGTAAAAAAAAA | | TGGGT | |
| | AATGAAG | | AAAAA | |
| | | | AAAAA | |
| 10:29471187del | CCCTGTTTCCTGCAGTGTTTCC | 346 | CCAGG | 1346 |
| C− | AGGGGGGATGGTGGTGCACTC | | GGGGA | |
| | GGGG | | TGGTG | |
| | | | GTGCA | |
| | | | C | |
| 1:16135704delG− | CCTCGTACTTCCACACTCGGCT | 347 | CTGCTG | 1347 |
| | CTGCTGCGGCGGGGGATGCTCC | | CGGCG | |
| | AGGAG | | GGGGA | |
| | | | TGCTC | |
| 10:29471187del | CCCTGTTTCCTGCAGTGTTTCC | 348 | GGGGG | 1348 |
| C− | AGGGGGGATGGTGGTGCACTC | | GATGG | |
| | GGGGAGG | | TGGTGC | |
| | | | ACTCG | |
| 19:35719862del | CTTTGGGGGGTAGGAGCCACAT | 349 | CTGCTA | 1349 |
| C+ | CTGCTAGGCGAGGAGGAGGAA | | GGCGA | |
| | GGGGGG | | GGAGG | |
| | | | AGGAA | |
| 19:35719862del | CCGGGCAGGGGTCTTTGGGGG | 350 | AGGAG | 1350 |
| C+ | GTAGGAGCCACATCTGCTAGGC | | CCACAT | |
| | GAGGAGG | | CTGCTA | |
| | | | GGCG | |
| 8:107284561del | CTTTGTAATAGGTCTAGACAAA | 351 | AAAAA | 1351 |
| A− | AAAAAAGAATTTTCATTTTGAA | | AAGAA | |
| | GGAT | | TTTTCA | |
| | | | TTTTG | |
| 8:66430512delT+ | CGCCGTATATCCAACATTAAAA | 352 | AAAAG | 1352 |
| | AGAAAAAAAAGGCTGTTTAAG | | AAAAA | |
| | AAT | | AAAGG | |
| | | | CTGTTT | |
| 17:6787540delT+ | CTGAGCTGGTCATGTTGTCATG | 353 | TGGAG | 1353 |
| | GAGCTGACAAAAAAAAAAGTG | | CTGAC | |
| | GAGG | | AAAAA | |
| | | | AAAAA | |
| | | | G | |
| 8:144095973del | CAAGGAAGAGAGGAGGCCACG | 354 | CGGTG | 1354 |
| C+ | GTGAGACCACGGATAGCTGGG | | AGACC | |
| | GGGT | | ACGGA | |
| | | | TAGCTG | |
| 1:204954905del | CACAGCTGCTCATCCAGAAGAT | 355 | ACCGG | 1355 |
| C+ | GACCGGGGATGGAAGTCCAGG | | GGATG | |
| | CGGGGGT | | GAAGT | |
| | | | C | |
| 3:50100281delT+ | CCACGCTTAAAGTAACCATGCA | 356 | ACCGA | 1356 |
| | ACCGACTATAGTCAAAAAAAA | | CTATAG | |
| | AAGGAG | | TCAAA | |
| | | | AAAAA | |
| 5:51393873delA+ | CTACGTTTTAAAAACGTCTTTT | 357 | TTTTCA | 1357 |
| | TTTTCAGTACTTCTAAGATCAT | | GTACTT | |
| | TGAAT | | CTAAG | |
| | | | ATCA | |
| 11:112033459del | CACAGTTCAGAGATGGGAAAA | 358 | AAGTG | 1358 |
| A+ | AAAGTGGGTGAGAAGCTAAGT | | GGTGA | |
| | GAAGGAG | | GAAGC | |
| | | | TAAGT | |
| | | | G | |
| 14:93241685del | CCCAGCTCCTAGGACTTATTAA | 359 | AAAAA | 1359 |
| A− | AAAAAAATGACATTAGATTTAT | | AAATG | |
| | GGAG | | ACATTA | |
| | | | GATTT | |
| 17:70179777del | CTATGCCACACTTTTTTTTTCCC | 360 | CCACCT | 1360 |
| A+ | ACCTTAACATTATTAGACACAG | | TAACAT | |
| | AGT | | TATTAG | |
| | | | ACA | |
| 1:27550358delT− | CAGCGGCCACCATGGCCATGCC | 361 | AGAGG | 1361 |
| | AGAGGTAAAAAACGACGGCGG | | TAAAA | |
| | CGGAAG | | AACGA | |
| | | | CGGCG | |
| | | | G | |
| 4:7062246delA− | CCCTGCATTTTTTTTATGATGGC | 362 | TGGCTC | 1362 |
| | TCAACAGCAAAGGTGTCTGGA | | AACAG | |
| | GG | | CAAAG | |
| | | | GTGTC | |
| 10:26173831del | CAGAGTGTATCAGACTCCAAA | 363 | AAATG | 1363 |
| A+ | AAAATGAATAATGTGTATGAG | | AATAA | |
| | GAAGAGG | | TGTGTA | |
| | | | TGAGG | |
| 12:55028503del | CACAGTTGCCCATGGGAAAAA | 364 | AATGG | 1364 |
| T+ | CCAATGGATTTTTTTTAAGCAA | | ATTTTT | |
| | GATGAAT | | TTTAAG | |
| | | | CAAG | |
| 3:100355352del | CAAGGCCGAATTAAAAAAAAA | 365 | ATCTTC | 1365 |
| T+ | TCTTCATTAAAGAATTTAATAG | | ATTAA | |
| | GGAG | | AGAAT | |
| | | | TTAAT | |
| 8:13568072delT+ | CACAGATTTGAGGACTAAGAA | 366 | TTTCAC | 1366 |
| | CTTTCACTTTTTTTTCCTATTAT | | TTTTTT | |
| | AGAAG | | TTCCTA | |
| | | | TTA | |
| 15:78104908del | CCCTGGTCACCCAGGACAGAG | 367 | AGCGG | 1367 |
| T+ | CGGAAAAAAAAAAAGAGGTCA | | AAAAA | |
| | GGGT | | AAAAA | |
| | | | AGAGG | |
| | | | T | |
| 3:100355352del | CAAGGCCGAATTAAAAAAAAA | 368 | TCTTCA | 1368 |
| T+ | TCTTCATTAAAGAATTTAATAG | | TTAAA | |
| | GGAGT | | GAATTT | |
| | | | AATA | |
| 19:35719862del | CCGGGCAGGGGTCTTTGGGGG | 369 | GGTAG | 1369 |
| C+ | GTAGGAGCCACATCTGCTAGGC | | GAGCC | |
| | GAGG | | ACATCT | |
| | | | GCTAG | |
| 17:50356606del | CGTCGTAGGTGTTCTCCCAGTA | 370 | GCCTTG | 1370 |
| C+ | GGCCTTGAGGGCTGGCGTGCCG | | AGGGC | |
| | GGGGGT | | TGGCGT | |
| | | | GCCG | |
| 3:50100281delT+ | CCACGCTTAAAGTAACCATGCA | 371 | CCGACT | 1371 |
| | ACCGACTATAGTCAAAAAAAA | | ATAGTC | |
| | AAGGAGT | | AAAAA | |
| | | | AAAA | |
| 20:59028741del | CATTGTCAATTTATGAACAAGA | 372 | GACAG | 1372 |
| T− | CAGGATTTTTTTTTTCCCATGG | | GATTTT | |
| | AAT | | TTTTTT | |
| | | | CCCA | |
| 12:107544001del | CACGGCAATGGCACCCCCTGCA | 373 | CACAA | 1373 |
| C+ | CCACAAGCAGGGGGCACTGTA | | GCAGG | |
| | CTGGGAG | | GGGCA | |
| | | | CTGTAC | |
| 12:107544001del | CAATGCCAAGCACCACGGCAA | 374 | TGGCA | 1374 |
| C+ | TGGCACCCCCTGCACCACAAGC | | CCCCCT | |
| | AGGGG | | GCACC | |
| | | | ACAAG | |
| 6:31536669delA− | CTCGGCAGGTTGCTGTTTTTTT | 375 | TTTGGT | 1375 |
| | GGTGGTCTGTCTATCAAGAAGG | | GGTCTG | |
| | AT | | TCTATC | |
| | | | AAG | |
| 22:31089936del | CAGAGCCACCCCCAGCCCACCC | 376 | AAGAC | 1376 |
| C+ | AAGACCACCAGCCCTGAGCCTC | | CACCA | |
| | AGGAG | | GCCCTG | |
| | | | AGCCT | |
| 19:35719862del | CTTTGGGGGGTAGGAGCCACAT | 377 | TGCTAG | 1377 |
| C+ | CTGCTAGGCGAGGAGGAGGAA | | GCGAG | |
| | GGGGGGT | | GAGGA | |
| | | | GGAAG | |
| 19:35719862del | CTTTGGGGGGTAGGAGCCACAT | 378 | TCTGCT | 1378 |
| C+ | CTGCTAGGCGAGGAGGAGGAA | | AGGCG | |
| | GGGGG | | AGGAG | |
| | | | GAGGA | |
| 15:64169061del | CCTTGCAGCTGGATTTGCGACT | 379 | TTTTTT | 1379 |
| A− | TTTTTTTGTCTTAAAATTTTTAC | | GTCTTA | |
| | TGGAT | | AAATTT | |
| | | | TTA | |
| 17:30178149del | CTGTGTATGAATATGACAGTAT | 380 | TATGAT | 1380 |
| A+ | TTATGATGAAATGCAGAAAAA | | GAAAT | |
| | AAGGAGG | | GCAGA | |
| | | | AAAAA | |
| 5:128258372del | CAAGGAATATATGTTGTTGTTG | 381 | TGTTTT | 1381 |
| A− | TTGTTTTAAACCCATTTTTTTTT | | AAACC | |
| | AGAAT | | CATTTT | |
| | | | TTTT | |
| 17:6787540delT+ | CTGAGCTGGTCATGTTGTCATG | 382 | ATGGA | 1382 |
| | GAGCTGACAAAAAAAAAAGTG | | GCTGA | |
| | GAG | | CAAAA | |
| | | | AAAAA | |
| | | | A | |
| 5:1074583delG− | CTACGCCCTGCTGCGCGTGGAG | 383 | ACGGT | 1383 |
| | CACGGTCCCCCCACACCAAGA | | CCCCCC | |
| | ACTGGAG | | ACACC | |
| | | | AAGAA | |
| 19:47416569del | CTCTGCACCCCCCCACCAACCC | 384 | CCCAG | 1384 |
| C− | CAGGGATGGGGCGTCAGGGAA | | GGATG | |
| | GGAG | | GGGCG | |
| | | | TCAGG | |
| | | | G | |
| 16:10773346del | CCATGATATGGATTGTGTTTTT | 385 | TTTTTT | 1385 |
| A− | TTTAGCACCTTATTTTCCTTGAA | | AGCAC | |
| | G | | CTTATT | |
| | | | TTCC | |
| 1:25563141delT+ | CATGGCCAATCCCCCTTCCCCG | 386 | TCAGG | 1386 |
| | TCAGGACTCACAGCTCTTCAAG | | ACTCAC | |
| | GGGGG | | AGCTCT | |
| | | | TCAA | |
| 9:134154476del | CCAGGATGTATTTGCCGTTCGG | 387 | GAGAA | 1387 |
| C+ | GGAGAACTTCACAAAAGACAC | | CTTCAC | |
| | GGGGGGT | | AAAAG | |
| | | | ACACG | |
| 8:10726136delG− | CGGGGGACTGGCCAAGGGCCA | 388 | GGGAG | 1388 |
| | GGGAGCCCAGGGGTGGCTACA | | CCCAG | |
| | GTGGAG | | GGGTG | |
| | | | GCTAC | |
| | | | A | |
| 19:45052871del | CCTGGCAACCCCCTGGGAGGTG | 389 | CTTACA | 1389 |
| C+ | GCTTACATGGTGGTGAGCAGA | | TGGTG | |
| | GGGGGGT | | GTGAG | |
| | | | CAGAG | |
| 19:45052871del | CCTGGGAGGTGGCTTACATGGT | 390 | GGTGA | 1390 |
| C+ | GGTGAGCAGAGGGGGGTGTAG | | GCAGA | |
| | TCGGGG | | GGGGG | |
| | | | GTGTA | |
| | | | G | |
| 11:55886134del | CTGAGATTTTCACAAGCTTTTT | 391 | CCCATA | 1391 |
| A+ | CCCATAAAGACTGCATTTTTTT | | AAGAC | |
| | AGGAG | | TGCATT | |
| | | | TTTT | |
| 8:94880765delA+ | CTTAGTCACACTAAATTAAAAA | 392 | AAAAA | 1392 |
| | AAAAATTCCTTAGGGATATCTT | | TTCCTT | |
| | AGAGT | | AGGGA | |
| | | | TATCT | |
| 3:48158363delT− | CCAGGGACTACCTCGGCTTTTA | 393 | TTAATT | 1393 |
| | ATTTAAAAAAAAAAAGAAGTG | | TAAAA | |
| | GGT | | AAAAA | |
| | | | AAGAA | |
| 22:31089936del | CAGAGCCACCCCCAGCCCACCC | 394 | AGACC | 1394 |
| C+ | AAGACCACCAGCCCTGAGCCTC | | ACCAG | |
| | AGGAGT | | CCCTGA | |
| | | | GCCTC | |
| 2:120294679del | CATGGGCTTTTTTTTGAATAAA | 395 | AAGCA | 1395 |
| T+ | AAAGCAGACAAATAGACTTTCT | | GACAA | |
| | CGGGAT | | ATAGA | |
| | | | CTTTCT | |
| 19:35719862del | CTTTGGGGGGTAGGAGCCACAT | 396 | ATCTGC | 1396 |
| C+ | CTGCTAGGCGAGGAGGAGGAA | | TAGGC | |
| | GGGG | | GAGGA | |
| | | | GGAGG | |
| 3:51394249delT+ | CTAAGTTCTTAGATTTTGGGGG | 397 | GGGAT | 1397 |
| | ATTTTTTTTTTAAACGATGAGA | | TTTTTT | |
| | AG | | TTTAAA | |
| | | | CGAT | |
| 6:30189477delT− | CTGGGTTGGGAAACCCATTGCT | 398 | CGAGT | 1398 |
| | CGAGTGGTTAAAAAAAGACCG | | GGTTA | |
| | GAGAAT | | AAAAA | |
| | | | AGACC | |
| | | | G | |
| 8:144095973del | CCAAGGAAGAGAGGAGGCCAC | 399 | CGGTG | 1399 |
| C+ | GGTGAGACCACGGATAGCTGG | | AGACC | |
| | GGGGT | | ACGGA | |
| | | | TAGCTG | |
| 1:46609235delC | CACAGATGCCGGGGTGTGTGTG | 400 | TGTGTG | 1400 |
| A− | TGTGTGTGTATTTTCACTGTGG | | TGTGTA | |
| | GGT | | TTTTCA | |
| | | | CTG | |
| 9:66920942delA+ | CCAGGAAGACTGTGAGGGTTTT | 401 | TTCTTT | 1401 |
| | TCTTTTTTTTTTTAAGGGCCAAG | | TTTTTT | |
| | GGT | | TTAAG | |
| | | | GGCC | |
| 1:204954905del | CAGTGCAACCCCCGCCTGGACT | 402 | ACTTCC | 1402 |
| C+ | TCCATCCCCGGTCATCTTCTGG | | ATCCCC | |
| | AT | | GGTCAT | |
| | | | CTT | |
| 5:1074583delG− | CCCTGCTGCGCGTGGAGCACGG | 403 | CGGTCC | 1403 |
| | TCCCCCCACACCAAGAACTGGA | | CCCCAC | |
| | GG | | ACCAA | |
| | | | GAAC | |
| 9:93660330delA+ | CACAGTTTTGGGAGTCTTTTTTT | 404 | GGACA | 1404 |
| | GGACACTTTCTCCAGGAGGGAT | | CTTTCT | |
| | TGAGG | | CCAGG | |
| | | | AGGGA | |
| 15:42450759del | CAGGGGAAAAAAATTTGTTTTG | 405 | TGGTGT | 1405 |
| A− | GTGTTTTTTGAGATATTCTTTGG | | TTTTTG | |
| | AT | | AGATA | |
| | | | TTCT | |
| 2:238170406del | CCATGGTTGTAAATAAAGGTTT | 406 | TCTCTT | 1406 |
| A− | CTCTTTTTTTTCCTAGTCTTTTG | | TTTTTT | |
| | AGT | | CCTAGT | |
| | | | CTT | |
| 15:75207463del | CACAGGGGGGCAGTGCTGGGG | 407 | GGTAC | 1407 |
| C+ | GGTACAGGGAACTGGACTGTCT | | AGGGA | |
| | CGGAT | | ACTGG | |
| | | | ACTGTC | |
| 14:20992723del | CTAGGATGGGGATTCTTGGGAC | 408 | TTGCAT | 1408 |
| T+ | ATTGCATATGCATTTTTTTTTAA | | ATGCAT | |
| | AGAGG | | TTTTTT | |
| | | | TTA | |
| 6:28534234delA+ | CCATGCAGGAAGAGAGTGTGG | 409 | AGAAG | 1409 |
| | TGAGAAGATGGGTATTCCTTTT | | ATGGG | |
| | TTTGGAG | | TATTCC | |
| | | | TTTTT | |
| 1:52055208delT− | CCCAGATTTTTTTTTTCAAACTC | 410 | CTCTGA | 1410 |
| | TGAAGGAAGTGATGTTAGACG | | AGGAA | |
| | GAT | | GTGAT | |
| | | | GTTAG | |
| 3:100355352del | CCTAGGAAGGACAAGGCCGAA | 411 | ATTAA | 1411 |
| T+ | TTAAAAAAAAATCTTCATTAAA | | AAAAA | |
| | GAAT | | AATCTT | |
| | | | CATTA | |
| 19:47416569del | CAATGGGTCTCCTTCCCTGACG | 412 | CCCCAT | 1412 |
| C− | CCCCATCCCTGGGGTTGGTGGG | | CCCTGG | |
| | GGGGT | | GGTTG | |
| | | | GTGG | |
| 1:25563141delT+ | CATGGCCAATCCCCCTTCCCCG | 413 | CGTCA | 1413 |
| | TCAGGACTCACAGCTCTTCAAG | | GGACT | |
| | GGG | | CACAG | |
| | | | CTCTTC | |
| 1:46609235delC | CACAGATGCCGGGGTGTGTGTG | 414 | GTGTGT | 1414 |
| A− | TGTGTGTGTATTTTCACTGTGG | | GTGTGT | |
| | GG | | ATTTTC | |
| | | | ACT | |
| 4:139266508del | CTTAGTTTAAAAAAAAAAGACT | 415 | CTTATT | 1415 |
| T− | TATTTTCTAGAAAACGTTAATG | | TTCTAG | |
| | GGT | | AAAAC | |
| | | | GTTA | |
| 1:204954905del | CCGGGGATGGAAGTCCAGGCG | 416 | GGGGG | 1416 |
| C+ | GGGGTTGCACTGGAGCGTCAA | | TTGCAC | |
| | AGGAG | | TGGAG | |
| | | | CGTCA | |
| 10:26173831del | CAGAGTGTATCAGACTCCAAA | 417 | AAAAA | 1417 |
| A+ | AAAATGAATAATGTGTATGAG | | ATGAA | |
| | GAAG | | TAATGT | |
| | | | GTATG | |
| 12:45920608del | CAGGGATTAGGGATTTGGGTTT | 418 | TTTTTT | 1418 |
| A− | TTTTTTTTTCTCTTTTTAATACT | | TTCTCT | |
| | AGAAT | | TTTTAA | |
| | | | TAC | |
| 11:112033459del | CACAGTTCAGAGATGGGAAAA | 419 | AAAAA | 1419 |
| A+ | AAAGTGGGTGAGAAGCTAAGT | | GTGGG | |
| | GAAG | | TGAGA | |
| | | | AGCTA | |
| | | | A | |
| 9:35043653delC+ | CTGGGTTCTGACAGAGAAGCTG | 420 | GGGAT | 1420 |
| | GGGATTGGCAGGGGGTGGCAT | | TGGCA | |
| | CGGAGG | | GGGGG | |
| | | | TGGCAT | |
| 11:70468159del | CACAGAAAAGAAAAAAAAAAG | 421 | AAAAA | 1421 |
| T− | GAAAAAAAATAAAGTGTGTGC | | AAATA | |
| | CTTGGGT | | AAGTG | |
| | | | TGTGCC | |
| 1:25563141delT+ | CATGGCCAATCCCCCTTCCCCG | 422 | GTCAG | 1422 |
| | TCAGGACTCACAGCTCTTCAAG | | GACTC | |
| | GGGG | | ACAGC | |
| | | | TCTTCA | |
| 19:47416569del | CATGGACCAGGTGGCCTTCTCT | 423 | CTGCAC | 1423 |
| C− | CTGCACCCCCCCACCAACCCCA | | CCCCCC | |
| | GGGAT | | ACCAA | |
| | | | CCCC | |
| 11:44265074del | CAGAGCCAGGGGGGGGCATG | 424 | GAGGG | 1424 |
| G− | AGGGGACATGCAGGCAGGCAC | | GACAT | |
| | CGGGT | | GCAGG | |
| | | | CAGGC | |
| | | | A | |
| 1:26780313delC+ | CATAGTGCTATACAACTTCTCC | 425 | GGCGG | 1425 |
| | AGGCGGCTGAAGGGGGTGTGG | | CTGAA | |
| | CCAGAAT | | GGGGG | |
| | | | TGTGGC | |
| 5:177248268del | CCGGGATGCCTGCCTCTAAAAA | 426 | AAATG | 1426 |
| A+ | ATGCAGGGTGAACGCGGTGGA | | CAGGG | |
| | GGAG | | TGAAC | |
| | | | GCGGT | |
| | | | G | |
| 1:25563141delT+ | CATGGCCAATCCCCCTTCCCCG | 427 | CAGGA | 1427 |
| | TCAGGACTCACAGCTCTTCAAG | | CTCACA | |
| | GGGGGG | | GCTCTT | |
| | | | CAAG | |
| 4:17624016delT+ | CCAAGTAAGTATTTTTTTTTGTC | 428 | GTCTTT | 1428 |
| | TTTAGCAAAGTTTAGACTGTGA | | AGCAA | |
| | AT | | AGTTTA | |
| | | | GACT | |
| 14:73739070del | CCTTGCGCAGTTCTGGGTTCAT | 429 | ATCTGG | 1429 |
| G− | ATCTGGGTTGGGGGGAAGGGG | | GTTGG | |
| | TAGGGT | | GGGGA | |
| | | | AGGGG | |
| 6:80010913delA+ | CCCTGCGGAATTTAAACCTCCA | 430 | CAAAA | 1430 |
| | AAAAAGCAGCTGCTTTCAGAG | | AAGCA | |
| | GAGG | | GCTGCT | |
| | | | TTCAG | |
| 10:29471187del | CAGTGTTTCCAGGGGGGATGGT | 431 | TGGTGC | 1431 |
| C− | GGTGCACTCGGGGAGGCGGGA | | ACTCG | |
| | AGAGG | | GGGAG | |
| | | | GCGGG | |
| 2:239081127del | CAACGTCAACATGGCTTTCACC | 432 | ACCGG | 1432 |
| G− | GGCGGCCTGGACCCCCCATGG | | CGGCCT | |
| | GAG | | GGACC | |
| | | | CCCCA | |
| 22:37373064del | CCCAGCAGAAGCTGTGACCCCC | 433 | CCCCCT | 1433 |
| G− | CCTTCCTCCCTGGTGAGGTCGG | | TCCTCC | |
| | AG | | CTGGTG | |
| | | | AGG | |
| 4:92304602delA+ | CACCGTGACCTCAAACTCTTTG | 434 | GACTGT | 1434 |
| | GACTGTTTGAAAAAAAAAAAT | | TTGAA | |
| | TGGAAG | | AAAAA | |
| | | | AAAAT | |
| 16:28836029del | CTCTGGGGCACCGCGCCTTGGG | 435 | GGGGG | 1435 |
| G+ | GGGGCCCCCATGACTCTGGGGT | | CCCCCA | |
| | GGGT | | TGACTC | |
| | | | TGGG | |
| 8:22082312delC+ | CTGGGGGGCCCCAATAAATTAC | 436 | ATTCTT | 1436 |
| | ATTCTTGAGAGAGCATAGTGTG | | GAGAG | |
| | TGGGG | | AGCAT | |
| | | | AGTGT | |
| 1:100872563del | CTTTGTGCATTTAGTTCCGCAT | 437 | TGATG | 1437 |
| A− | ATGATGGTTTTTTTTTACATTAA | | GTTTTT | |
| | AGAGT | | TTTTAC | |
| | | | ATTA | |
| 5:140669517del | CTGCGAGCTGTGGTGGTGGATG | 438 | ACTACC | 1438 |
| A+ | ACTACCGTCGGCGCAAAAAAA | | GTCGG | |
| | GGGAGG | | CGCAA | |
| | | | AAAAA | |
| 12:122603073del | CTCTGTACGTGTCTACAGCAAA | 439 | AAAAC | 1439 |
| A+ | ACACGTTTTCGAAAAAAACTGA | | ACGTTT | |
| | AG | | TCGAA | |
| | | | AAAAA | |
| 5:37064968delA+ | CCCAGGTTCCACTTACTCTTCA | 440 | CATAA | 1440 |
| | TAAAACTGATTTTTTTTGCCAG | | AACTG | |
| | AAT | | ATTTTT | |
| | | | TTTGC | |
| 12:7089414delG− | CCTCGGTCCTACCCCCTGACCT | 44 | CGCTGC | 1441 |
| | GCGCTGCAACTACAGCATCCGG | | AACTA | |
| | GTGGAG | | CAGCA | |
| | | | TCCGG | |
| 19:4816453delG− | CTGTGGGGAGGGCGGTGGGGG | 442 | GGTGC | 1442 |
| | GTGCCAGCCTGCCATGCGTGCA | | CAGCCT | |
| | GGGG | | GCCAT | |
| | | | GCGTG | |
| 4:38689855delC+ | CGGGGGGACACTGAGTTCATTA | 443 | TTAAG | 1443 |
| | AGGGGGGTGACATTTCTTCAGG | | GGGGG | |
| | AT | | TGACAT | |
| | | | TTCTT | |
| 2:239081127del | CATGGCTTTCACCGGCGGCCTG | 444 | CTGGA | 1444 |
| G− | GACCCCCCATGGGAGACGCTG | | CCCCCC | |
| | AGT | | ATGGG | |
| | | | AGACG | |
| 2:205301574del | CATGGAAAATAAAGCCAGGAA | 445 | AGTCA | 1445 |
| A+ | AGTCAAAAAACGAAAGAGAAG | | AAAAA | |
| | GAGAAG | | CGAAA | |
| | | | GAGAA | |
| | | | G | |
| 19:4816453delG− | CTGTGGGGAGGGCGGTGGGGG | 446 | GTGCC | 1446 |
| | GTGCCAGCCTGCCATGCGTGCA | | AGCCT | |
| | GGGGT | | GCCAT | |
| | | | GCGTG | |
| | | | C | |
| 9:32633586delT− | CTTGGCCTTTTTTTGATGTGCTT | 447 | CTTTAG | 1447 |
| | TAGCAAAGGTTGGACTGAATG | | CAAAG | |
| | GGG | | GTTGG | |
| | | | ACTGA | |
| 1:27550358delT− | CCATGGCCATGCCAGAGGTAA | 448 | AAAAA | 1448 |
| | AAAACGACGGCGGCGGAAGCA | | ACGAC | |
| | GAAG | | GGCGG | |
| | | | CGGAA | |
| | | | G | |
| 20:32434639del | CATAGAGAGGCGGCCACCACT | 449 | TGCCAT | 1449 |
| G+ | GCCATCGGAGGGGGGGTGGCC | | CGGAG | |
| | CGGGT | | GGGGG | |
| | | | GTGGC | |
| 8:22082312delC+ | CTGGGGGGCCCCAATAAATTAC | 450 | TTCTTG | 1450 |
| | ATTCTTGAGAGAGCATAGTGTG | | AGAGA | |
| | TGGGGG | | GCATA | |
| | | | GTGTG | |
| 16:88624733del | CCTGGGGGGGTCCTGGCTGGCC | 451 | GCTCCT | 1451 |
| C+ | GCTCCTTCCTCCTGGCTTCCCG | | TCCTCC | |
| | AGGGT | | TGGCTT | |
| | | | CCC | |
| 5:140669517del | CTGCGAGCTGTGGTGGTGGATG | 452 | GACTA | 1452 |
| A+ | ACTACCGTCGGCGCAAAAAAA | | CCGTCG | |
| | GGGAG | | GCGCA | |
| | | | AAAAA | |
| 6:30653271delT− | CTAAGAAAATGCCCAAAAAAT | 453 | TAGGC | 1453 |
| | AGGCAAAACACGAGAAGAGCT | | AAAAC | |
| | AGGGT | | ACGAG | |
| | | | AAGAG | |
| | | | C | |
| 14:20992723del | CGGAGATCCTCTTTAAAAAAAA | 454 | AAAAT | 1454 |
| T+ | ATGCATATGCAATGTCCCAAGA | | GCATAT | |
| | AT | | GCAAT | |
| | | | GTCCC | |
| X:129805329del | CTCAGCCGAGGCTTAATGGAA | 455 | ACTGGT | 1455 |
| T− | GAACTGGTTAGCATTTTTTTTTT | | TAGCAT | |
| | TTGAGG | | TTTTTT | |
| | | | TTT | |
| 3:127016548del | CTCTGCCCGGGGGTGTCAGGCA | 456 | CGGAT | 1456 |
| C+ | CCGGATCTCACGGGAGTTCCTC | | CTCACG | |
| | CTGGAG | | GGAGT | |
| | | | TCCTC | |
| 10:29471187del | CCAGGGGGGATGGTGGTGCAC | 457 | ACTCG | 1457 |
| C− | TCGGGGAGGCGGGAAGAGGAA | | GGGAG | |
| | GAAG | | GCGGG | |
| | | | AAGAG | |
| | | | G | |
| 3:51380173delC+ | CTGGGGGGTATCACCCAGAGG | 458 | TGGTG | 1458 |
| | GTGGTGGAAGGCGTCAAAGTG | | GAAGG | |
| | TAGGGAG | | CGTCA | |
| | | | AAGTG | |
| | | | T | |
| 1:1354730delG− | CCCCGGGGGGCGGCTCCGCGT | 459 | TGGGG | 1459 |
| | GGGGTTCGGCGACCGTCAGGT | | TTCGGC | |
| | GGAAG | | GACCG | |
| | | | TCAGG | |
| 9:35043653delC+ | CTGGGTTCTGACAGAGAAGCTG | 460 | GGGGA | 1460 |
| | GGGATTGGCAGGGGGTGGCAT | | TTGGCA | |
| | CGGAG | | GGGGG | |
| | | | TGGCA | |
| 12:6602380delT− | CCTCGGGACCCTAAAATCCCTA | 461 | GAGCA | 1461 |
| | AGAGCAAGCGCCAAAAAAGGA | | AGCGC | |
| | GGTGAGT | | CAAAA | |
| | | | AAGGA | |
| | | | G | |
| 7:997675delG− | CCCTGATGCGGGAGCTGGATG | 462 | GGAGG | 1462 |
| | AGGAGGGCTCTGATCCCCCCTG | | GCTCTG | |
| | CCGGGG | | ATCCCC | |
| | | | CCTG | |
| 6:30189477delT− | CTGGGTTGGGAAACCCATTGCT | 463 | GCTCG | 1463 |
| | CGAGTGGTTAAAAAAAGACCG | | AGTGG | |
| | GAG | | TTAAA | |
| | | | AAAAG | |
| | | | A | |
| 20:32434639del | CGGAGGGGGGGTGGCCCGGGT | 464 | GAGGT | 1464 |
| G+ | GGAGGTGGCGGCGGGGCCACC | | GGCGG | |
| | GATGAGG | | CGGGG | |
| | | | CCACC | |
| | | | G | |
| 16:28836029del | CAGAGTCATGGGGGCCCCCCC | 465 | CCAAG | 1465 |
| G+ | AAGGCGCGGTGCCCCAGAGTG | | GCGCG | |
| | GGGT | | GTGCCC | |
| | | | CAGAG | |
| 22:37373064del | CTGTGACCCCCCCTTCCTCCCT | 466 | GGTGA | 1466 |
| G− | GGTGAGGTCGGAGCCAGAGGG | | GGTCG | |
| | CTGGGG | | GAGCC | |
| | | | AGAGG | |
| | | | G | |
| 19:45052871del | CCCTGGGAGGTGGCTTACATGG | 467 | GGTGA | 1467 |
| C+ | TGGTGAGCAGAGGGGGGTGTA | | GCAGA | |
| | GTCGGGG | | GGGGG | |
| | | | GTGTA | |
| | | | G | |
| 6:80010913delA+ | CCCTGCGGAATTTAAACCTCCA | 468 | CCAAA | 1468 |
| | AAAAAGCAGCTGCTTTCAGAG | | AAAGC | |
| | GAG | | AGCTG | |
| | | | CTTTCA | |
| 19:49347216del | CCAGGAAGAAGGCATGGGGGG | 469 | GGGCC | 1469 |
| G− | GCCACGATCATATAGCTCTCGG | | ACGAT | |
| | AGG | | CATATA | |
| | | | GCTCT | |
| 11:2130529delG− | CATGGGGGGGGGTTTAATTTGG | 470 | TTCTGA | 1470 |
| | TTTCTGAGCGCATAAAGCTAAG | | GCGCA | |
| | GAGGGG | | TAAAG | |
| | | | CTAAG | |
| 20:32453753del | CGTAGCTCCCAGAGCTGTAGGG | 471 | GGGGG | 1471 |
| G− | GGGGACTAAAAGGAGGGCAAG | | GGACT | |
| | AGG | | AAAAG | |
| | | | GAGGG | |
| | | | C | |
| 11:2130529delG− | CATGGGGGGGGGTTTAATTTGG | 472 | GTTTCT | 1472 |
| | TTTCTGAGCGCATAAAGCTAAG | | GAGCG | |
| | GAGG | | CATAA | |
| | | | AGCTA | |
| 11:44265074del | CGGTGCCTGCCTGCATGTCCCC | 473 | CCTCAT | 1473 |
| G− | TCATGCCCACCCCCTGGCTCTG | | GCCCA | |
| | GGG | | CCCCCT | |
| | | | GGCT | |
| 15:89200730del | CAAAGCATGCAGAGTGCTATTT | 474 | TTTCTT | 1474 |
| A+ | CTTTTTTTTTCTCTTGACCAGAA | | TTTTTT | |
| | G | | TCTCTT | |
| | | | GAC | |
| 17:36943098del | CCGTGCGAGACCCCGCTACCAC | 475 | ACGGC | 1475 |
| C+ | ACGGCCGCCTCGTTCATTTCGG | | CGCCTC | |
| | GGGGT | | GTTCAT | |
| | | | TTCG | |
| 8:66430512delT+ | CCTCGCCGTATATCCAACATTA | 476 | AAAAG | 1476 |
| | AAAAGAAAAAAAAGGCTGTTT | | AAAAA | |
| | AAGAAT | | AAAGG | |
| | | | CTGTTT | |
| 5:140852886del | CTATGTCATCAATAATCATAAA | 477 | AAACG | 1477 |
| T+ | ACGTATTTTTTTTTTGAGTCAG | | TATTTT | |
| | AGT | | TTTTTT | |
| | | | GAGT | |
| 3:51380173delC+ | CTGGGGGGTATCACCCAGAGG | 478 | GGTGG | 1478 |
| | GTGGTGGAAGGCGTCAAAGTG | | AAGGC | |
| | TAGGGAGT | | GTCAA | |
| | | | AGTGT | |
| | | | A | |
| 11:112033459del | CCATGACCATGGGCACAGTTCA | 479 | GAGAT | 1479 |
| A+ | GAGATGGGAAAAAAAGTGGGT | | GGGAA | |
| | GAGAAG | | AAAAA | |
| | | | GTGGG | |
| | | | T | |
| 9:137838509del | CAGGGGGCTCTGCTCTCCCTTG | 480 | CTGAC | 1480 |
| C+ | CCTGACAGGAGACGCACTCGG | | AGGAG | |
| | CCCGAGT | | ACGCA | |
| | | | CTCGGC | |
| 10:46809223del | CAGAGTGAGACTCCCTCTCAAA | 481 | AAAAA | 1481 |
| T− | AAAAAAAAAAGAGAGAGAGCG | | AAAAA | |
| | AGAAT | | AGAGA | |
| | | | GAGAG | |
| | | | C | |
| X:12977149delT+ | CAGAGTGCCATTTTTTTTTTGTT | 482 | TGTTCA | 1482 |
| | CAAATGATTTTAATTATTGGAA | | AATGA | |
| | T | | TTTTAA | |
| | | | TTAT | |
| 1:71404165delT− | CAGGGAAAAAAAAAATATATA | 483 | TATATA | 1483 |
| | TATATATAAATACCCCTACATT | | TAAAT | |
| | TGAAG | | ACCCCT | |
| | | | ACAT | |
| 1:156745312del | CCAAGTCTTTTTTTCGGGACCC | 484 | ACGAG | 1484 |
| A− | ACGAGACGTGAGTGGAGGCCA | | ACGTG | |
| | AAGGGG | | AGTGG | |
| | | | AGGCC | |
| | | | A | |
| 18:36625553del | CTCAGGCACGAGGATGGCGAT | 485 | GAGAC | 1485 |
| C+ | GAGACCACGGAGCCACCCCCA | | CACGG | |
| | GTGGGT | | AGCCA | |
| | | | CCCCCA | |
| 2:186656358del | CCAAGAACATGACTATTTCAAG | 486 | AGGGG | 1486 |
| G+ | GGGGGACTGATGCAGTGTGAG | | GGACT | |
| | GAAT | | GATGC | |
| | | | AGTGT | |
| | | | G | |
| 16:88624733del | CCTCGGGAAGCCAGGAGGAAG | 487 | AGCGG | 1487 |
| C+ | GAGCGGCCAGCCAGGACCCCC | | CCAGC | |
| | CCAGGAG | | CAGGA | |
| | | | CCCCCC | |
| 2:1649188delG− | CCTGGCCCGGGAGTCATTGGGG | 488 | GGATC | 1488 |
| | GGATCATGACAGAGAAGCAGG | | ATGAC | |
| | GGGGGT | | AGAGA | |
| | | | AGCAG | |
| | | | G | |
| 20:49636246del | CACCGCCACCAAGAAAGCAGT | 489 | CGATG | 1489 |
| A− | CCGATGAGATTTTTTTTGGAGG | | AGATTT | |
| | GGGGAG | | TTTTTG | |
| | | | GAGG | |
| 12:109581435del | CCCTGCCGAGCCTGGATATCGT | 490 | GTAGT | 1490 |
| C+ | AGTGTGGTCGGAGCTGCCCCCG | | GTGGTC | |
| | GGG | | GGAGC | |
| | | | TGCCC | |
| X:154409207del | CACAGCCTCTTCCTCTTTTTTTC | 49 | CCCCTC | 1491 |
| A− | CCCTCCTAGCCCTATTCAGGCA | | CTAGCC | |
| | GGAG | | CTATTC | |
| | | | AGG | |
| 22:37373064del | CTGTGACCCCCCCTTCCTCCCT | 492 | GTGAG | 1492 |
| G− | GGTGAGGTCGGAGCCAGAGGG | | GTCGG | |
| | CTGGGGG | | AGCCA | |
| | | | GAGGG | |
| | | | C | |
| 15:44711583del | CTCCGTGGCCTTAGCTGTGCTC | 493 | GCGCT | 1493 |
| CT+ | GCGCTATCTCTCTTTCTGGCCT | | ATCTCT | |
| | GGAGG | | CTTTCT | |
| | | | GGCC | |
| 12:7089414delG− | CCTCGGTCCTACCCCCTGACCT | 494 | CCTGCG | 1494 |
| | GCGCTGCAACTACAGCATCCGG | | CTGCA | |
| | GT | | ACTAC | |
| | | | AGCAT | |
| 11:66271672del | CAGAGTGAGACCTTATTGCTAA | 495 | AAAAA | 1495 |
| A+ | AAAAAAATAAAAATAAACCAA | | AAATA | |
| | GGGAT | | AAAAT | |
| | | | AAACC | |
| | | | A | |
| 1:154963387del | CAAAGGCACAAAGTTTAAACA | 496 | CATGG | 1496 |
| G− | TGGGGGGGCGGGTGTTGAGAG | | GGGGG | |
| | GGGT | | CGGGT | |
| | | | GTTGA | |
| | | | G | |
| 19:47416569del | CCAGGTGGCCTTCTCTCTGCAC | 497 | ACCCCC | 1497 |
| C− | CCCCCCACCAACCCCAGGGATG | | CCACC | |
| | GGG | | AACCC | |
| | | | CAGGG | |
| 2:200818782del | CAAGGAGGAGATGAAAAAAAC | 498 | CATCCC | 1498 |
| A+ | AACATCCCAGAGCCAGTTGTCA | | AGAGC | |
| | TCGGAAT | | CAGTTG | |
| | | | TCAT | |
| 3:100355352del | CTAGGAAGGACAAGGCCGAAT | 499 | ATTAA | 1499 |
| T+ | TAAAAAAAAATCTTCATTAAAG | | AAAAA | |
| | AAT | | AATCTT | |
| | | | CATTA | |
| 8:10726136delG− | CCAAGGGCCAGGGAGCCCAGG | 500 | GGTGG | 1500 |
| | GGTGGCTACAGTGGAGAGGGC | | CTACA | |
| | TTGGGG | | GTGGA | |
| | | | GAGGG | |
| | | | C | |
| 11:2130529delG− | CATGGGGGGGGGTTTAATTTGG | 501 | GGTTTC | 1501 |
| | TTTCTGAGCGCATAAAGCTAAG | | TGAGC | |
| | GAG | | GCATA | |
| | | | AAGCT | |
| 12:107544001del | CCCTGCTTGTGGTGCAGGGGGT | 502 | CCATTG | 1502 |
| C+ | GCCATTGCCGTGGTGCTTGGCA | | CCGTG | |
| | TTGAGG | | GTGCTT | |
| | | | GGCA | |
| 1:157697170del | CACAGCATCAAAAAAGGAGCC | 503 | CTGAG | 1503 |
| T− | TGAGATCTCAGATACGTGTACA | | ATCTCA | |
| | GAGT | | GATAC | |
| | | | GTGTA | |
| 16:28836029del | CTGGGGCACCGCGCCTTGGGG | 504 | GGGGG | 1504 |
| G+ | GGGCCCCCATGACTCTGGGGTG | | CCCCCA | |
| | GGT | | TGACTC | |
| | | | TGGG | |
| 16:88624733del | CCTGGGGGGGTCCTGGCTGGCC | 505 | CCGCTC | 1505 |
| C+ | GCTCCTTCCTCCTGGCTTCCCG | | CTTCCT | |
| | AGG | | CCTGGC | |
| | | | TTC | |
| 15:44711583del | CTCCGTGGCCTTAGCTGTGCTC | 506 | CGCGCT | 1506 |
| CT+ | GCGCTATCTCTCTTTCTGGCCT | | ATCTCT | |
| | GGAG | | CTTTCT | |
| | | | GGC | |
| 17:61482984del | CGTAGGGAGGGGGGAACGGAA | 507 | TAGTG | 1507 |
| C+ | ATAGTGATCCTCCCCCACCGAA | | ATCCTC | |
| | GAGGGG | | CCCCAC | |
| | | | CGAA | |
| 1:156673012del | CTCTGCATCTACAGCAGGAGAG | 508 | GGTGC | 1508 |
| G− | GGTGCCTGAGGTGTGGGGGGA | | CTGAG | |
| | TGGGGG | | GTGTG | |
| | | | GGGGG | |
| | | | A | |
| 16:88624733del | CCTCGGGAAGCCAGGAGGAAG | 509 | GCGGC | 1509 |
| C+ | GAGCGGCCAGCCAGGACCCCC | | CAGCC | |
| | CCAGGAGG | | AGGAC | |
| | | | CCCCCC | |
| 8:10726136delG− | CAAGGGCCAGGGAGCCCAGGG | 510 | GGTGG | 1510 |
| | GTGGCTACAGTGGAGAGGGCT | | CTACA | |
| | TGGGG | | GTGGA | |
| | | | GAGGG | |
| | | | C | |
| 2:1649188delG− | CCTGGCCCGGGAGTCATTGGGG | 511 | GGGAT | 1511 |
| | GGATCATGACAGAGAAGCAGG | | CATGA | |
| | GGGGG | | CAGAG | |
| | | | AAGCA | |
| | | | G | |
| 19:38730431del | CCAAGAAAAAAAATCAATCAG | 512 | AATAA | 1512 |
| T+ | AATAAACTCAAAAAAAAAGGT | | ACTCA | |
| | AGGGGG | | AAAAA | |
| | | | AAAGG | |
| | | | T | |
| X:91436820delT+ | CTTTGTGATAAGGGGTTATTTT | 513 | ATGCTA | 1513 |
| | ATGCTAATTCACAAGTTTTTTTT | | ATTCAC | |
| | GAAG | | AAGTTT | |
| | | | TTT | |
| 19:38730431del | CCAAGAAAAAAAATCAATCAG | 514 | TAAACT | 1514 |
| T+ | AATAAACTCAAAAAAAAAGGT | | CAAAA | |
| | AGGGGGAG | | AAAAA | |
| | | | GGTAG | |
| 8:66453963delT+ | CTTAGGGAAAGATATGGTGAA | 515 | AAAAA | 1515 |
| | AAAAAAGAAATGCTACTCGGT | | AGAAA | |
| | AGGAAG | | TGCTAC | |
| | | | TCGGT | |
| 7:44080815delG− | CCCAGGCTCTCTGTTAACCAGC | 516 | AGCTTA | 1516 |
| | TTATGTCCAGCAGAGCTGGGGG | | TGTCCA | |
| | GT | | GCAGA | |
| | | | GCTG | |
| 19:38730431del | CCAAGAAAAAAAATCAATCAG | 517 | GAATA | 1517 |
| T+ | AATAAACTCAAAAAAAAAGGT | | AACTC | |
| | AGGGG | | AAAAA | |
| | | | AAAAG | |
| | | | G | |
| 12:7089414delG− | CCCGGATGCTGTAGTTGCAGCG | 518 | CGCAG | 1518 |
| | CAGGTCAGGGGGTAGGACCGA | | GTCAG | |
| | GGGT | | GGGGT | |
| | | | AGGAC | |
| | | | C | |
| 20:57652293del | CGTAGCACGTGGCGCTGATGCC | 519 | GCCCG | 1519 |
| G− | CGAGTTACTGCTGGGGGGCAG | | AGTTAC | |
| | GGG | | TGCTGG | |
| | | | GGGG | |
| 1:156745312del | CCAAGTCTTTTTTTCGGGACCC | 520 | CGAGA | 1520 |
| A− | ACGAGACGTGAGTGGAGGCCA | | CGTGA | |
| | AAGGGGT | | GTGGA | |
| | | | GGCCA | |
| | | | A | |
| 20:49636246del | CACCGCCACCAAGAAAGCAGT | 521 | GATGA | 1521 |
| A− | CCGATGAGATTTTTTTTGGAGG | | GATTTT | |
| | GGGGAGG | | TTTTGG | |
| | | | AGGG | |
| 10:100827567del | CAGCGGCAGGGGCGGAGCCCC | 522 | CGGGG | 1522 |
| C+ | GGGGGCGGCACTATAATAATA | | GCGGC | |
| | AGGGG | | ACTATA | |
| | | | ATAAT | |
| 19:11453958del | CAGTGGGGCTGGAAGCAGAAA | 523 | AAAAT | 1523 |
| T− | CAAAATGAAAAAAAAGGGGGG | | GAAAA | |
| | TGGGAGG | | AAAAG | |
| | | | GGGGG | |
| | | | T | |
| X:130056036del | CCTGGAGGCTTGGACGACAGA | 524 | CCCCCA | 1524 |
| C+ | TCCCCCCAGGCTCCTCTGAGAC | | GGCTCC | |
| | TGTGGAG | | TCTGAG | |
| | | | ACT | |
| 11:44265074del | CCAGGGGGTGGGCATGAGGGG | 525 | ATGCA | 1525 |
| G− | ACATGCAGGCAGGCACCGGGT | | GGCAG | |
| | CGCAGGGG | | GCACC | |
| | | | GGGTC | |
| | | | G | |
| 20:49636246del | CACCGCCACCAAGAAAGCAGT | 526 | TCCGAT | 1526 |
| A− | CCGATGAGATTTTTTTTGGAGG | | GAGAT | |
| | GGGG | | TTTTTT | |
| | | | TGGA | |
| 19:19110944del | CTGGGCCCATGGAACGGTGGG | 527 | GGGGT | 1527 |
| G+ | GGGTGCGCTTGATTCTACTTCA | | GCGCTT | |
| | GGAG | | GATTCT | |
| | | | ACTT | |
| 2:66568967delT | CAAAGTCAACAGATAGTGCCA | 528 | CAAAA | 1528 |
| T+ | AAAGACCCTTAAAAAAAAACA | | GACCCT | |
| | GGAT | | TAAAA | |
| | | | AAAAA | |
| 2:66568967delT+ | CAAAGTCAACAGATAGTGCCA | 529 | CAAAA | 1529 |
| | AAAGACCCTTAAAAAAAAACA | | GACCCT | |
| | GGAT | | TAAAA | |
| | | | AAAAA | |
| 20:59945733del | CTCTGACATGGTTTTTTTTTCTT | 530 | TTTTGA | 1530 |
| T+ | TTTTGAGGGGCATTTTAAACTT | | GGGGC | |
| | AGAGG | | ATTTTA | |
| | | | AACT | |
| 6:31958429delG− | CCCTGAACTAGGAGCCACCATG | 531 | TTGGTG | 1531 |
| | TTGGTGATACCCCCGGACTGAG | | ATACCC | |
| | CGAGG | | CCGGA | |
| | | | CTGA | |
| 8:10726136delG− | CCCAGGGGTGGCTACAGTGGA | 532 | GAGGG | 1532 |
| | GAGGGCTTGGGGCGTACTCCG | | CTTGGG | |
| | GTGAGT | | GCGTA | |
| | | | CTCCG | |
| 19:19110944del | CTGGGCCCATGGAACGGTGGG | 533 | GGGTG | 1533 |
| G+ | GGGTGCGCTTGATTCTACTTCA | | CGCTTG | |
| | GGAGG | | ATTCTA | |
| | | | CTTC | |
| 2:1649188delG− | CCCCGCTCCTGGCCCGGGAGTC | 534 | GTCATT | 1534 |
| | ATTGGGGGGATCATGACAGAG | | GGGGG | |
| | AAG | | GATCAT | |
| | | | GACA | |
| 4:157221000del | CAAAGAAGGAAGAGGAGGAAA | 535 | AGGAA | 1535 |
| A+ | AGGAAAAAAAAGGGGTATATT | | AAAAA | |
| | GTGGAT | | AGGGG | |
| | | | TATATT | |
| 17:61482984del | CGTAGGGAGGGGGGAACGGAA | 536 | AGTGA | 1536 |
| C+ | ATAGTGATCCTCCCCCACCGAA | | TCCTCC | |
| | GAGGGGG | | CCCACC | |
| | | | GAAG | |
| 17:61482984del | CGTAGGGAGGGGGGAACGGAA | 537 | AATAG | 1537 |
| C+ | ATAGTGATCCTCCCCCACCGAA | | TGATCC | |
| | GAGG | | TCCCCC | |
| | | | ACCG | |
| 3:114339156del | CCTGGGGGGCCAGCGCGGGCA | 538 | CACCTG | 1538 |
| G− | CCTGGGGGTGTGCCTGCAGGG | | GGGGT | |
| | GGGT | | GTGCCT | |
| | | | GCAG | |
| 16:88624733del | CTGGGGGGGTCCTGGCTGGCCG | 539 | GCTCCT | 1539 |
| C+ | CTCCTTCCTCCTGGCTTCCCGA | | TCCTCC | |
| | GGGT | | TGGCTT | |
| | | | CCC | |
| 5:159203632del | CCAAGAAAAAAAAAAAGAAAA | 540 | AAAAA | 1540 |
| TC− | AAAAAACAACATGGCTGCAAA | | AAACA | |
| | GGAG | | ACATG | |
| | | | GCTGC | |
| | | | A | |
| 19:2038032delC− | CACTGCCCATATCTGTGGACTG | 541 | GCCCCT | 1541 |
| | CCCCTTCCAAAGACCCCTGGGG | | TCCAA | |
| | GGGT | | AGACC | |
| | | | CCTGG | |
| 11:72237704del | CCGAGGGCAGTCCCCGGGGGG | 542 | CTGCA | 1542 |
| C+ | CTGCAGCTCCAGGGGGCCTGG | | GCTCCA | |
| | GAGGAG | | GGGGG | |
| | | | CCTGG | |
| 2:1649188delG− | CCTGGCCCGGGAGTCATTGGGG | 543 | GGGGA | 1543 |
| | GGATCATGACAGAGAAGCAGG | | TCATGA | |
| | GGGG | | CAGAG | |
| | | | AAGCA | |
| 9:135487197del | CGCAGCCAGAGGGCCAGGGGG | 544 | CCCAC | 1544 |
| G+ | TCCCACATCTGGCCGAAGGGCT | | ATCTGG | |
| | TCGAGG | | CCGAA | |
| | | | GGGCT | |
| 22:21492040del | CAGAGTGCCAAGGGCCCAGAC | 545 | CCATGT | 1545 |
| C− | ACCATGTGAGCAGCAGCCAGC | | GAGCA | |
| | GGGGGGG | | GCAGC | |
| | | | CAGCG | |
| 3:127016548del | CCCGGGGGTGTCAGGCACCGG | 546 | GGATCT | 1546 |
| C+ | ATCTCACGGGAGTTCCTCCTGG | | CACGG | |
| | AGG | | GAGTTC | |
| | | | CTCC | |
| 1:156673012del | CTCTGCATCTACAGCAGGAGAG | 547 | GGGTG | 1547 |
| G− | GGTGCCTGAGGTGTGGGGGGA | | CCTGA | |
| | TGGGG | | GGTGT | |
| | | | GGGGG | |
| | | | G | |
| 20:49636246del | CACCGCCACCAAGAAAGCAGT | 548 | GTCCG | 1548 |
| A− | CCGATGAGATTTTTTTTGGAGG | | ATGAG | |
| | GGG | | ATTTTT | |
| | | | TTTGG | |
| 15:75207463del | CAGGGGGGCAGTGCTGGGGGG | 549 | GGTAC | 1549 |
| C+ | TACAGGGAACTGGACTGTCTCG | | AGGGA | |
| | GAT | | ACTGG | |
| | | | ACTGTC | |
| 19:11453958del | CAGTGGGGCTGGAAGCAGAAA | 550 | CAAAA | 1550 |
| T− | CAAAATGAAAAAAAAGGGGGG | | TGAAA | |
| | TGGGAG | | AAAAA | |
| | | | GGGGG | |
| | | | G | |
| 12:7089414delG− | CTCGGTCCTACCCCCTGACCTG | 551 | CGCTGC | 1551 |
| | CGCTGCAACTACAGCATCCGGG | | AACTA | |
| | TGGAG | | CAGCA | |
| | | | TCCGG | |
| 1:156673012del | CTCTGCATCTACAGCAGGAGAG | 552 | GTGCCT | 1552 |
| G− | GGTGCCTGAGGTGTGGGGGGA | | GAGGT | |
| | TGGGGGT | | GTGGG | |
| | | | GGGAT | |
| 10:100827567del | CGGGGGCGGCACTATAATAAT | 553 | AGGGG | 1553 |
| C+ | AAGGGGAACCTGGAAGTTAAC | | AACCT | |
| | ACAGGAG | | GGAAG | |
| | | | TTAACA | |
| 15:78104908del | CCCTGACCTCTTTTTTTTTTTCC | 554 | TTCCGC | 1554 |
| T+ | GCTCTGTCCTGGGTGACCAGGG | | TCTGTC | |
| | T | | CTGGGT | |
| | | | GAC | |
| 8:11305000delA+ | CATGGAGGGCGCTTCCCAGTAC | 555 | TAAGCT | 1555 |
| | TAAGCTATTACCACAAAAAAAT | | ATTACC | |
| | GGGAT | | ACAAA | |
| | | | AAAA | |
| 2:1649188delG− | CCTGGCCCGGGAGTCATTGGGG | 556 | GGGGG | 1556 |
| | GGATCATGACAGAGAAGCAGG | | ATCATG | |
| | GGG | | ACAGA | |
| | | | GAAGC | |
| 11:72237704del | CCGAGGGCAGTCCCCGGGGGG | 557 | GGCTG | 1557 |
| C+ | CTGCAGCTCCAGGGGGCCTGG | | CAGCTC | |
| | GAGG | | CAGGG | |
| | | | GGCCT | |
| 1:225403186del | CACTGACAGGGTCTGTACTTTT | 558 | TTTTCT | 1558 |
| A− | TTTTTCTTTTTGAGTCAGGACTA | | TTTTGA | |
| | TGGAG | | GTCAG | |
| | | | GACT | |
| 4:62071046delT+ | CTCAGACTTTTTTTTTTTAATGG | 559 | TGGGA | 1559 |
| | GATTTTTAGGTCAGCCCAGGGG | | TTTTTA | |
| | AG | | GGTCA | |
| | | | GCCCA | |
| 14:93241685del | CTCAGCCTCCATAAATCTAATG | 560 | TCATTT | 1560 |
| A− | TCATTTTTTTTTAATAAGTCCTA | | TTTTTT | |
| | GGAG | | AATAA | |
| | | | GTCC | |
| 9:89377969delA− | CAAGGAATAAAGTTAAAAAAA | 56 | AAAAA | 1561 |
| | AAAAAGAAAAAGAAAAAAGGT | | AAGAA | |
| | GAGT | | AAAGA | |
| | | | AAAAA | |
| | | | G | |
| 12:107544001del | CAGGGCCTCGGGCTCCCAGTAC | 562 | GTGCCC | 1562 |
| C+ | AGTGCCCCCTGCTTGTGGTGCA | | CCTGCT | |
| | GGGGGT | | TGTGGT | |
| | | | GCA | |
| 19:42334146del | CAGTGCCAGCCACCGGGTGTGT | 563 | GTGTGC | 1563 |
| G+ | GTGCCTGCGAGCCGGGCTGGG | | CTGCG | |
| | GGGT | | AGCCG | |
| | | | GGCTG | |
| 8:10726136delG− | CATGGAGACGCCGGGGGACTG | 564 | TGGCC | 1564 |
| | GCCAAGGGCCAGGGAGCCCAG | | AAGGG | |
| | GGGT | | CCAGG | |
| | | | GAGCC | |
| | | | C | |
| 15:42450759del | CCAGGGGAAAAAAATTTGTTTT | 565 | TGGTGT | 1565 |
| A− | GGTGTTTTTTGAGATATTCTTTG | | TTTTTG | |
| | GAT | | AGATA | |
| | | | TTCT | |
| 20:23085832del | CTGGGTGGGCGGGGGGAGGAC | 566 | ACGCCT | 1566 |
| C− | ACGCCTTACTCTAACTGGCACA | | TACTCT | |
| | AGGAG | | AACTG | |
| | | | GCAC | |
| 16:88624733del | CTGGGGGGGTCCTGGCTGGCCG | 567 | CCGCTC | 1567 |
| C+ | CTCCTTCCTCCTGGCTTCCCGA | | CTTCCT | |
| | GG | | CCTGGC | |
| | | | TTC | |
| 1:11024277delT+ | CATTGGCCAAAGTGAAAATTTT | 568 | TTTTTC | 1568 |
| | TTTTTTCTTTTGAAATCTAGTTT | | TTTTGA | |
| | TGAAT | | AATCTA | |
| | | | GTT | |
| 1:160371891del | CTGTGTGTCACTAGAGAAAAA | 569 | AAAAA | 1569 |
| A+ | AAAAACAAAAACCTAGATTCC | | AACAA | |
| | GGAT | | AAACC | |
| | | | TAGATT | |
| 10:29471187del | CCGAGTGCACCACCATCCCCCC | 570 | CTGGA | 1570 |
| C− | TGGAAACACTGCAGGAAACAG | | AACAC | |
| | GGGGG | | TGCAG | |
| | | | GAAAC | |
| | | | A | |
| 3:127016548del | CGCTGCCAGGGCTCTGCCCGGG | 571 | GGTGTC | 1571 |
| C+ | GGTGTCAGGCACCGGATCTCAC | | AGGCA | |
| | GGGAG | | CCGGA | |
| | | | TCTCA | |
| 7:50463377delT− | CATGGAACCAAGTGGATTTTTT | 572 | TTTGGC | 1572 |
| | GGCACTGTTTATTCTTTGCAGA | | ACTGTT | |
| | AG | | TATTCT | |
| | | | TTG | |
| 15:44711583del | CCGTGGCCTTAGCTGTGCTCGC | 573 | GCGCT | 1573 |
| CT+ | GCTATCTCTCTTTCTGGCCTGG | | ATCTCT | |
| | AGG | | CTTTCT | |
| | | | GGCC | |
| 22:21492040del | CAGAGTGCCAAGGGCCCAGAC | 574 | ACCAT | 1574 |
| C− | ACCATGTGAGCAGCAGCCAGC | | GTGAG | |
| | GGGGGG | | CAGCA | |
| | | | GCCAG | |
| | | | C | |
| 2:44209432delA+ | CCTGGGCGACACACCAAGGCT | 575 | GTCTCA | 1575 |
| | CTGTCTCAAAAAAAAAAAATTT | | AAAAA | |
| | AGAGAGG | | AAAAA | |
| | | | ATTTA | |
| 15:82436430del | CTAAGAGCGGGTCAAGAAATT | 576 | AAAAA | 1576 |
| A+ | GAAAAAAAAAACAAAACATTT | | AAAAA | |
| | AAGGGGT | | CAAAA | |
| | | | CATTTA | |
| 22:21492040del | CAGAGTGCCAAGGGCCCAGAC | 577 | CATGTG | 1577 |
| C− | ACCATGTGAGCAGCAGCCAGC | | AGCAG | |
| | GGGGGGGG | | CAGCC | |
| | | | AGCGG | |
| 15:44711583del | CCGTGGCCTTAGCTGTGCTCGC | 578 | CGCGCT | 1578 |
| CT+ | GCTATCTCTCTTTCTGGCCTGG | | ATCTCT | |
| | AG | | CTTTCT | |
| | | | GGC | |
| 11:72237704del | CGAGGGCAGTCCCCGGGGGGC | 579 | CTGCA | 1579 |
| C+ | TGCAGCTCCAGGGGGCCTGGG | | GCTCCA | |
| | AGGAG | | GGGGG | |
| | | | CCTGG | |
| 22:21492040del | CAGAGTGCCAAGGGCCCAGAC | 580 | CACCAT | 1580 |
| C− | ACCATGTGAGCAGCAGCCAGC | | GTGAG | |
| | GGGGG | | CAGCA | |
| | | | GCCAG | |
| 20:32453753del | CTCTGCCTCTTGCCCTCCTTTTA | 581 | TTTAGT | 1581 |
| G− | GTCCCCCCCTACAGCTCTGGGA | | CCCCCC | |
| | G | | CTACA | |
| | | | GCTC | |
| 3:51380173delC+ | CCTTGCGCAGGGTCCGGGCAG | 582 | GGAGG | 1582 |
| | GGAGGGCTGGGGGGTATCACC | | GCTGG | |
| | CAGAGG | | GGGGT | |
| | | | ATCACC | |
| X:130056036del | CCTGGGGGGATCTGTCGTCCAA | 583 | CAAGC | 1583 |
| C+ | GCCTCCAGGCCTCTCGGCAGGG | | CTCCAG | |
| | GT | | GCCTCT | |
| | | | CGGC | |
| 10:29471187del | CAGGGAAAGGAGCCCCCCTGT | 584 | GTTTCC | 1584 |
| C− | TTCCTGCAGTGTTTCCAGGGGG | | TGCAGT | |
| | GAT | | GTTTCC | |
| | | | AGG | |
| 5:55164285delT+ | CACTGTCTCCAAAAAAAAATGT | 585 | TTAAA | 1585 |
| | TTAAAATGAGACCAAACCCTCA | | ATGAG | |
| | TGGAG | | ACCAA | |
| | | | ACCCTC | |
| 20:32434639del | CACTGCCATCGGAGGGGGGGT | 586 | GTGGC | 1586 |
| G+ | GGCCCGGGTGGAGGTGGCGGC | | CCGGG | |
| | GGGG | | TGGAG | |
| | | | GTGGC | |
| | | | G | |
| 1:16922012delC+ | CACAGCCTCCCCCCATGAGCTT | 587 | GGGCT | 1587 |
| | GGGGCTGGCGGGGGCACAGGA | | GGCGG | |
| | GGTGGAG | | GGGCA | |
| | | | CAGGA | |
| | | | G | |
| 12:57466292del | CACGGGGAGCGGAAGGAGTTC | 588 | GTGCC | 1588 |
| G+ | GTGTGCCACTGGGGGGCTGCTC | | ACTGG | |
| | CAGGGAG | | GGGGC | |
| | | | TGCTCC | |
| 3:51380173delC+ | CCTTGCGCAGGGTCCGGGCAG | 589 | AGGGC | 1589 |
| | GGAGGGCTGGGGGGTATCACC | | TGGGG | |
| | CAGAGGGT | | GGTATC | |
| | | | ACCCA | |
| 3:127016548del | CGCTGCCAGGGCTCTGCCCGGG | 590 | GTGTCA | 1590 |
| C+ | GGTGTCAGGCACCGGATCTCAC | | GGCAC | |
| | GGGAGT | | CGGAT | |
| | | | CTCAC | |
| 10:100827567del | CCGGGGGCGGCACTATAATAA | 591 | AGGGG | 1591 |
| C+ | TAAGGGGAACCTGGAAGTTAA | | AACCT | |
| | CACAGGAG | | GGAAG | |
| | | | TTAACA | |
| 10:29471187del | CCGAGTGCACCACCATCCCCCC | 592 | CCTGG | 1592 |
| C− | TGGAAACACTGCAGGAAACAG | | AAACA | |
| | GGGG | | CTGCA | |
| | | | GGAAA | |
| | | | C | |
| 19:47416569del | CCCTGACGCCCCATCCCTGGGG | 593 | GGGTT | 1593 |
| C− | TTGGTGGGGGGGTGCAGAGAG | | GGTGG | |
| | AAG | | GGGGG | |
| | | | TGCAG | |
| | | | A | |
| 8:10726136delG− | CCAGGGGTGGCTACAGTGGAG | 594 | GAGGG | 1594 |
| | AGGGCTTGGGGCGTACTCCGGT | | CTTGGG | |
| | GAGT | | GCGTA | |
| | | | CTCCG | |
| 11:105003284del | CAGAGAGCACAGCACCTCATC | 595 | ATGATT | 1595 |
| T− | ATGATTTTTTTACACAGTCTCA | | TTTTTA | |
| | GGAAT | | CACAG | |
| | | | TCTC | |
| 7:93131425delT− | CTTTGACTTCATTTTTTTCCACA | 596 | ACACA | 1596 |
| | CATCCCCACTGTGCCAGAGGGA | | TCCCCA | |
| | AT | | CTGTGC | |
| | | | CAGA | |
| 12:121804752del | CGAAGGGCCGGGCTGCGGCGG | 597 | GGCTG | 1597 |
| C+ | GGGCTGCTGGTGGTGGTGGTGG | | CTGGTG | |
| | GGGGGT | | GTGGT | |
| | | | GGTGG | |
| 17:50356606del | CCTTGAGGGCTGGCGTGCCGGG | 598 | GGGGG | 1598 |
| C+ | GGGTAGCTGCCATACAGGTGG | | GTAGCT | |
| | AAG | | GCCAT | |
| | | | ACAGG | |
| 18:36625553del | CGATGAGACCACGGAGCCACC | 599 | CCCAGT | 1599 |
| C+ | CCCAGTGGGTGCCGGGACCGG | | GGGTG | |
| | AGGAGG | | CCGGG | |
| | | | ACCGG | |
| 11:44265074del | CAGGGGGTGGGCATGAGGGGA | 600 | ATGCA | 1600 |
| G− | CATGCAGGCAGGCACCGGGTC | | GGCAG | |
| | GCAGGGG | | GCACC | |
| | | | GGGTC | |
| | | | G | |
| 4:62071046delT+ | CCTGGGCTGACCTAAAAATCCC | 601 | CCCATT | 1601 |
| | ATTAAAAAAAAAAAGTCTGAG | | AAAAA | |
| | AGT | | AAAAA | |
| | | | AGTCT | |
| 15:82436430del | CTAAGAGCGGGTCAAGAAATT | 602 | GAAAA | 1602 |
| A+ | GAAAAAAAAAACAAAACATTT | | AAAAA | |
| | AAGGGG | | ACAAA | |
| | | | ACATTT | |
| X:129805329del | CCGAGGCTTAATGGAAGAACT | 603 | TGGTTA | 1603 |
| T− | GGTTAGCATTTTTTTTTTTTGAG | | GCATTT | |
| | GGT | | TTTTTT | |
| | | | TTT | |
| X:21994597delC+ | CACAGAGAAATAAAAAGGAAC | 604 | ACAAA | 1604 |
| | AAAAATCACATTCTAATGGGG | | AATCA | |
| | GGGT | | CATTCT | |
| | | | AATGG | |
| 1:154963387del | CTAAGAGATGGTCAAAGGCAC | 605 | ACAAA | 1605 |
| G− | AAAGTTTAAACATGGGGGGGC | | GTTTAA | |
| | GGGT | | ACATG | |
| | | | GGGGG | |
| X:80929822delA+ | CCCTGTCTTGATTTTAGCATTTT | 606 | TTTTTC | 1606 |
| | TTTCCCAGTGTTAGGTGAAAAG | | CCAGT | |
| | GAT | | GTTAG | |
| | | | GTGAA | |
| 15:44711583del | CCAGGCCAGAAAGAGAGATAG | 607 | AGCGC | 1607 |
| CT+ | CGCGAGCACAGCTAAGGCCAC | | GAGCA | |
| | GGAG | | CAGCT | |
| | | | AAGGC | |
| | | | C | |
| 17:75494982del | CTGGGGCTGGAGGGGGGATCT | 608 | TCGGA | 1608 |
| C+ | CGGAGCCAGGCATGTCACCATT | | GCCAG | |
| | GGGT | | GCATGT | |
| | | | CACCA | |
| 17:58357800del | CGGAGGGACCCCCCGCCTTTTC | 609 | TTCCTC | 1609 |
| C− | CTCTGTGGGTGTCGGGCAGAGA | | TGTGG | |
| | GG | | GTGTCG | |
| | | | GGCA | |
| 1:204954905del | CGGGGATGGAAGTCCAGGCGG | 610 | GGGGG | 1610 |
| C+ | GGGTTGCACTGGAGCGTCAAA | | TTGCAC | |
| | GGAG | | TGGAG | |
| | | | CGTCA | |
| 2:44209432delA+ | CTGGGCGACACACCAAGGCTCT | 611 | GTCTCA | 1611 |
| | GTCTCAAAAAAAAAAAATTTA | | AAAAA | |
| | GAGAGG | | AAAAA | |
| | | | ATTTA | |
| 15:64675048del | CCTTGACTCCAGCCAAGGACAA | 612 | CAAGA | 1612 |
| A+ | GAAAAAGAAAGACAAAAAAAG | | AAAAG | |
| | AAG | | AAAGA | |
| | | | CAAAA | |
| | | | A | |
| 15:64675048del | CCTTGACTCCAGCCAAGGACAA | 613 | AAAAA | 1613 |
| A+ | GAAAAAGAAAGACAAAAAAAG | | GAAAG | |
| | AAGGAAT | | ACAAA | |
| | | | AAAAG | |
| | | | A | |
| X:37453358delC+ | CCTGGAGACAATCCACTGCTGT | 614 | GTCAA | 1614 |
| | CAAACACTTCATCTGGTGGGGG | | ACACTT | |
| | GGT | | CATCTG | |
| | | | GTGG | |
| 1:25563141delT+ | CCAAGACTGCACCCCCCCTTGA | 615 | GAGCT | 1615 |
| | AGAGCTGTGAGTCCTGACGGG | | GTGAG | |
| | GAAGGGG | | TCCTGA | |
| | | | CGGGG | |
| 12:57466292del | CGGGGAGCGGAAGGAGTTCGT | 616 | GTGCC | 1616 |
| G+ | GTGCCACTGGGGGGCTGCTCCA | | ACTGG | |
| | GGGAG | | GGGGC | |
| | | | TGCTCC | |
| 1:25563141delT+ | CCAAGACTGCACCCCCCCTTGA | 617 | GAAGA | 1617 |
| | AGAGCTGTGAGTCCTGACGGG | | GCTGTG | |
| | GAAG | | AGTCCT | |
| | | | GACG | |
| 5:177248268del | CCTTGAGTGCAGCTCCTCCACC | 618 | CCGCGT | 1618 |
| A+ | GCGTTCACCCTGCATTTTTTAG | | TCACCC | |
| | AGG | | TGCATT | |
| | | | TTT | |
| 10:29471187del | CCCCGAGTGCACCACCATCCCC | 619 | CCCTGG | 1619 |
| C− | CCTGGAAACACTGCAGGAAAC | | AAACA | |
| | AGGGG | | CTGCA | |
| | | | GGAAA | |
| 10:29471187del | CCCCGAGTGCACCACCATCCCC | 620 | CTGGA | 1620 |
| C− | CCTGGAAACACTGCAGGAAAC | | AACAC | |
| | AGGGGGG | | TGCAG | |
| | | | GAAAC | |
| | | | A | |
| 10:29471187del | CCCCGAGTGCACCACCATCCCC | 621 | CCTGG | 1621 |
| C− | CCTGGAAACACTGCAGGAAAC | | AAACA | |
| | AGGGGG | | CTGCA | |
| | | | GGAAA | |
| | | | C | |
| 22:21492040del | CCAAGGGCCCAGACACCATGT | 622 | GTGAG | 1622 |
| C− | GAGCAGCAGCCAGCGGGGGGG | | CAGCA | |
| | GGGG | | GCCAG | |
| | | | CGGGG | |
| | | | G | |
| 1:13392592delT+ | CTAGGACACAGGTGGGTTTTTT | 623 | TTGTTT | 1623 |
| | TGTTTTTTTGTTTTTTTTTGATG | | TTTTGT | |
| | GAG | | TTTTTT | |
| | | | TTG | |
| 19:4816453delG− | CAGTGACTGTGGAAAGGCTGCT | 624 | CTGGCT | 1624 |
| | GGCTGTGGGGAGGGCGGTGGG | | GTGGG | |
| | GGGT | | GAGGG | |
| | | | CGGTG | |
| 8:61499736delT+ | CAAAGACTTGAGAGATGCTTTT | 625 | TTTTTT | 1625 |
| | TTTTCCCCCAGTGAGGGGACTG | | CCCCCA | |
| | GAG | | GTGAG | |
| | | | GGGA | |
| 8:61499736delT+ | CAAAGACTTGAGAGATGCTTTT | 626 | TTTTTC | 1626 |
| | TTTTCCCCCAGTGAGGGGACTG | | CCCCA | |
| | GAGG | | GTGAG | |
| | | | GGGAC | |
| 19:35732101del | CAGGGGGCCAAAGGAGACACC | 627 | CCCCCA | 1627 |
| G+ | CCCAAGGGCCTCCGGGATGGC | | AGGGC | |
| | GAGT | | CTCCGG | |
| | | | GATG | |
| 11:31790769del | CGAGGTGCCCATTGGCTGACTG | 628 | TCATGT | 1628 |
| G− | TTCATGTGTGTCTGCATATGTG | | GTGTCT | |
| | GGGGGT | | GCATAT | |
| | | | GTG | |
| 3:48158363delT− | CCCAGGGACTACCTCGGCTTTT | 629 | TTAATT | 1629 |
| | AATTTAAAAAAAAAAAGAAGT | | TAAAA | |
| | GGGT | | AAAAA | |
| | | | AAGAA | |
| 11:44265074del | CCCAGAGCCAGGGGGGGGCA | 630 | GAGGG | 1630 |
| G− | TGAGGGGACATGCAGGCAGGC | | GACAT | |
| | ACCGGGT | | GCAGG | |
| | | | CAGGC | |
| | | | A | |
| 8:61499736delT+ | CAAAGACTTGAGAGATGCTTTT | 631 | TTTCCC | 1631 |
| | TTTTCCCCCAGTGAGGGGACTG | | CCAGT | |
| | GAGGAT | | GAGGG | |
| | | | GACTG | |
| 1:13392592delT+ | CTAGGACACAGGTGGGTTTTTT | 632 | TGTTTT | 1632 |
| | TGTTTTTTTGTTTTTTTTTGATG | | TTTGTT | |
| | GAGT | | TTTTTT | |
| | | | TGA | |
| 8:10726136delG− | CCGGGGGACTGGCCAAGGGCC | 633 | GGGAG | 1633 |
| | AGGGAGCCCAGGGGTGGCTAC | | CCCAG | |
| | AGTGGAG | | GGGTG | |
| | | | GCTAC | |
| | | | A | |
| 16:28836029del | CCCAGAGTCATGGGGGCCCCCC | 634 | CCCAA | 1634 |
| G+ | CAAGGCGCGGTGCCCCAGAGT | | GGCGC | |
| | GGGG | | GGTGC | |
| | | | CCCAG | |
| | | | A | |
| 1:16135704delG− | CCTGGAGCATCCCCCGCCGCAG | 635 | AGCAG | 1635 |
| | CAGAGCCGAGTGTGGAAGTAC | | AGCCG | |
| | GAGG | | AGTGT | |
| | | | GGAAG | |
| | | | T | |
| 15:44711583del | CGTGGCCTTAGCTGTGCTCGCG | 636 | GCGCT | 1636 |
| CT+ | CTATCTCTCTTTCTGGCCTGGA | | ATCTCT | |
| | GG | | CTTTCT | |
| | | | GGCC | |
| 17:58357800del | CTCGGAGGGACCCCCCGCCTTT | 637 | TTCCTC | 1637 |
| C− | TCCTCTGTGGGTGTCGGGCAGA | | TGTGG | |
| | GAGG | | GTGTCG | |
| | | | GGCA | |
| 11:558069delG− | CTCCGAGAGGGCCTGTGGTTGG | 638 | GGTGG | 1638 |
| | TGGTGGGGGGTGTCTTCTGCAG | | TGGGG | |
| | AAG | | GGTGTC | |
| | | | TTCTG | |
| 2:70964443delC+ | CCGCGACAGGGAAGGGAGCAC | 639 | CGTTGA | 1639 |
| | GTTGATGGGGGGTAGATCTGA | | TGGGG | |
| | GGGAG | | GGTAG | |
| | | | ATCTG | |
| 1:154582079del | CAGTGACTTAACAATATACATT | 640 | TCCTCA | 1640 |
| T− | CCTCATAAATAAAAAAAAACA | | TAAAT | |
| | AGAAT | | AAAAA | |
| | | | AAAAC | |
| 16:28836029del | CCCAGAGTCATGGGGGCCCCCC | 641 | CCAAG | 1641 |
| G+ | CAAGGCGCGGTGCCCCAGAGT | | GCGCG | |
| | GGGGT | | GTGCCC | |
| | | | CAGAG | |
| 7:10982799delA+ | CAGCGAGCCAAAAAATGGAAC | 642 | CTTCGA | 1642 |
| | CTTCGACGAAACCGACCACTTC | | CGAAA | |
| | TGGAT | | CCGAC | |
| | | | CACTT | |
| 14:50976115del | CCACGCCTTAAAAATTGACAGT | 643 | AGTTG | 1643 |
| T− | TGAAAAAAAAAGAGTGACCAG | | AAAAA | |
| | AGG | | AAAAG | |
| | | | AGTGA | |
| | | | C | |
| 20:4727794delT+ | CTCTGCCTCAAAAAAAAAGTAT | 644 | TAGAA | 1644 |
| | AGAAAAATGAGTAGAAAGCAT | | AAATG | |
| | TGAAT | | AGTAG | |
| | | | AAAGC | |
| | | | A | |
| 11:44265074del | CCCGGTGCCTGCCTGCATGTCC | 645 | CCTCAT | 1645 |
| G− | CCTCATGCCCACCCCCTGGCTC | | GCCCA | |
| | TGGGG | | CCCCCT | |
| | | | GGCT | |
| 1:160371891del | CCCTGTGTGTCACTAGAGAAAA | 646 | AAAAA | 1646 |
| A+ | AAAAAACAAAAACCTAGATTC | | AACAA | |
| | CGGAT | | AAACC | |
| | | | TAGATT | |
| 1:204259283del | CAGTGGGTGAATCTGCGCCGG | 647 | GTACCC | 1647 |
| C− | GGGTACCCCCGCCTGAAGACCT | | CCGCCT | |
| | TCGGAGG | | GAAGA | |
| | | | CCTT | |
| 2:70964443delC+ | CCGCGACAGGGAAGGGAGCAC | 648 | TGATG | 1648 |
| | GTTGATGGGGGGTAGATCTGA | | GGGGG | |
| | GGGAGAAG | | TAGATC | |
| | | | TGAGG | |
| 4:92304602delA+ | CCGTGACCTCAAACTCTTTGGA | 649 | GACTGT | 1649 |
| | CTGTTTGAAAAAAAAAAATTG | | TTGAA | |
| | GAAG | | AAAAA | |
| | | | AAAAT | |
| 4:62071046delT− | CCCTGGGCTGACCTAAAAATCC | 650 | CCCATT | 1650 |
| | CATTAAAAAAAAAAAGTCTGA | | AAAAA | |
| | GAGT | | AAAAA | |
| | | | AGTCT | |
| 19:35732101del | CCAGGGGAGGGCAGGGGGCCA | 651 | GGAGA | 1651 |
| G+ | AAGGAGACACCCCCAAGGGCC | | CACCCC | |
| | TCCGGGAT | | CAAGG | |
| | | | GCCTC | |
| X:80444260delT+ | CCTGGACTTTTCAAGCATTTTTT | 652 | TTTTTT | 1652 |
| | TTGACAATTAAATTGGGTTGGA | | GACAA | |
| | T | | TTAAAT | |
| | | | TGGG | |
| 1:16135704delG− | CTTAGCGTCTCCTGGAGCATCC | 653 | CCGCC | 1653 |
| | CCCGCCGCAGCAGAGCCGAGT | | GCAGC | |
| | GTGGAAG | | AGAGC | |
| | | | CGAGT | |
| | | | G | |
| 1:25563141delT+ | CCTGGGGGCCCGCCAAGACTG | 654 | TGCACC | 1654 |
| | CACCCCCCCTTGAAGAGCTGTG | | CCCCCT | |
| | AGT | | TGAAG | |
| | | | AGCT | |
| 1:225403186del | CAGGGTCTGTACTTTTTTTTTCT | 655 | TTTTGA | 1655 |
| A− | TTTTGAGTCAGGACTATGGAGC | | GTCAG | |
| | CGAGT | | GACTAT | |
| | | | GGAG | |
| 2:85544939delT− | CATGGTGTTGAGAGAAAAAAA | 656 | AAATCT | 1656 |
| | AAAATCTTTTAAAAGCTGCCAT | | TTTAAA | |
| | CTGAGG | | AGCTG | |
| | | | CCAT | |
| 12:57466292del | CCTGGAGCAGCCCCCCAGTGGC | 657 | CACGA | 1657 |
| G+ | ACACGAACTCCTTCCGCTCCCC | | ACTCCT | |
| | GTGGAT | | TCCGCT | |
| | | | CCCC | |
| 5:132815809del | CAAAGTGCTTAGACATTTTCAA | 658 | ATTTTT | 1658 |
| T+ | TTTTTTTTTGCTAAATACTTTGG | | TTTTGC | |
| | AAT | | TAAAT | |
| | | | ACTT | |
| 2:97083988delG− | CCTTGAGAAAGACAGGAGGTT | 659 | TCCTGA | 1659 |
| | TCCTGAATACACCGACACCTGG | | ATACA | |
| | GGGGT | | CCGAC | |
| | | | ACCTG | |
| X:63754409delT | CCCTGTCTCTGTCTGTGATTTTT | 660 | TTTTTT | 1660 |
| T− | TTTTTTCTCGGTGGCTCTCGGG | | TTTCTC | |
| | AT | | GGTGG | |
| | | | CTCT | |
| 6:31958429delG− | CTAGGAGCCACCATGTTGGTGA | 661 | ACCCCC | 1661 |
| | TACCCCCGGACTGAGCGAGGA | | GGACT | |
| | AGAGGAG | | GAGCG | |
| | | | AGGAA | |
| 6:31958429delG− | CTAGGAGCCACCATGTTGGTGA | 662 | ATACCC | 1662 |
| | TACCCCCGGACTGAGCGAGGA | | CCGGA | |
| | AGAGG | | CTGAG | |
| | | | CGAGG | |
| 1:1354730delG− | CCCGGGGGGCGGCTCCGCGTG | 663 | TGGGG | 1663 |
| | GGGTTCGGCGACCGTCAGGTG | | TTCGGC | |
| | GAAG | | GACCG | |
| | | | TCAGG | |
| 20:32453753del | CCCAGAGCTGTAGGGGGGGAC | 664 | CTAAA | 1664 |
| G− | TAAAAGGAGGGCAAGAGGCAG | | AGGAG | |
| | AGGGT | | GGCAA | |
| | | | GAGGC | |
| | | | A | |
| X:63754409delT | CCCTGTCTCTGTCTGTGATTTTT | 665 | TTTTTT | 1665 |
| | TTTTTTCTCGGTGGCTCTCGGG | | TTTCTC | |
| | AT | | GGTGG | |
| | | | CTCT | |
| 11:18210242del | CGAAGGCCGAAAAAAAAAGAC | 666 | ATTGCT | 1666 |
| T+ | ATTGCTGAGTCCATTCTGGAAA | | GAGTC | |
| | AGAAT | | CATTCT | |
| | | | GGAA | |
| 1:204259283del | CAGTGGGTGAATCTGCGCCGG | 667 | GGTAC | 1667 |
| C− | GGGTACCCCCGCCTGAAGACCT | | CCCCGC | |
| | TCGGAG | | CTGAA | |
| | | | GACCT | |
| 6:111661353del | CAAAGAGGCCACTTTTGGAAA | 668 | AATAA | 1668 |
| T− | ATAATACTTTTTTTTTTTAGTTG | | TACTTT | |
| | AAT | | TTTTTT | |
| | | | TTAG | |
| 12:49040709del | CTTGGAGGAGAAGGTGCCAAA | 669 | AAGCC | 1669 |
| G− | GCCTGGGCAGGGGTGGCTCCTG | | TGGGC | |
| | GGG | | AGGGG | |
| | | | TGGCTC | |
| 3:42908691delT+ | CAGTGTCTTCAGGGGTAGGAG | 670 | GGGGA | 1670 |
| | GGGAAAAAACGGAAATAACTA | | AAAAA | |
| | GGAAG | | CGGAA | |
| | | | ATAACT | |
| 18:36625553del | CGATGAGACCACGGAGCCACC | 671 | CCCCA | 1671 |
| C+ | CCCAGTGGGTGCCGGGACCGG | | GTGGG | |
| | AGGAG | | TGCCG | |
| | | | GGACC | |
| | | | G | |
| 19:45052871del | CTGGGAGGTGGCTTACATGGTG | 672 | GGTGA | 1672 |
| C+ | GTGAGCAGAGGGGGGTGTAGT | | GCAGA | |
| | CGGGG | | GGGGG | |
| | | | GTGTA | |
| | | | G | |
| 2:131263505del | CTAAGAGAAAAGAAATATTTG | 673 | GGATA | 1673 |
| A+ | GAGGATATTGAAAGTGTGAAA | | TTGAA | |
| | AAAAGAAT | | AGTGT | |
| | | | GAAAA | |
| | | | A | |
| 10:29471187del | CCGAGTGCACCACCATCCCCCC | 674 | CCCTGG | 1674 |
| C− | TGGAAACACTGCAGGAAACAG | | AAACA | |
| | GGG | | CTGCA | |
| | | | GGAAA | |
| 19:45052871del | CTGGGAGGTGGCTTACATGGTG | 675 | TGAGC | 1675 |
| C+ | GTGAGCAGAGGGGGGTGTAGT | | AGAGG | |
| | CGGGGAT | | GGGGT | |
| | | | GTAGTC | |

Insertions

| | | | | |
|---|---|---|---|---|
| 3:195781031_ | CTGAGGAAAAGCTGGTGACAG | 676 | GGAAG | 1676 |
| 195781032insACC | GAAGAGGGGTGGCGTGACCTG | | AGGGG | |
| GGTGGATGCC | TGGAT | | TGGCGT | |
| GAGGAAGCGT | | | GACCT | |
| CGGTGACAGG | | | | |
| AAGAGGGGT | | | | |
| GGTGTCACCT | | | | |
| GTGGATACTG | | | | |
| AGGAAAAGCT | | | | |
| GGTGACAGGA | | | | |
| AGAGGGGTG | | | | |
| GCGTGACCTG | | | | |
| TGGATACTGA | | | | |
| GGAAGTGTCG | | | | |
| GTGACAGGAA | | | | |
| GAGTCGTGGT | | | | |
| GTC- | | | | |
| 3:195781031_ | CGAGGAAGCGTCGGTGACAGG | 677 | GGAAG | 1677 |
| 195781032insACC | AAGAGGGGTGGTGTCACCTGT | | AGGGG | |
| GGTGGATGCC | GGAT | | TGGTGT | |
| GAGGAAGCGT | | | CACCT | |
| CGGTGACAGG | | | | |
| AAGAGGGGT | | | | |
| GGTGTCACCT | | | | |
| GTGGATACTG | | | | |
| AGGAAAAGCT | | | | |
| GGTGACAGGA | | | | |
| AGAGGGGTG | | | | |
| GCGTGACCTG | | | | |
| TGGATACTGA | | | | |
| GGAAGTGTCG | | | | |
| GTGACAGGAA | | | | |
| GAGTCGTGGT | | | | |
| GTC- | | | | |
| 3:195781031_ | CCGAGGAAGCGTCGGTGACAG | 678 | GGAAG | 1678 |
| 195781032insACC | GAAGAGGGGTGGTGTCACCTG | | AGGGG | |
| GGTGGATGCC | TGGAT | | TGGTGT | |
| GAGGAAGCGT | | | CACCT | |
| CGGTGACAGG | | | | |
| AAGAGGGGT | | | | |
| GGTGTCACCT | | | | |
| GTGGATACTG | | | | |
| AGGAAAAGCT | | | | |
| GGTGACAGGA | | | | |
| AGAGGGGTG | | | | |
| GCGTGACCTG | | | | |
| TGGATACTGA | | | | |
| GGAAGTGTCG | | | | |
| GTGACAGGAA | | | | |
| GAGTCGTGGT | | | | |
| GTC- | | | | |
| 3:195781031_ | CTGAGGAAGTGTCGGTGACAG | 679 | GGAAG | 1679 |
| 195781032insACC | GAAGAGTCGTGGTGTCACCGGT | | AGTCGT | |
| GGTGGATGCC | GGAT | | GGTGTC | |
| GAGGAAGCGT | | | ACCG | |
| CGGTGACAGG | | | | |
| AAGAGGGGT | | | | |
| GGTGTCACCT | | | | |
| GTGGATACTG | | | | |
| AGGAAAAGCT | | | | |
| GGTGACAGGA | | | | |
| AGAGGGGTG | | | | |
| GCGTGACCTG | | | | |
| TGGATACTGA | | | | |
| GGAAGTGTCG | | | | |
| GTGACAGGAA | | | | |
| GAGTCGTGGT | | | | |
| GTC- | | | | |
| 6:167976333_ | CAGGGGGAATGACCCCCACTG | 680 | CTTCTC | 1680 |
| 167976334insA+ | TCTTCTCCTtCCCCACACACTGC | | CTtCCC | |
| | AGGGG | | CACAC | |
| | | | ACTG | |
| 6:167976333_ | CAGGGGGAATGACCCCCACTG | 681 | TTCTCC | 1681 |
| 167976334insA+ | TCTTCTCCTtCCCCACACACTGC | | TtCCCC | |
| | AGGGGG | | ACACA | |
| | | | CTGC | |
| 3:195781031_ | CGTGGTGTCACCGGTGGATGCT | 682 | TGAGG | 1682 |
| 195781032insACC | GAGGAAGCGCCGGTGACAGGA | | AAGCG | |
| GGTGGATGCC | AGAGT | | CCGGT | |
| GAGGAAGCGT | | | GACAG | |
| CGGTGACAGG | | | G | |
| AAGAGGGGT | | | | |
| GGTGTCACCT | | | | |
| GTGGATACTG | | | | |
| AGGAAAAGCT | | | | |
| GGTGACAGGA | | | | |
| AGAGGGGTG | | | | |
| GCGTGACCTG | | | | |
| TGGATACTGA | | | | |

GGAAGTGTCG
GTGACAGGAA
GAGTCGTGGT
GTC-
The + indicates the target sequence in on the coding strand in the genome. The −
indicates it is on the non-coding strand.
SEQ ID NO 683 (EGFR V769_D770insASV Target sequence)
CcacgctggcCACGCTGGCCATCACGTAGGCTTCCTGGAGGGAGGGAGAGG
SEQ ID NO: 1683: (EGFR guide RNA sequence)
CGTAGGCTTCCTGGAGGGAGG
SEQ ID NO: 1684: (EGFR guide RNA sequence)
TCACGTAGGCTTCCTGGAGGG
SEQ ID NO: 1685 (Muc4 guide RNA sequence)
GAAGAGTCGTGGTGTCACCG
SEQ ID NO: 1686 (EGFR L858R guide RNA sequence)
GATTTTGGGCgGGCCAAACTG
SEQ ID NO: 700
MGKSGIYQIKNTLNNKVYVGSAKDFEKRWKRHFKDLEKGCHSSIKLQRSFNKHGNVFE
CSI LEEIPYEKDLIIERENFWIKELNSKINGYNIA
LENGTH: 93
TYPE: AMINO ACID
FEATURE: I-TEVI DOMAIN
SEQ ID NO: 701
DATFGDTCSTHPLKEEIIKKRSETVKAKMLKLGPDGRKALYSKPGSKNGRWNPETHKFC
KCGVRIQTSAYTCSKCRN
LENGTH: 77
TYPE: AMINO ACID
FEATURE: LINKER DOMAIN
SEQ ID NO: 702 [I-TEVI WT NUCLEASE DOMAIN AND LINKER DOMAIN]
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRN
SEQ ID NO: 703
DATFGDTCSTHPLKEEIIKKRSETVKAKMLKLGPDGRKALYSKPGSKNGRWNPETHKFC
KCGVRIQTSAYTCSKCRNGGSGGS
LENGTH: 83
TYPE: AMINO ACID
FEATURE: LINKER DOMAIN with GGSGGS
SEQ ID NO: 704 [I-TEVI WT NUCLEASE DOMAIN AND LINKER DOMAIN with
GGSGGS]
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNGGSGGS
SEQ ID NO: 710
MKRNYILGLDIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
LENGTH: 1,053
TYPE: AMINO ACID
FEATURE: Staphylococcus aureus Cas9
SEQ ID NO: 711
MDKKYSIGLDIGTNSVGWAVITDEYKVPSKKFKVLGNTDRHSIKKNLIGALLFDSGETA
E
ATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLSDA
ILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
LENGTH: 1,368
TYPE: AMINO ACID
FEATURE: Streptococcus pyogenes Cas9
SEQ ID NO: 712
MAAFKPNPINYILGLDIGIASVGWAMVEIDEEENPIRLIDLGVRVFERAEVPKTGDSLAM
ARRLARSVRRLTRRRAHRLLRARRLLKREGVLQAADFDENGLIKSLPNTPWQLRAAAL
DRKLTPLEWSAVLLHLIKHRGYLSQRKNEGETADKELGALLKGVANNAHALQTGDFRT
PAELALNKFEKESGHIRNQRGDYSHTFSRKDLQAELILLFEKQKEFGNPHVSGGLKEGIE
TLLMTQRPALSGDAVQKMLGHCTFEPAEPKAAKNTYTAERFIWLTKLNNLRILEQGSER
PLTDTERATLMDEPYRKSKLTYAQARKLLGLEDTAFFKGLRYGKDNAEASTLMEMKA
YHAISRALEKEGLKDKKSPLNLSSELQDEIGTAFSLFKTDEDITGRLKDRVQPEILEALLK
HISFDKFVQISLKALRRIVPLMEQGKRYDEACAEIYGDHYGKKNTEEKIYLPPIPADKIRN
PVVLRALSQARKVINGVVRRYGSPARIHIETAREVGKSFKDRKEIEKRQEENRKDREKA
AAKFREYFPNFVGEPKSKDILKLRLYEQQHGKCLYSGKEINLVRLNEKGYVEIDHALPFS
RTWDDSFNNKVLVLGSENQNKGNQTPYEYFNGKDNSREWQEFKARVETSRFPRSKKQ
RILLQKFDEDGFKECNLNDTRYVNRFLCQFVADHILLTGKGKRRVFASNGQITNLLRGF
WGLRKVRAENDRHHALDAVVVACSTVAMQQKITRFVRYKEMNAFDGKTIDKETGKVL
HQKTHFPQPWEFFAQEVMIRVFGKPDGKPEFEEADTPEKLRTLLAEKLSSRPEAVHEYV
TPLFVSRAPNRKMSGAHKDTLRSAKRFVKHNEKISVKRVWLTEIKLADLENMVNYKNG
REIELYEALKARLEAYGGNAKQAFDPKDNPFYKKGGQLVKAVRVEKTQESGVLLNKK
NAYTIADNGDMVRVDVFCKVDKKGKNQYFIVPIYAWQVAENILPDIDCKGYRIDDSYTF
CFSLHKYDLIAFQKDEKSKVEFAYYINCDSSNGRFYLAWHDKGSKEQQFRISTQNLVLIQ
KYQVNELGKEIRPCRLKKRPPVR
LENGTH: 1082
TYPE: AMINO ACID
FEATURE: Neisseria meningitidis Cas9
SEQ ID NO: 713
MARILAFDIGISSIGWAFSENDELKDCGVRIFTKAENPKTGESLALPRRLARSARKRLARR
KARLNHLKHLIANEFKLNYEDYQSFDESLAKAYKGSLISPYELRFRALNELLSKQDFAR
VILHIAKRRGYDDIKNSDDKEKGAILKAIKQNEEKLANYQSVGEYLYKEYFQKFKENSK
EFTNVRNKKESYERCIAQSFLKDGLKLIFKKQREFGFSFSKKFEEEVLSVAFYKRALKDF
SHLVGNCSFFTDEKRAPKNSPLAFMFVALTRIINLLNNLKNTEGILYTKDDLNTLLNEVL
KNGTLTYKQTKKLLGLSDDYEFKREKGTYFIEFKKYKEFIKALGEHNLSQDDLNEIAKDI
TLIKDEIKLKKALAKYDLNQNQIDSLSKLEFKDHLNISFKALKLITPLMLEGKKYDEACN
ELNLKVAINEDKKDFLPAFNETYYKDEVTNPVVLRAIKEYRKVLNALLKKYGKVHKINI
ELAREVGKNHSQRAKIEKEQNENYKAKKDAELECEKLGLKINSKNILKLRLFKEQKEFC
AYSGEKIKISDLQDEKMLEIDHIYPYSRSFDDSYMNKVLVFTKQNQEKLNKTPFEAFGN
DSAKWQKIEVLAKNLPTKKQKRILDKNYKDKEQKDFKDRNLNDTRYIARLVLNYTKDY
LDFLPLSDDENTKLNDTQKGSKVHVEAKSGMLTSALRHTWGFSTKDRNNHLHHAIDAV
IIAYANNSIVKAFSDFKKEQESNSAELYAKKISELDYKNKRKFFEPFSGFRQKVLDKIDEI
FVSKPERKKPSGALHEETFRKEEEFYQSYGGKEGVLKALELGKIRKVNGKIVKNGDMFR
VDIFKHKKTNKFYAVPIYTMDFALKVLPNKAVARSKKGEIKDWILMDENYEFCFSLYK
DSLILIQTKDMQEPEFVYYNAFTSSTVSLIVSKHDNKFETLSKNQKILFKNANEKEVIAKS
IGIQNLKVFEKYIVSALGEVTKAEFRQREDFKK
LENGTH: 984
TYPE: AMINO ACID
FEATURE: Campylobacter jejuni Cas9
SEQ ID NO: 714
MTKKNYSIGLDIGTNSVGWAVITDDYKVPAKKMKVLGNTDKKYIKKNLLGALLFDSGE
TAEATRLKRTARRRYTRRKNRLRYLQEIFAEEMTKVDESFFYRLDESFLTTDEKDFERHP
IFGNKADEIKYHQEFPTIYHLRKHLADSSEKADLRLVYLALAHMIKFRGHFLIEGELNAE
NTDVQKIFADFVGVYDRTFDDSHLSEITVDAASILTEKISKSRRLENLIKYYPTEKKNTLF
GNLIALALGLQPNFKMNFKLSEDAKLQFSKDSYNEDLEELLGKIGDDYADLFTSAKNLY
DAILLSGILTVDDNSTKAPLSASMIKRYAEHHEDLEKLKEFIKANKSELYHDIFKDETKN
GYAGYIENGVKQDEFYKYLKNTLSKIAGSDYFLDKIEREDFLRKQRTFDNGSIPHQIHLQ
EMHAILRRQGDYYPFLKENQDRIEKILTFRIPYYVGPLARKDSRFSWAEYHSDEKITPWN
FDKVIDKEKSAEKFITRMTLNDLYLPEEKVLPKHSHVYETYAVYNELTKIKYVNEQGKD
SFFDSNMKQEIFDHVFKENRKVTKEKLLNYLNKEFPEYRIKDLIGLDKENKSFNASLGTY
HDLKKILDKAFLDDKVNEEVIEDIIKTLTLFEDKDMIHERLQKYSDIFTADQLKKLERRH
YTGWGRLSYKLINGIRNKENNKTILDYLIDDGSANRNFMQLINDDTLPFKQIIQKSQVVG
DVDDIEAVVHDLPGSPAIKKGILQSVKIVDELVKVMGDNPDNIVIEMARENQTTNRGRS
QSQQRLKKLQNSLKELGSNILNEEKPSYIEDKVENSHLQNDQLFLYYIQNGKDMYTGDE
LDIDHLSDYDIDHIIPQAFIKDDSIDNRVLTSSAKNRGKSDDVPSLDIVRARKAEWVRLY
KSGLISKRKFDNLTKAERGGLTEADKAGFIKRQLVETRQITKHVAQILDARFNTESDEND
KVIRDVKVITLKSNLVSQFRKDFEFYKVREINDYHHAHDAYLNAVVGTALLKKYPKLAS
EFVYGEYKKYDVHKLIAKSSDDHSEMGKATAKYFFYSNLMNFFKRVIRYSNGKVIVRP
VVEYSKDTEDIAWDKKSNFRTICKVLSYPQVNIVKKVETQTGGFSKESILPKGDSDKLIP
RKTKKAYWDTKKYGGFDSPTVAYSVFVVADVEKGKAKKLKTVKELVGISIMERSFFEE
NPVEFLENKGYHNIREDKLIKLPKYSLFEFEGGKRRLLASASELQKGNEMVIPGHLVKLL
YHAQRINSFNSTKYLDYVSAHKKEFEKVLSCVEDFANLYVDVEKNLSKIRAVADSMDN
FSIEEISNSFINLLTLTALGAPADFNFLGEKIPRKRYTSTKECLNATLIHQSITGLYETRIDLS
KIGEE
LENGTH: 1377
TYPE: AMINO ACID
FEATURE: Streptococcus pasteurianus Cas9
SEQ ID NO: 715
MKYTLGLDVGIASVGWAVIDKDNNKIIDLGVRCFDKAEESKTGESLATARRIARGMRRR
ISRRSQRLRLVKKLFVQYEIIKDSSEFNRIFDTSRDGWKDPWELRYNALSRILKPYELVQV
LTHITKRRGFKSNRKEDLSTTKEGVVITSIKNNSEMLRTKNYRTIGEMIFMETPENSNKR
NKVDEYIHTIAREDLLNEIKYIFSIQRKLGSPFVTEKLEHDFLNIWEFQRPFASGDSILSKV
GKCTLLKEELRAPTSCYTSEYFGLLQSINNLVLVEDNNTLTLNNDQRAKIIEYAHFKNEI
KYSEIRKLLDIEPEILFKAHNLTHKNPSGNNESKKFYEMKSYHKLKSTLPTDIWGKLHSN
KESLDNLFYCLTVYKNDNEIKDYLQANNLDYLIEYIAKLPTFNKFKHLSLVAMKRIIPFM
EKGYKYSDACNMAELDFTGSSKLEKCNKLTVEPIIENVTNPVVIRALTQARKVINAIIQK
YGLPYMVNIELAREAGMTRQDRDNLKKEHENNRKAREKISDLIRQNGRVASGLDILKW
RLWEDQGGRCAYSGKPIPVCDLLNDSLTQIDHIYPYSRSMDDSYMNKVLVLTDENQNK
RSYTPYEVWGSTEKWEDFEARIYSMHLPQSKEKRLLNRNFITKDLDSFISRNLNDTRYIS
RFLKNYIESYLQFSNDSPKSCVVCVNGQCTAQLRSRWGLNKNREESDLHHALDAAVIA
CADRKIIKEITNYYNERENHNYKVKYPLPWHSFRQDLMETLAGVFISRAPRRKITGPAHD
ETIRSPKHFNKGLTSVKIPLTTVTLEKLETMVKNTKGGISDKAVYNVLKNRLIEHNNKPL
KAFAEKIYKPLKNGTNGAIIRSIRVETPSYTGVFRNEGKGISDNSLMVRVDVFKKKDKYY
LVPIYVAHMIKKELPSKAIVPLKPESQWELIDSTHEFLFSLYQNDYLVIKTKKGITEGYYR
SCHRGTGSLSLMPHFANNKNVKIDIGVRTAISIEKYNVDILGNKSIVKGEPRRGMEKYNS
FKSN
LENGTH: 1021
TYPE: AMINO ACID
FEATURE: Clostridium cellulolyticum Cas9
SEQ ID NO: 716
MKYKIGLDIGITSIGWAVINLDIPRIEDLGVRIFDRAENPKTGESLALPRRLARSARRRLRR
RKHRLERIRRLFVREGILTKEELNKLFEKKHEIDVWQLRVEALDRKLNNDELARILLHLA
KRRGFRSNRKSERTNKENSTMLKHIEENQSILSSYRTVAEMVVKDPKFSLHKRNKEDNY
TNTVARDDLEREIKLIFAKQREYGNIVCTEAFEHEYISIWASQRPFASKDDIEKKVGFCTF
EPKEKRAPKATYTFQSFTVWEHINKLRLVSPGGIRALTDDERRLIYKQAFHKNKITFHDV
RTLLNLPDDTRFKGLLYDRNTTLKENEKVRFLELGAYHKIRKAIDSVYGKGAAKSFRPID
FDTFGYALTMFKDDTDIRSYLRNEYEQNGKRMENLADKVYDEELIEELLNLSFSKFGHL
SLKALRNILPYMEQGEVYSTACERAGYTFTGPKKKQKTVLLPNIPPIANPVVMRALTQA
RKVVNAIIKKYGSPVSIHIELARELSQSFDERRKMQKEQEGNRKKNETAIRQLVEYGLTL
NPTGLDIVKFKLWSEQNGKCAYSLQPIEIERLLEPGYTEVDHVIPYSRSLDDSYTNKVLV
LTKENREKGNRTPAEYLGLGSERWQQFETFVLINKQFSKKKRDRLLRLHYDENEENEF
KNRNLNDTRYISRFLANFIREHLKFADSDDKQKVYTVNGRITAHLRSRWNFNKNREESN
LHHAVDAAIVACTTPSDIARVTAFYQRREQNKELSKKTDPQFPQPWPHFADELQARLSK
NPKESIKALNLGNYDNEKLESLQPVFVSRMPKRSITGAAHQETLRRYIGIDERSGKIQTV
VKKKLSEIQLDKTGHFPMYGKESDPRTYEAIRQRLLEHNNDPKKAFQEPLYKPKKNGEL
GPIIRTIKIIDTTNQVIPLNDGKTVAYNSNIVRVDVFEKDGKYYCVPIYTIDMMKGILPNK
AIEPNKPYSEWKEMTEDYTFRFSLYPNDLIRIEFPREKTIKTAVGEEIKIKDLFAYYQTIDS
SNGGLSLVSHDNNFSLRSIGSRTLKRFEKYQVDVLGNIYKVRGEKRVGVASSSHSKAGE
TIRPL
LENGTH: 1082
TYPE: AMINO ACID
FEATURE: Geobacillus thermodenitrificansT1 Cas9
SEQ ID NO: 720 [Cas12a]
TQFEGFTNLYQVSKTLRFELIPQGKTLKHIQEQGFIEEDKARNDHYKELKPIIDRIYKTYA
DQCLQLVQLDWENLSAAIDSYRKEKTEETRNALIEEQATYRNAIHDYFIGRTDNLTDAIN
KRHAEIYKGLFKAELFNGKVLKQLGTVTTTEHENALLRSFDKFTTYFSGFYENRKNVFS
AEDISTAIPHRIVQDNFPKFKENCHIFTRLITAVPSLREHFENVKKAIGIFVSTSIEEVFSFPF
YNQLLTQTQIDLYNQLLGGISREAGTEKIKGLNEVLNLAIQKNDETAHIIASLPHRFIPLFK
QILSDRNTLSFILEEFKSDEEVIQSFCKYKTLLRNENVLETAEALFNELNSIDLTHIFISHKK
LETISSALCDHWDTLRNALYERRISELTGKITKSAKEKVQRSLKHEDINLQEIISAAGKEL
SEAFKQKTSEILSHAHAALDQPLPTTLKKQEEKEILKSQLDSLLGLYHLLDWFAVDESNE
VDPEFSARLTGIKLEMEPSLSFYNKARNYATKKPYSVEKFKLNFQMPTLASGWDVNKE
KNNGAILFVKNGLYYLGIMPKQKGRYKALSFEPTEKTSEGFDKMYYDYFPDAAKMIPK
CSTQLKAVTAHFQTHTTPILLSNNFIEPLEITKEIYDLNNPEKEPKKFQTAYAKKTGDQKG
YREALCKWIDFTRDFLSKYTKTTSIDLSSLRPSSQYKDLGEYYAELNPLLYHISFQRIAEK
EIMDAVETGKLYLFQIYNKDFAKGHHGKPNLHTLYWTGLFSPENLAKTSIKLNGQAELF
YRPKSRMKRMAHRLGEKMLNKKLKDQKTPIPDTLYQELYDYVNHRLSHDLSDEARAL
LPNVITKEVSHEIIKDRRFTSDKFFFHVPITLNYQAANSPSKFNQRVNAYLKEHPETPIIGI
DRGERNLIYITVIDSTGKILEQRSLNTIQQFDYQKKLDNREKERVAARQAWSVVGTIKDL
KQGYLSQVIHEIVDLMIHYQAVVVLENLNFGFKSKRTGIAEKAVYQQFEKMLIDKLNCL
VLKDYPAEKVGGVLNPYQLTDQFTSFAKMGTQSGFLFYVPAPYTSKIDPLTGFVDPFVW
KTIKNHESRKHFLEGFDFLHYDVKTGDFILHFKMNRNLSFQRGLPGFMPAWDIVFEKNE
TQFDAKGTPFIAGKRIVPVIENHRFTGRYRDLYPANELIALLEEKGIVERDGSNILPKLLEN
DDSHAIDTMVALIRSVLQMRNSNAATGEDYINSPVRDLNGVCFDSRFQNPEWPMDADA
NGAYHIALKGQLLLNHLKESKDLKLQNGISNQDWLAYIQELRN
SEQ ID NO: 721 [CasX]
QEIKRINKIRRRLVKDSNTKKAGKTGPMKTLLVRVMTPDLRERLENLRKKPENIPQPISN
TSRANLNKLLTDYTEMKKAILHVYWEEFQKDPVGLMSRVAQPAPKNIDQRKLIPVKDG
NERLTSSGFACSQCCQPLYVYKLEQVNDKGKPHTNYFGRCNVSEHERLILLSPHKPEAN
DELVTYSLGKFGQRALDFYSIHVTRESNHPVKPLEQIGGNSCASGPVGKALSDACMGAV
ASFLTKYQDIILEHQKVIKKNEKRLANLKDIASANGLAFPKITLPPQPHTKEGIEAYNNVV
AQIVIWVNLNLWQKLKIGRDEAKPLQRLKGFPSFPLVERQANEVDWWDMVCNVKKLI
NEKKEDGKVFWQNLAGYKRQEALLPYLSSEEDRKKGKKFARYQFGDLLLHLEKKHGE
DWGKVYDEAWERIDKKVEGLSKHIKLEEERRSEDAQSKAALTDWLRAKASFVIEGLKE
ADKDEFCRCELKLQKWYGDLRGKPFAIEAENSILDISGFSKQYNCAFIWQKDGVKKLNL
YLIINYFKGGKLRFKKIKPEAFEANRFYTVINKKSGEIVPMEVNFNFDDPNLIILPLAFGKR
QGREFIWNDLLSLETGSLKLANGRVIEKTLYNRRTRQDEPALFVALTFERREVLDSSNIK
PMNLIGIDRGENIPAVIALTDPEGCPLSRFKDSLGNPTHILRIGESYKEKQRTIQAAKEVEQ
RRAGGYSRKYASKAKNLADDMVRNTARDLLYYAVTQDAMLIFENLSRGFGRQGKRTF
MAERQYTRMEDWLTAKLAYEGLPSKTYLSKTLAQYTSKTCSNCGFTITSADYDRVLEK
LKKTATGWMTTINGKELKVEGQITYYNRYKRQNVVKDLSVELDRLSEESVNNDISSWT
KGRSGEALSLLKKRFSHRPVQEKFVCLNCGFETHADEQAALNIARSWLFLRSQEYKKYQ
TNKTTGNTDKRAFVETWQSFYRKKLKEVWKPAV
SEQ ID NO: 730 [I-TEVI WT NUCLEASE DOMAIN, LINKER DOMAIN, and Staphylococcus
aureus Cas9]
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNMKRNYILGL
DIGITSVGYGIIDYETRDVIDAGVRLFKEANVENNEGRRSKRGARRLKRRRRHRIQRVKK
LLFDYNLLTDHSELSGINPYEARVKGLSQKLSEEEFSAALLHLAKRRGVHNVNEVEEDT
GNELSTKEQISRNSKALEEKYVAELQLERLKKDGEVRGSINRFKTSDYVKEAKQLLKVQ
KAYHQLDQSFIDTYIDLLETRRTYYEGPGEGSPFGWKDIKEWYEMLMGHCTYFPEELRS
VKYAYNADLYNALNDLNNLVITRDENEKLEYYEKFQIIENVFKQKKKPTLKQIAKEILV
NEEDIKGYRVTSTGKPEFTNLKVYHDIKDITARKEIIENAELLDQIAKILTIYQSSEDIQEEL
TNLNSELTQEEIEQISNLKGYTGTHNLSLKAINLILDELWHTNDNQIAIFNRLKLVPKKVD
LSQQKEIPTTLVDDFILSPVVKRSFIQSIKVINAIIKKYGLPNDIIIELAREKNSKDAQKMIN
EMQKRNRQTNERIEEIIRTTGKENAKYLIEKIKLHDMQEGKCLYSLEAIPLEDLLNNPFNY
EVDHIIPRSVSFDNSFNNKVLVKQEENSKKGNRTPFQYLSSSDSKISYETFKKHILNLAKG
KGRISKTKKEYLLEERDINRFSVQKDFINRNLVDTRYATRGLMNLLRSYFRVNNLDVKV
KSINGGFTSFLRRKWKFKKERNKGYKHHAEDALIIANADFIFKEWKKLDKAKKVMENQ
MFEEKQAESMPEIETEQEYKEIFITPHQIKHIKDFKDYKYSHRVDKKPNRELINDTLYSTR
KDDKGNTLIVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNSRNKVVKLS
LKPYRFDVYLDNGVYKFVTVKNLDVIKKENYYEVNSKCYEEAKKLKKISNQAEFIASFY
NNDLIKINGELYRVIGVNNDLLNRIEVNMIDITYREYLENMNDKRPPRIIKTIASKTQSIKK
YSTDILGNLYEVKSKKHPQIIKKG
SEQ ID NO: 750 [I-TEVI WT NUCLEASE DOMAIN, LINKER DOMAIN with GGSGGS, and
Staphylococcus aureus Cas9]
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNGGSGGSMK
RNYILGLDIGITSVGYGIIDYETRDVIDAGVRLFKEANVENNEGRRSKRGARRLKRRRRH
RIQRVKKLLFDYNLLTDHSELSGINPYEARVKGLSQKLSEEEFSAALLHLAKRRGVHNV
NEVEEDTGNELSTKEQISRNSKALEEKYVAELQLERLKKDGEVRGSINRFKTSDYVKEA
KQLLKVQKAYHQLDQSFIDTYIDLLETRRTYYEGPGEGSPFGWKDIKEWYEMLMGHCT
YFPEELRSVKYAYNADLYNALNDLNNLVITRDENEKLEYYEKFQIIENVFKQKKKPTLK
QIAKEILVNEEDIKGYRVTSTGKPEFTNLKVYHDIKDITARKEIIENAELLDQIAKILTIYQS
SEDIQEELTNLNSELTQEEIEQISNLKGYTGTHNLSLKAINLILDELWHTNDNQIAIFNRLK
LVPKKVDLSQQKEIPTTLVDDFILSPVVKRSFIQSIKVINAIIKKYGLPNDIIIELAREKNSK
DAQKMINEMQKRNRQTNERIEEIIRTTGKENAKYLIEKIKLHDMQEGKCLYSLEAIPLED
LLNNPFNYEVDHIIPRSVSFDNSFNNKVLVKQEENSKKGNRTPFQYLSSSDSKISYETFKK
HILNLAKGKGRISKTKKEYLLEERDINRFSVQKDFINRNLVDTRYATRGLMNLLRSYFRV
NNLDVKVKSINGGFTSFLRRKWKFKKERNKGYKHHAEDALIIANADFIFKEWKKLDKA
KKVMENQMFEEKQAESMPEIETEQEYKEIFITPHQIKHIKDFKDYKYSHRVDKKPNRELI
NDTLYSTRKDDKGNTLIVNNLNGLYDKDNDKLKKLINKSPEKLLMYHHDPQTYQKLKL
IMEQYGDEKNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEAKKLKKISN
QAEFIASFYNNDLIKINGELYRVIGVNNDLLNRIEVNMIDITYREYLENMNDKRPPRIIKTI
ASKTQSIKKYSTDILGNLYEVKSKKHPQIIKKG
SEQ ID NO: 731 [I-TEVI WT NUCLEASE DOMAIN, LINKER DOMAIN, and Streptococcus
pyogenes Cas9]
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNMDKKYSIGL
DIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLFGNLIALSLGLT
PNFKSNFDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFFDQSKNGYAGYIDGGA
SQEEFYKFIKPILEKMDGTEELLVKLNREDLLRKQRTFDNGSIPHQIHLGELHAILRRQED
FYPFLKDNREKIEKILTFRIPYYVGPLARGNSRFAWMTRKSEETITPWNFEEVVDKGASA
QSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRYTGWGRLSRK
LINGIRDKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRERMKRI
EEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVR
KMIAKSEQEIGKATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKKYGGFDSPTV
AYSVLVVAKVEKGKSKKLKSVKELLGITIMERSSFEKNPIDFLEAKGYKEVKKDLIIKLP
KYSLFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGD
SEQ ID NO: 751 [I-TEVI WT NUCLEASE DOMAIN, LINKER DOMAIN with GGSGGS, and
Streptococcus pyogenes Cas9]
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNGGSGGSMD
KKYSIGLDIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDYDV
DHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKKYGG
FDSPTVAYSVLVVAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDLSQLGGD
SEQ ID NO: 732 [I-TEVI WT NUCLEASE DOMAIN, LINKER DOMAIN, and Neisseria
meningitidis Cas9]
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNMAAFKPNPI
NYILGLDIGIASVGWAMVEIDEEENPIRLIDLGVRVFERAEVPKTGDSLAMARRLARSVR
RLTRRRAHRLLRARRLLKREGVLQAADFDENGLIKSLPNTPWQLRAAALDRKLTPLEW
SAVLLHLIKHRGYLSQRKNEGETADKELGALLKGVANNAHALQTGDFRTPAELALNKF
EKESGHIRNQRGDYSHTFSRKDLQAELILLFEKQKEFGNPHVSGGLKEGIETLLMTQRPA
LSGDAVQKMLGHCTFEPAEPKAAKNTYTAERFIWLTKLNNLRILEQGSERPLTDTERAT
LMDEPYRKSKLTYAQARKLLGLEDTAFFKGLRYGKDNAEASTLMEMKAYHAISRALEK
EGLKDKKSPLNLSSELQDEIGTAFSLFKTDEDITGRLKDRVQPEILEALLKHISFDKFVQIS
LKALRRIVPLMEQGKRYDEACAEIYGDHYGKKNTEEKIYLPPIPADKIRNPVVLRALSQA
RKVINGVVRRYGSPARIHIETAREVGKSFKDRKEIEKRQEENRKDREKAAAKFREYFPNF
VGEPKSKDILKLRLYEQQHGKCLYSGKEINLVRLNEKGYVEIDHALPFSRTWDDSFNNK
VLVLGSENQNKGNQTPYEYFNGKDNSREWQEFKARVETSRFPRSKKQRILLQKFDEDG
FKECNLNDTRYVNRFLCQFVADHILLTGKGKRRVFASNGQITNLLRGFWGLRKVRAEN
DRHHALDAVVVACSTVAMQQKITRFVRYKEMNAFDGKTIDKETGKVLHQKTHFPQPW
EFFAQEVMIRVFGKPDGKPEFEEADTPEKLRTLLAEKLSSRPEAVHEYVTPLFVSRAPNR
KMSGAHKDTLRSAKRFVKHNEKISVKRVWLTEIKLADLENMVNYKNGREIELYEALKA
RLEAYGGNAKQAFDPKDNPFYKKGGQLVKAVRVEKTQESGVLLNKKNAYTIADNGDM
VRVDVFCKVDKKGKNQYFIVPIYAWQVAENILPDIDCKGYRIDDSYTFCFSLHKYDLIAF
QKDEKSKVEFAYYINCDSSNGRFYLAWHDKGSKEQQFRISTQNLVLIQKYQVNELGKEI
RPCRLKKRPPVR
SEQ ID NO: 752 [I-TEVI WT NUCLEASE DOMAIN, LINKER DOMAIN with GGSGGS, and
Neisseria meningitidis Cas9]
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNGGSGGSMA
AFKPNPINYILGLDIGIASVGWAMVEIDEEENPIRLIDLGVRVFERAEVPKTGDSLAMARR
LARSVRRLTRRRAHRLLRARRLLKREGVLQAADFDENGLIKSLPNTPWQLRAAALDRK
LTPLEWSAVLLHLIKHRGYLSQRKNEGETADKELGALLKGVANNAHALQTGDFRTPAE
LALNKFEKESGHIRNQRGDYSHTFSRKDLQAELILLFEKQKEFGNPHVSGGLKEGIETLL
MTQRPALSGDAVQKMLGHCTFEPAEPKAAKNTYTAERFIWLTKLNNLRILEQGSERPLT
DTERATLMDEPYRKSKLTYAQARKLLGLEDTAFFKGLRYGKDNAEASTLMEMKAYHA
ISRALEKEGLKDKKSPLNLSSELQDEIGTAFSLFKTDEDITGRLKDRVQPEILEALLKHISF
DKFVQISLKALRRIVPLMEQGKRYDEACAEIYGDHYGKKNTEEKIYLPPIPADKIRNPVV
LRALSQARKVINGVVRRYGSPARIHIETAREVGKSFKDRKEIEKRQEENRKDREKAAAK
FREYFPNFVGEPKSKDILKLRLYEQQHGKCLYSGKEINLVRLNEKGYVEIDHALPFSRTW
DDSFNNKVLVLGSENQNKGNQTPYEYFNGKDNSREWQEFKARVETSRFPRSKKQRILL
QKFDEDGFKECNLNDTRYVNRFLCQFVADHILLTGKGKRRVFASNGQITNLLRGFWGL
RKVRAENDRHHALDAVVVACSTVAMQQKITRFVRYKEMNAFDGKTIDKETGKVLHQK
THFPQPWEFFAQEVMIRVFGKPDGKPEFEEADTPEKLRTLLAEKLSSRPEAVHEYVTPLF
VSRAPNRKMSGAHKDTLRSAKRFVKHNEKISVKRVWLTEIKLADLENMVNYKNGREIE
LYEALKARLEAYGGNAKQAFDPKDNPFYKKGGQLVKAVRVEKTQESGVLLNKKNAYT
IADNGDMVRVDVFCKVDKKGKNQYFIVPIYAWQVAENILPDIDCKGYRIDDSYTFCFSL
HKYDLIAFQKDEKSKVEFAYYINCDSSNGRFYLAWHDKGSKEQQFRISTQNLVLIQKYQ
VNELGKEIRPCRLKKRPPVR
SEQ ID NO: 733 [I-TEVI WT NUCLEASE DOMAIN, LINKER DOMAIN, and Campylobacter
jejuni Cas9]
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNMARILAFDI
GISSIGWAFSENDELKDCGVRIFTKAENPKTGESLALPRRLARSARKRLARRKARLNHLK
HLIANEFKLNYEDYQSFDESLAKAYKGSLISPYELRFRALNELLSKQDFARVILHIAKRR
GYDDIKNSDDKEKGAILKAIKQNEEKLANYQSVGEYLYKEYFQKFKENSKEFTNVRNK
KESYERCIAQSFLKDGLKLIFKKQREFGFSFSKKFEEEVLSVAFYKRALKDFSHLVGNCS
FFTDEKRAPKNSPLAFMFVALTRIINLLNNLKNTEGILYTKDDLNTLLNEVLKNGTLTYK
QTKKLLGLSDDYEFKREKGTYFIEFKKYKEFIKALGEHNLSQDDLNEIAKDITLIKDEIKL
KKALAKYDLNQNQIDSLSKLEFKDHLNISFKALKLITPLMLEGKKYDEACNELNLKVAI
NEDKKDFLPAFNETYYKDEVTNPVVLRAIKEYRKVLNALLKKYGKVHKINIELAREVG
KNHSQRAKIEKEQNENYKAKKDAELECEKLGLKINSKNILKLRLFKEQKEFCAYSGEKI
KISDLQDEKMLEIDHIYPYSRSFDDSYMNKVLVFTKQNQEKLNKTPFEAFGNDSAKWQ
KIEVLAKNLPTKKQKRILDKNYKDKEQKDFKDRNLNDTRYIARLVLNYTKDYLDFLPLS
DDENTKLNDTQKGSKVHVEAKSGMLTSALRHTWGFSTKDRNNHLHHAIDAVIIAYANN
SIVKAFSDFKKEQESNSAELYAKKISELDYKNKRKFFEPFSGFRQKVLDKIDEIFVSKPER
KKPSGALHEETFRKEEEFYQSYGGKEGVLKALELGKIRKVNGKIVKNGDMFRVDIFKHK
KTNKFYAVPIYTMDFALKVLPNKAVARSKKGEIKDWILMDENYEFCFSLYKDSLILIQTK
DMQEPEFVYYNAFTSSTVSLIVSKHDNKFETLSKNQKILFKNANEKEVIAKSIGIQNLKVF
EKYIVSALGEVTKAEFRQREDFKK
SEQ ID NO: 753 [I-TEVI WT NUCLEASE DOMAIN, LINKER DOMAIN with GGSGGS, and
Campylobacter jejuni Cas9]
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNGGSGGSMA
RILAFDIGISSIGWAFSENDELKDCGVRIFTKAENPKTGESLALPRRLARSARKRLARRKA
RLNHLKHLIANEFKLNYEDYQSFDESLAKAYKGSLISPYELRFRALNELLSKQDFARVIL
HIAKRRGYDDIKNSDDKEKGAILKAIKQNEEKLANYQSVGEYLYKEYFQKFKENSKEFT
NVRNKKESYERCIAQSFLKDGLKLIFKKQREFGFSFSKKFEEEVLSVAFYKRALKDFSHL
VGNCSFFTDEKRAPKNSPLAFMFVALTRIINLLNNLKNTEGILYTKDDLNTLLNEVLKNG
TLTYKQTKKLLGLSDDYEFKREKGTYFIEFKKYKEFIKALGEHNLSQDDLNEIAKDITLIK
DEIKLKKALAKYDLNQNQIDSLSKLEFKDHLNISFKALKLITPLMLEGKKYDEACNELNL
KVAINEDKKDFLPAFNETYYKDEVTNPVVLRAIKEYRKVLNALLKKYGKVHKINIELAR
EVGKNHSQRAKIEKEQNENYKAKKDAELECEKLGLKINSKNILKLRLFKEQKEFCAYSG
EKIKISDLQDEKMLEIDHIYPYSRSFDDSYMNKVLVFTKQNQEKLNKTPFEAFGNDSAK
WQKIEVLAKNLPTKKQKRILDKNYKDKEQKDFKDRNLNDTRYIARLVLNYTKDYLDFL
PLSDDENTKLNDTQKGSKVHVEAKSGMLTSALRHTWGFSTKDRNNHLHHAIDAVIIAY
ANNSIVKAFSDFKKEQESNSAELYAKKISELDYKNKRKFFEPFSGFRQKVLDKIDEIFVSK
PERKKPSGALHEETFRKEEEFYQSYGGKEGVLKALELGKIRKVNGKIVKNGDMFRVDIF
KHKKTNKFYAVPIYTMDFALKVLPNKAVARSKKGEIKDWILMDENYEFCFSLYKDSLIL
IQTKDMQEPEFVYYNAFTSSTVSLIVSKHDNKFETLSKNQKILFKNANEKEVIAKSIGIQN
LKVFEKYIVSALGEVTKAEFRQREDFKK
SEQ ID NO: 734 [I-TEVI WT NUCLEASE DOMAIN, LINKER DOMAIN, and Streptococcus
pasteurianus Cas9]
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNMTKKNYSIG
LDIGTNSVGWAVITDDYKVPAKKMKVLGNTDKKYIKKNLLGALLFDSGETAEATRLKR
TARRRYTRRKNRLRYLQEIFAEEMTKVDESFFYRLDESFLTTDEKDFERHPIFGNKADEI
KYHQEFPTIYHLRKHLADSSEKADLRLVYLALAHMIKFRGHFLIEGELNAENTDVQKIFA
DFVGVYDRTFDDSHLSEITVDAASILTEKISKSRRLENLIKYYPTEKKNTLFGNLIALALG
LQPNFKMNFKLSEDAKLQFSKDSYNEDLEELLGKIGDDYADLFTSAKNLYDAILLSGILT
VDDNSTKAPLSASMIKRYAEHHEDLEKLKEFIKANKSELYHDIFKDETKNGYAGYIENG
VKQDEFYKYLKNTLSKIAGSDYFLDKIEREDFLRKQRTFDNGSIPHQIHLQEMHAILRRQ
GDYYPFLKENQDRIEKILTFRIPYYVGPLARKDSRFSWAEYHSDEKITPWNFDKVIDKEK
SAEKFITRMTLNDLYLPEEKVLPKHSHVYETYAVYNELTKIKYVNEQGKDSFFDSNMKQ
EIFDHVFKENRKVTKEKLLNYLNKEFPEYRIKDLIGLDKENKSFNASLGTYHDLKKILDK
AFLDDKVNEEVIEDIIKTLTLFEDKDMIHERLQKYSDIFTADQLKKLERRHYTGWGRLSY
KLINGIRNKENNKTILDYLIDDGSANRNFMQLINDDTLPFKQIIQKSQVVGDVDDIEAVV
HDLPGSPAIKKGILQSVKIVDELVKVMGDNPDNIVIEMARENQTTNRGRSQSQQRLKKL
QNSLKELGSNILNEEKPSYIEDKVENSHLQNDQLFLYYIQNGKDMYTGDELDIDHLSDY
DIDHIIPQAFIKDDSIDNRVLTSSAKNRGKSDDVPSLDIVRARKAEWVRLYKSGLISKRKF
DNLTKAERGGLTEADKAGFIKRQLVETRQITKHVAQILDARFNTESDENDKVIRDVKVIT
LKSNLVSQFRKDFEFYKVREINDYHHAHDAYLNAVVGTALLKKYPKLASEFVYGEYKK
YDVHKLIAKSSDDHSEMGKATAKYFFYSNLMNFFKRVIRYSNGKVIVRPVVEYSKDTE
DIAWDKKSNFRTICKVLSYPQVNIVKKVETQTGGFSKESILPKGDSDKLIPRKTKKAYWD
TKKYGGFDSPTVAYSVFVVADVEKGKAKKLKTVKELVGISIMERSFFEENPVEFLENKG
YHNIREDKLIKLPKYSLFEFEGGKRRLLASASELQKGNEMVIPGHLVKLLYHAQRINSFN
STKYLDYVSAHKKEFEKVLSCVEDFANLYVDVEKNLSKIRAVADSMDNFSIEEISNSFIN
LLTLTALGAPADFNFLGEKIPRKRYTSTKECLNATLIHQSITGLYETRIDLSKIGEE
SEQ ID NO: 754 [I-TEVI WT NUCLEASE DOMAIN, LINKER DOMAIN with GGSGGS, and
Streptococcus pasteurianus Cas9]
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNGGSGGSMT
KKNYSIGLDIGTNSVGWAVITDDYKVPAKKMKVLGNTDKKYIKKNLLGALLFDSGETA
EATRLKRTARRRYTRRKNRLRYLQEIFAEEMTKVDESFFYRLDESFLTTDEKDFERHPIF
GNKADEIKYHQEFPTIYHLRKHLADSSEKADLRLVYLALAHMIKFRGHFLIEGELNAENT
DVQKIFADFVGVYDRTFDDSHLSEITVDAASILTEKISKSRRLENLIKYYPTEKKNTLFGN
LIALALGLQPNFKMNFKLSEDAKLQFSKDSYNEDLEELLGKIGDDYADLFTSAKNLYDA
ILLSGILTVDDNSTKAPLSASMIKRYAEHHEDLEKLKEFIKANKSELYHDIFKDETKNGY
AGYIENGVKQDEFYKYLKNTLSKIAGSDYFLDKIEREDFLRKQRTFDNGSIPHQIHLQEM
HAILRRQGDYYPFLKENQDRIEKILTFRIPYYVGPLARKDSRFSWAEYHSDEKITPWNFD
KVIDKEKSAEKFITRMTLNDLYLPEEKVLPKHSHVYETYAVYNELTKIKYVNEQGKDSF
FDSNMKQEIFDHVFKENRKVTKEKLLNYLNKEFPEYRIKDLIGLDKENKSFNASLGTYH
DLKKILDKAFLDDKVNEEVIEDIIKTLTLFEDKDMIHERLQKYSDIFTADQLKKLERRHY
TGWGRLSYKLINGIRNKENNKTILDYLIDDGSANRNFMQLINDDTLPFKQIIQKSQVVGD
VDDIEAVVHDLPGSPAIKKGILQSVKIVDELVKVMGDNPDNIVIEMARENQTTNRGRSQS
QQRLKKLQNSLKELGSNILNEEKPSYIEDKVENSHLQNDQLFLYYIQNGKDMYTGDELD
IDHLSDYDIDHIIPQAFIKDDSIDNRVLTSSAKNRGKSDDVPSLDIVRARKAEWVRLYKSG
LISKRKFDNLTKAERGGLTEADKAGFIKRQLVETRQITKHVAQILDARFNTESDENDKVI
RDVKVITLKSNLVSQFRKDFEFYKVREINDYHHAHDAYLNAVVGTALLKKYPKLASEF
VYGEYKKYDVHKLIAKSSDDHSEMGKATAKYFFYSNLMNFFKRVIRYSNGKVIVRPVV
EYSKDTEDIAWDKKSNFRTICKVLSYPQVNIVKKVETQTGGFSKESILPKGDSDKLIPRK
TKKAYWDTKKYGGFDSPTVAYSVFVVADVEKGKAKKLKTVKELVGISIMERSFFEENP
VEFLENKGYHNIREDKLIKLPKYSLFEFEGGKRRLLASASELQKGNEMVIPGHLVKLLYH
AQRINSFNSTKYLDYVSAHKKEFEKVLSCVEDFANLYVDVEKNLSKIRAVADSMDNFSI
EEISNSFINLLTLTALGAPADFNFLGEKIPRKRYTSTKECLNATLIHQSITGLYETRIDLSKI
GEE
SEQ ID NO: 735 [I-TEVI WT NUCLEASE DOMAIN, LINKER DOMAIN, and Clostridium
cellulolyticum Cas9]
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNMKYTLGLD
VGIASVGWAVIDKDNNKIIDLGVRCFDKAEESKTGESLATARRIARGMRRRISRRSQRLR
LVKKLFVQYEIIKDSSEFNRIFDTSRDGWKDPWELRYNALSRILKPYELVQVLTHITKRR
GFKSNRKEDLSTTKEGVVITSIKNNSEMLRTKNYRTIGEMIFMETPENSNKRNKVDEYIH
TIAREDLLNEIKYIFSIQRKLGSPFVTEKLEHDFLNIWEFQRPFASGDSILSKVGKCTLLKE
ELRAPTSCYTSEYFGLLQSINNLVLVEDNNTLTLNNDQRAKIIEYAHFKNEIKYSEIRKLL
DIEPEILFKAHNLTHKNPSGNNESKKFYEMKSYHKLKSTLPTDIWGKLHSNKESLDNLFY
CLTVYKNDNEIKDYLQANNLDYLIEYIAKLPTFNKFKHLSLVAMKRIIPFMEKGYKYSD
ACNMAELDFTGSSKLEKCNKLTVEPIIENVTNPVVIRALTQARKVINAIIQKYGLPYMVNI
ELAREAGMTRQDRDNLKKEHENNRKAREKISDLIRQNGRVASGLDILKWRLWEDQGG
RCAYSGKPIPVCDLLNDSLTQIDHIYPYSRSMDDSYMNKVLVLTDENQNKRSYTPYEVW
GSTEKWEDFEARIYSMHLPQSKEKRLLNRNFITKDLDSFISRNLNDTRYISRFLKNYIESY
LQFSNDSPKSCVVCVNGQCTAQLRSRWGLNKNREESDLHHALDAAVIACADRKIIKEIT
NYYNERENHNYKVKYPLPWHSFRQDLMETLAGVFISRAPRRKITGPAHDETIRSPKHEN
KGLTSVKIPLTTVTLEKLETMVKNTKGGISDKAVYNVLKNRLIEHNNKPLKAFAEKIYKP
LKNGTNGAIIRSIRVETPSYTGVFRNEGKGISDNSLMVRVDVFKKKDKYYLVPIYVAHMI
KKELPSKAIVPLKPESQWELIDSTHEFLFSLYQNDYLVIKTKKGITEGYYRSCHRGTGSLS
LMPHFANNKNVKIDIGVRTAISIEKYNVDILGNKSIVKGEPRRGMEKYNSFKSN
SEQ ID NO: 755 [I-TEVI WT NUCLEASE DOMAIN, LINKER DOMAIN with GGSGGS, and
Clostridium cellulolyticum Cas9]
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNGGSGGSMK
YTLGLDVGIASVGWAVIDKDNNKIIDLGVRCFDKAEESKTGESLATARRIARGMRRRISR
RSQRLRLVKKLFVQYEIIKDSSEFNRIFDTSRDGWKDPWELRYNALSRILKPYELVQVLT
HITKRRGFKSNRKEDLSTTKEGVVITSIKNNSEMLRTKNYRTIGEMIFMETPENSNKRNK
VDEYIHTIAREDLLNEIKYIFSIQRKLGSPFVTEKLEHDFLNIWEFQRPFASGDSILSKVGK
CTLLKEELRAPTSCYTSEYFGLLQSINNLVLVEDNNTLTLNNDQRAKIIEYAHFKNEIKYS
EIRKLLDIEPEILFKAHNLTHKNPSGNNESKKFYEMKSYHKLKSTLPTDIWGKLHSNKES
LDNLFYCLTVYKNDNEIKDYLQANNLDYLIEYIAKLPTFNKFKHLSLVAMKRIIPFMEKG
YKYSDACNMAELDFTGSSKLEKCNKLTVEPIIENVTNPVVIRALTQARKVINAIIQKYGL
PYMVNIELAREAGMTRQDRDNLKKEHENNRKAREKISDLIRQNGRVASGLDILKWRLW
EDQGGRCAYSGKPIPVCDLLNDSLTQIDHIYPYSRSMDDSYMNKVLVLTDENQNKRSYT
PYEVWGSTEKWEDFEARIYSMHLPQSKEKRLLNRNFITKDLDSFISRNLNDTRYISRFLK
NYIESYLQFSNDSPKSCVVCVNGQCTAQLRSRWGLNKNREESDLHHALDAAVIACADR
KIIKEITNYYNERENHNYKVKYPLPWHSFRQDLMETLAGVFISRAPRRKITGPAHDETIRS
PKHFNKGLTSVKIPLTTVTLEKLETMVKNTKGGISDKAVYNVLKNRLIEHNNKPLKAFA
EKIYKPLKNGTNGAIIRSIRVETPSYTGVFRNEGKGISDNSLMVRVDVFKKKDKYYLVPI
YVAHMIKKELPSKAIVPLKPESQWELIDSTHEFLFSLYQNDYLVIKTKKGITEGYYRSCH
RGTGSLSLMPHFANNKNVKIDIGVRTAISIEKYNVDILGNKSIVKGEPRRGMEKYNSFKS
N
SEQ ID NO: 736 [I-TEVI WT NUCLEASE DOMAIN, LINKER DOMAIN, and Geobacillus
thermodenitrificansT1 Cas9]
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNMKYKIGLDI
GITSIGWAVINLDIPRIEDLGVRIFDRAENPKTGESLALPRRLARSARRRLRRRKHRLERIR
RLFVREGILTKEELNKLFEKKHEIDVWQLRVEALDRKLNNDELARILLHLAKRRGFRSN
RKSERTNKENSTMLKHIEENQSILSSYRTVAEMVVKDPKFSLHKRNKEDNYTNTVARD
DLEREIKLIFAKQREYGNIVCTEAFEHEYISIWASQRPFASKDDIEKKVGFCTFEPKEKRA
PKATYTFQSFTVWEHINKLRLVSPGGIRALTDDERRLIYKQAFHKNKITFHDVRTLLNLP
DDTRFKGLLYDRNTTLKENEKVRFLELGAYHKIRKAIDSVYGKGAAKSFRPIDFDTFGY
ALTMFKDDTDIRSYLRNEYEQNGKRMENLADKVYDEELIEELLNLSFSKFGHLSLKALR
NILPYMEQGEVYSTACERAGYTFTGPKKKQKTVLLPNIPPIANPVVMRALTQARKVVNA
IIKKYGSPVSIHIELARELSQSFDERRKMQKEQEGNRKKNETAIRQLVEYGLTLNPTGLDI
VKFKLWSEQNGKCAYSLQPIEIERLLEPGYTEVDHVIPYSRSLDDSYTNKVLVLTKENRE
KGNRTPAEYLGLGSERWQQFETFVLTNKQFSKKKRDRLLRLHYDENEENEFKNRNLND
TRYISRFLANFIREHLKFADSDDKQKVYTVNGRITAHLRSRWNFNKNREESNLHHAVDA
AIVACTTPSDIARVTAFYQRREQNKELSKKTDPQFPQPWPHFADELQARLSKNPKESIKA
LNLGNYDNEKLESLQPVFVSRMPKRSITGAAHQETLRRYIGIDERSGKIQTVVKKKLSEI
QLDKTGHFPMYGKESDPRTYEAIRQRLLEHNNDPKKAFQEPLYKPKKNGELGPIIRTIKII
DTTNQVIPLNDGKTVAYNSNIVRVDVFEKDGKYYCVPIYTIDMMKGILPNKAIEPNKPYS
EWKEMTEDYTFRFSLYPNDLIRIEFPREKTIKTAVGEEIKIKDLFAYYQTIDSSNGGLSLVS
HDNNFSLRSIGSRTLKRFEKYQVDVLGNIYKVRGEKRVGVASSSHSKAGETIRPL
SEQ ID NO: 756 [I-TEVI WT NUCLEASE DOMAIN, LINKER DOMAIN with GGSGGS, and
Geobacillus thermodenitrificansT1 Cas9]
MGKSGIYQIKNTLNNKVYVGSAKDFEKRWKRHFKDLEKGCHSSIKLQRSFNKHGNVFE
CSILEEIPYEKDLIIERENFWIKELNSKINGYNIADATFGDTCSTHPLKEEIIKKRSETVKAK
MLKLGPDGRKALYSKPGSKNGRWNPETHKFCKCGVRIQTSAYTCSKCRNGGSGGSMK
YKIGLDIGITSIGWAVINLDIPRIEDLGVRIFDRAENPKTGESLALPRRLARSARRRLRRRK
HRLERIRRLFVREGILTKEELNKLFEKKHEIDVWQLRVEALDRKLNNDELARILLHLAKR
RGFRSNRKSERTNKENSTMLKHIEENQSILSSYRTVAEMVVKDPKFSLHKRNKEDNYTN
TVARDDLEREIKLIFAKQREYGNIVCTEAFEHEYISIWASQRPFASKDDIEKKVGFCTFEP
KEKRAPKATYTFQSFTVWEHINKLRLVSPGGIRALTDDERRLIYKQAFHKNKITFHDVRT
LLNLPDDTRFKGLLYDRNTTLKENEKVRFLELGAYHKIRKAIDSVYGKGAAKSFRPIDFD
TFGYALTMFKDDTDIRSYLRNEYEQNGKRMENLADKVYDEELIEELLNLSFSKFGHLSL
KALRNILPYMEQGEVYSTACERAGYTFTGPKKKQKTVLLPNIPPIANPVVMRALTQARK
VVNAIIKKYGSPVSIHIELARELSQSFDERRKMQKEQEGNRKKNETAIRQLVEYGLTLNP
TGLDIVKFKLWSEQNGKCAYSLQPIEIERLLEPGYTEVDHVIPYSRSLDDSYTNKVLVLT
KENREKGNRTPAEYLGLGSERWQQFETFVLTNKQFSKKKRDRLLRLHYDENEENEFKN
RNLNDTRYISRFLANFIREHLKFADSDDKQKVYTVNGRITAHLRSRWNFNKNREESNLH
HAVDAAIVACTTPSDIARVTAFYQRREQNKELSKKTDPQFPQPWPHFADELQARLSKNP
KESIKALNLGNYDNEKLESLQPVFVSRMPKRSITGAAHQETLRRYIGIDERSGKIQTVVK
KKLSEIQLDKTGHFPMYGKESDPRTYEAIRQRLLEHNNDPKKAFQEPLYKPKKNGELGPI
IRTIKIIDTTNQVIPLNDGKTVAYNSNIVRVDVFEKDGKYYCVPIYTIDMMKGILPNKAIE
PNKPYSEWKEMTEDYTFRFSLYPNDLIRIEFPREKTIKTAVGEEIKIKDLFAYYQTIDSSN
GGLSLVSHDNNFSLRSIGSRTLKRFEKYQVDVLGNIYKVRGEKRVGVASSSHSKAGETIR
PL
SEQ ID NO: 740 [SV40 nuclear localization sequence]
PKKKRKV
SEQ ID NO: 741 [nucleoplasmin nuclear localization sequence]
KRPAATKKAGQAKKKK

1-146. (canceled)

147. A composition, comprising: a chimeric nuclease, wherein the chimeric nuclease comprises:
(a) an I-TEVI nuclease domain, wherein the I-TEVI nuclease domain comprises a mutation at any one of positions corresponding to T11, V16, N14, E25, K26, R27, E36, K37, G38, C39, S41, L45, F49, I60, and E81 of SEQ ID NO: 700, or a combination thereof,
(b) an RNA-guided nuclease Cas domain; and
(c) a guide RNA, wherein the guide RNA comprises a nucleic acid sequence that targets an oncogenic mutation, wherein the oncogenic mutation is
(i) an insertion of one or more nucleotides;
(ii) a substitution or deletion of 10 or less nucleotides; or
(iii) a single nucleotide polymorphism.

148. The composition of claim 147, wherein the I-TEVI nuclease domain comprises a mutation selected from a mutation corresponding to any one of T11V, V161, N14G, E25D, K26R, R27A, E36S, K37N, G38N, C39V, S41H, L45F, F49Y, I60V, E811, or a combination thereof.

149. The composition of claim 147, wherein the oncogenic mutation is an oncogenic mutation to a gene selected from any one of EGFR, Muc4, PIK3CA, KRAS, or a combination thereof.

150. The composition of claim 147, wherein the oncogenic mutation is not a deletion in exon 19 of EGFR.

151. The composition of claim 147, wherein a sequence comprising the oncogenic mutation is selected from a mutation set forth in any one of SEQ ID NOs: 1-683.

152. The composition of claim 147, wherein the oncogenic mutation comprises a mutation corresponding to an EGFR L858R mutation or an EGFR V769_D770insASV mutation.

153. The composition of claim 147, wherein the guide RNA hybridizes to a target nucleotide sequence set forth in SEQ ID NOs: 45, 130, or 141, or comprises a nucleotide sequence as set forth in SEQ ID NOs: 1045, 1130, 1141, or 1686.

154. The composition of claim 147, wherein the guide RNA hybridizes to a target nucleotide sequence set forth in SEQ ID NO: 683, or comprises a nucleotide sequence as set forth in SEQ ID NOs: 1683 or 1684.

155. The composition of claim 147, further comprising a linker that is operably linked to the I-TEVI nuclease domain and the RNA-guided nuclease Cas domain.

156. The composition of claim 147, wherein the RNA-guided nuclease Cas domain is an RNA-guided nuclease Cas9 domain.

157. The composition of claim 156, wherein the RNA-guided nuclease Cas9 domain is any one of an RNA-guided nuclease Staphylococcus aureus Cas9 domain, an RNA-guided nuclease Streptococcus pyogenes Cas9 domain, an RNA-guided nuclease Neisseria meningitidis Cas9 domain, an RNA-guided nuclease Campylobacter jejuni Cas9 domain, an RNA-guided nuclease Streptococcus pasteurianus Cas9 domain, an RNA-guided nuclease Streptococcus pasteurianus Cas9 domain, an RNA-guided nuclease Clostridium cellulolyticum Cas9 domain, or an RNA-guided nuclease Geobacillus thermodenitrificans T1 Cas9 domain.

158. The composition of claim 157, wherein the RNA-guided nuclease Staphylococcus aureus Cas9 domain comprises a mutation corresponding to the D10E mutation.

159. The composition of claim 147, wherein the I-TEVI nuclease domain comprises an amino acid sequence that is at least 85%, 90%, 95%, 97%, 98%, or 99% identical to SEQ ID NO: 700.

160. The composition of claim 147, wherein the composition further comprises a donor nucleic acid.

161. The composition of claim 147, wherein the donor nucleic acid restores a non-oncogenic function of a gene comprising the oncogenic mutation.

162. A nucleic acid or plurality of nucleic acids encoding the chimeric nuclease or the guide RNA of claim 147, optionally further comprising a donor nucleic acid portion.

163. The nucleic acid or plurality of nucleic acids of claim 162, wherein the nucleic acid is an expression vector selected from a plasmid, a lentivirus vector, an adeno associated virus vector, or an adenovirus vector.

164. A method of silencing or disrupting at least a portion of the oncogenic mutation in a cell comprising contacting the composition of claim 147 to the cell.

165. A method of replacing at least a portion of the oncogenic mutation in a cell comprising contacting the composition of claim 160 to the cell.

166. A method of treating cancer in an individual comprising administering the composition of claim 147 to the individual with cancer, thereby treating the cancer in the individual.


SEQUENCE LISTING (1690 sequences)

SEQ ID NO: 1 - DNA, 49 bp, Homo sapiens
  source 1..49: mol_type = genomic DNA
ctgtggacattggagagttgacccaaacaaaagtgatctcctccagaag

SEQ ID NO: 2 - DNA, 50 bp, Homo sapiens
  source 1..50: mol_type = genomic DNA
caaagttgtcttgtttcatcaaaaaattcttccctttctgcttcttgagt

SEQ ID NO: 3 - DNA, 50 bp, Homo sapiens
  source 1..50: mol_type = genomic DNA
cgatgctgaggaggggccagacctaagagcaatcagtgaggaatcagagg

SEQ ID NO: 4 - DNA, 50 bp, Homo sapiens
  source 1..50: mol_type = genomic DNA
cagcggtggcccacagcagctgctggcccagggacagctcagtgcagggt

SEQ ID NO: 5 - DNA, 49 bp, Homo sapiens
  source 1..49: mol_type = genomic DNA
caaagcaatttctacacgagatcctctctctaaaatcactgagcaggag

SEQ ID NO: 6 - DNA, 49 bp, Homo sapiens
  source 1..49: mol_type = genomic DNA
caaagcaatttctacacgagatcctctctctgaaatcactgagcgggag

SEQ ID NO: 7 - DNA, 49 bp, Homo sapiens
  source 1..49: mol_type = genomic DNA
caaagcaatttctacacgagatcctctctctgaaatcactaagcaggag

SEQ ID NO: 8 - DNA, 49 bp, Homo sapiens
  source 1..49: mol_type = genomic DNA
caaagcaatttctacacgagatcctctctctgaaatcactgcgcaggag

SEQ ID NO: 9 - DNA, 49 bp, Homo sapiens
  source 1..49: mol_type = genomic DNA
catggtttaataaaaaaaaaaaaaaataggcgtctcaggcagatggagg

SEQ ID NO: 10 - DNA, 48 bp, Homo sapiens
  source 1..48: mol_type = genomic DNA
cgtcggtgacaggaagagaggtggcgtgacctgtggatactgaggaag

SEQ ID NO: 11 - DNA, 48 bp, Homo sapiens
  source 1..48: mol_type = genomic DNA
cgtcggtgacaggaagagaggtggcgtgacctgtgggcactgaggaag

SEQ ID NO: 12 - DNA, 49 bp, Homo sapiens
  source 1..49: mol_type = genomic DNA
cttggcaaactcccccagcttggaggctgcggcccaccgcaccatgggg

SEQ ID NO: 13 - DNA, 48 bp, Homo sapiens
  source 1..48: mol_type = genomic DNA
catggtttaataaaaaaaaaaaaaaataggcgtctcaggcagatggag

SEQ ID NO: 14 - DNA, 49 bp, Homo sapiens
  source 1..49: mol_type = genomic DNA
ctcgggtaagatgctgaggaggggccagacctaagagcaatcagtgagg

SEQ ID NO: 15 - DNA, 49 bp, Homo sapiens
  source 1..49: mol_type = genomic DNA
ccagggagcactaagtgaggtaagcaagcaggacaagaagcggtggagg

SEQ ID NO: 16 - DNA, 50 bp, Homo sapiens
  source 1..50: mol_type = genomic DNA
cttggcaaactcccccagcttggaggctgcggcccaccgcaccatggggg

SEQ ID NO: 17 - DNA, 50 bp, Homo sapiens
  source 1..50: mol_type = genomic DNA
caccgaagagctaagcgacttctgaggagaccggaaaatgggaggcgggg

SEQ ID NO: 18 - DNA, 50 bp, Homo sapiens
  source 1..50: mol_type = genomic DNA
cctgggtccctcgcaccatgcggaggttgggcaatggtagagtagagaat

SEQ ID NO: 19 - DNA, 50 bp, Homo sapiens
  source 1..50: mol_type = genomic DNA
catcgctctggtggagagagggaagaaaggaggagtggggatgggagaat

SEQ ID NO: 20 - DNA, 48 bp, Homo sapiens
  source 1..48: mol_type = genomic DNA
ctgagctgtccctgggccagcagctgctgtgggccaccgctgatgagg

SEQ ID NO: 21 - DNA, 48 bp, Homo sapiens
  source 1..48: mol_type = genomic DNA
caccgttttccttttagctttacttacagtgtctgaagaagtttgaag

SEQ ID NO: 22 - DNA, 47 bp, Homo sapiens
  source 1..47: mol_type = genomic DNA
catcgctctggtggagagagggaagaaaggaggagtggggatgggag

SEQ ID NO: 23 - DNA, 50 bp, Homo sapiens
  source 1..50: mol_type = genomic DNA
catgggcgtcatgaaccggaggcccatcctcaccatcatcacactggaag

SEQ ID NO: 24 - DNA, 49 bp, Homo sapiens
  source 1..49: mol_type = genomic DNA
catgggcaagtgaaatgattactagtcaaatccgtcagtgatcccgagt

SEQ ID NO: 25 - DNA, 48 bp, Homo sapiens
  source 1..48: mol_type = genomic DNA
ctgggctgttcccgcccctatgcccttttttgggttttcggccagagg

SEQ ID NO: 26 - DNA, 49 bp, Homo sapiens
  source 1..49: mol_type = genomic DNA
catggtttaaataaaaaaaaaaaaaataggcgtctcaggcagatggagg

SEQ ID NO: 27 - DNA, 50 bp, Homo sapiens
  source 1..50: mol_type = genomic DNA
catgggcgacatgaaccggaggcccatcctcaccatcatcacactggaag

SEQ ID NO: 28 - DNA, 48 bp, Homo sapiens
  source 1..48: mol_type = genomic DNA
ccagggagcactaagtgaggtaagcaagcaggacaagaagcggtggag

SEQ ID NO: 29 - DNA, 47 bp, Homo sapiens
  source 1..47: mol_type = genomic DNA
cactggcatgccgatagcaaaatctttaataaaattacataaagaat

SEQ ID NO: 30 - DNA, 46 bp, Homo sapiens
  source 1..46: mol_type = genomic DNA
ctctggaatccattctggtgccactgccacagctccttctctgagt

SEQ ID NO: 31 - DNA, 46 bp, Homo sapiens
  source 1..46: mol_type = genomic DNA
ctctggaatccattttggtgccactaccacagctccttctctgagt

SEQ ID NO: 32 - DNA, 48 bp, Homo sapiens
  source 1..48: mol_type = genomic DNA
catggtttaaataaaaaaaaaaaaaataggcgtctcaggcagatggag

SEQ ID NO: 33 - DNA, 49 bp, Homo sapiens
  source 1..49: mol_type = genomic DNA
cgcaggagaaagattttctatggagtcacaggtaagtgctaaaatggag

SEQ ID NO: 34 - DNA, 46 bp, Homo sapiens
  source 1..46: mol_type = genomic DNA
ctctggaatccattctggtgccactaccacagctcctcctctgagt

SEQ ID NO: 35 - DNA, 50 bp, Homo sapiens
  source 1..50: mol_type = genomic DNA
cagggtaggtcttggccagttggcaaaacatcttgttgagggcaggggag

SEQ ID NO: 36 - DNA, 48 bp, Homo sapiens
  source 1..48: mol_type = genomic DNA
cgcgggtgccgggcgggggtgtggaatcaacccacagctgcacagggt

SEQ ID NO: 37 - DNA, 49 bp, Homo sapiens
  source 1..49: mol_type = genomic DNA
cagtgttacttacctgtcttgtctttgttgatgtttcaataaaaggaat

SEQ ID NO: 38 - DNA, 50 bp, Homo sapiens
  source 1..50: mol_type = genomic DNA
ccatgggcaagtgaaatgattactagtcaaatccgtcagtgatcccgagt

SEQ ID NO: 39 - DNA, 49 bp, Homo sapiens
  source 1..49: mol_type = genomic DNA
ctcagataagatgctgaggaggggccagacctaagagcaatcagtgagg

SEQ ID NO: 40 - DNA, 48 bp, Homo sapiens
  source 1..48: mol_type = genomic DNA
cagggtaggtcttggccagttggcaaaacatcttgttgagggcagggg

SEQ ID NO: 41 - DNA, 49 bp, Homo sapiens
  source 1..49: mol_type = genomic DNA
cacaggtttccaccgtgcacattatgaagaacagaaatggaggtgggag

SEQ ID NO: 42 - DNA, 49 bp, Homo sapiens
  source 1..49: mol_type = genomic DNA
cttggatattctcgacacagcaggtcgagaggagtacagtgcaatgagg

SEQ ID NO: 43 - DNA, 48 bp, Homo sapiens
  source 1..48: mol_type = genomic DNA
caccgcttcttgtcctgcttgcttacctcacttagtgctccctggggg

SEQ ID NO: 44 - DNA, 50 bp, Homo sapiens
  source 1..50: mol_type = genomic DNA
catgggcggcatgaaccggagtcccatcctcaccatcatcacactggaag

SEQ ID NO: 45 - DNA, 50 bp, Homo sapiens
  source 1..50: mol_type = genomic DNA
caccgcagcatgtcaagatcacagattttgggcgggccaaactgctgggt

SEQ ID NO: 46 - DNA, 47 bp, Homo sapiens
  source 1..47: mol_type = genomic DNA
cgagggtgcagaatcccacgtccgtagaaaggtagttgtctaaggag

SEQ ID NO: 47 - DNA, 47 bp, Homo sapiens
  source 1..47: mol_type = genomic DNA
ctctgcttctttacatctcagttagtcccttctgaaattcagtgagg

SEQ ID NO: 48 - DNA, 47 bp, Homo sapiens
  source 1..47: mol_type = genomic DNA
ctgaggaaaggctggtgacatgaagaggggtggcgtgacctgtggat

SEQ ID NO: 49 - DNA, 50 bp, Homo sapiens
  source 1..50: mol_type = genomic DNA
catgggcggcatgaaccagaggcccatcctcaccatcatcacactggaag

SEQ ID NO: 50 - DNA, 50 bp, Homo sapiens
  source 1..50: mol_type = genomic DNA
catgggcggcatgaactggaggcccatcctcaccatcatcacactggaag

SEQ ID NO: 51 - DNA, 49 bp, Homo sapiens
  source 1..49: mol_type = genomic DNA
cttggatattctcgacacagcaggtctagaggagtacagtgcaatgagg

SEQ ID NO: 52 - DNA, 49 bp, Homo sapiens
  source 1..49: mol_type = genomic DNA
cttggatattctcgacacagcaggtcacgaggagtacagtgcaatgagg

SEQ ID NO: 53 - DNA, 50 bp, Homo sapiens
  source 1..50: mol_type = genomic DNA
cacaggtttccaccgtgcacattatgaagaacagaaatggaggtgggagt

SEQ ID NO: 54 - DNA, 48 bp, Homo sapiens
  source 1..48: mol_type = genomic DNA
caaagtacatgaacttgtcttcccgtcatgtgggtcctgaattggagg

SEQ ID NO: 55 - DNA, 46 bp, Homo sapiens
  source 1..46: mol_type = genomic DNA
cctagcactgcccaacaacaccagctcctctccctagccaaagaag

SEQ ID NO: 56 - DNA, 47 bp, Homo sapiens
  source 1..47: mol_type = genomic DNA
ctcagggcaactgacagtgcaagtcacagacttggctgtcccagaat

SEQ ID NO: 57 - DNA, 47 bp, Homo sapiens
  source 1..47: mol_type = genomic DNA
caccgcttcttgtcctgcttgcttacctcacttagtgctccctgggg

SEQ ID NO: 58 - DNA, 49 bp, Homo sapiens
  source 1..49: mol_type = genomic DNA
cctgggagagaccggcgcacaaaggaagagaatctccgcaagaaagggg

SEQ ID NO: 59 - DNA, 47 bp, Homo sapiens
  source 1..47: mol_type = genomic DNA
cactgattgctcttaggtctggcccctcctcagcgtcttatccgagt

SEQ ID NO: 60 - DNA, 47 bp, Homo sapiens
  source 1..47: mol_type = genomic DNA
cactgattgctcttaggtctggcccctcctcagcatcgtatccgagt

SEQ ID NO: 61 - DNA, 49 bp, Homo sapiens
  source 1..49: mol_type = genomic DNA
cctgggagagactggcgcacagaggaagagaatctccgcaagaaagggg

SEQ ID NO: 62 - DNA, 49 bp, Homo sapiens
  source 1..49: mol_type = genomic DNA
cacagcaggtcgagaggagtacagtgcaatgagggaccagtacatgagg

SEQ ID NO: 63 - DNA, 49 bp, Homo sapiens
  source 1..49: mol_type = genomic DNA
cacagcaggtcacgaggagtacagtgcaatgagggaccagtacatgagg

SEQ ID NO: 64 - DNA, 48 bp, Homo sapiens
  source 1..48: mol_type = genomic DNA
cccagctgctcaccatcgctatctgagcagcgctcatggtgggggaag

SEQ ID NO: 65 - DNA, 50 bp, Homo sapiens
  source 1..50: mol_type = genomic DNA
ctgggaaagaccggcgcacagaggaagagaatctccgcaagaaaggggag

SEQ ID NO: 66 - DNA, 48 bp, Homo sapiens
  source 1..48: mol_type = genomic DNA
ctgggaaagaccggcgcacagaggaagagaatctccgcaagaaagggg

SEQ ID NO: 67 - DNA, 47 bp, Homo sapiens
  source 1..47: mol_type = genomic DNA
ctgaggaaaagctggtgacaggaagaggggtggcctgacctgtggat

SEQ ID NO: 68 - DNA, 50 bp, Homo sapiens
  source 1..50: mol_type = genomic DNA
ctcagtttcctcactgaatttcagaagggactaactgagatgtaaagaag

SEQ ID NO: 69 - DNA, 47 bp, Homo sapiens
  source 1..47: mol_type = genomic DNA
cactgattgctcttaggtctggcccctcctcagcatcttacccgagt

SEQ ID NO: 70 - DNA, 49 bp, Homo sapiens
  source 1..49: mol_type = genomic DNA
cccagggaagtggttgacccctccggtggctgggccactctgctagagt

SEQ ID NO: 71 - DNA, 47 bp, Homo sapiens
  source 1..47: mol_type = genomic DNA
cactgattgctcttaggtctggcccctccttagcatcttatccgagt

SEQ ID NO: 72 - DNA, 48 bp, Homo sapiens
  source 1..48: mol_type = genomic DNA
ctgggacagaccggcgcacagaggaagagaatctccgcaagaaagggg

SEQ ID NO: 73 - DNA, 46 bp, Homo sapiens
  source 1..46: mol_type = genomic DNA
cacagatgatggaattcttgcttgtgagctttactgagaattgggt

SEQ ID NO: 74 - DNA, 49 bp, Homo sapiens
  source 1..49: mol_type = genomic DNA
cctgggagagaccggcgcacagagaaagagaatctccgcaagaaagggg

SEQ ID NO: 75 - DNA, 50 bp, Homo sapiens
  source 1..50: mol_type = genomic DNA
ctgggacagaccggcgcacagaggaagagaatctccgcaagaaaggggag

SEQ ID NO: 76 - DNA, 48 bp, Homo sapiens
  source 1..48: mol_type = genomic DNA
ccagggaagtggttgacccctccggtggctgggccactctgctagagt

SEQ ID NO: 77 - DNA, 49 bp, Homo sapiens
  source 1..49: mol_type = genomic DNA
cacagcaggtctagaggagtacagtgcaatgagggaccagtacatgagg

SEQ ID NO: 78 - DNA, 50 bp, Homo sapiens
  source 1..50: mol_type = genomic DNA
catgggcagcatgaaccggaggcccatcctcaccatcatcacactggaag

SEQ ID NO: 79 - DNA, 49 bp, Homo sapiens
  source 1..49: mol_type = genomic DNA
ctcggataagatgctaaggaggggccagacctaagagcaatcagtgagg

SEQ ID NO: 80 - DNA, 47 bp, Homo sapiens
  source 1..47: mol_type = genomic DNA
caaagtacatgaacttgtcttcccgtcatgtgggtcctgaattggag

SEQ ID NO: 81 - DNA, 49 bp, Homo sapiens
  source 1..49: mol_type = genomic DNA
cctgggacagaccggcgcacagaggaagagaatctccgcaagaaagggg

SEQ ID NO: 82 - DNA, 50 bp, Homo sapiens
  source 1..50: mol_type = genomic DNA
ctgtgactgcttgtagatggccatggcgtggacgcgggtgccgggcgggg

SEQ ID NO: 83 - DNA, 49 bp, Homo sapiens
  source 1..49: mol_type = genomic DNA
ctcggataagacgctgaggaggggccagacctaagagcaatcagtgagg

SEQ ID NO: 84 - DNA, 47 bp, Homo sapiens
  source 1..47: mol_type = genomic DNA
ctgaggaacggctggtgacaggaagagaggtggcgtggcctgtggat

SEQ ID NO: 85 - DNA, 50 bp, Homo sapiens
  source 1..50: mol_type = genomic DNA
ctgtgactgcttgtagatggccatggcgaggacgcgggtgccgggcgggg

SEQ ID NO: 86 - DNA, 46 bp, Homo sapiens
  source 1..46: mol_type = genomic DNA
cagagttgttagcggttctcaagatgccactcttagggtttgggat

SEQ ID NO: 87 - DNA, 50 bp, Homo sapiens
  source 1..50: mol_type = genomic DNA
ctgtgactgcttgtagatggccatggcgcggaagcgggtgccgggcgggg

SEQ ID NO: 88 - DNA, 50 bp, Homo sapiens
  source 1..50: mol_type = genomic DNA
ctgcggcccgccgcaccatgcgggtgtcatctgagcacaggttccggaag

SEQ ID NO: 89 - DNA, 49 bp, Homo sapiens
  source 1..49: mol_type = genomic DNA
cctgggaaagaccggcgcacagaggaagagaatctccgcaagaaagggg

SEQ ID NO: 90 - DNA, 46 bp, Homo sapiens
  source 1..46: mol_type = genomic DNA
ccgagcaggctccggcatcctccggacccaccatctaggggtgggt

SEQ ID NO: 91 - DNA, 46 bp, Homo sapiens
  source 1..46: mol_type = genomic DNA
ccgagcaggctccggcatcctccggacccaccatttgggggtgggt

SEQ ID NO: 92 - DNA, 47 bp, Homo sapiens
  source 1..47: mol_type = genomic DNA
ctgaggaagcgtcggtgacaggaagagaggtggcgtgacctgtggat

SEQ ID NO: 93 - DNA, 47 bp, Homo sapiens
  source 1..47: mol_type = genomic DNA
cactggcttctcatttgcattaaactgtgaactcctttagggtgggg

SEQ ID NO: 94 - DNA, 50 bp, Homo sapiens
  source 1..50: mol_type = genomic DNA
ccttggcaaactcccccagcttggaggctgcggcccaccgcaccatgggg

SEQ ID NO: 95 - DNA, 49 bp, Homo sapiens
  source 1..49: mol_type = genomic DNA
ctcagaacatctcgaagcgctcacgcccacggatctgcagcaacagagg

SEQ ID NO: 96 - DNA, 46 bp, Homo sapiens
  source 1..46: mol_type = genomic DNA
catggccatctgcaagcagtcacagcacatgacggaggttgtgagg

SEQ ID NO: 97 - DNA, 50 bp, Homo sapiens
  source 1..50: mol_type = genomic DNA
ctcagcatcttacccgagtggaaggaaatttgcgtgtggagtatttggat

SEQ ID NO: 98 - DNA, 46 bp, Homo sapiens
  source 1..46: mol_type = genomic DNA
ctgagggaactcaaagtacatgaacttgtcttcccgtcatgtgggt

SEQ ID NO: 99 - DNA, 49 bp, Homo sapiens
  source 1..49: mol_type = genomic DNA
cttggaggtcctgcccctgggacttgtccggctcatacggagtgaggag

SEQ ID NO: 100 - DNA, 46 bp, Homo sapiens
  source 1..46: mol_type = genomic DNA
cccagctgctcaccatcgctatctgagcagcgctcatggtgggggt

SEQ ID NO: 101 - DNA, 50 bp, Homo sapiens
  source 1..50: mol_type = genomic DNA
ctgcggcccaccgcaccatgggggtgtcatctgagcacaggttccggaag

SEQ ID NO: 102 - DNA, 47 bp, Homo sapiens
  source 1..47: mol_type = genomic DNA
ccatggccatctgcaagcagtcacagcacatgacggaggttgtgagg

SEQ ID NO: 103 - DNA, 49 bp, Homo sapiens
  source 1..49: mol_type = genomic DNA
ctcagatgacacccccatggtgcggtgggccgcagcctccaagctgggg

SEQ ID NO: 104 - DNA, 47 bp, Homo sapiens
  source 1..47: mol_type = genomic DNA
ctgaggaagggatggtgacaggaagaggggtggcgtgaccggtggat

SEQ ID NO: 105 - DNA, 46 bp, Homo sapiens
  source 1..46: mol_type = genomic DNA
cacagctcctcctctgagtggtaaaggcaatcctgaggaagaggat

SEQ ID NO: 106 - DNA, 50 bp, Homo sapiens
  source 1..50: mol_type = genomic DNA
ctcagcatcgtatccgagtggaaggaaatttgcgtgtggagtatttggat

SEQ ID NO: 107 - DNA, 50 bp, Homo sapiens
  source 1..50: mol_type = genomic DNA
ctcagcatcttatccgagtggaaggaaatttgcgtgtggagtgtttggat

SEQ ID NO: 108 - DNA, 49 bp, Homo sapiens
  source 1..49: mol_type = genomic DNA
ctcggatacgatgctgaggaggggccagacctaagagcaatcagtgagg

SEQ ID NO: 109 - DNA, 50 bp, Homo sapiens
  source 1..50: mol_type = genomic DNA
ctttgcctcctggggcggccgccacccacccccaaatggtgggtccggag

SEQ ID NO: 110 - DNA, 50 bp, Homo sapiens
  source 1..50: mol_type = genomic DNA
ctcagcgtcttatccgagtggaaggaaatttgcgtgtggagtatttggat

SEQ ID NO: 111 - DNA, 50 bp, Homo sapiens
  source 1..50: mol_type = genomic DNA
ctttgcctcctggggcggccgccacccacccctagatggtgggtccggag

SEQ ID NO: 112 - DNA, 48 bp, Homo sapiens
  source 1..48: mol_type = genomic DNA
ctgtggcgggggcgtctctgcaggccagggtcctgggcgctcgtgaag

SEQ ID NO: 113 - DNA, 50 bp, Homo sapiens
  source 1..50: mol_type = genomic DNA
ctcagcatcttatctgagtggaaggaaatttgcgtgtggagtatttggat

SEQ ID NO: 114 - DNA, 47 bp, Homo sapiens
  source 1..47: mol_type = genomic DNA
ctgggcgctcgtgaagatggagccatattcctgcaggcgctctggag

SEQ ID NO: 115 - DNA, 49 bp, Homo sapiens
  source 1..49: mol_type = genomic DNA
ccgcgtcctcgccatggccatctacaagcagtcacagcacatgacggag

SEQ ID NO: 116 - DNA, 50 bp, Homo sapiens
  source 1..50: mol_type = genomic DNA
ctgtgtcctttcaggatggtggatgtggggggcctgcggtcggagcggag

SEQ ID NO: 117 - DNA, 49 bp, Homo sapiens
  source 1..49: mol_type = genomic DNA
ctcagatgacacccgcatggtgcggcgggccgcagcctccaagctgggg

SEQ ID NO: 118 - DNA, 50 bp, Homo sapiens
  source 1..50: mol_type = genomic DNA
ctcagatgacacccccatggtgcggtgggccgcagcctccaagctggggg

SEQ ID NO: 119 - DNA, 50 bp, Homo sapiens
  source 1..50: mol_type = genomic DNA
cattgcccaacctccgcatggtgcgagggacccaggtctacgatgggaag

SEQ ID NO: 120 - DNA, 49 bp, Homo sapiens
  source 1..49: mol_type = genomic DNA
ccgcgtccacgccatggccatctacaagcagtcacagcacatgacggag

SEQ ID NO: 121 - DNA, 47 bp, Homo sapiens
  source 1..47: mol_type = genomic DNA
ctgaggaagtgccggtgacaggaagagcggtggcctgacctgtggat

SEQ ID NO: 122 - DNA, 50 bp, Homo sapiens
  source 1..50: mol_type = genomic DNA
ctgtgactgcttgcagatggccatggcgcggacgcgggtgccgggcgggg

SEQ ID NO: 123 - DNA, 47 bp, Homo sapiens
  source 1..47: mol_type = genomic DNA
cagggaagtggttgacccctccggtggctgggccactctgctagagt

SEQ ID NO: 124 - DNA, 46 bp, Homo sapiens
  source 1..46: mol_type = genomic DNA
cagagaagcgggcgcgcgggggcgggcgcgtggggccttgccggag

SEQ ID NO: 125 - DNA, 49 bp, Homo sapiens
  source 1..49: mol_type = genomic DNA
cactgaggaagcgtcggtgacaggaagagaggtggcgtgacctgtggat

SEQ ID NO: 126 - DNA, 46 bp, Homo sapiens
  source 1..46: mol_type = genomic DNA
ccaggaagccctcccgggcagcgtcgtgcacgggtcaggtgagagt

SEQ ID NO: 127 - DNA, 50 bp, Homo sapiens
  source 1..50: mol_type = genomic DNA
ccgcgtcctcgccatggccatctacaagcagtcacagcacatgacggagg

SEQ ID NO: 128 - DNA, 47 bp, Homo sapiens
  source 1..47: mol_type = genomic DNA
cttggaggtcctgcccctgggacttgtccggctcatacggagtgagg

SEQ ID NO: 129 - DNA, 50 bp, Homo sapiens
  source 1..50: mol_type = genomic DNA
ccgcgtccacgccatggccatctacaagcagtcacagcacatgacggagg

SEQ ID NO: 130 - DNA, 47 bp, Homo sapiens
  source 1..47: mol_type = genomic DNA
cacagattttgggcgggccaaactgctgggtgcggaagagaaagaat

SEQ ID NO: 131 - DNA, 47 bp, Homo sapiens
  source 1..47: mol_type = genomic DNA
cggtgggtattggggttggggtcaccgtagtggtggtggtgatgggt

SEQ ID NO: 132 - DNA, 50 bp, Homo sapiens
  source 1..50: mol_type = genomic DNA
ctcagatgacacccgcatggtgcggcgggccgcagcctccaagctggggg

SEQ ID NO: 133 - DNA, 50 bp, Homo sapiens
  source 1..50: mol_type = genomic DNA
cttggaggtcctgcccctgggacttgtccggctcatacggagtgaggagg

SEQ ID NO: 134 - DNA, 48 bp, Homo sapiens
  source 1..48: mol_type = genomic DNA
cggtgggccgcagcctccaagctgggggagtttgccaaggtgctggag

SEQ ID NO: 135 - DNA, 48 bp, Homo sapiens
  source 1..48: mol_type = genomic DNA
ctgagccaccctacagccagaagagatatgaggaaatcgttaaggaag

SEQ ID NO: 136 - DNA, 48 bp, Homo sapiens
  source 1..48: mol_type = genomic DNA
ctgggtctcacagtccacacagtgggcattcccacgcatgttttggat

SEQ ID NO: 137 - DNA, 47 bp, Homo sapiens
  source 1..47: mol_type = genomic DNA
cggtggcccacagcagctgctggcccagggacagctcagtgcagggt

SEQ ID NO: 138 - DNA, 49 bp, Homo sapiens
  source 1..49: mol_type = genomic DNA
ctgggtccctcgcaccatgcggaggttgggcaatggtagagtagagaat

SEQ ID NO: 139 - DNA, 46 bp, Homo sapiens
  source 1..46: mol_type = genomic DNA
ctccgtcatgtgctgtgactgcttgtagatggccatggcgcggaag

SEQ ID NO: 140 - DNA, 48 bp, Homo sapiens
  source 1..48: mol_type = genomic DNA
cgggggcgtctctgcaggccagggtcctgggcgctcgtgaagatggag

SEQ ID NO: 141 - DNA, 47 bp, Homo sapiens
  source 1..47: mol_type = genomic DNA
cgcagcatgtcaagatcacagattttgggcgggccaaactgctgggt

SEQ ID NO: 142 - DNA, 49 bp, Homo sapiens
  source 1..49: mol_type = genomic DNA
ccgcgtccgcgccatggccatctgcaagcagtcacagcacatgacggag

SEQ ID NO: 143 - DNA, 50 bp, Homo sapiens
  source 1..50: mol_type = genomic DNA
cgctgggctgttcccgcccctatgcccttttttgggttttcggccagagg

SEQ ID NO: 144 - DNA, 50 bp, Homo sapiens
  source 1..50: mol_type = genomic DNA
ctttggctagggagaggagctggtgttgttgggcagtgctaggaaagagg

SEQ ID NO: 145 - DNA, 49 bp, Homo sapiens
  source 1..49: mol_type = genomic DNA
cagcgggtgtcagcttgcctgacccccatgtcgcctctgtaggtagaag

SEQ ID NO: 146 - DNA, 49 bp, Homo sapiens
  source 1..49: mol_type = genomic DNA
ccgcgcggagggagaggaaatgagtgcggcggccttgccggcgtcggag

SEQ ID NO: 147 - DNA, 48 bp, Homo sapiens
  source 1..48: mol_type = genomic DNA
ccctgaagcagcagccaggaacatgagctcttaccttgtcactcgggt

SEQ ID NO: 148 - DNA, 50 bp, Homo sapiens
  source 1..50: mol_type = genomic DNA
ctttgaggtgcttgtttgtgcctgtcctgggagagaccggcgcacagagg

SEQ ID NO: 149 - DNA, 46 bp, Homo sapiens
  source 1..46: mol_type = genomic DNA
cttcgagatgttctgagagctgaatgaggccttggaactcaaggat

SEQ ID NO: 150 - DNA, 50 bp, Homo sapiens
  source 1..50: mol_type = genomic DNA
ccgcgtccgcgccatggccatctgcaagcagtcacagcacatgacggagg

SEQ ID NO: 151 - DNA, 50 bp, Homo sapiens
  source 1..50: mol_type = genomic DNA
cacagggtaggtcttggccagttggcaaaacatcttgttgagggcagggg

SEQ ID NO: 152 - DNA, 50 bp, Homo sapiens
  source 1..50: mol_type = genomic DNA
ctttgaggtgcgtgtttatgcctgtcctgggagagaccggcgcacagagg

SEQ ID NO: 153 - DNA, 50 bp, Homo sapiens
  source 1..50: mol_type = genomic DNA
cattgacggcccccactgcgtcaagacctgcccggcagtagtcatgggag

SEQ ID NO: 154 - DNA, 50 bp, Homo sapiens
  source 1..50: mol_type = genomic DNA
ctttgaggtgcgtgtttgtgcctgtcctgggacagaccggcgcacagagg

SEQ ID NO: 155 - DNA, 50 bp, Homo sapiens
  source 1..50: mol_type = genomic DNA
ctttgaggtgcatgtttgtgcctgtcctgggagagaccggcgcacagagg

SEQ ID NO: 156 - DNA, 50 bp, Homo sapiens
  source 1..50: mol_type = genomic DNA
ctttgaggtgcgtgtttgtgcctgtcctgggagagactggcgcacagagg

SEQ ID NO: 157 - DNA, 50 bp, Homo sapiens
  source 1..50: mol_type = genomic DNA
cactgccacagctccttctctgagtggtaaaggcaatcctgaggaagagg

SEQ ID NO: 158 - DNA, 47 bp, Homo sapiens
  source 1..47: mol_type = genomic DNA
cagtgggtgttggggttggggtcaccgtggtggtggtggtaatgggt

SEQ ID NO: 159 - DNA, 47 bp, Homo sapiens
  source 1..47: mol_type = genomic DNA
cggtgggtgttggggttggggtcaccgtggtggtggtggtgatgggt

SEQ ID NO: 160 - DNA, 49 bp, Homo sapiens
  source 1..49: mol_type = genomic DNA
cctggaacttggtctcaaagattccagaagtcaggacacagcagcgaag

SEQ ID NO: 161 - DNA, 50 bp, Homo sapiens
  source 1..50: mol_type = genomic DNA
ctttgaggtgcgtgtttgtgcctgtcctgggaaagaccggcgcacagagg

SEQ ID NO: 162 - DNA, 50 bp, Homo sapiens
  source 1..50: mol_type = genomic DNA
ctttgagatgcgtgtttgtgcctgtcctgggagagaccggcgcacagagg

SEQ ID NO: 163 - DNA, 47 bp, Homo sapiens
  source 1..47: mol_type = genomic DNA
cactgccacagctccttctctgagtggtaaaggcaatcctgaggaag

SEQ ID NO: 164 - DNA, 48 bp, Homo sapiens
  source 1..48: mol_type = genomic DNA
cgacgccggcaaggccgccgcactcatttcctctccctccgcgcggag

SEQ ID NO: 165 - DNA, 48 bp, Homo sapiens
  source 1..48: mol_type = genomic DNA
cctgggcgctcgtgaagatggagccatattcctgcaggcgctctggag

SEQ ID NO: 166 - DNA, 48 bp, Homo sapiens
  source 1..48: mol_type = genomic DNA
cttggaggatgggccggtgaggccatcgctctggtggagagagggaag

SEQ ID NO: 167 - DNA, 46 bp, Homo sapiens
  source 1..46: mol_type = genomic DNA
ccatgcgggtgtcatctgagcacaggttccggaagtacctgggagt

SEQ ID NO: 168 - DNA, 48 bp, Homo sapiens
  source 1..48: mol_type = genomic DNA
cggggtggcgtgaccggtggatgttgaggaagggctggtgacatgaag

SEQ ID NO: 169 - DNA, 49 bp, Homo sapiens
  source 1..49: mol_type = genomic DNA
cggagggagaggaaatgagtgcggcggccttgccggcgtcggagcgggg

SEQ ID NO: 170 - DNA, 49 bp, Homo sapiens
  source 1..49: mol_type = genomic DNA
cgtagacctgggtccctcgcaccatgcggaggttgggcaatggtagagt

SEQ ID NO: 171 - DNA, 48 bp, Homo sapiens
  source 1..48: mol_type = genomic DNA
ctgggagagaccggcgcacaaaggaagagaatctccgcaagaaagggg

SEQ ID NO: 172 - DNA, 50 bp, Homo sapiens
  source 1..50: mol_type = genomic DNA
ctgggagagaccggcgcacaaaggaagagaatctccgcaagaaaggggag

SEQ ID NO: 173 - DNA, 49 bp, Homo sapiens
  source 1..49: mol_type = genomic DNA
cggagtcccatcctcaccatcatcacactggaagactccaggtcaggag

SEQ ID NO: 174 - DNA, 48 bp, Homo sapiens
  source 1..48: mol_type = genomic DNA
ctgggagagaccggcgcacagagaaagagaatctccgcaagaaagggg

SEQ ID NO: 175 - DNA, 50 bp, Homo sapiens
  source 1..50: mol_type = genomic DNA
cactgagctgtccctgggccagcagctgctgtgggccaccgctgatgagg

SEQ ID NO: 176 - DNA, 46 bp, Homo sapiens
  source 1..46: mol_type = genomic DNA
cctcggccctgccttctggccatacaggttctcggtggtgttgaag

SEQ ID NO: 177 - DNA, 50 bp, Homo sapiens
  source 1..50: mol_type = genomic DNA
ctgggagagaccggcgcacagagaaagagaatctccgcaagaaaggggag

SEQ ID NO: 178 - DNA, 50 bp, Homo sapiens
  source 1..50: mol_type = genomic DNA
cggagggagaggaaatgagtgcggcggccttgccggcgtcggagcggggt

SEQ ID NO: 179 - DNA, 48 bp, Homo sapiens
  source 1..48: mol_type = genomic DNA
ctgggagagactggcgcacagaggaagagaatctccgcaagaaagggg

SEQ ID NO: 180 - DNA, 50 bp, Homo sapiens
  source 1..50: mol_type = genomic DNA
ctaagtgaggtaagcaagcaggacaagaagcggtggaggagaccaagggt

SEQ ID NO: 181 - DNA, 47 bp, Homo sapiens
  source 1..47: mol_type = genomic DNA
cgctgaggaggggccagacctaagagcaatcagtgaggaatcagagg

SEQ ID NO: 182 - DNA, 50 bp, Homo sapiens
  source 1..50: mol_type = genomic DNA
ctgggagagactggcgcacagaggaagagaatctccgcaagaaaggggag

SEQ ID NO: 183 - DNA, 46 bp, Homo sapiens
  source 1..46: mol_type = genomic DNA
ctcagagaaggagctgtggtagtggcaccaaaatggattccagagt

SEQ ID NO: 184 - DNA, 46 bp, Homo sapiens
  source 1..46: mol_type = genomic DNA
ctcagaggaggagctgtggtagtggcaccagaatggattccagagt

SEQ ID NO: 185 - DNA, 47 bp, Homo sapiens
  source 1..47: mol_type = genomic DNA
ccctgtgtatgagccgcacatcctccaggtagctcatcccctggaat

SEQ ID NO: 186 - DNA, 49 bp, Homo sapiens
  source 1..49: mol_type = genomic DNA
cccagggagcactaagtgaggtaagcaagcaggacaagaagcggtggag

SEQ ID NO: 187 - DNA, 46 bp, Homo sapiens
  source 1..46: mol_type = genomic DNA
ctcagagaaggagctgtggcagtggcaccagaatggattccagagt

SEQ ID NO: 188 - DNA, 47 bp, Homo sapiens
  source 1..47: mol_type = genomic DNA
cgaagagctaagcgacttctgaggagaccggaaaatgggaggcgggg

SEQ ID NO: 189 - DNA, 50 bp, Homo sapiens
  source 1..50: mol_type = genomic DNA
cgctgagggtggagctgagggtggaaggggagtgagcagacacacggagg

SEQ ID NO: 190 - DNA, 46 bp, Homo sapiens
  source 1..46: mol_type = genomic DNA
cgcggagggagaggaaatgagtgcggcggccttgccggcgtcggag

SEQ ID NO: 191 - DNA, 50 bp, Homo sapiens
  source 1..50: mol_type = genomic DNA
cggggagccaatggttcagaaacaaattgagtgggttctaatcatggaat

SEQ ID NO: 192 - DNA, 49 bp, Homo sapiens
  source 1..49: mol_type = genomic DNA
cgctgagggtggagctgagggtggaaggggagtgagcagacacacggag

SEQ ID NO: 193 - DNA, 47 bp, Homo sapiens
  source 1..47: mol_type = genomic DNA
ctgggacggaacagctttgagatgcgtgtttgtgcctgtcctgggag

SEQ ID NO: 194 - DNA, 47 bp, Homo sapiens
  source 1..47: mol_type = genomic DNA
ctgggacggaacagctttgaggtgtgtgtttgtgcctgtcctgggag

SEQ ID NO: 195 - DNA, 47 bp, Homo sapiens
  source 1..47: mol_type = genomic DNA
ctgggacggaacagctttgaggtgcttgtttgtgcctgtcctgggag

SEQ ID NO: 196 - DNA, 47 bp, Homo sapiens
  source 1..47: mol_type = genomic DNA
ctgggacggaacagctttgaggtgcatgtttgtgcctgtcctgggag

SEQ ID NO: 197 - DNA, 47 bp, Homo sapiens
  source 1..47: mol_type = genomic DNA
ctgggacggaacagctttgaggtgcgtgtttatgcctgtcctgggag

SEQ ID NO: 198 - DNA, 46 bp, Homo sapiens
  source 1..46: mol_type = genomic DNA
cgtggcctgtggatactgaggaagtgtcggtgacaggaagaggggt

SEQ ID NO: 199 - DNA, 49 bp, Homo sapiens
  source 1..49: mol_type = genomic DNA
cggtgaggccatcgctctggtggagagagggaagaaaggaggagtgggg

SEQ ID NO: 200 - DNA, 47 bp, Homo sapiens
  source 1..47: mol_type = genomic DNA
ctgagggtggagctgagggtggaaggggagtgagcagacacacggag

SEQ ID NO: 201 - DNA, 48 bp, Homo sapiens
  source 1..48: mol_type = genomic DNA
ctgagggtggagctgagggtggaaggggagtgagcagacacacggagg

SEQ ID NO: 202 - DNA, 50 bp, Homo sapiens
  source 1..50: mol_type = genomic DNA
ctgtgactccatagaaaatctttctcctgcgcagtgatttcagagagagg

SEQ ID NO: 203 - DNA, 48 bp, Homo sapiens
  source 1..48: mol_type = genomic DNA
cagggagcactaagtgaggtaagcaagcaggacaagaagcggtggagg

SEQ ID NO: 204 - DNA, 50 bp, Homo sapiens
  source 1..50: mol_type = genomic DNA
ctgtgactccatagaaaatctttctcctgcttagtgatttcagagagagg

SEQ ID NO: 205 - DNA, 50 bp, Homo sapiens
  source 1..50: mol_type = genomic DNA
cagggagcactaagtgaggtaagcaagcaggacaagaagcggtggaggag

SEQ ID NO: 206 - DNA, 50 bp, Homo sapiens
  source 1..50: mol_type = genomic DNA
cgccgaccccgccactctcacctgacccgtgcacgacgctgcccgggagg

SEQ ID NO: 207 - DNA, 49 bp, Homo sapiens
  source 1..49: mol_type = genomic DNA
cttggagtaagtggagaagtatttgacttcagtcagcgacggaaagagt

SEQ ID NO: 208 - DNA, 47 bp, Homo sapiens
  source 1..47: mol_type = genomic DNA
cagggagcactaagtgaggtaagcaagcaggacaagaagcggtggag

SEQ ID NO: 209 - DNA, 50 bp, Homo sapiens
  source 1..50: mol_type = genomic DNA
ctgtgactccatagaaaatctttctcccgctcagtgatttcagagagagg

SEQ ID NO: 210 - DNA, 50 bp, Homo sapiens
  source 1..50: mol_type = genomic DNA
ctgtgactccatagaaaatctttctcctgctcagtgattttagagagagg

SEQ ID NO: 211 - DNA, 50 bp, Homo sapiens
  source 1..50: mol_type = genomic DNA
ctttgaggtgtgtgtttgtgcctgtcctgggagagaccggcgcacagagg

SEQ ID NO: 212 - DNA, 50 bp, Homo sapiens
  source 1..50: mol_type = genomic DNA
cccagggagcactaagtgaggtaagcaagcaggacaagaagcggtggagg

SEQ ID NO: 213 - DNA, 50 bp, Homo sapiens
  source 1..50: mol_type = genomic DNA
cggaggggtcaaccacttccctgggagctccctggactggagccgggagg

SEQ ID NO: 214 - DNA, 47 bp, Homo sapiens
  source 1..47: mol_type = genomic DNA
cgctgtgtcctttcaggatggtggatgtggggggcctgcggtcggag

SEQ ID NO: 215 - DNA, 50 bp, Homo sapiens
  source 1..50: mol_type = genomic DNA
cactgagccaccctacagccagaagagatatgaggaaatcgttaaggaag

SEQ ID NO: 216 - DNA, 47 bp, Homo sapiens
  source 1..47: mol_type = genomic DNA
ctgggacagccaagtctgtgacttgcactgtcagttgccctgagggg

SEQ ID NO: 217 - DNA, 49 bp, Homo sapiens
  source 1..49: mol_type = genomic DNA
cggaggggtcaaccacttccctgggagctccctggactggagccgggag

SEQ ID NO: 218 - DNA, 46 bp, Homo sapiens
  source 1..46: mol_type = genomic DNA
cacggaggtgtcttgagattatcatccgctgagggtggaaggggat

SEQ ID NO: 219 - DNA, 49 bp, Homo sapiens
  source 1..49: mol_type = genomic DNA
cgccgaccccgccactctcacctgacccgtgcacgacgctgcccgggag

SEQ ID NO: 220 - DNA, 50 bp, Homo sapiens
  source 1..50: mol_type = genomic DNA
ctatggtgtgttttttcattaatgacaaaaaaaaaaggtttcaactggat

SEQ ID NO: 221 - DNA, 50 bp, Homo sapiens
  source 1..50: mol_type = genomic DNA
cattgcagccttgatttattttggagtaagaaaaaaaaaagaatggggat

SEQ ID NO: 222 - DNA, 47 bp, Homo sapiens
  source 1..47: mol_type = genomic DNA
ctttggaaacttgccccttatttaaaaaaaaaaagaaaaaaaagagt

SEQ ID NO: 223 - DNA, 48 bp, Homo sapiens
  source 1..48: mol_type = genomic DNA
cattgcagccttgatttattttggagtaagaaaaaaaaaagaatgggg

SEQ ID NO: 224 - DNA, 49 bp, Homo sapiens
  source 1..49: mol_type = genomic DNA
cactgctcacctggagtttcatctgctacttttttttcaaaacctggat

SEQ ID NO: 225 - DNA, 48 bp, Homo sapiens
  source 1..48: mol_type = genomic DNA
catagttggtttttttttatttggggcagtgggcatgttatggggagg

SEQ ID NO: 226 - DNA, 50 bp, Homo sapiens
  source 1..50: mol_type = genomic DNA
caatgtaggctttttttcttttttaaaaaaaaatacttgtaggccagaag

SEQ ID NO: 227 - DNA, 50 bp, Homo sapiens
  source 1..50: mol_type = genomic DNA
catagttggtttttttttatttggggcagtgggcatgttatggggagggg

SEQ ID NO: 228 - DNA, 49 bp, Homo sapiens
  source 1..49: mol_type = genomic DNA
cagagttggctttttttttccttctttaaatcagtagtctaacaaggat

SEQ ID NO: 229 - DNA, 49 bp, Homo sapiens
  source 1..49: mol_type = genomic DNA
cagcggagatcctctttaaaaaaaaatgcatatgcaatgtcccaagaat

SEQ ID NO: 230 - DNA, 48 bp, Homo sapiens
  source 1..48: mol_type = genomic DNA
ctttggtgtttcttcattaaaagaaaaaaaaaagtcatcaatgtgggt

SEQ ID NO: 231 - DNA, 49 bp, Homo sapiens
  source 1..49: mol_type = genomic DNA
ctgtgttaacttccaggttccccttattattatagtgccgcccccgggg

SEQ ID NO: 232 - DNA, 47 bp, Homo sapiens
  source 1..47: mol_type = genomic DNA
catagttggtttttttttatttggggcagtgggcatgttatggggag

SEQ ID NO: 233 - DNA, 49 bp, Homo sapiens
  source 1..49: mol_type = genomic DNA
caatggtctgcatgggttacaaatgactttttttttttttaacaggaat

SEQ ID NO: 234 - DNA, 48 bp, Homo sapiens
  source 1..48: mol_type = genomic DNA
caaagttgcctaaatccatttggaaatctttaaaaaaaaattggggat

SEQ ID NO: 235 - DNA, 50 bp, Homo sapiens
  source 1..50: mol_type = genomic DNA
caatgctagtcgttactactatgtctgtctgagaaaaaaaaaaattgaat

SEQ ID NO: 236 - DNA, 50 bp, Homo sapiens
  source 1..50: mol_type = genomic DNA
cagaggaaaacagccaaagaaggaagaggaggaaaaggaaaaaaaagggg

SEQ ID NO: 237 - DNA, 50 bp, Homo sapiens
  source 1..50: mol_type = genomic DNA
cttgggtgaagagaaagaagctttttaagagtggaagaaaaaaaaagaag

SEQ ID NO: 238 - DNA, 48 bp, Homo sapiens
  source 1..48: mol_type = genomic DNA
cttggctgtccttaaaaaaaggttaaggaaaaagaggaaaagaagaag

SEQ ID NO: 239 - DNA, 49 bp, Homo sapiens
  source 1..49: mol_type = genomic DNA
ctctgttaaccagcttatgtccagcagagctggggggtgcaacccgggg

SEQ ID NO: 240 - DNA, 48 bp, Homo sapiens
  source 1..48: mol_type = genomic DNA
caatgtggacctgattctggccacacccccttcagccgcctggagaag

SEQ ID NO: 241 - DNA, 46 bp, Homo sapiens
  source 1..46: mol_type = genomic DNA
ctgtggagagtaacaacagagtgtatcagactccaaaaaaatgaat

SEQ ID NO: 242 - DNA, 47 bp, Homo sapiens
  source 1..47: mol_type = genomic DNA
catgggttgtttattctcgctctctctctttttttttttttgagagg

SEQ ID NO: 243 - DNA, 50 bp, Homo sapiens
  source 1..50: mol_type = genomic DNA
catgggttgtttattctcgctctctctctttttttttttttgagagggag

SEQ ID NO: 244 - DNA, 49 bp, Homo sapiens
  source 1..49: mol_type = genomic DNA
caacgtaaaaatgtaaatataaatttggttgagatctggaggggggagg

SEQ ID NO: 245 - DNA, 50 bp, Homo sapiens
  source 1..50: mol_type = genomic DNA
cttggtaaatttctactttcctccacatttttttttttaaagaaaggaat

SEQ ID NO: 246 - DNA, 47 bp, Homo sapiens
  source 1..47: mol_type = genomic DNA
ccttgctcactttttttactaaatgaaagtgatgatgatgatcgaat

SEQ ID NO: 247 - DNA, 48 bp, Homo sapiens
  source 1..48: mol_type = genomic DNA
ctttgcagccatgttgttttttttttctttttttttttcttggagaag

SEQ ID NO: 248 - DNA, 46 bp, Homo sapiens
  source 1..46: mol_type = genomic DNA
ctgaggaaagtaaattttttttttttaattactgggtttttagggt

SEQ ID NO: 249 - DNA, 47 bp, Homo sapiens
  source 1..47: mol_type = genomic DNA
ctcagtttccttgtttcttttgatttttttttcctaattgtgtgagg

SEQ ID NO: 250 - DNA, 48 bp, Homo sapiens
  source 1..48: mol_type = genomic DNA
cacaggagtgtgtagcaggataacagtctttttttttaatgacaggat

SEQ ID NO: 251 - DNA, 50 bp, Homo sapiens
  source 1..50: mol_type = genomic DNA
cattgtaaatgtgcttttaaaaaaaatactgatgttcctagtgaaagagg

SEQ ID NO: 252 - DNA, 49 bp, Homo sapiens
  source 1..49: mol_type = genomic DNA
cactgtttgttttacttccccaaaatggacctttttttttctaaagagt

SEQ ID NO: 253 - DNA, 46 bp, Homo sapiens
  source 1..46: mol_type = genomic DNA
caaagttgcctaaatccatttggaaatctttaaaaaaaaattgggg

SEQ ID NO: 254 - DNA, 50 bp, Homo sapiens
  source 1..50: mol_type = genomic DNA
ctctgttaaccagcttatgtccagcagagctggggggtgcaacccggggg

SEQ ID NO: 255 - DNA, 46 bp, Homo sapiens
  source 1..46: mol_type = genomic DNA
ctttgtaacctttttttgttttgttttttttttaaatattagggat

SEQ ID NO: 256 - DNA, 49 bp, Homo sapiens
  source 1..49: mol_type = genomic DNA
ccatgcttatgtttataagttttgagatttttttttttctgaaaaggat

SEQ ID NO: 257 - DNA, 47 bp, Homo sapiens
  source 1..47: mol_type = genomic DNA
caaagctggggcggttcctgtcaaaaaatactcattgcgcaaagggt

SEQ ID NO: 258 - DNA, 50 bp, Homo sapiens
  source 1..50: mol_type = genomic DNA
cgggggtgaacaggaatggagcattgagcttttggggaaaaaaaaagagt

SEQ ID NO: 259 - DNA, 46 bp, Homo sapiens
  source 1..46: mol_type = genomic DNA
caatgctttctactcatttttctatactttttttttgaggcagagt

SEQ ID NO: 260 - DNA, 50 bp, Homo sapiens
  source 1..50: mol_type = genomic DNA
caaagttggacaaagacttgagagatgcttttttttcccccagtgagggg

SEQ ID NO: 261 - DNA, 48 bp, Homo sapiens
  source 1..48: mol_type = genomic DNA
caaagttggacaaagacttgagagatgcttttttttcccccagtgagg

SEQ ID NO: 262 - DNA, 48 bp, Homo sapiens
  source 1..48: mol_type = genomic DNA
ctacgtaatttttttttttaatctaagcatttcttaactgagaggggt

SEQ ID NO: 263 - DNA, 50 bp, Homo sapiens
  source 1..50: mol_type = genomic DNA
ctgtgctcgcgctatctctctttctggcctggaggctatccagcgtgagt

SEQ ID NO: 264 - DNA, 47 bp, Homo sapiens
  source 1..47: mol_type = genomic DNA
ctacgtaatttttttttttaatctaagcatttcttaactgagagggg

SEQ ID NO: 265 - DNA, 49 bp, Homo sapiens
  source 1..49: mol_type = genomic DNA
cgctgctctcttctttgctctccagacggcttttttcgccaacatggat

SEQ ID NO: 266 - DNA, 49 bp, Homo sapiens
  source 1..49: mol_type = genomic DNA
caggggagggcagggggccaaaggagacacccccaagggcctccgggat

SEQ ID NO: 267 - DNA, 49 bp, Homo sapiens
  source 1..49: mol_type = genomic DNA
cggaggagcttgggggtacagggacttcagaggcggccaaaaaaggggt

SEQ ID NO: 268 - DNA, 48 bp, Homo sapiens
  source 1..48: mol_type = genomic DNA
caacgtaaaaatgtaaatataaatttggttgagatctggaggggggag

SEQ ID NO: 269 - DNA, 49 bp, Homo sapiens
  source 1..49: mol_type = genomic DNA
ctttgctaaagcacatcaaaaaaaggccaagatgagagaacaagagagg

SEQ ID NO: 270 - DNA, 50 bp, Homo sapiens
  source 1..50: mol_type = genomic DNA
caagggattttttttttaacaaaatgggaaagacctatgcagaaaggaag

SEQ ID NO: 271 - DNA, 50 bp, Homo sapiens
  source 1..50: mol_type = genomic DNA
cagaggatcttttttatattgataaatcagaggcagtgtttttttagagg

SEQ ID NO: 272 - DNA, 47 bp, Homo sapiens
  source 1..47: mol_type = genomic DNA
ctcggctctgctgcggcgggggatgctccaggagacgctaagcgagg

SEQ ID NO: 273 - DNA, 51 bp, Homo sapiens
  source 1..51: mol_type = genomic DNA
cggagatgttttgatagcgacgggaattttaactttctcaccttctgggat

SEQ ID NO: 274 - DNA, 50 bp, Homo sapiens
  source 1..50: mol_type = genomic DNA
cctagcagatgtggctcctaccccccaaagacccctgcccggaaacgggg

SEQ ID NO: 275 - DNA, 49 bp, Homo sapiens
  source 1..49: mol_type = genomic DNA
ctatgtatccttcctgattcatgacattaaaaaaaaaagcttaaagaag

SEQ ID NO: 276 - DNA, 50 bp, Homo sapiens
  source 1..50: mol_type = genomic DNA
ctgggcaggggtggctcctggggccttaggcccaagcccgggctctgggg

SEQ ID NO: 277 - DNA, 48 bp, Homo sapiens
  source 1..48: mol_type = genomic DNA
cggaggagcttgggggtacagggacttcagaggcggccaaaaaagggg

SEQ ID NO: 278 - DNA, 46 bp, Homo sapiens
  source 1..46: mol_type = genomic DNA
cgaggcttaatggaagaactggttagcattttttttttttgagggt

SEQ ID NO: 279 - DNA, 48 bp, Homo sapiens
  source 1..48: mol_type = genomic DNA
caggggtctttggggggtaggagccacatctgctaggcgaggaggagg

SEQ ID NO: 280 - DNA, 50 bp, Homo sapiens
  source 1..50: mol_type = genomic DNA
ccaaggaagattttgacagtctcttgcaatcggctaaaaaaagagtgggt

SEQ ID NO: 281 - DNA, 50 bp, Homo sapiens
  source 1..50: mol_type = genomic DNA
ctctgcttatttataggactgattgtgtagaaaaaaagacagccctgaag

SEQ ID NO: 282 - DNA, 47 bp, Homo sapiens
  source 1..47: mol_type = genomic DNA
cgtaggtgttctcccagtaggccttgagggctggcgtgccggggggt

SEQ ID NO: 283 - DNA, 46 bp, Homo sapiens
  source 1..46: mol_type = genomic DNA
cctagataagcagaattccaatgttttttaagtacttctcgggggt

SEQ ID NO: 284 - DNA, 49 bp, Homo sapiens
  source 1..49: mol_type = genomic DNA
cccagtaggagtgaaggggatttttttttcttttaaactgaaggtgggg

SEQ ID NO: 285 - DNA, 46 bp, Homo sapiens
  source 1..46: mol_type = genomic DNA
ctcagcaatgtctttttttttcggccttcgaggtcacgttaaggag

SEQ ID NO: 286 - DNA, 46 bp, Homo sapiens
  source 1..46: mol_type = genomic DNA
ccctgcttctctgtcatgatccccccaatgactcccgggccaggag

SEQ ID NO: 287 - DNA, 49 bp, Homo sapiens
  source 1..49: mol_type = genomic DNA
ctctgtacacgtatctgagatctcaggctccttttttgatgctgtgagt

SEQ ID NO: 288 - DNA, 46 bp, Homo sapiens
  source 1..46: mol_type = genomic DNA
ctgagttaaaatgtgaagggattttttttttcagattactgagagt

SEQ ID NO: 289 - DNA, 50 bp, Homo sapiens
  source 1..50: mol_type = genomic DNA
ctgtggaagagactggcactcccgggcacaggggggcagtgctggggggt

SEQ ID NO: 290 - DNA, 47 bp, Homo sapiens
  source 1..47: mol_type = genomic DNA
ccctggacttttcaagcattttttttgacaattaaattgggttggat

SEQ ID NO: 291 - DNA, 49 bp, Homo sapiens
  source 1..49: mol_type = genomic DNA
caaagaaacacccacctcctgtggaaacaaaaaaatccttggattgaat

SEQ ID NO: 292 - DNA, 47 bp, Homo sapiens
  source 1..47: mol_type = genomic DNA
caggggtctttggggggtaggagccacatctgctaggcgaggaggag

SEQ ID NO: 293 - DNA, 50 bp, Homo sapiens
  source 1..50: mol_type = genomic DNA
ctgtgtaaaaaaatcatgatgaggtgctgtgctctctgtatgaaatgggg

SEQ ID NO: 294 - DNA, 48 bp, Homo sapiens
  source 1..48: mol_type = genomic DNA
caaggccaaattgaacgagtcattaaggaaaaaaagcagtggaagaag

SEQ ID NO: 295 - DNA, 47 bp, Homo sapiens
  source 1..47: mol_type = genomic DNA
ccagggaaaggagcccccctgtttcctgcagtgtttccaggggggat

SEQ ID NO: 296 - DNA, 48 bp, Homo sapiens
  source 1..48: mol_type = genomic DNA
cccgggatgcctgcctctaaaaaatgcagggtgaacgcggtggaggag

SEQ ID NO: 297 - DNA, 49 bp, Homo sapiens
  source 1..49: mol_type = genomic DNA
cacaggacgtccctgctctcctggctgcagactagagtggggagagagg

SEQ ID NO: 298 - DNA, 46 bp, Homo sapiens
  source 1..46: mol_type = genomic DNA
caatgttgtcgctgcagccccccagtgccagtcggggcccccgggg

SEQ ID NO: 299 - DNA, 46 bp, Homo sapiens
  source 1..46: mol_type = genomic DNA
caggggtggctacagtggagagggcttggggcgtactccggtgagt

SEQ ID NO: 300 - DNA, 46 bp, Homo sapiens
  source 1..46: mol_type = genomic DNA
caaaggtgaaaaaacaatgcattcttgctttaaaaaaaaaaagaag

SEQ ID NO: 301 - DNA, 46 bp, Homo sapiens
  source 1..46: mol_type = genomic DNA
ccctgctctcctggctgcagactagagtggggagagaggagagggt

SEQ ID NO: 302 - DNA, 49 bp, Homo sapiens
  source 1..49: mol_type = genomic DNA
ctcgggaccctaaaatccctaagagcaagcgccaaaaaaggaggtgagt

SEQ ID NO: 303 - DNA, 46 bp, Homo sapiens
  source 1..46: mol_type = genomic DNA
cacggctactttttttaaagatacctataatatagaaatcaaggag

SEQ ID NO: 304 - DNA, 50 bp, Homo sapiens
  source 1..50: mol_type = genomic DNA
cagagctgctactttatatctgtatatagttttgctttttttggtagggg

SEQ ID NO: 305 - DNA, 48 bp, Homo sapiens
  source 1..48: mol_type = genomic DNA
catcgtattttgttccctttttttgttttgttttggtaatgaaagagg

SEQ ID NO: 306 - DNA, 49 bp, Homo sapiens
  source 1..49: mol_type = genomic DNA
ctcgggaagccaggaggaaggagcggccagccaggacccccccaggagg

SEQ ID NO: 307 - DNA, 48 bp, Homo sapiens
  source 1..48: mol_type = genomic DNA
cagtgtaatttcatggggtttcccccccccaataatttcgcctagagt

SEQ ID NO: 308 - DNA, 49 bp, Homo sapiens
  source 1..49: mol_type = genomic DNA
cactggaacacacgctggccctcctccggtcccggcacccactgggggt

SEQ ID NO: 309 - DNA, 50 bp, Homo sapiens
  source 1..50: mol_type = genomic DNA
ctttgaaacatcaaacacttctttaagcttttttttttaatgtcatgaat

SEQ ID NO: 310 - DNA, 48 bp, Homo sapiens
  source 1..48: mol_type = genomic DNA
ctaagcactgccagggggcgtcccgtgtgagtcggtgaacgagcgagg

SEQ ID NO: 311 - DNA, 48 bp, Homo sapiens
  source 1..48: mol_type = genomic DNA
cctcgcttagcgtctcctggagcatcccccgccgcagcagagccgagt

SEQ ID NO: 312 - DNA, 50 bp, Homo sapiens
  source 1..50: mol_type = genomic DNA
cctcgctcagtccgggggtatcaccaacatggtggctcctagttcagggg

SEQ ID NO: 313 - DNA, 49 bp, Homo sapiens
  source 1..49: mol_type = genomic DNA
ccctgtttcctgcagtgtttccaggggggatggtggtgcactcggggag

SEQ ID NO: 314 - DNA, 46 bp, Homo sapiens
  source 1..46: mol_type = genomic DNA
ctgtgcatccatgttggcgaaaaaagccgtctggagagcaaagaag

SEQ ID NO: 315 - DNA, 48 bp, Homo sapiens
  source 1..48: mol_type = genomic DNA
ccctgcattttttttatgatggctcaacagcaaaggtgtctggaggag

SEQ ID NO: 316 - DNA, 49 bp, Homo sapiens
  source 1..49: mol_type = genomic DNA
caaggaagattttgacagtctcttgcaatcggctaaaaaaagagtgggt

SEQ ID NO: 317 - DNA, 49 bp, Homo sapiens
  source 1..49: mol_type = genomic DNA
ctgagctggtcatgttgtcatggagctgacaaaaaaaaaagtggagggg

SEQ ID NO: 318 - DNA, 50 bp, Homo sapiens
  source 1..50: mol_type = genomic DNA
cccagtaggagtgaaggggatttttttttcttttaaactgaaggtggggt

SEQ ID NO: 319 - DNA, 49 bp, Homo sapiens
  source 1..49: mol_type = genomic DNA
ctcagcacaacacctctacttcccagattttttttttcaaactctgaag

SEQ ID NO: 320 - DNA, 46 bp, Homo sapiens
  source 1..46: mol_type = genomic DNA
ccaaggaagattttgacagtctcttgcaatcggctaaaaaaagagt

SEQ ID NO: 321 - DNA, 48 bp, Homo sapiens
  source 1..48: mol_type = genomic DNA
cctggcagcatgttccagctcttgatgtttttaaactttttttagaag

SEQ ID NO: 322 - DNA, 48 bp, Homo sapiens
  source 1..48: mol_type = genomic DNA
caaagttttcagtatcaccaattatggcttaaaaagaaaaaaaaggag

SEQ ID NO: 323 - DNA, 46 bp, Homo sapiens
  source 1..46: mol_type = genomic DNA
ccaggttccacttactcttcataaaactgattttttttgccagaat

SEQ ID NO: 324 - DNA, 47 bp, Homo sapiens
  source 1..47: mol_type = genomic DNA
caatgtagcaaaatcaaacttaaaaaaaaaaagaagaaaagaagaag

SEQ ID NO: 325 - DNA, 48 bp, Homo sapiens
  source 1..48: mol_type = genomic DNA
caaggggggactgatgcagtgtgaggaattgatagcgtatctgcgggt

SEQ ID NO: 326 - DNA, 48 bp, Homo sapiens
  source 1..48: mol_type = genomic DNA
ctcgggaagccaggaggaaggagcggccagccaggacccccccaggag

SEQ ID NO: 327 - DNA, 46 bp, Homo sapiens
  source 1..46: mol_type = genomic DNA
ctcagtaatctgaaaaaaaaaatcccttcacattttaactcaggag

SEQ ID NO: 328 - DNA, 48 bp, Homo sapiens
  source 1..48: mol_type = genomic DNA
ctcggatcaataaatttgttgcttcatattccgacataaaaaaagaag

SEQ ID NO: 329 - DNA, 46 bp, Homo sapiens
  source 1..46: mol_type = genomic DNA
cccgggatgcctgcctctaaaaaatgcagggtgaacgcggtggagg

SEQ ID NO: 330 - DNA, 49 bp, Homo sapiens
  source 1..49: mol_type = genomic DNA
caatgatcttagaagtactgaaaaaaaagacgtttttaaaacgtagagg

SEQ ID NO: 331 - DNA, 50 bp, Homo sapiens
  source 1..50: mol_type = genomic DNA
ctcggcaggttgctgtttttttggtggtctgtctatcaagaaggatgaag

SEQ ID NO: 332 - DNA, 46 bp, Homo sapiens
  source 1..46: mol_type = genomic DNA
ctgagttcagaagtagcattcccgtgtacaaaaaaggtggaagaat

SEQ ID NO: 333 - DNA, 46 bp, Homo sapiens
  source 1..46: mol_type = genomic DNA
ccgagttggggtttgttttgttttgatttttttttttaaagcgggt

SEQ ID NO: 334 - DNA, 50 bp, Homo sapiens
  source 1..50: mol_type = genomic DNA
catggaacggtggggggtgcgcttgattctacttcaggaggcacatgggg

SEQ ID NO: 335 - DNA, 49 bp, Homo sapiens
  source 1..49: mol_type = genomic DNA
ccgggcaggggtctttggggggtaggagccacatctgctaggcgaggag

SEQ ID NO: 336 - DNA, 49 bp, Homo sapiens
  source 1..49: mol_type = genomic DNA
ctgtgtatgaatatgacagtatttatgatgaaatgcagaaaaaaaggag

SEQ ID NO: 337 - DNA, 47 bp, Homo sapiens
  source 1..47: mol_type = genomic DNA
caaagaatatctcaaaaaacaccaaaacaaatttttttcccctggag

SEQ ID NO: 338 - DNA, 50 bp, Homo sapiens
  source 1..50: mol_type = genomic DNA
cagaggctggcagaggtgcagggggggactgccatctggggcactagaat

SEQ ID NO: 339 - DNA, 46 bp, Homo sapiens
  source 1..46: mol_type = genomic DNA
cacagttctatatagagctttttttttcttgttgcttaagctggag

SEQ ID NO: 340 - DNA, 47 bp, Homo sapiens
  source 1..47: mol_type = genomic DNA
catggttgtaaataaaggtttctcttttttttcctagtcttttgagt

SEQ ID NO: 341 - DNA, 49 bp, Homo sapiens
  source 1..49: mol_type = genomic DNA
cccagctcctaggacttattaaaaaaaaatgacattagatttatggagg

SEQ ID NO: 342 - DNA, 50 bp, Homo sapiens
  source 1..50: mol_type = genomic DNA
ctgtgccttcctcaccccgtttccgggcaggggtctttggggggtaggag

SEQ ID NO: 343 - DNA, 47 bp, Homo sapiens
  source 1..47: mol_type = genomic DNA
catcggaagatgcgagtttgtgccttttttttattgctctggtggat

SEQ ID NO: 344 - DNA, 49 bp, Homo sapiens
  source 1..49: mol_type = genomic DNA
caatgccaagcaccacggcaatggcaccccctgcaccacaagcaggggg

SEQ ID NO: 345 - DNA, 49 bp, Homo sapiens
  source 1..49: mol_type = genomic DNA
cacagttgctagggattgggagattaattgggtaaaaaaaaaaatgaag

SEQ ID NO: 346 - DNA, 47 bp, Homo sapiens
  source 1..47: mol_type = genomic DNA
ccctgtttcctgcagtgtttccaggggggatggtggtgcactcgggg

SEQ ID NO: 347 - DNA, 49 bp, Homo sapiens
  source 1..49: mol_type = genomic DNA
cctcgtacttccacactcggctctgctgcggcgggggatgctccaggag

SEQ ID NO: 348 - DNA, 50 bp, Homo sapiens
  source 1..50: mol_type = genomic DNA
ccctgtttcctgcagtgtttccaggggggatggtggtgcactcggggagg

SEQ ID NO: 349 - DNA, 49 bp, Homo sapiens
  source 1..49: mol_type = genomic DNA
ctttggggggtaggagccacatctgctaggcgaggaggaggaagggggg

SEQ ID NO: 350 - DNA, 50 bp, Homo sapiens
  source 1..50: mol_type = genomic DNA
ccgggcaggggtctttggggggtaggagccacatctgctaggcgaggagg

SEQ ID NO: 351 - DNA, 48 bp, Homo sapiens
  source 1..48: mol_type = genomic DNA
ctttgtaataggtctagacaaaaaaaaagaattttcattttgaaggat

SEQ ID NO: 352 - DNA, 46 bp, Homo sapiens
  source 1..46: mol_type = genomic DNA
cgccgtatatccaacattaaaaagaaaaaaaaggctgtttaagaat

SEQ ID NO: 353 - DNA, 47 bp, Homo sapiens
  source 1..47: mol_type = genomic DNA
ctgagctggtcatgttgtcatggagctgacaaaaaaaaaagtggagg

SEQ ID NO: 354 - DNA, 46 bp, Homo sapiens
  source 1..46: mol_type = genomic DNA
caaggaagagaggaggccacggtgagaccacggatagctggggggt

SEQ ID NO: 355 - DNA, 50 bp, Homo sapiens
  source 1..50: mol_type = genomic DNA
cacagctgctcatccagaagatgaccggggatggaagtccaggcgggggt

SEQ ID NO: 356 - DNA, 49 bp, Homo sapiens
  source 1..49: mol_type = genomic DNA
ccacgcttaaagtaaccatgcaaccgactatagtcaaaaaaaaaaggag

SEQ ID NO: 357 - DNA, 49 bp, Homo sapiens
  source 1..49: mol_type = genomic DNA
ctacgttttaaaaacgtcttttttttcagtacttctaagatcattgaat

SEQ ID NO: 358 - DNA, 49 bp, Homo sapiens
  source 1..49: mol_type = genomic DNA
cacagttcagagatgggaaaaaaagtgggtgagaagctaagtgaaggag

SEQ ID NO: 359 - DNA, 48 bp, Homo sapiens
  source 1..48: mol_type = genomic DNA
cccagctcctaggacttattaaaaaaaaatgacattagatttatggag

SEQ ID NO: 360 - DNA, 48 bp, Homo sapiens
  source 1..48: mol_type = genomic DNA
ctatgccacactttttttttcccaccttaacattattagacacagagt

SEQ ID NO: 361 - DNA, 49 bp, Homo sapiens
  source 1..49: mol_type = genomic DNA
cagcggccaccatggccatgccagaggtaaaaaacgacggcggcggaag

SEQ ID NO: 362 - DNA, 46 bp, Homo sapiens
  source 1..46: mol_type = genomic DNA
ccctgcattttttttatgatggctcaacagcaaaggtgtctggagg

SEQ ID NO: 363 - DNA, 49 bp, Homo sapiens
  source 1..49: mol_type = genomic DNA
cagagtgtatcagactccaaaaaaatgaataatgtgtatgaggaagagg

SEQ ID NO: 364 - DNA, 50 bp, Homo sapiens
  source 1..50: mol_type = genomic DNA
cacagttgcccatgggaaaaaccaatggattttttttaagcaagatgaat

SEQ ID NO: 365 - DNA, 47 bp, Homo sapiens
  source 1..47: mol_type = genomic DNA
caaggccgaattaaaaaaaaatcttcattaaagaatttaatagggag

SEQ ID NO: 366 - DNA, 49 bp, Homo sapiens
  source 1..49: mol_type = genomic DNA
cacagatttgaggactaagaactttcacttttttttcctattatagaag

SEQ ID NO: 367 - DNA, 46 bp, Homo sapiens
  source 1..46: mol_type = genomic DNA
ccctggtcacccaggacagagcggaaaaaaaaaaagaggtcagggt

SEQ ID NO: 368 - DNA, 48 bp, Homo sapiens
  source 1..48: mol_type = genomic DNA
caaggccgaattaaaaaaaaatcttcattaaagaatttaatagggagt

SEQ ID NO: 369 - DNA, 47 bp, Homo sapiens
  source 1..47: mol_type = genomic DNA
ccgggcaggggtctttggggggtaggagccacatctgctaggcgagg

SEQ ID NO: 370 - DNA, 50 bp, Homo sapiens
  source 1..50: mol_type = genomic DNA
cgtcgtaggtgttctcccagtaggccttgagggctggcgtgccggggggt

SEQ ID NO: 371 - DNA, 50 bp, Homo sapiens
  source 1..50: mol_type = genomic DNA
ccacgcttaaagtaaccatgcaaccgactatagtcaaaaaaaaaaggagt

SEQ ID NO: 372 - DNA, 47 bp, Homo sapiens
  source 1..47: mol_type = genomic DNA
cattgtcaatttatgaacaagacaggattttttttttcccatggaat

SEQ ID NO: 373 - DNA, 50 bp, Homo sapiens
  source 1..50: mol_type = genomic DNA
cacggcaatggcaccccctgcaccacaagcagggggcactgtactgggag

SEQ ID NO: 374 - DNA, 48 bp, Homo sapiens
  source 1..48: mol_type = genomic DNA
caatgccaagcaccacggcaatggcaccccctgcaccacaagcagggg

SEQ ID NO: 375 - DNA, 46 bp, Homo sapiens
  source 1..46: mol_type = genomic DNA
ctcggcaggttgctgtttttttggtggtctgtctatcaagaaggat

SEQ ID NO: 376 - DNA, 49 bp, Homo sapiens
  source 1..49: mol_type = genomic DNA
cagagccacccccagcccacccaagaccaccagccctgagcctcaggag

SEQ ID NO: 377 - DNA, 50 bp, Homo sapiens
  source 1..50: mol_type = genomic DNA
ctttggggggtaggagccacatctgctaggcgaggaggaggaaggggggt

SEQ ID NO: 378 - DNA, 48 bp, Homo sapiens
  source 1..48: mol_type = genomic DNA
ctttggggggtaggagccacatctgctaggcgaggaggaggaaggggg

SEQ ID NO: 379 - DNA, 50 bp, Homo sapiens
  source 1..50: mol_type = genomic DNA
ccttgcagctggatttgcgacttttttttgtcttaaaatttttactggat

SEQ ID NO: 380 - DNA, 50 bp, Homo sapiens
  source 1..50: mol_type = genomic DNA
ctgtgtatgaatatgacagtatttatgatgaaatgcagaaaaaaaggagg

SEQ ID NO: 381 - DNA, 50 bp, Homo sapiens
  source 1..50: mol_type = genomic DNA
caaggaatatatgttgttgttgttgttttaaacccatttttttttagaat

SEQ ID NO: 382 - DNA, 46 bp, Homo sapiens
  source 1..46: mol_type = genomic DNA
ctgagctggtcatgttgtcatggagctgacaaaaaaaaaagtggag

SEQ ID NO: 383 - DNA, 50 bp, Homo sapiens
  source 1..50: mol_type = genomic DNA
ctacgccctgctgcgcgtggagcacggtccccccacaccaagaactggag

SEQ ID NO: 384 - DNA, 47 bp, Homo sapiens
  source 1..47: mol_type = genomic DNA
ctctgcacccccccaccaaccccagggatggggcgtcagggaaggag

SEQ ID NO: 385 - DNA, 46 bp, Homo sapiens
  source 1..46: mol_type = genomic DNA
ccatgatatggattgtgttttttttagcaccttattttccttgaag

SEQ ID NO: 386 - DNA, 49 bp, Homo sapiens
  source 1..49: mol_type = genomic DNA
catggccaatcccccttccccgtcaggactcacagctcttcaagggggg

SEQ ID NO: 387 - DNA, 50 bp, Homo sapiens
  source 1..50: mol_type = genomic DNA
ccaggatgtatttgccgttcggggagaacttcacaaaagacacggggggt

SEQ ID NO: 388 - DNA, 48 bp, Homo sapiens
  source 1..48: mol_type = genomic DNA
cgggggactggccaagggccagggagcccaggggtggctacagtggag

SEQ ID NO: 389 - DNA, 50 bp, Homo sapiens
  source 1..50: mol_type = genomic DNA
cctggcaaccccctgggaggtggcttacatggtggtgagcagaggggggt

SEQ ID NO: 390 - DNA, 49 bp, Homo sapiens
  source 1..49: mol_type = genomic DNA
cctgggaggtggcttacatggtggtgagcagaggggggtgtagtcgggg

SEQ ID NO: 391 - DNA, 49 bp, Homo sapiens
  source 1..49: mol_type = genomic DNA
ctgagattttcacaagctttttcccataaagactgcatttttttaggag

SEQ ID NO: 392 - DNA, 49 bp, Homo sapiens
  source 1..49: mol_type = genomic DNA
cttagtcacactaaattaaaaaaaaaattccttagggatatcttagagt

SEQ ID NO: 393 - DNA, 46 bp, Homo sapiens
  source 1..46: mol_type = genomic DNA
ccagggactacctcggcttttaatttaaaaaaaaaaagaagtgggt

SEQ ID NO: 394 - DNA, 50 bp, Homo sapiens
  source 1..50: mol_type = genomic DNA
cagagccacccccagcccacccaagaccaccagccctgagcctcaggagt

SEQ ID NO: 395 - DNA, 50 bp, Homo sapiens
  source 1..50: mol_type = genomic DNA
catgggcttttttttgaataaaaaagcagacaaatagactttctcgggat

SEQ ID NO: 396 - DNA, 47 bp, Homo sapiens
  source 1..47: mol_type = genomic DNA
ctttggggggtaggagccacatctgctaggcgaggaggaggaagggg

SEQ ID NO: 397 - DNA, 46 bp, Homo sapiens
  source 1..46: mol_type = genomic DNA
ctaagttcttagattttgggggattttttttttaaacgatgagaag

SEQ ID NO: 398 - DNA, 49 bp, Homo sapiens
  source 1..49: mol_type = genomic DNA
ctgggttgggaaacccattgctcgagtggttaaaaaaagaccggagaat

SEQ ID NO: 399 - DNA, 47 bp, Homo sapiens
  source 1..47: mol_type = genomic DNA
ccaaggaagagaggaggccacggtgagaccacggatagctggggggt

SEQ ID NO: 400 - DNA, 47 bp, Homo sapiens
  source 1..47: mol_type = genomic DNA
cacagatgccggggtgtgtgtgtgtgtgtgtattttcactgtggggt

SEQ ID NO: 401 - DNA, 48 bp, Homo sapiens
  source 1..48: mol_type = genomic DNA
ccaggaagactgtgagggtttttctttttttttttaagggccaagggt

SEQ ID NO: 402 - DNA, 46 bp, Homo sapiens
  source 1..46: mol_type = genomic DNA
cagtgcaacccccgcctggacttccatccccggtcatcttctggat

SEQ ID NO: 403 - DNA, 46 bp, Homo sapiens
  source 1..46: mol_type = genomic DNA
ccctgctgcgcgtggagcacggtccccccacaccaagaactggagg

SEQ ID NO: 404 - DNA, 50 bp, Homo sapiens
  source 1..50: mol_type = genomic DNA
cacagttttgggagtctttttttggacactttctccaggagggattgagg

SEQ ID NO: 405 - DNA, 47 bp, Homo sapiens
  source 1..47: mol_type = genomic DNA
caggggaaaaaaatttgttttggtgttttttgagatattctttggat

SEQ ID NO: 406 - DNA, 48 bp, Homo sapiens
  source 1..48: mol_type = genomic DNA
ccatggttgtaaataaaggtttctcttttttttcctagtcttttgagt

SEQ ID NO: 407 - DNA, 48 bp, Homo sapiens
  source 1..48: mol_type = genomic DNA
cacaggggggcagtgctggggggtacagggaactggactgtctcggat

SEQ ID NO: 408 - DNA, 50 bp, Homo sapiens
  source 1..50: mol_type = genomic DNA
ctaggatggggattcttgggacattgcatatgcatttttttttaaagagg

SEQ ID NO: 409 - DNA, 50 bp, Homo sapiens
  source 1..50: mol_type = genomic DNA
ccatgcaggaagagagtgtggtgagaagatgggtattcctttttttggag

SEQ ID NO: 410 - DNA, 47 bp, Homo sapiens
  source 1..47: mol_type = genomic DNA
cccagattttttttttcaaactctgaaggaagtgatgttagacggat

SEQ ID NO: 411 - DNA, 47 bp, Homo sapiens
  source 1..47: mol_type = genomic DNA
cctaggaaggacaaggccgaattaaaaaaaaatcttcattaaagaat

SEQ ID NO: 412 - DNA, 49 bp, Homo sapiens
  source 1..49: mol_type = genomic DNA
caatgggtctccttccctgacgccccatccctggggttggtgggggggt

SEQ ID NO: 413 - DNA, 47 bp, Homo sapiens
  source 1..47: mol_type = genomic DNA
catggccaatcccccttccccgtcaggactcacagctcttcaagggg

SEQ ID NO: 414 - DNA, 46 bp, Homo sapiens
  source 1..46: mol_type = genomic DNA
cacagatgccggggtgtgtgtgtgtgtgtgtattttcactgtgggg

SEQ ID NO: 415 - DNA, 47 bp, Homo sapiens
  source 1..47: mol_type = genomic DNA
cttagtttaaaaaaaaaagacttattttctagaaaacgttaatgggt

SEQ ID NO: 416 - DNA, 47 bp, Homo sapiens
  source 1..47: mol_type = genomic DNA
ccggggatggaagtccaggcgggggttgcactggagcgtcaaaggag

SEQ ID NO: 417 - DNA, 46 bp, Homo sapiens
  source 1..46: mol_type = genomic DNA
cagagtgtatcagactccaaaaaaatgaataatgtgtatgaggaag

SEQ ID NO: 418 - DNA, 50 bp, Homo sapiens
  source 1..50: mol_type = genomic DNA
cagggattagggatttgggttttttttttttctctttttaatactagaat

SEQ ID NO: 419 - DNA, 46 bp, Homo sapiens
  source 1..46: mol_type = genomic DNA
cacagttcagagatgggaaaaaaagtgggtgagaagctaagtgaag

SEQ ID NO: 420 - DNA, 49 bp, Homo sapiens
  source 1..49: mol_type = genomic DNA
ctgggttctgacagagaagctggggattggcagggggtggcatcggagg

SEQ ID NO: 421 - DNA, 49 bp, Homo sapiens
  source 1..49: mol_type = genomic DNA
cacagaaaagaaaaaaaaaaggaaaaaaaataaagtgtgtgccttgggt

SEQ ID NO: 422 - DNA, 48 bp, Homo sapiens
  source 1..48: mol_type = genomic DNA
catggccaatcccccttccccgtcaggactcacagctcttcaaggggg

SEQ ID NO: 423 - DNA, 49 bp, Homo sapiens
  source 1..49: mol_type = genomic DNA
catggaccaggtggccttctctctgcacccccccaccaaccccagggat

SEQ ID NO: 424 - DNA, 47 bp, Homo sapiens
  source 1..47: mol_type = genomic DNA
cagagccagggggtgggcatgaggggacatgcaggcaggcaccgggt

SEQ ID NO: 425 - DNA, 50 bp, Homo sapiens
  source 1..50: mol_type = genomic DNA
catagtgctatacaacttctccaggcggctgaagggggtgtggccagaat

SEQ ID NO: 426 - DNA, 47 bp, Homo sapiens
  source 1..47: mol_type = genomic DNA
ccgggatgcctgcctctaaaaaatgcagggtgaacgcggtggaggag

SEQ ID NO: 427 - DNA, 50 bp, Homo sapiens
  source 1..50: mol_type = genomic DNA
catggccaatcccccttccccgtcaggactcacagctcttcaaggggggg

SEQ ID NO: 428 - DNA, 47 bp, Homo sapiens
  source 1..47: mol_type = genomic DNA
ccaagtaagtatttttttttgtctttagcaaagtttagactgtgaat

SEQ ID NO: 429 - DNA, 49 bp, Homo sapiens
  source 1..49: mol_type = genomic DNA
ccttgcgcagttctgggttcatatctgggttggggggaaggggtagggt

SEQ ID NO: 430 - DNA, 47 bp, Homo sapiens
  source 1..47: mol_type = genomic DNA
ccctgcggaatttaaacctccaaaaaagcagctgctttcagaggagg

SEQ ID NO: 431 - DNA, 48 bp, Homo sapiens
  source 1..48: mol_type = genomic DNA
cagtgtttccaggggggatggtggtgcactcggggaggcgggaagagg

SEQ ID NO: 432 - DNA, 46 bp, Homo sapiens
  source 1..46: mol_type = genomic DNA
caacgtcaacatggctttcaccggcggcctggaccccccatgggag

SEQ ID NO: 433 - DNA, 46 bp, Homo sapiens
  source 1..46: mol_type = genomic DNA
cccagcagaagctgtgacccccccttcctccctggtgaggtcggag

SEQ ID NO: 434 - DNA, 49 bp, Homo sapiens
  source 1..49: mol_type = genomic DNA
caccgtgacctcaaactctttggactgtttgaaaaaaaaaaattggaag

SEQ ID NO: 435 - DNA, 48 bp, Homo sapiens
  source 1..48: mol_type = genomic DNA
ctctggggcaccgcgccttgggggggcccccatgactctggggtgggt

SEQ ID NO: 436 - DNA, 49 bp, Homo sapiens
  source 1..49: mol_type = genomic DNA
ctggggggccccaataaattacattcttgagagagcatagtgtgtgggg

SEQ ID NO: 437 - DNA, 50 bp, Homo sapiens
  source 1..50: mol_type = genomic DNA
ctttgtgcatttagttccgcatatgatggtttttttttacattaaagagt

SEQ ID NO: 438 - DNA, 49 bp, Homo sapiens
  source 1..49: mol_type = genomic DNA
ctgcgagctgtggtggtggatgactaccgtcggcgcaaaaaaagggagg

SEQ ID NO: 439 - DNA, 46 bp, Homo sapiens
  source 1..46: mol_type = genomic DNA
ctctgtacgtgtctacagcaaaacacgttttcgaaaaaaactgaag

SEQ ID NO: 440 - DNA, 47 bp, Homo sapiens
  source 1..47: mol_type = genomic DNA
cccaggttccacttactcttcataaaactgattttttttgccagaat

SEQ ID NO: 441 - DNA, 50 bp, Homo sapiens
  source 1..50: mol_type = genomic DNA
cctcggtcctaccccctgacctgcgctgcaactacagcatccgggtggag

SEQ ID NO: 442 - DNA, 47 bp, Homo sapiens
  source 1..47: mol_type = genomic DNA
ctgtggggagggcggtggggggtgccagcctgccatgcgtgcagggg

SEQ ID NO: 443 - DNA, 46 bp, Homo sapiens
  source 1..46: mol_type = genomic DNA
cggggggacactgagttcattaaggggggtgacatttcttcaggat

SEQ ID NO: 444 - DNA, 46 bp, Homo sapiens
  source 1..46: mol_type = genomic DNA
catggctttcaccggcggcctggaccccccatgggagacgctgagt

SEQ ID NO: 445 - DNA, 48 bp, Homo sapiens
  source 1..48: mol_type = genomic DNA
catggaaaataaagccaggaaagtcaaaaaacgaaagagaaggagaag

SEQ ID NO: 446 - DNA, 48 bp, Homo sapiens
  source 1..48: mol_type = genomic DNA
ctgtggggagggcggtggggggtgccagcctgccatgcgtgcaggggt

SEQ ID NO: 447 - DNA, 47 bp, Homo sapiens
  source 1..47: mol_type = genomic DNA
cttggcctttttttgatgtgctttagcaaaggttggactgaatgggg

SEQ ID NO: 448 - DNA, 46 bp, Homo sapiens
  source 1..46: mol_type = genomic DNA
ccatggccatgccagaggtaaaaaacgacggcggcggaagcagaag

SEQ ID NO: 449 - DNA, 47 bp, Homo sapiens
  source 1..47: mol_type = genomic DNA
catagagaggcggccaccactgccatcggagggggggtggcccgggt

SEQ ID NO: 450 - DNA, 50 bp, Homo sapiens
  source 1..50: mol_type = genomic DNA
ctggggggccccaataaattacattcttgagagagcatagtgtgtggggg

SEQ ID NO: 451 - DNA, 49 bp, Homo sapiens
  source 1..49: mol_type = genomic DNA
cctgggggggtcctggctggccgctccttcctcctggcttcccgagggt

SEQ ID NO: 452 - DNA, 48 bp, Homo sapiens
  source 1..48: mol_type = genomic DNA
ctgcgagctgtggtggtggatgactaccgtcggcgcaaaaaaagggag

SEQ ID NO: 453 - DNA, 47 bp, Homo sapiens
  source 1..47: mol_type = genomic DNA
ctaagaaaatgcccaaaaaataggcaaaacacgagaagagctagggt

SEQ ID NO: 454 - DNA, 46 bp, Homo sapiens
  source 1..46: mol_type = genomic DNA
cggagatcctctttaaaaaaaaatgcatatgcaatgtcccaagaat

SEQ ID NO: 455 - DNA, 50 bp, Homo sapiens
  source 1..50: mol_type = genomic DNA
ctcagccgaggcttaatggaagaactggttagcattttttttttttgagg

SEQ ID NO: 456 - DNA, 50 bp, Homo sapiens
  source 1..50: mol_type = genomic DNA
ctctgcccgggggtgtcaggcaccggatctcacgggagttcctcctggag

SEQ ID NO: 457 - DNA, 46 bp, Homo sapiens
  source 1..46: mol_type = genomic DNA
ccaggggggatggtggtgcactcggggaggcgggaagaggaagaag

SEQ ID NO: 458 - DNA, 49 bp, Homo sapiens
  source 1..49: mol_type = genomic DNA
ctggggggtatcacccagagggtggtggaaggcgtcaaagtgtagggag

SEQ ID NO: 459 - DNA, 47 bp, Homo sapiens
  source 1..47: mol_type = genomic DNA
ccccggggggcggctccgcgtggggttcggcgaccgtcaggtggaag

SEQ ID NO: 460 - DNA, 48 bp, Homo sapiens
  source 1..48: mol_type = genomic DNA
ctgggttctgacagagaagctggggattggcagggggtggcatcggag

SEQ ID NO: 461 - DNA, 50 bp, Homo sapiens
  source 1..50: mol_type = genomic DNA
cctcgggaccctaaaatccctaagagcaagcgccaaaaaaggaggtgagt

SEQ ID NO: 462 - DNA, 49 bp, Homo sapiens
  source 1..49: mol_type = genomic DNA
ccctgatgcgggagctggatgaggagggctctgatcccccctgccgggg

SEQ ID NO: 463 - DNA, 46 bp, Homo sapiens
  source 1..46: mol_type = genomic DNA
ctgggttgggaaacccattgctcgagtggttaaaaaaagaccggag

SEQ ID NO: 464 - DNA, 49 bp, Homo sapiens
  source 1..49: mol_type = genomic DNA
cggagggggggtggcccgggtggaggtggcggcggggccaccgatgagg

SEQ ID NO: 465 - DNA, 46 bp, Homo sapiens
  source 1..46: mol_type = genomic DNA
cagagtcatgggggcccccccaaggcgcggtgccccagagtggggt

SEQ ID NO: 466 - DNA, 49 bp, Homo sapiens
  source 1..49: mol_type = genomic DNA
ctgtgacccccccttcctccctggtgaggtcggagccagagggctgggg

SEQ ID NO: 467 - DNA, 50 bp, Homo sapiens
  source 1..50: mol_type = genomic DNA
ccctgggaggtggcttacatggtggtgagcagaggggggtgtagtcgggg

SEQ ID NO: 468 - DNA, 46 bp, Homo sapiens
  source 1..46: mol_type = genomic DNA
ccctgcggaatttaaacctccaaaaaagcagctgctttcagaggag

SEQ ID NO: 469 - DNA, 46 bp, Homo sapiens
  source 1..46: mol_type = genomic DNA
ccaggaagaaggcatgggggggccacgatcatatagctctcggagg

SEQ ID NO: 470 - DNA, 50 bp, Homo sapiens
  source 1..50: mol_type = genomic DNA
catgggggggggtttaatttggtttctgagcgcataaagctaaggagggg

SEQ ID NO: 471 - DNA, 46 bp, Homo sapiens
  source 1..46: mol_type = genomic DNA
cgtagctcccagagctgtagggggggactaaaaggagggcaagagg

SEQ ID NO: 472 - DNA, 48 bp, Homo sapiens
  source 1..48: mol_type = genomic DNA
catgggggggggtttaatttggtttctgagcgcataaagctaaggagg

SEQ ID NO: 473 - DNA, 47 bp, Homo sapiens
  source 1..47: mol_type = genomic DNA
cggtgcctgcctgcatgtcccctcatgcccaccccctggctctgggg

SEQ ID NO: 474 - DNA, 46 bp, Homo sapiens
  source 1..46: mol_type = genomic DNA
caaagcatgcagagtgctatttctttttttttctcttgaccagaag

SEQ ID NO: 475 - DNA, 49 bp, Homo sapiens
  source 1..49: mol_type = genomic DNA
ccgtgcgagaccccgctaccacacggccgcctcgttcatttcggggggt

SEQ ID NO: 476 - DNA, 49 bp, Homo sapiens
  source 1..49: mol_type = genomic DNA
cctcgccgtatatccaacattaaaaagaaaaaaaaggctgtttaagaat

SEQ ID NO: 477 - DNA, 47 bp, Homo sapiens
  source 1..47: mol_type = genomic DNA
ctatgtcatcaataatcataaaacgtattttttttttgagtcagagt

SEQ ID NO: 478 - DNA, 50 bp, Homo sapiens
  source 1..50: mol_type = genomic DNA
ctggggggtatcacccagagggtggtggaaggcgtcaaagtgtagggagt

SEQ ID NO: 479 - DNA, 49 bp, Homo sapiens
  source 1..49: mol_type = genomic DNA
ccatgaccatgggcacagttcagagatgggaaaaaaagtgggtgagaag

SEQ ID NO: 480 - DNA, 50 bp, Homo sapiens
  source 1..50: mol_type = genomic DNA
cagggggctctgctctcccttgcctgacaggagacgcactcggcccgagt

SEQ ID NO: 481 - DNA, 48 bp, Homo sapiens
  source 1..48: mol_type = genomic DNA
cagagtgagactccctctcaaaaaaaaaaaaagagagagagcgagaat

SEQ ID NO: 482 - DNA, 46 bp, Homo sapiens
  source 1..46: mol_type = genomic DNA
cagagtgccattttttttttgttcaaatgattttaattattggaat

SEQ ID NO: 483 - DNA, 48 bp, Homo sapiens
  source 1..48: mol_type = genomic DNA
cagggaaaaaaaaaatatatatatatataaatacccctacatttgaag

SEQ ID NO: 484 - DNA, 49 bp, Homo sapiens
  source 1..49: mol_type = genomic DNA
ccaagtctttttttcgggacccacgagacgtgagtggaggccaaagggg

SEQ ID NO: 485 - DNA, 48 bp, Homo sapiens
  source 1..48: mol_type = genomic DNA
ctcaggcacgaggatggcgatgagaccacggagccacccccagtgggt

SEQ ID NO: 486 - DNA, 47 bp, Homo sapiens
  source 1..47: mol_type = genomic DNA
ccaagaacatgactatttcaaggggggactgatgcagtgtgaggaat

SEQ ID NO: 487 - DNA, 49 bp, Homo sapiens
  source 1..49: mol_type = genomic DNA
cctcgggaagccaggaggaaggagcggccagccaggacccccccaggag

SEQ ID NO: 488 - DNA, 49 bp, Homo sapiens
  source 1..49: mol_type = genomic DNA
cctggcccgggagtcattggggggatcatgacagagaagcagggggggt

SEQ ID NO: 489 - DNA, 49 bp, Homo sapiens
  source 1..49: mol_type = genomic DNA
caccgccaccaagaaagcagtccgatgagattttttttggaggggggag

SEQ ID NO: 490 - DNA, 47 bp, Homo sapiens
  source 1..47: mol_type = genomic DNA
ccctgccgagcctggatatcgtagtgtggtcggagctgcccccgggg

SEQ ID NO: 491 - DNA, 49 bp, Homo sapiens
  source 1..49: mol_type = genomic DNA
cacagcctcttcctctttttttcccctcctagccctattcaggcaggag

SEQ ID NO: 492 - DNA, 50 bp, Homo sapiens
  source 1..50: mol_type = genomic DNA
ctgtgacccccccttcctccctggtgaggtcggagccagagggctggggg

SEQ ID NO: 493 - DNA, 49 bp, Homo sapiens
  source 1..49: mol_type = genomic DNA
ctccgtggccttagctgtgctcgcgctatctctctttctggcctggagg

SEQ ID NO: 494 - DNA, 46 bp, Homo sapiens
  source 1..46: mol_type = genomic DNA
cctcggtcctaccccctgacctgcgctgcaactacagcatccgggt

SEQ ID NO: 495 - DNA, 48 bp, Homo sapiens
  source 1..48: mol_type = genomic DNA
cagagtgagaccttattgctaaaaaaaaataaaaataaaccaagggat

SEQ ID NO: 496 - DNA, 46 bp, Homo sapiens
  source 1..46: mol_type = genomic DNA
caaaggcacaaagtttaaacatgggggggcgggtgttgagaggggt

SEQ ID NO: 497 - DNA, 47 bp, Homo sapiens
  source 1..47: mol_type = genomic DNA
ccaggtggccttctctctgcacccccccaccaaccccagggatgggg

SEQ ID NO: 498 - DNA, 50 bp, Homo sapiens
  source 1..50: mol_type = genomic DNA
caaggaggagatgaaaaaaacaacatcccagagccagttgtcatcggaat

SEQ ID NO: 499 - DNA, 46 bp, Homo sapiens
  source 1..46: mol_type = genomic DNA
ctaggaaggacaaggccgaattaaaaaaaaatcttcattaaagaat

SEQ ID NO: 500 - DNA, 48 bp, Homo sapiens
  source 1..48: mol_type = genomic DNA
ccaagggccagggagcccaggggtggctacagtggagagggcttgggg

SEQ ID NO: 501 - DNA, 47 bp, Homo sapiens
  source 1..47: mol_type = genomic DNA
catgggggggggtttaatttggtttctgagcgcataaagctaaggag

SEQ ID NO: 502 - DNA, 50 bp, Homo sapiens
  source 1..50: mol_type = genomic DNA
ccctgcttgtggtgcagggggtgccattgccgtggtgcttggcattgagg

SEQ ID NO: 503 - DNA, 47 bp, Homo sapiens
  source 1..47: mol_type = genomic DNA
cacagcatcaaaaaaggagcctgagatctcagatacgtgtacagagt

SEQ ID NO: 504 - DNA, 46 bp, Homo sapiens
  source 1..46: mol_type = genomic DNA
ctggggcaccgcgccttgggggggcccccatgactctggggtgggt

SEQ ID NO: 505 - DNA, 47 bp, Homo sapiens
  source 1..47: mol_type = genomic DNA
cctgggggggtcctggctggccgctccttcctcctggcttcccgagg

SEQ ID NO: 506 - DNA, 48 bp, Homo sapiens
  source 1..48: mol_type = genomic DNA
ctccgtggccttagctgtgctcgcgctatctctctttctggcctggag

SEQ ID NO: 507 - DNA, 49 bp, Homo sapiens
  source 1..49: mol_type = genomic DNA
cgtagggaggggggaacggaaatagtgatcctcccccaccgaagagggg

SEQ ID NO: 508 - DNA, 49 bp, Homo sapiens
  source 1..49: mol_type = genomic DNA
ctctgcatctacagcaggagagggtgcctgaggtgtggggggatggggg

SEQ ID NO: 509 - DNA, 50 bp, Homo sapiens
  source 1..50: mol_type = genomic DNA
cctcgggaagccaggaggaaggagcggccagccaggacccccccaggagg

SEQ ID NO: 510 - DNA, 47 bp, Homo sapiens
  source 1..47: mol_type = genomic DNA
caagggccagggagcccaggggtggctacagtggagagggcttgggg

SEQ ID NO: 511 - DNA, 48 bp, Homo sapiens
  source 1..48: mol_type = genomic DNA
cctggcccgggagtcattggggggatcatgacagagaagcaggggggg

SEQ ID NO: 512 - DNA, 48 bp, Homo sapiens
  source 1..48: mol_type = genomic DNA
ccaagaaaaaaaatcaatcagaataaactcaaaaaaaaaggtaggggg

SEQ ID NO: 513 - DNA, 49 bp, Homo sapiens
  source 1..49: mol_type = genomic DNA
ctttgtgataaggggttattttatgctaattcacaagttttttttgaag

SEQ ID NO: 514 - DNA, 50 bp, Homo sapiens
  source 1..50: mol_type = genomic DNA
ccaagaaaaaaaatcaatcagaataaactcaaaaaaaaaggtagggggag

SEQ ID NO: 515 - DNA, 48 bp, Homo sapiens
  source 1..48: mol_type = genomic DNA
cttagggaaagatatggtgaaaaaaaagaaatgctactcggtaggaag

SEQ ID NO: 516 - DNA, 46 bp, Homo sapiens
  source 1..46: mol_type = genomic DNA
cccaggctctctgttaaccagcttatgtccagcagagctggggggt

SEQ ID NO: 517 - DNA, 47 bp, Homo sapiens
  source 1..47: mol_type = genomic DNA
ccaagaaaaaaaatcaatcagaataaactcaaaaaaaaaggtagggg

SEQ ID NO: 518 - DNA, 47 bp, Homo sapiens
  source 1..47: mol_type = genomic DNA
cccggatgctgtagttgcagcgcaggtcagggggtaggaccgagggt

SEQ ID NO: 519 - DNA, 46 bp, Homo sapiens
  source 1..46: mol_type = genomic DNA
cgtagcacgtggcgctgatgcccgagttactgctggggggcagggg

SEQ ID NO: 520 - DNA, 50 bp, Homo sapiens
  source 1..50: mol_type = genomic DNA
ccaagtctttttttcgggacccacgagacgtgagtggaggccaaaggggt

SEQ ID NO: 521 - DNA, 50 bp, Homo sapiens
  source 1..50: mol_type = genomic DNA
caccgccaccaagaaagcagtccgatgagattttttttggaggggggagg

SEQ ID NO: 522 - DNA, 47 bp, Homo sapiens
  source 1..47: mol_type = genomic DNA
cagcggcaggggcggagccccgggggcggcactataataataagggg

SEQ ID NO: 523 - DNA, 49 bp, Homo sapiens
  source 1..49: mol_type = genomic DNA
cagtggggctggaagcagaaacaaaatgaaaaaaaaggggggtgggagg

SEQ ID NO: 524 - DNA, 50 bp, Homo sapiens
  source 1..50: mol_type = genomic DNA
cctggaggcttggacgacagatccccccaggctcctctgagactgtggag

SEQ ID NO: 525 - DNA, 50 bp, Homo sapiens
  source 1..50: mol_type = genomic DNA
ccagggggtgggcatgaggggacatgcaggcaggcaccgggtcgcagggg

SEQ ID NO: 526 - DNA, 47 bp, Homo sapiens
  source 1..47: mol_type = genomic DNA
caccgccaccaagaaagcagtccgatgagattttttttggagggggg

SEQ ID NO: 527 - DNA, 47 bp, Homo sapiens
  source 1..47: mol_type = genomic DNA
ctgggcccatggaacggtggggggtgcgcttgattctacttcaggag

SEQ ID NO: 528 - DNA, 46 bp, Homo sapiens
  source 1..46: mol_type = genomic DNA
caaagtcaacagatagtgccaaaagacccttaaaaaaaaacaggat

SEQ ID NO: 529 - DNA, 46 bp, Homo sapiens; identical to SEQ ID NO: 528
  source 1..46: mol_type = genomic DNA

SEQ ID NO: 530 - DNA, 50 bp, Homo sapiens
  source 1..50: mol_type = genomic DNA
ctctgacatggtttttttttcttttttgaggggcattttaaacttagagg

SEQ ID NO: 531 - DNA, 49 bp, Homo sapiens
  source 1..49: mol_type = genomic DNA
ccctgaactaggagccaccatgttggtgatacccccggactgagcgagg

SEQ ID NO: 532 - DNA, 48 bp, Homo sapiens
  source 1..48: mol_type = genomic DNA
cccaggggtggctacagtggagagggcttggggcgtactccggtgagt

SEQ ID NO: 533 - DNA, 48 bp, Homo sapiens
  source 1..48: mol_type = genomic DNA
ctgggcccatggaacggtggggggtgcgcttgattctacttcaggagg

SEQ ID NO: 534 - DNA, 46 bp, Homo sapiens
  source 1..46: mol_type = genomic DNA
ccccgctcctggcccgggagtcattggggggatcatgacagagaag

SEQ ID NO: 535 - DNA, 48 bp, Homo sapiens
  source 1..48: mol_type = genomic DNA
caaagaaggaagaggaggaaaaggaaaaaaaaggggtatattgtggat

SEQ ID NO: 536 - DNA, 50 bp, Homo sapiens
  source 1..50: mol_type = genomic DNA
cgtagggaggggggaacggaaatagtgatcctcccccaccgaagaggggg

SEQ ID NO: 537 - DNA, 47 bp, Homo sapiens
  source 1..47: mol_type = genomic DNA
cgtagggaggggggaacggaaatagtgatcctcccccaccgaagagg

SEQ ID NO: 538 - DNA, 46 bp, Homo sapiens
  source 1..46: mol_type = genomic DNA
cctggggggccagcgcgggcacctgggggtgtgcctgcaggggggt

SEQ ID NO: 539 - DNA, 48 bp, Homo sapiens
  source 1..48: mol_type = genomic DNA
ctgggggggtcctggctggccgctccttcctcctggcttcccgagggt

SEQ ID NO: 540 - DNA, 46 bp, Homo sapiens
  source 1..46: mol_type = genomic DNA
ccaagaaaaaaaaaaagaaaaaaaaaacaacatggctgcaaaggag

SEQ ID NO: 541 - DNA, 48 bp, Homo sapiens
  source 1..48: mol_type = genomic DNA
cactgcccatatctgtggactgccccttccaaagacccctgggggggt

SEQ ID NO: 542 - DNA, 48 bp, Homo sapiens
  source 1..48: mol_type = genomic DNA
ccgagggcagtccccggggggctgcagctccagggggcctgggaggag

SEQ ID NO: 543 - DNA, 47 bp, Homo sapiens
  source 1..47: mol_type = genomic DNA
cctggcccgggagtcattggggggatcatgacagagaagcagggggg

SEQ ID NO: 544 - DNA, 49 bp, Homo sapiens
  source 1..49: mol_type = genomic DNA
cgcagccagagggccagggggtcccacatctggccgaagggcttcgagg

SEQ ID NO: 545 - DNA, 49 bp, Homo sapiens
  source 1..49: mol_type = genomic DNA
cagagtgccaagggcccagacaccatgtgagcagcagccagcggggggg

SEQ ID NO: 546 - DNA, 46 bp, Homo sapiens
  source 1..46: mol_type = genomic DNA
cccgggggtgtcaggcaccggatctcacgggagttcctcctggagg

SEQ ID NO: 547 - DNA, 48 bp, Homo sapiens
  source 1..48: mol_type = genomic DNA
ctctgcatctacagcaggagagggtgcctgaggtgtggggggatgggg

SEQ ID NO: 548 - DNA, 46 bp, Homo sapiens
  source 1..46: mol_type = genomic DNA
caccgccaccaagaaagcagtccgatgagattttttttggaggggg

SEQ ID NO: 549 - DNA, 46 bp, Homo sapiens
  source 1..46: mol_type = genomic DNA
caggggggcagtgctggggggtacagggaactggactgtctcggat

SEQ ID NO: 550 - DNA, 48 bp, Homo sapiens
  source 1..48: mol_type = genomic DNA
cagtggggctggaagcagaaacaaaatgaaaaaaaaggggggtgggag

SEQ ID NO: 551 - DNA, 49 bp, Homo sapiens
  source 1..49: mol_type = genomic DNA
ctcggtcctaccccctgacctgcgctgcaactacagcatccgggtggag

SEQ ID NO: 552 - DNA, 50 bp, Homo sapiens
  source 1..50: mol_type = genomic DNA
ctctgcatctacagcaggagagggtgcctgaggtgtggggggatgggggt

SEQ ID NO: 553 - DNA, 49 bp, Homo sapiens
  source 1..49: mol_type = genomic DNA
cgggggcggcactataataataaggggaacctggaagttaacacaggag

SEQ ID NO: 554 - DNA, 46 bp, Homo sapiens
  source 1..46: mol_type = genomic DNA
ccctgacctctttttttttttccgctctgtcctgggtgaccagggt

SEQ ID NO: 555 - DNA, 49 bp, Homo sapiens
  source 1..49: mol_type = genomic DNA
catggagggcgcttcccagtactaagctattaccacaaaaaaatgggat

SEQ ID NO: 556 - DNA, 46 bp, Homo sapiens
  source 1..46: mol_type = genomic DNA
cctggcccgggagtcattggggggatcatgacagagaagcaggggg

SEQ ID NO: 557 - DNA, 46 bp, Homo sapiens
  source 1..46: mol_type = genomic DNA
ccgagggcagtccccggggggctgcagctccagggggcctgggagg

SEQ ID NO: 558 - DNA, 50 bp, Homo sapiens
  source 1..50: mol_type = genomic DNA
cactgacagggtctgtactttttttttctttttgagtcaggactatggag

SEQ ID NO: 559 - DNA, 47 bp, Homo sapiens
  source 1..47: mol_type = genomic DNA
ctcagactttttttttttaatgggatttttaggtcagcccaggggag

SEQ ID NO: 560 - DNA, 49 bp, Homo sapiens
  source 1..49: mol_type = genomic DNA
ctcagcctccataaatctaatgtcatttttttttaataagtcctaggag

SEQ ID NO: 561 - DNA, 46 bp, Homo sapiens
  source 1..46: mol_type = genomic DNA
caaggaataaagttaaaaaaaaaaaagaaaaagaaaaaaggtgagt

SEQ ID NO: 562 - DNA, 50 bp, Homo sapiens
  source 1..50: mol_type = genomic DNA
cagggcctcgggctcccagtacagtgccccctgcttgtggtgcagggggt

SEQ ID NO: 563 - DNA, 47 bp, Homo sapiens
  source 1..47: mol_type = genomic DNA
cagtgccagccaccgggtgtgtgtgcctgcgagccgggctggggggt

SEQ ID NO: 564 - DNA, 46 bp, Homo sapiens
  source 1..46: mol_type = genomic DNA
catggagacgccgggggactggccaagggccagggagcccaggggt

SEQ ID NO: 565 - DNA, 48 bp, Homo sapiens
  source 1..48: mol_type = genomic DNA
ccaggggaaaaaaatttgttttggtgttttttgagatattctttggat

SEQ ID NO: 566 - DNA, 48 bp, Homo sapiens
  source 1..48: mol_type = genomic DNA
ctgggtgggcggggggaggacacgccttactctaactggcacaaggag

SEQ ID NO: 567 - DNA, 46 bp, Homo sapiens
  source 1..46: mol_type = genomic DNA
ctgggggggtcctggctggccgctccttcctcctggcttcccgagg

SEQ ID NO: 568 - DNA, 50 bp, Homo sapiens
  source 1..50: mol_type = genomic DNA
cattggccaaagtgaaaattttttttttcttttgaaatctagttttgaat

SEQ ID NO: 569 - DNA, 46 bp, Homo sapiens
  source 1..46: mol_type = genomic DNA
ctgtgtgtcactagagaaaaaaaaaacaaaaacctagattccggat

SEQ ID NO: 570 - DNA, 48 bp, Homo sapiens
  source 1..48: mol_type = genomic DNA
ccgagtgcaccaccatcccccctggaaacactgcaggaaacagggggg

SEQ ID NO: 571 - DNA, 49 bp, Homo sapiens
  source 1..49: mol_type = genomic DNA
cgctgccagggctctgcccgggggtgtcaggcaccggatctcacgggag

SEQ ID NO: 572 - DNA, 46 bp, Homo sapiens
  source 1..46: mol_type = genomic DNA
catggaaccaagtggattttttggcactgtttattctttgcagaag

SEQ ID NO: 573 - DNA, 47 bp, Homo sapiens
  source 1..47: mol_type = genomic DNA
ccgtggccttagctgtgctcgcgctatctctctttctggcctggagg

SEQ ID NO: 574 - DNA, 48 bp, Homo sapiens
  source 1..48: mol_type = genomic DNA
cagagtgccaagggcccagacaccatgtgagcagcagccagcgggggg

SEQ ID NO: 575 - DNA, 50 bp, Homo sapiens
  source 1..50: mol_type = genomic DNA
cctgggcgacacaccaaggctctgtctcaaaaaaaaaaaatttagagagg

SEQ ID NO: 576 - DNA, 49 bp, Homo sapiens
  source 1..49: mol_type = genomic DNA
ctaagagcgggtcaagaaattgaaaaaaaaaacaaaacatttaaggggt

SEQ ID NO: 577 - DNA, 50 bp, Homo sapiens
  source 1..50: mol_type = genomic DNA
cagagtgccaagggcccagacaccatgtgagcagcagccagcgggggggg

SEQ ID NO: 578 - DNA, 46 bp, Homo sapiens
  source 1..46: mol_type = genomic DNA
ccgtggccttagctgtgctcgcgctatctctctttctggcctggag

SEQ ID NO: 579 - DNA, 47 bp, Homo sapiens
  source 1..47: mol_type = genomic DNA
cgagggcagtccccggggggctgcagctccagggggcctgggaggag

SEQ ID NO: 580 - DNA, 47 bp, Homo sapiens
  source 1..47: mol_type = genomic DNA
cagagtgccaagggcccagacaccatgtgagcagcagccagcggggg

SEQ ID NO: 581 - DNA, 46 bp, Homo sapiens
  source 1..46: mol_type = genomic DNA
ctctgcctcttgccctccttttagtccccccctacagctctgggag

SEQ ID NO: 582 - DNA, 48 bp, Homo sapiens
  source 1..48: mol_type = genomic DNA
ccttgcgcagggtccgggcagggagggctggggggtatcacccagagg

SEQ ID NO: 583 - DNA, 46 bp, Homo sapiens
  source 1..46: mol_type = genomic DNA
cctggggggatctgtcgtccaagcctccaggcctctcggcaggggt

SEQ ID NO: 584 - DNA, 46 bp, Homo sapiens
  source 1..46: mol_type = genomic DNA
cagggaaaggagcccccctgtttcctgcagtgtttccaggggggat

SEQ ID NO: 585 - DNA, 49 bp, Homo sapiens
  source 1..49: mol_type = genomic DNA
cactgtctccaaaaaaaaatgtttaaaatgagaccaaaccctcatggag

SEQ ID NO: 586 - DNA, 46 bp, Homo sapiens
  source 1..46: mol_type = genomic DNA
cactgccatcggagggggggtggcccgggtggaggtggcggcgggg

SEQ ID NO: 587 - DNA, 50 bp, Homo sapiens
  source 1..50: mol_type = genomic DNA
cacagcctccccccatgagcttggggctggcgggggcacaggaggtggag

SEQ ID NO: 588 - DNA, 50 bp, Homo sapiens
  source 1..50: mol_type = genomic DNA
cacggggagcggaaggagttcgtgtgccactggggggctgctccagggag

SEQ ID NO: 589 - DNA, 50 bp, Homo sapiens
  source 1..50: mol_type = genomic DNA
ccttgcgcagggtccgggcagggagggctggggggtatcacccagagggt

SEQ ID NO: 590 - DNA, 50 bp, Homo sapiens
  source 1..50: mol_type = genomic DNA
cgctgccagggctctgcccgggggtgtcaggcaccggatctcacgggagt

SEQ ID NO: 591 - DNA, 50 bp, Homo sapiens
  source 1..50: mol_type = genomic DNA
ccgggggcggcactataataataaggggaacctggaagttaacacaggag

SEQ ID NO: 592 - DNA, 47 bp, Homo sapiens
  source 1..47: mol_type = genomic DNA
ccgagtgcaccaccatcccccctggaaacactgcaggaaacaggggg

SEQ ID NO: 593 - DNA, 46 bp, Homo sapiens
  source 1..46: mol_type = genomic DNA
ccctgacgccccatccctggggttggtgggggggtgcagagagaag

SEQ ID NO: 594 - DNA, 47 bp, Homo sapiens
  source 1..47: mol_type = genomic DNA
ccaggggtggctacagtggagagggcttggggcgtactccggtgagt

SEQ ID NO: 595 - DNA, 48 bp, Homo sapiens
  source 1..48: mol_type = genomic DNA
cagagagcacagcacctcatcatgatttttttacacagtctcaggaat

SEQ ID NO: 596 - DNA, 47 bp, Homo sapiens
  source 1..47: mol_type = genomic DNA
ctttgacttcatttttttccacacatccccactgtgccagagggaat

SEQ ID NO: 597 - DNA, 49 bp, Homo sapiens
  source 1..49: mol_type = genomic DNA
cgaagggccgggctgcggcgggggctgctggtggtggtggtgggggggt

SEQ ID NO: 598 - DNA, 46 bp, Homo sapiens
  source 1..46: mol_type = genomic DNA
ccttgagggctggcgtgccggggggtagctgccatacaggtggaag

SEQ ID NO: 599 - DNA, 48 bp, Homo sapiens
  source 1..48: mol_type = genomic DNA
cgatgagaccacggagccacccccagtgggtgccgggaccggaggagg

SEQ ID NO: 600 - DNA, 49 bp, Homo sapiens
  source 1..49: mol_type = genomic DNA
cagggggtgggcatgaggggacatgcaggcaggcaccgggtcgcagggg

SEQ ID NO: 601 - DNA, 46 bp, Homo sapiens
  source 1..46: mol_type = genomic DNA
cctgggctgacctaaaaatcccattaaaaaaaaaaagtctgagagt

SEQ ID NO: 602 - DNA, 48 bp, Homo sapiens
  source 1..48: mol_type = genomic DNA
ctaagagcgggtcaagaaattgaaaaaaaaaacaaaacatttaagggg

SEQ ID NO: 603 - DNA, 47 bp, Homo sapiens
  source 1..47: mol_type = genomic DNA
ccgaggcttaatggaagaactggttagcattttttttttttgagggt

SEQ ID NO: 604 - DNA, 46 bp, Homo sapiens
  source 1..46: mol_type = genomic DNA
cacagagaaataaaaaggaacaaaaatcacattctaatgggggggt

SEQ ID NO: 605 - DNA, 46 bp, Homo sapiens
  source 1..46: mol_type = genomic DNA
ctaagagatggtcaaaggcacaaagtttaaacatgggggggcgggt

SEQ ID NO: 606 - DNA, 48 bp, Homo sapiens
  source 1..48: mol_type = genomic DNA
ccctgtcttgattttagcatttttttcccagtgttaggtgaaaaggat

SEQ ID NO: 607 - DNA, 46 bp, Homo sapiens
  source 1..46: mol_type = genomic DNA
ccaggccagaaagagagatagcgcgagcacagctaaggccacggag

SEQ ID NO: 608 - DNA, 47 bp, Homo sapiens
  source 1..47: mol_type = genomic DNA
ctggggctggaggggggatctcggagccaggcatgtcaccattgggt

SEQ ID NO: 609 - DNA, 46 bp, Homo sapiens
  source 1..46: mol_type = genomic DNA
cggagggaccccccgccttttcctctgtgggtgtcgggcagagagg

SEQ ID NO: 610 - DNA, 46 bp, Homo sapiens
  source 1..46: mol_type = genomic DNA
cggggatggaagtccaggcgggggttgcactggagcgtcaaaggag

SEQ ID NO: 611 - DNA, 49 bp, Homo sapiens
  source 1..49: mol_type = genomic DNA
ctgggcgacacaccaaggctctgtctcaaaaaaaaaaaatttagagagg

SEQ ID NO: 612 - DNA, 46 bp, Homo sapiens
  source 1..46: mol_type = genomic DNA
ccttgactccagccaaggacaagaaaaagaaagacaaaaaaagaag

SEQ ID NO: 613 - DNA, 50 bp, Homo sapiens
  source 1..50: mol_type = genomic DNA
ccttgactccagccaaggacaagaaaaagaaagacaaaaaaagaaggaat

SEQ ID NO: 614 - DNA, 47 bp, Homo sapiens
  source 1..47: mol_type = genomic DNA
cctggagacaatccactgctgtcaaacacttcatctggtgggggggt

SEQ ID NO: 615 - DNA, 50 bp, Homo sapiens
  source 1..50: mol_type = genomic DNA
ccaagactgcacccccccttgaagagctgtgagtcctgacggggaagggg

SEQ ID NO: 616 - DNA, 48 bp, Homo sapiens
  source 1..48: mol_type = genomic DNA
cggggagcggaaggagttcgtgtgccactggggggctgctccagggag

SEQ ID NO: 617 - DNA, 47 bp, Homo sapiens
  source 1..47: mol_type = genomic DNA
ccaagactgcacccccccttgaagagctgtgagtcctgacggggaag

SEQ ID NO: 618 - DNA, 47 bp, Homo sapiens
  source 1..47: mol_type = genomic DNA
ccttgagtgcagctcctccaccgcgttcaccctgcattttttagagg

SEQ ID NO: 619 - DNA, 48 bp, Homo sapiens
  source 1..48: mol_type = genomic DNA
ccccgagtgcaccaccatcccccctggaaacactgcaggaaacagggg

SEQ ID NO: 620 - DNA, 50 bp, Homo sapiens
  source 1..50: mol_type = genomic DNA
ccccgagtgcaccaccatcccccctggaaacactgcaggaaacagggggg

SEQ ID NO: 621 - DNA, 49 bp, Homo sapiens
  source 1..49: mol_type = genomic DNA
ccccgagtgcaccaccatcccccctggaaacactgcaggaaacaggggg

SEQ ID NO: 622 - DNA, 46 bp, Homo sapiens
  source 1..46: mol_type = genomic DNA
ccaagggcccagacaccatgtgagcagcagccagcggggggggggg

SEQ ID NO: 623 - DNA, 48 bp, Homo sapiens
  source 1..48: mol_type = genomic DNA
ctaggacacaggtgggtttttttgtttttttgtttttttttgatggag

SEQ ID NO: 624 - DNA, 47 bp, Homo sapiens
  source 1..47: mol_type = genomic DNA
cagtgactgtggaaaggctgctggctgtggggagggcggtggggggt

SEQ ID NO: 625 - DNA, 47 bp, Homo sapiens
  source 1..47: mol_type = genomic DNA
caaagacttgagagatgcttttttttcccccagtgaggggactggag

SEQ ID NO: 626 - DNA, 48 bp, Homo sapiens
  source 1..48: mol_type = genomic DNA
caaagacttgagagatgcttttttttcccccagtgaggggactggagg

SEQ ID NO: 627 - DNA, 46 bp, Homo sapiens
  source 1..46: mol_type = genomic DNA
cagggggccaaaggagacacccccaagggcctccgggatggcgagt

SEQ ID NO: 628 - DNA, 50 bp, Homo sapiens
  source 1..50: mol_type = genomic DNA
cgaggtgcccattggctgactgttcatgtgtgtctgcatatgtggggggt

SEQ ID NO: 629 - DNA, 47 bp, Homo sapiens
  source 1..47: mol_type = genomic DNA
cccagggactacctcggcttttaatttaaaaaaaaaaagaagtgggt

SEQ ID NO: 630 - DNA, 49 bp, Homo sapiens
  source 1..49: mol_type = genomic DNA
cccagagccagggggtgggcatgaggggacatgcaggcaggcaccgggt

SEQ ID NO: 631 - DNA, 50 bp, Homo sapiens
  source 1..50: mol_type = genomic DNA
caaagacttgagagatgcttttttttcccccagtgaggggactggaggat

SEQ ID NO: 632 - DNA, 49 bp, Homo sapiens
  source 1..49: mol_type = genomic DNA
ctaggacacaggtgggtttttttgtttttttgtttttttttgatggagt

SEQ ID NO: 633 - DNA, 49 bp, Homo sapiens
  source 1..49: mol_type = genomic DNA
ccgggggactggccaagggccagggagcccaggggtggctacagtggag

SEQ ID NO: 634 - DNA, 47 bp, Homo sapiens
  source 1..47: mol_type = genomic DNA
cccagagtcatgggggcccccccaaggcgcggtgccccagagtgggg

SEQ ID NO: 635 - DNA, 47 bp, Homo sapiens
  source 1..47: mol_type = genomic DNA
cctggagcatcccccgccgcagcagagccgagtgtggaagtacgagg

SEQ ID NO: 636 - DNA, 46 bp, Homo sapiens
  source 1..46: mol_type = genomic DNA
cgtggccttagctgtgctcgcgctatctctctttctggcctggagg

SEQ ID NO: 637 - DNA, 48 bp, Homo sapiens
  source 1..48: mol_type = genomic DNA
ctcggagggaccccccgccttttcctctgtgggtgtcgggcagagagg

SEQ ID NO: 638 - DNA, 47 bp, Homo sapiens
  source 1..47: mol_type = genomic DNA
ctccgagagggcctgtggttggtggtggggggtgtcttctgcagaag

SEQ ID NO: 639 - DNA, 47 bp, Homo sapiens
  source 1..47: mol_type = genomic DNA
ccgcgacagggaagggagcacgttgatggggggtagatctgagggag

SEQ ID NO: 640 - DNA, 48 bp, Homo sapiens
  source 1..48: mol_type = genomic DNA
cagtgacttaacaatatacattcctcataaataaaaaaaaacaagaat

SEQ ID NO: 641 - DNA, 48 bp, Homo sapiens
  source 1..48: mol_type = genomic DNA
cccagagtcatgggggcccccccaaggcgcggtgccccagagtggggt

SEQ ID NO: 642 - DNA, 48 bp, Homo sapiens
  source 1..48: mol_type = genomic DNA
cagcgagccaaaaaatggaaccttcgacgaaaccgaccacttctggat

SEQ ID NO: 643 - DNA, 46 bp, Homo sapiens
  source 1..46: mol_type = genomic DNA
ccacgccttaaaaattgacagttgaaaaaaaaagagtgaccagagg

SEQ ID NO: 644 - DNA, 48 bp, Homo sapiens
  source 1..48: mol_type = genomic DNA
ctctgcctcaaaaaaaaagtatagaaaaatgagtagaaagcattgaat

SEQ ID NO: 645 - DNA, 49 bp, Homo sapiens
  source 1..49: mol_type = genomic DNA
cccggtgcctgcctgcatgtcccctcatgcccaccccctggctctgggg

SEQ ID NO: 646 - DNA, 48 bp, Homo sapiens
  source 1..48: mol_type = genomic DNA
ccctgtgtgtcactagagaaaaaaaaaacaaaaacctagattccggat

SEQ ID NO: 647 - DNA, 50 bp, Homo sapiens
  source 1..50: mol_type = genomic DNA
cagtgggtgaatctgcgccgggggtacccccgcctgaagaccttcggagg

SEQ ID NO: 648 - DNA, 50 bp, Homo sapiens
  source 1..50: mol_type = genomic DNA
ccgcgacagggaagggagcacgttgatggggggtagatctgagggagaag

SEQ ID NO: 649 - DNA, 47 bp, Homo sapiens
  source 1..47: mol_type = genomic DNA
ccgtgacctcaaactctttggactgtttgaaaaaaaaaaattggaag

SEQ ID NO: 650 - DNA, 47 bp, Homo sapiens
  source 1..47: mol_type = genomic DNA
ccctgggctgacctaaaaatcccattaaaaaaaaaaagtctgagagt

SEQ ID NO: 651 - DNA, 50 bp, Homo sapiens
  source 1..50: mol_type = genomic DNA
ccaggggagggcagggggccaaaggagacacccccaagggcctccgggat

SEQ ID NO: 652 - DNA, 46 bp, Homo sapiens
  source 1..46: mol_type = genomic DNA
cctggacttttcaagcattttttttgacaattaaattgggttggat

SEQ ID NO: 653 - DNA, 50 bp, Homo sapiens
  source 1..50: mol_type = genomic DNA
cttagcgtctcctggagcatcccccgccgcagcagagccgagtgtggaag

SEQ ID NO: 654 - DNA, 46 bp, Homo sapiens
  source 1..46: mol_type = genomic DNA
cctgggggcccgccaagactgcacccccccttgaagagctgtgagt

SEQ ID NO: 655 - DNA, 50 bp, Homo sapiens
  source 1..50: mol_type = genomic DNA
cagggtctgtactttttttttctttttgagtcaggactatggagccgagt

SEQ ID NO: 656 - DNA, 49 bp, Homo sapiens
  source 1..49: mol_type = genomic DNA
catggtgttgagagaaaaaaaaaaatcttttaaaagctgccatctgagg

SEQ ID NO: 657 - DNA, 50 bp, Homo sapiens
  source 1..50: mol_type = genomic DNA
cctggagcagccccccagtggcacacgaactccttccgctccccgtggat

SEQ ID NO: 658 - DNA, 48 bp, Homo sapiens
  source 1..48: mol_type = genomic DNA
caaagtgcttagacattttcaatttttttttgctaaatactttggaat

SEQ ID NO: 659 - DNA, 48 bp, Homo sapiens
  source 1..48: mol_type = genomic DNA
ccttgagaaagacaggaggtttcctgaatacaccgacacctggggggt

SEQ ID NO: 660 - DNA, 47 bp, Homo sapiens
  source 1..47: mol_type = genomic DNA
ccctgtctctgtctgtgatttttttttttctcggtggctctcgggat

SEQ ID NO: 661 - DNA, 50 bp, Homo sapiens
  source 1..50: mol_type = genomic DNA
ctaggagccaccatgttggtgatacccccggactgagcgaggaagaggag

SEQ ID NO: 662 - DNA, 48 bp, Homo sapiens
  source 1..48: mol_type = genomic DNA
ctaggagccaccatgttggtgatacccccggactgagcgaggaagagg

SEQ ID NO: 663 - DNA, 46 bp, Homo sapiens
  source 1..46: mol_type = genomic DNA
cccggggggcggctccgcgtggggttcggcgaccgtcaggtggaag

SEQ ID NO: 664 - DNA, 47 bp, Homo sapiens
  source 1..47: mol_type = genomic DNA
cccagagctgtagggggggactaaaaggagggcaagaggcagagggt

SEQ ID NO: 665 - DNA, 47 bp, Homo sapiens; identical to SEQ ID NO: 660
  source 1..47: mol_type = genomic DNA

SEQ ID NO: 666 - DNA, 48 bp, Homo sapiens
  source 1..48: mol_type = genomic DNA
cgaaggccgaaaaaaaaagacattgctgagtccattctggaaaagaat

SEQ ID NO: 667 - DNA, 49 bp, Homo sapiens
  source 1..49: mol_type = genomic DNA
cagtgggtgaatctgcgccgggggtacccccgcctgaagaccttcggag

SEQ ID NO: 668 - DNA, 47 bp, Homo sapiens
  source 1..47: mol_type = genomic DNA
caaagaggccacttttggaaaataatactttttttttttagttgaat

SEQ ID NO: 669 - DNA, 46 bp, Homo sapiens
  source 1..46: mol_type = genomic DNA
cttggaggagaaggtgccaaagcctgggcaggggtggctcctgggg

SEQ ID NO: 670 - DNA, 47 bp, Homo sapiens
  source 1..47: mol_type = genomic DNA
cagtgtcttcaggggtaggaggggaaaaaacggaaataactaggaag

SEQ ID NO: 671 - DNA, 47 bp, Homo sapiens
  source 1..47: mol_type = genomic DNA
cgatgagaccacggagccacccccagtgggtgccgggaccggaggag

SEQ ID NO: 672 - DNA, 48 bp, Homo sapiens
  source 1..48: mol_type = genomic DNA
ctgggaggtggcttacatggtggtgagcagaggggggtgtagtcgggg

SEQ ID NO: 673 - DNA, 50 bp, Homo sapiens
  source 1..50: mol_type = genomic DNA
ctaagagaaaagaaatatttggaggatattgaaagtgtgaaaaaaagaat

SEQ ID NO: 674 - DNA, 46 bp, Homo sapiens
  source 1..46: mol_type = genomic DNA
ccgagtgcaccaccatcccccctggaaacactgcaggaaacagggg

SEQ ID NO: 675 - DNA, 50 bp, Homo sapiens
  source 1..50: mol_type = genomic DNA
ctgggaggtggcttacatggtggtgagcagaggggggtgtagtcggggat

SEQ ID NO: 676 - DNA, 47 bp, Homo sapiens
  source 1..47: mol_type = genomic DNA
ctgaggaaaagctggtgacaggaagaggggtggcgtgacctgtggat

SEQ ID NO: 677 - DNA, 46 bp, Homo sapiens
  source 1..46: mol_type = genomic DNA
cgaggaagcgtcggtgacaggaagaggggtggtgtcacctgtggat

SEQ ID NO: 678 - DNA, 47 bp, Homo sapiens
  source 1..47: mol_type = genomic DNA
ccgaggaagcgtcggtgacaggaagaggggtggtgtcacctgtggat

SEQ ID NO: 679 - DNA, 47 bp, Homo sapiens
  source 1..47: mol_type = genomic DNA
ctgaggaagtgtcggtgacaggaagagtcgtggtgtcaccggtggat

SEQ ID NO: 680 - DNA, 49 bp, Homo sapiens
  source 1..49: mol_type = genomic DNA
cagggggaatgacccccactgtcttctccttccccacacactgcagggg

SEQ ID NO: 681 - DNA, 50 bp, Homo sapiens
  source 1..50: mol_type = genomic DNA
cagggggaatgacccccactgtcttctccttccccacacactgcaggggg

SEQ ID NO: 682 - DNA, 48 bp, Homo sapiens
  source 1..48: mol_type = genomic DNA
cgtggtgtcaccggtggatgctgaggaagcgccggtgacaggaagagt

SEQ ID NO: 683 - DNA, 51 bp, Homo sapiens
  source 1..51: mol_type = unassigned DNA
ccacgctggccacgctggccatcacgtaggcttcctggagggagggagagg

SEQ ID NO: 684-699 - skipped (16 entries)

SEQ ID NO: 700 - protein, 93 aa, synthetic construct
  REGION 1..93: note = polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIA

SEQ ID NO: 701 - protein, 77 aa, Escherichia coli
DATFGDTCSTHPLKEEIIKKRSETVKAKMLKLGPDGRKALYSKPGSKNGRWNPETHKFCKCGVRIQTSAYTCSKCRN

SEQ ID NO: 702 - protein, 170 aa, synthetic construct
  REGION 1..170: note = polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRN

SEQ ID NO: 703 - protein, 83 aa, synthetic construct
  REGION 1..83: note = polypeptide
DATFGDTCSTHPLKEEIIKKRSETVKAKMLKLGPDGRKALYSKPGSKNGRWNPETHKFCKCGVRIQTSAYTCSKCRNGGSGGS

SEQ ID NO: 704 - protein, 176 aa, synthetic construct
  REGION 1..176: note = polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNGGSGGS

SEQ ID NO: 705-709 - skipped (5 entries)

SEQ ID NO: 710 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 711 - protein, 1368 aa, Streptococcus pyogenes [sequence omitted]

SEQ ID NO: 712 - protein, 1082 aa, Neisseria meningitidis
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADKIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 713 - protein, 984 aa, Campylobacter jejuni
MARILAFDIGISSIGWAFSENDELKDCGVRIFTKAENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDGLKLIFKKQREFGFSFSKKFEEEVLSVAFYKRALKDFSHLVGNCSFFTDEKRAPKNSPLAFMFVALTRIINLLNNLKNTEGILYTKDDLNTLLNEVLKNGTLTYKQTKKLLGLSDDYEFKREKGTYFIEFKKYKEFIKALGEHNLSQDDLNEIAKDITLIKDEIKLKKALAKYDLNQNQIDSLSKLEFKDHLNISFKALKLITPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNKTPFEAFGNDSAKWQKIEVLAKNLPTKKQKRILDKNYKDKEQKDFKDRNLNDTRYIARLVLNYTKDYLDFLPLSDDENTKLNDTQKGSKVHVEAKSGMLTSALRHTWGFSTKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 714 - protein, 1377 aa, Streptococcus gallolyticus
  source 1..1377: note = subspecies: pasteurianus
MTKKNYSIGLDIGTNSVGWAVITDDYKVPAKKMKVLGNTDKKYIKKNLLGALLFDSGETAEATRLKRTARRRYTRRKNRLRYLQEIFAEEMTKVDESFFYRLDESFLTTDEKDFERHPIFGNKADEIKYHQEFPTIYHLRKHLADSSEKADLRLVYLALAHMIKFRGHFLIEGELNAENTDVQKIFADFVGVYDRTFDDSHLSEITVDAASILTEKISKSRRLENLIKYYPTEKKNTLFGNLIALALGLQPNFKMNFKLSEDAKLQFSKDSYNEDLEELLGKIGDDYADLFTSAKNLYDAILLSGILTVDDNSTKAPLSASMIKRYAEHHEDLEKLKEFIKANKSELYHDIFKDETKNGYAGYIENGVKQDEFYKYLKNTLSKIAGSDYFLDKIEREDFLRKQRTFDNGSIPHQIHLQEMHAILRRQGDYYPFLKENQDRIEKILTFRIPYYVGPLARKDSRFSWAEYHSDEKITPWNFDKVIDKEKSAEKFITRMTLNDLYLPEEKVLPKHSHVYETYAVYNELTKIKYVNEQGKDSFFDSNMKQEIFDHVFKENRKVTKEKLLNYLNKEFPEYRIKDLIGLDKENKSFNASLGTYHDLKKILDKAFLDDKVNEEVIEDIIKTLTLFEDKDMIHERLQKYSDIFTADQLKKLERRHYTGWGRLSYKLINGIRNKENNKTILDYLIDDGSANRNFMQLINDDTLPFKQIIQKSQVVGDVDDIEAVVHDLPGSPAIKKGILQSVKIVDELVKVMGDNPDNIVIEMARENQTTNRGRSQSQQRLKKLQNSLKELGSNILNEEKPSYIEDKVENSHLQNDQLFLYYIQNGKDMYTGDELDIDHLSDYDIDHIIPQAFIKDDSIDNRVLTSSAKNRGKSDDVPSLDIVRARKAEWVRLYKSGLISKRKFDNLTKAERGGLTEADKAGFIKRQLVETRQITKHVAQILDARFNTESDENDKVIRDVKVITLKSNLVSQFRKDFEFYKVREINDYHHAHDAYLNAVVGTALLKKYPKLASEFVYGEYKKYDVHKLIAKSSDDHSEMGKATAKYFFYSNLMNFFKRVIRYSNGKVIVRPVVEYSKDTEDIAWDKKSNFRTICKVLSYPQVNIVKKVETQTGGFSKESILPKGDSDKLIPRKTKKAYWDTKKYGGFDSPTVAYSVFVVADVEKGKAKKLKTVKELVGISIMERSFFEENPVEFLENKGYHNIREDKLIKLPKYSLFEFEGGKRRLLASASELQKGNEMVIPGHLVKLLYHAQRINSFNSTKYLDYVSAHKKEFEKVLSCVEDFANLYVDVEKNLSKIRAVADSMDNFSIEEISNSFINLLTLTALGAPADFNFLGEKIPRKRYTSTKECLNATLIHQSITGLYETRIDLSKIGEE

SEQ ID NO: 715 - protein, 1021 aa, Clostridium cellulolyticum
MKYTLGLDVGIASVGWAVIDKDNNKIIDLGVRCFDKAEESKTGESLATARRIARGMRRRISRRSQRLRLVKKLFVQYEIIKDSSEFNRIFDTSRDGWKDPWELRYNALSRILKPYELVQVLTHITKRRGFKSNRKEDLSTTKEGVVITSIKNNSEMLRTKNYRTIGEMIFMETPENSNKRNKVDEYIHTIAREDLLNEIKYIFSIQRKLGSPFVTEKLEHDFLNIWEFQRPFASGDSILSKVGKCTLLKEELRAPTSCYTSEYFGLLQSINNLVLVEDNNTLTLNNDQRAKIIEYAHFKNEIKYSEIRKLLDIEPEILFKAHNLTHKNPSGNNESKKFYEMKSYHKLKSTLPTDIWGKLHSNKESLDNLFYCLTVYKNDNEIKDYLQANNLDYLIEYIAKLPTFNKFKHLSLVAMKRIIPFMEKGYKYSDACNMAELDFTGSSKLEKCNKLTVEPIIENVTNPVVIRALTQARKVINAIIQKYGLPYMVNIELAREAGMTRQDRDNLKKEHENNRKAREKISDLIRQNGRVASGLDILKWRLWEDQGGRCAYSGKPIPVCDLLNDSLTQIDHIYPYSRSMDDSYMNKVLVLTDENQNKRSYTPYEVWGSTEKWEDFEARIYSMHLPQSKEKRLLNRNFITKDLDSFISRNLNDTRYISRFLKNYIESYLQFSNDSPKSCVVCVNGQCTAQLRSRWGLNKNREESDLHHALDAAVIACADRKIIKEITNYYNERENHNYKVKYPLPWHSFRQDLMETLAGVFISRAPRRKITGPAHDETIRSPKHFNKGLTSVKIPLTTVTLEKLETMVKNTKGGISDKAVYNVLKNRLIEHNNKPLKAFAEKIYKPLKNGTNGAIIRSIRVETPSYTGVFRNEGKGISDNSLMVRVDVFKKKDKYYLVPIYVAHMIKKELPSKAIVPLKPESQWELIDSTHEFLFSLYQNDYLVIKTKKGITEGYYRSCHRGTGSLSLMPHFANNKNVKIDIGVRTAISIEKYNVDILGNKSIVKGEPRRGMEKYNSFKSN

SEQ ID NO: 716 - protein, 1082 aa, Geobacillus thermodenitrificans
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 717-719 - skipped (3 entries)

SEQ ID NO: 720 - protein, 1306 aa, Acidaminococcus sp.
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 721 - protein, 977 aa, unidentified
  REGION 1..977: note = Planctomycetes bacterium sequence
QEIKRINKIRRRLVKDSNTKKAGKTGPMKTLLVRVMTPDLRERLENLRKKPENIPQPISNTSRANLNKLLTDYTEMKKAILHVYWEEFQKDPVGLMSRVAQPAPKNIDQRKLIPVKDGNERLTSSGFACSQCCQPLYVYKLEQVNDKGKPHTNYFGRCNVSEHERLILLSPHKPEANDELVTYSLGKFGQRALDFYSIHVTRESNHPVKPLEQIGGNSCASGPVGKALSDACMGAVASFLTKYQDIILEHQKVIKKNEKRLANLKDIASANGLAFPKITLPPQPHTKEGIEAYNNVVAQIVIWVNLNLWQKLKIGRDEAKPLQRLKGFPSFPLVERQANEVDWWDMVCNVKKLINEKKEDGKVFWQNLAGYKRQEALLPYLSSEEDRKKGKKFARYQFGDLLLHLEKKHGEDWGKVYDEAWERIDKKVEGLSKHIKLEEERRSEDAQSKAALTDWLRAKASFVIEGLKEADKDEFCRCELKLQKWYGDLRGKPFAIEAENSILDISGFSKQYNCAFIWQKDGVKKLNLYLIINYFKGGKLRFKKIKPEAFEANRFYTVINKKSGEIVPMEVNFNFDDPNLIILPLAFGKRQGREFIWNDLLSLETGSLKLANGRVIEKTLYNRRTRQDEPALFVALTFERREVLDSSNIKPMNLIGIDRGENIPAVIALTDPEGCPLSRFKDSLGNPTHILRIGESYKEKQRTIQAAKEVEQRRAGGYSRKYASKAKNLADDMVRNTARDLLYYAVTQDAMLIFENLSRGFGRQGKRTFMAERQYTRMEDWLTAKLAYEGLPSKTYLSKTLAQYTSKTCSNCGFTITSADYDRVLEKLKKTATGWMTTINGKELKVEGQITYYNRYKRQNVVKDLSVELDRLSEESVNNDISSWTKGRSGEALSLLKKRFSHRPVQEKFVCLNCGFETHADEQAALNIARSWLFLRSQEYKKYQTNKTTGNTDKRAFVETWQSFYRKKLKEVWKPAV

SEQ ID NO: 722-729 - skipped (8 entries)

SEQ ID NO: 730 - protein, 1223 aa, synthetic construct
  REGION 1..1223: note = polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNMKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 731 - protein, 1538 aa, synthetic construct
  REGION 1..1538: note = polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 732 - protein, 1252 aa, synthetic construct
  REGION 1..1252: note = polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNMAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADKIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 733 - protein, 1154 aa, synthetic construct
  REGION 1..1154: note = polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNMARILAFDIGISSIGWAFSENDELKDCGVRIFTKAENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDGLKLIFKKQREFGFSFSKKFEEEVLSVAFYKRALKDFSHLVGNCSFFTDEKRAPKNSPLAFMFVALTRIINLLNNLKNTEGILYTKDDLNTLLNEVLKNGTLTYKQTKKLLGLSDDYEFKREKGTYFIEFKKYKEFIKALGEHNLSQDDLNEIAKDITLIKDEIKLKKALAKYDLNQNQIDSLSKLEFKDHLNISFKALKLITPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNKTPFEAFGNDSAKWQKIEVLAKNLPTKKQKRILDKNYKDKEQKDFKDRNLNDTRYIARLVLNYTKDYLDFLPLSDDENTKLNDTQKGSKVHVEAKSGMLTSALRHTWGFSTKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 734 - protein, 1547 aa, synthetic construct
  REGION 1..1547: note = polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNMTKKNYSIGLDIGTNSVGWAVITDDYKVPAKKMKVLGNTDKKYIKKNLLGALLFDSGETAEATRLKRTARRRYTRRKNRLRYLQEIFAEEMTKVDESFFYRLDESFLTTDEKDFERHPIFGNKADEIKYHQEFPTIYHLRKHLADSSEKADLRLVYLALAHMIKFRGHFLIEGELNAENTDVQKIFADFVGVYDRTFDDSHLSEITVDAASILTEKISKSRRLENLIKYYPTEKKNTLFGNLIALALGLQPNFKMNFKLSEDAKLQFSKDSYNEDLEELLGKIGDDYADLFTSAKNLYDAILLSGILTVDDNSTKAPLSASMIKRYAEHHEDLEKLKEFIKANKSELYHDIFKDETKNGYAGYIENGVKQDEFYKYLKNTLSKIAGSDYFLDKIEREDFLRKQRTFDNGSIPHQIHLQEMHAILRRQGDYYPFLKENQDRIEKILTFRIPYYVGPLARKDSRFSWAEYHSDEKITPWNFDKVIDKEKSAEKFITRMTLNDLYLPEEKVLPKHSHVYETYAVYNELTKIKYVNEQGKDSFFDSNMKQEIFDHVFKENRKVTKEKLLNYLNKEFPEYRIKDLIGLDKENKSFNASLGTYHDLKKILDKAFLDDKVNEEVIEDIIKTLTLFEDKDMIHERLQKYSDIFTADQLKKLERRHYTGWGRLSYKLINGIRNKENNKTILDYLIDDGSANRNFMQLINDDTLPFKQIIQKSQVVGDVDDIEAVVHDLPGSPAIKKGILQSVKIVDELVKVMGDNPDNIVIEMARENQTTNRGRSQSQQRLKKLQNSLKELGSNILNEEKPSYIEDKVENSHLQNDQLFLYYIQNGKDMYTGDELDIDHLSDYDIDHIIPQAFIKDDSIDNRVLTSSAKNRGKSDDVPSLDIVRARKAEWVRLYKSGLISKRKFDNLTKAERGGLTEADKAGFIKRQLVETRQITKHVAQILDARFNTESDENDKVIRDVKVITLKSNLVSQFRKDFEFYKVREINDYHHAHDAYLNAVVGTALLKKYPKLASEFVYGEYKKYDVHKLIAKSSDDHSEMGKATAKYFFYSNLMNFFKRVIRYSNGKVIVRPVVEYSKDTEDIAWDKKSNFRTICKVLSYPQVNIVKKVETQTGGFSKESILPKGDSDKLIPRKTKKAYWDTKKYGGFDSPTVAYSVFVVADVEKGKAKKLKTVKELVGISIMERSFFEENPVEFLENKGYHNIREDKLIKLPKYSLFEFEGGKRRLLASASELQKGNEMVIPGHLVKLLYHAQRINSFNSTKYLDYVSAHKKEFEKVLSCVEDFANLYVDVEKNLSKIRAVADSMDNFSIEEISNSFINLLTLTALGAPADFNFLGEKIPRKRYTSTKECLNATLIHQSITGLYETRIDLSKIGEE

SEQ ID NO: 735 - protein, 1191 aa, synthetic construct
  REGION 1..1191: note = polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNMKYTLGLDVGIASVGWAVIDKDNNKIIDLGVRCFDKAEESKTGESLATARRIARGMRRRISRRSQRLRLVKKLFVQYEIIKDSSEFNRIFDTSRDGWKDPWELRYNALSRILKPYELVQVLTHITKRRGFKSNRKEDLSTTKEGVVITSIKNNSEMLRTKNYRTIGEMIFMETPENSNKRNKVDEYIHTIAREDLLNEIKYIFSIQRKLGSPFVTEKLEHDFLNIWEFQRPFASGDSILSKVGKCTLLKEELRAPTSCYTSEYFGLLQSINNLVLVEDNNTLTLNNDQRAKIIEYAHFKNEIKYSEIRKLLDIEPEILFKAHNLTHKNPSGNNESKKFYEMKSYHKLKSTLPTDIWGKLHSNKESLDNLFYCLTVYKNDNEIKDYLQANNLDYLIEYIAKLPTFNKFKHLSLVAMKRIIPFMEKGYKYSDACNMAELDFTGSSKLEKCNKLTVEPIIENVTNPVVIRALTQARKVINAIIQKYGLPYMVNIELAREAGMTRQDRDNLKKEHENNRKAREKISDLIRQNGRVASGLDILKWRLWEDQGGRCAYSGKPIPVCDLLNDSLTQIDHIYPYSRSMDDSYMNKVLVLTDENQNKRSYTPYEVWGSTEKWEDFEARIYSMHLPQSKEKRLLNRNFITKDLDSFISRNLNDTRYISRFLKNYIESYLQFSNDSPKSCVVCVNGQCTAQLRSRWGLNKNREESDLHHALDAAVIACADRKIIKEITNYYNERENHNYKVKYPLPWHSFRQDLMETLAGVFISRAPRRKITGPAHDETIRSPKHFNKGLTSVKIPLTTVTLEKLETMVKNTKGGISDKAVYNVLKNRLIEHNNKPLKAFAEKIYKPLKNGTNGAIIRSIRVETPSYTGVFRNEGKGISDNSLMVRVDVFKKKDKYYLVPIYVAHMIKKELPSKAIVPLKPESQWELIDSTHEFLFSLYQNDYLVIKTKKGITEGYYRSCHRGTGSLSLMPHFANNKNVKIDIGVRTAISIEKYNVDILGNKSIVKGEPRRGMEKYNSFKSN

SEQ ID NO: 736 - protein, 1252 aa, synthetic construct
  REGION 1..1252: note = polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNMKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 737-739 - skipped (3 entries)

SEQ ID NO: 740 - protein, 7 aa, Simian virus 40
PKKKRKV

SEQ ID NO: 741 - protein, 16 aa, unidentified
  REGION 1..16: note = Nucleoplasmin bipartite NLS sequence
KRPAATKKAGQAKKKK

SEQ ID NO: 742 - protein, 30 aa, unidentified
  REGION 1..30: note = Nuclear localization signal sequence
MDSLLMNRRKFLYQFKNVRWAKGRRETYLC

SEQ ID NO: 743 - protein, 18 aa, unidentified
  REGION 1..18: note = Nuclear localization signal sequence
KRTADGSEFESPKKKRKV

SEQ ID NO: 744 - protein, 17 aa, unidentified
  REGION 1..17: note = Nuclear localization signal sequence
KRTADGSEFEPKKKRKV

SEQ ID NO: 745 - protein, 19 aa, unidentified
  REGION 1..19: note = Nuclear localization signal sequence
NLSKRPAAIKKAGQAKKKK

SEQ ID NO: 746 - protein, 11 aa, unidentified
  REGION 1..11: note = Nuclear localization signal sequence
RQRRNELKRSF

SEQ ID NO: 747 - protein, 38 aa, Homo sapiens
NQSSNFGPMKGGNFGGRSSGPYGGGGQYFAKPRNQGGY

SEQ ID NO: 748 - protein, 16 aa, synthetic construct
  REGION 1..16: note = peptide
  MOD_RES 3..12: note = Any amino acid
KRXXXXXXXXXXKKKL

SEQ ID NO: 749 - skipped

SEQ ID NO: 750 - protein, 1229 aa, synthetic construct
  REGION 1..1229: note = polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNGGSGGSMKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 751 - protein, 1544 aa, synthetic construct
  REGION 1..1544: note = polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNGGSGGSMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 752 - protein, 1258 aa, synthetic construct
  REGION 1..1258: note = polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNGGSGGSMAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADKIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 753 - protein, 1160 aa, synthetic construct
  REGION 1..1160: note = polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNGGSGGSMARILAFDIGISSIGWAFSENDELKDCGVRIFTKAENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDGLKLIFKKQREFGFSFSKKFEEEVLSVAFYKRALKDFSHLVGNCSFFTDEKRAPKNSPLAFMFVALTRIINLLNNLKNTEGILYTKDDLNTLLNEVLKNGTLTYKQTKKLLGLSDDYEFKREKGTYFIEFKKYKEFIKALGEHNLSQDDLNEIAKDITLIKDEIKLKKALAKYDLNQNQIDSLSKLEFKDHLNISFKALKLITPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNKTPFEAFGNDSAKWQKIEVLAKNLPTKKQKRILDKNYKDKEQKDFKDRNLNDTRYIARLVLNYTKDYLDFLPLSDDENTKLNDTQKGSKVHVEAKSGMLTSALRHTWGFSTKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 754 - protein, 1553 aa, synthetic construct
  REGION 1..1553: note = polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNGGSGGSMTKKNYSIGLDIGTNSVGWAVITDDYKVPAKKMKVLGNTDKKYIKKNLLGALLFDSGETAEATRLKRTARRRYTRRKNRLRYLQEIFAEEMTKVDESFFYRLDESFLTTDEKDFERHPIFGNKADEIKYHQEFPTIYHLRKHLADSSEKADLRLVYLALAHMIKFRGHFLIEGELNAENTDVQKIFADFVGVYDRTFDDSHLSEITVDAASILTEKISKSRRLENLIKYYPTEKKNTLFGNLIALALGLQPNFKMNFKLSEDAKLQFSKDSYNEDLEELLGKIGDDYADLFTSAKNLYDAILLSGILTVDDNSTKAPLSASMIKRYAEHHEDLEKLKEFIKANKSELYHDIFKDETKNGYAGYIENGVKQDEFYKYLKNTLSKIAGSDYFLDKIEREDFLRKQRTFDNGSIPHQIHLQEMHAILRRQGDYYPFLKENQDRIEKILTFRIPYYVGPLARKDSRFSWAEYHSDEKITPWNFDKVIDKEKSAEKFITRMTLNDLYLPEEKVLPKHSHVYETYAVYNELTKIKYVNEQGKDSFFDSNMKQEIFDHVFKENRKVTKEKLLNYLNKEFPEYRIKDLIGLDKENKSFNASLGTYHDLKKILDKAFLDDKVNEEVIEDIIKTLTLFEDKDMIHERLQKYSDIFTADQLKKLERRHYTGWGRLSYKLINGIRNKENNKTILDYLIDDGSANRNFMQLINDDTLPFKQIIQKSQVVGDVDDIEAVVHDLPGSPAIKKGILQSVKIVDELVKVMGDNPDNIVIEMARENQTTNRGRSQSQQRLKKLQNSLKELGSNILNEEKPSYIEDKVENSHLQNDQLFLYYIQNGKDMYTGDELDIDHLSDYDIDHIIPQAFIKDDSIDNRVLTSSAKNRGKSDDVPSLDIVRARKAEWVRLYKSGLISKRKFDNLTKAERGGLTEADKAGFIKRQLVETRQITKHVAQILDARFNTESDENDKVIRDVKVITLKSNLVSQFRKDFEFYKVREINDYHHAHDAYLNAVVGTALLKKYPKLASEFVYGEYKKYDVHKLIAKSSDDHSEMGKATAKYFFYSNLMNFFKRVIRYSNGKVIVRPVVEYSKDTEDIAWDKKSNFRTICKVLSYPQVNIVKKVETQTGGFSKESILPKGDSDKLIPRKTKKAYWDTKKYGGFDSPTVAYSVFVVADVEKGKAKKLKTVKELVGISIMERSFFEENPVEFLENKGYHNIREDKLIKLPKYSLFEFEGGKRRLLASASELQKGNEMVIPGHLVKLLYHAQRINSFNSTKYLDYVSAHKKEFEKVLSCVEDFANLYVDVEKNLSKIRAVADSMDNFSIEEISNSFINLLTLTALGAPADFNFLGEKIPRKRYTSTKECLNATLIHQSITGLYETRIDLSKIGEE

SEQ ID NO: 755 - protein, 1197 aa, synthetic construct
  REGION 1..1197: note = polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNGGSGGSMKYTLGLDVGIASVGWAVIDKDNNKIIDLGVRCFDKAEESKTGESLATARRIARGMRRRISRRSQRLRLVKKLFVQYEIIKDSSEFNRIFDTSRDGWKDPWELRYNALSRILKPYELVQVLTHITKRRGFKSNRKEDLSTTKEGVVITSIKNNSEMLRTKNYRTIGEMIFMETPENSNKRNKVDEYIHTIAREDLLNEIKYIFSIQRKLGSPFVTEKLEHDFLNIWEFQRPFASGDSILSKVGKCTLLKEELRAPTSCYTSEYFGLLQSINNLVLVEDNNTLTLNNDQRAKIIEYAHFKNEIKYSEIRKLLDIEPEILFKAHNLTHKNPSGNNESKKFYEMKSYHKLKSTLPTDIWGKLHSNKESLDNLFYCLTVYKNDNEIKDYLQANNLDYLIEYIAKLPTFNKFKHLSLVAMKRIIPFMEKGYKYSDACNMAELDFTGSSKLEKCNKLTVEPIIENVTNPVVIRALTQARKVINAIIQKYGLPYMVNIELAREAGMTRQDRDNLKKEHENNRKAREKISDLIRQNGRVASGLDILKWRLWEDQGGRCAYSGKPIPVCDLLNDSLTQIDHIYPYSRSMDDSYMNKVLVLTDENQNKRSYTPYEVWGSTEKWEDFEARIYSMHLPQSKEKRLLNRNFITKDLDSFISRNLNDTRYISRFLKNYIESYLQFSNDSPKSCVVCVNGQCTAQLRSRWGLNKNREESDLHHALDAAVIACADRKIIKEITNYYNERENHNYKVKYPLPWHSFRQDLMETLAGVFISRAPRRKITGPAHDETIRSPKHFNKGLTSVKIPLTTVTLEKLETMVKNTKGGISDKAVYNVLKNRLIEHNNKPLKAFAEKIYKPLKNGTNGAIIRSIRVETPSYTGVFRNEGKGISDNSLMVRVDVFKKKDKYYLVPIYVAHMIKKELPSKAIVPLKPESQWELIDSTHEFLFSLYQNDYLVIKTKKGITEGYYRSCHRGTGSLSLMPHFANNKNVKIDIGVRTAISIEKYNVDILGNKSIVKGEPRRGMEKYNSFKSN

SEQ ID NO: 756 - protein, 1258 aa, synthetic construct
  REGION 1..1258: note = polypeptide
MGKSGIYQIKNTLNNKVYVGSAKDFEKRWKRHFKDLEKGCHSSIKLQRSFNKHGNVFECSILEEIPYEKDLIIERENFWIKELNSKINGYNIADATFGDTCSTHPLKEEIIKKRSETVKAKMLKLGPDGRKALYSKPGSKNGRWNPETHKFCKCGVRIQTSAYTCSKCRNGGSGGSMKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 757-779 - skipped (23 entries)

SEQ ID NO: 780 - DNA, 144 bp, Homo sapiens
  source 1..144: mol_type = unassigned DNA
accggtggatgccgaggaagcgtcggtgacaggaagaggggtggtgtcacctgtggatactgaggaaaagctggtgacaggaagaggggtggcgtgacctgtggatactgaggaagtgtcggtgacaggaagagtcgtggtgtc

SEQ ID NO: 781-879 - skipped (99 entries)

SEQ ID NO: 880 - protein, 7 aa, Simian virus 40
PKKKRKV

SEQ ID NO: 881 - protein, 19 aa, unidentified
  REGION 1..19: note = Nuclear localization signal sequence
MKRTADGSEFESPKKKRKV

SEQ ID NO: 882 - protein, 30 aa, unidentified; identical to SEQ ID NO: 742
  REGION 1..30: note = Nuclear localization signal sequence

SEQ ID NO: 883 - protein, 20 aa, unidentified
  REGION 1..20: note = Nucleoplasmin NLS sequence
AVKRPAATKKAGQAKKKKLD

SEQ ID NO: 884 - protein, 25 aa, unidentified
  REGION 1..25: note = EGL-13 NLS sequence
MSRRRKANPTKLSENAKKLAKEVEN

SEQ ID NO: 885 - protein, 9 aa, unidentified
  REGION 1..9: note = C-myc NLS sequence
PAAKRVKLD

SEQ ID NO: 886 - protein, 9 aa, unidentified
  REGION 1..9: note = Tus-protein NLS sequence
KLKIKRPVK

SEQ ID NO: 887 - protein, 8 aa, unidentified
  REGION 1..8: note = Myoma T protein sequence
VSRKRPRP

SEQ ID NO: 888 - protein, 10 aa, unidentified
  REGION 1..10: note = Hepatitis D virus antigen NLS sequence
EGAPPAKRAR

SEQ ID NO: 889 - protein, 12 aa, unidentified
  REGION 1..12: note = Murine p53 NLS sequence
PPQPKKKPLDGE

SEQ ID NO: 890 - protein, 21 aa, unidentified
  REGION 1..21: note = PE1 and PE2 NLS sequence
SGGSKRTADGSEFEPKKKRKV

SEQ ID NO: 891-900 - skipped (10 entries)

SEQ ID NO: 901 - protein, 5 aa, synthetic construct
  REGION 1..5: note = peptide
GSGGS

SEQ ID NO: 902 - protein, 5 aa, synthetic construct
  REGION 1..5: note = peptide
GGGGS

SEQ ID NO: 903 - protein, 4 aa, synthetic construct
  REGION 1..4: note = peptide
GGGS

SEQ ID NO: 904 - protein, 7 aa, synthetic construct
  REGION 1..7: note = peptide
AAEPKSS

SEQ ID NO: 905 - protein, 4 aa, synthetic construct
  REGION 1..4: note = peptide
GGGG

SEQ ID NO: 906 - protein, 14 aa, synthetic construct
  REGION 1..14: note = peptide
GGGGDKTHTCPPCP

SEQ ID NO: 907-1000 - skipped (94 entries)

SEQ ID NO: 1001 - DNA, 21 bp, synthetic construct
  misc_feature 1..21: note = oligonucleotide
  source 1..21: mol_type = other DNA
ccaaacaaaagtgatctcctc

SEQ ID NO: 1002 - DNA, 21 bp, synthetic construct
  misc_feature 1..21: note = oligonucleotide
  source 1..21: mol_type = other DNA
aaattcttccctttctgcttc

SEQ ID NO: 1003 - DNA, 21 bp, synthetic construct
  misc_feature 1..21: note = oligonucleotide
  source 1..21: mol_type = other DNA
taagagcaatcagtgaggaat

SEQ ID NO: 1004 - DNA, 21 bp, synthetic construct
  misc_feature 1..21: note = oligonucleotide
  source 1..21: mol_type = other DNA
tggcccagggacagctcagtg

SEQ ID NO: 1005 - DNA, 21 bp, synthetic construct
  misc_feature 1..21: note = oligonucleotide
  source 1..21: mol_type = other DNA
cctctctctaaaatcactgag

SEQ ID NO: 1006 - DNA, 21 bp, synthetic construct
  misc_feature 1..21: note = oligonucleotide
  source 1..21: mol_type = other DNA
cctctctctgaaatcactgag

SEQ ID NO: 1007 - DNA, 21 bp, synthetic construct
  misc_feature 1..21: note = oligonucleotide
  source 1..21: mol_type = other DNA
cctctctctgaaatcactaag

SEQ ID NO: 1008 - DNA, 21 bp, synthetic construct
  misc_feature 1..21: note = oligonucleotide
  source 1..21: mol_type = other DNA
cctctctctgaaatcactgcg

SEQ ID NO: 1009 - DNA, 21 bp, synthetic construct
  misc_feature 1..21: note = oligonucleotide
  source 1..21: mol_type = other DNA
aaaataggcgtctcaggcaga

SEQ ID NO: 1010 - DNA, 21 bp, synthetic construct
  misc_feature 1..21: note = oligonucleotide
  source 1..21: mol_type = other DNA
tggcgtgacctgtggatactg

SEQ ID NO: 1011 - DNA, 21 bp, synthetic construct
  misc_feature 1..21: note = oligonucleotide
  source 1..21: mol_type = other DNA
tggcgtgacctgtgggcactg

SEQ ID NO: 1012 - DNA, 21 bp, synthetic construct
  misc_feature 1..21: note = oligonucleotide
  source 1..21: mol_type = other DNA
gaggctgcggcccaccgcacc

SEQ ID NO: 1013 - DNA, 21 bp, synthetic construct
  misc_feature 1..21: note = oligonucleotide
  source 1..21: mol_type = other DNA
aaaaataggcgtctcaggcag

SEQ ID NO: 1014 - DNA, 21 bp, synthetic construct
  misc_feature 1..21: note = oligonucleotide
  source 1..21: mol_type = other DNA
ggccagacctaagagcaatca

SEQ ID NO: 1015 - DNA, 21 bp, synthetic construct
  misc_feature 1..21: note = oligonucleotide
  source 1..21: mol_type = other DNA
agcaagcaggacaagaagcgg

SEQ ID NO: 1016 - DNA, 21 bp, synthetic construct
  misc_feature 1..21: note = oligonucleotide
  source 1..21: mol_type = other DNA
aggctgcggcccaccgcacca

SEQ ID NO: 1017 - DNA, 21 bp, synthetic construct
  misc_feature 1..21: note = oligonucleotide
  source 1..21: mol_type = other DNA
gaggagaccggaaaatgggag

SEQ ID NO: 1018 - DNA, 21 bp, synthetic construct
  misc_feature 1..21: note = oligonucleotide
  source 1..21: mol_type = other DNA
aggttgggcaatggtagagta

SEQ ID NO: 1019 - DNA, 21 bp, synthetic construct
  misc_feature 1..21: note = oligonucleotide
  source 1..21: mol_type = other DNA
agaaaggaggagtggggatgg

SEQ ID NO: 1020 - DNA, 21 bp, synthetic construct
  misc_feature 1..21: note = oligonucleotide
  source 1..21: mol_type = other DNA
agctgctgtgggccaccgctg

SEQ ID NO: 1021 - DNA, 21 bp, synthetic construct
  misc_feature 1..21: note = oligonucleotide
  source 1..21: mol_type = other DNA
acttacagtgtctgaagaagt

SEQ ID NO: 1022 - DNA, 21 bp, synthetic construct
  misc_feature 1..21: note = oligonucleotide
  source 1..21: mol_type = other DNA
ggaagaaaggaggagtgggga

SEQ ID NO: 1023 - DNA, 21 bp, synthetic construct
  misc_feature 1..21: note = oligonucleotide
  source 1..21: mol_type = other DNA
ccatcctcaccatcatcacac

SEQ ID NO: 1024 - DNA, 21 bp, synthetic construct
  misc_feature 1..21: note = oligonucleotide
  source 1..21: mol_type = other DNA
tagtcaaatccgtcagtgatc

SEQ ID NO: 1025 - DNA, 21 bp, synthetic construct
  misc_feature 1..21: note = oligonucleotide
  source 1..21: mol_type = other DNA
gcccttttttgggttttcggc

SEQ ID NO: 1026 - DNA, 21 bp, synthetic construct
  misc_feature 1..21: note = oligonucleotide
  source 1..21: mol_type = other DNA
aaaataggcgtctcaggcaga

SEQ ID NO: 1027 - DNA, 21 bp, synthetic construct
  misc_feature 1..21: note = oligonucleotide
  source 1..21: mol_type = other DNA
ccatcctcaccatcatcacac

SEQ ID NO: 1028 - DNA, 21 bp, synthetic construct
  misc_feature 1..21: note = oligonucleotide
  source 1..21: mol_type = other DNA
aagcaagcaggacaagaagcg

SEQ ID NO: 1029 - DNA, 21 bp, synthetic construct
  misc_feature 1..21: note = oligonucleotide
  source 1..21: mol_type = other DNA
aatctttaataaaattacata

SEQ ID NO: 1030 - DNA, 21 bp, synthetic construct
  misc_feature 1..21: note = oligonucleotide
  source 1..21: mol_type = other DNA
gccactgccacagctccttct

SEQ ID NO: 1031 - DNA, 21 bp, synthetic construct
  misc_feature 1..21: note = oligonucleotide
  source 1..21: mol_type = other DNA
gccactaccacagctccttct

SEQ ID NO: 1032 - DNA, 21 bp, synthetic construct
  misc_feature 1..21: note = oligonucleotide
  source 1..21: mol_type = other DNA
aaaaataggcgtctcaggcag

SEQ ID NO: 1033 - DNA, 21 bp, synthetic construct
  misc_feature 1..21: note = oligonucleotide
  source 1..21: mol_type = other DNA
gagtcacaggtaagtgctaaa

SEQ ID NO: 1034 - DNA, 21 bp, synthetic construct
  misc_feature 1..21: note = oligonucleotide
  source 1..21: mol_type = other DNA
gccactaccacagctcctcct

SEQ ID NO: 1035 - DNA, 21 bp, synthetic construct
  misc_feature 1..21: note = oligonucleotide
  source 1..21: mol_type = other DNA
caaaacatcttgttgagggca

SEQ ID NO: 1036 - DNA, 21 bp, synthetic construct
  misc_feature 1..21: note = oligonucleotide
  source 1..21: mol_type = other DNA
tggaatcaacccacagctgca

SEQ ID NO: 1037 - DNA, 21 bp, synthetic construct
  misc_feature 1..21: note = oligonucleotide
  source 1..21: mol_type = other DNA
ctttgttgatgtttcaataaa

SEQ ID NO: 1038 - DNA, 21 bp, synthetic construct
  misc_feature 1..21: note = oligonucleotide
  source 1..21: mol_type = other DNA
tagtcaaatccgtcagtgatc

SEQ ID NO: 1039 - DNA, 21 bp, synthetic construct
  misc_feature 1..21: note = oligonucleotide
  source 1..21: mol_type = other DNA
ggccagacctaagagcaatca

SEQ ID NO: 1040 - DNA, 21 bp, synthetic construct
  misc_feature 1..21: note = oligonucleotide
  source 1..21: mol_type = other DNA
ggcaaaacatcttgttgaggg

SEQ ID NO: 1041 - DNA, 21 bp, synthetic construct
  misc_feature 1..21: note = oligonucleotide
  source 1..21: mol_type = other DNA
tatgaagaacagaaatggagg

SEQ ID NO: 1042 - DNA, 21 bp, synthetic construct
  misc_feature 1..21: note = oligonucleotide
  source 1..21: mol_type = other DNA
ggtcgagaggagtacagtgca

SEQ ID NO: 1043 - DNA, 21 bp, synthetic construct
  misc_feature 1..21: note = oligonucleotide
  source 1..21: mol_type = other DNA
cttacctcacttagtgctccc

SEQ ID NO: 1044 - DNA, 21 bp, synthetic construct
  misc_feature 1..21: note = oligonucleotide
  source 1..21: mol_type = other DNA
ccatcctcaccatcatcacac

SEQ ID NO: 1045 - DNA, 21 bp, synthetic construct
  misc_feature 1..21: note = oligonucleotide
  source 1..21: mol_type = other DNA
gattttgggcgggccaaactg

SEQ ID NO: 1046 - DNA, 21 bp, synthetic construct
  misc_feature 1..21: note = oligonucleotide
  source 1..21: mol_type = other DNA
tccgtagaaaggtagttgtct

SEQ ID NO: 1047 - DNA, 21 bp, synthetic construct
  misc_feature 1..21: note = oligonucleotide
  source 1..21: mol_type = other DNA
gttagtcccttctgaaattca

SEQ ID NO: 1048 - DNA, 21 bp, synthetic construct
  misc_feature 1..21: note = oligonucleotide
  source 1..21: mol_type = other DNA
tgaagaggggtggcgtgacct

SEQ ID NO: 1049 - DNA, 21 bp, synthetic construct
  misc_feature 1..21: note = oligonucleotide
  source 1..21: mol_type = other DNA
ccatcctcaccatcatcacac

SEQ ID NO: 1050 - DNA, 21 bp, synthetic construct
  misc_feature 1..21: note = oligonucleotide
  source 1..21: mol_type = other DNA
ccatcctcaccatcatcacac

SEQ ID NO: 1051 - DNA, 21 bp, synthetic construct
  misc_feature 1..21: note = oligonucleotide
  source 1..21: mol_type = other DNA
ggtctagaggagtacagtgca

SEQ ID NO: 1052 - DNA, 21 bp, synthetic construct
  misc_feature 1..21: note = oligonucleotide
  source 1..21: mol_type = other DNA
ggtcacgaggagtacagtgca

SEQ ID NO: 1053 - DNA, 21 bp, synthetic construct
  misc_feature 1..21: note = oligonucleotide
  source 1..21: mol_type = other DNA
atgaagaacagaaatggaggt

SEQ ID NO: 1054 - DNA, 21 bp, synthetic construct
  misc_feature 1..21: note = oligonucleotide
  source 1..21: mol_type = other DNA
cccgtcatgtgggtcctgaat

SEQ ID NO: 1055 - DNA, 21 bp, synthetic construct
  misc_feature 1..21: note = oligonucleotide
  source 1..21: mol_type = other DNA
accagctcctctccctagcca

SEQ ID NO: 1056 - DNA, 21 bp, synthetic construct
  misc_feature 1..21: note = oligonucleotide
  source 1..21: mol_type = other DNA
aagtcacagacttggctgtcc

SEQ ID NO: 1057 - DNA, 21 bp, synthetic construct
  misc_feature 1..21: note = oligonucleotide
  source 1..21: mol_type = other DNA
gcttacctcacttagtgctcc

SEQ ID NO: 1058 - DNA, 21 bp, synthetic construct
  misc_feature 1..21: note = oligonucleotide
  source 1..21: mol_type = other DNA
aggaagagaatctccgcaaga

SEQ ID NO: 1059 - DNA, 21 bp, synthetic construct
  misc_feature 1..21: note = oligonucleotide
  source 1..21: mol_type = other DNA
ggcccctcctcagcgtcttat

SEQ ID NO: 1060 - DNA, 21 bp, synthetic construct
  misc_feature 1..21: note = oligonucleotide
  source 1..21: mol_type = other DNA
ggcccctcctcagcatcgtat

SEQ ID NO: 1061 - DNA, 21 bp, synthetic construct
  misc_feature 1..21: note = oligonucleotide
  source 1..21: mol_type = other DNA
aggaagagaatctccgcaaga

SEQ ID NO: 1062 - DNA, 21 bp, synthetic construct
  misc_feature 1..21: note = oligonucleotide
  source 1..21: mol_type = other DNA
agtgcaatgagggaccagtac

SEQ ID NO: 1063 - DNA, 21 bp, synthetic construct
  misc_feature 1..21: note = oligonucleotide
  source 1..21: mol_type = other DNA
agtgcaatgagggaccagtac

SEQ ID NO: 1064 - DNA, 21 bp, synthetic construct
  misc_feature 1..21: note = oligonucleotide
  source 1..21: mol_type = other DNA
tctgagcagcgctcatggtgg

SEQ ID NO: 1065 - DNA, 21 bp, synthetic construct
  misc_feature 1..21: note = oligonucleotide
  source 1..21: mol_type = other DNA
gaagagaatctccgcaagaaa

SEQ ID NO: 1066 - DNA, 21 bp, synthetic construct
  misc_feature 1..21: note = oligonucleotide
  source 1..21: mol_type = other DNA
aggaagagaatctccgcaaga

SEQ ID NO: 1067 - DNA, 21 bp, synthetic construct
  misc_feature 1..21: note = oligonucleotide
  source 1..21: mol_type = other DNA
ggaagaggggtggcctgacct

SEQ ID NO: 1068 - DNA, 21 bp, synthetic construct
  misc_feature 1..21: note = oligonucleotide
  source 1..21: mol_type = other DNA
gaagggactaactgagatgta

SEQ ID NO: 1069 - DNA, 21 bp, synthetic construct
  misc_feature 1..21: note = oligonucleotide
  source 1..21: mol_type = other DNA
ggcccctcctcagcatcttac

SEQ ID NO: 1070 - DNA, 21 bp, synthetic construct
  misc_feature 1..21: note = oligonucleotide
  source 1..21: mol_type = other DNA
ccggtggctgggccactctgc

SEQ ID NO: 1071 - DNA, 21 bp, synthetic construct
  misc_feature 1..21: note = oligonucleotide
  source 1..21: mol_type = other DNA
ggcccctccttagcatcttat

SEQ ID NO: 1072 - DNA, 21 bp, synthetic construct
  misc_feature 1..21: note = oligonucleotide
  source 1..21: mol_type = other DNA
aggaagagaatctccgcaaga

SEQ ID NO: 1073 - DNA, 21 bp, synthetic construct
  misc_feature 1..21: note = oligonucleotide
  source 1..21: mol_type = other DNA
gcttgtgagctttactgagaa

SEQ ID NO: 1074 - DNA, 21 bp, synthetic construct
  misc_feature 1..21: note = oligonucleotide
  source 1..21: mol_type = other DNA
agaaagagaatctccgcaaga

SEQ ID NO: 1075 - DNA, 21 bp, synthetic construct
  misc_feature 1..21: note = oligonucleotide
  source 1..21: mol_type = other DNA
gaagagaatctccgcaagaaa

SEQ ID NO: 1076 - DNA, 21 bp, synthetic construct
  misc_feature 1..21: note = oligonucleotide
  source 1..21: mol_type = other DNA
ccggtggctgggccactctgc

SEQ ID NO: 1077 - DNA, 21 bp, synthetic construct
  misc_feature 1..21: note = oligonucleotide
  source 1..21: mol_type = other DNA
agtgcaatgagggaccagtac

SEQ ID NO: 1078 - DNA, 21 bp, synthetic construct
  misc_feature 1..21: note = oligonucleotide
  source 1..21: mol_type = other DNA
ccatcctcaccatcatcacac

SEQ ID NO: 1079 - DNA, 21 bp, synthetic construct
  misc_feature 1..21: note = oligonucleotide
  source 1..21: mol_type = other DNA
ggccagacctaagagcaatca

SEQ ID NO: 1080 - DNA, 21 bp, synthetic construct
  misc_feature 1..21: note = oligonucleotide
  source 1..21: mol_type = other DNA
tcccgtcatgtgggtcctgaa

SEQ ID NO: 1081 - DNA, 21 bp, synthetic construct
  misc_feature 1..21: note = oligonucleotide
  source 1..21: mol_type = other DNA
aggaagagaatctccgcaaga

SEQ ID NO: 1082 - DNA, 21 bp, synthetic construct
  misc_feature 1..21: note = oligonucleotide
  source 1..21: mol_type = other DNA
tggcgtggacgcgggtgccgg

SEQ ID NO: 1083 - DNA, 21 bp, synthetic construct
  misc_feature 1..21: note = oligonucleotide
  source 1..21: mol_type = other DNA
ggccagacctaagagcaatca

SEQ ID NO: 1084 - DNA, 21 bp, synthetic construct
  misc_feature 1..21: note = oligonucleotide
  source 1..21: mol_type = other DNA
ggaagagaggtggcgtggcct

SEQ ID NO: 1085 - DNA, 21 bp, synthetic construct
  misc_feature 1..21: note = oligonucleotide
  source 1..21: mol_type = other DNA
tggcgaggacgcgggtgccgg

SEQ ID NO: 1086 - DNA, 21 bp, synthetic construct
  misc_feature 1..21: note = oligonucleotide
  source 1..21: mol_type = other DNA
caagatgccactcttagggtt

SEQ ID NO: 1087 - DNA, 21 bp, synthetic construct
  misc_feature 1..21: note = oligonucleotide
  source 1..21: mol_type = other DNA
tggcgcggaagcgggtgccgg

SEQ ID NO: 1088 - DNA, 21 bp, synthetic construct
  misc_feature 1..21: note = oligonucleotide
  source 1..21: mol_type = other DNA
gtgtcatctgagcacaggttc

SEQ ID NO: 1089 - DNA, 21 bp, synthetic construct
  misc_feature 1..21: note = oligonucleotide
  source 1..21: mol_type = other DNA
aggaagagaatctccgcaaga

SEQ ID NO: 1090 - DNA, 21 bp, synthetic construct
  misc_feature 1..21: note = oligonucleotide
  source 1..21: mol_type = other DNA
ctccggacccaccatctaggg

SEQ ID NO: 1091 - DNA, 21 bp, synthetic construct
  misc_feature 1..21: note = oligonucleotide
  source 1..21: mol_type = other DNA
ctccggacccaccatttgggg

SEQ ID NO: 1092 - DNA, 21 bp, synthetic construct
  misc_feature 1..21: note = oligonucleotide
  source 1..21: mol_type = other DNA
ggaagagaggtggcgtgacct

SEQ ID NO: 1093 - DNA, 21 bp, synthetic construct
  misc_feature 1..21: note = oligonucleotide
  source 1..21: mol_type = other DNA
taaactgtgaactcctttagg

SEQ ID NO: 1094 - DNA, 21 bp, synthetic construct
  misc_feature 1..21: note = oligonucleotide
  source 1..21: mol_type = other DNA
gaggctgcggcccaccgcacc

SEQ ID NO: 1095 - DNA, 21 bp, synthetic construct
  misc_feature 1..21: note = oligonucleotide
  source 1..21: mol_type = other DNA
acgcccacggatctgcagcaa

SEQ ID NO: 1096 - DNA, 21 bp, synthetic construct
  misc_feature 1..21: note = oligonucleotide
  source 1..21: mol_type = other DNA
tcacagcacatgacggaggtt

SEQ ID NO: 1097 - DNA, 21 bp, synthetic construct
  misc_feature 1..21: note = oligonucleotide
  source 1..21: mol_type = other DNA
ggaaatttgcgtgtggagtat

SEQ ID NO: 1098 - DNA, 21 bp, synthetic construct
  misc_feature 1..21: note = oligonucleotide
  source 1..21: mol_type = other DNA
atgaacttgtcttcccgtcat

SEQ ID NO: 1099 - DNA, 21 bp, synthetic construct
  misc_feature 1..21: note = oligonucleotide
  source 1..21: mol_type = other DNA
cttgtccggctcatacggagt

SEQ ID NO: 1100 - DNA, 21 bp, synthetic construct
  misc_feature 1..21: note = oligonucleotide
  source 1..21: mol_type = other DNA
tatctgagcagcgctcatggt

SEQ ID NO: 1101 - DNA, 21 bp, synthetic construct
  misc_feature 1..21: note = oligonucleotide
  source 1..21: mol_type = other DNA
gtgtcatctgagcacaggttc

SEQ ID NO: 1102 - DNA, 21 bp, synthetic construct
  misc_feature 1..21: note = oligonucleotide
  source 1..21: mol_type = other DNA
tcacagcacatgacggaggtt

SEQ ID NO: 1103 - DNA, 21 bp, synthetic construct
  misc_feature 1..21: note = oligonucleotide
  source 1..21: mol_type = other DNA
cggtgggccgcagcctccaag

SEQ ID NO: 1104 - DNA, 21 bp, synthetic construct
  misc_feature 1..21: note = oligonucleotide
  source 1..21: mol_type = other DNA
ggaagaggggtggcgtgaccg

SEQ ID NO: 1105 - DNA, 21 bp, synthetic construct
  misc_feature 1..21: note = oligonucleotide
  source 1..21: mol_type = other DNA
ggtaaaggcaatcctgaggaa

SEQ ID NO: 1106 - DNA, 21 bp, synthetic construct
  misc_feature 1..21: note = oligonucleotide
  source 1..21: mol_type = other DNA
ggaaatttgcgtgtggagtat

SEQ ID NO: 1107 - DNA, 21 bp, synthetic construct
  misc_feature 1..21: note = oligonucleotide
  source 1..21: mol_type = other DNA
ggaaatttgcgtgtggagtgt

SEQ ID NO: 1108 - DNA, 21 bp, synthetic construct
  misc_feature 1..21: note = oligonucleotide
  source 1..21: mol_type = other DNA
ggccagacctaagagcaatca

SEQ ID NO: 1109 - DNA, 21 bp, synthetic construct
  misc_feature 1..21: note = oligonucleotide
  source 1..21: mol_type = other DNA
acccacccccaaatggtgggt

SEQ ID NO: 1110 - DNA, 21 bp, synthetic construct
  misc_feature 1..21: note = oligonucleotide
  source 1..21: mol_type = other DNA
ggaaatttgcgtgtggagtat

SEQ ID NO: 1111 - DNA, 21 bp, synthetic construct
  misc_feature 1..21: note = oligonucleotide
  source 1..21: mol_type = other DNA
acccacccctagatggtgggt

SEQ ID NO: 1112 - DNA, 21 bp, synthetic construct
  misc_feature 1..21: note = oligonucleotide
  source 1..21: mol_type = other DNA
aggccagggtcctgggcgctc

SEQ ID NO: 1113 - DNA, 21 bp, synthetic construct
  misc_feature 1..21: note = oligonucleotide
  source 1..21: mol_type = other DNA
ggaaatttgcgtgtggagtat

SEQ ID NO: 1114 - DNA, 21 bp, synthetic construct
  misc_feature 1..21: note = oligonucleotide
  source 1..21: mol_type = other DNA
agccatattcctgcaggcgct

SEQ ID NO: 1115 - DNA, 21 bp, synthetic construct
  misc_feature 1..21: note = oligonucleotide
  source 1..21: mol_type = other DNA
tacaagcagtcacagcacatg

SEQ ID NO: 1116 - DNA, 21 bp, synthetic construct
  misc_feature 1..21: note = oligonucleotide
  source 1..21: mol_type = other DNA
tgtggggggcctgcggtcgga

SEQ ID NO: 1117 - DNA, 21 bp, synthetic construct
  misc_feature 1..21: note = oligonucleotide
  source 1..21: mol_type = other DNA
cggcgggccgcagcctccaag

SEQ ID NO: 1118 - DNA, 21 bp, synthetic construct
  misc_feature 1..21: note = oligonucleotide
  source 1..21: mol_type = other DNA
ggtgggccgcagcctccaagc

SEQ ID NO: 1119 - DNA, 21 bp, synthetic construct
  misc_feature 1..21: note = oligonucleotide
  source 1..21: mol_type = other DNA
cgagggacccaggtctacgat

SEQ ID NO: 1120 - DNA, 21 bp, synthetic construct
  misc_feature 1..21: note = oligonucleotide
  source 1..21: mol_type = other DNA
tacaagcagtcacagcacatg

SEQ ID NO: 1121 - DNA, 21 bp, synthetic construct
  misc_feature 1..21: note = oligonucleotide
  source 1..21: mol_type = other DNA
ggaagagcggtggcctgacct

SEQ ID NO: 1122 - DNA, 21 bp, synthetic construct
  misc_feature 1..21: note = oligonucleotide
  source 1..21: mol_type = other DNA
tggcgcggacgcgggtgccgg

SEQ ID NO: 1123 - DNA, 21 bp, synthetic construct
  misc_feature 1..21: note = oligonucleotide
  source 1..21: mol_type = other DNA
ccggtggctgggccactctgc

SEQ ID NO: 1124 - DNA, 21 bp, synthetic construct
  misc_feature 1..21: note = oligonucleotide
  source 1..21: mol_type = other DNA
gggcgggcgcgtggggccttg

SEQ ID NO: 1125 - DNA, 21 bp, synthetic construct
  misc_feature 1..21: note = oligonucleotide
  source 1..21: mol_type = other DNA
ggaagagaggtggcgtgacct

SEQ ID NO: 1126 - DNA, 21 bp, synthetic construct
  misc_feature 1..21: note = oligonucleotide
  source 1..21: mol_type = other DNA
agcgtcgtgcacgggtcaggt

SEQ ID NO: 1127 - DNA, 21 bp, synthetic construct
  misc_feature 1..21: note = oligonucleotide
  source 1..21: mol_type = other DNA
acaagcagtcacagcacatga

SEQ ID NO: 1128 - DNA, 21 bp, synthetic construct
  misc_feature 1..21: note = oligonucleotide
  source 1..21: mol_type = other DNA
gacttgtccggctcatacgga

SEQ ID NO: 1129 - DNA, 21 bp, synthetic construct
  misc_feature 1..21: note = oligonucleotide
  source 1..21: mol_type = other DNA
acaagcagtcacagcacatga

SEQ ID NO: 1130 - DNA, 21 bp, synthetic construct
  misc_feature 1..21: note = oligonucleotide
  source 1..21: mol_type = other DNA
aactgctgggtgcggaagaga

SEQ ID NO: 1131 - DNA, 21 bp, synthetic construct
  misc_feature 1..21: note = oligonucleotide
  source 1..21: mol_type = other DNA
gtcaccgtagtggtggtggtg

SEQ ID NO: 1132 - DNA, 21 bp, synthetic construct
  misc_feature 1..21: note = oligonucleotide
  source 1..21: mol_type = other DNA
ggcgggccgcagcctccaagc

SEQ ID NO: 1133 - DNA, 21 bp, synthetic construct
  misc_feature 1..21: note = oligonucleotide
  source 1..21: mol_type = other DNA
ttgtccggctcatacggagtg

SEQ ID NO: 1134 - DNA, 21 bp, synthetic construct
  misc_feature 1..21: note = oligonucleotide
  source 1..21: mol_type = other DNA
ctgggggagtttgccaaggtg

SEQ ID NO: 1135 - DNA, 21 bp, synthetic construct
  misc_feature 1..21: note = oligonucleotide
  source 1..21: mol_type = other DNA
agagatatgaggaaatcgtta

SEQ ID NO: 1136 - DNA, 21 bp, synthetic construct
  misc_feature 1..21: note = oligonucleotide
  source 1..21: mol_type = other DNA
gtgggcattcccacgcatgtt

SEQ ID NO: 1137 - DNA, 21 bp, synthetic construct
  misc_feature 1..21: note = oligonucleotide
  source 1..21: mol_type = other DNA
tggcccagggacagctcagtg

SEQ ID NO: 1138 - DNA, 21 bp, synthetic construct
  misc_feature 1..21: note = oligonucleotide
  source 1..21: mol_type = other DNA
aggttgggcaatggtagagta

SEQ ID NO: 1139 - DNA, 21 bp, synthetic construct
  misc_feature 1..21: note = oligonucleotide
  source 1..21: mol_type = other DNA
tgcttgtagatggccatggcg

SEQ ID NO: 1140 - DNA, 21 bp, synthetic construct
  misc_feature 1..21: note = oligonucleotide
  source 1..21: mol_type = other DNA
gggtcctgggcgctcgtgaag

SEQ ID NO: 1141 - DNA, 21 bp, synthetic construct
  misc_feature 1..21: note = oligonucleotide
  source 1..21: mol_type = other DNA
gattttgggcgggccaaactg

SEQ ID NO: 1142 - DNA, 21 bp, synthetic construct
  misc_feature 1..21: note = oligonucleotide
  source 1..21: mol_type = other DNA
tgcaagcagtcacagcacatg

SEQ ID NO: 1143 - DNA, 21 bp, synthetic construct
  misc_feature 1..21: note = oligonucleotide
  source 1..21: mol_type = other DNA
gcccttttttgggttttcggc

SEQ ID NO: 1144 - DNA, 21 bp, synthetic construct
  misc_feature 1..21: note = oligonucleotide
  source 1..21: mol_type = other DNA
tgttgttgggcagtgctagga

SEQ ID NO: 1145 - DNA, 21 bp, synthetic construct
  misc_feature 1..21: note = oligonucleotide
  source 1..21: mol_type = other DNA
cccccatgtcgcctctgtagg

SEQ ID NO: 1146 - DNA, 21 bp, synthetic construct
  misc_feature 1..21: note = oligonucleotide
  source 1..21: mol_type = other DNA
agtgcggcggccttgccggcg

SEQ ID NO: 1147 - DNA, 21 bp, synthetic construct
  misc_feature 1..21: note = oligonucleotide
  source 1..21: mol_type = other DNA
catgagctcttaccttgtcac

SEQ ID NO: 1148 - DNA, 21 bp, synthetic construct
  misc_feature 1..21: note = oligonucleotide
  source 1..21: mol_type = other DNA
gtcctgggagagaccggcgca

SEQ ID NO: 1149 - DNA, 21 bp, synthetic construct
  misc_feature 1..21: note = oligonucleotide
  source 1..21: mol_type = other DNA
ctgaatgaggccttggaactc

SEQ ID NO: 1150 - DNA, 21 bp, synthetic construct
  misc_feature 1..21: note = oligonucleotide
  source 1..21: mol_type = other DNA
gcaagcagtcacagcacatga

SEQ ID NO: 1151 - DNA, 21 bp, synthetic construct
  misc_feature 1..21: note = oligonucleotide
  source 1..21: mol_type = other DNA
ggcaaaacatcttgttgaggg

SEQ ID NO: 1152 - DNA, 21 bp, synthetic construct
  misc_feature 1..21: note = oligonucleotide
  source 1..21: mol_type = other DNA
gtcctgggagagaccggcgca

SEQ ID NO: 1153 - DNA, 21 bp, synthetic construct
  misc_feature 1..21: note = oligonucleotide
  source 1..21: mol_type = other DNA
agacctgcccggcagtagtca

SEQ ID NO: 1154 - DNA, 21 bp, synthetic construct
  misc_feature 1..21: note = oligonucleotide
  source 1..21: mol_type = other DNA
gtcctgggacagaccggcgca

SEQ ID NO: 1155 - DNA, 21 bp, synthetic construct
  misc_feature 1..21: note = oligonucleotide
  source 1..21: mol_type = other DNA
gtcctgggagagaccggcgca

SEQ ID NO: 1156 - DNA, 21 bp, synthetic construct
  misc_feature 1..21: note = oligonucleotide
  source 1..21: mol_type = other DNA
gtcctgggagagactggcgca

SEQ ID NO: 1157 - DNA, 21 bp, synthetic construct
  misc_feature 1..21: note = oligonucleotide
  source 1..21: mol_type = other DNA
gtggtaaaggcaatcctgagg

SEQ ID NO: 1158 - DNA, 21 bp, synthetic construct
  misc_feature 1..21: note = oligonucleotide
  source 1..21: mol_type = other DNA
gtcaccgtggtggtggtggta

SEQ ID NO: 1159 - DNA, 21 bp, synthetic construct
  misc_feature 1..21: note = oligonucleotide
  source 1..21: mol_type = other DNA
gtcaccgtggtggtggtggtg

SEQ ID NO: 1160 - DNA, 21 bp, synthetic construct
  misc_feature 1..21: note = oligonucleotide
  source 1..21: mol_type = other DNA
tccagaagtcaggacacagca

SEQ ID NO: 1161 - DNA, 21 bp, synthetic construct
  misc_feature 1..21: note = oligonucleotide
  source 1..21: mol_type = other DNA
gtcctgggaaagaccggcgca

SEQ ID NO: 1162 - DNA, 21 bp, synthetic construct
  misc_feature 1..21: note = oligonucleotide
  source 1..21: mol_type = other DNA
gtcctgggagagaccggcgca

SEQ ID NO: 1163 - DNA, 21 bp, synthetic construct
  misc_feature 1..21: note = oligonucleotide
  source 1..21: mol_type = other DNA
tgagtggtaaaggcaatcctg

SEQ ID NO: 1164 - DNA, 21 bp, synthetic construct
  misc_feature 1..21: note = oligonucleotide
  source 1..21: mol_type = other DNA
actcatttcctctccctccgc

SEQ ID NO: 1165 - DNA, 21 bp, synthetic construct
  misc_feature 1..21: note = oligonucleotide
  source 1..21: mol_type = other DNA
agccatattcctgcaggcgct

SEQ ID NO: 1166 - DNA, 21 bp, synthetic construct
  misc_feature 1..21: note = oligonucleotide
  source 1..21: mol_type = other DNA
gccatcgctctggtggagaga

SEQ ID NO: 1167 - DNA, 21 bp, synthetic construct
  misc_feature 1..21: note = oligonucleotide
  source 1..21: mol_type = other DNA
gcacaggttccggaagtacct

SEQ ID NO: 1168 - DNA, 21 bp, synthetic construct
  misc_feature 1..21: note = oligonucleotide
  source 1..21: mol_type = other DNA
tgttgaggaagggctggtgac

SEQ ID NO: 1169 - DNA, 21 bp, synthetic construct
  misc_feature 1..21: note = oligonucleotide
  source 1..21: mol_type = other DNA
ggcggccttgccggcgtcgga

SEQ ID NO: 1170 - DNA, 21 bp, synthetic construct
  misc_feature 1..21: note = oligonucleotide
  source 1..21: mol_type = other DNA
catgcggaggttgggcaatgg

SEQ ID NO: 1171 - DNA, 21 bp, synthetic construct
  misc_feature 1..21: note = oligonucleotide
  source 1..21: mol_type = other DNA
aggaagagaatctccgcaaga

SEQ ID NO: 1172 - DNA, 21 bp, synthetic construct
  misc_feature 1..21: note = oligonucleotide
  source 1..21: mol_type = other DNA
gaagagaatctccgcaagaaa

SEQ ID NO: 1173 - DNA, 21 bp, synthetic construct
  misc_feature 1..21: note = oligonucleotide
  source 1..21: mol_type = other DNA
tcacactggaagactccaggt

SEQ ID NO: 1174 - DNA, 21 bp, synthetic construct
  misc_feature 1..21: note = oligonucleotide
  source 1..21: mol_type = other DNA
agaaagagaatctccgcaaga

SEQ ID NO: 1175 - DNA, 21 bp, synthetic construct
  misc_feature 1..21: note = oligonucleotide
  source 1..21: mol_type = other DNA
agctgctgtgggccaccgctg

SEQ ID NO: 1176 - DNA, 21 bp, synthetic construct
  misc_feature 1..21: note = oligonucleotide
  source 1..21: mol_type = other DNA
ccatacaggttctcggtggtg

SEQ ID NO: 1177 - DNA, 21 bp, synthetic construct
  misc_feature 1..21: note = oligonucleotide
  source 1..21: mol_type = other DNA
aaagagaatctccgcaagaaa

SEQ ID NO: 1178 - DNA, 21 bp, synthetic construct
  misc_feature 1..21: note = oligonucleotide
  source 1..21: mol_type = other DNA
gcggccttgccggcgtcggag

SEQ ID NO: 1179 - DNA, 21 bp, synthetic construct
  misc_feature 1..21: note = oligonucleotide
  source 1..21: mol_type = other DNA
aggaagagaatctccgcaaga

SEQ ID NO: 1180 - DNA, 21 bp, synthetic construct
  misc_feature 1..21: note = oligonucleotide
  source 1..21: mol_type = other DNA
caagaagcggtggaggagacc

SEQ ID NO: 1181 - DNA, 21 bp, synthetic construct
  misc_feature 1..21: note = oligonucleotide
  source 1..21: mol_type = other DNA
taagagcaatcagtgaggaat

SEQ ID NO: 1182 - DNA, 21 bp, synthetic construct
  misc_feature 1..21: note = oligonucleotide
  source 1..21: mol_type = other DNA
gaagagaatctccgcaagaaa

SEQ ID NO: 1183 - DNA, 21 bp, synthetic construct
  misc_feature 1..21: note = oligonucleotide
  source 1..21: mol_type = other DNA
tagtggcaccaaaatggattc

SEQ ID NO: 1184 - DNA, 21 bp, synthetic construct
  misc_feature 1..21: note = oligonucleotide
  source 1..21: mol_type = other DNA
tagtggcaccagaatggattc

SEQ ID NO: 1185 - DNA, 21 bp, synthetic construct
  misc_feature 1..21: note = oligonucleotide
  source 1..21: mol_type = other DNA
tcctccaggtagctcatcccc

SEQ ID NO: 1186 - DNA, 21 bp, synthetic construct
  misc_feature 1..21: note = oligonucleotide
  source 1..21: mol_type = other DNA
aagcaagcaggacaagaagcg

SEQ ID NO: 1187 - DNA, 21 bp, synthetic construct
  misc_feature 1..21: note = oligonucleotide
  source 1..21: mol_type = other DNA
cagtggcaccagaatggattc

SEQ ID NO: 1188 - DNA, 21 bp, synthetic construct
  misc_feature 1..21: note = oligonucleotide
  source 1..21: mol_type = other DNA
gaggagaccggaaaatgggag

SEQ ID NO: 1189 - DNA, 21 bp, synthetic construct
  misc_feature 1..21: note = oligonucleotide
  source 1..21: mol_type = other DNA
gaaggggagtgagcagacaca

SEQ ID NO: 1190 - DNA, 21 bp, synthetic construct
  misc_feature 1..21: note = oligonucleotide
  source 1..21: mol_type = other DNA
agtgcggcggccttgccggcg

SEQ ID NO: 1191 - DNA, 21 bp, synthetic construct
  misc_feature 1..21: note = oligonucleotide
  source 1..21: mol_type = other DNA
aaattgagtgggttctaatca

SEQ ID NO: 1192 - DNA, 21 bp, synthetic construct
  misc_feature 1..21: note = oligonucleotide
  source 1..21: mol_type = other DNA
ggaaggggagtgagcagacac

SEQ ID NO: 1193 - DNA, 21 bp, synthetic construct
  misc_feature 1..21: note = oligonucleotide
  source 1..21: mol_type = other DNA
gatgcgtgtttgtgcctgtcc

SEQ ID NO: 1194 - DNA, 21 bp, synthetic construct
  misc_feature 1..21: note = oligonucleotide
  source 1..21: mol_type = other DNA
ggtgtgtgtttgtgcctgtcc

SEQ ID NO: 1195 - DNA, 21 bp, synthetic construct
  misc_feature 1..21: note = oligonucleotide
  source 1..21: mol_type = other DNA
ggtgcttgtttgtgcctgtcc

SEQ ID NO: 1196 - DNA, 21 bp, synthetic construct
  misc_feature 1..21: note = oligonucleotide
  source 1..21: mol_type = other DNA
ggtgcatgtttgtgcctgtcc

SEQ ID NO: 1197 - DNA, 21 bp, synthetic construct
  misc_feature 1..21: note = oligonucleotide
  source 1..21: mol_type = other DNA
ggtgcgtgtttatgcctgtcc

SEQ ID NO: 1198 - DNA, 21 bp, synthetic construct
  misc_feature 1..21: note = oligonucleotide
  source 1..21: mol_type = other DNA
ggaagtgtcggtgacaggaag

SEQ ID NO: 1199 - DNA, 21 bp, synthetic construct
  misc_feature 1..21: note = oligonucleotide
  source 1..21: mol_type = other DNA
gagagagggaagaaaggagga

SEQ ID NO: 1200 - DNA, 21 bp, synthetic construct
  misc_feature 1..21: note = oligonucleotide
  source 1..21: mol_type = other DNA
ggaaggggagtgagcagacac

SEQ ID NO: 1201 - DNA, 21 bp, synthetic construct
  misc_feature 1..21: note = oligonucleotide
  source 1..21: mol_type = other DNA
gaaggggagtgagcagacaca

SEQ ID NO: 1202 - DNA, 21 bp, synthetic construct
  misc_feature 1..21: note = oligonucleotide
  source 1..21: mol_type = other DNA
ctcctgcgcagtgatttcaga

SEQ ID NO: 1203 - DNA, 21 bp, synthetic construct
  misc_feature 1..21: note = oligonucleotide
  source 1..21: mol_type = other DNA
agcaagcaggacaagaagcgg

SEQ ID NO: 1204 - DNA, 21 bp, synthetic construct
  misc_feature 1..21: note = oligonucleotide
  source 1..21: mol_type = other DNA
ctcctgcttagtgatttcaga

SEQ ID NO: 1205 - DNA, 21 bp, synthetic construct
  misc_feature 1..21: note = oligonucleotide
  source 1..21: mol_type = other DNA
caagcaggacaagaagcggtg

SEQ ID NO: 1206 - DNA, 21 bp, synthetic construct
  misc_feature 1..21: note = oligonucleotide
  source 1..21: mol_type = other DNA
gacccgtgcacgacgctgccc

SEQ ID NO: 1207 - DNA, 21 bp, synthetic construct
  misc_feature 1..21: note = oligonucleotide
  source 1..21: mol_type = other DNA
ttgacttcagtcagcgacgga

SEQ ID NO: 1208 - DNA, 21 bp, synthetic construct
  misc_feature 1..21: note = oligonucleotide
  source 1..21: mol_type = other DNA
aagcaagcaggacaagaagcg

SEQ ID NO: 1209 - DNA, 21 bp, synthetic construct
  misc_feature 1..21: note = oligonucleotide
  source 1..21: mol_type = other DNA
ctcccgctcagtgatttcaga

SEQ ID NO: 1210 - DNA, 21 bp, synthetic construct
  misc_feature 1..21: note = oligonucleotide
  source 1..21: mol_type = other DNA
ctcctgctcagtgattttaga

SEQ ID NO: 1211 - DNA, 21 bp, synthetic construct
  misc_feature 1..21: note = oligonucleotide
  source 1..21: mol_type = other DNA
gtcctgggagagaccggcgca

SEQ ID NO: 1212 - DNA, 21 bp, synthetic construct
  misc_feature 1..21: note = oligonucleotide
  source 1..21: mol_type = other DNA
agcaagcaggacaagaagcgg

SEQ ID NO: 1213 - DNA, 21 bp, synthetic construct
  misc_feature 1..21: note = oligonucleotide
  source 1..21: mol_type = other DNA
ggagctccctggactggagcc

SEQ ID NO: 1214 - DNA, 21 bp, synthetic construct
  misc_feature 1..21: note = oligonucleotide
  source 1..21: mol_type = other DNA
gtggatgtggggggcctgcgg

SEQ ID NO: 1215 - DNA, 21 bp, synthetic construct
  misc_feature 1..21: note = oligonucleotide
  source 1..21: mol_type = other DNA
agagatatgaggaaatcgtta

SEQ ID NO: 1216 - DNA, 21 bp, synthetic construct
  misc_feature 1..21: note = oligonucleotide
  source 1..21: mol_type = other DNA
acttgcactgtcagttgccct

SEQ ID NO: 1217 - DNA, 21 bp, synthetic construct
  misc_feature 1..21: note = oligonucleotide
  source 1..21: mol_type = other DNA
gggagctccctggactggagc

SEQ ID NO: 1218 - DNA, 21 bp, synthetic construct
  misc_feature 1..21: note = oligonucleotide
  source 1..21: mol_type = other DNA
tatcatccgctgagggtggaa

SEQ ID NO: 1219 - DNA, 21 bp, synthetic construct
  misc_feature 1..21: note = oligonucleotide
  source 1..21: mol_type = other DNA
tgacccgtgcacgacgctgcc

SEQ ID NO: 1220 - DNA, 21 bp, synthetic construct
  misc_feature 1..21: note = oligonucleotide
  source 1..21: mol_type = other DNA
gacaaaaaaaaaaggtttcaa

SEQ ID NO: 1221 - DNA, 21 bp, synthetic construct
  misc_feature 1..21: note = oligonucleotide
  source 1..21: mol_type = other DNA
gagtaagaaaaaaaaaagaat

SEQ ID NO: 1222 - DNA, 21 bp, synthetic construct
  misc_feature 1..21: note = oligonucleotide
  source 1..21: mol_type = other DNA
tttaaaaaaaaaaagaaaaaa

SEQ ID NO: 1223 - DNA, 21 bp, synthetic construct
  misc_feature 1..21: note = oligonucleotide
  source 1..21: mol_type = other DNA
tggagtaagaaaaaaaaaaga

SEQ ID NO: 1224 - DNA, 21 bp, synthetic construct
  misc_feature 1..21: note = oligonucleotide
  source 1..21: mol_type = other DNA
ctgctacttttttttcaaaac

SEQ ID NO: 1225 - DNA, 21 bp, synthetic construct
  misc_feature 1..21: note = oligonucleotide
  source 1..21: mol_type = other DNA
tggggcagtgggcatgttatg

SEQ ID NO: 1226 - DNA, 21 bp, synthetic construct
  misc_feature 1..21: note = oligonucleotide
  source 1..21: mol_type = other DNA
taaaaaaaaatacttgtaggc

SEQ ID NO: 1227 - DNA, 21 bp, synthetic construct
  misc_feature 1..21: note = oligonucleotide
  source 1..21: mol_type = other DNA
gggcagtgggcatgttatggg

SEQ ID NO: 1228 - DNA, 21 bp, synthetic construct
  misc_feature 1..21: note = oligonucleotide
  source 1..21: mol_type = other DNA
tctttaaatcagtagtctaac

SEQ ID NO: 1229 - DNA, 21 bp, synthetic construct
  misc_feature 1..21: note = oligonucleotide
  source 1..21: mol_type = other DNA
aaaatgcatatgcaatgtccc

SEQ ID NO: 1230 - DNA, 21 bp, synthetic construct
  misc_feature 1..21: note = oligonucleotide
  source 1..21: mol_type = other DNA
agaaaaaaaaaagtcatcaat

SEQ ID NO: 1231 - DNA, 21 bp, synthetic construct
  misc_feature 1..21: note = oligonucleotide
  source 1..21: mol_type = other DNA
cttattattatagtgccgccc

SEQ ID NO: 1232 - DNA, 21 bp, synthetic construct
  misc_feature 1..21: note = oligonucleotide
  source 1..21: mol_type = other DNA
ttggggcagtgggcatgttat

SEQ ID NO: 1233 - DNA, 21 bp, synthetic construct
  misc_feature 1..21: note = oligonucleotide
  source 1..21: mol_type = other DNA
atgactttttttttttttaac

SEQ ID NO: 1234 - DNA, 21 bp, synthetic construct
  misc_feature 1..21: note = oligonucleotide
  source 1..21: mol_type = other DNA
ggaaatctttaaaaaaaaatt

SEQ ID NO: 1235 - DNA, 21 bp, synthetic construct
  misc_feature 1..21: note = oligonucleotide
  source 1..21: mol_type = other DNA
tctgtctgagaaaaaaaaaaa

SEQ ID NO: 1236 - DNA, 21 bp, synthetic construct
  misc_feature 1..21: note = oligonucleotide
  source 1..21: mol_type = other DNA
aagaggaggaaaaggaaaaaa

SEQ ID NO: 1237 - DNA, 21 bp, synthetic construct
  misc_feature 1..21: note = oligonucleotide
  source 1..21: mol_type = other DNA
tttaagagtggaagaaaaaaa

SEQ ID NO: 1238 - DNA, 21 bp, synthetic construct
  misc_feature 1..21: note = oligonucleotide
  source 1..21: mol_type = other DNA
gttaaggaaaaagaggaaaag

SEQ ID NO: 1239 - DNA, 21 bp, synthetic construct
  misc_feature 1..21: note = oligonucleotide
  source 1..21: mol_type = other DNA
agcagagctggggggtgcaac

SEQ ID NO: 1240 - DNA, 21 bp, synthetic construct
  misc_feature 1..21: note = oligonucleotide
  source 1..21: mol_type = other DNA
cacacccccttcagccgcctg

SEQ ID NO: 1241 - DNA, 21 bp, synthetic construct
  misc_feature 1..21: note = oligonucleotide
  source 1..21: mol_type = other DNA
agtgtatcagactccaaaaaa

SEQ ID NO: 1242 - DNA, 21 bp, synthetic construct
  misc_feature 1..21: note = oligonucleotide
  source 1..21: mol_type = other DNA
tctctctcttttttttttttt

SEQ ID NO: 1243 - DNA, 21 bp, synthetic construct
  misc_feature 1..21: note = oligonucleotide
  source 1..21: mol_type = other DNA
ctctctttttttttttttgag

SEQ ID NO: 1244 - DNA, 21 bp, synthetic construct
  misc_feature 1..21: note = oligonucleotide
  source 1..21: mol_type = other DNA
atttggttgagatctggaggg

SEQ ID NO: 1245 - DNA, 21 bp, synthetic construct
  misc_feature 1..21: note = oligonucleotide
  source 1..21: mol_type = other DNA
cacatttttttttttaaagaa

SEQ ID NO: 1246 - DNA, 21 bp, synthetic construct
  misc_feature 1..21: note = oligonucleotide
  source 1..21: mol_type = other DNA
aaatgaaagtgatgatgatga

SEQ ID NO: 1247 - DNA, 21 bp, synthetic construct
  misc_feature 1..21: note = oligonucleotide
  source 1..21: mol_type = other DNA
tttttctttttttttttcttg

SEQ ID NO: 1248 - DNA, 21 bp, synthetic construct
  misc_feature 1..21: note = oligonucleotide
  source 1..21: mol_type = other DNA
tttttttaattactgggtttt

SEQ ID NO: 1249 - DNA, 21 bp, synthetic construct
  misc_feature 1..21: note = oligonucleotide
  source 1..21: mol_type = other DNA
tgatttttttttcctaattgt

SEQ ID NO: 1250 - DNA, 21 bp, synthetic construct
  misc_feature 1..21: note = oligonucleotide
  source 1..21: mol_type = other DNA
aacagtctttttttttaatga

SEQ ID NO: 1251 - DNA, 21 bp, synthetic construct
  misc_feature 1..21: note = oligonucleotide
  source 1..21: mol_type = other DNA
aaatactgatgttcctagtga

SEQ ID NO: 1252 - DNA, 21 bp, synthetic construct
  misc_feature 1..21: note = oligonucleotide
  source 1..21: mol_type = other DNA
aaatggacctttttttttcta

SEQ ID NO: 1253 - DNA, 21 bp, synthetic construct
  misc_feature 1..21: note = oligonucleotide
  source 1..21: mol_type = other DNA
ttggaaatctttaaaaaaaaa

SEQ ID NO: 1254 - DNA, 21 bp, synthetic construct
  misc_feature 1..21: note = oligonucleotide
  source 1..21: mol_type = other DNA
gcagagctggggggtgcaacc

SEQ ID NO: 1255 - DNA, 21 bp, synthetic construct
  misc_feature 1..21: note = oligonucleotide
  source 1..21: mol_type = other DNA
tttgttttttttttaaatatt

SEQ ID NO: 1256 - DNA, 21 bp, synthetic construct
  misc_feature 1..21: note = oligonucleotide
  source 1..21: mol_type = other DNA
tgagatttttttttttctgaa

SEQ ID NO: 1257 - DNA, 21 bp, synthetic construct
  misc_feature 1..21: note = oligonucleotide
  source 1..21: mol_type = other DNA
tcaaaaaatactcattgcgca

SEQ ID NO: 1258 - DNA, 21 bp, synthetic construct
  misc_feature 1..21: note = oligonucleotide
  source 1..21: mol_type = other DNA
ttgagcttttggggaaaaaaa

SEQ ID NO: 1259 - DNA, 21 bp, synthetic construct
  misc_feature 1..21: note = oligonucleotide
  source 1..21: mol_type = other DNA
ttctatactttttttttgagg

SEQ ID NO: 1260 - DNA, 21 bp, synthetic construct
  misc_feature 1..21: note = oligonucleotide
  source 1..21: mol_type = other DNA
gatgcttttttttcccccagt

SEQ ID NO: 1261 - DNA, 21 bp, synthetic construct
  misc_feature 1..21: note = oligonucleotide
  source 1..21: mol_type = other DNA
gagatgcttttttttccccca

SEQ ID NO: 1262 - DNA, 21 bp, synthetic construct
  misc_feature 1..21: note = oligonucleotide
  source 1..21: mol_type = other DNA
tctaagcatttcttaactgag

SEQ ID NO: 1263 - DNA, 21 bp, synthetic construct
  misc_feature 1..21: note = oligonucleotide
  source 1..21: mol_type = other DNA
ctggcctggaggctatccagc

SEQ ID NO: 1264 - DNA, 21 bp, synthetic construct
  misc_feature 1..21: note = oligonucleotide
  source 1..21: mol_type = other DNA
atctaagcatttcttaactga

SEQ ID NO: 1265 - DNA, 21 bp, synthetic construct
  misc_feature 1..21: note = oligonucleotide
  source 1..21: mol_type = other DNA
cagacggcttttttcgccaac

SEQ ID NO: 1266 - DNA, 21 bp, synthetic construct
  misc_feature 1..21: note = oligonucleotide
  source 1..21: mol_type = other DNA
ggagacacccccaagggcctc

SEQ ID NO: 1267 - DNA, 21 bp, synthetic construct
  misc_feature 1..21: note = oligonucleotide
  source 1..21: mol_type = other DNA
gacttcagaggcggccaaaaa

SEQ ID NO: 1268 - DNA, 21 bp, synthetic construct
  misc_feature 1..21: note = oligonucleotide
  source 1..21: mol_type = other DNA
aatttggttgagatctggagg

SEQ ID NO: 1269 - DNA, 21 bp, synthetic construct
  misc_feature 1..21: note = oligonucleotide
  source 1..21: mol_type = other DNA
aaggccaagatgagagaacaa

SEQ ID NO: 1270 - DNA, 21 bp, synthetic construct
  misc_feature 1..21: note = oligonucleotide
  source 1..21: mol_type = other DNA
atgggaaagacctatgcagaa

SEQ ID NO: 1271 - DNA, 21 bp, synthetic construct
  misc_feature 1..21: note = oligonucleotide
  source 1..21: mol_type = other DNA
aaatcagaggcagtgtttttt

SEQ ID NO: 1272 - DNA, 21 bp, synthetic construct
  misc_feature 1..21: note = oligonucleotide
  source 1..21: mol_type = other DNA
ggatgctccaggagacgctaa

SEQ ID NO: 1273 - DNA, 21 bp, synthetic construct
  misc_feature 1..21: note = oligonucleotide
  source 1..21: mol_type = other DNA
aattttaactttctcaccttc

SEQ ID NO: 1274 - DNA, 21 bp, synthetic construct
  misc_feature 1..21: note = oligonucleotide
  source 1..21: mol_type = other DNA
cccaaagacccctgcccggaa

SEQ ID NO: 1275 - DNA, 21 bp, synthetic construct
  misc_feature 1..21: note = oligonucleotide
  source 1..21: mol_type = other DNA
gacattaaaaaaaaaagctta

SEQ ID NO: 1276 - DNA, 21 bp, synthetic construct
  misc_feature 1..21: note = oligonucleotide
  source 1..21: mol_type = other DNA
ccttaggcccaagcccgggct

SEQ ID NO: 1277 - DNA, 21 bp, synthetic construct
  misc_feature 1..21: note = oligonucleotide
  source 1..21: mol_type = other DNA
ggacttcagaggcggccaaaa

SEQ ID NO: 1278 - DNA, 21 bp, synthetic construct
  misc_feature 1..21: note = oligonucleotide
  source 1..21: mol_type = other DNA
tggttagcatttttttttttt

SEQ ID NO: 1279 - DNA, 21 bp, synthetic construct
  misc_feature 1..21: note = oligonucleotide
  source 1..21: mol_type = other DNA
agccacatctgctaggcgagg

SEQ ID NO: 1280 - DNA, 21 bp, synthetic construct
  misc_feature 1..21: note = oligonucleotide
  source 1..21: mol_type = other DNA
ttgcaatcggctaaaaaaaga

SEQ ID NO: 1281 - DNA, 21 bp, synthetic construct
  misc_feature 1..21: note = oligonucleotide
  source 1..21: mol_type = other DNA
tgtgtagaaaaaaagacagcc

SEQ ID NO: 1282 - DNA, 21 bp, synthetic construct
  misc_feature 1..21: note = oligonucleotide
  source 1..21: mol_type = other DNA
gccttgagggctggcgtgccg

SEQ ID NO: 1283 - DNA, 21 bp, synthetic construct
  misc_feature 1..21: note = oligonucleotide
  source 1..21: mol_type = other DNA
aatgttttttaagtacttctc

SEQ ID NO: 1284 - DNA, 21 bp, synthetic construct
  misc_feature 1..21: note = oligonucleotide
  source 1..21: mol_type = other DNA
tttttttcttttaaactgaag

SEQ ID NO: 1285 - DNA, 21 bp, synthetic construct
  misc_feature 1..21: note = oligonucleotide
  source 1..21: mol_type = other DNA
ttcggccttcgaggtcacgtt

SEQ ID NO: 1286 - DNA, 21 bp, synthetic construct
  misc_feature 1..21: note = oligonucleotide
  source 1..21: mol_type = other DNA
tccccccaatgactcccgggc

SEQ ID NO: 1287 - DNA, 21 bp, synthetic construct
  misc_feature 1..21: note = oligonucleotide
  source 1..21: mol_type = other DNA
tcaggctccttttttgatgct

SEQ ID NO: 1288 - DNA, 21 bp, synthetic construct
  misc_feature 1..21: note = oligonucleotide
  source 1..21: mol_type = other DNA
gattttttttttcagattact

SEQ ID NO: 1289 - DNA, 21 bp, synthetic construct
  misc_feature 1..21: note = oligonucleotide
  source 1..21: mol_type = other DNA
gggcacaggggggcagtgctg

SEQ ID NO: 1290 - DNA, 21 bp, synthetic construct
  misc_feature 1..21: note = oligonucleotide
  source 1..21: mol_type = other DNA
ttttttgacaattaaattggg

SEQ ID NO: 1291 - DNA, 21 bp, synthetic construct
  misc_feature 1..21: note = oligonucleotide
  source 1..21: mol_type = other DNA
ggaaacaaaaaaatccttgga

SEQ ID NO: 1292 - DNA, 21 bp, synthetic construct
  misc_feature 1..21: note = oligonucleotide
  source 1..21: mol_type = other DNA
gagccacatctgctaggcgag

SEQ ID NO: 1293 - DNA, 21 bp, synthetic construct
  misc_feature 1..21: note = oligonucleotide
  source 1..21: mol_type = other DNA
gtgctgtgctctctgtatgaa

SEQ ID NO: 1294 - DNA, 21 bp, synthetic construct
  misc_feature 1..21: note = oligonucleotide
  source 1..21: mol_type = other DNA
attaaggaaaaaaagcagtgg

SEQ ID NO: 1295 - DNA, 21 bp, synthetic construct
  misc_feature 1..21: note = oligonucleotide
  source 1..21: mol_type = other DNA
gtttcctgcagtgtttccagg

SEQ ID NO: 1296 - DNA, 21 bp, synthetic construct
  misc_feature 1..21: note = oligonucleotide
  source 1..21: mol_type = other DNA
aaatgcagggtgaacgcggtg

SEQ ID NO: 1297 - DNA, 21 bp, synthetic construct
  misc_feature 1..21: note = oligonucleotide
  source 1..21: mol_type = other DNA
ggctgcagactagagtgggga

SEQ ID NO: 1298 - DNA, 21 bp, synthetic construct
  misc_feature 1..21: note = oligonucleotide
  source 1..21: mol_type = other DNA
ccccagtgccagtcggggccc

SEQ ID NO: 1299 - DNA, 21 bp, synthetic construct
  misc_feature 1..21: note = oligonucleotide
  source 1..21: mol_type = other DNA
gagggcttggggcgtactccg

SEQ ID NO: 1300 - DNA, 21 bp, synthetic construct
  misc_feature 1..21: note = oligonucleotide
  source 1..21: mol_type = other DNA
cattcttgctttaaaaaaaaa

SEQ ID NO: 1301 - DNA, 21 bp, synthetic construct
  misc_feature 1..21: note = oligonucleotide
  source 1..21: mol_type = other DNA
gactagagtggggagagagga

SEQ ID NO: 1302 - DNA, 21 bp, synthetic construct
  misc_feature 1..21: note = oligonucleotide
  source 1..21: mol_type = other DNA
gagcaagcgccaaaaaaggag

SEQ ID NO: 1303 - DNA, 21 bp, synthetic construct
  misc_feature 1..21: note = oligonucleotide
  source 1..21: mol_type = other DNA
gatacctataatatagaaatc

SEQ ID NO: 1304 - DNA, 21 bp, synthetic construct
  misc_feature 1..21: note = oligonucleotide
  source 1..21: mol_type = other DNA
atatagttttgctttttttgg

SEQ ID NO: 1305 - DNA, 21 bp, synthetic construct
  misc_feature 1..21: note = oligonucleotide
  source 1..21: mol_type = other DNA
tttgttttgttttggtaatga

SEQ ID NO: 1306 - DNA, 21 bp, synthetic construct
  misc_feature 1..21: note = oligonucleotide
  source 1..21: mol_type = other DNA
gcggccagccaggaccccccc

SEQ ID NO: 1307 - DNA, 21 bp, synthetic construct
  misc_feature 1..21: note = oligonucleotide
  source 1..21: mol_type = other DNA
cccccccccaataatttcgcc

SEQ ID NO: 1308 - DNA, 21 bp, synthetic construct
  misc_feature 1..21: note = oligonucleotide
  source 1..21: mol_type = other DNA
cctccggtcccggcacccact

SEQ ID NO: 1309 - DNA, 21 bp, synthetic construct
  misc_feature 1..21: note = oligonucleotide
  source 1..21: mol_type = other DNA
taagcttttttttttaatgtc

SEQ ID NO: 1310 - DNA, 21 bp, synthetic construct
  misc_feature 1..21: note = oligonucleotide
  source 1..21: mol_type = other DNA
cccgtgtgagtcggtgaacga

SEQ ID NO: 1311 - DNA, 21 bp, synthetic construct
  misc_feature 1..21: note = oligonucleotide
  source 1..21: mol_type = other DNA
gcatcccccgccgcagcagag

SEQ ID NO: 1312 - DNA, 21 bp, synthetic construct
  misc_feature 1..21: note = oligonucleotide
  source 1..21: mol_type = other DNA
ccaacatggtggctcctagtt

SEQ ID NO: 1313 - DNA, 21 bp, synthetic construct
  misc_feature 1..21: note = oligonucleotide
  source 1..21: mol_type = other DNA
aggggggatggtggtgcactc

SEQ ID NO: 1314 - DNA, 21 bp, synthetic construct
  misc_feature 1..21: note = oligonucleotide
  source 1..21: mol_type = other DNA
aaaaaagccgtctggagagca

SEQ ID NO: 1315 - DNA, 21 bp, synthetic construct
  misc_feature 1..21: note = oligonucleotide
  source 1..21: mol_type = other DNA
gctcaacagcaaaggtgtctg

SEQ ID NO: 1316 - DNA, 21 bp, synthetic construct
  misc_feature 1..21: note = oligonucleotide
  source 1..21: mol_type = other DNA
ttgcaatcggctaaaaaaaga

SEQ ID NO: 1317 - DNA, 21 bp, synthetic construct
  misc_feature 1..21: note = oligonucleotide
  source 1..21: mol_type = other DNA
gagctgacaaaaaaaaaagtg

SEQ ID NO: 1318 - DNA, 21 bp, synthetic construct
  misc_feature 1..21: note = oligonucleotide
  source 1..21: mol_type = other DNA
ttttttcttttaaactgaagg

SEQ ID NO: 1319 - DNA, 21 bp, synthetic construct
  misc_feature 1..21: note = oligonucleotide
  source 1..21: mol_type = other DNA
ccagattttttttttcaaact

SEQ ID NO: 1320 - DNA, 21 bp, synthetic construct
  misc_feature 1..21: note = oligonucleotide
  source 1..21: mol_type = other DNA
tctcttgcaatcggctaaaaa

SEQ ID NO: 1321 - DNA, 21 bp, synthetic construct
  misc_feature 1..21: note = oligonucleotide
  source 1..21: mol_type = other DNA
ttgatgtttttaaactttttt

SEQ ID NO: 1322 - DNA, 21 bp, synthetic construct
  misc_feature 1..21: note = oligonucleotide
  source 1..21: mol_type = other DNA
ttatggcttaaaaagaaaaaa

SEQ ID NO: 1323 - DNA, 21 bp, synthetic construct
  misc_feature 1..21: note = oligonucleotide
  source 1..21: mol_type = other DNA
cataaaactgattttttttgc

SEQ ID NO: 1324 - DNA, 21 bp, synthetic construct
  misc_feature 1..21: note = oligonucleotide
  source 1..21: mol_type = other DNA
taaaaaaaaaaagaagaaaag

SEQ ID NO: 1325 - DNA, 21 bp, synthetic construct
  misc_feature 1..21: note = oligonucleotide
  source 1..21: mol_type = other DNA
tgaggaattgatagcgtatct

SEQ ID NO: 1326 - DNA, 21 bp, synthetic construct
  misc_feature 1..21: note = oligonucleotide
  source 1..21: mol_type = other DNA
agcggccagccaggacccccc

SEQ ID NO: 1327 - DNA, 21 bp, synthetic construct
  misc_feature 1..21: note = oligonucleotide
  source 1..21: mol_type = other DNA
aaatcccttcacattttaact

SEQ ID NO: 1328 - DNA, 21 bp, synthetic construct
  misc_feature 1..21: note = oligonucleotide
  source 1..21: mol_type = other DNA
cttcatattccgacataaaaa

SEQ ID NO: 1329 - DNA, 21 bp, synthetic construct
  misc_feature 1..21: note = oligonucleotide
  source 1..21: mol_type = other DNA
aaaaatgcagggtgaacgcgg

SEQ ID NO: 1330 - DNA, 21 bp, synthetic construct
  misc_feature 1..21: note = oligonucleotide
  source 1..21: mol_type = other DNA
aaaaaagacgtttttaaaacg

SEQ ID NO: 1331 - DNA, 21 bp, synthetic construct
  misc_feature 1..21: note = oligonucleotide
  source 1..21: mol_type = other DNA
gtggtctgtctatcaagaagg

SEQ ID NO: 1332 - DNA, 21 bp, synthetic construct
  misc_feature 1..21: note = oligonucleotide
  source 1..21: mol_type = other DNA
tcccgtgtacaaaaaaggtgg

SEQ ID NO: 1333 - DNA, 21 bp, synthetic construct
  misc_feature 1..21: note = oligonucleotide
  source 1..21: mol_type = other DNA
gttttgatttttttttttaaa

SEQ ID NO: 1334 - DNA, 21 bp, synthetic construct
  misc_feature 1..21: note = oligonucleotide
  source 1..21: mol_type = other DNA
tgattctacttcaggaggcac

SEQ ID NO: 1335 - DNA, 21 bp, synthetic construct
  misc_feature 1..21: note = oligonucleotide
  source 1..21: mol_type = other DNA
taggagccacatctgctaggc

SEQ ID NO: 1336 - DNA, 21 bp, synthetic construct
  misc_feature 1..21: note = oligonucleotide
  source 1..21: mol_type = other DNA
ttatgatgaaatgcagaaaaa

SEQ ID NO: 1337 - DNA, 21 bp, synthetic construct
  misc_feature 1..21: note = oligonucleotide
  source 1..21: mol_type = other DNA
accaaaacaaatttttttccc

SEQ ID NO: 1338 - DNA, 21 bp, synthetic construct
  misc_feature 1..21: note = oligonucleotide
  source 1..21: mol_type = other DNA
ggggactgccatctggggcac

SEQ ID NO: 1339 - DNA, 21 bp, synthetic construct
  misc_feature 1..21: note = oligonucleotide
  source 1..21: mol_type = other DNA
ttttttttcttgttgcttaag

SEQ ID NO: 1340 - DNA, 21 bp, synthetic construct
  misc_feature 1..21: note = oligonucleotide
  source 1..21: mol_type = other DNA
tctcttttttttcctagtctt

SEQ ID NO: 1341 - DNA, 21 bp, synthetic construct
  misc_feature 1..21: note = oligonucleotide
  source 1..21: mol_type = other DNA
aaaaaaatgacattagattta

SEQ ID NO: 1342 - DNA, 21 bp, synthetic construct
  misc_feature 1..21: note = oligonucleotide
  source 1..21: mol_type = other DNA
cgggcaggggtctttgggggg

SEQ ID NO: 1343 - DNA, 21 bp, synthetic construct
  misc_feature 1..21: note = oligonucleotide
  source 1..21: mol_type = other DNA
tgccttttttttattgctctg

SEQ ID NO: 1344 - DNA, 21 bp, synthetic construct
  misc_feature 1..21: note = oligonucleotide
  source 1..21: mol_type = other DNA
ggcaccccctgcaccacaagc

SEQ ID NO: 1345 - DNA, 21 bp, synthetic construct
  misc_feature 1..21: note = oligonucleotide
  source 1..21: mol_type = other DNA
attaattgggtaaaaaaaaaa

SEQ ID NO: 1346 - DNA, 21 bp, synthetic construct
  misc_feature 1..21: note = oligonucleotide
  source 1..21: mol_type = other DNA
ccaggggggatggtggtgcac

SEQ ID NO: 1347 - DNA, 21 bp, synthetic construct
  misc_feature 1..21: note = oligonucleotide
  source 1..21: mol_type = other DNA
ctgctgcggcgggggatgctc

SEQ ID NO: 1348 - DNA, 21 bp, synthetic construct
  misc_feature 1..21: note = oligonucleotide
  source 1..21: mol_type = other DNA
ggggggatggtggtgcactcg

SEQ ID NO: 1349 - DNA, 21 bp, synthetic construct
  misc_feature 1..21: note = oligonucleotide
  source 1..21: mol_type = other DNA
ctgctaggcgaggaggaggaa

SEQ ID NO: 1350 - DNA, 21 bp, synthetic construct
  misc_feature 1..21: note = oligonucleotide
  source 1..21: mol_type = other DNA
aggagccacatctgctaggcg

SEQ ID NO: 1351 - DNA, 21 bp, synthetic construct
  misc_feature 1..21: note = oligonucleotide
  source 1..21: mol_type = other DNA
aaaaaaagaattttcattttg

SEQ ID NO: 1352 - DNA, 21 bp, synthetic construct
  misc_feature 1..21: note = oligonucleotide
  source 1..21: mol_type = other DNA
aaaagaaaaaaaaggctgttt

SEQ ID NO: 1353 - DNA, 21 bp, synthetic construct
  misc_feature 1..21: note = oligonucleotide
  source 1..21: mol_type = other DNA
tggagctgacaaaaaaaaaag

SEQ ID NO: 1354 - DNA, 21 bp, synthetic construct
  misc_feature 1..21: note = oligonucleotide
  source 1..21: mol_type = other DNA
cggtgagaccacggatagctg

SEQ ID NO: 1355 - DNA, 21 bp, synthetic construct
  misc_feature 1..21: note = oligonucleotide
  source 1..21: mol_type = other DNA
accggggatggaagtccaggc

SEQ ID NO: 1356 - DNA, 21 bp, synthetic construct
  misc_feature 1..21: note = oligonucleotide
  source 1..21: mol_type = other DNA
accgactatagtcaaaaaaaa

SEQ ID NO: 1357 - DNA, 21 bp, synthetic construct
  misc_feature 1..21: note = oligonucleotide
  source 1..21: mol_type = other DNA
ttttcagtacttctaagatca

SEQ ID NO: 1358 - DNA, 21 bp, synthetic construct
  misc_feature 1..21: note = oligonucleotide
  source 1..21: mol_type = other DNA
aagtgggtgagaagctaagtg

SEQ ID NO: 1359 - DNA, 21 bp, synthetic construct
  misc_feature 1..21: note = oligonucleotide
  source 1..21: mol_type = other DNA
aaaaaaaatgacattagattt

SEQ ID NO: 1360 - DNA, 21 bp, synthetic construct
  misc_feature 1..21: note = oligonucleotide
  source 1..21: mol_type = other DNA
ccaccttaacattattagaca

SEQ ID NO: 1361 - DNA, 21 bp, synthetic construct
  misc_feature 1..21: note = oligonucleotide
  source 1..21: mol_type = other DNA
agaggtaaaaaacgacggcgg

SEQ ID NO: 1362 - DNA, 21 bp, synthetic construct
  misc_feature 1..21: note = oligonucleotide
  source 1..21: mol_type = other DNA
tggctcaacagcaaaggtgtc

SEQ ID NO: 1363 - DNA, 21 bp, synthetic construct
  misc_feature 1..21: note = oligonucleotide
  source 1..21: mol_type = other DNA
aaatgaataatgtgtatgagg

SEQ ID NO: 1364 - DNA, 21 bp, synthetic construct
  misc_feature 1..21: note = oligonucleotide
  source 1..21: mol_type = other DNA
aatggattttttttaagcaag

SEQ ID NO: 1365 - DNA, 21 bp, synthetic construct
  misc_feature 1..21: note = oligonucleotide
  source 1..21: mol_type = other DNA
atcttcattaaagaatttaat

SEQ ID NO: 1366 - DNA, 21 bp, synthetic construct
  misc_feature 1..21: note = oligonucleotide
  source 1..21: mol_type = other DNA
tttcacttttttttcctatta

SEQ ID NO: 1367 - DNA, 21 bp, synthetic construct
  misc_feature 1..21: note = oligonucleotide
  source 1..21: mol_type = other DNA
agcggaaaaaaaaaaagaggt

SEQ ID NO: 1368 - DNA, 21 bp, synthetic construct
  misc_feature 1..21: note = oligonucleotide
  source 1..21: mol_type = other DNA
tcttcattaaagaatttaata

SEQ ID NO: 1369 - DNA, 21 bp, synthetic construct
  misc_feature 1..21: note = oligonucleotide
  source 1..21: mol_type = other DNA
ggtaggagccacatctgctag

SEQ ID NO: 1370 - DNA, 21 bp, synthetic construct
  misc_feature 1..21: note = oligonucleotide
  source 1..21: mol_type = other DNA
gccttgagggctggcgtgccg

SEQ ID NO: 1371 - DNA, 21 bp, synthetic construct
  misc_feature 1..21: note = oligonucleotide
  source 1..21: mol_type = other DNA
ccgactatagtcaaaaaaaaa

SEQ ID NO: 1372 - DNA, 21 bp, synthetic construct
  misc_feature 1..21: note = oligonucleotide
  source 1..21: mol_type = other DNA
gacaggattttttttttccca

SEQ ID NO: 1373 - DNA, 21 bp, synthetic construct
  misc_feature 1..21: note = oligonucleotide
  source 1..21: mol_type = other DNA
cacaagcagggggcactgtac

SEQ ID NO: 1374 - DNA, 21 bp, synthetic construct
  misc_feature 1..21: note = oligonucleotide
  source 1..21: mol_type = other DNA
tggcaccccctgcaccacaag

SEQ ID NO: 1375 - DNA, 21 bp, synthetic construct
  misc_feature 1..21: note = oligonucleotide
  source 1..21: mol_type = other DNA
tttggtggtctgtctatcaag

SEQ ID NO: 1376 - DNA, 21 bp, synthetic construct
  misc_feature 1..21: note = oligonucleotide
  source 1..21: mol_type = other DNA
aagaccaccagccctgagcct

SEQ ID NO: 1377 - DNA, 21 bp, synthetic construct
  misc_feature 1..21: note = oligonucleotide
  source 1..21: mol_type = other DNA
tgctaggcgaggaggaggaag

SEQ ID NO: 1378 - DNA, 21 bp, synthetic construct
  misc_feature 1..21: note = oligonucleotide
  source 1..21: mol_type = other DNA
tctgctaggcgaggaggagga

SEQ ID NO: 1379 - DNA, 21 bp, synthetic construct
  misc_feature 1..21: note = oligonucleotide
  source 1..21: mol_type = other DNA
ttttttgtcttaaaattttta

SEQ ID NO: 1380 - DNA, 21 bp, synthetic construct
  misc_feature 1..21: note = oligonucleotide
  source 1..21: mol_type = other DNA
tatgatgaaatgcagaaaaaa

SEQ ID NO: 1381 - DNA, 21 bp, synthetic construct
  misc_feature 1..21: note = oligonucleotide
  source 1..21: mol_type = other DNA
tgttttaaacccatttttttt

SEQ ID NO: 1382 - DNA, 21 bp, synthetic construct
  misc_feature 1..21: note = oligonucleotide
  source 1..21: mol_type = other DNA
atggagctgacaaaaaaaaaa

SEQ ID NO: 1383 - DNA, 21 bp, synthetic construct
  misc_feature 1..21: note = oligonucleotide
  source 1..21: mol_type = other DNA
acggtccccccacaccaagaa

SEQ ID NO: 1384 - DNA, 21 bp, synthetic construct
  misc_feature 1..21: note = oligonucleotide
  source 1..21: mol_type = other DNA
cccagggatggggcgtcaggg

SEQ ID NO: 1385 - DNA, 21 bp, synthetic construct
  misc_feature 1..21: note = oligonucleotide
  source 1..21: mol_type = other DNA
ttttttagcaccttattttcc

SEQ ID NO: 1386 - DNA, 21 bp, synthetic construct
  misc_feature 1..21: note = oligonucleotide
  source 1..21: mol_type = other DNA
tcaggactcacagctcttcaa

SEQ ID NO: 1387 - DNA, 21 bp, synthetic construct
  misc_feature 1..21: note = oligonucleotide
  source 1..21: mol_type = other DNA
gagaacttcacaaaagacacg

SEQ ID NO: 1388 - DNA, 21 bp, synthetic construct
  misc_feature 1..21: note = oligonucleotide
  source 1..21: mol_type = other DNA
gggagcccaggggtggctaca

SEQ ID NO: 1389 - DNA, 21 bp, synthetic construct
  misc_feature 1..21: note = oligonucleotide
  source 1..21: mol_type = other DNA
cttacatggtggtgagcagag

SEQ ID NO: 1390 - DNA, 21 bp, synthetic construct
  misc_feature 1..21: note = oligonucleotide
  source 1..21: mol_type = other DNA
ggtgagcagaggggggtgtag

SEQ ID NO: 1391 - DNA, 21 bp, synthetic construct
  misc_feature 1..21: note = oligonucleotide
  source 1..21: mol_type = other DNA
cccataaagactgcatttttt

SEQ ID NO: 1392 - DNA, 21 bp, synthetic construct
  misc_feature 1..21: note = oligonucleotide
  source 1..21: mol_type = other DNA
aaaaattccttagggatatct

SEQ ID NO: 1393 - DNA, 21 bp, synthetic construct
  misc_feature 1..21: note = oligonucleotide
  source 1..21: mol_type = other DNA
ttaatttaaaaaaaaaaagaa

SEQ ID NO: 1394 - DNA, 21 bp, synthetic construct
  misc_feature 1..21: note = oligonucleotide
  source 1..21: mol_type = other DNA
agaccaccagccctgagcctc

SEQ ID NO: 1395 - DNA, 21 bp, synthetic construct
  misc_feature 1..21: note = oligonucleotide
  source 1..21: mol_type = other DNA
aagcagacaaatagactttct

SEQ ID NO: 1396 - DNA, 21 bp, synthetic construct
  misc_feature 1..21: note = oligonucleotide
  source 1..21: mol_type = other DNA
atctgctaggcgaggaggagg

SEQ ID NO: 1397 - DNA, 21 bp, synthetic construct
  misc_feature 1..21: note = oligonucleotide
  source 1..21: mol_type = other DNA
gggattttttttttaaacgat

SEQ ID NO: 1398 - DNA, 21 bp, synthetic construct
  misc_feature 1..21: note = oligonucleotide
  source 1..21: mol_type = other DNA
cgagtggttaaaaaaagaccg

SEQ ID NO: 1399 - DNA, 21 bp, synthetic construct
  misc_feature 1..21: note = oligonucleotide
  source 1..21: mol_type = other DNA
cggtgagaccacggatagctg

SEQ ID NO: 1400 - DNA, 21 bp, synthetic construct
  misc_feature 1..21: note = oligonucleotide
  source 1..21: mol_type = other DNA
tgtgtgtgtgtattttcactg

SEQ ID NO: 1401 - DNA, 21 bp, synthetic construct
  misc_feature 1..21: note = oligonucleotide
  source 1..21: mol_type = other DNA
ttctttttttttttaagggcc

SEQ ID NO: 1402 - DNA, 21 bp, synthetic construct
  misc_feature 1..21: note = oligonucleotide
  source 1..21: mol_type = other DNA
acttccatccccggtcatctt

SEQ ID NO: 1403 - DNA, 21 bp, synthetic construct
  misc_feature 1..21: note = oligonucleotide
  source 1..21: mol_type = other DNA
cggtccccccacaccaagaac

SEQ ID NO: 1404 - DNA, 21 bp, synthetic construct
  misc_feature 1..21: note = oligonucleotide
  source 1..21: mol_type = other DNA
ggacactttctccaggaggga

SEQ ID NO: 1405 - DNA, 21 bp, synthetic construct
  misc_feature 1..21: note = oligonucleotide
  source 1..21: mol_type = other DNA
tggtgttttttgagatattct

SEQ ID NO: 1406 - DNA, 21 bp, synthetic construct
  misc_feature 1..21: note = oligonucleotide
  source 1..21: mol_type = other DNA
tctcttttttttcctagtctt

SEQ ID NO: 1407 - DNA, 21 bp, synthetic construct
  misc_feature 1..21: note = oligonucleotide
  source 1..21: mol_type = other DNA
ggtacagggaactggactgtc

SEQ ID NO: 1408 - DNA, 21 bp, synthetic construct
  misc_feature 1..21: note = oligonucleotide
  source 1..21: mol_type = other DNA
ttgcatatgcattttttttta

SEQ ID NO: 1409 - DNA, 21 bp, synthetic construct
  misc_feature 1..21: note = oligonucleotide
  source 1..21: mol_type = other DNA
agaagatgggtattccttttt

SEQ ID NO: 1410 - DNA, 21 bp, synthetic construct
  misc_feature 1..21: note = oligonucleotide
  source 1..21: mol_type = other DNA
ctctgaaggaagtgatgttag

SEQ ID NO: 1411 - DNA, 21 bp, synthetic construct
  misc_feature 1..21: note = oligonucleotide
  source 1..21: mol_type = other DNA
attaaaaaaaaatcttcatta

SEQ ID NO: 1412 - DNA, 21 bp, synthetic construct
  misc_feature 1..21: note = oligonucleotide
  source 1..21: mol_type = other DNA
ccccatccctggggttggtgg

SEQ ID NO: 1413 - DNA, 21 bp, synthetic construct
  misc_feature 1..21: note = oligonucleotide
  source 1..21: mol_type = other DNA
cgtcaggactcacagctcttc

SEQ ID NO: 1414 - DNA, 21 bp, synthetic construct
  misc_feature 1..21: note = oligonucleotide
  source 1..21: mol_type = other DNA
gtgtgtgtgtgtattttcact

SEQ ID NO: 1415 - DNA, 21 bp, synthetic construct
  misc_feature 1..21: note = oligonucleotide
  source 1..21: mol_type = other DNA
cttattttctagaaaacgtta

SEQ ID NO: 1416 - DNA, 21 bp, synthetic construct
  misc_feature 1..21: note = oligonucleotide
  source 1..21: mol_type = other DNA
gggggttgcactggagcgtca

SEQ ID NO: 1417 - DNA, 21 bp, synthetic construct
  misc_feature 1..21: note = oligonucleotide
  source 1..21: mol_type = other DNA
aaaaaatgaataatgtgtatg

SEQ ID NO: 1418 - DNA, 21 bp, synthetic construct
  misc_feature 1..21: note = oligonucleotide
  source 1..21: mol_type = other DNA
ttttttttctctttttaatac

SEQ ID NO: 1419 - DNA, 21 bp, synthetic construct
  misc_feature 1..21: note = oligonucleotide
  source 1..21: mol_type = other DNA
aaaaagtgggtgagaagctaa

SEQ ID NO: 1420 - DNA, 21 bp, synthetic construct
  misc_feature 1..21: note = oligonucleotide
  source 1..21: mol_type = other DNA
gggattggcagggggtggcat

SEQ ID NO: 1421 - DNA, 21 bp, synthetic construct
  misc_feature 1..21: note = oligonucleotide
  source 1..21: mol_type = other DNA
aaaaaaaataaagtgtgtgcc

SEQ ID NO: 1422 - DNA, 21 bp, synthetic construct
  misc_feature 1..21: note = oligonucleotide
  source 1..21: mol_type = other DNA
gtcaggactcacagctcttca

SEQ ID NO: 1423 - DNA, 21 bp, synthetic construct
  misc_feature 1..21: note = oligonucleotide
  source 1..21: mol_type = other DNA
ctgcacccccccaccaacccc

SEQ ID NO: 1424 - DNA, 21 bp, synthetic construct
  misc_feature 1..21: note = oligonucleotide
  source 1..21: mol_type = other DNA
gaggggacatgcaggcaggca

SEQ ID NO: 1425 - DNA, 21 bp, synthetic construct
  misc_feature 1..21: note = oligonucleotide
  source 1..21: mol_type = other DNA
ggcggctgaagggggtgtggc

SEQ ID NO: 1426 - DNA, 21 bp, synthetic construct
  misc_feature 1..21: note = oligonucleotide
  source 1..21: mol_type = other DNA
aaatgcagggtgaacgcggtg

SEQ ID NO: 1427 - DNA, 21 bp, synthetic construct
  misc_feature 1..21: note = oligonucleotide
  source 1..21: mol_type = other DNA
caggactcacagctcttcaag

SEQ ID NO: 1428 - DNA, 21 bp, synthetic construct
  misc_feature 1..21: note = oligonucleotide
  source 1..21: mol_type = other DNA
gtctttagcaaagtttagact

SEQ ID NO: 1429 - DNA, 21 bp, synthetic construct
  misc_feature 1..21: note = oligonucleotide
  source 1..21: mol_type = other DNA
atctgggttggggggaagggg

SEQ ID NO: 1430 - DNA, 21 bp, synthetic construct
  misc_feature 1..21: note = oligonucleotide
  source 1..21: mol_type = other DNA
caaaaaagcagctgctttcag

SEQ ID NO: 1431 - DNA, 21 bp, synthetic construct
  misc_feature 1..21: note = oligonucleotide
  source 1..21: mol_type = other DNA
tggtgcactcggggaggcggg

SEQ ID NO: 1432 - DNA, 21 bp, synthetic construct
  misc_feature 1..21: note = oligonucleotide
  source 1..21: mol_type = other DNA
accggcggcctggacccccca

SEQ ID NO: 1433 - DNA, 21 bp, synthetic construct
  misc_feature 1..21: note = oligonucleotide
  source 1..21: mol_type = other DNA
cccccttcctccctggtgagg

SEQ ID NO: 1434 - DNA, 21 bp, synthetic construct
  misc_feature 1..21: note = oligonucleotide
  source 1..21: mol_type = other DNA
gactgtttgaaaaaaaaaaat

SEQ ID NO: 1435 - DNA, 21 bp, synthetic construct
  misc_feature 1..21: note = oligonucleotide
  source 1..21: mol_type = other DNA
gggggcccccatgactctggg

SEQ ID NO: 1436 - DNA, 21 bp, synthetic construct
  misc_feature 1..21: note = oligonucleotide
  source 1..21: mol_type = other DNA
attcttgagagagcatagtgt

SEQ ID NO: 1437 - DNA, 21 bp, synthetic construct
  misc_feature 1..21: note = oligonucleotide
  source 1..21: mol_type = other DNA
tgatggtttttttttacatta

SEQ ID NO: 1438 - DNA, 21 bp, synthetic construct
  misc_feature 1..21: note = oligonucleotide
  source 1..21: mol_type = other DNA
actaccgtcggcgcaaaaaaa

SEQ ID NO: 1439 - DNA, 21 bp, synthetic construct
  misc_feature 1..21: note = oligonucleotide
  source 1..21: mol_type = other DNA
aaaacacgttttcgaaaaaaa

SEQ ID NO: 1440 - DNA, 21 bp, synthetic construct
  misc_feature 1..21: note = oligonucleotide
  source 1..21: mol_type = other DNA
cataaaactgattttttttgc

SEQ ID NO: 1441 - DNA, 21 bp, synthetic construct
  misc_feature 1..21: note = oligonucleotide
  source 1..21: mol_type = other DNA
cgctgcaactacagcatccgg

SEQ ID NO: 1442 - DNA, 21 bp, synthetic construct
  misc_feature 1..21: note = oligonucleotide
  source 1..21: mol_type = other DNA
ggtgccagcctgccatgcgtg

SEQ ID NO: 1443 - DNA, 21 bp, synthetic construct
  misc_feature 1..21: note = oligonucleotide
  source 1..21: mol_type = other DNA
ttaaggggggtgacatttctt

SEQ ID NO: 1444 - DNA, 21 bp, synthetic construct
  misc_feature 1..21: note = oligonucleotide
  source 1..21: mol_type = other DNA
ctggaccccccatgggagacg

SEQ ID NO: 1445 - DNA, 21 bp, synthetic construct
  misc_feature 1..21: note = oligonucleotide
  source 1..21: mol_type = other DNA
agtcaaaaaacgaaagagaag

SEQ ID NO: 1446 - DNA, 21 bp, synthetic construct
  misc_feature 1..21: note = oligonucleotide
  source 1..21: mol_type = other DNA
gtgccagcctgccatgcgtgc

SEQ ID NO: 1447 - DNA, 21 bp, synthetic construct
  misc_feature 1..21: note = oligonucleotide
  source 1..21: mol_type = other DNA
ctttagcaaaggttggactga

SEQ ID NO: 1448 - DNA, 21 bp, synthetic construct
  misc_feature 1..21: note = oligonucleotide
  source 1..21: mol_type = other DNA
aaaaaacgacggcggcggaag

SEQ ID NO: 1449 - DNA, 21 bp, synthetic construct
  misc_feature 1..21: note = oligonucleotide
  source 1..21: mol_type = other DNA
tgccatcggagggggggtggc

SEQ ID NO: 1450 - DNA, 21 bp, synthetic construct
  misc_feature 1..21: note = oligonucleotide
  source 1..21: mol_type = other DNA
ttcttgagagagcatagtgtg

SEQ ID NO: 1451 - DNA, 21 bp, synthetic construct
  misc_feature 1..21: note = oligonucleotide
  source 1..21: mol_type = other DNA
gctccttcctcctggcttccc

SEQ ID NO: 1452 - DNA, 21 bp, synthetic construct
  misc_feature 1..21: note = oligonucleotide
  source 1..21: mol_type = other DNA
gactaccgtcggcgcaaaaaa

SEQ ID NO: 1453 - DNA, 21 bp, synthetic construct
  misc_feature 1..21: note = oligonucleotide
  source 1..21: mol_type = other DNA
taggcaaaacacgagaagagc

SEQ ID NO: 1454 - DNA, 21 bp, synthetic construct
  misc_feature 1..21: note = oligonucleotide
  source 1..21: mol_type = other DNA
aaaatgcatatgcaatgtccc

SEQ ID NO: 1455 - DNA, 21 bp, synthetic construct
  misc_feature 1..21: note = oligonucleotide
  source 1..21: mol_type = other DNA
actggttagcatttttttttt

SEQ ID NO: 1456 - DNA, 21 bp, synthetic construct
  misc_feature 1..21: note = oligonucleotide
  source 1..21: mol_type = other DNA
cggatctcacgggagttcctc

SEQ ID NO: 1457 - DNA, 21 bp, synthetic construct
  misc_feature 1..21: note = oligonucleotide
  source 1..21: mol_type = other DNA
actcggggaggcgggaagagg

SEQ ID NO: 1458 - DNA, 21 bp, synthetic construct
  misc_feature 1..21: note = oligonucleotide
  source 1..21: mol_type = other DNA
tggtggaaggcgtcaaagtgt

SEQ ID NO: 1459 - DNA, 21 bp, synthetic construct
  misc_feature 1..21: note = oligonucleotide
  source 1..21: mol_type = other DNA
tggggttcggcgaccgtcagg

SEQ ID NO: 1460 - DNA, 21 bp, synthetic construct
  misc_feature 1..21: note = oligonucleotide
  source 1..21: mol_type = other DNA
ggggattggcagggggtggca

SEQ ID NO: 1461 - DNA, 21 bp, synthetic construct
  misc_feature 1..21: note = oligonucleotide
  source 1..21: mol_type = other DNA
gagcaagcgccaaaaaaggag

SEQ ID NO: 1462 - DNA, 21 bp, synthetic construct
  misc_feature 1..21: note = oligonucleotide
  source 1..21: mol_type = other DNA
ggagggctctgatcccccctg

SEQ ID NO: 1463 - DNA, 21 bp, synthetic construct
  misc_feature 1..21: note = oligonucleotide
  source 1..21: mol_type = other DNA
gctcgagtggttaaaaaaaga

SEQ ID NO: 1464 - DNA, 21 bp, synthetic construct
  misc_feature 1..21: note = oligonucleotide
  source 1..21: mol_type = other DNA
gaggtggcggcggggccaccg

SEQ ID NO: 1465 - DNA, 21 bp, synthetic construct
  misc_feature 1..21: note = oligonucleotide
  source 1..21: mol_type = other DNA
ccaaggcgcggtgccccagag

SEQ ID NO: 1466 - DNA, 21 bp, synthetic construct
  misc_feature 1..21: note = oligonucleotide
  source 1..21: mol_type = other DNA
ggtgaggtcggagccagaggg

SEQ ID NO: 1467 - DNA, 21 bp, synthetic construct
  misc_feature 1..21: note = oligonucleotide
  source 1..21: mol_type = other DNA
ggtgagcagaggggggtgtag

SEQ ID NO: 1468 - DNA, 21 bp, synthetic construct
  misc_feature 1..21: note = oligonucleotide
  source 1..21: mol_type = other DNA
ccaaaaaagcagctgctttca

SEQ ID NO: 1469 - DNA, 21 bp, synthetic construct
  misc_feature 1..21: note = oligonucleotide
  source 1..21: mol_type = other DNA
gggccacgatcatatagctct

SEQ ID NO: 1470 - DNA, 21 bp, synthetic construct
  misc_feature 1..21: note = oligonucleotide
  source 1..21: mol_type = other DNA
ttctgagcgcataaagctaag

SEQ ID NO: 1471 - DNA, 21 bp, synthetic construct
  misc_feature 1..21: note = oligonucleotide
  source 1..21: mol_type = other DNA
gggggggactaaaaggagggc

SEQ ID NO: 1472 - DNA, 21 bp, synthetic construct
  misc_feature 1..21: note = oligonucleotide
  source 1..21: mol_type = other DNA
gtttctgagcgcataaagcta

SEQ ID NO: 1473 - DNA, 21 bp, synthetic construct
  misc_feature 1..21: note = oligonucleotide
  source 1..21: mol_type = other DNA
cctcatgcccaccccctggct

SEQ ID NO: 1474 - DNA, 21 bp, synthetic construct
  misc_feature 1..21: note = oligonucleotide
  source 1..21: mol_type = other DNA
tttctttttttttctcttgac

SEQ ID NO: 1475 - DNA, 21 bp, synthetic construct
  misc_feature 1..21: note = oligonucleotide
  source 1..21: mol_type = other DNA
acggccgcctcgttcatttcg

SEQ ID NO: 1476 - DNA, 21 bp, synthetic construct
  misc_feature 1..21: note = oligonucleotide
  source 1..21: mol_type = other DNA
aaaagaaaaaaaaggctgttt

SEQ ID NO: 1477 - DNA, 21 bp, synthetic construct
  misc_feature 1..21: note = oligonucleotide
  source 1..21: mol_type = other DNA
aaacgtattttttttttgagt

SEQ ID NO: 1478 - DNA, 21 bp, synthetic construct
  misc_feature 1..21: note = oligonucleotide
  source 1..21: mol_type = other DNA
ggtggaaggcgtcaaagtgta

SEQ ID NO: 1479 - DNA, 21 bp, synthetic construct
  misc_feature 1..21: note = oligonucleotide
  source 1..21: mol_type = other DNA
gagatgggaaaaaaagtgggt

SEQ ID NO: 1480 - DNA, 21 bp, synthetic construct
  misc_feature 1..21: note = oligonucleotide
  source 1..21: mol_type = other DNA
ctgacaggagacgcactcggc

SEQ ID NO: 1481 - DNA, 21 bp, synthetic construct
  misc_feature 1..21: note = oligonucleotide
  source 1..21: mol_type = other DNA
aaaaaaaaaaagagagagagc

SEQ ID NO: 1482 - DNA, 21 bp, synthetic construct
  misc_feature 1..21: note = oligonucleotide
  source 1..21: mol_type = other DNA
tgttcaaatgattttaattat

SEQ ID NO: 1483 - DNA, 21 bp, synthetic construct
  misc_feature 1..21: note = oligonucleotide
  source 1..21: mol_type = other DNA
tatatataaatacccctacat

SEQ ID NO: 1484 - DNA, 21 bp, synthetic construct
  misc_feature 1..21: note = oligonucleotide
  source 1..21: mol_type = other DNA
acgagacgtgagtggaggcca

SEQ ID NO: 1485 - DNA, 21 bp, synthetic construct
  misc_feature 1..21: note = oligonucleotide
  source 1..21: mol_type = other DNA
gagaccacggagccaccccca

SEQ ID NO: 1486 - DNA, 21 bp, synthetic construct
  misc_feature 1..21: note = oligonucleotide
  source 1..21: mol_type = other DNA
aggggggactgatgcagtgtg

SEQ ID NO: 1487 - DNA, 21 bp, synthetic construct
  misc_feature 1..21: note = oligonucleotide
  source 1..21: mol_type = other DNA
agcggccagccaggacccccc

SEQ ID NO: 1488 - DNA, 21 bp, synthetic construct
  misc_feature 1..21: note = oligonucleotide
  source 1..21: mol_type = other DNA
ggatcatgacagagaagcagg

SEQ ID NO: 1489 - DNA, 21 bp, synthetic construct
  misc_feature 1..21: note = oligonucleotide
  source 1..21: mol_type = other DNA
cgatgagattttttttggagg

SEQ ID NO: 1490 - DNA, 21 bp, synthetic construct
  misc_feature 1..21: note = oligonucleotide
  source 1..21: mol_type = other DNA
gtagtgtggtcggagctgccc

SEQ ID NO: 1491 - DNA, 21 bp, synthetic construct
  misc_feature 1..21: note = oligonucleotide
  source 1..21: mol_type = other DNA
cccctcctagccctattcagg

SEQ ID NO: 1492 - DNA, 21 bp, synthetic construct
  misc_feature 1..21: note = oligonucleotide
  source 1..21: mol_type = other DNA
gtgaggtcggagccagagggc

SEQ ID NO: 1493 - DNA, 21 bp, synthetic construct
  misc_feature 1..21: note = oligonucleotide
  source 1..21: mol_type = other DNA
gcgctatctctctttctggcc

SEQ ID NO: 1494 - DNA, 21 bp, synthetic construct
  misc_feature 1..21: note = oligonucleotide
  source 1..21: mol_type = other DNA
cctgcgctgcaactacagcat

SEQ ID NO: 1495 - DNA, 21 bp, synthetic construct
  misc_feature 1..21: note = oligonucleotide
  source 1..21: mol_type = other DNA
aaaaaaaataaaaataaacca

SEQ ID NO: 1496 - DNA, 21 bp, synthetic construct
  misc_feature 1..21: note = oligonucleotide
  source 1..21: mol_type = other DNA
catgggggggcgggtgttgag

SEQ ID NO: 1497 - DNA, 21 bp, synthetic construct
  misc_feature 1..21: note = oligonucleotide
  source 1..21: mol_type = other DNA
acccccccaccaaccccaggg

SEQ ID NO: 1498 - DNA, 21 bp, synthetic construct
  misc_feature 1..21: note = oligonucleotide
  source 1..21: mol_type = other DNA
catcccagagccagttgtcat

SEQ ID NO: 1499 - DNA, 21 bp, synthetic construct
  misc_feature 1..21: note = oligonucleotide
  source 1..21: mol_type = other DNA
attaaaaaaaaatcttcatta

SEQ ID NO: 1500 - DNA, 21 bp, synthetic construct
  misc_feature 1..21: note = oligonucleotide
  source 1..21: mol_type = other DNA
ggtggctacagtggagagggc

SEQ ID NO: 1501 - DNA, 21 bp, synthetic construct
  misc_feature 1..21: note = oligonucleotide
  source 1..21: mol_type = other DNA
ggtttctgagcgcataaagct

SEQ ID NO: 1502 - DNA, 21 bp, synthetic construct
  misc_feature 1..21: note = oligonucleotide
  source 1..21: mol_type = other DNA
ccattgccgtggtgcttggca

SEQ ID NO: 1503 - DNA, 21 bp, synthetic construct
  misc_feature 1..21: note = oligonucleotide
  source 1..21: mol_type = other DNA
ctgagatctcagatacgtgta

SEQ ID NO: 1504 - DNA, 21 bp, synthetic construct
  misc_feature 1..21: note = oligonucleotide
  source 1..21: mol_type = other DNA
gggggcccccatgactctggg

SEQ ID NO: 1505 - DNA, 21 bp, synthetic construct
  misc_feature 1..21: note = oligonucleotide
  source 1..21: mol_type = other DNA
ccgctccttcctcctggcttc

SEQ ID NO: 1506 - DNA, 21 bp, synthetic construct
  misc_feature 1..21: note = oligonucleotide
  source 1..21: mol_type = other DNA
cgcgctatctctctttctggc

SEQ ID NO: 1507 - DNA, 21 bp, synthetic construct
  misc_feature 1..21: note = oligonucleotide
  source 1..21: mol_type = other DNA
tagtgatcctcccccaccgaa

SEQ ID NO: 1508 - DNA, 21 bp, synthetic construct
  misc_feature 1..21: note = oligonucleotide
  source 1..21: mol_type = other DNA
ggtgcctgaggtgtgggggga

SEQ ID NO: 1509 - DNA, 21 bp, synthetic construct
  misc_feature 1..21: note = oligonucleotide
  source 1..21: mol_type = other DNA
gcggccagccaggaccccccc

SEQ ID NO: 1510 - DNA, 21 bp, synthetic construct
  misc_feature 1..21: note = oligonucleotide
  source 1..21: mol_type = other DNA
ggtggctacagtggagagggc

SEQ ID NO: 1511 - DNA, 21 bp, synthetic construct
  misc_feature 1..21: note = oligonucleotide
  source 1..21: mol_type = other DNA
gggatcatgacagagaagcag

SEQ ID NO: 1512 - DNA, 21 bp, synthetic construct
  misc_feature 1..21: note = oligonucleotide
  source 1..21: mol_type = other DNA
aataaactcaaaaaaaaaggt

SEQ ID NO: 1513 - DNA, 21 bp, synthetic construct
  misc_feature 1..21: note = oligonucleotide
  source 1..21: mol_type = other DNA
atgctaattcacaagtttttt

SEQ ID NO: 1514 - DNA, 21 bp, synthetic construct
  misc_feature 1..21: note = oligonucleotide
  source 1..21: mol_type = other DNA
taaactcaaaaaaaaaggtag

SEQ ID NO: 1515 - DNA, 21 bp, synthetic construct
  misc_feature 1..21: note = oligonucleotide
  source 1..21: mol_type = other DNA
aaaaaagaaatgctactcggt

SEQ ID NO: 1516 - DNA, 21 bp, synthetic construct
  misc_feature 1..21: note = oligonucleotide
  source 1..21: mol_type = other DNA
agcttatgtccagcagagctg

SEQ ID NO: 1517 - DNA, 21 bp, synthetic construct
  misc_feature 1..21: note = oligonucleotide
  source 1..21: mol_type = other DNA
gaataaactcaaaaaaaaagg

SEQ ID NO: 1518 - DNA, 21 bp, synthetic construct
  misc_feature 1..21: note = oligonucleotide
  source 1..21: mol_type = other DNA
cgcaggtcagggggtaggacc

SEQ ID NO: 1519 - DNA, 21 bp, synthetic construct
  misc_feature 1..21: note = oligonucleotide
  source 1..21: mol_type = other DNA
gcccgagttactgctgggggg

SEQ ID NO: 1520 - DNA, 21 bp, synthetic construct
  misc_feature 1..21: note = oligonucleotide
  source 1..21: mol_type = other DNA
cgagacgtgagtggaggccaa

SEQ ID NO: 1521 - DNA, 21 bp, synthetic construct
  misc_feature 1..21: note = oligonucleotide
  source 1..21: mol_type = other DNA
gatgagattttttttggaggg

SEQ ID NO: 1522 - DNA, 21 bp, synthetic construct
  misc_feature 1..21: note = oligonucleotide
  source 1..21: mol_type = other DNA
cgggggcggcactataataat

SEQ ID NO: 1523 - DNA, 21 bp, synthetic construct
  misc_feature 1..21: note = oligonucleotide
  source 1..21: mol_type = other DNA
aaaatgaaaaaaaaggggggt

SEQ ID NO: 1524 - DNA, 21 bp, synthetic construct
  misc_feature 1..21: note = oligonucleotide
  source 1..21: mol_type = other DNA
cccccaggctcctctgagact

SEQ ID NO: 1525 - DNA, 21 bp, synthetic construct
  misc_feature 1..21: note = oligonucleotide
  source 1..21: mol_type = other DNA
atgcaggcaggcaccgggtcg

SEQ ID NO: 1526 - DNA, 21 bp, synthetic construct
  misc_feature 1..21: note = oligonucleotide
  source 1..21: mol_type = other DNA
tccgatgagattttttttgga

SEQ ID NO: 1527 - DNA, 21 bp, synthetic construct
  misc_feature 1..21: note = oligonucleotide
  source 1..21: mol_type = other DNA
ggggtgcgcttgattctactt

SEQ ID NO: 1528 - DNA, 21 bp, synthetic construct
  misc_feature 1..21: note = oligonucleotide
  source 1..21: mol_type = other DNA
caaaagacccttaaaaaaaaa

SEQ ID NO: 1529 - DNA, 21 bp, synthetic construct
  misc_feature 1..21: note = oligonucleotide
  source 1..21: mol_type = other DNA
caaaagacccttaaaaaaaaa

SEQ ID NO: 1530 - DNA, 21 bp, synthetic construct
  misc_feature 1..21: note = oligonucleotide
  source 1..21: mol_type = other DNA
ttttgaggggcattttaaact

SEQ ID NO: 1531 - DNA, 21 bp, synthetic construct
  misc_feature 1..21: note = oligonucleotide
  source 1..21: mol_type = other DNA
ttggtgatacccccggactga

SEQ ID NO: 1532 - DNA, 21 bp, synthetic construct
  misc_feature 1..21: note = oligonucleotide
  source 1..21: mol_type = other DNA
gagggcttggggcgtactccg

SEQ ID NO: 1533 - DNA, 21 bp, synthetic construct
  misc_feature 1..21: note = oligonucleotide
  source 1..21: mol_type = other DNA
gggtgcgcttgattctacttc

SEQ ID NO: 1534 - DNA, 21 bp, synthetic construct
  misc_feature 1..21: note = oligonucleotide
  source 1..21: mol_type = other DNA
gtcattggggggatcatgaca

SEQ ID NO: 1535 - DNA, 21 bp, synthetic construct
  misc_feature 1..21: note = oligonucleotide
  source 1..21: mol_type = other DNA
aggaaaaaaaaggggtatatt

SEQ ID NO: 1536 - DNA, 21 bp, synthetic construct
  misc_feature 1..21: note = oligonucleotide
  source 1..21: mol_type = other DNA
agtgatcctcccccaccgaag

SEQ ID NO: 1537 - DNA, 21 bp, synthetic construct
  misc_feature 1..21: note = oligonucleotide
  source 1..21: mol_type = other DNA
aatagtgatcctcccccaccg

SEQ ID NO: 1538 - DNA, 21 bp, synthetic construct
  misc_feature 1..21: note = oligonucleotide
  source 1..21: mol_type = other DNA
cacctgggggtgtgcctgcag

SEQ ID NO: 1539 - DNA, 21 bp, synthetic construct
  misc_feature 1..21: note = oligonucleotide
  source 1..21: mol_type = other DNA
gctccttcctcctggcttccc

SEQ ID NO: 1540 - DNA, 21 bp, synthetic construct
  misc_feature 1..21: note = oligonucleotide
  source 1..21: mol_type = other DNA
aaaaaaaacaacatggctgca

SEQ ID NO: 1541 - DNA, 21 bp, synthetic construct
  misc_feature 1..21: note = oligonucleotide
  source 1..21: mol_type = other DNA
gccccttccaaagacccctgg

SEQ ID NO: 1542 - DNA, 21 bp, synthetic construct
  misc_feature 1..21: note = oligonucleotide
  source 1..21: mol_type = other DNA
ctgcagctccagggggcctgg

SEQ ID NO: 1543 - DNA, 21 bp, synthetic construct
  misc_feature 1..21: note = oligonucleotide
  source 1..21: mol_type = other DNA
ggggatcatgacagagaagca

SEQ ID NO: 1544 - DNA, 21 bp, synthetic construct
  misc_feature 1..21: note = oligonucleotide
  source 1..21: mol_type = other DNA
cccacatctggccgaagggct

SEQ ID NO: 1545 - DNA, 21 bp, synthetic construct
  misc_feature 1..21: note = oligonucleotide
  source 1..21: mol_type = other DNA
ccatgtgagcagcagccagcg

SEQ ID NO: 1546 - DNA, 21 bp, synthetic construct
  misc_feature 1..21: note = oligonucleotide
  source 1..21: mol_type = other DNA
ggatctcacgggagttcctcc

SEQ ID NO: 1547 - DNA, 21 bp, synthetic construct
  misc_feature 1..21: note = oligonucleotide
  source 1..21: mol_type = other DNA
gggtgcctgaggtgtgggggg

SEQ ID NO: 1548 - DNA, 21 bp, synthetic construct
  misc_feature 1..21: note = oligonucleotide
  source 1..21: mol_type = other DNA
gtccgatgagattttttttgg

SEQ ID NO: 1549 - DNA, 21 bp, synthetic construct
  misc_feature 1..21: note = oligonucleotide
  source 1..21: mol_type = other DNA
ggtacagggaactggactgtc

SEQ ID NO: 1550 - DNA, 21 bp, synthetic construct
  misc_feature 1..21: note = oligonucleotide
  source 1..21: mol_type = other DNA
caaaatgaaaaaaaagggggg

SEQ ID NO: 1551 - DNA, 21 bp, synthetic construct
  misc_feature 1..21: note = oligonucleotide
  source 1..21: mol_type = other DNA
cgctgcaactacagcatccgg

SEQ ID NO: 1552 - DNA, 21 bp, synthetic construct
  misc_feature 1..21: note = oligonucleotide
  source 1..21: mol_type = other DNA
gtgcctgaggtgtggggggat

SEQ ID NO: 1553 - DNA, 21 bp, synthetic construct
  misc_feature 1..21: note = oligonucleotide
  source 1..21: mol_type = other DNA
aggggaacctggaagttaaca

SEQ ID NO: 1554 - DNA, 21 bp, synthetic construct
  misc_feature 1..21: note = oligonucleotide
  source 1..21: mol_type = other DNA
ttccgctctgtcctgggtgac

SEQ ID NO: 1555 - DNA, 21 bp, synthetic construct
  misc_feature 1..21: note = oligonucleotide
  source 1..21: mol_type = other DNA
taagctattaccacaaaaaaa

SEQ ID NO: 1556 - DNA, 21 bp, synthetic construct
  misc_feature 1..21: note = oligonucleotide
  source 1..21: mol_type = other DNA
gggggatcatgacagagaagc

SEQ ID NO: 1557 - DNA, 21 bp, synthetic construct
  misc_feature 1..21: note = oligonucleotide
  source 1..21: mol_type = other DNA
ggctgcagctccagggggcct

SEQ ID NO: 1558 - DNA, 21 bp, synthetic construct
  misc_feature 1..21: note = oligonucleotide
  source 1..21: mol_type = other DNA
ttttctttttgagtcaggact

SEQ ID NO: 1559 - DNA, 21 bp, synthetic construct
  misc_feature 1..21: note = oligonucleotide
  source 1..21: mol_type = other DNA
tgggatttttaggtcagccca

SEQ ID NO: 1560 - DNA, 21 bp, synthetic construct
  misc_feature 1..21: note = oligonucleotide
  source 1..21: mol_type = other DNA
tcatttttttttaataagtcc

SEQ ID NO: 1561 - DNA, 21 bp, synthetic construct
  misc_feature 1..21: note = oligonucleotide
  source 1..21: mol_type = other DNA
aaaaaaagaaaaagaaaaaag

SEQ ID NO: 1562 - DNA, 21 bp, synthetic construct
  misc_feature 1..21: note = oligonucleotide
  source 1..21: mol_type = other DNA
gtgccccctgcttgtggtgca

SEQ ID NO: 1563 - DNA, 21 bp, synthetic construct
  misc_feature 1..21: note = oligonucleotide
  source 1..21: mol_type = other DNA
gtgtgcctgcgagccgggctg

SEQ ID NO: 1564 - DNA, 21 bp, synthetic construct
  misc_feature 1..21: note = oligonucleotide
  source 1..21: mol_type = other DNA
tggccaagggccagggagccc

SEQ ID NO: 1565 - DNA, 21 bp, synthetic construct
  misc_feature 1..21: note = oligonucleotide
  source 1..21: mol_type = other DNA
tggtgttttttgagatattct

SEQ ID NO: 1566 - DNA, 21 bp, synthetic construct
  misc_feature 1..21: note = oligonucleotide
  source 1..21: mol_type = other DNA
acgccttactctaactggcac

SEQ ID NO: 1567 - DNA, 21 bp, synthetic construct
  misc_feature 1..21: note = oligonucleotide
  source 1..21: mol_type = other DNA
ccgctccttcctcctggcttc

SEQ ID NO: 1568 - DNA, 21 bp, synthetic construct
  misc_feature 1..21: note = oligonucleotide
  source 1..21: mol_type = other DNA
tttttcttttgaaatctagtt

SEQ ID NO: 1569 - DNA, 21 bp, synthetic construct
  misc_feature 1..21: note = oligonucleotide
  source 1..21: mol_type = other DNA
aaaaaaacaaaaacctagatt

SEQ ID NO: 1570 - DNA, 21 bp, synthetic construct
  misc_feature 1..21: note = oligonucleotide
  source 1..21: mol_type = other DNA
ctggaaacactgcaggaaaca

SEQ ID NO: 1571 - DNA, 21 bp, synthetic construct
  misc_feature 1..21: note = oligonucleotide
  source 1..21: mol_type = other DNA
ggtgtcaggcaccggatctca

SEQ ID NO: 1572 - DNA, 21 bp, synthetic construct
  misc_feature 1..21: note = oligonucleotide
  source 1..21: mol_type = other DNA
tttggcactgtttattctttg

SEQ ID NO: 1573 - DNA, 21 bp, synthetic construct
  misc_feature 1..21: note = oligonucleotide
  source 1..21: mol_type = other DNA
gcgctatctctctttctggcc

SEQ ID NO: 1574 - DNA, 21 bp, synthetic construct
  misc_feature 1..21: note = oligonucleotide
  source 1..21: mol_type = other DNA
accatgtgagcagcagccagc

SEQ ID NO: 1575 - DNA, 21 bp, synthetic construct
  misc_feature 1..21: note = oligonucleotide
  source 1..21: mol_type = other DNA
gtctcaaaaaaaaaaaattta

SEQ ID NO: 1576 - DNA, 21 bp, synthetic construct
  misc_feature 1..21: note = oligonucleotide
  source 1..21: mol_type = other DNA
aaaaaaaaaacaaaacattta

SEQ ID NO: 1577 - DNA, 21 bp, synthetic construct
  misc_feature 1..21: note = oligonucleotide
  source 1..21: mol_type = other DNA
catgtgagcagcagccagcgg

SEQ ID NO: 1578 - DNA, 21 bp, synthetic construct
  misc_feature 1..21: note = oligonucleotide
  source 1..21: mol_type = other DNA
cgcgctatctctctttctggc

SEQ ID NO: 1579 - DNA, 21 bp, synthetic construct
  misc_feature 1..21: note = oligonucleotide
  source 1..21: mol_type = other DNA
ctgcagctccagggggcctgg

SEQ ID NO: 1580 - DNA, 21 bp, synthetic construct
  misc_feature 1..21: note = oligonucleotide
  source 1..21: mol_type = other DNA
caccatgtgagcagcagccag

SEQ ID NO: 1581 - DNA, 21 bp, synthetic construct
  misc_feature 1..21: note = oligonucleotide
  source 1..21: mol_type = other DNA
tttagtccccccctacagctc

SEQ ID NO: 1582 - DNA, 21 bp, synthetic construct
  misc_feature 1..21: note = oligonucleotide
  source 1..21: mol_type = other DNA
ggagggctggggggtatcacc

SEQ ID NO: 1583 - DNA, 21 bp, synthetic construct
  misc_feature 1..21: note = oligonucleotide
  source 1..21: mol_type = other DNA
caagcctccaggcctctcggc

SEQ ID NO: 1584 - DNA, 21 bp, synthetic construct
  misc_feature 1..21: note = oligonucleotide
  source 1..21: mol_type = other DNA
gtttcctgcagtgtttccagg

SEQ ID NO: 1585 - DNA, 21 bp, synthetic construct
  misc_feature 1..21: note = oligonucleotide
  source 1..21: mol_type = other DNA
ttaaaatgagaccaaaccctc

SEQ ID NO: 1586 - DNA, 21 bp, synthetic construct
  misc_feature 1..21: note = oligonucleotide
  source 1..21: mol_type = other DNA
gtggcccgggtggaggtggcg

SEQ ID NO: 1587 - DNA, 21 bp, synthetic construct
  misc_feature 1..21: note = oligonucleotide
  source 1..21: mol_type = other DNA
gggctggcgggggcacaggag

SEQ ID NO: 1588 - DNA, 21 bp, synthetic construct
  misc_feature 1..21: note = oligonucleotide
  source 1..21: mol_type = other DNA
gtgccactggggggctgctcc

SEQ ID NO: 1589 - DNA, 21 bp, synthetic construct
  misc_feature 1..21: note = oligonucleotide
  source 1..21: mol_type = other DNA
agggctggggggtatcaccca

SEQ ID NO: 1590 - DNA, 21 bp, synthetic construct
  misc_feature 1..21: note = oligonucleotide
  source 1..21: mol_type = other DNA
gtgtcaggcaccggatctcac

SEQ ID NO: 1591 - DNA, 21 bp, synthetic construct
  misc_feature 1..21: note = oligonucleotide
  source 1..21: mol_type = other DNA
aggggaacctggaagttaaca

SEQ ID NO: 1592 - DNA, 21 bp, synthetic construct
  misc_feature 1..21: note = oligonucleotide
  source 1..21: mol_type = other DNA
cctggaaacactgcaggaaac

SEQ ID NO: 1593 - DNA, 21 bp, synthetic construct
  misc_feature 1..21: note = oligonucleotide
  source 1..21: mol_type = other DNA
gggttggtgggggggtgcaga

SEQ ID NO: 1594 - DNA, 21 bp, synthetic construct
  misc_feature 1..21: note = oligonucleotide
  source 1..21: mol_type = other DNA
gagggcttggggcgtactccg

SEQ ID NO: 1595 - DNA, 21 bp, synthetic construct
  misc_feature 1..21: note = oligonucleotide
  source 1..21: mol_type = other DNA
atgatttttttacacagtctc

SEQ ID NO: 1596 - DNA, 21 bp, synthetic construct
  misc_feature 1..21: note = oligonucleotide
  source 1..21: mol_type = other DNA
acacatccccactgtgccaga

SEQ ID NO: 1597 - DNA, 21 bp, synthetic construct
  misc_feature 1..21: note = oligonucleotide
  source 1..21: mol_type = other DNA
ggctgctggtggtggtggtgg

SEQ ID NO: 1598 - DNA, 21 bp, synthetic construct
  misc_feature 1..21: note = oligonucleotide
  source 1..21: mol_type = other DNA
ggggggtagctgccatacagg

SEQ ID NO: 1599 - DNA, 21 bp, synthetic construct
  misc_feature 1..21: note = oligonucleotide
  source 1..21: mol_type = other DNA
cccagtgggtgccgggaccgg

SEQ ID NO: 1600 - DNA, 21 bp, synthetic construct
  misc_feature 1..21: note = oligonucleotide
  source 1..21: mol_type = other DNA
atgcaggcaggcaccgggtcg

SEQ ID NO: 1601 - DNA, 21 bp, synthetic construct
  misc_feature 1..21: note = oligonucleotide
  source 1..21: mol_type = other DNA
cccattaaaaaaaaaaagtct

SEQ ID NO: 1602 - DNA, 21 bp, synthetic construct
  misc_feature 1..21: note = oligonucleotide
  source 1..21: mol_type = other DNA
gaaaaaaaaaacaaaacattt

SEQ ID NO: 1603 - DNA, 21 bp, synthetic construct
  misc_feature 1..21: note = oligonucleotide
  source 1..21: mol_type = other DNA
tggttagcatttttttttttt

SEQ ID NO: 1604 - DNA, 21 bp, synthetic construct
  misc_feature 1..21: note = oligonucleotide
  source 1..21: mol_type = other DNA
acaaaaatcacattctaatgg

SEQ ID NO: 1605 - DNA, 21 bp, synthetic construct
  misc_feature 1..21: note = oligonucleotide
  source 1..21: mol_type = other DNA
acaaagtttaaacatgggggg

SEQ ID NO: 1606 - DNA, 21 bp, synthetic construct
  misc_feature 1..21: note = oligonucleotide
  source 1..21: mol_type = other DNA
tttttcccagtgttaggtgaa

SEQ ID NO: 1607 - DNA, 21 bp, synthetic construct
  misc_feature 1..21: note = oligonucleotide
  source 1..21: mol_type = other DNA
agcgcgagcacagctaaggcc

SEQ ID NO: 1608 - DNA, 21 bp, synthetic construct
  misc_feature 1..21: note = oligonucleotide
  source 1..21: mol_type = other DNA
tcggagccaggcatgtcacca

SEQ ID NO: 1609 - DNA, 21 bp, synthetic construct
  misc_feature 1..21: note = oligonucleotide
  source 1..21: mol_type = other DNA
ttcctctgtgggtgtcgggca

SEQ ID NO: 1610 - DNA, 21 bp, synthetic construct
  misc_feature 1..21: note = oligonucleotide
  source 1..21: mol_type = other DNA
gggggttgcactggagcgtca

SEQ ID NO: 1611 - DNA, 21 bp, synthetic construct
  misc_feature 1..21: note = oligonucleotide
  source 1..21: mol_type = other DNA
gtctcaaaaaaaaaaaattta

SEQ ID NO: 1612 - DNA, 21 bp, synthetic construct
  misc_feature 1..21: note = oligonucleotide
  source 1..21: mol_type = other DNA
caagaaaaagaaagacaaaaa

SEQ ID NO: 1613 - DNA, 21 bp, synthetic construct
  misc_feature 1..21: note = oligonucleotide
  source 1..21: mol_type = other DNA
aaaaagaaagacaaaaaaaga

SEQ ID NO: 1614 - DNA, 21 bp, synthetic construct
  misc_feature 1..21: note = oligonucleotide
  source 1..21: mol_type = other DNA
gtcaaacacttcatctggtgg

SEQ ID NO: 1615 - DNA, 21 bp, synthetic construct
  misc_feature 1..21: note = oligonucleotide
  source 1..21: mol_type = other DNA
gagctgtgagtcctgacgggg

SEQ ID NO: 1616 - DNA, 21 bp, synthetic construct
  misc_feature 1..21: note = oligonucleotide
  source 1..21: mol_type = other DNA
gtgccactggggggctgctcc

SEQ ID NO: 1617 - DNA, 21 bp, synthetic construct
  misc_feature 1..21: note = oligonucleotide
  source 1..21: mol_type = other DNA
gaagagctgtgagtcctgacg

SEQ ID NO: 1618 - DNA, 21 bp, synthetic construct
  misc_feature 1..21: note = oligonucleotide
  source 1..21: mol_type = other DNA
ccgcgttcaccctgcattttt

SEQ ID NO: 1619 - DNA, 21 bp, synthetic construct
  misc_feature 1..21: note = oligonucleotide
  source 1..21: mol_type = other DNA
ccctggaaacactgcaggaaa

SEQ ID NO: 1620 - DNA, 21 bp, synthetic construct
  misc_feature 1..21: note = oligonucleotide
  source 1..21: mol_type = other DNA
ctggaaacactgcaggaaaca

SEQ ID NO: 1621 - DNA, 21 bp, synthetic construct
  misc_feature 1..21: note = oligonucleotide
  source 1..21: mol_type = other DNA
cctggaaacactgcaggaaac

SEQ ID NO: 1622 - DNA, 21 bp, synthetic construct
  misc_feature 1..21: note = oligonucleotide
  source 1..21: mol_type = other DNA
gtgagcagcagccagcggggg

SEQ ID NO: 1623 - DNA, 21 bp, synthetic construct
  misc_feature 1..21: note = oligonucleotide
  source 1..21: mol_type = other DNA
ttgtttttttgtttttttttg

SEQ ID NO: 1624 - DNA, 21 bp, synthetic construct
  misc_feature 1..21: note = oligonucleotide
  source 1..21: mol_type = other DNA
ctggctgtggggagggcggtg

SEQ ID NO: 1625 - DNA, 21 bp, synthetic construct
  misc_feature 1..21: note = oligonucleotide
  source 1..21: mol_type = other DNA
ttttttcccccagtgagggga

SEQ ID NO: 1626 - DNA, 21 bp, synthetic construct
  misc_feature 1..21: note = oligonucleotide
  source 1..21: mol_type = other DNA
tttttcccccagtgaggggac

SEQ ID NO: 1627 - DNA, 21 bp, synthetic construct
  misc_feature 1..21: note = oligonucleotide
  source 1..21: mol_type = other DNA
cccccaagggcctccgggatg

SEQ ID NO: 1628 - DNA, 21 bp, synthetic construct
  misc_feature 1..21: note = oligonucleotide
  source 1..21: mol_type = other DNA
tcatgtgtgtctgcatatgtg

SEQ ID NO: 1629 - DNA, 21 bp, synthetic construct
  misc_feature 1..21: note = oligonucleotide
  source 1..21: mol_type = other DNA
ttaatttaaaaaaaaaaagaa

SEQ ID NO: 1630 - DNA, 21 bp, synthetic construct
  misc_feature 1..21: note = oligonucleotide
  source 1..21: mol_type = other DNA
gaggggacatgcaggcaggca

SEQ ID NO: 1631 - DNA, 21 bp, synthetic construct
  misc_feature 1..21: note = oligonucleotide
  source 1..21: mol_type = other DNA
tttcccccagtgaggggactg

SEQ ID NO: 1632 - DNA, 21 bp, synthetic construct
  misc_feature 1..21: note = oligonucleotide
  source 1..21: mol_type = other DNA
tgtttttttgtttttttttga

SEQ ID NO: 1633 - DNA, 21 bp, synthetic construct
  misc_feature 1..21: note = oligonucleotide
  source 1..21: mol_type = other DNA
gggagcccaggggtggctaca

SEQ ID NO: 1634 - DNA, 21 bp, synthetic construct
  misc_feature 1..21: note = oligonucleotide
  source 1..21: mol_type = other DNA
cccaaggcgcggtgccccaga

SEQ ID NO: 1635 - DNA, 21 bp, synthetic construct
  misc_feature 1..21: note = oligonucleotide
  source 1..21: mol_type = other DNA
agcagagccgagtgtggaagt

SEQ ID NO: 1636 - DNA, 21 bp, synthetic construct
  misc_feature 1..21: note = oligonucleotide
  source 1..21: mol_type = other DNA
gcgctatctctctttctggcc

SEQ ID NO: 1637 - DNA, 21 bp, synthetic construct
  misc_feature 1..21: note = oligonucleotide
  source 1..21: mol_type = other DNA
ttcctctgtgggtgtcgggca

SEQ ID NO: 1638 - DNA, 21 bp, synthetic construct
  misc_feature 1..21: note = oligonucleotide
  source 1..21: mol_type = other DNA
ggtggtggggggtgtcttctg

SEQ ID NO: 1639 - DNA, 21 bp, synthetic construct
  misc_feature 1..21: note = oligonucleotide
  source 1..21: mol_type = other DNA
cgttgatggggggtagatctg

SEQ ID NO: 1640 - DNA, 21 bp, synthetic construct
  misc_feature 1..21: note = oligonucleotide
  source 1..21: mol_type = other DNA
tcctcataaataaaaaaaaac

SEQ ID NO: 1641 - DNA, 21 bp, synthetic construct
  misc_feature 1..21: note = oligonucleotide
  source 1..21: mol_type = other DNA
ccaaggcgcggtgccccagag

SEQ ID NO: 1642 - DNA, 21 bp, synthetic construct
  misc_feature 1..21: note = oligonucleotide
  source 1..21: mol_type = other DNA
cttcgacgaaaccgaccactt

SEQ ID NO: 1643 - DNA, 21 bp, synthetic construct
  misc_feature 1..21: note = oligonucleotide
  source 1..21: mol_type = other DNA
agttgaaaaaaaaagagtgac

SEQ ID NO: 1644 - DNA, 21 bp, synthetic construct
  misc_feature 1..21: note = oligonucleotide
  source 1..21: mol_type = other DNA
tagaaaaatgagtagaaagca

SEQ ID NO: 1645 - DNA, 21 bp, synthetic construct
  misc_feature 1..21: note = oligonucleotide
  source 1..21: mol_type = other DNA
cctcatgcccaccccctggct

SEQ ID NO: 1646 - DNA, 21 bp, synthetic construct
  misc_feature 1..21: note = oligonucleotide
  source 1..21: mol_type = other DNA
aaaaaaacaaaaacctagatt

SEQ ID NO: 1647 - DNA, 21 bp, synthetic construct
  misc_feature 1..21: note = oligonucleotide
  source 1..21: mol_type = other DNA
gtacccccgcctgaagacctt

SEQ ID NO: 1648 - DNA, 21 bp, synthetic construct
  misc_feature 1..21: note = oligonucleotide
  source 1..21: mol_type = other DNA
tgatggggggtagatctgagg

SEQ ID NO: 1649 - DNA, 21 bp, synthetic construct
  misc_feature 1..21: note = oligonucleotide
  source 1..21: mol_type = other DNA
gactgtttgaaaaaaaaaaat

SEQ ID NO: 1650 - DNA, 21 bp, synthetic construct
  misc_feature 1..21: note = oligonucleotide
  source 1..21: mol_type = other DNA
cccattaaaaaaaaaaagtct

SEQ ID NO: 1651 - DNA, 21 bp, synthetic construct
  misc_feature 1..21: note = oligonucleotide
  source 1..21: mol_type = other DNA
ggagacacccccaagggcctc

SEQ ID NO: 1652 - DNA, 21 bp, synthetic construct
  misc_feature 1..21: note = oligonucleotide
  source 1..21: mol_type = other DNA
ttttttgacaattaaattggg

SEQ ID NO: 1653 - DNA, 21 bp, synthetic construct
  misc_feature 1..21: note = oligonucleotide
  source 1..21: mol_type = other DNA
ccgccgcagcagagccgagtg

SEQ ID NO: 1654 - DNA, 21 bp, synthetic construct
  misc_feature 1..21: note = oligonucleotide
  source 1..21: mol_type = other DNA
tgcacccccccttgaagagct

SEQ ID NO: 1655 - DNA, 21 bp, synthetic construct
  misc_feature 1..21: note = oligonucleotide
  source 1..21: mol_type = other DNA
ttttgagtcaggactatggag

SEQ ID NO: 1656 - DNA, 21 bp, synthetic construct
  misc_feature 1..21: note = oligonucleotide
  source 1..21: mol_type = other DNA
aaatcttttaaaagctgccat

SEQ ID NO: 1657 - DNA, 21 bp, synthetic construct
  misc_feature 1..21: note = oligonucleotide
  source 1..21: mol_type = other DNA
cacgaactccttccgctcccc

SEQ ID NO: 1658 - DNA, 21 bp, synthetic construct
  misc_feature 1..21: note = oligonucleotide
  source 1..21: mol_type = other DNA
atttttttttgctaaatactt

SEQ ID NO: 1659 - DNA, 21 bp, synthetic construct
  misc_feature 1..21: note = oligonucleotide
  source 1..21: mol_type = other DNA
tcctgaatacaccgacacctg

SEQ ID NO: 1660 - DNA, 21 bp, synthetic construct
  misc_feature 1..21: note = oligonucleotide
  source 1..21: mol_type = other DNA
tttttttttctcggtggctct

SEQ ID NO: 1661 - DNA, 21 bp, synthetic construct
  misc_feature 1..21: note = oligonucleotide
  source 1..21: mol_type = other DNA
acccccggactgagcgaggaa

SEQ ID NO: 1662 - DNA, 21 bp, synthetic construct
  misc_feature 1..21: note = oligonucleotide
  source 1..21: mol_type = other DNA
atacccccggactgagcgagg

SEQ ID NO: 1663 - DNA, 21 bp, synthetic construct
  misc_feature 1..21: note = oligonucleotide
  source 1..21: mol_type = other DNA
tggggttcggcgaccgtcagg

SEQ ID NO: 1664 - DNA, 21 bp, synthetic construct
  misc_feature 1..21: note = oligonucleotide
  source 1..21: mol_type = other DNA
ctaaaaggagggcaagaggca

SEQ ID NO: 1665 - DNA, 21 bp, synthetic construct
  misc_feature 1..21: note = oligonucleotide
  source 1..21: mol_type = other DNA
tttttttttctcggtggctct

SEQ ID NO: 1666 - DNA, 21 bp, synthetic construct
  misc_feature 1..21: note = oligonucleotide
  source 1..21: mol_type = other DNA
attgctgagtccattctggaa

SEQ ID NO: 1667 - DNA, 21 bp, synthetic construct
  misc_feature 1..21: note = oligonucleotide
  source 1..21: mol_type = other DNA
ggtacccccgcctgaagacct

SEQ ID NO: 1668 - DNA, 21 bp, synthetic construct
  misc_feature 1..21: note = oligonucleotide
  source 1..21: mol_type = other DNA
aataatactttttttttttag

SEQ ID NO: 1669 - DNA, 21 bp, synthetic construct
  misc_feature 1..21: note = oligonucleotide
  source 1..21: mol_type = other DNA
aagcctgggcaggggtggctc

SEQ ID NO: 1670 - DNA, 21 bp, synthetic construct
  misc_feature 1..21: note = oligonucleotide
  source 1..21: mol_type = other DNA
ggggaaaaaacggaaataact

SEQ ID NO: 1671 - DNA, 21 bp, synthetic construct
  misc_feature 1..21: note = oligonucleotide
  source 1..21: mol_type = other DNA
ccccagtgggtgccgggaccg

SEQ ID NO: 1672 - DNA, 21 bp, synthetic construct
  misc_feature 1..21: note = oligonucleotide
  source 1..21: mol_type = other DNA
ggtgagcagaggggggtgtag

SEQ ID NO: 1673 - DNA, 21 bp, synthetic construct
  misc_feature 1..21: note = oligonucleotide
  source 1..21: mol_type = other DNA
ggatattgaaagtgtgaaaaa

SEQ ID NO: 1674 - DNA, 21 bp, synthetic construct
  misc_feature 1..21: note = oligonucleotide
  source 1..21: mol_type = other DNA
ccctggaaacactgcaggaaa

SEQ ID NO: 1675 - DNA, 21 bp, synthetic construct
  misc_feature 1..21: note = oligonucleotide
  source 1..21: mol_type = other DNA
tgagcagaggggggtgtagtc

SEQ ID NO: 1676 - DNA, 21 bp, synthetic construct
  misc_feature 1..21: note = oligonucleotide
  source 1..21: mol_type = other DNA
ggaagaggggtggcgtgacct

SEQ ID NO: 1677 - DNA, 21 bp, synthetic construct
  misc_feature 1..21: note = oligonucleotide
  source 1..21: mol_type = other DNA
ggaagaggggtggtgtcacct

SEQ ID NO: 1678 - DNA, 21 bp, synthetic construct
  misc_feature 1..21: note = oligonucleotide
  source 1..21: mol_type = other DNA
ggaagaggggtggtgtcacct

SEQ ID NO: 1679 - DNA, 21 bp, synthetic construct
  misc_feature 1..21: note = oligonucleotide
  source 1..21: mol_type = other DNA
ggaagagtcgtggtgtcaccg

SEQ ID NO: 1680 - DNA, 21 bp, synthetic construct
  misc_feature 1..21: note = oligonucleotide
  source 1..21: mol_type = other DNA
cttctccttccccacacactg

SEQ ID NO: 1681 - DNA, 21 bp, synthetic construct
  misc_feature 1..21: note = oligonucleotide
  source 1..21: mol_type = other DNA
ttctccttccccacacactgc

SEQ ID NO: 1682 - DNA, 21 bp, synthetic construct
  misc_feature 1..21: note = oligonucleotide
  source 1..21: mol_type = other DNA
tgaggaagcgccggtgacagg

SEQ ID NO: 1683 - DNA, 21 bp, synthetic construct
  misc_feature 1..21: note = oligonucleotide
  source 1..21: mol_type = other DNA
cgtaggcttcctggagggagg

SEQ ID NO: 1684 - DNA, 21 bp, synthetic construct
  misc_feature 1..21: note = oligonucleotide
  source 1..21: mol_type = other DNA
tcacgtaggcttcctggaggg

SEQ ID NO: 1685 - DNA, 20 bp, synthetic construct
  misc_feature 1..20: note = oligonucleotide
  source 1..20: mol_type = other DNA
gaagagtcgtggtgtcaccg

SEQ ID NO: 1686 - DNA, 21 bp, synthetic construct
  misc_feature 1..21: note = oligonucleotide
  source 1..21: mol_type = other DNA
gattttgggcgggccaaactg

SEQ ID NO: 1687 - DNA, 47 bp, Homo sapiens
  source 1..47: mol_type = unassigned DNA
ctgaggaagtgtcggtgacaggaagaggggtggcgtgaccggtggat

SEQ ID NO: 1688 - DNA, 45 bp, Homo sapiens
  source 1..45: mol_type = unassigned DNA
cgtcggtgacaggaagaggggtggtgtcacctgtggatactgagg

SEQ ID NO: 1689 - protein, 6 aa, synthetic construct
  REGION 1..6: note = peptide
GGSGGS

SEQ ID NO: 1690 - protein, 17 aa, synthetic construct
  REGION 1..17: note = peptide
AAEPKSSDKTHTCPPCP